SEQUENCE LISTING (51 sequences)

SEQ ID NO: 1 - DNA, 20 bp, Homo Sapiens
  gene (1)..(20): ABL2
aaatcagtgatgtggtgcag

SEQ ID NO: 2 - DNA, 20 bp, Homo Sapiens
  gene (1)..(20): ABL2
tgtacaggaaatcagtgatg

SEQ ID NO: 3 - DNA, 20 bp, Homo Sapiens
  gene (1)..(20): BCL9
atgtccatgcagggcatgat

SEQ ID NO: 4 - DNA, 20 bp, Homo Sapiens
  gene (1)..(20): BCL9
gggatcatgatgttctgttg

SEQ ID NO: 5 - DNA, 20 bp, Homo Sapiens
  gene (1)..(20): BIRC5
catcgagcagctggctgcca

SEQ ID NO: 6 - DNA, 20 bp, Homo Sapiens
  gene (1)..(20): BIRC5
cggccagaggcctcaatcca

SEQ ID NO: 7 - DNA, 20 bp, Homo Sapiens
  gene (1)..(20): BIRC5
cagctggctgccatggattg

SEQ ID NO: 8 - DNA, 20 bp, Homo Sapiens
  gene (1)..(20): CCNE1
agcggtaagaagcagagcag

SEQ ID NO: 9 - DNA, 20 bp, Homo Sapiens
  gene (1)..(20): CCNE1
taagaagcagagcagcgggc

SEQ ID NO: 10 - DNA, 20 bp, Homo Sapiens
  gene (1)..(20): CEP55
tcgcgatctgcttgtccatg

SEQ ID NO: 11 - DNA, 20 bp, Homo Sapiens
  gene (1)..(20): CEP55
actttgaacagtattccaca

SEQ ID NO: 12 - DNA, 20 bp, Homo Sapiens
  gene (1)..(20): CSF1R
aacaactatcagttctgctg

SEQ ID NO: 13 - DNA, 20 bp, Homo Sapiens
  gene (1)..(20): FUS
tgagcacagacaggatcgca

SEQ ID NO: 14 - DNA, 20 bp, Homo Sapiens
  gene (1)..(20): FUS
acagacaggatcgcagggag

SEQ ID NO: 15 - DNA, 20 bp, Homo Sapiens
  gene (1)..(20): IRF2
aggcttaacagctcttgacg

SEQ ID NO: 16 - DNA, 20 bp, Homo Sapiens
  gene (1)..(20): IRF2
ggcttaacagctcttgacgc

SEQ ID NO: 17 - DNA, 20 bp, Homo Sapiens
  gene (1)..(20): IRF2
gaaaacatcggatatcaccc

SEQ ID NO: 18 - DNA, 20 bp, Homo Sapiens
  gene (1)..(20): MYB
acgtgaatgcattctcagcc

SEQ ID NO: 19 - DNA, 20 bp, Homo Sapiens
  gene (1)..(20): MYB
tgcattctcagcccggacgc

SEQ ID NO: 20 - DNA, 20 bp, Homo Sapiens
  gene (1)..(20): MYB
tgtctcacatgaccagcgtc

SEQ ID NO: 21 - DNA, 20 bp, Homo Sapiens
  gene (1)..(20): NUMA1
ctttcaggcaaagcactaaa

SEQ ID NO: 22 - DNA, 20 bp, Homo Sapiens
  gene (1)..(20): NUMA1
tctttcaggcaaagcactaa

SEQ ID NO: 23 - DNA, 20 bp, Homo Sapiens
  gene (1)..(20): NUMA1
ctgaaagagacacagtcaca

SEQ ID NO: 24 - DNA, 20 bp, Homo Sapiens
  gene (1)..(20): SALL4
acaagattgcggtcagctaa

SEQ ID NO: 25 - DNA, 20 bp, Homo Sapiens
  gene (1)..(20): SEMA3E
cacacgctggactcctgatg

SEQ ID NO: 26 - DNA, 20 bp, Homo Sapiens
  gene (1)..(20): SEMA3E
ccgcctgcccaggcacacgc

SEQ ID NO: 27 - DNA, 20 bp, Homo Sapiens
  gene (1)..(20): SEMA3E
caggagtccagcgtgtgcct

SEQ ID NO: 28 - DNA, 20 bp, Homo Sapiens
  gene (1)..(20): SEMA3E
ctgagcattaccgcctgccc

SEQ ID NO: 29 - DNA, 20 bp, Homo Sapiens
  gene (1)..(20): SERTAD2
cacatcatggaggtgcttgt

SEQ ID NO: 30 - DNA, 20 bp, Homo Sapiens
  gene (1)..(20): SERTAD2
agagctggaccacatcatgg

SEQ ID NO: 31 - DNA, 20 bp, Homo Sapiens
  gene (1)..(20): SPHK1
ggtcataagggctcttctgg

SEQ ID NO: 32 - DNA, 20 bp, Homo Sapiens
  gene (1)..(20): SPHK1
gcgcggcccaggggtcataa

SEQ ID NO: 33 - DNA, 20 bp, Homo Sapiens
  gene (1)..(20): TACC3
tggacagcttgtgggaagcg

SEQ ID NO: 34 - DNA, 20 bp, Homo Sapiens
  gene (1)..(20): TACC3
tcactgcctggacagcttgt

SEQ ID NO: 35 - DNA, 20 bp, Homo Sapiens
  gene (1)..(20): TACC3
tcgcttcccacaagctgtcc

SEQ ID NO: 36 - DNA, 20 bp, Homo Sapiens
  gene (1)..(20): TIAM1
ctcagtgttcagtttcctgg

SEQ ID NO: 37 - DNA, 20 bp, Homo Sapiens
  gene (1)..(20): TIAM1
tcagtgttcagtttcctgga

SEQ ID NO: 38 - DNA, 20 bp, Homo Sapiens
  gene (1)..(20): TIAM1
cagtgttcagtttcctggag

SEQ ID NO: 39 - DNA, 20 bp, Homo Sapiens
  gene (1)..(20): CTTN
gcagctccacatagttggct

SEQ ID NO: 40 - DNA, 20 bp, Homo Sapiens
  gene (1)..(20): CTTN
gctcttcccagccaactatg

SEQ ID NO: 41 - DNA, 20 bp, Homo Sapiens
  gene (1)..(20): CTTN
ctatgtggagctgcggcagt

SEQ ID NO: 42 - DNA, 20 bp, Homo Sapiens
  gene (1)..(20): CTTN
tatgtggagctgcggcagta

SEQ ID NO: 43 - protein, 25 aa, Homo sapiens
  MISC_FEATURE: NEFH-WT
KSSSTDQKDSKPPEKATEDKAAKGK

SEQ ID NO: 44 - protein, 63 aa, Homo sapiens
  MISC_FEATURE: ASP1004Glnfs*56
KSSSTDQQASREGHRRQGRQGEVRQGERNIRNSQRNSEESRSSRIRVTQFSLFLSLCKKKLLR

SEQ ID NO: 45 - protein, 66 aa, Homo sapiens
  MISC_FEATURE: Pro1008Alafs*54
KSSSTDQKDSKPASREGHRRQGRQGEVRQGERNIRNSQRNSEESRSSRIRVTQFSLFLSLCKKKLR

SEQ ID NO: 46 - protein, 23 aa, Homo sapiens
  MISC_FEATURE: NEFH-3′UTR ORF1
GREKGTSGTAKETQKSPGAQGSE

SEQ ID NO: 47 - protein, 33 aa, Homo sapiens
  MISC_FEATURE: NEFH-3′UTR ORF2
AGRKEHPEQPKKLRRVPELKDQSNTIFTFSVFM

SEQ ID NO: 48 - protein, 40 aa, Homo sapiens
  MISC_FEATURE: NEFH-3′UTR ORF3
RQGERNIRNSQRNSEESRSSRIRVTQFSLFLSLCKKKLLR

SEQ ID NO: 49 - protein, 33 aa, Homo sapiens
  MISC_FEATURE: NEFL-3′UTR ORF1
TPISLIISGIILPKSGQPHHQPTNQLSSRFYVN

SEQ ID NO: 50 - protein, 36 aa, Homo sapiens
  MISC_FEATURE: NEFL-3′UTR ORF2
PHFLNYFRNNSPEIRSTPSPTNQPVEFQILCELSGR

SEQ ID NO: 51 - protein, 4 aa, Homo sapiens
  MISC_FEATURE: NEFL-3′UTR ORF3
PPFP

(19) United States
(12) Patent Application Publication
(10) Pub. No.: US 2021/0154267 A1
(43) Pub. Date: May 27, 2021

(54) TREATMENT OF CANCER BY ACTIVATING ENDOGENOUS CRYPTIC AMYLOIDOGENIC AGGREGATING PEPTIDES

(71) Applicant: UNIVERSITY OF MIAMI, Miami, FL (US)

(72) Inventors: Stephan L. Zuchner, Pinecrest, FL (US); Adriana Rebelo, Miami, FL (US)

(21) Appl. No.: 16/087,405

(22) PCT Filed: Mar. 23, 2017
(86) PCT No.: PCT/US2017/023901

§ 371 Date: Sep. 21, 2018

Related U.S. Application Data
(60) Provisional application No. 62/312,419, filed on Mar. 23, 2016.

(51) Int. Cl. A61K 38/17; A61P 35/00; A61K 48/00; A61K 38/46; A61K 31/7105; C12Q 1/6886; A61K 9/00; C12N 15/86

(52) U.S. Cl. CPC A61K 38/1709; A61P 35/00; A61K 48/0075; A61K 48/0066; A61K 38/465; C12Q 2600/158; C12Q 1/6886; A61K 9/0085; A61K 9/0048; C12N 15/86; C12Q 2600/106; A61K 31/7105

(57) ABSTRACT

The present disclosure relates, in general, to therapy using toxic amyloidogenic aggregating peptides for treating cancer or preventing recurrence of cancer diseases such as lung cancer, prostate cancer, breast cancer, hepatocellular cancer, esophageal cancer, colorectal cancer, pancreatic cancer, bladder cancer, kidney cancer, ovarian cancer, stomach cancer, fibrotic cancer, glioma and melanoma, and metastases thereof.

### CROSS-REFERENCE TO RELATED APPLICATIONS

This application is a U.S. national phase of International Application No. PCT/US2017/023901 filed Mar. 23, 2017, which claims priority to U.S. Provisional Patent Application No. 62/312,419 filed Mar. 23, 2016.

### INCORPORATION BY REFERENCE OF MATERIALS SUBMITTED ELECTRONICALLY

This application contains, as a separate part of the disclosure, a Sequence Listing in computer readable form (Filename: 50471A_Seqlisting.txt; Size: 12,797 bytes; Created: Sep. 21, 2018), which is incorporated by reference in its entirety.

### FIELD OF THE INVENTION

The invention relates, in general, to materials and methods for cancer treatment and prevention.

### BACKGROUND

Cancer is a group of diseases characterized by the uncontrolled growth and spread of abnormal cells. Cancer is the second leading cause of human death next to coronary disease (Health, United States, 2014: At a Glance, National Center for Health Statistics, CDC). According to statistics from the American Cancer Society, approximately 600,000 Americans are expected to die of cancer in 2016 (Cancer Facts & Figures 2016, American Cancer Society). Cancer therapeutic resistance occurs as cancers develop resistance to treatments such as chemotherapy, radiotherapy and targeted therapies, through numerous different mechanisms. Therefore, there is an urgent need for the development of effective anticancer agents to target difficult to treat cancers that fail to respond to current cancer therapy.

Abnormal protein aggregation is observed as a newly uncovered general response to cellular stress (Audas et al., Developmental Cell (2016)) and a long recognized mechanism in neurodegenerative disorders (Koo et al., PNAS (1999) 96: 9989-90). Protein aggregates usually adopt high-ordered beta sheet quaternary structures forming insoluble fibrils termed amyloids. The term “amyloid” is generically used for all proteins capable of forming large, insoluble fibrils. Amyloid pathology has been extensively described in diseases correlated with aging and aging itself. In cancer, a disease of aging, prion-like aggregation of mutant p53 has been observed (Costa et al., Cold Spring Harb Perspect Biol. (2016) 8(10): a023614). Parkinson disease is characterized by inclusions known as Lewy bodies present in the cytoplasm of neurons. In Alzheimer's disease, aggregates can occur both extracellular as neuritic plaques composed of Aβ peptide, and intracellular as neurofibrillary tangles of hyperphosphorylated TAU protein. In Amyotrophic lateral sclerosis (ALS) aggregation of ubiquitinated proteins, including FUS, TDP-43 and OPTN, occurs in degenerating motor neurons. Interestingly, these aggregated ubiquitinated proteins co-occur with perikaryal inclusions of neurofilament (NF). Abnormal NF aggregation and misassembly has also been reported in other motor neuron diseases such as giant axonal neuropathy (GAN) and Charcot-Marie-Tooth disease (CMT). However, the causes for NF accumulation appear to be heterogeneous and not fully understood.

### SUMMARY OF THE INVENTION

All ‘naturally’ occurring amyloidogenic protein pathology relies on expression of ‘aggregate-prone’ proteins from a given genome. Gene expression and protein translation are guided by well-known molecular mechanisms. We discovered in all vertebrate genomes, including human genomes, that specific non-coding DNA sequences downstream of regular termination codons contain sequence motives (cryptic amyloidogenic elements), which, after translation into proteins, act as effective amyloidogenic seeds (Rebelo et al., Am J Hum Genet (2016) 98(4):597-614). Described herein, is a specific type of such proteins originating from elongated translation of the original reading frame into the 3′-UTR or by switching into alternative reading frames that omit the natural termination codon and thus also read into the 3′-UTR until a new termination codon appears.

Such artificially forced expression of elongated, and now amyloidogenic, proteins will result in cellular aggregates and cause cancer cells to revert into a dormant state or enter apoptosis. Identified and described below are proteins that contain such elements, how they are to be confirmed as targets in vitro, and how the in vivo activation is possible, including DNA editing, such as using guided CRISPR/Cas9, and pharmacological induced read-through.

The invention provides various materials, methods, and uses relating to treatment of undesirable neoplastic cells, such as cancer cells. While amyloid aggregation is detrimental to nerve cell survival, it is of practical use to reduce viability of cancer cells as described herein. In particular, the present disclosure relates to materials and methods for treating or preventing the reoccurrence of cancer using toxic amyloidogenic aggregating proteins or fragments thereof. In particular, the specific amyloidogenic proteins in question are artificially created by incorporating specific downstream (3-UTR) amyloidogenic elements into an elongated protein isoform.

For example, one aspect of the invention is a method of treating a mammalian subject to inhibit growth of neoplastic cells. An exemplary method comprises:

administering to the subject a composition that comprises an agent selected from the group consisting of:
a. an agent that comprises an amyloidogenic peptide;
b. an agent that comprises a nucleotide sequence that encodes the amyloidogenic peptide; and
c. an agent that induces expression of an endogenous nucleotide sequence that encodes the amyloidogenic peptide.

Amyloidogenic peptides are peptides that have a propensity to fold into an improper or undesirable shape and form ordered aggregates referred to as fibrils. In preferred variations of the invention, the amyloidogenic peptide (relevant to either (a) or (b) or (c) above) is either:

(i) a Cryptic Amyloidogenic Element (“CAE”) encoded by nucleic acid in a 3′-untranslated region (“3′-UTR”) of a gene in the neoplastic cell; or

(ii) comprises an amino acid sequence that is at least 70% identical to the CAE and that retains the property of amyloidogenic aggregation.

In some variations, the amyloidogenic peptide has an amino acid sequence at least 75%, at least 80%, at least 85%, at least 90%, at least 95%, at least 97%, at least 98%, or at least 99% identical to the CAE. In some variations, the amyloidogenic peptide has an amino acid sequence identical to the CAE except at 1, 2, 3, 4, 5, 6, 7, 8, 9, or 10 amino acid positions. Preferred variants from the naturally occurring CAE differ with conservative substitutions and/or differ with substitutions that increase the propensity of the peptide to form toxic amyloid fibrils.

The term “Cryptic Amyloidogenic Element” refers to a peptide sequence that has amyloidogenic properties; that is encoded by nucleic acid in the genome of an organism; and that is not normally expressed in healthy cells because its coding sequence resides in an untranslated region of the genome, such as a 3′-untranslated region of a gene. A CAE that is located in a 3′ untranslated region is expressed when the gene is mutated to eliminate the natural (wild type) termination codon (e.g., missense mutation or frameshift mutation), or when the cell is subjected to an agent or condition that causes termination codon read-through during mRNA translation.

Aspects of the invention that are described or claims as methods can alternatively be described or claims as uses.

For instance, a related aspect of the invention is the use of a composition that comprises an agent to inhibit growth of neoplastic cells in a mammalian subject, wherein the agent is selected as described in the preceding paragraphs.

Similarly, another related aspect of the invention is the use of a composition that comprises an agent for the manufacture of a medicament to inhibit growth of neoplastic cells in a mammalian subject, wherein the agent is selected as described in the preceding paragraphs.

In some variations, the agent is administered in an amount effective to slow or halt the grow of the neoplastic cells. In some variations, the agent is administered in an amount effective to kill neoplastic cells in the subject.

A “mammalian subject” can be any mammal. Particularly contemplated include animals of agricultural importance, such as bovine, equine, and porcine animals; animals important as domestic pets, including canines and felines; animals important in research, including rodents and primates; large endangered species and zoo animals such as primates, felines, giraffes, elephants, rhinos. Especially contemplated are humans.

It is contemplated that the invention is applicable to the treatment of any neoplastic condition. In some variations, the neoplastic cells are malignant. Treatment of all cancers and tumor types are contemplated. In some variations, the neoplastic cells are from a cancer of neurogenic origin or a glia-derived tumor. For instance, treatment of each of neuroblastomas, central neurocytomas, and retinoblastomas is contemplated. For these particular neoplastic targets, it is specifically contemplated that the amyloidogenic peptide is encoded by the 3′-UTR of an NEFH or NEFL gene.

More generally, in some variations of the invention, the agent is selected based on an analysis of proteins that are determined to be, or determined to be likely to be, highly expressed in the target neoplastic cells.

Thus, in some variations a CAE is selected that is located in a gene that is highly expressed in the neoplastic cells, but not expressed or expressed at very low levels in normal cells. All variety of techniques are contemplated for determining genes that are highly expressed in the neoplastic cells. Some techniques analyze a sample of the neoplastic cells, e.g., from a biopsy, to provide highly personalized and targeted care for the individual. Other variations rely on data taken from similar neoplastic cells (e.g., from databases) or from healthy counterpart cells (data from the individual or from a database).

Thus, in some variations, the method or use of the invention further comprises additional steps of: screening the neoplastic cells to identify one or more genes that are highly expressed therein; and selecting, as the amyloidogenic peptide, a peptide that is encoded by a CAE that is located in the 3′-UTR of one of the highly expressed genes.

In some embodiments, this screening of the neoplastic cells comprises measuring protein and/or mRNA in a sample of the neoplastic cells. In other embodiments, the screening of the neoplastic cells comprises: pathological examination to characterize the neoplastic cells. e.g., to identify the type of cancer or tumor; and identifying genes that are highly expressed in a database of gene expression data for the neoplastic cell type, or for healthy cells of the type from which the neoplastic cells arose.

When one or more highly expressed genes are identified, the list can be screened for candidates having a CAE in the 3′-UTR. For example, in some variations, the selecting of the amyloidogenic peptide comprises analyzing the 3′-UTR of the genes identified as highly expressed; and selecting from that set of genes a gene having a 3′-UTR containing an open reading frame that encodes a peptide with amyloidogenic properties.

In some variations, the selecting of a gene having the CAE entails analysis of the 3′-UTR sequence of the mRNA (or cDNA) for evidence of amyloidogenic motifs, as described below in detail. However, such analysis has already been performed herein, and thousands of putative CAE's identified, as set forth below in Table 1. Thus, in some variations, the selecting of the amyloidogenic peptide comprises selected a gene from the list in Table 1 below that is among the genes identified as highly expressed in the neoplastic cells.

In some variations of the invention, the CAE is present in the 3′-UTR of a gene set forth in Table 1. More particularly, in some variations, the CAE is in the 3′-UTR of a gene selected from the group consisting of APOA2, ATXN2, B2M, FGB, FUS, IAPP, LYZ, NOTCH3, PRNP, RHO, SAA1, SNCA, NEFH, NEFL, BSCL2, MAPT, and TTR, as presented in Table 3, and also Table 4. Still more particularly, in some variations the CAE is in the 3′-UTR of a gene is selected from NEFH, NEFL, and FUS. Still more particularly, in some variations the amyloidogenic peptide comprises the amino acid sequence QFSLFLSL. Still more particularly, in some variations the amyloidogenic peptide comprises an amino acid sequence at least 70% identical (or at least 75%, 80%, 85%, 90%, 95%, or 100% identical) to the sequence SSRIRVTQFSLFLSLCKKKLLR, and retains the property of amyloidogenic aggregation.

In some variation of the invention the amyloidic element is in close proximity of an Arginine/Histidine-rich region of ˜20 bases. This region may serve as anchor for Amyloid body formation, a specific form of aggregation (Audas et al., Developmental Cell (2016) 39:155-168).

In some embodiments of the invention, the amyloidogenic peptide is introduced to the cell exogenously, by administering to the subject in need of the treatment either a composition comprising the peptide, or a composition comprising a nucleotide sequence that encodes the peptide and that is capable of being expressed in the cell.

Thus, in some variations, the agent of the method or use of the invention comprises a nucleotide sequence that encodes the amyloidogenic peptide. In some particular embodiments, the agent comprises cDNA or mRNA encoding the amyloidogenic peptide.

In embodiments where the agent is a nucleic acid, suitable expression control sequences (such as promoters, enhancers, polyadenylation signals sequences, etc.) are included in some variations, to promote expression of the encoded amyloidogenic peptide. Thus, in some variations, the agent further comprises a promoter operatively linked to the nucleotide sequence that encodes the amyloidogenic peptide. Optionally, the promoter is a tissue-selective promoter that promotes expression of genes in the neoplastic cells more readily than other cell types. In highly preferred embodiments, a tissue-specific promoter is selected. Fewer side-effects are expected with more selective promoters.

In some embodiments, the agent comprises a vector that comprises the nucleotide sequence that encodes the amyloidogenic peptide. All variety of gene therapy vectors are contemplated.

When the agent (nucleic acid or peptide) is administered exogenously, sequence variation from an endogenous CAE is possible. In some variations, the amyloidogenic peptide comprises an amino acid sequence at least 70% identical to the amino acid sequence of the CAE and retains the property of amyloidogenic aggregation. Greater degrees of similarity, e.g., at least 75%, 80%, 85%, 90%, 95%, or 100% identical, are also specifically contemplated, where the peptide retains amyloidogenic properties.

In other embodiments of the methods and uses of the invention, the selected agent is neither the amyloidogenic peptide or coding sequence per se, but rather, an agent which causes one or more endogenous CAE to be expressed in the target neoplastic cells. In some variations of this embodiment, the agent is a chemical agent that causes, induces, or promotes translation through natural stop codons, leading to translation of a gene product that includes the CAE located in the 3′-UTR. In other variations, the agent comprises one or more molecules that cause targeted genetic modification of the target cell. The targeted genetic modification can be a missense modification of a stop codon into a codon for an amino acid. The targeted genetic modification also can be an insertion or deletion in the stop codon or upstream of the stop codon that leads to a frameshift and translation of a modified protein that includes the CAE from the 3′-UTR of the wild type gene.

Thus, for example, in some embodiments of the method or use of the invention, the amyloidogenic peptide comprises the CAE, and the agent causes expression of an endogenous nucleotide sequence that encodes the CAE. For instance, in some variations, the agent causes expression by causing translation of mRNA beyond the regular stop codon of the gene. Exemplary agents that are specifically contemplated include an aminoglycoside antibiotic that induces translational read-through. Examples of cancer related genes suitable for this approach are given in Table 6—those genes contain the CAE in the first reading frame.

In some embodiments of the method or use of the invention, the agent induces expression of the amyloidogenic peptide by modifying chromosomal DNA of the neoplastic cells to create a modified gene that includes the CAE in-frame with the start codon, uninterrupted by a stop codon. A variety of tools now exist to achieve such targeted modification.

In some variations, the agent comprises a CRISPR Cas9 protein and one or more guide RNA molecules to introduce a site-specific modification of the chromosomal DNA to create a modified gene that includes the CAE in-frame with the start codon, uninterrupted by a stop codon. Optionally, the agent further includes a template nucleic acid for homology directed repair. In some variations, the agent comprises a zinc finger nuclease (ZFN) or a transcription activator-like effector nuclease (TALEN) to introduce a site-specific modification of the chromosomal DNA to create a modified gene that includes the CAE in-frame with the start codon, uninterrupted by a stop codon.

In some variations, chromosomal DNA is modified by eliminating the regular stop codon of the gene. In some variations, the chromosomal DNA is modified by changing the reading frame of the gene such that the CAE is in-frame with the start codon free of interruption by a stop codon.

As described herein in detail, many putative CAE elements have been identified, and in some variations of the invention, two or more are enlisted for treatment simultaneously. In some variations, two or all three of the following are employed: a. an agent that comprises an amyloidogenic peptide; b. an agent that comprises a nucleotide sequence that encodes the amyloidogenic peptide; and c. an agent that induces expression of an endogenous nucleotide sequence that encodes the amyloidogenic peptide. In other variations, two or more of the same category are employed.

CRISPR provides an especially appealing tool for inducing expression of two or more endogenous nucleotide sequences that encode amyloidogenic peptides, because a single CRISPR construct can be constructed to deliver Cas9 (gene or protein), two or more guide RNA's, and optionally two or more templates for homology directed repair.

All routes and vehicles for administration are contemplated. In some variations, the agent is formulated with one or more pharmaceutically acceptable carriers. In some variations, the agent is locally administered to a tumor that comprises the neoplastic cells. For example, if the tumor is in the eye or brain, then the agent is locally administered intracranially or intraocularly or intravitrially.

Still further embodiments of the invention include materials that are useful as therapeutic agents and/or useful for practicing methods of the invention.

For example, an embodiment of the invention is a polynucleotide comprising a nucleotide sequence that encodes an amyloidogenic peptide fused in-frame with an expression control sequence to promote expression of the amyloidogenic peptide in mammalian cells, wherein the amyloidogenic peptide is either:

(i) a Cryptic Amyloidogenic Element (“CAE”) encoded by nucleic acid in a 3′-untranslated region (“3′-UTR”) of a gene in the neoplastic cell; or

(ii) comprises an amino acid sequence at least 80% identical to the CAE and retains the property of amyloidogenic aggregation.

For this embodiment of the invention, particular embodiments are contemplated in which the CAE is present in the 3′-UTR of a genome-wide gene set forth in Table 1. More particularly, the putative CAE in the 3′-UTR of any of the following genes is specifically contemplated: APOA2, ATXN2, B2M, FGB, FUS, IAPP, LYZ, NOTCH3, PRNP, RHO, SAA1, SNCA, NEFH, NEFL, BSCL2, MAPT, and TTR. In very particular embodiments, the CAE is in the 3′-UTR of a gene is selected from NEFH, NEFL, and FUS. Known aggregation prone gene targets found to also encode non-coding CAE elements are listed in Table 3 and oncogene targets found to contain CAE elements are listed Table 4. These lists of genes are contemplated for use in the instant invention, as groups (genera), and individually. All subgroups from these tables are specifically contemplated as subgroups suitable for practicing the instant invention, as if each such subgroup were individually listed here.

In some variations, the invention includes an expression vector comprising any of the foregoing polynucleotides. In particular embodiments, the vector comprises a viral vector. Exemplary vectors that are specifically contemplated include retroviral, adenoviral, adeno-associated viral (AAV), and herpes simplex viral vectors.

In some variations, the invention is directed to a composition that comprises any of the foregoing polynucleotides or vectors and a pharmaceutically acceptable carrier.

Still additional aspects of the invention include CRISPR agents. For example, the invention includes a polynucleotide comprising a nucleotide sequence of a crRNA sequence operably linked to a tracrRNA sequence, wherein the crRNA sequence targets a 3′ end of a gene set forth in Table 1. In particular embodiments, the gene to be targeted is selected from the group consisting of APOA2, ATXN2, B2M, FGB, FUS, IAPP, LYZ, NOTCH3, PRNP, RHO, SAA1, SNCA, NEFH, NEFL, BSCL2, MAPT, and TTR. In highly specific embodiments, the gene is selected from NEFH, NEFL, and FUS.

In the context of oligonucleotides useful for directing CRISPR Cas9 nucleases to a sequence targeted for cleavage, a crRNA can be operably linked to a tracrRNA sequences in the manner that occurs in nature. Specifically, the two nucleotides in nature are made as separate strands which have segments that share reverse complement homology, such that one strand partially anneals to the other to form a double-stranded region held together by hydrogen bonds. Alternatively, crRNA and tracrRNA can be formed in a single polynucleotide strand, e.g., as described by Jinek et al., “A Programmable Dual-RNA—Guided DNA Endonuclease in Adaptive Bacterial Immunity,” Science 337: 816 (2012), incorporated herein by reference in its entirety. As described therein, essential features from crRNA and trRNA can be engineered into a single, chimeric RNA molecule, with the crRNA at the 5′ end and tracrRNA at the 3′ end. In some embodiments, a spacer, such as a GAAA tetraloop, is included between the 3′ end of the crRNA and the 5′ end of the tracrRNA. As described herein and in CRISPR scientific literature, the targeting function of the targeting domain of crRNA is manifested by near-perfect or perfect sequence complementarity to a selected target site in the genome. This region of complementarity is selected to be, for example, 15-40 nucleotides in length, and more preferably 15-30, 15-25, or 17-20 nucleotides in length. All integer lengths in these ranges are individually contemplated as embodiments of the invention.

In particular variations, the crRNA sequence targets a site upstream of, and within 100 nucleotides of, the natural stop codon of the gene, said site being immediately upstream of a protospacer adjacent motif (PAM) sequence of a Cas9 protein. Targeting within 90, or 80, or 70, or 60, or 50, or 40, or 30, or 20, or 10 nucleotides of the stop codon is specifically contemplated, as is targeting the stop codon itself.

Genes suitable for targeting with CRISPR Cas9 (or other genetic modification techniques) include those listed in Tables 1, 3, and 4, for example. Using the parameters described herein with respect to the type of modification desired, software tools exist for designing suitable targeting nucleic acids from model human genome sequences or, in some embodiments, from a subject's own sequenced genome. Exemplary targeting sequences for some of the genes identified herein are set forth in Table 5 and in SEQ ID NOs: 1-42. Thus, in some variations, the polynucleotide of the invention comprises a nucleotide targeting sequence which comprises or consists essentially of or consists of 17-20 nucleotides from a sequence set forth in any of SEQ ID NO. 1-42.

For polynucleotides described herein, sequences generally have been presented using A/G/C/T for adenine, guanine, cytosine, and thymine as is conventional IUPAC practice. However, as described herein and known in the art, CRISPR Cas9 operates as an RNA-driven nuclease, and natural RNA comprises U/uracil rather than T/thymine. The presentation of sequences with “T” should be understood in all cases to include and represent the equivalent RNA/“U” sequence, unless context unambiguously states otherwise. Both single stranded and double stranded polynucleotides are contemplated.

In some embodiments, the invention includes a polynucleotide comprising:

(a) a nucleotide sequence of a crRNA sequence, said crRNA sequence including:
(i) a nucleotide targeting sequence of 17-20 nucleotides that hybridizes to a 3′ end (“target site”) of a gene set forth in Table 1, and (ii) a nucleotide sequence that hybridizes to a tracrRNA recognized by a Cas9 protein, wherein (i) is disposed 5′ relative to (ii) and wherein the targeting sequence targets a site inclusive or upstream of, and within 100 nucleotides of, the wild type stop codon of the gene, said site being immediately upstream of a protospacer adjacent motif (PAM) sequence for said Cas9 protein; or (b) a nucleotide sequence encoding (a).

In some embodiments, the invention is a polynucleotide comprising:

(a) a nucleotide sequence of a CRISPR guide RNA including, from 5′ to 3′:
(i) a nucleotide targeting sequence of 17-20 nucleotides that hybridizes to a 3′ end (“target site”) of a gene set forth in Table 1, (ii) a linker sequence of 3-25 bases, such as a tetraloop sequence, and (iii) a nucleotide sequence of a tracrRNA recognized by a Cas9 protein, wherein the targeting sequence targets a site inclusive or upstream of, and within 100 nucleotides of, the wild type stop codon of the gene, said site being immediately upstream of a protospacer adjacent motif (PAM) sequence for said Cas9 protein; or (b) a nucleotide sequence encoding (a).

For these embodiments, the genes set forth in Table 3 or Table 4 are among the preferred genes around which to design a targeting crRNA sequence.

In some variations of the polynucleotide of the invention, the crRNA sequence targets a site upstream of, and within 100 nucleotides of, the natural stop codon of the gene, said site being immediately upstream of a protospacer adjacent motif (PAM) sequence for a Cas9 protein. As described herein, the target of course also can be the stop codon itself.

In still further variations, the invention includes a vector comprising the polynucleotide to be used as a CRISPR agent. Optionally, the vector further comprises a nucleotide sequence that encodes a Cas9 polypeptide that complexes with the tracrRNA sequence and that cleaves upstream of the PAM sequence. Optionally, the vector further comprises a nucleotide sequence of a crRNA sequence operably linked to a tracrRNA sequence, wherein the crRNA sequence targets a 3′ end of a second gene set forth in Table 1; three, four, or more are specifically contemplated.

More generally, in some variations, the invention is a vector comprising a nucleotide sequence that comprises or that encodes one or more polynucleotides of the invention. In some variations, the vector is an expression vector. For instance, in the expression vector, the nucleotide sequence that encodes the polynucleotide is operably linked to an expression control sequence (e.g., a promoter) to promote expression of the polynucleotide as RNA in a human cell. In some variations, the expression control sequence comprises a constitutive promoter. Exemplary promoters include the U6, CMV, EF1α, or CMV promoters.

In some variations, the expression vector is designed for use in a subject's cell, e.g., a human cell that is a neoplastic cell. In some variations, the cell has one or more genes that is highly expressed compared to a normal cell of the same cell type, and the expression control sequence that is selected for the vector comprises a promoter of one of the genes that is highly expressed. Differential expression between neoplastic versus control helps to focus the effects of the therapy on target neoplastic cells and reduce off-target side effects.

As described above, in some variations of the invention the crRNA and tracrRNA are fused as a synthetic single strand guide RNA. In other variations, the two functionalities are carried on separate strands. In the latter scenario, in some aspects of the invention where the expression vector that includes an expression cassette for the crRNA optionally further includes a promoter sequence operably linked to a nucleotide sequence encoding a tracrRNA.

In some variations, the expression vector according to the invention, further includes a promoter sequence operably linked to a nucleotide sequence encoding a Cas9 nuclease that complexes with tracr RNA that comprises the tracrRNA sequence to be used, and that cleaves upstream of the PAM sequence. In some preferred variations, the expression control sequence or coding sequence for the Cas9 nuclease further comprises a nuclear localization sequence (NLS).

Exemplary Cas9 nuclease for practice of the invention include those from Streptococcus pyogenes, Listeria innocua, Staphylococcus aureus, Streptococcus thermophiles, and synthetic variants derived therefrom. Scientists have extensively studied Cas9 biology and have now designed many variants, e.g., by introducing mutations in the primary amino acid sequence to modulate specificity, nuclease functioning, and the like. Such variants are contemplated. Likewise, Cas9 has now been characterized in many other species of bacteria, and all such Cas9 are suitable and contemplated for practice of aspects of the invention. In some variations, the nucleotide sequence encoding the Cas9 nuclease is a codon-optimized sequence. In particular, the codons are optimized for mammalian (e.g., human expression, based on species codon usages). For example codon optimized Cas9 coding sequences of proteins derived from Streptococcus pyogenes, Listeria innocua, Staphylococcus aureus, or Streptococcus thermophiles are contemplated.

In some variations of the invention, the expression vector of the invention further includes a nucleotide sequence encoding a DNA repair template operably connected to a promoter. For example, in some variations, the DNA repair template includes a sequence to introduce a missense or frameshift mutation at point of cleavage by the Cas9 via homology-directed repair (HDR), and includes segments flanking said sequence that are homologous to the predicted site of Cas9 cleavage. In some variations, the DNA repair template includes sequence to introduce a missense mutation in a stop codon of the gene, or a frameshift mutation in the coding sequence of the gene.

In some variations, the vector of the invention is a vector selected from the group consisting of a retroviral, adenoviral, adeno-associated viral (AAV), and herpes simplex viral vectors.

Other tools for delivering CRISPR/Cas9 reagents of the invention are contemplated as well. For example, in some variations, the invention includes a lipid nanoparticle comprising or containing a polynucleotide of the invention, or a vector of the invention.

In still further variations, the invention includes a composition comprising a polynucleotide or vector or a nanoparticle or other therapeutic agent described herein, and a pharmaceutically acceptable carrier.

Likewise, it should be appreciated that therapeutic agents described herein are suitable and intended for use in therapeutic method and uses described herein. Thus, still other aspects of the invention are any of the methods or uses that use an agent as described herein as aspects of the invention, wherein the agent comprises a polynucleotide, vector, nanoparticle, or composition as described herein.

Methods and materials of the invention can be combined with standard-of-care cancer therapies to achieve a therapeutic effect greater than either approach on its own. Thus, in some variations, methods and uses of the invention further comprise administration of a standard of care therapy before, during, or after the method or use described above. Kits and combinations of agents are likewise contemplated as materials of the invention.

Aspects of the invention that have been described herein as methods also can be described as “uses,” and all such uses are contemplated as aspects of the invention. Likewise, compositions described herein as having a “use” can alternatively be described as processes or methods of using, which are contemplated as aspects of the invention. It should be understood that details described herein in the context of describing a method are applicable to “uses,” and vice versa.

Reference throughout this specification to “one embodiment”, “some embodiments” or “an embodiment” means that a particular feature, structure or characteristic described in connection with the embodiment is included in at least one embodiment of the present disclosure. The particular features, structures, or characteristics described herein may be combined in any suitable manner, and all such combinations are contemplated as aspects of the invention.

Unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

The invention includes, as an additional aspect, all embodiments of the invention narrower in scope in any way than the variations defined by specific paragraphs above. For example, where certain aspects of the invention that are described as a genus or set, it should be understood that every member of a genus or set is, individually, an aspect of the invention. Likewise, every individual subset is intended as an aspect of the invention. By way of example, if an aspect of the invention is described as a members selected from the group consisting of 1, 2, 3, and 4, then subgroups (e.g., members selected from {1,2,3} or {1,2,4} or {2,3,4} or {1,2} or {1,3} or {1,4} or {2,3} or {2,4} or {3,4}) are contemplated and each individual species {1} or {2} or {3} or {4} is contemplated as an aspect or variation of the invention. Likewise, if an aspect of the invention is characterized as a range, such as a temperature range, then integer sub-ranges are contemplated as aspects or variations of the invention.

The headings herein are for the convenience of the reader and not intended to be limiting. Additional aspects, embodiments, and variations of the invention will be apparent from the Detailed Description and/or Drawing and/or claims.

Although the Applicant invented the full scope of the invention described herein, the Applicant does not intend to claim subject matter described in the prior art work of others. Therefore, in the event that statutory prior art within the scope of a claim is brought to the attention of the Applicants by a Patent Office or other entity or individual, the Applicant reserves the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIG. 1 shows NEFH protein domains and mutations associated with diseases. Coding KSP deletions and insertions from reported ALS cases represented by triangles. CMT frameshift variants from UK1 and F2 are pointed by the arrows.

FIGS. 2A-2D shows the identification of cryptic amyloidogenic elements (CAEs) in the NEFH and NEFL-3′UTRs. (FIG. 2A) Clustal Omega multiple-sequence alignment of wild-type NEFH (SEQ ID NO. 43) and frameshift variants harbored by the CMT-affected families (ASP1004Glnfs*56: SEQ ID NO. 44; Pro1008Alafs*54: SEQ ID NO. 45). Translation of the NEFH-3′ UTR open reading frames (ORFs) for ORF1 (SEQ ID NO. 46), ORF2 (SEQ ID NO. 47), and ORF3 (SEQ ID NO. 48) are illustrated. (FIG. 2B) Consensus sequence of positive residues (asterisks) for all aggregation predictors tested for NEFH 3′-UTR ORF3 (SEQ ID NO. 48). (FIG. 2C) TANGO score of NEFL 3′-UTR ORF1 (SEQ ID NO. 49), ORF2 (SEQ ID NO. 50), and ORF3 (SEQ ID NO. 51). (FIG. 2D) Consensus sequence of positive residues (asterisks) for all aggregation predictors tested for NEFL 3′-UTR ORF1 (SEQ ID NO. 49).

FIG. 3 shows NEFH Co-immunuoprecipitation: Cell lysates were immunoprecipitated with an antibody against either GFP or Myc and IgG as a negative control. Both GFP-WT-NEFH and GFP-FS-NEFH were co-immunoprecipitated with NEFL-myc. Kinesin was also present in the co-immunoprecipitate.

FIG. 4 shows confocal microscopy time lapse images of Neuro-2a cells expressing NEFH-CAE. Cells lose neuronal-like projection and adopt a more rounded shape before detaching from plate.

FIG. 5 shows effect of NEFH-CAE expression in vivo in vertebrate model Zebrafish embryos injected with RNA encoding GFP-NEFH-CAE do not show major morphological defects at 48 hpf. Quantification of the average axon length shows a decrease in GFP-FS-NEFH injected fishes compared to both GFP-WT-NEFH injected and uninjected larvae. Axon length is not significantly different between GFP-WT-NEFH injected fishes and uninjected control fish. The average axon length per fish was calculated from the first four myotomes. Data was compiled from three independent experiments and significance was determined by a one-way ANOVA with Bonferroni correction test. P-values are *0.024 and **0.014.

### DETAILED DESCRIPTION

### Definitions

The terminology used herein is for the purpose of describing particular embodiments only and is not intended to be limiting.

“Cryptic amyloidogenic element (CAE)” as used herein in, refers to a stretch of amino acids, of any length or combination, predicted to self-aggregate and/or induce amyloid aggregation and proteotoxicity in a target cell, and that has the additional properties summarized in [0009].

“Fragment” as used herein in the context a of an amyloidogenic peptide or its corresponding coding sequence means a portion of a reference peptide or polypeptide or nucleic acid sequence that retains a pertinent biological property (e.g. a portion that is effective to aggregate or directly or indirectly induce amyloidogenic peptide aggregation and proteotoxicity).

“Frameshift mutation” as used herein, refers to a genetic mutation caused by indels (insertions or deletions) of a number of nucleotides in a DNA sequence that is not divisible by three and that results in a change of codon reading frame of a sequence of nucleic acids. As described herein, of particular interest are frameshift mutations that result in translation read-though of a wild type stop codon.

“Guide RNA” as used herein, refers to the polynucleotide sequence that contains a targeting sequence (also referred to as guide sequence or CRISPR RNA sequence [crRNA]) that specifies the target site and the trans-activating CRISPR [tracrRNA] or tracr sequence.

“3′-UTR” as used herein refers to the 3′ untranslated region (UTR) of a gene of interest. UTRs of a gene are transcribed but not translated. The 3′-UTR starts immediately following the natural or wild type stop codon and continues until the transcriptional termination signal. For the purposes of this invention, 3′-UTR includes portions of a complete 3′-UTR that includes a CAE.

“Peptide” or “polypeptide” as used herein, refers to a linked sequence of amino acids and may be natural, synthetic, or a modification or combination of natural and synthetic.

“TANGO” is a statistical mechanics algorithm, to predict protein aggregation (Fernandez-Escamilla et al., Nat Biotechnol. 2004 22(10):1302-6, incorporated by reference in its entirety).

“Treating,” “treatment,” or “to treat” each refer to administration of a therapeutic agent or process to a mammalian subject to alleviate, suppress, repress, eliminate or slow the appearance of symptoms, clinical signs, or underlying pathology of cancer/tumor on a temporary or permanent basis. Successful prevention is demonstrated by a statistically significant reduction of occurrence in a population study over a finite and clinically meaningful period of time. In the context of an individual, prevention occurs if the cancer/tumor does not occur over a measurable period of time (e.g. weeks or months) following administration of an agent shown to have prevention properties in a population study. Repressing the cancer involves administering an agent of the present invention to a subject after clinical appearance of the disease resulting in a measurable and statistically significant reduction in growth and/or size of a tumor. Slowing the progression of disease, as measurable by a statistically significant reduction of occurrence in a population study over a finite and clinically meaningful period of time, is indicative of an agent useful to treat.

A “biological sample” from an organism comprises any tissue, cell, or fluid which can be analyzed for a trait of interest, such as nucleic acid. A “nucleic acid sample” refers to a biological sample obtained from an individual that contains nucleic acid (DNA or RNA).

The disclosed methods are useful for treating cancer, for example, inhibiting cancer growth, including complete cancer remission, for inhibiting cancer metastasis, and for promoting cancer resistance. The term “cancer growth” generally refers to any one of a number of indices that suggest change within the cancer to a more developed form. Thus, indices for measuring an inhibition of cancer growth include but are not limited to a decrease in cancer cell survival, a decrease in tumor volume or morphology (for example, as determined using computed tomographic (CT), sonography, or other imaging method), a delayed or slowed tumor growth, a destruction of tumor vasculature, an increase in the activity of cytolytic T-lymphocytes, and a decrease in levels of tumor-specific antigens.

The term “therapeutically effective amount” refers to an amount of a compound sufficient exhibit a detectable treatment, amelioration, or inhibitory effect in a patient or in an experimental trial involving multiple patients compared to a placebo or control. In the context of a therapy for a subject with a neoplastic condition, evidence of a therapeutic effect includes shrinkage of the neoplasm, a slowing or halting of growth/progression, inhibiting metastasis, increased survival, increased progression-free survival, increased quality of life during period of survival (e.g., reduction of symptoms/discomfort), and other accepted measures.

A dose of administration will depend on factors such as route of administration (local vs. systemic), patient characteristics (e.g., gender, weight, health, side effects); the nature and extent of the condition; and the therapeutic or combination of therapeutics selected for administration. Therapeutically effective amounts for a given situation can be estimated from in vitro studies to determine, e.g., IC50 concentrations, pre-clinical studies and clinical studies in animals and humans, and the like, and determined by routine experimentation that is within the skill and judgment of the clinician.

Abnormal protein aggregation is observed in an expanding number of neurodegenerative diseases. The origins of these aggregates are divers; however, they share similar structures and overlapping mechanisms of cellular toxicity in different diseases. Protein aggregates usually adopt high-ordered beta sheet quaternary structures forming insoluble fibrils termed amyloids (Aguzzi et al., Nature reviews Drug discovery (2010) 9:237-248). Disclosed herein is a mechanism of treating and/or preventing the reoccurrence of cancer by using intracellular toxic amyloidogenic peptide aggregation induced by a mutation event, such as a mutation characterized herein, in axonal neuropathy families.

The inventors have determined that previously unrecognized cryptic amyloidogenic elements (CAE) in the 3′-UTR of NEFH are expressed under certain circumstances (e g familial mutation), leading to aggregation and neuronal degeneration in certain familial neurodegenerative diseases. Described herein is the use of these amyloidogenic elements, including nucleic acids that encode them, to inhibit the growth and survival of cancerous cells. Specific frameshift variants in NEFH in CMT2 families result in stop-loss and translation of a CAE in the 3′-UTR.

Expression of the mutant NEFH resulted in prominent abnormal protein aggregates, disruption of the NF network, and altered cell dynamics. Interestingly, the same aggregation phenomenon was induced by triggering the translation of the 3′-UTR of NEFL and FUS. In vivo and in vitro results show that translation of CAE in the 3′-UTR of NFs cause axonopathy and could be of broader impact for neurodegenerative diseases.

### Genome Editing Techniques

Aspects of the invention involve the administration of an agent or agents to modify the genome of mammalian cells. In some embodiments, these agents can be used to induce the endogenous expression of CAEs.

Genome-editing is a method of genetic engineering in which DNA is inserted, deleted or replaced in the genome of an organism using engineered nucleases. Genome-editing techniques such as designer zinc fingers, transcription activator-like effectors nucleases (TALENs), or CRISPR-Cas (clustered regularly interspaced short palindromic repeats-CRISPR associated) systems are contemplated for producing targeted genome modification.

Genome editing may be used to induce missense or frameshift mutations in target genomic loci by single base modification (single nucleotide base change, insertion, or deletion) or by insertions or deletions of a number of nucleotides in a DNA sequence that is not divisible by three, respectively. A frameshift mutation will, in general, cause the reading of the codons after the mutation to code for different amino acids. In some embodiments, missense or frameshift mutations are used to eliminate stop codons. For example, a gene is modified to mutate a stop codon or introduce a change in reading frame, leading to translation of a CAE in the 3′-UTR of the target gene.

Aspects of the invention involve targeted genetic modification of cells. A variety of techniques are suitable including Zinc-finger nucleases (ZFNs) and TALENs and CRISPR-Cas systems.

### Zinc-Finger Nucleases (ZFNs) and Transcription Activator-Like Effector Nucleases (TALENs)

Zinc-fingers nucleases (ZFNs) and Transcription activator-like effector nucleases (TALENs) are customizable DNA-binding proteins that comprise DNA-modifying enzymes. Both can be designed and targeted to specific sequences in a variety of organisms (Esvelt and Wang, Mol Syst Biol. (2013) 9: 641, which is incorporated by reference in its entirety). ZFNs and TALENs can be used to introduce a broad range of genetic modifications by inducing DNA double-strand breaks that stimulate error-prone nonhomologous end joining or homology-directed repair at specific genomic locations. The versatility of ZFNs and TALENs arises from the ability to customize the DNA-binding domain to recognize virtually any sequence. These DNA-binding modules can be combined with numerous effector domains to affect genomic structure and function, including nucleases, transcriptional activators and repressors, recombinases, transposases, DNA and histone methyltransferases, and histone acetyltransferases. Thus, the ability to execute genetic alterations depends largely on the DNA-binding specificity and affinity of designed zinc finger and TALE proteins (Gaj et al., Trends in Biotechnology, (2013) 31(7):397-405). The following U.S. granted patents, incorporated by reference, describe the use of ZFNs and TALENs in mammalian cells, U.S. Pat. Nos. 8,685,737 and 8,697,853.

### CRISPR

CRISPR-Cas (clustered regularly interspaced short palindromic repeats-CRISPR associated) is an RNA-mediated adaptive immune system found in bacteria and archaea, which provides adaptive immunity against foreign nucleic acids (Wiedenheft et al., Nature (2012) 482:331-8; Jinek et al., Science (2012) 337:816-21). However, multiple research groups have demonstrated how the biological components of this system can be harnessed to introduce directed modification to the genome of mammalian cells (Cong et al., Science (2013) 339:819-23; Mali et al., Science (2013) 339(6121):823-6; Jinek et al., Elife. (2013); 2:e00471). CRISPR-Cas systems are generally defined by a genomic locus called the CRISPR array, a series of 20-50 base-pair (bp) direct repeats separated by unique “spacers” of similar length and preceded by an AT-rich “leader” sequence (Wright et al., Cell (2016) 164:29-44).

Three types of CRISPR/Cas systems exist, type I, II and III. The Type II CRISPR-Cas systems require a single protein, Cas9, to catalyze DNA cleavage (Sapranauskas et al., Nucleic Acids Res. (2011) 39(21): 9275-9282). Cas9 serves as an RNA-guided DNA endonuclease. Cas9 generates blunt double-strand breaks (DSBs) at sites defined by a 20-nucleotide guide sequence (also known as targeting nucleotide sequence or protospacer) which are homologous to target sequences in a genome of interest. The guide sequences are contained within an associated CRISPR RNA (crRNA) transcript. Cas9 can be programmed to cleave double-stranded DNA at any site defined by the guide RNA sequence and next to the protospacer-adjacent (PAM) motif, a short sequence required by and recognized by the CRISPR complex (Sapranauskas et al., Nucleic Acids Res. (2011) 39(21): 9275-9282; Jinek et al., Science (2012) 337:816-21). The precise sequence and length requirements for the PAM differ depending on the Cas9 (or other Cas enzyme) ortholog used, but PAMs are typically 2-5 base pair sequences adjacent the protospacer (that is, the target sequence). For example, the PAM sequence 5′ NNAGAAW 3′ is required for Streptococcus pyogenes Cas9 target binding, the PAM sequence 5′ NNGRRT 3′ or 5′ NNGRR(N) 3′ are required for Staphylococcus aureus Cas9 target binding, the PAM sequence 5′ NNGRRT 3 is required for Streptococcus thermophilus Cas9 target binding. Cas9 requires both the guide crRNA and a trans-activating crRNA (tracrRNA) that is partially complementary to the crRNA for site-specific DNA recognition and cleavage (Jinek et al., Science (2012) 337:816-21). The mature crRNA that is base-paired to tracrRNA forms a two-RNA structure that directs the CRISPR-associated protein Cas9 to introduce DSBs in target DNA. Tracr sequences for use with type II CRISPR-Cas systems are described in Jinek et al., Science (2012) 337:816-21 and Chylinski et al., RNA Biol (2013) 10(5): 726-737 which are incorporated by reference in their entirety, and specifically for their teachings regarding crRNA and Tracr sequences and guide RNAs referenced in the following paragraph.

Recent experiments showed that the crRNA:tracrRNA complex can be synthesized as a single transcript (single-guide RNA or sgRNA or chimeric guide RNA) encompassing the features required for both Cas9 binding and DNA target site recognition. Sequences of suitable sgRNAs are described in Jinek et al., Science (2012) 337:816-21 and are adaptable to the instant invention. Using sgRNA, Cas9 from Streptococcus pyogenes can be programmed to cleave double-stranded DNA at any site defined by the guide RNA sequence and including a GG protospacer-adjacent (PAM) motif (Sapranauskas et al., Nucleic Acids Res. (2011) 39(21): 9275-9282; Jinek et al., Science (2012) 337:816-21). Cas9 from other bacterial species utilize alternative PAM sequences, thereby increasing the number of CRISPR-targetable loci. The DSBs instigate either non-homologous end-joining (NHEJ), which is error-prone and conducive to frameshift mutations that knock out gene alleles, or homology-directed repair (HDR), which can be exploited with the use of an exogenously introduced double-strand or single-strand DNA repair template to knock in or correct a mutation in the genome. Therefore, in the presence of a homologous repair donor, the CRISPR/Cas9 system may be used to generate precise and defined modifications and insertions at a targeted locus through the HDR process. In some variations, the cell targeted to undergo the RNA-guided, site-specific cleavage of DNA will also be given a DNA repair template. For example, the donor DNA repair template that has the desired modification (e.g., missense codon change) or insertion (e.g., frameshift) is flanked by segments of DNA homologous to the blunt ends of the cleaved DNA (Mali et al., Science (2013) 33: 823-826). In some variations of the instant invention, the donor DNA repair template encodes a missense modification of a stop codon into a codon for a translated amino acid or it encodes an insertion in the stop codon or upstream of the stop codon that leads to a frameshift mutation and translation of a modified protein that includes the CAE from the 3′-UTR of the wild type gene. In still another variation, the donor DNA repair template encodes a toxic amyloidogenic peptide sequence that will be translated along with the native amino acid sequence of a highly expressed protein.

In the absence of a homologous repair donor, single DSBs generated by CRISPR/Cas9 are repaired through the error-prone NHEJ, which sometimes results in insertion or deletion (indel) mutations. Indel mutations in coding exons may introduce premature stop codons or frame-shift mutations, thereby inactivating the corresponding proteins.

The ability to easily target specific gene loci make CRISPR a useful tool for introducing genetic modifications for the invention, for example, to promote transcription of the CAE in the 3′-UTR of target genes.

Other publications describing the CRISPR systems and Cas9, include the following Cong et al. Science (2013) 339:819-23; Jinek et al., Elife. (2013) 2:e00471; Lei et al. Cell (2013) 152: 1173-1183; Gilbert et al. Cell (2013) 154:442-51; Lei et al. Elife (2014) 3:e04766; Perez-Pinela et al. Nat Methods (2013) 10: 973-976; Maider et al. Nature Methods (2013) 10, 977-979 which are incorporated by reference. The following U.S. and international patents and patent applications describe the methods of use of CRISPR, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 2014/0068797; and WO 2014/197568, all incorporated by reference in their entirety.

The CRISPR related protein, Cas9, can be from any number of species including but not limited to Streptococcus pyogenes, Listeria innocua, Staphylococcus aureus and Streptococcus thermophiles and optionally codon-optimized to express in the target cell of interest. The nucleotide sequences of Cas9 orthologs are described in the art, for example, in international patent application WO 2015048577A2 and US patent application 2014/0068797A1.

In one aspect, the invention uses CRISPR-Cas system comprising a Cas9 protein and one or more guide RNAs and optionally repair templates to modify eukaryotic genes that contain a CAE in the 3′-UTR of the gene. Exemplary genes are identified in Table 1. In some variations, CRISPR-Cas systems are used to modify NEFH or NEFL genes in a eukaryotic cell so that the 3′-UTR of NEFH or NEFL are translated in vivo.

The CRISPR-Cas reagents (e.g. CRISPR-Cas9) used to modify the genes are themselves an aspect of the invention. For example, the invention includes a guide RNA molecule, e.g., an isolated or non-naturally occurring gRNA molecule, comprising a targeting domain which is complementary with a target domain from the NEFH or NEFL genes. Particularly complemented are targeting domains including or just upstream of the stop-codon.

In one aspect, the invention uses CRISPR-Cas reagents (e.g. CRISPR-Cas9 and induce DSBs, NHEJ and frameshift mutation(s) in eukaryotic genes that contain a CAE in the 3′-UTR of the gene. Exemplary genes are identified in Table 1. In some variations, the exemplary genes identified in Table 3 or in Table 4, or subsets of those genes, are selected.

It will be understood from these Tables and from the description herein that the nature of the modification differs slightly, depending on the reading frame of a CAE. If a CAE falls in the same reading frame as the wild type protein (frame 1), then a missense or deletion of the stop codon achieves read-through. If a CAE falls in a different reading frame (frame 2 or frame 3) then a frameshift mutation achieves read-through. In some variations the frameshift occurs with a stop codon mutation. In some variations, a frameshift is introduced upstream of the stop codon, preferably in the exon that encodes the stop codon and/or the amino acids immediately upstream of the stop codon.

In another aspect, the invention uses CRISPR-Cas reagents (e.g. CRISPR-Cas9 homologous repair donor) induce DSBs, HDR and modification and insertion of nucleotide bases of stop codons in target eukaryotic genes that contain a CAE in the 3′-UTR of the gene. Exemplary genes are identified in Table 1.

In another aspect, CRISPR-Cas reagents (e.g. CRISPR-Cas9) are designed and made and used to alter the non-coding sequence of any gene listed in Table 1 to alter the expression of a CAE located in the 3′-UTR of the gene by targeting, e.g., a promoter, an enhancer, an intron, 3′UTR, and/or polyadenylation signal.

The agent may encode the wild-type sequence of the CRISPR related protein or a variant CRISPR related protein. The agent can include either the wild-type codon usage or codon usage optimized for a particular application, e.g., human codon optimization for human therapeutics. The type II CRISPR Cas9 systems are particularly preferred.

In some embodiments, DNA encoding Cas9 molecules (e.g., eaCas9 molecules) and/or gRNA molecules, are delivered into cells by art-known methods or as described herein. For example, Cas9-encoding and/or gRNA-encoding DNA are delivered, e.g., by vectors (e.g., viral or non-viral vectors), non-vector based methods (e.g., using naked DNA or DNA complexes), or a combination thereof.

Preferably, the vector is a viral vector, such as a lenti- or baculo- or preferably adeno-viral/adeno-associated viral vectors (AAV), but other means of delivery are known (such as yeast systems, macrovesicles, gene guns/means of attaching vectors to gold nanoparticles) and are provided. In some embodiments, one or more of the viral or plasmid vectors may be delivered via liposomes, nanoparticles, exosomes, macrovesicles, hydrodynamic-based gene delivery or a gene-gun. Cas9 and gRNA can be present in a single lentiviral transfer vector or separate transfer vectors. Adenoviral delivery of the CRISPR/Cas9 system is described in Holkers et al., Nature Methods (2014), 11(10):1051-1057 which is incorporated by reference in its entirety.

In certain embodiments the invention provides a method of treating cancer in a subject (e.g., mammal or human) or a non-human subject (e.g., mammal) in need thereof comprising modifying the subject or a non-human subject by manipulation of the target sequence comprising providing treatment comprising: delivering a non-naturally occurring or engineered composition comprising an AAV or lentivirus vector system comprising one or more AAV or lentivirus vectors operably encoding a composition for expression thereof, wherein the target sequence is manipulated by the composition when expressed.

In some embodiments, CRISPR/Cas9 multiplexing may be used to target multiple genomic loci wherein 2 or more guide RNAs are expressed as described in CRISPR 101: A Desktop Resource (1^{st} Edition), Addgene, January 2016 which is incorporated by reference in its entirety.

### Gene Expression Analysis

In an embodiment of the present invention, gene expression data are used to determine genomic targets. Expression data in the database derived from cells as closely related to the neoplastic cells of interest is preferably selected. Examples of databases include, but not limited to, Genbank public domain database available on the Internet at www.ncbi.nlm.nih.gov/Genbank; The Cancer Genome Atlas (http://cancergenome.nih.gov/); Gene Expression across Normal and Tumor tissue (GENT) database (http://medicalgenome.kribb.re.kr/GENT/); Achilles Project (http://www.broadinstitute.org/achilles); Cancer Cell Line Factory (CCLF) (www.broadinstitute.org/cellfactory); Connectivity Map (CMap) (http://www.broadinstitute.org/cmap).

In an alternative embodiment of the present invention, gene expression analysis of cells from a patient's tumor(s) to determine a molecular signature is performed using methods described in the art (Ramaswamy et al., PNAS (2001) 98: 15149-15154; Ramaswamy et al., Nat Genet. (2003) 33(1):49-54; Fehrmann et al., Nature Genetics (2015) 47, 115-125, which are incorporated by reference. Exemplary analyses include mRNA measurement and/or protein measurements and/or protein activity measurements.

### Gene Expression Constructs

In some embodiments of the present invention, gene expression constructs such as promoter expression plasmids are used to transform target neoplastic cells in vivo to express amyloidogenic peptides. The constructs described herein include a promoter (e.g. cytomegalovirus (CMV) promoter), the coding and or non-coding (e.g. 3′-UTR) regions from multiple genes encoding one or more wildtype or modified proteins (e.g. mutated), inteins, transcription termination or regulator elements sequences. In some embodiments tissue specific promoters may be used. For example, the Cre-loxP system may be utilized to express amyloidogenic peptides in specific tissues. In some cases the Cre-loxP gene expression construct is inducible.

In some embodiments, a target cell is screened to identify a gene that is highly expressed, and a promoter for the highly expressed gene is selected as a promoter for the gene expression construct. In some embodiments, a target neoplastic cell and corresponding healthy cells are screened to identify a gene that is highly expressed in the neoplastic cells but not highly expressed in the healthy control cells, and the promoter for such a gene is selected for the gene expression construct.

### Identification of Cryptic Amyloidogenic Elements (CAEs)

In an embodiment of the present invention, potential cryptic amyloidogenic elements (CAEs) are identified using in silico analysis. Bioinformatics aggregation prediction analysis of all 3′-UTR sequences in a genome of interest is performed using RefSeq transcripts from the National Center for Biotechnology Information (NCBI) database and aggregation prediction programs, TANGO and PASTA. Described in detail in example 6.

### Methods of Treatment

Provided herein is a method of inhibiting or reducing the growth, progression and/or reoccurrence of cancer.

Although more than one route can be used to administer an agent of the present invention, a particular route can provide a more immediate and more effective reaction than another route. Depending on the circumstances, a pharmaceutical composition comprising the agent is applied or introduced into body cavities, absorbed through the skin or mucous membranes, ingested, inhaled, and/or introduced into circulation. For example, in certain circumstances, it will be desirable to deliver the pharmaceutical composition orally; through injection or infusion by intravenous, intratumoral, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means; by controlled, delayed, sustained or otherwise modified release systems; or by implantation devices. In one aspect, drug exposure can be optimized by maintaining constant drug plasma concentrations over time. Such a steady-state is generally accomplished in clinical settings by continuous drug infusion at doses depending on the drug clearance and the plasma concentration to be sustained. If desired, the composition is administered regionally via intratumoral, administration, intrathecal administration, intracerebral (intra-parenchymal) administration, intracerebroventricular administration, or intraarterial or intravenous administration targeting the region of interest. Alternatively, the antibiotics, peptides and/or induction agents are administered locally via implantation of a matrix, membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

In some preferred embodiments involving genetic modification, the polynucleotide guide sequences and Cas9 polynucleotide or polypeptide sequence described herein are administered using adeno-associated virus (AAV) vectors or lipid nanoparticles. AAV-mediated CRISPR delivery is described in Wang et al., Int J Mol Sci. 17(5): 626 (2016); Gaj and Schaffer, Cold Spring Harb Protoc (11):086868 (2016). Lipid nanoparticle-mediated CRISPR delivery is described Zuris et al., Nature Biotechnology 33, 73-80 (2015); Yin et al., Nature Biotechnology 34, 328-333 (2016); Wang et al., Int J Mol Sci. 17(5): 626 (2016) which are incorporated by reference.

### Aminoglycoside Antibiotics

In some variations of the invention, a composition is administered to a subject in need of treatment, wherein the composition includes an agent that decreases translational fidelity and results in increased read-through of stop codons. Exemplary agents of this class include aminoglycoside antibiotics. See, e.g., Heier et al., “Translational readthrough by the aminoglycoside geneticin (G418) modulates SMN stability in vitro and improves motor function in SMA mice in vivo,” Hum Mol Genet. 2009 Apr. 1; 18(7): 1310-1322; 27; Stephenson J. “Antibiotics show promise as therapy for genetic disorders,” J. Am. Med. Assoc. 2001; 285:2067-2068; Zingman et al., “Aminoglycoside-induced translational read-through in disease: overcoming nonsense mutations by pharmacogenetic therapy,” Clin. Pharmacol. Ther. 2007; 81:99-103; Bidou et al., “Sense from nonsense: therapies for premature stop codon diseases,” Trends Mol Med. 2012 November; 18(11):679-88; Nudelman et al., “Redesign of aminoglycosides for treatment of human genetic diseases caused by premature stop mutations,” Bioorg Med Chem Lett. 2006 Dec. 15; 16(24):6310-5; Keeling et al., “Therapeutics based on stop codon readthrough,” Annu Rev Genomics Hum Genet. 2014; 15:371-94; and Gomez-Grau et al., “Evaluation of Aminoglycoside and Non-Aminoglycoside Compounds for Stop-Codon Readthrough Therapy in Four Lysosomal Storage Diseases,” PLoS One 10(8):e0135873 (2015), all incorporated herein by reference in their entirety, and specifically for their descriptions of compounds that promote stop-codon read-through and how to use them.

Exemplary agents that are specifically contemplated include an aminoglycoside antibiotic that increases or induces translational read-through in cells exposed to the antibiotic Aminoglycoside antibiotics or aminoglycosides exhibit concentration-dependent bactericidal activity. They mode of action includes binding to the 30S ribosome to inhibit bacterial protein synthesis. Examples of aminoglycoside antibiotics for use in the instant invention but not limited to, include the following: paromomycin, tobramycin, gentamicin, amikacin, kanamycin, neomycin, dibekacin, sisomicin, netilmicin and streptomycin.

### Formulation

In some variations, gene editing is contemplated as a method to activate CAE in 3′-UTRs. Any standard gene editing approach for in vivo applications that is able to introduce small insertions and deletions at a targeted locus or provide precise nucleotide exchanges will suffice. Typically, AAV particles from small scale and large scale AAV [symbol] packaging is applied directly for in vivo animal injection. The recommended titer for in vivo animal injection is 10¹¹ GC per mouse or 2×10⁹ GC/g (body weight). For local injections (e.g. into brain) a smaller quantity of AAV viruses can be applied.

In some variations agents of the present invention, e g, aminoglycoside antibiotics are formulated as a composition comprising one or more pharmaceutically acceptable carriers. The phrase “pharmaceutically (or pharmacologically) acceptable” refers to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

Suitable methods of administering a physiologically-acceptable composition, such as a pharmaceutical composition comprising a compound and/or micelle described herein, are well known in the art.

### Combination Therapy

It is contemplated the two or more agents of the present invention may be given simultaneously, in the same formulation. It is further contemplated that the two or more agents are administered in separate formulations and administered either separately or concurrently, with concurrently referring to agents given within 30 minutes of each other.

The agents of the invention may be administered as a monotherapy or simultaneously or metronomically with other treatments, which may be a surgery or removal of a tumor. The term “simultaneous” or “simultaneously” as used herein, means that the two treatments are administered within 48 hours (h), preferably 24 h, more preferably 12 h, yet more preferably 6 h, and most preferably 3 h or less, of each other. The term “metronomically” as used herein means the administration of the agent at times different from the other treatment and at a certain frequency relative to repeat administration.

Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In some variations of the invention, agent of the invention is administered as a co-therapy in combination with a standard-of-care therapy for a neoplastic condition. Exemplary standard-of-care anti-neoplastic therapies include antitumor agents and chemotherapeutic agents such as an aromatase inhibitor, an anti-estrogen, an anti-androgen, a gonadorelin agonist, a topoisomerase I inhibitor, a topoisomerase II inhibitor, a microtubule active agent, an alkylating agent, a retinoid, a carotenoid, a tocopherol, a cyclooxygenase inhibitor, an MMP inhibitor, a mTOR inhibitor, an antimetabolite, a platin compound, a methionine aminopeptidase inhibitor, a bisphosphonate, an antiproliferative antibody, a heparanase inhibitor, an inhibitor of Ras oncogenic isoforms, a telomerase inhibitor, a proteasome inhibitor, a compound used in the treatment of hematologic malignancies, a Flt-3 inhibitor, an Hsp90 inhibitor, a kinesin spindle protein inhibitor, a MEK inhibitor, an antitumor antibiotic, a nitrosourea, a compound targeting/decreasing protein or lipid kinase activity, a compound targeting/decreasing protein or lipid phosphatase activity, any further anti-angiogenic compound, and combinations thereof. Specific examples of antitumor agents include, but are not limited to, azacitidine, axathioprine, bevacizumab, bleomycin, capecitabine, carboplatin, chlorabucil, cisplatin, cyclophosphamide, cytarabine, daunorubicin, docetaxel, doxifluridine, doxorubicin, epirubicin, etoposide, fluorouracil, gemcitabine, herceptin, idarubicin, mechlorethamine, melphalan, mercaptopurine, methotrexate, mitoxantrone, oxaliplatin, paclitaxel, tafluposide, teniposide, tioguanine, retinoic acid, valrubicin, vinblastine, vincristine, vindesine, vinorelbine, receptor tyrosine kinase inhibitors, and combinations thereof. Additional examples of antitumor or chemotherapeutic agents are known in the art.

Second agent choice is dictated by the standard of care for a given cancer type, cancer progression/stage such as isolated versus metastatic and surgical or morbidity status.

It is further contemplated that other adjunct therapies may be administered, where appropriate. For example, the patient may also be administered an extracellular matrix degrading protein, surgical therapy, chemotherapy, a cytotoxic agent, or radiation therapy where appropriate.

### Dosage

The method may comprise administering a therapeutically effective amount of one or more agents consisting of a aminoglycoside antibiotics to a patient in need thereof. The therapeutically effective amount required for use in therapy varies with the nature of the cancer being treated, and the age/condition of the patient. In general, however, doses employed for adult human treatment typically are in the range of 7.5 mg/kg (Wagner et al., Ann Neurol. 2001 49(6):706-11) to about 15 mg/kg per day The dose may be about 0.1 mg/kg to about 15 mg/kg per day. The desired dose may be conveniently administered in a single dose, or as multiple doses administered at appropriate intervals, for example as two, three, four or more sub-doses per day. Multiple doses may be desired, or required.

### Cancer Types

The invention also provides for methods of treating, inhibiting and preventing tumor growth and cancers such as, e.g. brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, craniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hodgkin's disease and non-Hodgkin's lymphoma), sarcomas (including osteosarcoma, chondrosarcoma and fibrosarcoma) as well as for the treatment of nervous system disorders.

### Subject

In some variations the subject is a mammal (e.g. mice, rat, rabbit, bird, guinea pig, dog or cat). In some variations the subject is a human. Other subjects are contemplated, as set forth in the summary of the invention above.

### Kit

Provided herein is a kit, which may be used for inhibiting or reducing the growth, progression and/or reoccurrence of cancer. The kit may comprise one or more agents consisting of a toxic aggregating peptide, a nucleotide sequence that encodes the toxic aggregating peptide and/or an agent that induces expression of an endogenous nucleotide sequence that encodes the toxic aggregating peptide. The agents may be part of a pharmaceutical composition. The kit may further comprise instructions for using the kit and conducting the administering the said agents or formulation.

### EXAMPLES

The following Examples are merely illustrative and are not intended to limit the scope or content of the invention in any way.

### Example 1: Identification of NEFH Frameshift Variants in CMT2 Families

Whole exome sequencing was performed on three affected individuals belonging to different generations of a British family (UK1) diagnosed with autosomal-dominant CMT2. Exome data was analyzed with a strict filtering approach for segregation of non-synonymous heterozygous variants using the F2 Genomes Management Application (GEM.app) software (Gonzalez et al., Human mutation (2013) 34, 842-846). A heterozygous frameshift variant in NEFH was identified as a top candidate for the disease from a list containing six additional variants (Table 2).

TABLE 2
List of candidate genes from UK1 family's exome data

| seattleseq_gene | MIM | pos | Ref | genotype | protein_notation | polyphen | gerp_score |
|---|---|---|---|---|---|---|---|
| NEFH | 162230 | 29886637 | AAG/AAG | AAG/A | p.Asp1004fs*56 | | |
| DNAH17 | 610063 | 76440872 | G/G | G/A | p.S3781L | 0.679 | 5.12 |
| LYST | 606897 | 235944236 | C/C | C/T | p.E1715K | 0.856 | 5.05 |
| MED20 | 612915 | 41877152 | A/A | A/G | p.V93A | 0.309 | 5.91 |
| PFKFB3 | 605319 | 6258150 | T/T | T/C | p.I101T | 0.995 | 5.11 |
| SPAG5 | 615562 | 26905701 | C/C | C/T | p.G1063S | 1 | 3.49 |
| TRAF4 | 602464 | 27076455 | G/G | G/A | p.G425S | 0.025 | 6.11 |

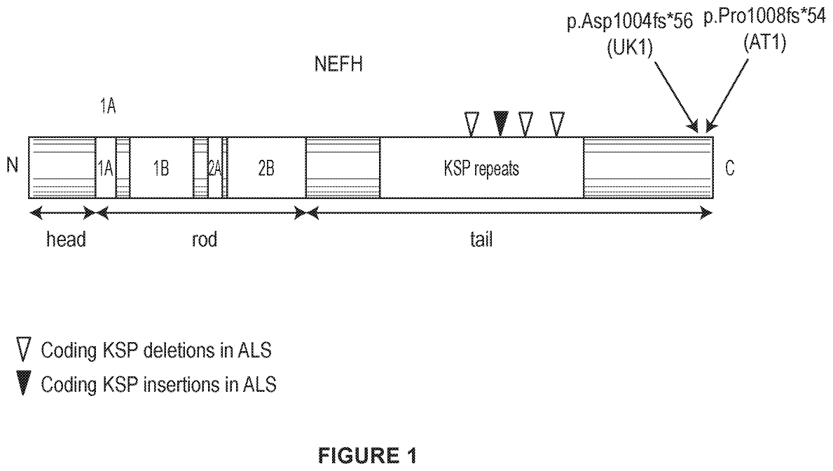

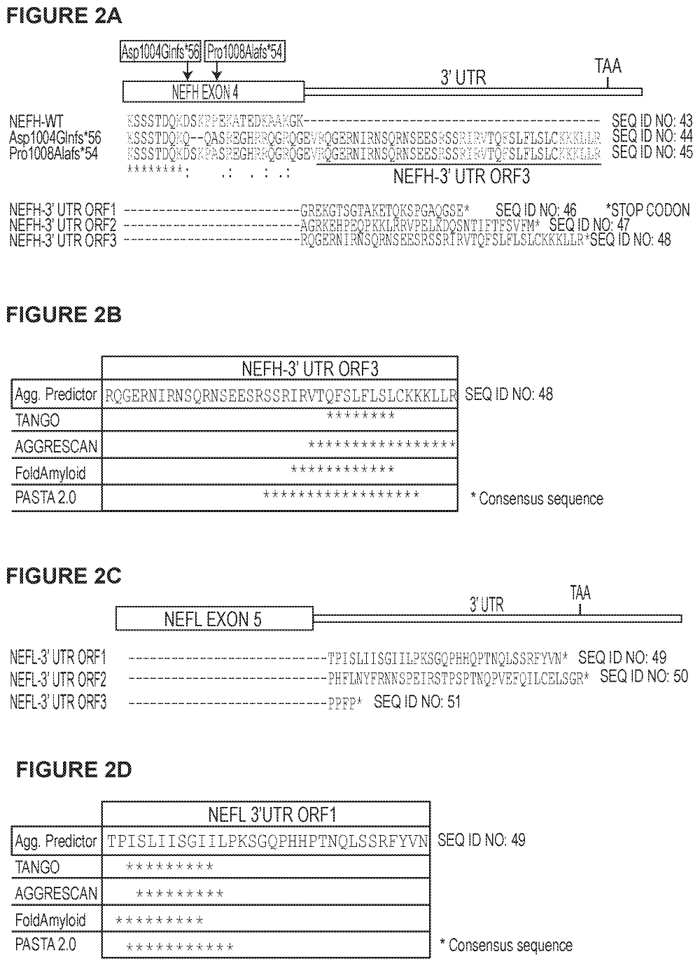

NEFH was selected because NF abnormalities have been previously reported in neurodegenerative diseases, including ALS (Liu et al., CMLS (2004) 61, 3057-3075). In addition, mutations in NEFL, another major NF component, also cause CMT (Jordanova et al., Brain: a journal of neurology (2003) 126, 590-597). The variant co-segregated with the phenotype across three generations in this family. This variant, (c.3010_3011 delGA, p.Asp1004Glnfs*58, chr22:29,886,637) affects the last coding exon and shifts translation into an alternative open reading frame (ORF) resulting in continued translation of an additional 40 amino acids beyond the stop codon in the original ORF (FIG. 2A). The mutant protein retains its major functional domains, including the head, rod and tail domains (FIG. 1). An additional screen 322 CMT families was subsequently performed with whole exome sequencing. This screen identified another CMT2 family (F2) with four affected individuals carrying a nearby heterozygous frameshift variant in NEFH (c.3017_3020 dup, p.Pro1008Alafs*56, chr22:29,886,645). This insertion interestingly also resulted in a stop-loss mutation and translation of the identical ORF as observed in family UK1. Co-segregation of the distinct variants with the phenotype was confirmed by Sanger sequencing in both families Sequencing of DNA from the deceased parent in family F2, who was likely a carrier of the pathogenic variant was not possible. These mutations were not observed in approximately 5,000 additional exomes (Gonzalez et al., Human mutation (2015) 36, 950-956), with a wide range of clinical phenotypes, including other neuropathies, from our own collection and no other frameshift variants in the last exon of NEFH were found.

### Example 2: Identification of Cryptic Amyloidogenic Elements in NEFH and NEFL 3′-UTRs

Because NFs have a considerable tendency to aggregate in neurodegenerative diseases, the intrinsic aggregation propensity of the extension of amino acids present in NEFH in the two CMT2 families was investigated. The web-based aggregation prediction tool TANGO28 was used to analyze aggregation-prone segments based on the physico-chemical principles of beta-sheet formation. According to this algorithm a segment is predicted to aggregate when it contains at least five consecutive residues with a TANGO score above 5% (Fernandez-Escamilla, Nature biotechnology (2004) 22, 1302-1306 incorporated by reference). Analysis of the 3′-UTR extension of amino acids present in the NEFH mutant p.Asp1004Glnfs*58, showed a hot spot for aggregation in eight stretches of amino acids (QFSLFLSL) with a combined score of 250 (FIG. 2A). Aggregation propensity was also analyzed with other prediction tools including AGGRESCAN29, FoldAmyloid30 and PASTA 2.031 that use different algorithms to predict aggregation. All tested tools detected aggregation and an amyloidogenic region in an overlapping stretch of amino acids of the mutant extension (FIG. 2B). This stretch of amino acids predicted to induce aggregation is herein referred to as a cryptic amyloidogenic element (CAE) because it is located in a 3′-UTR and not normally translated in the absence of a genetic mutation or stop codon read-through. Prediction analysis of the other two open reading frames of the NEFH-3′UTR resulted in no aggregation for ORF1 and high aggregation scores for ORF2. These data suggest that any frameshift variants resulting in loss of the NEFH stop codon and translation of cryptic amyloidogenic elements in 3′-UTR would have the potential to cause protein aggregation.

Investigation of the in silico the aggregation propensity of the NEFL 3′-UTR region, revealed that only ORF1 of the 3′-UTR was positive for aggregation prediction with a high TANGO score of 443 comprising the motif ISLIISGII (FIG. 2D). Positive scores for ORF1 were also obtained with AGGRESCAN, FoldAmyloid, and PASTA2.0. In silico results for NEFL suggested that only missense mutations affecting the STOP codon resulting in translation of 3′-UTR ORF1 have the potential to cause aggregation.

### Example 3: Prominent Aggregation in Cultured Neuro-2a Cells Expressing Neurofilaments (NFs) Encoding 3′-UTR CAEs

Following in silico analysis, the potential of the 3′-UTR CAEs identified in affected individuals to cause aggregation in Neuro-2a cells was investigated. Constructs were created encoding GFP-tagged wild type protein (GFP-WT-NEFH) and frameshift mutant protein encoding 40 additional amino acids of ORF3 (GFP-FS-NEFH). GFP-WT-NEFH transfection led to evenly distributed expression in the cytoplasm of Neuro-2a cells. Expression of GFP-FS-NEFH revealed prominent abnormal perinuclear aggregation after 24 hrs post transfection. Quantification showed that over 75% of cells transfected with GFP-FS-NEFH contained aggregates compared to less than 1% in GFP-WT-NEFH cells. Cells transfected with wild-type NEFH retained their typical Neuro-2a cell morphology with ‘axon-like’ projections extending from the cell body. By contrast, GFP-FS-NEFH expressing cells were round-shaped and their axon-like projections were significantly reduced.

In order to experimentally identify which of the additional 40 amino acids are responsible for the aggregation, a series of truncated constructs harboring stop codons (STOP1-STOP4) at different positions throughout the extension of amino acids was created. Cells were transfected with constructs STOP1, STOP2, and STOP3 did not form aggregation in cells and their subcellular distribution pattern was identical to GFP-WT-NEFH. Aggregates were observed in a small proportion of cells (20%) transfected with the longest construct, GFP-STOP4-NEFH; however, aggregation was still more severe in the full-length extension described above (GFP-FS-NEFH). To further characterize the predicted CAE, the most distal 22 amino acids predicted to cause aggregation (SSRIRVTQFSLFLSLCKKKLLR) were cloned directly in frame with the C-terminus end of the GFP-WT-NEF to create the GFP-NEFH-CAE construct. As expected, cells transfected with the GFP-NEFH-CAE construct caused prominent aggregation at the same level observed in cells transfected with the GFP-FS-NEFH. These results prove that the most distal 22 amyloidogenic amino acids are sufficient and necessary for the formation of aggregates.

The ability of the 3′-UTR CAE present in the NEFL to cause aggregation in cells was also investigated. Neuro-2a cells were transfected with constructs encoding GFP-tagged NEFL (GFP-WT-NEFL), NEFL without Stop codon fused in frame with the NEFL-3′-UTR containing the predicted CAE (GFP-NEFL-ORF1), and NEFL fused with the ORF3 3′-UTR, which was not predicted to contain CAE (GFP-NEFL-ORF3). Although a few cells transfected with GFP-WT-NEFL presented aggregates due to the self-assembly nature of NEFL, a large portion of cells adopted NF-like structures, an indication of NF assembly, in about 45% of transfected cells. These filamentous structures can also be explained by the ability of NEFL to self-assemble, in contrast to NEFH, which is an obligate heteropolymer and it requires interaction with either NEFL or NEFM in order to assemble into NFt structures (Evgrafov et al. Nature genetics (2004) 36, 602-606). However, all cells transfected with GFP-NEFL-ORF1 formed prominent aggregation and adopted a rounded shape without forming filamentous structures. In contrast, cells transfected with GFP-NEFL-ORF3 resulted in NFs structures comparable with the GFP-WT-NEFL. The results suggest that specific translation of 3′-UTR CAE present in the first open reading-frame of NEFL is required for the formation of aggregates.

### Example 4: Evaluation of Cells Expressing the NEFH-3′-UTR CAE

As a dramatic difference in cell shape and viability was observed in cells expressing GFP-FS-NEFH compared to wild-type NEFH, the cellular features were quantified. Western blots were performed to show that the levels of protein expression in Neuro-2a cells transfected with GFP-WT-NEFH and GFP-FS-NEFH were similar. Therefore, differences in aggregation cannot be due to protein expression levels. These cells were analyzed using the Incucyte imaging system with time-lapse images taken every 3 hours between 24-48 hrs post transfection. It was observed that the average green (GFP) object area (μm²) per cell was smaller in cells expressing GFP-FS-NEFH (˜120 μm²) compared to GFP-WT-NEFH (>250 μm²), suggesting that GFP-FS-NEFH cells were about 2× smaller. The percentage of confluence of GFP-WT-NEFH positive cells increased with time from 2% to 6.5%, while the confluence of GFP-FS-NEFH remained below 2%. This indicates decreased cell viability after GFP-FS-NEFH transfection. In addition, 48 h post transfection, cells expressing GFP-FS-NEFH started to detach from plates, whereas cells transfected with the GFP-WT-NEFH were still attached and viable several days after transfection. The average green object eccentricity, a parameter that measures object roundness from 0 to 1, where 0 represents a perfect circle, confirmed that GFP-FS-NEFH cells have a more rounded shape. Therefore abnormal aggregation of mutant NEFH may cause a toxic gain-of-function effect leading to loss of neuronal characteristics and loss of cell viability.

To further characterize the NEFH aggregation structures, cells were stained with thioflavin T, a dye commonly used to stain amyloid fibrils with beta-sheet structures in individual tissues (Gonzalez et al., Human mutation (2015) Innovative Genomic Collaboration Using the GENESIS (GEM.app) Platform; Groenning et al., Journal of chemical biology (2010) 3, 1-18). Confocal fluorescent imaging showed strong thioflavin T staining of NEFH aggregates, suggesting a fibrillary amyloid-like type of structure. In order to further understand the composition of the aggregates, transmission electron microscopy was performed in transfected Neuro-2a cells. In cells transiently expressing GFP-FS-NEFH, disordered arrays of filaments of approximately 10 nm in diameter consistent with NF size, were observed. Similar disordered NF inclusion structures were previously reported in the anterior horn cells in an individual with ALS harboring a SOD1 (MIM: 147450) mutation (Kokubo et al., Archives of neurology (1999) 56, 1506-1508). These disordered arrays of filaments were observed in 12/100 images of cells expressing GFP-FS-NEFH in no less than 5 different cells and were never observed in 0/40 GFP-WT-NEFH images or 0/20 images of untransfected cells. Although it was not possible to distinguish untransfected from transfected cells in the EM images, transfection efficiency was ˜70% and close to 20 rounded cells transfected with GFP-FS-NEFH were analyzed, which is a feature of cells containing severe aggregates. Based on the frequency of this feature in cells expressing GFP-FS-NEFH and absence in cells expressing GFP-WT-NEFH or untransfected cells, these structures were likely to be the aggregate.

### Example 5: Expression of NEFH Mutant Protein Disrupts the Neurofilament (NF) Network in Cultured Neuro-2a Cells

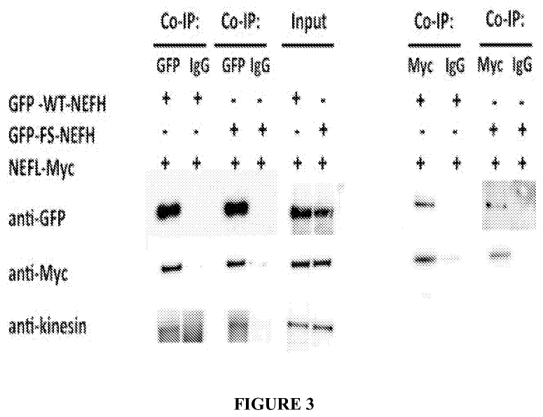

In order to see the effect of the expression of the mutant NEFH on the NF network, GFP-NEFH constructs with a plasmid encoding NEFL fused to a Myc-tag at the C-terminus (NEFL-Myc) were co-transfected. As expected, the GFP-WT-NEFH co-localized with NEFL-Myc protein and assembled into organized NF-like structures. The GFP-FS-NEFL also co-localized with NEFL-Myc, but within the massive aggregates, suggesting arrest and co-aggregation of NEFL and consequently disruption of the NF network. Co-immunoprecipitation experiments were performed to confirm interaction between the mutant NEFH and NEFL. Cell lysates were immunoprecipitated with an anti-GFP antibody to pull-down NEFH. Western blot confirmed that NEFL-Myc was co-immunoprecipitated in cells transfected with either GFP-WT-NEFH or GFP-FS-NEFH (FIG. 3). The reverse co-immunoprecipitation with NEFL-Myc pull-down also confirmed the interaction. Kinesin was further detected in the co-immunoprecipitate in cells transfected with either wild-type or mutant NEFH (FIG. 3).

These results suggest that the mutant NEFH is trapping NEFL, kinesins and possibly other interacting proteins into the aggregates and consequently blocking these proteins from performing their proper functions. Hence, this stop-loss NEFH mutation found in individuals with CMT2 is most likely a toxic gain-of-function mutation. To determine whether the aggregates were affecting exclusively the NF network or additional cytoskeleton components, cells were stained with a tubulin antibody. The microtubule network was normally distributed and assembled in cells transfected with either GFP-WT-NEFH or GFP-FS-NEFH indicating it was not affected by the aggregates.

The NF network has been shown to be important for the spatial subcellular distribution of mitochondria (Wagner et al., The Journal of neuroscience: the official journal of the Society for Neuroscience (2003) 23:9046-58). Therefore cells were stained with an antibody against the mitochondrial outer membrane protein TOM20 (MIM: 601848) to evaluate the effect of the stop-loss mutation. Mitochondria were evenly distributed in cells expressing GFP-WT-NEFH; however, in cells expressing GFP-FS-NEFH, mitochondria accumulated adjacently to the NEFH aggregates. Similarly, it has been shown that NEFL mutations linked to CMT cause altered mitochondrial distribution and co-localization with aggregates Perez-Olle et al., Journal of neurochemistry (2005) 93:861-74. The results support the importance of NF integrity in proper mitochondrial distribution.

### Example 6: Expression of CAE Induces Formation of Amyloid Bodies Upon Physiological Stress

Amyloid bodies are nuclear protein foci that induce a state of cellular dormancy (Audas et al., Developmental Cell (2016) 39:155-168). Amyloid bodies formation is an important cellular mechanism during stress, such as high temperature, hypoxia and acidosis, conditions prevalent in tumor microenvironment. MCF-7, breast cancer cells transfected with GFP-CAE (TOPO GFP vector, Invitrogen) shows cytoplasmic puncta expression under standard growth conditions (37° C., 5% CO₂) compared with a positive control VHL-GFP. When such cells were placed under different environmental stresses, heat shock (43° C.), acidosis and hypoxia (pH 6.0, 1% O₂) (3 hours), GFP-CAE changes from cytoplasmic puncta to co-localization with Congo Red, amyloid marker, positive foci in the nucleus. VHL-GFP was also targeted to amyloid bodies during stress as previously reported. This data supports the hypothesis that CAE aggregates can force growing cells to enter a state of dormancy.

### Example 7: Genome-Wide Analysis of Human 3′-UTR CAE

In order to identify potential candidate genes for causing aggregation through expression of a cryptic amyloidogenic element in the 3′-UTR, bioinformatics aggregation prediction analysis of all human 3′-UTR sequences was performed. Human 3′-UTR sequences were acquired from the UTRef section of UTRdb, a curated collection of eukaryotic 5′ and 3′-UTRs. The UTRef section contains 34,619 3′-UTR sequences from genes retrieved from the National Center for Biotechnology Information (NCBI) RefSeq transcripts (Grillo et al., Nucleic acids research (2010) 38, D75-80. These sequences were translated into the three forward reading-frames to simulate stop-loss mutations caused by either missense (frame 1) or frameshift (frames 2 and 3) mutations. After filtering out amino acid sequences with over 90% similarity and genes of uncertain function (LOC symbols), approximately 12,400 genes per reading-frame were annotated with the aggregation prediction programs, TANGO and PASTA.

Next, sequences were filtered for highly stringent threshold aggregation scores, above 200 for TANGO and below −4 for PASTA. These score cutoffs were based on the aggregation prediction scores obtained for the NEFH-3′-UTR. Sequences that lack an alternative stop codon were filtered-out since they would likely be degraded by the non-stop decay mechanism. It has been reasoned that the stability of stop-loss mRNAs and/or proteins decreases as the distance between the mutated stop codon and the next alternative stop codon increases (Hamby et al., Human genomics (2011) 5, 241-264). Therefore, only sequences containing an alternative stop codon within 50 amino acids were considered.

After these filter criteria were applied, we obtained 4,861 genes, approximately 1,600 genes per reading-frame, containing a 3′-UTR sequence that has a high potential for aggregation if translated. Table 1 contains a list of the putative CAE's identified, MIM number corresponding to the Online Mendelian Inheritance in Man (OMIM®) catalog of human genes, TANGO and PASTA score for protein aggregation, University of California, Santa Cruz (UCSC) genome browser transcript ID and human genome version 19 (Hg19) chromosome location reference number for use on Genome Reference Consortium (GRC), NCBI website. The frame 1, 2 or 3 are also indicated in Table 1 for each of these genomic loci.

Although our results suggest that a large number of genes have the potential to cause aggregation as the result of a stop-loss mutation, several physiological factors that vary in different intracellular micro environments influence aggregation, such as, temperature, pH, pressure and protein concentration (Huang et al., Archives of biochemistry and biophysics 568, 46-55). Moreover, the frequency of stop-loss caused by a missense mutation within the stop codon (frame 1) is very low, 0.027% (609 of 2,207,918 variants) as observed in the NHLBI GO Exome Sequencing Project. Finally, in order to cause significant protein aggregation disease, the protein must be expressed in cells that can be negatively impacted by aggregations such as postmitotic neurons, and protein expression levels must overwhelm the cell's ability to clear aggregations.

Next, disease-associated genes previously reported to aggregate that contain a predicted 3′-UTR-CAE in any frame were identified. A list with the top 21 high-risk aggregation genes was compiled as shown in Table 3.

TABLE 3
List of aggregation disease genes predicted to contain a 3′-UTR CAE.

| | Gene | | | | | | | | |
|---|---|---|---|---|---|---|---|---|---|
| | MM | | Phenotype | Frame 1 | | Frame 2 | | Frame 3 | |
| Gene | Number | Disease | MM | TANGO | PASTA | TANGO | PASTA | TANGO | PASTA |
| APOA2 | 107670 | Apolipoprotein A-II | 143890 | 182.8 | No | 0.0 | No | 0.0 | No |
| | | deficiency | | | | | | | |
| ATXN2 | 601517 | Spinocerebellar ataxia | 183090 | 989.1 | −7.9 | 76.5 | No | 853.0 | −7.7 |
| | | 2 | | | | | | | |
| B2M | 109700 | Amyloidosis | 105200 | No | No | 2265.7 | −17.6 | 181.1 | No |
| FGB | 134830 | Hypofibrinogenemia, | 202400 | 358.3 | −3.5 | 998.2 | −13.9 | | No |
| | | congenital | | | | | | | |
| FUS | 137070 | Amyotrophic lateral | 608030 | 1323.0 | No | No | no | 48.4 | No |
| | | sclerosis (ALS) | | | | | | | |
| IAPP | 147940 | Type II diabetes | 125853 | No | No | 541.1 | −10.9 | | No |
| LYZ | 153450 | Amyloidosis | 105200 | 183.1 | No | 962.9 | −6.5 | 160.7 | −5.7 |
| NOTCH3 | 600276 | Cerebral arteriopathy | 125310 | No | No | No | No | 566.0 | −5.1 |
| PRNP | 176640 | Prion disease | 606688 | 0.0 | No | 896.1 | −10.3 | | No |
| RHO | 180380 | Retinitis pigmentoas | 613731 | 697.1 | −7.0 | No | No | | No |
| SAA1 | 104750 | Amyloidosis | 105210 | No | No | No | No | 326.2 | −4.1 |
| SNCA | 163890 | Parkinson's disease | 168601 | No | No | 4.4 | No | 1442.5 | −9.0 |
| SOD1 | 147450 | Amyotrophic lateral | 105400 | No | No | 568.1 | −7.0 | 6.7 | No |
| | | sclerosis (ALS) | | | | | | | |
| TARDBP | 605078 | Amyotrophic lateral | 612069 | 714.6 | −5.8 | No | No | 464.4 | −10.0 |
| | | sclerosis (ALS) | | | | | | | |
| NEFL | 162280 | Charcot- | 607734 | 445.6 | −8.7 | No | No | 5.5 | −2.9 |
| | | marie tooth | | | | | | | |
| BSCL2 | 606158 | Neuropathy, distal | 600794 | 126.7 | No | 0.6 | No | 1.2 | No |
| | | hereditary motor | | | | | | | |
| MAPT | 157140 | Parkinson disease | 168600 | No | no | 156.6 | No | 6.7 | No |
| TTR | 176300 | Amyloidosis | 105210 | 0.0 | no | 0.0 | No | 112.3 | No |

The aggregation of 5 genes (NEFH, NEFL, FUS, TDP43 and SOD1) from the list of aggregation disease genes containing 3′UTR CAEs was tested. Aggregation was confirmed in three of those genes (NEFH, NEFL and FUS) in transfected cells. Neuro-2a cells transfected with FUS fused in frame with its predicted 3′-UTR-CAE frame 1 (GFP-FUS-CAE) showed prominent aggregation in the cytoplasm and neuronal projections of transfected cells. In comparison, wild-type FUS (GFP-WT-FUS) localized to the nucleus. Cells transfected with SOD1 and TARDBP fused with their respective 3′-UTR-CAE did not result in protein aggregation. These results showed that aggregation induced by translation of the 3-UTR CAE is not a NF exclusive phenomenon; however, it is important to validate the bioinformatics aggregation prediction of the 3′-UTR-CAE since other intrinsic protein factors may interfere with protein structure.

### Example 8: Time Lapse Images Shows Toxic Effect of Aggregates in Neuro-2a Cells

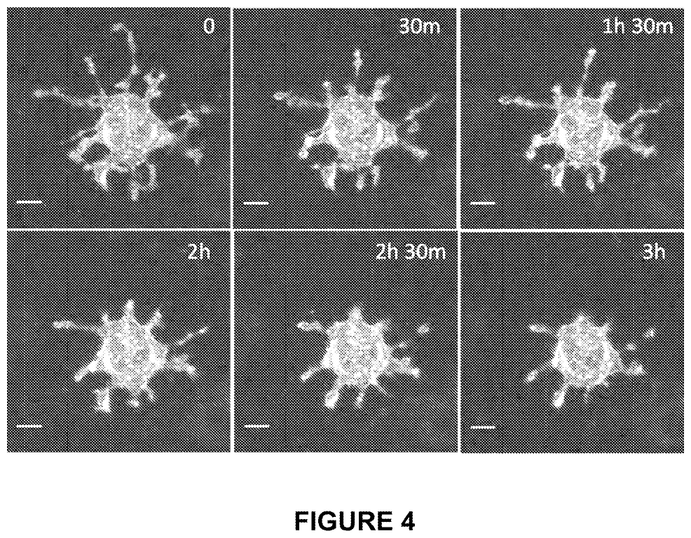

Time-lapse of Z-stacked images of Neuro-2a cells expressing the NEFH-FS-CAE aggregates as described in Example 7, depicted progressive loss of neuronal projections before cell rounding and detachment (FIG. 4). The abnormal aggregation of mutant NEFH might cause a toxic gain-of-function effect leading to loss of neuronal characteristics and loss of cell viability. It was concluded that cells become ‘dormant’ as described by (Audas et al., Developmental Cell (2016) 39:155-168). However, as our cells detach, they do not remain viable for a long time.

### Example 9: Analysis of 3′-UTR-CAE of Human Genes that Undergo Functional Translational Read-Through (FTR)

Translation of mRNA usually terminates at the first in-frame stop codon, however, translational read-through can occur, when translation bypasses the first termination codon and continues until the next stop codon in the 3′-UTR, resulting in protein extension. This phenomenon is commonly observed in less complex organisms, but very rare in mammals Although prone to errors, translation termination error rate is below 0.1% in humans. However, some termination suppressor drugs such as aminoglycosides can significantly increase this error. Interestingly, a few mammalian genes have been shown to undergo a programmed translational read-through mechanism termed functional translational read-through (FTR) (Schueren and Thorns, PLoS Genet (2016) 12(8):e1006196). This mechanism allows the generation of distinct protein isoforms that can modulate function of the regular-sized protein. Because evolution would be expected to suppress translation of toxic cryptic amyloidogenic elements in FTR genes, the aggregation propensity of the 3-UTR of these genes was evaluated. Tango was calculated for 13 genes with confirmed FTR and as expected none of those genes have a positive CAE at frame 1. Fisher's exact test demonstrated that the number of FTR genes with absent CAE was statistically significant (p=0.003865).

### Example 10: Determining the Ability of Amyloidogenic Aggregates to Inhibit Cancer Cell Growth In Vitro

Cancer cells heavily depend on the autophagic and proteasomal pathways to scavenge nutrients that are essential for their growth and survival. Therefore in hypoxic tumor cells, impaired clearance of the protein aggregates will result in apoptotic cell death.

The ability of toxic amyloidogenic peptide aggregates to inhibit the growth and survival of in vitro models of cancer is investigated. Amyloidogenic peptide aggregates are exogenously-expressed in neuroblastoma cells and cell morphology growth and survival is evaluated under both hypoxic and normal culture conditions.

Neuroblastoma cancer cells (Neuro-2a cells) are maintained in DMEM (supplemented with 10% fetal bovine serum and 1% antibiotic/antimycotic agent) at 37° C. in 5% CO₂ and 21% O₂. For hypoxia experiments, the cells are incubated in a hypoxia chamber (5% CO₂ and 1% O₂) at 37° C. Cells are transfected with mammalian expression vectors encoding GFP-tagged wild type protein (GFP-WT-NEFH) or NEFL without a stop codon fused in frame with the NEFL-3′-UTR containing the predicted CAE, toxic protein aggregate construct (GFP-NEFL-ORF1, cDNA cDNA encoding for the most distal 22 amino acids predicted to cause aggregation, SSRIRVTQFSLFLSLCKKKLLR). Cells will subjected to hypoxic conditions or maintained in normal culture conditions. Following 24 h post transfection, cell morphology is analyzed by fluorescence microscopy. The percentage of cells displaying aggregation is quantified. These cells are then analyzed using the Incucyte imaging system with time-lapse images taken every 3 h between 24-48 h post transfection. Average green (GFP) object area (μm²) per cell and average green object eccentricity, a parameter that measures object roundness from 0 to 1, where 0 represents a perfect circle, is measured. Cell viability is determined by measuring apoptosis (Caspase activity), autophagy (autophagy markers), membrane integrity (permeability assay), metabolic activity (MTT assay) and cell proliferation (BrdU staining), cell monolayer confluence and detachment from the culture dish. In addition, protein expression levels are determined by Western blotting.

### Example 11: Expression of Human 3′-UTR CAE in Cancer Cell Lines

Glioblastoma cells are affected by toxicity mediated by CAE expression. Lentivirus was produced in order to obtain stable and inducible expression of cytotoxic amyloidogenic protein in glioblastoma. The mutant NEFH-CAE (as described in example 5 and as shown in FIG. 2) was cloned in the pLVX-TRE3G vector (Clonetech) under the control of a TRE (tetracycline response element) inducible promoter that expresses high levels of protein, but only in the presence of doxycycline (Dox) and Tet-On 3G transactivator protein (pLVX-Tet3G). When bound to Dox, the constitutively expressed Tet-on 3G protein undergoes a conformational change that allows it to bind to the TRE sequence and activate transcription. The GFP-TRE3G vector expressing only GFP was also generated to be used as an experimental control. Preliminary results in glioblastoma cells co-transfected with NEFH-CAE-TRE3G and pLVX-Tet3G in the presence and absence of Dox showed tight regulation of the gene and formation of aggregates after 48 hr exposure to Dox. No leakage of expression was observed in the absence of Dox.

These preclinical data demonstrate the effectiveness of an inducible CAE under the control of a response element in models of cancer whereby the amyloidogenic protein was able to induce controllable cytotoxicity in cancer cells. The results also demonstrate the feasibility of using an inducible promoter, which is contemplated as an aspect of the invention.

### Example 12: Generation of CRISPR Guide RNAs to Induce the Activation of CAE in Oncogenes Highly Expressed in Tumors

Oncogenes and tumor associated genes that contain a predicted 3′UTR-CAE in any frame were filtered and selected for study. Those genes were retrieved from the Tumor Associated Gene (TAG) database. A list with 105 candidate genes with high aggregation prediction scores was compiled as shown in Table 4.

Those genes are potential good targets to activate toxic aggregates in specific types of cancer cells. A list of genes highly expressed in tumors in different types of cancer (based on immunohistochemistry) was collected using the Human Protein Atlas (www.proteinatlas.org/). CRISPR guide RNAs (gRNAs) were designed to genes targets using Deskgen (https://www.deskgen.com/) to target the terminal end of the last exons to activate the expression of the CAE in different type of cancer including glioblastoma, breast cancer, melanoma, pancreatic cancer and liver cancer as shown in Table 5. Guide RNAs were designed to have the PAM site immediately upstream (less than 20 nucleotide) or right at the stop codon. Guide RNAs were designed for the following genes: ABL2, BCL9, BIRC5, CCNE1, CEP55, CSF1R, FUS, IRF2, MYB, NUMA1, SALL4, SEMA3E, SERTAD2, SPHK1, TACC3, TIAM1 (Table 5). The same approach is suitable for design of guide RNAs for other genes with CAE's described herein.

To summarize, Table 5 provides the following information: 1) the oncogenes and tumor associated genes containing CAE and that are highly expressed in particular tumors; 2) OMIM reference sequence; 3) the CAE reading frame; 4) NCBI reference sequence; 5) the cancer type in which the gene is highly expressed; 6) the associated ‘guide RNA’ DNA target sequence; 7) the protospacer adjacent motif sequence (PAM); 8) the target exon number in the gene of interest.

### Example 13: Expression of Human 3′-UTR NEFH-CAE in Vertebrate Zebrafish

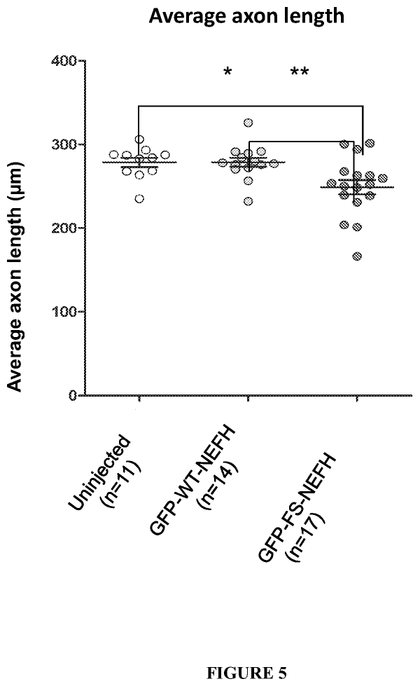

In order to assess the effects of the NEFH-CAE variant in vivo, RNA was injected into one-cell stage zebrafish embryos. Equal amounts of RNA encoding either GFP-WT-NEFH or GFP-NEFH-CAE were injected into transgenic Tg(Olig2:DsRed) (Kucenas et al., Neuron Glia Biol (2008) 4:71-81) embryos at a dosage at which there was no apparent effect on body morphology, but a measurable difference in motor neuron outgrowth. The common path of the caudal anterior primary motor neurons at 48 hours post fertilization (hpf) was assessed. The GFP-NEFH-CAE RNA injected embryos were found to have significantly decreased axon lengths compared to both GFP-WT-NEFH and uninjected larvae (FIG. 5), while there is no significant difference between the motor neurons lengths of the uninjected embryos and embryos injected with GFP-WT-NEFH RNA. These data indicate that the addition of the 3′-UTR CAE can function through a toxic mechanism. Such agents containing a nucleotide sequence that encodes the amyloidogenic peptide could be used to inhibit growth of neoplastic (e.g. cancer) cells in mammals by inducing cytotoxicity through exogenous expression of the amyloidogenic peptide.

### Example 14: Determining the Ability of Amyloidogenic Aggregates to Inhibit Cancer Cell Growth In Vivo

Prolonged use of aminoglycoside antibiotics, such as gentamicin could potentially induce aggregation of NEFL. Therefore the effect of aminoglycoside antibiotics on growth rate of Neuro-2a cells are investigated using a xenograft model.

C57/BL6 mice (6-8 week old) are purchased from Charles River Laboratory and used according to an approved protocol. Mice (n=8 mice per group) are injected with 1×10⁶ Neuro-2a cancer cells into the appropriate matching tissue. Once tumors reached 150 mm² (30 days), mice are randomized and divided by multiple case and control groups and each group received the treatment. The treatment with control agent and aminoglycoside antibiotic is performed daily for 7 days. The aminoglycoside antibiotics and vehicle control are delivered via tail vein injection. Control agent is injected at FDA recommended dosage per body weight. The aminoglycoside antibiotics are injected at FDA approved dosages per body weight. Tumor sizes are subsequently measured using a caliper. Animals are sacrificed once the tumor reached 2000 mm³ in size. Protein aggregation can be detected by immunostaining and/or by blue native polyacrylamide gel electrophoresis to detect high molecular weight aggregates complexes.

### Example 15: Determining the Ability of NEFL Stop Codon Mutation to Induce NEFL Aggregation and Inhibit Cancer Cell Growth In Vitro

The effect of CRISPR/Cas9-mediated mutation of the Stop codon NEFL gene, on the growth rate of Neuro-2a cells is investigated. Cells are infected with Adv-CRISPR/Cas9 and single guide RNA and growth rate and survival is measured in vitro.

Neuroblastoma cancer cells (Neuro-2a cells) are maintained in as previously described. For hypoxia experiments, the cells are incubated in a hypoxia chamber (5% CO₂ and 1% O₂) at 37° C. Neuro-2a cells are seeded at a density of 8.0×10⁴ cells per well in 24-well plates. The following day, the cells are transduced with AdV.Cas9 (150 TCID₅₀/cell), AdV.gRNA for the stop codon of NEFL (targeted to 50 TCID₅₀/cell) and AdV.Δ2.donor (10 TU/cell). To serve as negative controls, cells are either mock-transduced or are transduced with AdV.Cas9 (150 TCID₅₀/cell) and AdV.Δ2. donor (10 TU/cell). Following 24 h post transduction, cell morphology is analyzed by fluorescence microscopy. The percentage of cells displaying aggregation is quantified. These cells are then analyzed using the IncuCyte live-cell imaging system (Essen Instruments) with time-lapse images taken every 3 h between 24-48 h post transduction. Average green (GFP) object area (μm²) per cell and average green object eccentricity, a parameter that measures object roundness from 0 to 1, where 0 represents a perfect circle, is measured using the IncuCyte Zoom software. A total of 36 images are acquired per well for each time point. Cell viability is determined by measuring apoptosis (Caspase activity), autophagy (autophagy markers), membrane integrity (permeability assay), metabolic activity (MTT assay) and cell proliferation (BrdU staining), cell monolayer confluence and detachment from the culture dish. In addition, protein expression levels are determined by Western blotting.

To confirm genetic modification, surveyor assay and sequencing analysis is performed on lysates of cells infected with plasmid DNA as described above. After infection, the cells are incubated at 37° C. for 72 h before genomic DNA extraction. Genomic DNA is extracted using the QuickExtract DNA extraction kit (Epicentre) following the manufacturer's protocol. Briefly, cells are resuspended in QuickExtract solution and incubated at 65° C. for 15 minutes and 98° C. for 10 minutes. Extracted genomic DNA is immediately processed or stored at −20° C.

The genomic region surrounding a CRISPR target site for each gene is PCR amplified, and products are purified using QiaQuick Spin Column (Qiagen) following manufacturer's protocol. A total of 400 ng of the purified PCR products are mixed with 2 μl 10×Taq polymerase PCR buffer (Enzymatics) and ultrapure water to a final volume of 20 μl, and subjected to a re-annealing process to enable heteroduplex formation: 95° C. for 10 min, 95° C. to 85° C. ramping at −2° C./s, 85° C. to 25° C. at −0.25° C./s, and 25° C. hold for 1 minute. Following re-annealing, products are treated with Surveyor nuclease and Surveyor enhancer S (Transgenomics) following the manufacturer's recommended protocol, and analyzed on 4-20% Novex TBE poly-acrylamide gels (Life Technologies). Gels are stained with SYBR Gold DNA stain (Life Technologies) for 30 minutes and imaged with a Gel Doc gel imaging system (Bio-rad). Quantification is based on relative band intensities, as a measure of the fraction of cleaved DNA.

### Example 16: Determining the Ability of NEFL Stop Codon Mutation to Induce NEFL Aggregation and Inhibit Cancer Cell Growth In Vivo

Glioblastoma Multiforme (GBM) is recognized as one of the most deadly cancers characterized by cellular atypia, severe necrosis, and high rate of angiogenesis (Chen et al., Biochim Biophys Acta. (2013) 1836(1):158-65). The U87 glioma mouse xenograft is a commonly used model of GBM.

Swiss nude mice (4-6 weeks old) are purchased from Charles River Laboratory and used according to an approved protocol. Mice (n=10 mice per group) are subjected to intracranial implantation of human 1×10⁵ U87 cells (suspended in 10 μL of sterile PBS) into the right frontal hemisphere using a stereotactic fixation device. Implants were placed 2 mm from the midline, 3 mm anterior to the bregma, and 3 mm deep. Cells were slowly injected over 120-180 sec.

Once tumors develop, mice are treated with CRISPR constructs, AdV.Cas9 (150 TCID₅₀/cell), AdV.gRNA for the stop codon of NEFL (targeted to 50 TCID₅₀/cell) and AdV.Δ2.donor (10 TU/cell) via transcranial injection. To serve as negative controls, mice are injected with vector controls without guide RNA. Tumor sizes are subsequently measured using a caliper. Following completion of the study, animals are sacrificed and tumor cells are analyzed. Protein aggregation can be detected by immunostaining and/or by blue native polyacrylamide gel electrophoresis to detect high molecular weight aggregates complexes. Cell viability is determined by measuring apoptosis (Caspase activity), autophagy (autophagy markers), and membrane integrity (permeability assay). In addition, protein expression levels are determined by Western blotting. To confirm genetic modification, surveyor assay and sequencing analysis is performed on extracted tumor lysates as described above.

TABLE 1

| | | Frame 1 | | | | Frame 2 | | |
|---|---|---|---|---|---|---|---|---|
| | MIM | | | UCSC | | | | UCSC |
| Gene | Number | TANGO | PASTA | Transcript ID | Hg19 Location | TANGO | PASTA | Transcript ID |
| A2M | 103950 | NA | NA | NA | NA | 444.572 | −4.840821 | uc001qvk.1 |
| AACS | 614364 | NA | NA | NA | NA | 479.236 | −8.254097 | uc001uhc.3 |
| AADACL3 | NA | NA | NA | NA | NA | NA | NA | NA |
| AAED1 | NA | 808.469 | −11.059608 | uc004awm.3 | chr9: 99403533- | NA | NA | NA |
| | | | | | 99404040 | | | |
| AAK1 | 616405 | NA | NA | NA | NA | 348.205 | −16.518709 | uc010fdm.1 |
| AARS | 601065 | NA | NA | NA | NA | NA | NA | NA |
| AARSD1 | 613212 | NA | NA | NA | NA | 461.255 | −5.153679 | uc002icd.3 |
| AASDH | 614365 | 713.979 | −13.239747 | uc010ihb.3 | chr4: 57204457- | NA | NA | NA |
| | | | | | 57204567 | | | |
| AASS | 605113 | 582.035 | −7.439998 | uc003vka.3 | chr7: 121713598- | 637.449 | −7.620471 | uc003vka.3 |
| | | | | | 121716542 | | | |
| ABCA1 | 600046 | NA | NA | NA | NA | 388.284 | −4.521932 | uc004bcl.3 |
| ABCA10 | 612508 | NA | NA | NA | NA | 328.634 | −6.503525 | uc010wqs.1 |
| ABCA13 | 607807 | 238.481 | −6.088288 | uc003top.2 | chr7: 48267027- | NA | NA | NA |
| | | | | | 48269729 | | | |
| ABCA3 | 601615 | 1103.92 | −1.072616 | uc002cpy.1 | chr16: 2325879- | 433.694 | −7.71141 | uc002cpy.1 |
| | | | | | 2326674 | | | |
| ABCA5 | 612503 | 1698.08 | −13.121301 | uc002jib.2 | chr17: 67240576- | 1263.96 | −5.626877 | uc002jib.2 |
| | | | | | 67243677 | | | |
| ABCA6 | 612504 | 786.504 | −12.259205 | uc002jhw.1 | chr17: 67074847- | 258.66 | −6.915899 | uc002jhy.3 |
| | | | | | 67075113 | | | |
| ABCA8 | 612505 | NA | NA | NA | NA | 1835.46 | −4.341029 | uc002jhp.3 |
| ABCA9 | 612507 | 271.367 | −7.774992 | uc010dez.3 | chr17: 66970773- | 320.413 | −2.497322 | uc010dez.3 |
| | | | | | 66972055 | | | |
| ABCB11 | 603201 | 449.027 | −5.647562 | uc010zda.1 | chr2: 169779449- | NA | NA | NA |
| | | | | | 169780131 | | | |
| ABGB4 | 171060 | 253.859 | −3.877531 | NA | NA | 240.091 | −7.230367 | uc003uiy.3 |
| ABGB5 | 611785 | 735.569 | −13.100493 | uc003suv.4 | chr7: 20698713- | NA | NA | NA |
| | | | | | 20700017 | | | |
| ABCB6 | 605452 | NA | NA | NA | NA | NA | NA | NA |
| ABCC2 | 601107 | NA | NA | NA | NA | 753.088 | −8.857978 | uc001kqf.2 |
| ABCC3 | 604323 | 1177.17 | −3.596524 | uc002isl.3 | chr17: 48768562- | NA | NA | NA |
| | | | | | 48769063 | | | |
| ABCC4 | 605250 | 972.818 | −14.03781 | uc010afj.3 | chr13: 95672083- | NA | NA | NA |
| | | | | | 95673828 | | | |
| ABCC5 | 605251 | 239.31 | −6.379406 | uc003fmh.3 | chr3: 183701541- | NA | NA | NA |
| | | | | | 183702682 | | | |
| ABCG2 | 603756 | NA | NA | NA | NA | NA | NA | NA |
| ABHD12B | NA | NA | NA | NA | NA | NA | NA | NA |
| ABHD13 | NA | NA | NA | NA | NA | NA | NA | NA |
| ABHD16A | 142620 | NA | NA | NA | NA | NA | NA | NA |
| ABHD17B | NA | NA | NA | NA | NA | NA | NA | NA |
| ABHD3 | 612197 | NA | NA | NA | NA | NA | NA | NA |
| ABI1 | 603050 | NA | NA | NA | NA | 504.03 | −5.957448 | uc010qdg.2 |
| ABI3BP | 606279 | 611.542 | −5.030571 | uc003duj.3 | chr3: 100468179- | NA | NA | NA |
| | | | | | 100469338 | | | |
| ABL2 | 164690 | 239.103 | −3.707258 | uc001gmk.3 | chr1: 179085983- | 222.186 | −6.350043 | uc009wxe.3 |
| | | | | | 179086398 | | | |
| ABLIM3 | 611305 | NA | NA | NA | NA | 214.821 | −9.927634 | uc003lpy.2 |
| ABRACL | NA | 1303.28 | −12.261925 | uc003qil.2 | chr6: 139364041- | 1251.97 | −10.879057 | uc003qil.2 |
| | | | | | 139364439 | | | |
| ABT1 | NA | 367.166 | −5.924138 | uc003nii.3 | chr6: 26598874- | 821.258 | −5.181718 | uc003nii.3 |
| | | | | | 26600277 | | | |
| ABTB1 | 608308 | 330.242 | −4.901486 | uc003ejr.3 | chr3: 127399319- | NA | NA | NA |
| | | | | | 127399769 | | | |
| ACAD10 | 611181 | 479.2 | −4.621561 | uc001tsq.3 | chr12: 112194278- | 543.092 | −3.435728 | uc009zvw.2 |
| | | | | | 112194911 | | | |
| ACAD8 | 604773 | 1540.84 | −2.777604 | uc009zdc.3 | chr11: 134129078- | 230.064 | −5.678102 | uc010sco.1 |
| | | | | | 134129819 | | | |
| ACAD9 | 611103 | NA | NA | NA | NA | 698.927 | −2.283534 | uc003ela.4 |
| ACAT1 | 607809 | 456.646 | −6.245965 | uc001pjw.1 | chr11: 108006851- | 1051.57 | −8.685169 | uc001pjw.1 |
| | | | | | 108007666 | | | |
| ACBD3 | 606809 | NA | NA | NA | NA | NA | NA | NA |
| ACBD5 | 616618 | NA | NA | NA | NA | NA | NA | NA |
| ACCS | 608405 | 372.662 | −4.538341 | uc010rfm.1 | chr11: 44098926- | NA | NA | NA |
| | | | | | 44101170 | | | |
| ACD | 609377 | NA | NA | NA | NA | NA | NA | NA |
| ACE2 | 300335 | NA | NA | NA | NA | 748.984 | −12.960602 | uc004cxa.1 |
| ACER2 | 613492 | NA | NA | NA | NA | NA | NA | NA |
| ACER3 | NA | 365.258 | −4.733146 | uc010rsg.1 | chr11: 76726166- | NA | NA | NA |
| | | | | | 76726305 | | | |
| ACHE | 100740 | NA | NA | NA | NA | 710.831 | −6.070792 | uc003uxd.3 |
| ACKR3 | 610376 | NA | NA | NA | NA | NA | NA | NA |
| ACKR4 | 606065 | NA | NA | NA | NA | NA | NA | NA |
| ACMSD | 608889 | NA | NA | NA | NA | NA | NA | NA |
| ACO1 | 100880 | 1183.77 | −3.950379 | uc010mjh.1 | chr9: 32436141- | 1779.39 | 0.910319 | uc010mjh.1 |
| | | | | | 32436395 | | | |
| ACOT1 | 614313 | 393.8 | −7.12892 | uc001xol.1 | chr14: 74010360- | NA | NA | NA |
| | | | | | 74010498 | | | |
| ACOT8 | 608123 | NA | NA | NA | NA | NA | NA | NA |
| ACOT9 | 300862 | NA | NA | NA | NA | NA | NA | NA |
| ACOX2 | 601641 | 623.549 | −13.118453 | uc003dkl.3 | chr3: 58490863- | NA | NA | NA |
| | | | | | 58490950 | | | |
| ACP5 | 171640 | NA | NA | NA | NA | NA | NA | NA |
| ACRC | 300369 | 313.529 | −4.831006 | uc004eae.2 | chrX: 70832833- | NA | NA | NA |
| | | | | | 70833433 | | | |
| ACRV1 | 102525 | 978.292 | −11.899704 | uc001qcl.3 | chr11: 125542229- | NA | NA | NA |
| | | | | | 125542487 | | | |
| ACSL3 | 602371 | NA | NA | NA | NA | NA | NA | NA |
| ACSL4 | 300157 | 801.242 | −12.145498 | uc004eoi.2 | chrX: 108884564- | NA | NA | NA |
| | | | | | 108887257 | | | |
| ACSL6 | 604443 | 540.579 | −8.068454 | uc003kvw.2 | chr5: 131285667- | NA | NA | NA |
| | | | | | 131289926 | | | |
| ACSM3 | 145505 | NA | NA | NA | NA | 297.312 | −9.431658 | uc002dhr.3 |
| ACSM6 | NA | 1011.55 | −5.277201 | uc001kke.3 | chr10: 96988542- | NA | NA | NA |
| | | | | | 96988686 | | | |
| ACSS1 | 614355 | NA | NA | NA | NA | 332.032 | −4.319356 | uc002wua.3 |
| ACTA1 | 102610 | NA | NA | NA | NA | 1308.73 | −10.342939 | uc001htm.3 |
| ACTA2 | 102620 | 498.901 | −4.578403 | uc001kfp.3 | chr10: 90694831- | NA | NA | NA |
| | | | | | 90694979 | | | |
| ACTBL2 | 614835 | 526.359 | −4.241581 | uc003jrm.3 | chr5: 56775843- | NA | NA | NA |
| | | | | | 56777403 | | | |
| ACTC1 | 102540 | 1394.05 | −9.933252 | uc001ziu.1 | chr15: 35080297- | NA | NA | NA |
| | | | | | 35082612 | | | |
| ACTG1 | 102560 | NA | NA | NA | NA | NA | NA | NA |
| ACTL6A | 604958 | NA | NA | NA | NA | 574.602 | −8.03775 | uc003fjw.3 |
| ACTL8 | NA | 406.261 | −5.477243 | uc001bat.3 | chr1: 18153015- | NA | NA | NA |
| | | | | | 18153558 | | | |
| ACTN2 | 102573 | NA | NA | NA | NA | NA | NA | NA |
| ACTR1B | 605144 | 321.382 | −5.220611 | uc002syb.2 | chr2: 98272402- | NA | NA | NA |
| | | | | | 98273320 | | | |
| ACTR2 | 604221 | 761.27 | −5.313251 | uc010yqf.1 | chr2: 65492379- | NA | NA | NA |
| | | | | | 65492501 | | | |
| ACTR3C | NA | 804.909 | −8.280315 | uc003wgu.2 | chr7: 149944301- | 1009.4 | −8.482162 | uc003wgu.2 |
| | | | | | 149944658 | | | |
| ACTRT1 | 300487 | 237.643 | −4.652226 | uc004eum.3 | chrX: 127184941- | NA | NA | NA |
| | | | | | 127185054 | | | |
| ACTRT3 | 608534 | NA | NA | NA | NA | 1221.28 | −6.955979 | uc003ffs.2 |
| ACVR1B | 601300 | 1171.64 | −14.646106 | uc001rzm.3 | chr12: 52385817- | NA | NA | NA |
| | | | | | 52386125 | | | |
| ACVR1C | 608981 | NA | NA | NA | NA | 616.644 | −6.203043 | uc010foe.3 |
| ACY1 | 104620 | NA | NA | NA | NA | 957.566 | −9.619426 | uc011beb.2 |
| ACYP1 | 600875 | 896.449 | −14.901447 | uc001xrg.3 | chr14: 75519928- | NA | NA | NA |
| | | | | | 75520146 | | | |
| ADA | 608958 | NA | NA | NA | NA | NA | NA | NA |
| ADAM10 | 602192 | NA | NA | NA | NA | NA | NA | NA |
| ADAM18 | NA | NA | NA | NA | NA | NA | NA | NA |
| ADAM21 | 603713 | 363.035 | −10.490319 | uc001xmd.3 | chr14: 70926386- | NA | NA | NA |
| | | | | | 70926622 | | | |
| ADAM33 | 607114 | NA | NA | NA | NA | 518.425 | −8.100465 | uc010zqg.1 |
| ADAM9 | 602713 | NA | NA | NA | NA | 670.938 | −6.530036 | uc003xmp.3 |
| ADAMTS12 | 606184 | 1471.22 | −9.572451 | uc003jib.1 | chr5: 33751303- | NA | NA | NA |
| | | | | | 33751443 | | | |
| ADAMTS14 | 607506 | 315.533 | −8.232168 | uc001jrg.3 | chr10: 72520610- | NA | NA | NA |
| | | | | | 72522195 | | | |
| ADAMTS16 | 607510 | NA | NA | NA | NA | 264.978 | −5.048283 | uc003jdl.3 |
| ADAMTS19 | 607513 | NA | NA | NA | NA | NA | NA | NA |
| ADAMTS2 | 604539 | NA | NA | NA | NA | NA | NA | NA |
| ADAMTS20 | 611681 | 1475.01 | −13.481115 | uc001rno.1 | chr12: 43816622- | 1995.58 | −13.328557 | uc001rno.1 |
| | | | | | 43817496 | | | |
| ADAMTS5 | 605007 | 496.474 | −9.440545 | uc002ymg.3 | chr21: 28290231- | NA | NA | NA |
| | | | | | 28296371 | | | |
| ADAMTS6 | 605008 | NA | NA | NA | NA | NA | NA | NA |
| ADAMTS9 | 605421 | 1001.21 | −14.96224 | uc011bfp.1 | chr3: 64507157- | 602.119 | −1.164763 | uc011bfp.1 |
| | | | | | 64507344 | | | |
| ADAMTSL1 | 609198 | NA | NA | NA | NA | NA | NA | NA |
| ADAMTSL4 | 610113 | NA | NA | NA | NA | NA | NA | NA |
| ADAT1 | 604230 | NA | NA | NA | NA | 386.125 | −4.423724 | uc002feo.2 |
| ADCY10 | 605205 | NA | NA | NA | NA | 685.055 | −10.012084 | uc001ger.3 |
| ADCY2 | 103071 | 207.824 | −5.703427 | uc003jdz.1 | chr5: 7826985- | NA | NA | NA |
| | | | | | 7830194 | | | |
| ADCY8 | 103070 | 1120.56 | −15.695436 | uc003ytd.4 | chr8: 131792547- | 1391.32 | −14.878036 | uc003ytd.4 |
| | | | | | 131792635 | | | |
| ADCY9 | 603302 | 691.119 | −8.202348 | uc002cvx.3 | chr16: 4012650- | NA | NA | NA |
| | | | | | 4015775 | | | |
| ADGB | 614630 | NA | NA | NA | NA | 1050.46 | −9.009208 | uc003qlp.3 |
| ADGRE3 | 606101 | NA | NA | NA | NA | 316.395 | −8.14128 | uc002mzi.4 |
| ADGRF2 | NA | NA | NA | NA | NA | 421.326 | −5.633664 | uc003oyy.3 |
| ADGRG3 | NA | 685.432 | −6.680478 | uc010cdc.3 | chr16: 57712265- | NA | NA | NA |
| | | | | | 57712751 | | | |
| ADGRG5 | NA | NA | NA | NA | NA | NA | NA | NA |
| ADGRL3 | 616417 | NA | NA | NA | NA | NA | NA | NA |
| ADGRL4 | 616419 | NA | NA | NA | NA | 564.397 | −5.908999 | uc001diq.4 |
| ADH1A | 103700 | 647.721 | −0.931022 | uc003hur.2 | chr4: 100197523- | 984.541 | −7.456647 | uc003hur.2 |
| | | | | | 100197780 | | | |
| ADH1B | 103720 | NA | NA | NA | NA | 575.393 | −5.678532 | uc003hus.4 |
| ADH4 | 103740 | NA | NA | NA | NA | 603.553 | −7.227677 | uc011ced.2 |
| ADH5 | 103710 | 452.092 | −9.977706 | uc003huk.1 | chr4: 99996376- | NA | NA | NA |
| | | | | | 99997415 | | | |
| ADH6 | 103735 | NA | NA | NA | NA | NA | NA | NA |
| ADH7 | 600086 | 710.698 | −9.368407 | uc021xqj.1 | chr4: 100333418- | NA | NA | NA |
| | | | | | 100334304 | | | |
| ADI1 | 613400 | 470.128 | −4.791654 | uc002qxp.4 | chr2: 3501690- | NA | NA | NA |
| | | | | | 3502733 | | | |
| ADK | 102750 | NA | NA | NA | NA | NA | NA | NA |
| ADORA2B | 600446 | 494.701 | −7.626874 | uc002gpd.1 | chr17: 15878657- | NA | NA | NA |
| | | | | | 15879210 | | | |
| ADORA3 | 600445 | NA | NA | NA | NA | NA | NA | NA |
| ADPGK | 611861 | NA | NA | NA | NA | NA | NA | NA |
| ADPRH | 603081 | 790.356 | −8.775883 | uc003ecs.3 | chr3: 119306726- | NA | NA | NA |
| | | | | | 119308792 | | | |
| ADPRM | NA | NA | NA | NA | NA | 497.11 | −6.759758 | uc002gmt.3 |
| ADRA1A | 104221 | NA | NA | NA | NA | 294.121 | −5.226475 | uc003xfc.1 |
| ADRB2 | 109690 | NA | NA | NA | NA | 426.898 | −5.842956 | uc003lpr.2 |
| ADRBK1 | 109635 | NA | NA | NA | NA | NA | NA | NA |
| ADTRP | 614343 | 1047.92 | −11.977491 | uc003nab.3 | chr6: 11713888- | 623.671 | −4.003574 | uc003nab.3 |
| | | | | | 11714710 | | | |
| AEBP2 | NA | 551.538 | −5.447398 | uc001ree.2 | chr12: 19671052- | NA | NA | NA |
| | | | | | 19675173 | | | |
| AEN | 610177 | 454.286 | −4.809185 | uc010bnl.2 | chr15: 89170047- | 1308.82 | −8.506475 | uc010bnl.2 |
| | | | | | 89170572 | | | |
| AFAP1L2 | 612420 | NA | NA | NA | NA | NA | NA | NA |
| AFF1 | 159557 | NA | NA | NA | NA | NA | NA | NA |
| AFF2 | 300806 | NA | NA | NA | NA | 630.194 | −8.348309 | uc004fco.3 |
| AFG3L2 | 604581 | 740.898 | −7.02342 | uc002kqz.2 | chr18: 12328943- | 509.45 | −4.270726 | uc002kqz.2 |
| | | | | | 12329563 | | | |
| AFMID | NA | NA | NA | NA | NA | NA | NA | NA |
| AFP | 104150 | 376.027 | −7.547135 | uc003hgz.1 | chr4: 74321348- | NA | NA | NA |
| | | | | | 74321492 | | | |
| AGA | 613228 | NA | NA | NA | NA | NA | NA | NA |
| AGAP6 | NA | 907.015 | −6.949954 | uc001jix.4 | chr10: 51769947- | NA | NA | NA |
| | | | | | 51770259 | | | |
| AGAP9 | NA | NA | NA | NA | NA | 571.029 | −7.747569 | uc009xnl.2 |
| AGBL2 | NA | NA | NA | NA | NA | NA | NA | NA |
| AGFG1 | 600862 | NA | NA | NA | NA | NA | NA | NA |
| AGGF1 | 608464 | 952.013 | −5.243169 | uc003ket.3 | chr5: 76359078- | 498.817 | −7.301877 | uc003kes.3 |
| | | | | | 76361058 | | | |
| AGL | 610860 | NA | NA | NA | NA | 307.473 | −5.162082 | uc001dsi.1 |
| AGO2 | 606229 | 424.967 | −5.654323 | uc003yvn.3 | chr8: 141541264- | NA | NA | NA |
| | | | | | 141542142 | | | |
| AGPAT1 | 603099 | NA | NA | NA | NA | NA | NA | NA |
| AGPAT5 | 614796 | NA | NA | NA | NA | 228.58 | −5.151283 | uc003wqo.3 |
| AGPS | 603051 | NA | NA | NA | NA | NA | NA | NA |
| AGR2 | 606358 | NA | NA | NA | NA | 380.214 | −8.477037 | uc003str.3 |
| AGRN | 103320 | NA | NA | NA | NA | 258.61 | −3.774371 | uc001ack.2 |
| AGTRAP | 608729 | NA | NA | NA | NA | 594.833 | −6.857241 | uc001asu.3 |
| AHCTF1 | 610853 | 463.989 | −4.613284 | uc001ibv.2 | chr1: 247002402- | NA | NA | NA |
| | | | | | 247004107 | | | |
| AHR | 600253 | NA | NA | NA | NA | NA | NA | NA |
| AHSA2 | NA | 290.348 | −6.742453 | uc002sbb.2 | chr2: 61413922- | NA | NA | NA |
| | | | | | 61414686 | | | |
| AHSG | 138680 | NA | NA | NA | NA | 239.356 | −4.281995 | uc003fqj.3 |
| AIFM2 | 605159 | 599.288 | −4.511345 | uc010qjg.2 | chr10: 71857979- | NA | NA | NA |
| | | | | | 71859375 | | | |
| AIM1L | NA | 546.035 | −6.090322 | uc001bmd.4 | chr1: 26648350- | NA | NA | NA |
| | | | | | 26648458 | | | |
| AIMP1 | 603605 | NA | NA | NA | NA | 1457.35 | −6.347748 | uc011cfg.2 |
| AK1 | 103000 | NA | NA | NA | NA | NA | NA | NA |
| AK4 | 103030 | NA | NA | NA | NA | 956.855 | −16.875932 | uc001dby.3 |
| AK9 | 615358 | 1674.55 | −15.107617 | uc003ptr.4 | chr6: 109952900- | 1242.49 | −13.557257 | uc003ptr.4 |
| | | | | | 109954113 | | | |
| AKAP1 | 602449 | 1100.25 | −7.144316 | uc002iux.3 | chr17: 55197686- | NA | NA | NA |
| | | | | | 55198710 | | | |
| AKAP11 | 604696 | NA | NA | NA | NA | NA | NA | NA |
| AKAP12 | 604698 | NA | NA | NA | NA | NA | NA | NA |
| AKAP14 | 300462 | 547.65 | −4.880603 | uc004esd.3 | chrX: 119037559- | NA | NA | NA |
| | | | | | 119037620 | | | |
| AKAP5 | 604688 | 880.743 | −6.412715 | uc001xhd.4 | chr14: 64936397- | NA | NA | NA |
| | | | | | 64941221 | | | |
| AKAP6 | 604691 | 1022.74 | −3.77998 | uc010aml.3 | chr14: 33201888- | 1266.11 | −5.444838 | uc010aml.3 |
| | | | | | 33203819 | | | |
| AKAP9 | 604001 | 441.827 | −7.533427 | uc003uld.4 | chr7: 91625130- | 240.029 | −5.532238 | uc003ule.2 |
| | | | | | 91625366 | | | |
| AKR1B10 | 604707 | NA | NA | NA | NA | NA | NA | NA |
| AKR1B15 | 616336 | NA | NA | NA | NA | 383.94 | −4.37522 | uc011kpr.2 |
| AKR1C3 | 603966 | 587.591 | −8.577187 | uc001ihr.3 | chr10: 5149696- | NA | NA | NA |
| | | | | | 5149878 | | | |
| AKR7A2 | 603418 | NA | NA | NA | NA | 339.705 | −5.292473 | uc001bbw.3 |
| AKR7A3 | 608477 | NA | NA | NA | NA | 319.082 | −8.796156 | uc001bbv.1 |
| AKT3 | 611223 | NA | NA | NA | NA | 1108.45 | −10.380657 | uc001iab.2 |
| ALB | 103600 | NA | NA | NA | NA | NA | NA | NA |
| ALDH1A1 | 100640 | NA | NA | NA | NA | 842.026 | −13.754001 | uc011lsg.2 |
| ALDH3A2 | 609523 | 270.655 | −7.292776 | uc002gwa.1 | chr17: 19578871- | NA | NA | NA |
| | | | | | 19580908 | | | |
| ALDH6A1 | 603178 | NA | NA | NA | NA | NA | NA | NA |
| ALDOC | 103870 | 539.976 | −6.457156 | uc002hbp.3 | chr17: 26900133- | NA | NA | NA |
| | | | | | 26900543 | | | |
| ALG13 | 300776 | 611.856 | −5.920844 | uc004epi.2 | chrX: 110931230- | 333.719 | −14.075961 | uc022ccn.1 |
| | | | | | 110933623 | | | |
| ALG14 | 612866 | 526.33 | −7.360435 | uc001dra.2 | chr1: 95448279- | NA | NA | NA |
| | | | | | 95448631 | | | |
| ALG2 | 607905 | NA | NA | NA | NA | 808.078 | −11.828651 | uc004azf.3 |
| ALG5 | 604565 | NA | NA | NA | NA | 925.397 | −8.563312 | uc001uvy.3 |
| ALG9 | 606941 | NA | NA | NA | NA | 919.403 | −8.179035 | uc001ply.3 |
| ALKBH5 | 613303 | 296.645 | −5.224656 | uc010cpw.3 | chr17: 18111711- | NA | NA | NA |
| | | | | | 18113267 | | | |
| ALLC | NA | NA | NA | NA | NA | NA | NA | NA |
| ALMS1P | NA | NA | NA | NA | NA | 902.32 | −4.079886 | uc010yrl.2 |
| ALOX5AP | 603700 | NA | NA | NA | NA | 803.367 | −8.95589 | uc010tdr.2 |
| ALS2 | 606352 | 1362.19 | −13.030073 | uc002uyp.4 | chr2: 202586713- | 308.953 | −4.803593 | uc002uyo.3 |
| | | | | | 202587753 | | | |
| ALS2CR12 | NA | NA | NA | NA | NA | NA | NA | NA |
| ALX1 | 601527 | 762.036 | −6.751349 | uc001tae.4 | chr12: 85695254- | 661.801 | −12.364502 | uc001tae.4 |
| | | | | | 85695561 | | | |
| AMACR | 604489 | 438.163 | −4.032429 | uc003jik.2 | chr5: 33997112- | 585.045 | −6.349009 | uc003jig.3 |
| | | | | | 33997355 | | | |
| AMBN | 601259 | NA | NA | NA | NA | 487.762 | −6.729336 | uc003hfl.3 |
| AMBP | 176870 | NA | NA | NA | NA | NA | NA | NA |
| AMELY | 410000 | NA | NA | NA | NA | 283.558 | −5.212522 | uc004fqz.3 |
| AMICA1 | NA | 586.95 | −7.011785 | uc001psg.2 | chr11: 118064442- | NA | NA | NA |
| | | | | | 118065039 | | | |
| AMMECR1 | 300195 | NA | NA | NA | NA | 524.486 | −5.394369 | uc004eoo.3 |
| AMMECR1L | NA | NA | NA | NA | NA | 466.589 | −5.071615 | uc002tpl.3 |
| AMN1 | NA | NA | NA | NA | NA | 1466.05 | −16.466447 | uc001rko.4 |
| AMOT1 | 300410 | NA | NA | NA | NA | 385.85 | −6.464716 | uc004epr.3 |
| AMPD2 | 102771 | NA | NA | NA | NA | NA | NA | NA |
| AMPD3 | 102772 | 818.811 | −7.752449 | uc010rbz.1 | chr11: 10527432- | NA | NA | NA |
| | | | | | 10529126 | | | |
| AMTN | 610912 | NA | NA | NA | NA | NA | NA | NA |
| ANG | 105850 | NA | NA | NA | NA | 316.136 | −3.864586 | uc001vxw.4 |
| ANGEL2 | NA | NA | NA | NA | NA | NA | NA | NA |
| ANGPT1 | 601667 | NA | NA | NA | NA | 821.848 | −9.055921 | uc011lhv.2 |
| ANGPT2 | 601922 | NA | NA | NA | NA | 344.727 | −9.271782 | uc003wqj.4 |
| ANHX | NA | 687.656 | −7.978357 | uc010tci.2 | chr12: 133794898- | NA | NA | NA |
| | | | | | 133795470 | | | |
| ANKH | 605145 | NA | NA | NA | NA | 1077.43 | −11.35707 | uc003jfm.4 |
| ANKHD1 | 610500 | NA | NA | NA | NA | 397.842 | −4.649907 | uc003lfr.3 |
| ANKMY1 | NA | 710.728 | −8.062947 | uc002vzf.3 | chr2: 241492166- | NA | NA | NA |
| | | | | | 241492228 | | | |
| ANKRA2 | 605787 | 767.844 | −11.615776 | uc003kcu.2 | chr5: 72848025- | NA | NA | NA |
| | | | | | 72848621 | | | |
| ANKRD11 | 611192 | NA | NA | NA | NA | NA | NA | NA |
| ANKRD13A | 615123 | NA | NA | NA | NA | 1114.61 | −12.884117 | uc009zvm.2 |
| ANKRD17 | 615929 | 393.775 | −7.71959 | uc011cbd.1 | chr4: 73978801- | 624.054 | −6.545105 | uc003hgo.3 |
| | | | | | 73978986 | | | |
| ANKRD20A5P | NA | 340.647 | −5.409111 | uc010xag.2 | chr18: 14184108- | 583.575 | −4.056875 | uc010xag.2 |
| | | | | | 14185386 | | | |
| ANKRD26 | 610855 | 1433.5 | −13.245119 | uc001itg.2 | chr10: 27293045- | NA | NA | NA |
| | | | | | 27294518 | | | |
| ANKRD28 | 611122 | 218.829 | −6.560457 | uc011avy.1 | chr3: 15708744- | NA | NA | NA |
| | | | | | 15711776 | | | |
| ANKRD31 | NA | 2051.18 | −18.17751 | uc003kdo.2 | chr5: 74364122- | NA | NA | NA |
| | | | | | 74364343 | | | |
| ANKRD34C | NA | NA | NA | NA | NA | NA | NA | NA |
| ANKRD40 | NA | NA | NA | NA | NA | NA | NA | NA |
| ANKRD42 | NA | 898.042 | −9.833575 | uc009yvi.2 | chr11: 82923706- | NA | NA | NA |
| | | | | | 82926242 | | | |
| ANKRD44 | NA | 1079.61 | −5.257466 | uc002utz.4 | chr2: 197831741- | NA | NA | NA |
| | | | | | 197832162 | | | |
| ANKRD45 | NA | NA | NA | NA | NA | 303.662 | −4.732646 | uc001gja.1 |
| ANKRD49 | NA | NA | NA | NA | NA | 772.87 | −6.450876 | uc001pew.3 |
| ANKRD50 | NA | NA | NA | NA | NA | 252.236 | −5.06163 | uc010inw.3 |
| ANKRD6 | 610583 | NA | NA | NA | NA | NA | NA | NA |
| ANKRD7 | 610731 | NA | NA | NA | NA | 249.167 | −4.448399 | uc003vji.3 |
| ANKS4B | 609901 | 334.94 | −6.973966 | uc010bwp.1 | chr16: 21262142- | NA | NA | NA |
| | | | | | 21263750 | | | |
| ANO1 | 610108 | 608.89 | −1.475041 | uc001opj.3 | chr11: 70034111- | 1057.62 | −9.425129 | uc001opj.3 |
| | | | | | 70035652 | | | |
| ANO4 | 610111 | 634.713 | −6.013856 | uc001thw.2 | chr12: 101521639- | NA | NA | NA |
| | | | | | 101522419 | | | |
| ANO5 | 608662 | NA | NA | NA | NA | 1215.31 | −8.672186 | uc001mqi.2 |
| ANO6 | 608663 | NA | NA | NA | NA | NA | NA | NA |
| ANO7 | 605096 | NA | NA | NA | NA | 342.41 | −5.296089 | uc002wax.2 |
| ANP32B | NA | NA | NA | NA | NA | 415.007 | −8.032003 | uc004aya.3 |
| ANXA1 | 151690 | NA | NA | NA | NA | NA | NA | NA |
| ANXA3 | 106490 | 635.139 | −3.923796 | uc003hld.3 | chr4: 79531270- | NA | NA | NA |
| | | | | | 79531605 | | | |
| ANXA5 | 131230 | NA | NA | NA | NA | 206.891 | −6.255208 | uc003idu.4 |
| ANXA7 | 186360 | NA | NA | NA | NA | 632.426 | −5.04571 | uc001jtz.2 |
| AOC2 | 602268 | NA | NA | NA | NA | 873.793 | −12.432538 | uc002ibt.3 |
| AOX1 | 602841 | NA | NA | NA | NA | NA | NA | NA |
| AP1G1 | 603533 | 494.064 | −5.973242 | uc002faz.3 | chr16: 71762905- | NA | NA | NA |
| | | | | | 71766960 | | | |
| AP2A2 | 607242 | NA | NA | NA | NA | 844.592 | −7.370147 | uc001lss.3 |
| AP3M1 | 610366 | 451.442 | −7.290908 | uc001jwf.3 | chr10: 75880015- | NA | NA | NA |
| | | | | | 75883567 | | | |
| AP4E1 | 607244 | 1069.1 | −9.153814 | NA | NA | NA | NA | NA |
| AP4S1 | 607243 | NA | NA | NA | NA | NA | NA | NA |
| AP5M1 | 614368 | NA | NA | NA | NA | 1641.13 | −6.642618 | uc001xcu.4 |
| AP5S1 | 614824 | 354.576 | −6.011422 | uc002wjt.3 | chr20: 3804945- | NA | NA | NA |
| | | | | | 3805580 | | | |
| APAF1 | 602233 | NA | NA | NA | NA | 647.448 | −8.408864 | uc001tgc.3 |
| APBA1 | 602414 | NA | NA | NA | NA | 975.827 | −12.902824 | uc004ahh.2 |
| APBA2 | 602712 | NA | NA | NA | NA | 626.836 | −5.244 | uc001zcm.1 |
| APBA3 | 604262 | NA | NA | NA | NA | 493.96 | −6.972095 | uc002lyo.1 |
| APBB1IP | 609036 | 271.513 | −4.507734 | uc001isr.3 | chr10: 26790107- | NA | NA | NA |
| | | | | | 26790867 | | | |
| APEH | 102645 | 685.725 | −9.056655 | uc003cxf.3 | chr3: 49720776- | NA | NA | NA |
| | | | | | 49720934 | | | |
| API5 | 609774 | 864.582 | −12.477097 | uc001mxf.2 | chr11: 43364006- | NA | NA | NA |
| | | | | | 43366080 | | | |
| APIP | 612491 | NA | NA | NA | NA | 348.586 | −5.135784 | uc010reo.1 |
| APLP2 | 104776 | NA | NA | NA | NA | NA | NA | NA |
| APOA5 | 606368 | NA | NA | NA | NA | 585.826 | −4.418834 | uc001ppr.3 |
| APOB | 107730 | 318.78 | −4.098363 | uc002red.3 | chr2: 21224301- | 396.406 | −5.268298 | uc002red.3 |
| | | | | | 21224601 | | | |
| APOBEC3C | 607750 | 466.341 | −5.283477 | uc003awr.3 | chr22: 39414393- | NA | NA | NA |
| | | | | | 39414825 | | | |
| APOE | 107741 | NA | NA | NA | NA | NA | NA | NA |
| APOH | 138700 | NA | NA | NA | NA | 408.75 | −7.843125 | uc002jfn.4 |
| APOL2 | 607252 | 758.563 | −7.263197 | uc011amm.2 | chr22: 36622255- | NA | NA | NA |
| | | | | | 36623449 | | | |
| APPL1 | 604299 | 707.545 | −5.288662 | uc010hnb.3 | chr3: 57301866- | NA | NA | NA |
| | | | | | 57302049 | | | |
| AQP10 | 606578 | 719.654 | −7.410811 | uc001feu.3 | chr1: 154296957- | NA | NA | NA |
| | | | | | 154297801 | | | |
| AQP9 | 602914 | 703.744 | −5.331671 | uc010ugx.1 | chr15: 58476335- | 693.546 | −7.83981 | uc010ugx.1 |
| | | | | | 58478110 | | | |
| AQR | 610548 | NA | NA | NA | NA | 443.142 | −5.602549 | uc001ziv.3 |
| ARAP2 | 606645 | NA | NA | NA | NA | NA | NA | NA |
| ARF1 | 103180 | NA | NA | NA | NA | NA | NA | NA |
| ARF6 | 600464 | NA | NA | NA | NA | 658.523 | −9.832598 | uc001wxg.4 |
| ARFGAP1 | 608377 | NA | NA | NA | NA | NA | NA | NA |
| ARFGAP2 | 606908 | NA | NA | NA | NA | NA | NA | NA |
| ARFGEF1 | 604141 | 573.884 | −8.644808 | uc003xxm.2 | chr8: 68109884- | 782.151 | −0.72839 | uc003xxm.2 |
| | | | | | 68111168 | | | |
| ARFIP1 | 605928 | NA | NA | NA | NA | 339.665 | −4.340257 | uc003imz.3 |
| ARFIP2 | 601638 | 1049.74 | −4.586033 | uc001mdk.3 | chr11: 6495913- | NA | NA | NA |
| | | | | | 6498342 | | | |
| ARG1 | 608313 | NA | NA | NA | NA | NA | NA | NA |
| ARGFX | 611164 | NA | NA | NA | NA | NA | NA | NA |
| ARHGAP10 | 609746 | 471.426 | −9.236217 | uc003ile.1 | chr4: 148778812- | NA | NA | NA |
| | | | | | 148779639 | | | |
| ARHGAP11A | 610589 | 590.501 | −14.721695 | uc010ubw.1 | chr15: 32930047- | 545.869 | −4.135009 | uc001zgw.3 |
| | | | | | 32931868 | | | |
| ARHGAP18 | 613351 | NA | NA | NA | NA | 1068.66 | −9.253961 | uc003qbr.3 |
| ARHGAP21 | 609870 | 623.056 | −6.47277 | uc001isd.1 | chr10: 24917943- | NA | NA | NA |
| | | | | | 24918846 | | | |
| ARHGAP29 | 610496 | 1328.32 | −13.234418 | uc001dqj.4 | chr1: 94634463- | 1481.78 | −2.102763 | uc001dqj.4 |
| | | | | | 94639424 | | | |
| ARHGAP32 | 608541 | 485.072 | −7.188398 | uc009zco.3 | chr11: 128834955- | NA | NA | NA |
| | | | | | 128838801 | | | |
| ARHGAP33 | 614902 | 596.063 | −5.597539 | uc002obs.2 | chr19: 36279332- | 540.237 | −5.334934 | uc002obs.2 |
| | | | | | 36279724 | | | |
| ARHGAP42 | 615936 | 600.289 | −7.300864 | uc001pge.2 | chr11: 100859533- | NA | NA | NA |
| | | | | | 100861656 | | | |
| ARHGAP6 | 300118 | 497.673 | −5.601986 | uc004cum.1 | chrX: 11155663- | NA | NA | NA |
| | | | | | 11156982 | | | |
| ARHGEF12 | 604763 | NA | NA | NA | NA | NA | NA | NA |
| ARHGEF15 | 608504 | NA | NA | NA | NA | 588.111 | −9.013614 | uc002glb.2 |
| ARHGEF16 | NA | NA | NA | NA | NA | 572.903 | −5.814258 | uc009vli.1 |
| ARHGEF28 | 612790 | NA | NA | NA | NA | 991.164 | −20.859578 | uc010izf.3 |
| ARHGEF3 | 612115 | NA | NA | NA | NA | NA | NA | NA |
| ARHGEF35 | NA | NA | NA | NA | NA | 551.466 | −4.698665 | uc003wdz.2 |
| ARHGEF38 | NA | NA | NA | NA | NA | 803.666 | −5.778281 | uc003hxu.3 |
| ARHGEF4 | 605216 | NA | NA | NA | NA | NA | NA | NA |
| ARID1B | 614556 | NA | NA | NA | NA | NA | NA | NA |
| ARID2 | 609539 | 748.131 | −3.647276 | uc001ros.1 | chr12: 46298862- | 458.524 | −4.323699 | uc001rot.1 |
| | | | | | 46301819 | | | |
| ARID5A | 611583 | NA | NA | NA | NA | NA | NA | NA |
| ARL13B | 608922 | NA | NA | NA | NA | NA | NA | NA |
| ARL14EPL | NA | NA | NA | NA | NA | 450.012 | −6.208547 | uc021yct.1 |
| ARL15 | NA | NA | NA | NA | NA | NA | NA | NA |
| ARL17A | NA | NA | NA | NA | NA | 377.564 | −5.267015 | uc002iki.4 |
| ARL4A | 604786 | NA | NA | NA | NA | 1192.97 | −5.089979 | uc003ssp.3 |
| ARL5B | 608909 | 346.451 | −4.37522 | uc001iqd.1 | chr10: 18964146- | NA | NA | NA |
| | | | | | 18966940 | | | |
| ARL6IP1 | 607669 | NA | NA | NA | NA | 349.582 | −6.040094 | uc010van.1 |
| ARL6IP6 | 616495 | NA | NA | NA | NA | 513.919 | −7.641352 | uc002tyn.3 |
| ARL8B | 616596 | NA | NA | NA | NA | 626.989 | −4.117285 | uc003bqg.3 |
| ARMCX5 | NA | NA | NA | NA | NA | 1393.15 | −13.368919 | uc004ejg.3 |
| ARNTL2 | 602550 | 374.488 | −6.281078 | uc001mko.3 | chr11: 13408304- | NA | NA | NA |
| | | | | | 13408812 | | | |
| ARNTL2 | 614517 | 1444.54 | −11.222628 | uc001rht.2 | chr12: 27573466- | NA | NA | NA |
| | | | | | 27578746 | | | |
| ARPC3 | 604225 | 1085.21 | −8.487821 | uc001tqq.3 | chr12: 110872706- | NA | NA | NA |
| | | | | | 110872959 | | | |
| ARRDC4 | NA | 532.714 | −7.347509 | uc002bui.4 | chr15: 98514418- | 1778.2 | −10.378009 | uc002bui.4 |
| | | | | | 98517068 | | | |
| ARRDC5 | NA | 260.005 | −4.845527 | uc002mbm.3 | chr19: 4890449- | NA | NA | NA |
| | | | | | 4891057 | | | |
| ARSA | 607574 | 212.333 | −4.682161 | uc003bna.4 | chr22: 51061182- | NA | NA | NA |
| | | | | | 51063572 | | | |
| ARSB | 611542 | 1430.63 | −11.448138 | uc003kfq.3 | chr5: 78073037- | NA | NA | NA |
| | | | | | 78076219 | | | |
| ARSE | 300180 | NA | NA | NA | NA | NA | NA | NA |
| ART4 | 110600 | NA | NA | NA | NA | 660.984 | −4.837777 | uc001rcl.1 |
| ARV1 | 611647 | NA | NA | NA | NA | 417.429 | −6.384341 | uc009xfl.1 |
| ASAP1 | 605953 | NA | NA | NA | NA | 399.088 | −6.623233 | uc003ysz.2 |
| ASB1 | 605758 | 782.918 | −6.278528 | uc002vyg.3 | chr2: 239355153- | NA | NA | NA |
| | | | | | 239360891 | | | |
| ASB11 | 300626 | NA | NA | NA | NA | NA | NA | NA |
| ASB16 | 615056 | NA | NA | NA | NA | 777.825 | −4.660612 | uc002ifl.1 |
| ASB17 | NA | NA | NA | NA | NA | NA | NA | NA |
| ASB7 | 615052 | 643.587 | −2.207764 | uc002bwj.3 | chr15: 101170256- | 582.774 | −12.640694 | uc002bwj.3 |
| | | | | | 101170414 | | | |
| ASMTL | 300162 | 209.512 | −5.012961 | uc004cpx.2 | chrX: 1522032- | NA | NA | NA |
| | | | | | 1522161 | | | |
| ASNSD1 | NA | 581.213 | −9.207436 | uc002uqt.3 | chr2: 190535453- | NA | NA | NA |
| | | | | | 190535557 | | | |
| ASPA | 608034 | 704.701 | −9.561641 | uc010ckg.3 | chr17: 3402383- | NA | NA | NA |
| | | | | | 3402700 | | | |
| ASPDH | NA | NA | NA | NA | NA | 215.226 | −4.7723 | uc010enz.3 |
| ASPH | 600582 | NA | NA | NA | NA | 496.231 | −5.797451 | uc011leg.2 |
| ASPM | 605481 | 579.048 | −7.543463 | uc001gtw.4 | chr1: 197057248- | NA | NA | NA |
| | | | | | 197057334 | | | |
| ASS1 | 603470 | NA | NA | NA | NA | NA | NA | NA |
| ASTE1 | NA | 1105.42 | −9.631582 | uc010htm.1 | chr3: 130732721- | NA | NA | NA |
| | | | | | 130732900 | | | |
| ASTN1 | 600904 | NA | NA | NA | NA | 776.727 | −5.953168 | uc001glb.1 |
| ASTN2 | 612856 | NA | NA | NA | NA | 457.558 | −4.805341 | uc004bjp.2 |
| ASXL1 | 612990 | 678.249 | −9.511004 | uc002wxt.3 | chr20: 30959973- | NA | NA | NA |
| | | | | | 30960352 | | | |
| ASZ1 | 605797 | NA | NA | NA | NA | NA | NA | NA |
| ATAD2 | 611941 | NA | NA | NA | NA | 1233.45 | −16.054851 | uc011lii.2 |
| ATAD5 | 609534 | 849.499 | −17.384089 | uc002hfs.1 | chr17: 29221932- | NA | NA | NA |
| | | | | | 29222295 | | | |
| ATE1 | 607103 | NA | NA | NA | NA | NA | NA | NA |
| ATF1 | 123803 | 361.076 | −8.81154 | uc001rww.4 | chr12: 51213563- | NA | NA | NA |
| | | | | | 51214943 | | | |
| ATF3 | 603148 | NA | NA | NA | NA | NA | NA | NA |
| ATF7IP | 613644 | 1227.63 | −9.06265 | uc001rbu.3 | chr12: 14628959- | 1772.4 | −3.498198 | uc001rbu.3 |
| | | | | | 14629347 | | | |
| ATF7IP2 | 613645 | 1770.3 | −21.277169 | uc002czu.3 | chr16: 10576107- | 1745.23 | −0.334273 | uc002c2u.3 |
| | | | | | 10577495 | | | |
| ATG101 | 615089 | NA | NA | NA | NA | 1244.58 | −4.644222 | uc001rzu.4 |
| ATG12 | 609608 | NA | NA | NA | NA | 756.377 | −5.710038 | uc003krh.3 |
| ATG14 | 613515 | NA | NA | NA | NA | 798.303 | −7.972395 | uc001xbw.2 |
| ATG2B | 616226 | 587.622 | −8.462493 | uc001yfi.3 | chr14: 96747595- | NA | NA | NA |
| | | | | | 96752091 | | | |
| ATG4D | 611340 | NA | NA | NA | NA | NA | NA | NA |
| ATIC | 601731 | 709.812 | −4.283927 | uc002vex.4 | chr2: 216214379- | NA | NA | NA |
| | | | | | 216214496 | | | |
| ATL2 | 609368 | 754.984 | −5.197475 | uc010ynm.2 | chr2: 38522029- | 573.499 | −3.895191 | uc010ynm.2 |
| | | | | | 38523624 | | | |
| ATM | 607585 | 646.266 | −7.148427 | uc001pkb.1 | chr11: 108236236- | NA | NA | NA |
| | | | | | 108239826 | | | |
| ATOH8 | NA | NA | NA | NA | NA | 727.169 | −5.541324 | uc002sqm.4 |
| ATP10A | 605855 | NA | NA | NA | NA | 395.584 | −4.163003 | uc010ayu.3 |
| ATP11C | 300516 | NA | NA | NA | NA | 667.511 | −7.828659 | uc004fax.3 |
| ATP13A3 | 610232 | NA | NA | NA | NA | 981.603 | −11.22549 | uc003ftx.4 |
| ATP13A4 | 609556 | NA | NA | NA | NA | 418.512 | −5.892502 | uc003ftd.3 |
| ATP1A2 | 182340 | NA | NA | NA | NA | 302.554 | −5.034847 | uc001fvb.2 |
| ATP1B2 | 182331 | NA | NA | NA | NA | 599.842 | −5.413352 | uc002gif.1 |
| ATP1B3 | 601867 | NA | NA | NA | NA | 521.92 | −7.867919 | uc003eug.1 |
| ATP1B4 | NA | NA | NA | NA | NA | 1147.99 | −10.76458 | uc004esq.3 |
| ATP2B2 | 108733 | NA | NA | NA | NA | 743.813 | −6.759495 | uc010hdp.2 |
| ATP2B4 | 108732 | NA | NA | NA | NA | 224.275 | −4.927304 | uc001gzw.3 |
| ATP2C2 | 613082 | NA | NA | NA | NA | 449.322 | −7.666387 | uc002fhx.3 |
| ATP5A1 | 164360 | NA | NA | NA | NA | 849.11 | −13.792353 | uc002lbt.1 |
| ATP5B | 102910 | 1042.86 | −8.657256 | uc001slr.3 | chr12: 57031959- | NA | NA | NA |
| | | | | | 57032106 | | | |
| ATP5C1 | 108729 | 649.173 | −5.903855 | uc010qbb.2 | chr10: 7841162- | 762.549 | −6.033502 | uc001ijv.3 |
| | | | | | 7841605 | | | |
| ATP5G1 | 603192 | 259.648 | −5.146556 | uc002iog.3 | chr17: 46973132- | 643.468 | −6.226567 | NA |
| | | | | | 46973232 | | | |
| ATP5G2 | 603193 | NA | NA | NA | NA | 569.535 | −6.486772 | uc001sec.3 |
| ATP5G3 | 602736 | NA | NA | NA | NA | 782.429 | −13.6922 | uc002ujz.4 |
| ATP5J2 | NA | NA | NA | NA | NA | 376.482 | −10.229359 | uc003uql.3 |
| ATP6AP1 | 300197 | 523.666 | −5.693356 | uc004flf.1 | chrX: 153664238- | NA | NA | NA |
| | | | | | 153664862 | | | |
| ATP6V0A2 | 611716 | 273.444 | −5.503612 | uc001ufq.1 | chr12: 124221900- | NA | NA | NA |
| | | | | | 124222049 | | | |
| ATP6V0B | 603717 | NA | NA | NA | NA | 875.694 | −5.423566 | uc001clf.3 |
| ATP6V0D1 | 607028 | 970.324 | −17.166689 | uc010vjn.1 | chr16: 67471917- | NA | NA | NA |
| | | | | | 67472430 | | | |
| ATP6V0E2 | 611019 | NA | NA | NA | NA | 515.607 | −6.06863 | uc003wgr.3 |
| ATP6V1A | 607027 | NA | NA | NA | NA | NA | NA | NA |
| ATP6V1B2 | 606939 | NA | NA | NA | NA | 962.554 | −8.872884 | uc003wzp.3 |
| ATP6V1C1 | 603097 | NA | NA | NA | NA | NA | NA | NA |
| ATP6V1C2 | NA | NA | NA | NA | NA | 853.121 | −8.0664 | uc002ras.3 |
| ATP6V1D | 609398 | NA | NA | NA | NA | 256.951 | −6.846554 | uc001xjf.3 |
| ATP6V1E1 | 108746 | NA | NA | NA | NA | NA | NA | NA |
| ATP6V1F | 607160 | NA | NA | NA | NA | 1152.61 | −5.246955 | uc003voc.2 |
| ATP6V1G1 | 607296 | 486.683 | −6.216275 | uc004bjc.3 | chr9: 117360024- | NA | NA | NA |
| | | | | | 117361152 | | | |
| ATP6V1G3 | NA | NA | NA | NA | NA | 869.114 | −1.207919 | uc009wzd.3 |
| ATP7A | 300011 | 716.68 | −1.819641 | uc004ecw.2 | chrX: 77264911- | 1105.75 | −7.025899 | uc004ecw.2 |
| | | | | | 77268087 | | | |
| ATP8B1 | 602397 | NA | NA | NA | NA | 412.713 | −5.368716 | uc002lgw.3 |
| ATP8B2 | 605867 | 588.7 | −7.016366 | uc001few.3 | chr1: 154310151- | 1214.63 | −7.809032 | uc001few.3 |
| | | | | | 154310322 | | | |
| ATP8B3 | 605866 | NA | NA | NA | NA | 472.667 | −5.698418 | uc002lty.1 |
| ATP8B4 | 609123 | 1301.08 | −9.643518 | uc001zxt.3 | chr15: 50150435- | NA | NA | NA |
| | | | | | 50152390 | | | |
| ATR | 601215 | NA | NA | NA | NA | NA | NA | NA |
| ATRNL1 | 612869 | 1077.76 | −8.421552 | uc001lcg.3 | chr10: 117704291- | 911.465 | −6.151088 | uc001lcg.3 |
| | | | | | 117708496 | | | |
| ATXN1 | 601556 | 1122.85 | −11.197959 | uc003nbt.3 | chr6: 16299343- | NA | NA | NA |
| | | | | | 16306559 | | | |
| ATXN2 | 601517 | 989.088 | −7.910787 | uc001tsh.3 | chr12: 111890018- | NA | NA | NA |
| | | | | | 111890615 | | | |
| ATXN7 | 607640 | NA | NA | NA | NA | 1391.47 | 0.013457 | uc003dlv.3 |
| ATXN7L2 | NA | NA | NA | NA | NA | 933.235 | −7.859073 | uc001dxr.3 |
| AUP1 | 602434 | 772.871 | −4.812433 | uc002sme.3 | chr2: 74753775- | 501.509 | −7.3683 | uc021vjm.1 |
| | | | | | 74753926 | | | |
| AURKB | 604970 | 1325.91 | −8.488097 | NA | NA | 812.25 | −10.995927 | uc010cnu.3 |
| AURKC | 603495 | 502.069 | −4.968085 | uc002qoc.3 | chr19: 57746786- | 1206.83 | −9.151295 | uc002qoc.3 |
| | | | | | 57746916 | | | |
| AVIL | 613397 | NA | NA | NA | NA | NA | NA | NA |
| AZI2 | 609916 | NA | NA | NA | NA | NA | NA | NA |
| B2M | 109700 | NA | NA | NA | NA | 2265.65 | −17.597682 | uc001zuc.3 |
| B3GALT1 | 603093 | 510.843 | −5.064142 | uc002udz.1 | chr2: 168726531- | 878.802 | −9.395031 | uc002udz.1 |
| | | | | | 168727366 | | | |
| B3GALT4 | 603095 | NA | NA | NA | NA | NA | NA | NA |
| B3GNT4 | 605864 | NA | NA | NA | NA | NA | NA | NA |
| B4GALNT2 | 111730 | 687.515 | −7.151568 | uc010wlt.1 | chr17: 47247091- | NA | NA | NA |
| | | | | | 47247351 | | | |
| B4GALT4 | 604015 | NA | NA | NA | NA | NA | NA | NA |
| B4GALT6 | 604017 | 214.016 | −5.651533 | uc002kwz.4 | chr18: 29202209- | NA | NA | NA |
| | | | | | 29205576 | | | |
| BABAM1 | 612766 | NA | NA | NA | NA | 579.864 | −5.030886 | uc002nfu.3 |
| BAGE3 | NA | 810.03 | −1.802787 | uc002yit.1 | chr21: 11049450- | 314.788 | −7.851233 | uc002yit.1 |
| | | | | | 11049570 | | | |
| BARD1 | 601593 | 312.698 | −9.173472 | uc010zjm.1 | chr2: 215593275- | NA | NA | NA |
| | | | | | 215593593 | | | |
| BAX | 600040 | 1110.13 | −6.56381 | uc002plf.1 | chr19: 49464355- | 677.648 | −6.681534 | uc002plj.3 |
| | | | | | 49464519 | | | |
| BAZ1B | 605681 | 431.128 | −9.307547 | uc003tyc.3 | chr7: 72854728- | NA | NA | NA |
| | | | | | 72856023 | | | |
| BAZ2A | 605682 | NA | NA | NA | NA | NA | NA | NA |
| BAZ2B | 605683 | 673.512 | −9.585952 | uc002uao.3 | chr2: 160175490- | NA | NA | NA |
| | | | | | 160176775 | | | |
| BBS4 | 600374 | NA | NA | NA | NA | 609.132 | −5.423575 | uc002avb.3 |
| BBS5 | 603650 | NA | NA | NA | NA | NA | NA | NA |
| BBS9 | 607968 | NA | NA | NA | NA | 1028.62 | −6.847433 | uc011kan.1 |
| BCAP29 | NA | NA | NA | NA | NA | 931.25 | −8.14343 | uc003vej.2 |
| BCAR3 | 604704 | NA | NA | NA | NA | 731.154 | −4.394389 | uc009wdm.1 |
| BCAR4 | 613746 | NA | NA | NA | NA | 358.675 | −5.755202 | uc010uyv.2 |
| BCAS4 | 607471 | NA | NA | NA | NA | NA | NA | NA |
| BCCIP | 611883 | NA | NA | NA | NA | 314.325 | −5.332107 | uc001ljb.4 |
| BCL10 | 603517 | NA | NA | NA | NA | NA | NA | NA |
| BCL11A | 606557 | 699.926 | −2.465276 | uc002sab.3 | chr2: 60678302- | NA | NA | NA |
| | | | | | 60679709 | | | |
| BCL2 | 151430 | 664.489 | −1.553062 | uc002lit.1 | chr18: 60790579- | NA | NA | NA |
| | | | | | 60795857 | | | |
| BCL2A1 | 601056 | NA | NA | NA | NA | NA | NA | NA |
| BCL2L11 | 603827 | 258.078 | −1.764485 | uc002tgt.1 | chr2: 111886329- | 515.498 | −1.486703 | uc002tgu.1 |
| | | | | | 111886423 | | | |
| BCL2L12 | 610837 | 768.01 | −6.755666 | uc002ppa.3 | chr19: 50177006- | NA | NA | NA |
| | | | | | 50177173 | | | |
| BCL2L14 | 606126 | 276.118 | −3.366852 | uc001rac.3 | chr12: 12251923- | 222.933 | −4.336641 | uc001rae.3 |
| | | | | | 12252627 | | | |
| BCL2L15 | NA | NA | NA | NA | NA | 354.478 | −7.584491 | uc001edw.3 |
| BCL7B | 605846 | NA | NA | NA | NA | 674.704 | −6.698824 | uc003tyf.2 |
| BCL9 | 602597 | NA | NA | NA | NA | 416.91 | −4.886935 | uc001epq.3 |
| BCLAF1 | 612588 | NA | NA | NA | NA | 785.956 | −8.790764 | uc011edb.1 |
| BCO2 | 611740 | 721.442 | −10.096836 | uc001pne.1 | chr11: 112085728- | NA | NA | NA |
| | | | | | 112086460 | | | |
| BCOR | 300485 | 731.897 | −5.237521 | uc010nhb.3 | chrX: 39910499- | NA | NA | NA |
| | | | | | 39911653 | | | |
| BCORL1 | 300688 | 491.957 | −4.805341 | uc022cdu.1 | chrX: 129190112- | NA | NA | NA |
| | | | | | 129192058 | | | |
| BCORP1 | NA | 236.897 | −2.917374 | uc011nav.1 | chrY: 21628342- | 273.478 | −3.159389 | uc011nav.1 |
| | | | | | 21628913 | | | |
| BCR | 151410 | NA | NA | NA | NA | NA | NA | NA |
| BDH1 | 603063 | NA | NA | NA | NA | NA | NA | NA |
| BDH2 | NA | NA | NA | NA | NA | NA | NA | NA |
| BDKRB2 | 113503 | NA | NA | NA | NA | 965.943 | −7.617296 | uc010avl.2 |
| BDNF | 113505 | NA | NA | NA | NA | 855.71 | −10.029552 | uc010rdu.2 |
| BDP1 | 607012 | NA | NA | NA | NA | 246.195 | −4.971255 | uc003kbn.1 |
| BEND2 | NA | NA | NA | NA | NA | 506.373 | −4.539498 | uc004cyj.4 |
| BEND7 | NA | NA | NA | NA | NA | 877.388 | −5.245182 | uc001imm.2 |
| BET1 | 605456 | NA | NA | NA | NA | NA | NA | NA |
| BEX2 | 300691 | 974.327 | −11.135713 | uc004eka.3 | chrX: 102564274- | NA | NA | NA |
| | | | | | 102564517 | | | |
| BEX4 | 300692 | 2172.07 | −13.596293 | uc004ejv.4 | chrX: 102471445- | NA | NA | NA |
| | | | | | 102472128 | | | |
| BEX5 | 300693 | 681.834 | −11.776508 | uc010nnz.3 | chrX: 101408679- | NA | NA | NA |
| | | | | | 101408901 | | | |
| BHLHB9 | 300921 | 970.057 | −7.034284 | uc004ejo.3 | chrX: 102005568- | NA | NA | NA |
| | | | | | 102007369 | | | |
| BHLHE22 | 613483 | NA | NA | NA | NA | NA | NA | NA |
| BHMT | 602888 | NA | NA | NA | NA | NA | NA | NA |
| BHMT2 | 605932 | 583.828 | −5.604559 | uc003kft.3 | chr5: 78384398- | NA | NA | NA |
| | | | | | 78385897 | | | |
| BICD1 | 602204 | NA | NA | NA | NA | NA | NA | NA |
| BIK | 603392 | NA | NA | NA | NA | 323.232 | −6.495135 | uc003bdk.3 |
| BIN3 | 606396 | NA | NA | NA | NA | NA | NA | NA |
| BIRG5 | 603352 | NA | NA | NA | NA | 208.345 | −4.748612 | uc002jvc.3 |
| BIRC7 | 605737 | NA | NA | NA | NA | 936.737 | −7.249729 | uc002yei.3 |
| BIRC8 | NA | NA | NA | NA | NA | 1105.67 | −13.275275 | uc002qbk.3 |
| BIVM | NA | 703.823 | −16.055454 | uc001vps.3 | chr13: 103492216- | NA | NA | NA |
| | | | | | 103493888 | | | |
| BLM | 604610 | 422.855 | −5.846009 | uc002bpr.3 | chr15: 91358510- | NA | NA | NA |
| | | | | | 91358686 | | | |
| BLOC1S6 | 604310 | 1632.8 | −15.269262 | uc001zvq.3 | chr15: 45898713- | 492.777 | −5.437051 | uc001zvq.3 |
| | | | | | 45901909 | | | |
| BLZF1 | 608692 | 502.157 | −7.409111 | uc001gfw.3 | chr1: 169346275- | 423.951 | −2.366946 | uc001gfx.2 |
| | | | | | 169346621 | | | |
| BMP10 | 608748 | 1251.25 | −11.708709 | uc002sez.1 | chr2: 69092613- | NA | NA | NA |
| | | | | | 69092762 | | | |
| BMP2K | NA | NA | NA | NA | NA | 214.806 | −4.712115 | uc003hlj.3 |
| BMP3 | 112263 | NA | NA | NA | NA | 906.206 | −8.728905 | uc003hmg.4 |
| BMP8B | 602284 | NA | NA | NA | NA | 1735.49 | −16.322899 | uc001cdz.1 |
| BMPR1A | 601299 | 594.033 | −9.978637 | uc001kdy.3 | chr10: 88683477- | NA | NA | NA |
| | | | | | 88684945 | | | |
| BMPR1B | 603248 | NA | NA | NA | NA | 592.382 | −5.378547 | uc003htm.4 |
| BMPR2 | 600799 | NA | NA | NA | NA | 249.417 | −8.849914 | uc002uzf.4 |
| BNIP2 | 603292 | 699.896 | −6.59091 | uc010uhb.2 | chr15: 59955062- | NA | NA | NA |
| | | | | | 59956267 | | | |
| BNIP3 | 603293 | NA | NA | NA | NA | NA | NA | NA |
| BOC | 608708 | 1210.67 | −8.025358 | uc003dzw.1 | chr3: 112969779- | 493.329 | −0.557048 | uc003dzx.3 |
| | | | | | 112970852 | | | |
| BOLA3 | 613183 | 1484.14 | −14.188083 | uc002skd.1 | chr2: 74362528- | 984.163 | −11.119094 | uc002skd.1 |
| | | | | | 74362648 | | | |
| BPHL | 603156 | 254.752 | −4.203343 | uc003muy.3 | chr6: 3152810- | 796.9 | −8.706554 | uc003muy.3 |
| | | | | | 3153432 | | | |
| BPY2B | NA | 1211.4 | −18.3382 | uc004fvj.3 | chrY: 25144416- | NA | NA | NA |
| | | | | | 25144521 | | | |
| BRD1 | 604589 | NA | NA | NA | NA | 587.304 | −6.332884 | uc011arf.2 |
| BRD3 | 601541 | 203.206 | −5.507344 | uc004cew.3 | chr9: 136895446- | NA | NA | NA |
| | | | | | 136898711 | | | |
| BRD7 | NA | 973.867 | −11.224285 | uc002ege.2 | chr16: 50352929- | 1352.95 | −10.857087 | uc002ege.2 |
| | | | | | 50353121 | | | |
| BRD9 | NA | NA | NA | NA | NA | 989.161 | −0.047985 | uc003jbl.3 |
| BRINP1 | 602865 | 603.735 | −4.430811 | uc004bkc.2 | chr9: 121928908- | NA | NA | NA |
| | | | | | 121929361 | | | |
| BRINP3 | NA | NA | NA | NA | NA | 859.799 | −8.418148 | uc010pot.1 |
| BRIX1 | NA | 2062.3 | −16.146846 | uc003jiz.3 | chr5: 34920034- | NA | NA | NA |
| | | | | | 34922014 | | | |
| BRWD1 | NA | 1117.52 | −17.496422 | uc002yxm.3 | chr21: 40668363- | NA | NA | NA |
| | | | | | 40670343 | | | |
| BSPRY | NA | NA | NA | NA | NA | 975.45 | −8.424514 | uc004bhg.4 |
| BST1 | 600387 | NA | NA | NA | NA | 310.4 | −9.760163 | uc003goh.4 |
| BTAF1 | 605191 | 1019.06 | −11.144936 | uc001khr.3 | chr10: 93788691- | NA | NA | NA |
| | | | | | 93790080 | | | |
| BTBD10 | 615933 | NA | NA | NA | NA | NA | NA | NA |
| BTBD17 | NA | 382.874 | −4.798624 | uc002jkn.2 | chr17: 72352555- | NA | NA | NA |
| | | | | | 72352795 | | | |
| BTBD7 | 610386 | 514.524 | −7.460051 | uc010aur.3 | chr14: 93703896- | 583.013 | −8.110752 | uc001ybq.4 |
| | | | | | 93708618 | | | |
| BTD | 609019 | NA | NA | NA | NA | 345.301 | −6.047635 | uc011avv.2 |
| BTF3 | 602542 | 464.992 | −8.486968 | uc010izd.1 | chr5: 72795205- | NA | NA | NA |
| | | | | | 72795476 | | | |
| BTG2 | 601597 | 717.932 | −9.776032 | uc001gzq.3 | chr1: 203276567- | NA | NA | NA |
| | | | | | 203278729 | | | |
| BTN2A2 | 613591 | 645.862 | 2.248946 | uc003nhs.3 | chr6: 26394558- | 523.827 | −4.044406 | uc003nhs.3 |
| | | | | | 26395100 | | | |
| BTN3A3 | 613595 | NA | NA | NA | NA | NA | NA | NA |
| BUB1 | 602452 | 380.956 | −6.294105 | uc002tgd.2 | chr2: 111418445- | NA | NA | NA |
| | | | | | 111419154 | | | |
| BUB1B | 602860 | 463.231 | −8.913968 | uc001zkx.4 | chr15: 40512961- | NA | NA | NA |
| | | | | | 40513337 | | | |
| BUB3 | 603719 | 1178.44 | −8.822235 | uc001lhe.2 | chr10: 124923352- | NA | NA | NA |
| | | | | | 124924886 | | | |
| BUD13 | NA | 379.807 | −6.370781 | uc009yzc.3 | chr11: 116631167- | NA | NA | NA |
| | | | | | 116631411 | | | |
| BZW2 | NA | NA | NA | NA | NA | 1141.76 | −8.528545 | uc003stn.1 |
| C10orf111 | NA | NA | NA | NA | NA | NA | NA | NA |
| C10orf131 | NA | 686.612 | −7.190301 | uc010qoo.2 | chr10: 97697967- | NA | NA | NA |
| | | | | | 97698415 | | | |
| C10orf32 | NA | NA | NA | NA | NA | 590.732 | −8.350209 | uc001kwh.2 |
| C10orf53 | NA | NA | NA | NA | NA | 408.516 | −4.76632 | uc001jib.3 |
| C10orf82 | NA | NA | NA | NA | NA | NA | NA | NA |
| C10orf88 | NA | 964.364 | −10.20894 | uc001lgw.2 | chr10: 124690419- | NA | NA | NA |
| | | | | | 124691942 | | | |
| C11orf24 | 610880 | 482.642 | −5.457377 | uc001onr.4 | chr11: 68028803- | NA | NA | NA |
| | | | | | 68029112 | | | |
| C11orf57 | NA | NA | NA | NA | NA | NA | NA | NA |
| C11orf58 | NA | NA | NA | NA | NA | NA | NA | NA |
| C11orf68 | NA | NA | NA | NA | NA | 485.316 | −7.167761 | uc001ogi.3 |
| C11orf70 | NA | 932.259 | −7.339459 | uc001pgp.3 | chr11: 101953931- | 636.676 | −10.993456 | uc001pgo.3 |
| | | | | | 101955291 | | | |
| C11orf88 | NA | NA | NA | NA | NA | 713.331 | −3.645097 | uc001plo.1 |
| C12orf29 | NA | 344.188 | −7.904757 | uc001tao.3 | chr12: 88442200- | 740.991 | −12.345314 | uc001tao.3 |
| | | | | | 88443937 | | | |
| C12orf50 | NA | 427.174 | −7.655648 | uc001tam.1 | chr12: 88373816- | 1178.62 | −7.826097 | uc001tan.3 |
| | | | | | 88374107 | | | |
| C12orf60 | NA | NA | NA | NA | NA | 1079.94 | −16.052856 | uc001rcj.4 |
| C12orf65 | 613541 | NA | NA | NA | NA | NA | NA | NA |
| C12orf73 | NA | NA | NA | NA | NA | 386.125 | −8.260024 | uc009zuj.2 |
| C14orf1 | 604576 | 1247.85 | −5.465797 | uc001xrt.3 | chr14: 76117233- | 1738.18 | −9.598907 | uc001xrt.3 |
| | | | | | 76117897 | | | |
| C14orf105 | NA | NA | NA | NA | NA | NA | NA | NA |
| C14orf159 | NA | NA | NA | NA | NA | NA | NA | NA |
| C14orf2 | 604573 | NA | NA | NA | NA | NA | NA | NA |
| C14orf28 | NA | 1163.44 | −8.819335 | uc001wvp.1 | chr14: 45373737- | 806.385 | −7.937769 | uc001wvo.3 |
| | | | | | 45376459 | | | |
| C14orf37 | NA | 252.526 | −5.129985 | uc001xdc.3 | chr14: 58470808- | 1259.64 | −6.88774 | uc001xdc.3 |
| | | | | | 58471453 | | | |
| C14orf39 | NA | NA | NA | NA | NA | 706.618 | −9.01563 | uc001xez.4 |
| C15orf32 | NA | NA | NA | NA | NA | 613.47 | −4.749643 | uc002brc.1 |
| C15orf40 | NA | NA | NA | NA | NA | 315.202 | −4.694998 | uc002bjm.3 |
| C15orf57 | NA | 1078.37 | −15.687712 | uc001zly.3 | chr15: 40823538- | NA | NA | NA |
| | | | | | 40824497 | | | |
| C15orf59 | NA | NA | NA | NA | NA | 873.177 | −12.644019 | uc002avy.3 |
| C15orf61 | NA | 1080.75 | −10.270435 | uc002aqs.3 | chr15: 67818884- | NA | NA | NA |
| | | | | | 67819641 | | | |
| C15orf65 | NA | NA | NA | NA | NA | 370.763 | −6.418252 | uc002ada.3 |
| C16orf59 | NA | 599.344 | −5.736782 | uc002cqg.2 | chr16: 2514294- | NA | NA | NA |
| | | | | | 2514964 | | | |
| C16orf62 | NA | NA | NA | NA | NA | 754.541 | −8.636282 | uc002dgm.2 |
| C16orf74 | NA | 323.84 | −3.576385 | uc002fjc.4 | chr16: 85741124- | 611.114 | −5.9016 | uc002fjc.4 |
| | | | | | 85741613 | | | |
| C16orf93 | NA | 875.194 | −10.969079 | uc002dzp.3 | chr16: 30771358- | NA | NA | NA |
| | | | | | 30771539 | | | |
| C17orf104 | NA | 603.232 | −7.1971 | uc002iha.3 | chr17: 42751565- | NA | NA | NA |
| | | | | | 42753165 | | | |
| C17orf96 | NA | NA | NA | NA | NA | 559.926 | −5.334583 | uc010wdq.2 |
| C18orf42 | 616427 | NA | NA | NA | NA | NA | NA | NA |
| C18orf63 | NA | NA | NA | NA | NA | 665.513 | −4.181136 | uc002llj.3 |
| C19orf44 | NA | NA | NA | NA | NA | NA | NA | NA |
| C19orf66 | NA | 581.16 | −3.912914 | uc002mmt.2 | chr19: 10198103- | 779.613 | −9.191134 | uc002mmt.2 |
| | | | | | 10198936 | | | |
| C1D | 606997 | NA | NA | NA | NA | 746.339 | −6.51408 | uc002sea.4 |
| C1GALT1 | 610555 | NA | NA | NA | NA | 1256.77 | −10.580497 | uc010ktm.1 |
| C1QL3 | 615227 | NA | NA | NA | NA | 1189.84 | −10.243501 | uc001ioj.1 |
| C1orf101 | NA | NA | NA | NA | NA | 1723.6 | −6.227595 | uc001ial.3 |
| C1orf112 | NA | 913.818 | −11.573 | uc010plu.1 | chr1: 169798487- | 799.924 | −11.013517 | uc009wvu.1 |
| | | | | | 169798955 | | | |
| C1orf131 | NA | NA | NA | NA | NA | 585.961 | −4.310151 | uc001hul.3 |
| C1orf140 | NA | 269.494 | −6.606572 | uc001hmw.1 | chr1: 221503270- | NA | NA | NA |
| | | | | | 221507177 | | | |
| C1orf216 | NA | 463.867 | −3.90834 | uc001bzh.1 | chr1: 36179477- | 528.395 | −7.791242 | uc001bzh.1 |
| | | | | | 36181232 | | | |
| C1orf27 | 609335 | 211.675 | −7.084357 | uc021pgg.1 | chr1: 186375281- | NA | NA | NA |
| | | | | | 186375400 | | | |
| C1orf35 | NA | NA | NA | NA | NA | 751.506 | −6.083935 | uc001hrx.3 |
| C1orf53 | NA | 762.536 | −8.348763 | uc001guh.3 | chr1: 197876351- | NA | NA | NA |
| | | | | | 197876497 | | | |
| C20orf173 | NA | NA | NA | NA | NA | NA | NA | NA |
| C21orf59 | 615494 | NA | NA | NA | NA | 260.52 | −4.945564 | uc002yqc.3 |
| C22orf39 | NA | NA | NA | NA | NA | NA | NA | NA |
| C2CD2 | NA | NA | NA | NA | NA | 311.994 | −5.774047 | uc002yzx.1 |
| C2CD3 | 615944 | 632.048 | −8.844149 | uc001ouu.2 | chr11: 73745480- | NA | NA | NA |
| | | | | | 73745642 | | | |
| C2CD4A | 610343 | NA | NA | NA | NA | NA | NA | NA |
| C2CD4B | 610344 | NA | NA | NA | NA | NA | NA | NA |
| C2CD5 | NA | NA | NA | NA | NA | NA | NA | NA |
| C2orf42 | NA | NA | NA | NA | NA | NA | NA | NA |
| C2orf43 | NA | NA | NA | NA | NA | NA | NA | NA |
| C2orf47 | NA | NA | NA | NA | NA | 794.961 | −6.809271 | uc002uvm.3 |
| C2orf49 | NA | NA | NA | NA | NA | NA | NA | NA |
| C2orf57 | NA | NA | NA | NA | NA | 227.766 | −5.388806 | uc002vrz.3 |
| C2orf72 | NA | NA | NA | NA | NA | 684.133 | −5.651462 | uc010zme.2 |
| C2orf78 | NA | 683.899 | −9.615335 | uc002sjr.1 | chr2: 74044120- | NA | NA | NA |
| | | | | | 74044274 | | | |
| C2orf80 | 615536 | 947.675 | −7.221694 | uc002vcr.3 | chr2: 209030071- | NA | NA | NA |
| | | | | | 209030530 | | | |
| C3orf22 | NA | NA | NA | NA | NA | 1286.04 | −5.694355 | uc003ejb.3 |
| C3orf30 | NA | 441.242 | −3.544189 | uc011biw.1 | chr3: 118870140- | NA | NA | NA |
| | | | | | 118870206 | | | |
| C3orf33 | NA | NA | NA | NA | NA | 1321.82 | −12.43413 | uc003fal.1 |
| C3orf36 | NA | NA | NA | NA | NA | 273.392 | −5.222337 | uc003epz.1 |
| C3orf58 | 612200 | 430.162 | −5.593289 | uc003evo.3 | chr3: 143708684- | NA | NA | NA |
| | | | | | 143711210 | | | |
| C3orf70 | NA | 525.116 | −4.553848 | uc003fpd.3 | chr3: 184795838- | 392.389 | −5.535506 | uc003fpd.3 |
| | | | | | 184800794 | | | |
| C4BPA | 120830 | 1488.15 | −15.053295 | uc001hfo.3 | chr1: 207318063- | 218.437 | −7.083614 | uc001hfo.3 |
| | | | | | 207318317 | | | |
| C4BPB | 120831 | 558.183 | −7.195297 | uc010pse.1 | chr1: 207268914- | NA | NA | NA |
| | | | | | 207269240 | | | |
| C4orf17 | NA | NA | NA | NA | NA | 1091.92 | −8.221619 | uc003huw.3 |
| C4orf26 | 614829 | NA | NA | NA | NA | 490.523 | −4.710729 | uc003hip.2 |
| C4orf27 | NA | NA | NA | NA | NA | NA | NA | NA |
| C4orf29 | NA | 1905.37 | −18.738954 | uc021xrt.1 | chr4: 128951832- | 989.319 | −6.889342 | Uc021xrt.1 |
| | | | | | 128952455 | | | |
| C5 | 120900 | 426.604 | −8.247029 | uc010mvm.1 | chr9: 123761957- | 722.454 | −9.376068 | uc004bkv.3 |
| | | | | | 123762182 | | | |
| C5AR1 | 113995 | NA | NA | NA | NA | NA | NA | NA |
| C5orf22 | NA | 834.056 | −5.690852 | uc003jhj.4 | chr5: 31553010- | NA | NA | NA |
| | | | | | 31555165 | | | |
| C5orf46 | NA | 725.764 | −5.281261 | uc003lov.4 | chr5: 147281100- | NA | NA | NA |
| | | | | | 147281187 | | | |
| C5orf56 | NA | 1411.41 | −12.310437 | uc003kwz.2 | chr5: 131811625- | NA | NA | NA |
| | | | | | 131811736 | | | |
| C5orf58 | NA | NA | NA | NA | NA | 984.201 | −10.945709 | uc010jjn.3 |
| C6orf1 | 611419 | 781.538 | −9.570472 | uc003ojf.3 | chr6: 34214157- | 332.159 | −5.703903 | uc003ojf.3 |
| | | | | | 34214290 | | | |
| C6orf132 | NA | 453.151 | −3.3676 | uc003orw.2 | chr6: 42068857- | 833.714 | −7.723433 | uc003orw.2 |
| | | | | | 42071498 | | | |
| C6orf201 | NA | NA | NA | NA | NA | 590.112 | −8.283425 | Uc011dhw.1 |
| C6orf226 | NA | NA | NA | NA | NA | 1154.51 | −9.405406 | uc003osw.3 |
| C6orf62 | NA | 742.84 | −7.617432 | uc003nel.3 | chr6: 24705090- | 1039.01 | −5.570147 | uc003nel.3 |
| | | | | | 24706364 | | | |
| C7orf31 | 616071 | 673.167 | −5.708272 | uc003sxn.1 | chr7: 25174316- | NA | NA | NA |
| | | | | | 25175590 | | | |
| C7orf34 | NA | NA | NA | NA | NA | NA | NA | NA |
| C7orf62 | NA | NA | NA | NA | NA | 369.575 | −4.406856 | uc003ujv.3 |
| C7orf73 | NA | 676.322 | −8.255527 | uc003vsy.3 | chr7: 135358914- | NA | NA | NA |
| | | | | | 135361160 | | | |
| C8orf34 | NA | 807.741 | −10.160973 | uc010lyx.2 | chr8: 69358737- | NA | NA | NA |
| | | | | | 69360245 | | | |
| C8orf48 | NA | NA | NA | NA | NA | NA | NA | NA |
| C9orf131 | NA | NA | NA | NA | NA | NA | NA | NA |
| C9orf139 | NA | NA | NA | NA | NA | NA | NA | NA |
| C9orf156 | NA | NA | NA | NA | NA | 844.025 | −7.494372 | uc022bku.1 |
| C9orf24 | NA | NA | NA | NA | NA | NA | NA | NA |
| C9orf3 | NA | NA | NA | NA | NA | 455.47 | −7.482689 | uc004aux.2 |
| C9orf47 | NA | NA | NA | NA | NA | 624.671 | −8.107551 | uc004aqc.2 |
| C9orf64 | 611342 | NA | NA | NA | NA | 569.235 | −6.841088 | uc004anb.3 |
| C9orf84 | NA | NA | NA | NA | NA | 915.006 | −9.474843 | uc004bfq.3 |
| C9orf91 | NA | NA | NA | NA | NA | 496.798 | −6.379406 | uc004bjd.4 |
| CA2 | 611492 | 203.577 | −9.116049 | uc003ydk.2 | chr8: 86393019- | NA | NA | NA |
| | | | | | 86393721 | | | |
| CA3 | 114750 | 1704.68 | −8.844461 | uc003ydj.3 | chr8: 86360383- | NA | NA | NA |
| | | | | | 86361267 | | | |
| CA5B | 300230 | NA | NA | NA | NA | 1005.16 | −5.302802 | uc004cxe.3 |
| CAB39L | 612175 | 993.047 | −10.308831 | uc001vcw.3 | chr13: 49882786- | 786.102 | −10.007189 | uc001vcw.3 |
| | | | | | 49884949 | | | |
| CABLES1 | 609194 | 554.411 | −2.606597 | uc002kub.2 | chr18: 20837332- | 292.764 | −4.497664 | uc002kub.2 |
| | | | | | 20840434 | | | |
| CABS1 | NA | 367.249 | −5.164006 | uc003hff.3 | chr4: 71201945- | NA | NA | NA |
| | | | | | 71202063 | | | |
| CACNB2 | 600003 | 2094.98 | −2.272591 | uc001ipr.2 | chr10: 18828654- | 691.997 | −2.879673 | uc001ipr.2 |
| | | | | | 18830688 | | | |
| CADM1 | 605686 | NA | NA | NA | NA | 576.467 | −6.680857 | uc001ppf.4 |
| CALCA | 114130 | NA | NA | NA | NA | NA | NA | NA |
| CALCB | 114160 | 398.563 | −5.778329 | uc001mlx.1 | chr11: 15099629- | NA | NA | NA |
| | | | | | 15100177 | | | |
| CALD1 | 114213 | NA | NA | NA | NA | NA | NA | NA |
| CALHM2 | 612235 | 696.037 | −4.181136 | uc001kxa.3 | chr10: 105206543- | NA | NA | NA |
| | | | | | 105206908 | | | |
| CALM2 | 114182 | 1372.97 | −9.290608 | uc002rvt.2 | chr2: 47387221- | 236.674 | −9.952222 | uc002rvt.2 |
| | | | | | 47387914 | | | |
| CALM3 | 114182 | 630.72 | −6.470214 | uc002pew.3 | chr19: 47112411- | NA | NA | NA |
| | | | | | 47114039 | | | |
| CAMK1 | 604998 | 301.407 | −6.98675 | uc003bst.3 | chr3: 9799029- | 1239.48 | −6.053941 | uc003bss.3 |
| | | | | | 9799222 | | | |
| CAMK2A | 114078 | NA | NA | NA | NA | 976.369 | −9.783965 | uc010jhe.2 |
| CAMK2D | 607708 | NA | NA | NA | NA | 321.463 | −5.179688 | uc003ibj.3 |
| CAMK4 | 114080 | 428.792 | −4.419144 | uc003kpf.3 | chr5: 110820165- | NA | NA | NA |
| | | | | | 110820748 | | | |
| CAMKK2 | 615002 | NA | NA | NA | NA | 668.622 | −6.917556 | uc001tzu.3 |
| CAMLG | 601118 | NA | NA | NA | NA | NA | NA | NA |
| CAMTA1 | 611501 | NA | NA | NA | NA | 321.856 | −8.101593 | uc001aoh.3 |
| CANT1 | 613165 | NA | NA | NA | NA | NA | NA | NA |
| CAP1 | NA | NA | NA | NA | NA | 740.111 | −4.140747 | uc001cey.4 |
| CAPN7 | 606400 | 471.632 | −4.712115 | uc003bzn.3 | chr3: 15292768- | NA | NA | NA |
| | | | | | 15294423 | | | |
| CAPN9 | 606401 | 279.33 | −3.754366 | uc001htz.1 | chr1: 230937343- | NA | NA | NA |
| | | | | | 230937749 | | | |
| CAPNS1 | 114170 | 321.012 | −4.041162 | uc002odi.1 | chr19: 36640728- | NA | NA | NA |
| | | | | | 36641251 | | | |
| CARD18 | 605354 | NA | NA | NA | NA | 1051.1 | −9.560057 | uc021qpy.1 |
| CASC5 | 609173 | NA | NA | NA | NA | NA | NA | NA |
| CASP1 | 147678 | 276.932 | −4.277097 | uc021qpt.1 | chr11: 104896237- | NA | NA | NA |
| | | | | | 104896327 | | | |
| CASP3 | 600636 | 944.164 | −7.969538 | uc003iwh.3 | chr4: 185548850- | 1622.03 | −7.457599 | uc003iwh.3 |
| | | | | | 185550425 | | | |
| CASP4 | 602664 | 961.837 | −7.758603 | NA | NA | 497.853 | −6.779521 | uc001pib.1 |
| CASP8 | 601763 | NA | NA | NA | NA | 344.255 | 0.185686 | uc021vuy.1 |
| CASP9 | 602234 | NA | NA | NA | NA | NA | NA | NA |
| CASS4 | NA | 314.339 | −4.990961 | uc002xxp.2 | chr20: 55033804- | NA | NA | NA |
| | | | | | 55034396 | | | |
| CAST | 114090 | 862.737 | −7.60557 | uc003klt.3 | chr5: 96108344- | NA | NA | NA |
| | | | | | 96110385 | | | |
| CATSPER2 | 607249 | 965.187 | −10.673052 | uc001zsh.3 | chr15: 43922772- | NA | NA | NA |
| | | | | | 43922898 | | | |
| CATSPERB | 611169 | 232.812 | −3.995125 | uc010aub.1 | chr14: 92047118- | 1245.5 | −13.032093 | uc010aub.1 |
| | | | | | 92047232 | | | |
| CATSPERG | 613452 | NA | NA | NA | NA | 1083.31 | −5.671908 | uc002oif.4 |
| CAV1 | 601047 | NA | NA | NA | NA | NA | NA | NA |
| CAV2 | 601048 | NA | NA | NA | NA | 845.023 | −8.459895 | uc003vid.3 |
| GBLB | 604491 | NA | NA | NA | NA | 392.771 | −5.672775 | uc003dwa.3 |
| CBLL1 | 606872 | NA | NA | NA | NA | 520.717 | −6.550353 | uc003veq.3 |
| CBLN3 | 612978 | NA | NA | NA | NA | 208.715 | −4.469281 | uc001wpg.4 |
| CBR1 | 114830 | 502.493 | −8.360496 | uc010gmx.1 | chr21: 37443496- | NA | NA | NA |
| | | | | | 37443881 | | | |
| CBR4 | NA | 991.57 | −10.475706 | uc003iry.3 | chr4: 169908742- | NA | NA | NA |
| | | | | | 169911300 | | | |
| CBWD1 | 611078 | 1371.84 | −15.02735 | uc011llr.1 | chr9: 122843- | NA | NA | NA |
| | | | | | 123162 | | | |
| CBWD2 | 611079 | 334.38 | −4.951907 | uc002tjt.2 | chr2: 114201445- | 1407.5 | −17.504002 | uc002tjt.2 |
| | | | | | 114201594 | | | |
| CBX3 | 604477 | 1242.7 | −13.305525 | uc003sxt.3 | chr7: 26251829- | 706.802 | −6.77192 | uc003sxt.3 |
| | | | | | 26253227 | | | |
| CBX5 | 604478 | 549.687 | −5.902245 | uc001sfh.4 | chr12: 54624731- | NA | NA | NA |
| | | | | | 54635538 | | | |
| CBX7 | 608457 | 231.132 | −7.162001 | uc003axb.3 | chr22: 39526779- | 1223.73 | −6.499796 | uc003axb.3 |
| | | | | | 39529895 | | | |
| CC2D2A | 612013 | 591.344 | −6.606973 | uc003gnr.4 | chr4: 15482837- | NA | NA | NA |
| | | | | | 15483887 | | | |
| CCBL2 | 610656 | 356.236 | −8.128401 | uc001dmp.2 | chr1: 89401456- | NA | NA | NA |
| | | | | | 89401865 | | | |
| CCDC108 | NA | NA | NA | NA | NA | NA | NA | NA |
| CCDC109B | NA | 1096.97 | −13.146703 | uc011cfs.2 | chr4: 110608749- | NA | NA | NA |
| | | | | | 110608872 | | | |
| CCDC110 | 609488 | NA | NA | NA | NA | 861.184 | −6.131047 | uc011ckt.1 |
| CCDC126 | NA | NA | NA | NA | NA | NA | NA | NA |
| CCDC134 | NA | 237.073 | −8.016744 | uc003bbh.1 | chr22: 42221828- | NA | NA | NA |
| | | | | | 42222303 | | | |
| CCDC144A | NA | NA | NA | NA | NA | 601.397 | −8.177433 | uc002gqk.1 |
| CCDC144NL | NA | 622.537 | −4.876265 | uc002gyf.3 | chr17: 20766708- | NA | NA | NA |
| | | | | | 20768727 | | | |
| CCDC148 | NA | 854.999 | −11.965892 | uc002tzq.3 | chr2: 159027869- | NA | NA | NA |
| | | | | | 159028624 | | | |
| CCDC150 | NA | NA | NA | NA | NA | NA | NA | NA |
| CCDC152 | NA | NA | NA | NA | NA | NA | NA | NA |
| CCDC158 | NA | NA | NA | NA | NA | 1786.65 | −5.53953 | uc003hkb.4 |
| CCDC169 | NA | 1462.49 | −3.007245 | uc010abm.3 | chr13: 36804912- | 254.548 | −5.033115 | uc010tej.2 |
| | | | | | 36805329 | | | |
| CCDC174 | NA | NA | NA | NA | NA | NA | NA | NA |
| CCDC175 | NA | NA | NA | NA | NA | NA | NA | NA |
| CCDC176 | NA | NA | NA | NA | NA | NA | NA | NA |
| CCDC18 | NA | 1151.18 | −9.145948 | uc021opx.1 | chr1: 93744035- | NA | NA | NA |
| | | | | | 93744287 | | | |
| CCDC180 | NA | 691.604 | −9.070769 | uc004axf.3 | chr9: 100105850- | NA | NA | NA |
| | | | | | 100105925 | | | |
| CCDC184 | NA | NA | NA | NA | NA | 356.192 | −5.175553 | uc001rrj.2 |
| CCDC25 | NA | NA | NA | NA | NA | NA | NA | NA |
| CCDC3 | NA | NA | NA | NA | NA | NA | NA | NA |
| CCDC34 | 612324 | 1067.64 | −12.717417 | uc001mrh.1 | chr11: 27360061- | NA | NA | NA |
| | | | | | 27360367 | | | |
| CCDC36 | NA | NA | NA | NA | NA | 282.437 | −5.495056 | uc003cwl.4 |
| CCDC58 | NA | 1044.85 | −10.599849 | uc003eey.3 | chr3: 122078436- | 893.08 | −9.078997 | uc003eey.3 |
| | | | | | 122078715 | | | |
| CCDC6 | 601985 | NA | NA | NA | NA | NA | NA | NA |
| CCDC60 | NA | 787.912 | −7.057914 | uc001txe.3 | chr12: 119978521- | NA | NA | NA |
| | | | | | 119978852 | | | |
| CCDC66 | NA | 246.676 | −4.355081 | uc003dhw.2 | chr3: 56603391- | 703.694 | −11.459369 | uc003dhw.2 |
| | | | | | 56604774 | | | |
| CCDC7 | NA | NA | NA | NA | NA | NA | NA | NA |
| CCDC70 | NA | 561.533 | −4.113323 | uc001vfu.4 | chr13: 52440217- | NA | NA | NA |
| | | | | | 52440372 | | | |
| CCDC73 | 612328 | NA | NA | NA | NA | NA | NA | NA |
| CCDC80 | 608298 | 299.126 | −4.394565 | uc011bhv.2 | chr3: 112323409- | NA | NA | NA |
| | | | | | 112324263 | | | |
| CCDC81 | NA | NA | NA | NA | NA | 1136.63 | −7.257027 | uc001pbw.2 |
| CCDC83 | NA | NA | NA | NA | NA | 875.871 | −8.843158 | uc001pbg.1 |
| CCDC88A | 609736 | 327.418 | −6.056234 | uc002ryt.2 | chr2: 55514978- | NA | NA | NA |
| | | | | | 55518300 | | | |
| CCDC88B | 611205 | NA | NA | NA | NA | 259.542 | −6.278528 | uc001nzz.1 |
| CCDC88C | 611204 | NA | NA | NA | NA | 1646.96 | −12.285305 | uc001xzi.3 |
| CCDC89 | NA | 511.248 | −5.961301 | uc001pau.1 | chr11: 85394893- | NA | NA | NA |
| | | | | | 85396048 | | | |
| CCDC9 | NA | NA | NA | NA | NA | 1227.24 | −20.191221 | uc010xym.2 |
| CCDC90B | NA | NA | NA | NA | NA | 230.497 | −3.818793 | uc001pac.3 |
| CCDC91 | NA | 496.536 | −7.487713 | uc001rio.3 | chr12: 28702107- | NA | NA | NA |
| | | | | | 28703099 | | | |
| CCDC96 | NA | NA | NA | NA | NA | 1807.38 | −13.194786 | uc003gjv.2 |
| CCK | 118440 | NA | NA | NA | NA | NA | NA | NA |
| CCL1 | 182281 | 1052.02 | −11.565785 | uc002hid.1 | chr17: 32687399- | 208.144 | −3.026875 | uc002hid.1 |
| | | | | | 32687577 | | | |
| CCL2 | 158105 | 422.435 | −4.526567 | uc002hhy.3 | chr17: 32583847- | NA | NA | NA |
| | | | | | 32584220 | | | |
| CCL20 | 601960 | 599.8 | −4.820696 | uc002vpl.2 | chr2: 228681800- | NA | NA | NA |
| | | | | | 228682280 | | | |
| CCL23 | 602494 | 817.293 | −9.385681 | uc002hks.1 | chr17: 34340097- | 359.509 | −6.165506 | NA |
| | | | | | 34340236 | | | |
| CCL25 | 602565 | NA | NA | NA | NA | 405.742 | −4.824262 | uc002mjc.4 |
| CCL5 | 187011 | 711.949 | −4.757201 | uc002hkf.3 | chr17: 34198496- | NA | NA | NA |
| | | | | | 34199380 | | | |
| CCNB1 | 123836 | 982.939 | −16.626952 | uc003jvm.3 | chr5: 68473459- | NA | NA | NA |
| | | | | | 68474070 | | | |
| CCNB2 | 602755 | NA | NA | NA | NA | 444.652 | −4.130216 | uc002afz.3 |
| CCNB3 | 300456 | NA | NA | NA | NA | NA | NA | NA |
| CCND2 | 123833 | NA | NA | NA | NA | 890.205 | −11.527604 | uc001qmo.3 |
| CCNDBP1 | 607089 | NA | NA | NA | NA | 485.131 | −6.916269 | uc001zqv.3 |
| CCNE1 | 123837 | 621.915 | −6.999868 | uc002nsn.3 | chr19: 30314685- | NA | NA | NA |
| | | | | | 30315215 | | | |
| CCNF | 600227 | NA | NA | NA | NA | 231.808 | −4.33271 | uc002cqd.1 |
| CCNG2 | 603203 | NA | NA | NA | NA | 1256.31 | −6.651738 | uc003hkq.4 |
| CCNJ | NA | NA | NA | NA | NA | NA | NA | NA |
| CCNJL | NA | NA | NA | NA | NA | NA | NA | NA |
| CCNK | 603544 | NA | NA | NA | NA | 753.23 | −7.695143 | uc001ygi.4 |
| CCNL1 | 613384 | 913.927 | −5.632346 | uc003fbe.3 | chr3: 156865586- | NA | NA | NA |
| | | | | | 156866029 | | | |
| CCNT1 | 143055 | NA | NA | NA | NA | 516.801 | −3.647738 | uc001rsd.4 |
| CCNT2 | 603862 | NA | NA | NA | NA | NA | NA | NA |
| CCNY | 612786 | NA | NA | NA | NA | 1334.86 | −7.613279 | uc001iyu.4 |
| CCR2 | 601267 | NA | NA | NA | NA | 450.802 | −11.882534 | uc003cpm.4 |
| CCRN4L | NA | 1785.59 | −17.03841 | uc003ihk.1 | chr4: 139964542- | NA | NA | NA |
| | | | | | 139965034 | | | |
| CCSER2 | NA | 1389.61 | −9.850715 | uc001kdg.1 | chr10: 86133662- | NA | NA | NA |
| | | | | | 86133761 | | | |
| CCT2 | 605139 | NA | NA | NA | NA | 213.609 | −4.956776 | uc001svb.1 |
| CCT5 | 610150 | NA | NA | NA | NA | 747.607 | −9.606266 | uc011cmq.2 |
| CCT7 | 605140 | 745.133 | −4.375428 | uc002siz.3 | chr2: 73479990- | 1517.61 | −5.779502 | uc002siz.3 |
| | | | | | 73480150 | | | |
| CCT8 | NA | NA | NA | NA | NA | 833.883 | −6.34101 | uc011acp.2 |
| CCT8L2 | NA | NA | NA | NA | NA | 360.678 | −5.649508 | uc002zlp.1 |
| CCZ1B | NA | NA | NA | NA | NA | 926.307 | −6.256458 | uc003sqy.2 |
| CD164 | 603356 | 564.233 | −10.018052 | uc003ptd.3 | chr6: 109687717- | NA | NA | NA |
| | | | | | 109689481 | | | |
| CD1A | 188370 | NA | NA | NA | NA | NA | NA | NA |
| CD1C | 188340 | NA | NA | NA | NA | 386.733 | −8.709097 | uc001fru.3 |
| CD1E | 188411 | 1028.9 | −15.661571 | uc001fry.3 | chr1: 158326687- | NA | NA | NA |
| | | | | | 158327343 | | | |
| CD200R1 | 607546 | NA | NA | NA | NA | 472.952 | −4.355516 | uc003dzj.1 |
| CD200R1L | NA | 778.193 | −7.301218 | uc003dzi.1 | chr3: 112534556- | 538.403 | −4.791895 | uc003dzi.1 |
| | | | | | 112534809 | | | |
| CD226 | 605397 | NA | NA | NA | NA | 1023.86 | −8.288795 | uc010dqo.3 |
| CD244 | 605554 | NA | NA | NA | NA | 727.559 | −8.388554 | uc001fxa.3 |
| CD2AP | 604241 | NA | NA | NA | NA | 658.235 | −12.190102 | uc003oyw.3 |
| CD2BP2 | 604470 | NA | NA | NA | NA | 784.722 | −6.793448 | uc002dxr.3 |
| CD300LG | 610520 | NA | NA | NA | NA | 559.053 | −7.32423 | uc010czk.3 |
| CD33 | 159590 | NA | NA | NA | NA | NA | NA | NA |
| CD36 | 173510 | 593.18 | −8.780672 | uc003uhf.4 | chr7: 80305700- | NA | NA | NA |
| | | | | | 80308593 | | | |
| CD40LG | 300386 | NA | NA | NA | NA | 565.894 | −8.009249 | uc004faa.3 |
| CD55 | 125240 | NA | NA | NA | NA | 1031.64 | −5.595792 | uc001hfq.4 |
| CD58 | 153420 | NA | NA | NA | NA | NA | NA | NA |
| CD69 | 107273 | 417.698 | −4.959153 | uc010sgu.2 | chr12: 9906735- | 421.463 | −5.500072 | uc001qwk.3 |
| | | | | | 9907178 | | | |
| CD72 | 107272 | NA | NA | NA | NA | 331.493 | −4.37522 | uc010mkt.1 |
| CD79A | 112205 | NA | NA | NA | NA | NA | NA | NA |
| CD80 | 112203 | NA | NA | NA | NA | 741.198 | −5.316169 | uc003ecq.3 |
| CD9 | 143030 | NA | NA | NA | NA | NA | NA | NA |
| CD99L2 | 300846 | 623.307 | −6.576836 | uc004fek.3 | chrX: 149934809- | NA | NA | NA |
| | | | | | 149937506 | | | |
| CDADC1 | NA | NA | NA | NA | NA | NA | NA | NA |
| CDC123 | NA | NA | NA | NA | NA | NA | NA | NA |
| CDC14A | 603504 | 507.379 | −5.416632 | uc001dtg.4 | chr1: 100983837- | 1552.26 | −4.079213 | uc001dte.4 |
| | | | | | 100985833 | | | |
| CDC16 | 603461 | 358.531 | −7.053353 | uc001vuk.1 | chr13: 115038039- | NA | NA | NA |
| | | | | | 115038150 | | | |
| CDC20 | 603618 | NA | NA | NA | NA | 651.744 | −7.763979 | uc001cix.3 |
| CDC23 | 603462 | 631.528 | −6.75899 | uc003lcm.1 | chr5: 137541563- | NA | NA | NA |
| | | | | | 137542151 | | | |
| CDC37L1 | 610346 | NA | NA | NA | NA | 726.828 | −6.142166 | uc003zio.3 |
| CDC40 | 605585 | NA | NA | NA | NA | NA | NA | NA |
| CDC42SE2 | NA | NA | NA | NA | NA | NA | NA | NA |
| CDC5L | 602868 | NA | NA | NA | NA | NA | NA | NA |
| CDC7 | 603311 | NA | NA | NA | NA | NA | NA | NA |
| CDCA4 | 612270 | NA | NA | NA | NA | 500.71 | −8.741178 | uc001yqa.2 |
| CDCA5 | 609374 | 1071.32 | −8.199776 | uc001ocp.2 | chr11: 64844927- | NA | NA | NA |
| | | | | | 64846578 | | | |
| CDCA7L | 609685 | NA | NA | NA | NA | 743.751 | −7.947492 | uc003sve.4 |
| CDCP1 | 611735 | 545.54 | −4.271943 | uc003con.3 | chr3: 45151731- | 1376.01 | −10.049214 | uc003com.3 |
| | | | | | 45151956 | | | |
| CDH1 | 192090 | NA | NA | NA | NA | 712.676 | −8.464332 | uc002ewg.1 |
| CDH13 | 601364 | NA | NA | NA | NA | 230.759 | −4.014158 | uc021tlw.1 |
| CDH17 | 603017 | NA | NA | NA | NA | 580.898 | −8.462248 | uc003ygh.2 |
| CDH18 | 603019 | NA | NA | NA | NA | 209.43 | −5.49047 | uc021xwu.1 |
| CDH23 | 605516 | NA | NA | NA | NA | 575.193 | −0.787664 | uc009xql.3 |
| CDH5 | 601120 | NA | NA | NA | NA | 690.788 | −6.313894 | uc002eon.1 |
| CDH6 | 603007 | 915.634 | −6.143871 | uc003jhe.2 | chr5: 31323416- | NA | NA | NA |
| | | | | | 31329253 | | | |
| CDH7 | 605806 | 1151.49 | −11.828811 | uc002lka.3 | chr18: 63530183- | 310.473 | −7.042182 | uc002ljz.3 |
| | | | | | 63531361 | | | |
| CDH8 | 603008 | 968.8 | −6.639862 | uc002eog.2 | chr16: 61685915- | NA | NA | NA |
| | | | | | 61687511 | | | |
| CDK1 | 116940 | 726.739 | −21.285142 | uc010qii.2 | chr10: 62545558- | 1698.74 | −19.560287 | uc001jld.3 |
| | | | | | 62547988 | | | |
| CDK13 | 603309 | NA | NA | NA | NA | 493.739 | −5.015324 | uc003thh.4 |
| CDK17 | 603440 | NA | NA | NA | NA | 297.15 | −9.131762 | uc001tep.2 |
| CDK5RAP1 | 608200 | 777.293 | −14.948345 | uc002wzb.1 | chr20: 31946647- | NA | NA | NA |
| | | | | | 31948114 | | | |
| CDKL3 | 608459 | 574.306 | −3.568908 | uc011cxm.1 | chr5: 133541305- | 452.516 | −3.26535 | uc011cxm.1 |
| | | | | | 133541558 | | | |
| CDKL4 | NA | 781.421 | −7.327679 | uc002rrm.3 | chr2: 39405688- | NA | NA | NA |
| | | | | | 39406306 | | | |
| CDKN1A | 116899 | NA | NA | NA | NA | 1718.27 | −18.614253 | uc021yzb.1 |
| CDKN1B | 600778 | NA | NA | NA | NA | NA | NA | NA |
| CDKN1C | 600856 | NA | NA | NA | NA | 459.963 | −4.006339 | uc001lwr.4 |
| CDKN2AIP | 615914 | 344.867 | −6.648004 | uc003ivp.1 | chr4: 184368581- | NA | NA | NA |
| | | | | | 184369049 | | | |
| CDKN2AIPNL | NA | NA | NA | NA | NA | NA | NA | NA |
| CDKN2C | 603369 | NA | NA | NA | NA | NA | NA | NA |
| CDON | 608707 | NA | NA | NA | NA | 560.794 | −8.852866 | uc001qdb.4 |
| CDR2 | 117340 | NA | NA | NA | NA | NA | NA | NA |
| CDSN | 602593 | NA | NA | NA | NA | 561.92 | −7.935349 | uc011fzy.2 |
| CDV3 | NA | NA | NA | NA | NA | 387.421 | −9.117455 | uc003epp.4 |
| CDX1 | 600746 | NA | NA | NA | NA | NA | NA | NA |
| CEACAM1 | 109770 | NA | NA | NA | NA | 633.407 | −6.420475 | uc002otv.3 |
| CEACAM21 | NA | 204.249 | −5.409518 | uc002ore.4 | chr19: 42092817- | NA | NA | NA |
| | | | | | 42093197 | | | |
| CEACAM4 | NA | NA | NA | NA | NA | 236.094 | −5.175553 | uc002orh.1 |
| CEACAM5 | 114890 | 234.617 | −4.056795 | NA | NA | 767.203 | −4.465697 | uc010ehz.1 |
| CEACAM8 | 615747 | NA | NA | NA | NA | 758.707 | −6.293504 | uc002oud.2 |
| CEBPZ | 612828 | 546.142 | −11.658577 | uc002rpz.3 | chr2: 37428775- | NA | NA | NA |
| | | | | | 37428906 | | | |
| CELA3B | NA | NA | NA | NA | NA | 307.151 | −4.98961 | uc001bfk.3 |
| CELF5 | 612680 | 775.281 | −7.753551 | uc002lxl.2 | chr19: 3293333- | NA | NA | NA |
| | | | | | 3297073 | | | |
| CELSR2 | 604265 | NA | NA | NA | NA | 344.952 | −6.449887 | uc001dxa.4 |
| CENPC | 117141 | NA | NA | NA | NA | 640.978 | −6.406897 | uc003hdd.1 |
| CENPE | 117143 | 414.884 | −4.519712 | uc003hxb.1 | chr4: 104026963- | NA | NA | NA |
| | | | | | 104027378 | | | |
| CENPH | 605607 | NA | NA | NA | NA | 1471.61 | −12.70313 | uc003jvp.3 |
| CENPQ | 611506 | NA | NA | NA | NA | 323.6 | −9.566223 | uc003ozh.1 |
| CENPV | 608139 | 570.276 | −7.504018 | uc002gpw.3 | chr17: 16245848- | NA | NA | NA |
| | | | | | 16246130 | | | |
| CENPW | 611264 | NA | NA | NA | NA | NA | NA | NA |
| CEP120 | 613446 | NA | NA | NA | NA | 1543.12 | −14.611696 | uc003ktk.3 |
| CEP135 | 611423 | 400.241 | −4.242707 | uc010igz.1 | chr4: 56832053- | NA | NA | NA |
| | | | | | 56832746 | | | |
| CEP152 | 613529 | 1317.79 | −11.427382 | uc001zxa.2 | chr15: 49033345- | NA | NA | NA |
| | | | | | 49033783 | | | |
| CEP162 | 610201 | NA | NA | NA | NA | 521.028 | −4.634588 | uc003pki.4 |
| CEP164 | 614848 | 350.97 | −4.852798 | uc001prg.1 | chr11: 117280846- | NA | NA | NA |
| | | | | | 117281417 | | | |
| CEP170 | 613023 | 1270.69 | −7.950231 | uc021pls.1 | chr1: 243374515- | NA | NA | NA |
| | | | | | 243375032 | | | |
| CEP290 | 610142 | 498.579 | −7.830195 | uc001taq.3 | chr12: 88442790- | NA | NA | NA |
| | | | | | 88442960 | | | |
| CEP44 | NA | 608.935 | −12.850309 | uc003itr.3 | chr4: 175238535- | 1857.28 | −5.245797 | uc003itr.3 |
| | | | | | 175241482 | | | |
| CEP55 | 610000 | NA | NA | NA | NA | 606.546 | −8.696259 | uc009xug.3 |
| CEP57 | 607951 | NA | NA | NA | NA | 445.18 | −4.485099 | uc001pfo.2 |
| CEP57L1 | NA | 409.887 | −5.972452 | uc003psx.4 | chr6: 109483953- | NA | NA | NA |
| | | | | | 109485115 | | | |
| CEP63 | 614724 | 639.376 | −9.657021 | uc003eql.1 | chr3: 134280378- | 861.192 | −2.406554 | uc003eqm.3 |
| | | | | | 134283870 | | | |
| CEP70 | 614310 | NA | NA | NA | NA | 1567.14 | −5.86213 | uc003esl.3 |
| CEP72 | 616475 | NA | NA | NA | NA | 993.094 | −8.442764 | uc003jbf.3 |
| CEP78 | NA | 823.232 | −5.211948 | uc011lsp.1 | chr9: 80863834- | 460.016 | −5.354181 | uc004aky.4 |
| | | | | | 80865369 | | | |
| CEP83 | 615847 | 866.274 | −8.158815 | uc001tdd.3 | chr12: 94702056- | NA | NA | NA |
| | | | | | 94702588 | | | |
| CEP89 | 615470 | 498.939 | −8.452835 | uc002nua.3 | chr19: 33422133- | NA | NA | NA |
| | | | | | 33422292 | | | |
| CEP97 | 615864 | 519.491 | −6.749008 | uc003dvk.1 | chr3: 101484396- | NA | NA | NA |
| | | | | | 101486181 | | | |
| CERS3 | 615276 | NA | NA | NA | NA | 423.489 | −4.174426 | uc002bvz.3 |
| CES1P1 | NA | NA | NA | NA | NA | 245.243 | −6.07173 | uc002eik.3 |
| CES5A | NA | NA | NA | NA | NA | 736.716 | −5.814373 | uc002eio.2 |
| CETN1 | 603187 | NA | NA | NA | NA | 973.356 | −9.016836 | uc002kko.1 |
| CETN2 | 300006 | 530.907 | −4.054336 | uc004fgq.3 | chrX: 151995871- | 507.036 | −6.482968 | uc004fgq.3 |
| | | | | | 151996384 | | | |
| CETN3 | 602907 | 923.371 | −12.426121 | uc003kjo.3 | chr5: 89689529- | NA | NA | NA |
| | | | | | 89689880 | | | |
| CFAP20 | NA | NA | NA | NA | NA | 1961.08 | −16.308774 | uc002enb.1 |
| CFAP43 | NA | 646.88 | −8.260514 | uc009xxq.3 | chr10: 105889646- | 1455.09 | −9.327977 | uc009xxq.3 |
| | | | | | 105889896 | | | |
| CFAP44 | NA | NA | NA | NA | NA | 349.138 | −8.93029 | uc010hqj.2 |
| CFAP45 | 605152 | NA | NA | NA | NA | 306.878 | −2.830685 | uc001fui.3 |
| CFAP47 | NA | 1148.55 | −6.189839 | uc004ddk.1 | chrX: 36162927- | 215.568 | −3.607941 | uc004ddk.1 |
| | | | | | 36163187 | | | |
| CFAP53 | 614759 | 1683.34 | −11.128728 | uc002lee.2 | chr18: 47753563- | 1550.93 | −17.353677 | uc002lee.2 |
| | | | | | 47753750 | | | |
| CFAP61 | NA | NA | NA | NA | NA | 691.894 | −9.128699 | uc002wru.3 |
| CFAP70 | NA | 272.509 | −3.873459 | uc009xrd.1 | chr10: 75053021- | NA | NA | NA |
| | | | | | 75056781 | | | |
| CFAP97 | 616047 | 1257.04 | −9.015977 | uc003ixg.3 | chr4: 186090180- | NA | NA | NA |
| | | | | | 186091452 | | | |
| CFH | 134370 | NA | NA | NA | NA | 1419.08 | −19.118384 | uc001gtj.4 |
| CFHR1 | 134371 | NA | NA | NA | NA | 254.801 | −10.917978 | uc001gtm.3 |
| CFHR2 | 600889 | NA | NA | NA | NA | NA | NA | NA |
| CFHR3 | 605336 | 313.428 | −9.7788 | uc001gtk.3 | chr1: 196757961- | NA | NA | NA |
| | | | | | 196759152 | | | |
| CFHR5 | 608593 | 845.421 | −10.595342 | uc001gts.4 | chr1: 196977814- | 781.599 | −9.32009 | uc001gts.4 |
| | | | | | 196978803 | | | |
| CFI | 217030 | NA | NA | NA | NA | 1020.25 | −12.326294 | uc003hzq.3 |
| CFP | 300383 | NA | NA | NA | NA | NA | NA | NA |
| CGA | 118850 | NA | NA | NA | NA | NA | NA | NA |
| CGGBP1 | 603363 | 1539.59 | −12.990028 | uc003dqs.3 | chr3: 88101100- | NA | NA | NA |
| | | | | | 88104622 | | | |
| CHAC2 | NA | NA | NA | NA | NA | 554.243 | −9.118596 | uc002rxk.1 |
| CHCHD4 | 611077 | NA | NA | NA | NA | NA | NA | NA |
| CHD1 | 602118 | NA | NA | NA | NA | NA | NA | NA |
| CHD2 | 602119 | 613.999 | −1.558496 | uc002bsn.3 | chr15: 93492311- | 648 | −1.123285 | uc002bsm.2 |
| | | | | | 93492463 | | | |
| CHD6 | 616114 | 708.649 | −11.616953 | uc002xjz.1 | chr20: 40031170- | 1242.95 | −14.036454 | uc002xkb.1 |
| | | | | | 40033232 | | | |
| CHD8 | 610528 | NA | NA | NA | NA | NA | NA | NA |
| CHEK2 | 604373 | 1503.82 | −9.546017 | uc003ads.1 | chr22: 29083731- | NA | NA | NA |
| | | | | | 29083884 | | | |
| CHGB | 118920 | NA | NA | NA | NA | NA | NA | NA |
| CHIC2 | 604332 | NA | NA | NA | NA | 1502.2 | −15.369036 | uc003haj.2 |
| CHKA | 118491 | NA | NA | NA | NA | NA | NA | NA |
| CHMP2B | 609512 | 895.543 | −11.330334 | uc003dqp.4 | chr3: 87302973- | NA | NA | NA |
| | | | | | 87304698 | | | |
| CHMP4A | 610051 | NA | NA | NA | NA | 565.933 | −5.353179 | uc001wni.3 |
| CHMP6 | 610901 | 342.1 | −4.990321 | uc002jyw.4 | chr17: 78972954- | NA | NA | NA |
| | | | | | 78973933 | | | |
| CHMP7 | 611130 | 698.856 | −8.565907 | uc003xdc.2 | chr8: 23118113- | NA | NA | NA |
| | | | | | 23119512 | | | |
| CHODL | 607247 | NA | NA | NA | NA | 919.532 | −10.61904 | uc002ykr.3 |
| CHORDC1 | 604353 | NA | NA | NA | NA | NA | NA | NA |
| CHPT1 | NA | 318.195 | −4.62131 | uc001tip.1 | chr12: 102120204- | 936.498 | −10.341375 | uc001tin.3 |
| | | | | | 102123424 | | | |
| CHRDL2 | 613127 | 588.828 | −6.879416 | NA | NA | 617.653 | −6.879416 | uc001ovg.3 |
| CHRM3 | 118494 | 431.187 | −6.899372 | uc001hyp.3 | chr1: 240072525- | NA | NA | NA |
| | | | | | 240072717 | | | |
| CHRM5 | 118496 | 632.539 | −8.422437 | uc001zhk.1 | chr15: 34356518- | NA | NA | NA |
| | | | | | 34357287 | | | |
| CHRNG | 100730 | 355.101 | −9.41261 | uc010fyd.3 | chr2: 233406244- | NA | NA | NA |
| | | | | | 233406592 | | | |
| CHST11 | 610128 | NA | NA | NA | NA | 957.582 | −14.80168 | uc001tky.3 |
| CHST12 | 610129 | NA | NA | NA | NA | NA | NA | NA |
| CHST8 | 610190 | NA | NA | NA | NA | NA | NA | NA |
| CHSY1 | 608183 | 204.234 | −5.671791 | uc010usd.2 | chr15: 101715928- | NA | NA | NA |
| | | | | | 101717592 | | | |
| CHSY3 | 609963 | 1627.88 | −20.267391 | uc003kvd.3 | chr5: 129521485- | NA | NA | NA |
| | | | | | 129522327 | | | |
| CHTF8 | 613202 | NA | NA | NA | NA | NA | NA | NA |
| CHTOP | 614206 | NA | NA | NA | NA | 223.989 | −5.118764 | uc009woj.2 |
| CHURC1 | 608577 | 980.505 | −10.700957 | uc001xhw.2 | chr14: 65398949- | NA | NA | NA |
| | | | | | 65402084 | | | |
| CIAPIN1 | 608943 | 279.202 | −4.548486 | uc002ell.1 | chr16: 57462087- | NA | NA | NA |
| | | | | | 57463081 | | | |
| CIDEA | 604440 | NA | NA | NA | NA | 658.294 | −5.459286 | uc002kqt.4 |
| CILP | 603489 | 674.424 | −6.782484 | uc002aon.2 | chr15: 65488337- | NA | NA | NA |
| | | | | | 65489068 | | | |
| CIPC | NA | NA | NA | NA | NA | NA | NA | NA |
| CIR1 | 605228 | 564.625 | −6.230108 | uc002uim.3 | chr2: 175212878- | NA | NA | NA |
| | | | | | 175213224 | | | |
| CIRH1A | NA | 528.044 | −8.323079 | uc002ewr.2 | chr16: 69199475- | NA | NA | NA |
| | | | | | 69201745 | | | |
| CISD2 | 611507 | NA | NA | NA | NA | 609.294 | −6.412487 | uc003hwt.4 |
| CKAP2 | 611569 | NA | NA | NA | NA | 911.074 | −9.427165 | uc001vgu.2 |
| CKLF | 616074 | NA | NA | NA | NA | 1996.04 | −19.48529 | uc002eot.3 |
| CKMT1B | 123290 | NA | NA | NA | NA | 665.823 | −11.567842 | uc010udv.1 |
| CKS1B | 116900 | NA | NA | NA | NA | 523.057 | −4.766854 | uc001fgb.3 |
| CKS2 | 116901 | 526.624 | −10.127995 | uc004aqh.3 | chr9: 91931341- | NA | NA | NA |
| | | | | | 91931618 | | | |
| CLASP1 | 605852 | 1207.02 | −10.223343 | uc002tnf.3 | chr2: 122121706- | 828.264 | −6.22512 | uc002tmy.3 |
| | | | | | 122122627 | | | |
| CLASP2 | 605853 | 513.861 | −5.11101 | uc003cfv.3 | chr3: 33643935- | 932.203 | −11.040496 | uc003cfs.3 |
| | | | | | 33644028 | | | |
| CLCA1 | 603906 | NA | NA | NA | NA | NA | NA | NA |
| CLCN3 | 600580 | 855.927 | −1.593137 | uc003ish.3 | chr4: 170641217- | 457.453 | −4.264037 | uc003ish.3 |
| | | | | | 170644338 | | | |
| CLDN11 | 601326 | 601.321 | −0.968871 | uc003fgx.3 | chr3: 170150545- | NA | NA | NA |
| | | | | | 170152479 | | | |
| CLDN14 | 605608 | NA | NA | NA | NA | 871.309 | −8.593999 | uc002yvl.1 |
| CLDN22 | NA | 650.25 | −7.216864 | uc010isa.1 | chr4: 184239220- | NA | NA | NA |
| | | | | | 184240708 | | | |
| CLDN6 | 615798 | NA | NA | NA | NA | 1394.76 | −8.594202 | uc002csu.4 |
| CLDN7 | 609131 | NA | NA | NA | NA | 579.84 | −4.062675 | uc002gfm.4 |
| CLDN8 | 611231 | NA | NA | NA | NA | 333.26 | −6.844158 | uc002ynu.2 |
| CLDND1 | NA | 488.627 | −11.309752 | uc003dso.2 | chr3: 98225321- | 933.307 | −8.992724 | uc003dsp.3 |
| | | | | | 98225577 | | | |
| CLEC18B | 616572 | 1126.38 | −5.070206 | uc010vmu.1 | chr16: 74444434- | NA | NA | NA |
| | | | | | 74445938 | | | |
| CLEC4A | 605306 | NA | NA | NA | NA | 1137.73 | −5.702827 | uc001qtz.1 |
| CLEC4D | 609964 | NA | NA | NA | NA | NA | NA | NA |
| CLEC6A | 613579 | NA | NA | NA | NA | NA | NA | NA |
| CLEC9A | 612252 | 371.832 | −4.45596 | uc001qxa.3 | chr12: 10218232- | NA | NA | NA |
| | | | | | 10218565 | | | |
| CLECL1 | 607467 | NA | NA | NA | NA | 523.918 | −4.53937 | uc001qwj.3 |
| CLIC2 | 300138 | NA | NA | NA | NA | 1015.41 | −8.940642 | uc004fnf.3 |
| CLIC6 | 615321 | 704.263 | −6.093815 | uc002yuf.1 | chr21: 36088781- | NA | NA | NA |
| | | | | | 36090519 | | | |
| CLK1 | 601951 | NA | NA | NA | NA | 272.411 | −4.995621 | uc010zhi.1 |
| CLK2 | 602989 | NA | NA | NA | NA | 1472.71 | −6.687955 | uc001fjw.3 |
| CLK2P1 | NA | NA | NA | NA | NA | 673.943 | −7.276272 | uc003swk.2 |
| CLK3 | 602990 | 377.082 | −2.79134 | uc010ulm.1 | chr15: 74912603- | NA | NA | NA |
| | | | | | 74914557 | | | |
| CLK4 | 607969 | 338.586 | −11.463417 | uc003mjf.1 | chr5: 178029665- | 1780.39 | −6.703617 | uc011dgg.1 |
| | | | | | 178030617 | | | |
| CLMN | 611121 | 832.831 | −6.091105 | uc001yef.2 | chr14: 95648276- | NA | NA | NA |
| | | | | | 95657900 | | | |
| CLMP | 611693 | NA | NA | NA | NA | NA | NA | NA |
| CLN5 | 608102 | 1330.34 | −7.615349 | uc001vkc.3 | chr13: 77575105- | NA | NA | NA |
| | | | | | 77576652 | | | |
| CLNK | 611434 | NA | NA | NA | NA | 970.555 | −0.15257 | uc003gmo.4 |
| CLPB | 616254 | NA | NA | NA | NA | NA | NA | NA |
| CLRN1 | 606397 | 247.397 | −4.792627 | uc003eyk.1 | chr3: 150644354- | 312.126 | −5.109962 | uc003eyj.3 |
| | | | | | 150645722 | | | |
| CLRN3 | NA | 838.318 | −11.115052 | uc001ljz.1 | chr10: 129676114- | NA | NA | NA |
| | | | | | 129676412 | | | |
| CLTA | 118960 | 840.252 | −10.553577 | uc003zzc.3 | chr9: 36211772- | NA | NA | NA |
| | | | | | 36212059 | | | |
| CMC2 | NA | 837.407 | −9.294135 | uc002ffu.3 | chr16: 81009699- | NA | NA | NA |
| | | | | | 81009989 | | | |
| CMPK | 191710 | NA | NA | NA | NA | NA | NA | NA |
| CMSS1 | NA | 408.802 | −10.612987 | uc003dtl.3 | chr3: 99897233- | NA | NA | NA |
| | | | | | 99897476 | | | |
| CMTM2 | 607885 | 1074.81 | −8.795019 | uc002ept.3 | chr16: 66622023- | NA | NA | NA |
| | | | | | 66622177 | | | |
| CMTR2 | 616190 | NA | NA | NA | NA | NA | NA | NA |
| CNGA1 | 123825 | NA | NA | NA | NA | NA | NA | NA |
| CNIH1 | 611287 | NA | NA | NA | NA | NA | NA | NA |
| CNKSR3 | NA | 836.271 | −5.98004 | uc003qpy.3 | chr6: 154726433- | NA | NA | NA |
| | | | | | 154727487 | | | |
| CNN2 | 602373 | NA | NA | NA | NA | 1220.56 | −5.652832 | uc002lqu.3 |
| CNN3 | 602374 | 1331.88 | −10.230977 | uc010otv.2 | chr1: 95362507- | NA | NA | NA |
| | | | | | 95363297 | | | |
| CNOT4 | 604911 | NA | NA | NA | NA | 981.966 | −6.820699 | uc003vsu.2 |
| CNOT6L | NA | 716.164 | −5.268188 | uc011ccd.2 | chr4: 78634541- | 817.353 | −3.320933 | uc011ccd.2 |
| | | | | | 78641584 | | | |
| CNOT7 | 604913 | 728.762 | −8.410521 | uc003wxh.1 | chr8: 17089022- | NA | NA | NA |
| | | | | | 17089929 | | | |
| CNP | 123830 | 605.309 | −7.074092 | uc002hyl.1 | chr17: 40125943- | NA | NA | NA |
| | | | | | 40129754 | | | |
| CNPY1 | 612493 | NA | NA | NA | NA | 661.797 | −7.525995 | uc003wmc.1 |
| CNPY2 | 605861 | 611.272 | −6.703869 | uc001sku.2 | chr12: 56704213- | NA | NA | NA |
| | | | | | 56704335 | | | |
| CNRIP1 | NA | 993.001 | −4.732762 | uc002sej.4 | chr2: 68511303- | NA | NA | NA |
| | | | | | 68511559 | | | |
| CNTD2 | NA | NA | NA | NA | NA | 356.694 | −5.216751 | uc010xvi.2 |
| CNTF | 118945 | 412.908 | −6.153975 | uc001nna.4 | chr11: 58391996- | NA | NA | NA |
| | | | | | 58393205 | | | |
| CNTLN | 611870 | 309.08 | −3.555114 | uc003zmx.4 | chr9: 17298381- | NA | NA | NA |
| | | | | | 17302049 | | | |
| CNTN3 | 601325 | NA | NA | NA | NA | 483.329 | −6.549115 | uc003dpm.1 |
| CNTN4 | 607280 | NA | NA | NA | NA | 402.166 | −3.441979 | uc003bpc.3 |
| CNTN5 | 607219 | NA | NA | NA | NA | 1088.47 | −13.388474 | uc009ywv.2 |
| CNTN6 | 607220 | NA | NA | NA | NA | 1410.95 | −18.775715 | uc010hbo.2 |
| CNTRL | 605496 | 217.914 | −10.875735 | uc004bkx.1 | chr9: 123939465- | NA | NA | NA |
| | | | | | 123939886 | | | |
| CNTROB | 611425 | 858.771 | −4.193113 | uc002gjp.3 | chr17: 7852829- | NA | NA | NA |
| | | | | | 7853237 | | | |
| COA5 | 613920 | NA | NA | NA | NA | NA | NA | NA |
| COBLL1 | 610318 | NA | NA | NA | NA | NA | NA | NA |
| COG4 | 606976 | NA | NA | NA | NA | 429.383 | −4.548676 | uc002ezc.3 |
| COL12A1 | 120320 | NA | NA | NA | NA | NA | NA | NA |
| COL13A1 | 120350 | NA | NA | NA | NA | NA | NA | NA |
| COL19A1 | 120165 | 1227.28 | −8.390266 | uc003pfc.1 | chr6: 70916979- | NA | NA | NA |
| | | | | | 70922157 | | | |
| COL21A1 | 610002 | NA | NA | NA | NA | NA | NA | NA |
| COL23A1 | 610043 | NA | NA | NA | NA | NA | NA | NA |
| COL3A1 | 120180 | 505.905 | −6.246893 | uc002uqj.1 | chr2: 189876501- | NA | NA | NA |
| | | | | | 189877472 | | | |
| COL4A1 | 120130 | NA | NA | NA | NA | NA | NA | NA |
| COL4A2 | 120090 | NA | NA | NA | NA | NA | NA | NA |
| COL4A3 | 120070 | NA | NA | NA | NA | NA | NA | NA |
| COL4A3BP | 604677 | NA | NA | NA | NA | NA | NA | NA |
| COL6A3 | 120250 | 699.003 | −6.884633 | uc002vwj.2 | chr2: 238232655- | NA | NA | NA |
| | | | | | 238233416 | | | |
| COLEC10 | 607620 | 592.596 | −6.445321 | uc003yoo.3 | chr8: 120118431- | 240.672 | −3.155209 | uc003yoo.3 |
| | | | | | 120119202 | | | |
| COLGALT2 | NA | 1245.74 | −7.712518 | uc010poj.1 | chr1: 183898796- | 725.039 | −8.476284 | uc001gqp.3 |
| | | | | | 183899362 | | | |
| COMMD10 | NA | NA | NA | NA | NA | NA | NA | NA |
| COMMD6 | 612377 | 294.866 | −7.309197 | uc001vjn.1 | chr13: 76099350- | NA | NA | NA |
| | | | | | 76100724 | | | |
| COMMD8 | 616656 | NA | NA | NA | NA | NA | NA | NA |
| COPB2 | 606990 | 910.078 | −5.81851 | uc003etg.3 | chr3: 139098867- | NA | NA | NA |
| | | | | | 139101971 | | | |
| COPS4 | 616008 | NA | NA | NA | NA | 949.115 | −10.491934 | uc003hoa.3 |
| COPS8 | 616011 | 770.974 | −8.785368 | uc002vwg.3 | chr2: 238006386- | NA | NA | NA |
| | | | | | 238007489 | | | |
| COQ10B | NA | NA | NA | NA | NA | 617.721 | −5.95853 | uc002uuh.1 |
| COQ2 | 609825 | NA | NA | NA | NA | NA | NA | NA |
| COQ3 | 605196 | NA | NA | NA | NA | 1001.08 | −9.764987 | uc003ppk.3 |
| COQ4 | 612898 | NA | NA | NA | NA | 623.322 | −7.940053 | uc004bur.4 |
| CORIN | 605236 | NA | NA | NA | NA | 1500.88 | −13.674753 | uc011bzi.1 |
| CORO2A | 602159 | NA | NA | NA | NA | NA | NA | NA |
| COX10 | 602125 | NA | NA | NA | NA | 464.417 | −8.741827 | uc002gof.4 |
| COX16 | NA | NA | NA | NA | NA | 684.056 | −6.092115 | uc001xmb.3 |
| COX7A2 | 123996 | 663.645 | −8.130103 | uc003phv.2 | chr6: 75947391- | NA | NA | NA |
| | | | | | 75947645 | | | |
| COX7A2L | 605771 | 204.652 | −3.172767 | uc002rsk.3 | chr2: 42577645- | 451.902 | −4.477337 | uc002rsk.3 |
| | | | | | 42578358 | | | |
| COX7B2 | 609811 | NA | NA | NA | NA | NA | NA | NA |
| COX8C | NA | 479.981 | −4.487595 | uc001ybt.1 | chr14: 93814467- | NA | NA | NA |
| | | | | | 93814700 | | | |
| CP | 117700 | 933.168 | −11.689376 | uc010hvf.1 | chr3: 148903853- | NA | NA | NA |
| | | | | | 148904292 | | | |
| CPA2 | 600688 | NA | NA | NA | NA | NA | NA | NA |
| CPA6 | 609562 | NA | NA | NA | NA | 610.485 | −6.142054 | uc003xxs.2 |
| CPAMD8 | 608841 | NA | NA | NA | NA | 285.801 | −5.810792 | uc002nfb.3 |
| CPB1 | 114852 | 1700.2 | −7.647771 | uc003ewl.3 | chr3: 148577790- | NA | NA | NA |
| | | | | | 148577972 | | | |
| CPB2 | 603101 | NA | NA | NA | NA | NA | NA | NA |
| CPEB4 | 610607 | 1066.84 | −7.175414 | uc010jju.2 | chr5: 173380309- | NA | NA | NA |
| | | | | | 173383823 | | | |
| CPLX2 | 605033 | 1018.77 | −9.117283 | uc003mde.1 | chr5: 175307049- | 1057.86 | −6.538364 | uc003mde.1 |
| | | | | | 175311023 | | | |
| CPLX3 | 609585 | 359.715 | −4.523019 | uc002ayu.1 | chr15: 75122696- | NA | NA | NA |
| | | | | | 75124136 | | | |
| CPN1 | 603103 | 695.242 | −5.603875 | uc001kql.2 | chr10: 101802065- | 1087.25 | −8.433296 | NA |
| | | | | | 101802183 | | | |
| CPNE1 | 604205 | NA | NA | NA | NA | 337.875 | −5.872445 | uc010zvj.2 |
| CPNE2 | 604206 | 606.873 | −11.461281 | uc002ekt.1 | chr16: 57180288- | NA | NA | NA |
| | | | | | 57181876 | | | |
| CPNE3 | 604207 | NA | NA | NA | NA | NA | NA | NA |
| CPNE4 | 604208 | 661.101 | −9.8835 | uc003eoj.3 | chr3: 131253577- | NA | NA | NA |
| | | | | | 131254038 | | | |
| CPPED1 | 615603 | NA | NA | NA | NA | 805.524 | −6.456126 | uc002dca.4 |
| CPSF2 | 606028 | NA | NA | NA | NA | 816.076 | −6.059901 | uc001yah.2 |
| CPSF7 | NA | 365.409 | −5.908002 | uc009ynp.2 | chr11: 61188420- | NA | NA | NA |
| | | | | | 61188801 | | | |
| CPT1B | 601987 | NA | NA | NA | NA | NA | NA | NA |
| CPTP | 615467 | 794.599 | −4.845648 | uc001aeo.3 | chr1: 1263144- | NA | NA | NA |
| | | | | | 1264276 | | | |
| CPZ | 603105 | NA | NA | NA | NA | 483.271 | −5.143917 | uc003glm.3 |
| CRABP1 | 180230 | NA | NA | NA | NA | NA | NA | NA |
| CRACR2A | 614178 | NA | NA | NA | NA | NA | NA | NA |
| CRCP | 606121 | NA | NA | NA | NA | 1448.07 | −13.092824 | uc003tus.3 |
| CREB3L2 | 608834 | NA | NA | NA | NA | NA | NA | NA |
| CREB5 | NA | 291.513 | −4.944841 | uc011jzr.2 | chr7: 28763989- | 221.677 | −3.09101 | uc003szo.3 |
| | | | | | 28764776 | | | |
| CREBRF | NA | 1746.71 | −7.57385 | uc003mcf.3 | chr5: 172518437- | 586.759 | −11.842591 | uc003mch.3 |
| | | | | | 172519333 | | | |
| CREM | 123812 | 1206 | −6.923832 | uc001ixz.3 | chr10: 35500327- | NA | NA | NA |
| | | | | | 35501886 | | | |
| CRHBP | 122559 | 657.115 | −7.318598 | uc010izx.3 | chr5: 76254739- | NA | NA | NA |
| | | | | | 76254847 | | | |
| CRIPAK | 610203 | NA | NA | NA | NA | NA | NA | NA |
| CRISP1 | 601193 | NA | NA | NA | NA | 1209.89 | −6.382111 | uc003ozw.2 |
| CRISPLD2 | 612434 | 1581.68 | −8.004311 | uc002fik.4 | chr16: 84872582- | 807.304 | −7.970899 | uc002fik.4 |
| | | | | | 84873626 | | | |
| CRK | 164762 | NA | NA | NA | NA | 787.286 | −6.122336 | uc002fsl.3 |
| CRKL | 602007 | 303.225 | −5.60498 | uc002ztf.2 | chr22: 21304134- | 279.288 | −7.858606 | uc002ztf.2 |
| | | | | | 21308037 | | | |
| CRLF3 | 614853 | 541.032 | −4.731745 | uc002hfr.4 | chr17: 29109702- | 377.806 | −5.024132 | uc002hfr.4 |
| | | | | | 29111204 | | | |
| CRLS1 | 608188 | NA | NA | NA | NA | 271.302 | −5.713495 | uc002wmn.4 |
| CRNKL1 | 610952 | 353.89 | −8.035045 | uc002wrs.3 | chr20: 20015012- | 919.675 | −9.479613 | uc002wrs.3 |
| | | | | | 20016838 | | | |
| CRNN | 611312 | NA | NA | NA | NA | NA | NA | NA |
| CRTAP | 605497 | NA | NA | NA | NA | NA | NA | NA |
| CRYAA | 123580 | NA | NA | NA | NA | NA | NA | NA |
| CRYBA4 | 123631 | 710.618 | −5.94855 | uc003acz.4 | chr22: 27026452- | NA | NA | NA |
| | | | | | 27026636 | | | |
| CRYGS | 123730 | NA | NA | NA | NA | 920.699 | −4.507734 | uc003fqe.3 |
| CSAD | 616569 | 534.851 | −4.355081 | uc021qyc.1 | chr12: 53551447- | NA | NA | NA |
| | | | | | 53552294 | | | |
| CSF1R | 164770 | NA | NA | NA | NA | NA | NA | NA |
| CSF2RA | 306250 | 324.06 | −4.84583 | uc004cpn.2 | chrX: 1428373- | NA | NA | NA |
| | | | | | 1428828 | | | |
| CSF3 | 138970 | NA | NA | NA | NA | NA | NA | NA |
| CSH2 | 118820 | 407.693 | −9.564075 | NA | NA | 570.379 | −5.15376 | uc002jci.3 |
| CSMD1 | 608397 | NA | NA | NA | NA | NA | NA | NA |
| CSMD3 | 608399 | NA | NA | NA | NA | 410.246 | −5.015578 | uc003yns.3 |
| CSN1S2BP | NA | NA | NA | NA | NA | NA | NA | NA |
| CSNK1D | 600864 | 434.373 | −6.920977 | uc002keh.3 | chr17: 80200537- | 252.025 | −4.988412 | uc002keh.3 |
| | | | | | 80202707 | | | |
| CSNK2B | 115441 | 538.04 | −3.005375 | uc021yuv.1 | chr6: 31636474- | 452.761 | −4.20991 | uc021yuv.1 |
| | | | | | 31636877 | | | |
| CSRNP2 | NA | NA | NA | NA | NA | NA | NA | NA |
| CST9L | 616536 | NA | NA | NA | NA | NA | NA | NA |
| CSTF2 | 300907 | 928.963 | −6.132082 | uc004egh.3 | chrX: 100095703- | 256.295 | −5.458538 | uc004egh.3 |
| | | | | | 100095923 | | | |
| CSTF2T | 611968 | NA | NA | NA | NA | NA | NA | NA |
| CSTF3 | 600367 | 623.846 | −6.591082 | uc001mui.3 | chr11: 33162977- | NA | NA | NA |
| | | | | | 33163125 | | | |
| CT45A4 | NA | NA | NA | NA | NA | NA | NA | NA |
| CT55 | NA | NA | NA | NA | NA | 853.13 | −5.099143 | uc004eyl.1 |
| CT62 | NA | NA | NA | NA | NA | 375.652 | −6.530172 | uc002ata.2 |
| CTAG2 | 300396 | NA | NA | NA | NA | 614.469 | −5.019963 | uc004fmi.2 |
| CTAGE5 | 602132 | NA | NA | NA | NA | 314.408 | −4.007171 | uc001wuy.4 |
| CTBS | 600873 | 1005.59 | −10.21535 | uc001dkc.3 | chr1: 85018804- | NA | NA | NA |
| | | | | | 85020681 | | | |
| CTCF | 604167 | 622.953 | −4.437291 | uc002etl.3 | chr16: 67671776- | NA | NA | NA |
| | | | | | 67673088 | | | |
| CTCFL | 607022 | 1304.88 | −8.684794 | uc010giw.1 | chr20: 56071021- | NA | NA | NA |
| | | | | | 56071305 | | | |
| CTDSPL2 | NA | NA | NA | NA | NA | NA | NA | NA |
| CTF1 | 600435 | NA | NA | NA | NA | NA | NA | NA |
| CTNNBL1 | 611537 | NA | NA | NA | NA | 963.359 | −4.396525 | uc021wdj.1 |
| CTNND2 | 604275 | 727.906 | −6.938236 | uc011cmx.1 | chr5: 10971952- | 683.687 | −8.035218 | uc011cmx.1 |
| | | | | | 10973564 | | | |
| CTPS1 | 123860 | NA | NA | NA | NA | 422.263 | −5.646279 | uc001cgk.4 |
| CTRL | 118888 | NA | NA | NA | NA | 533.89 | −4.052087 | uc002euw.3 |
| CTSK | 601105 | 526.844 | −6.070913 | uc001evp.2 | chr1: 150768684- | 207.373 | −5.076075 | uc001evp.2 |
| | | | | | 150769274 | | | |
| CTSO | 600550 | NA | NA | NA | NA | NA | NA | NA |
| CTSS | 116845 | NA | NA | NA | NA | 317.205 | −4.45596 | uc001evn.3 |
| CTSV | 603308 | NA | NA | NA | NA | 406.508 | −3.926191 | uc004awt.3 |
| CTTN | 164765 | 449.083 | −6.181621 | uc001opu.3 | chr11: 70282515- | 1714.88 | −10.593976 | uc001opv.4 |
| | | | | | 70282690 | | | |
| CTTNBP2 | 609772 | NA | NA | NA | NA | 945.06 | −10.114267 | uc003vjf.3 |
| CTXN2 | NA | 373.162 | −4.37522 | uc001zwm.1 | chr15: 48493744- | 1096.35 | −9.219889 | uc001zwm.1 |
| | | | | | 48495951 | | | |
| CUL2 | 603135 | 765.554 | −10.446254 | uc009xma.3 | chr10: 35297479- | 889.619 | −7.113834 | uc009xma.3 |
| | | | | | 35299238 | | | |
| CUL4B | 300304 | 581.91 | −4.155313 | uc010nqq.3 | chrX: 119658446- | NA | NA | NA |
| | | | | | 119660615 | | | |
| CUL5 | 601741 | NA | NA | NA | NA | 620.767 | −6.65374 | uc001pjv.3 |
| CWC15 | NA | NA | NA | NA | NA | 813.018 | −7.59694 | uc009ywl.1 |
| CWC22 | 615186 | NA | NA | NA | NA | NA | NA | NA |
| CX3CR1 | 601470 | 686.455 | −4.88608 | uc003cjl.3 | chr3: 39304985- | NA | NA | NA |
| | | | | | 39306932 | | | |
| CXADR | 602621 | 535.656 | −6.112243 | uc002yki.3 | chr21: 18938011- | 772.511 | −4.449546 | uc002ykl.3 |
| | | | | | 18942429 | | | |
| CXCL10 | 147310 | NA | NA | NA | NA | 736.732 | −7.221476 | uc003hjl.4 |
| CXCL13 | 605149 | NA | NA | NA | NA | 1041.29 | −10.827015 | uc003hkr.3 |
| CXCL16 | 605398 | NA | NA | NA | NA | 293.67 | −7.196354 | uc002fyr.4 |
| CXCL2 | 139110 | NA | NA | NA | NA | NA | NA | NA |
| CXCL8 | 146930 | NA | NA | NA | NA | NA | NA | NA |
| CXCL9 | 601704 | NA | NA | NA | NA | NA | NA | NA |
| CXCR5 | 601613 | NA | NA | NA | NA | 684.652 | −5.429545 | uc001pue.4 |
| CXCR6 | 605163 | NA | NA | NA | NA | NA | NA | NA |
| CXorf38 | NA | NA | NA | NA | NA | 1050.31 | −9.588863 | uc004dew.3 |
| CXorf40B | NA | 321.784 | −5.277387 | uc004fdy.3 | chrX: 149100415- | NA | NA | NA |
| | | | | | 149100761 | | | |
| CYB561D1 | NA | 327.936 | −5.109716 | uc010ovl.2 | chr1: 110038882- | NA | NA | NA |
| | | | | | 110043063 | | | |
| CYB5B | 611964 | 618.221 | −6.143686 | uc002exf.3 | chr16: 69482099- | NA | NA | NA |
| | | | | | 69484124 | | | |
| CYB5D1 | NA | NA | NA | NA | NA | 200.72 | −4.750343 | uc002gjb.4 |
| CYB5R2 | 608342 | NA | NA | NA | NA | 852.503 | −2.676455 | uc001mfm.3 |
| CYB5R4 | 608343 | NA | NA | NA | NA | 704.169 | −6.295885 | uc003pkf.3 |
| CYFIP1 | 606322 | NA | NA | NA | NA | NA | NA | NA |
| CYLC2 | 604035 | NA | NA | NA | NA | 775.97 | −8.25712 | uc004bbs.2 |
| CYLD | 605018 | 1736.44 | 0.437262 | uc002ego.3 | chr16: 50818367- | NA | NA | NA |
| | | | | | 50820674 | | | |
| CYP19A1 | 107910 | NA | NA | NA | NA | 620.377 | −7.528639 | uc001zyz.4 |
| CYP1A1 | 108330 | NA | NA | NA | NA | NA | NA | NA |
| CYP1A2 | 124060 | NA | NA | NA | NA | 767.437 | −5.161176 | uc002ayr.1 |
| CYP1B1 | 601771 | NA | NA | NA | NA | NA | NA | NA |
| CYP20A1 | NA | NA | NA | NA | NA | NA | NA | NA |
| CYP21A2 | 613815 | NA | NA | NA | NA | 294.213 | −4.823852 | uc003nze.2 |
| CYP24A1 | 126065 | NA | NA | NA | NA | 436.611 | −4.032327 | uc002xwu.1 |
| CYP27B1 | 609506 | NA | NA | NA | NA | NA | NA | NA |
| CYP2C9 | 601130 | 633.449 | −7.529759 | uc001kjz.3 | chr10: 96702107- | 417.617 | −4.060501 | uc001kka.4 |
| | | | | | 96703022 | | | |
| CYP2E1 | 124040 | NA | NA | NA | NA | 373.895 | −8.472378 | uc001lnj.1 |
| CYP3A5 | 605325 | NA | NA | NA | NA | 283.268 | −5.622688 | uc003urt.3 |
| CYP46A1 | 604087 | NA | NA | NA | NA | 515.48 | −7.533667 | uc001ygn.1 |
| CYP4F12 | 611485 | 629.633 | −1.929685 | uc010xoo.2 | chr19: 15791495- | 447.481 | −7.8522 | uc010xoo.2 |
| | | | | | 15791754 | | | |
| CYP4F3 | 601270 | NA | NA | NA | NA | NA | NA | NA |
| CYP4F8 | 611545 | NA | NA | NA | NA | 685.675 | −7.854855 | uc010xoi.1 |
| CYP4V2 | 608614 | NA | NA | NA | NA | 490.319 | −5.090941 | uc003iyw.4 |
| CYP4X1 | 614999 | NA | NA | NA | NA | 447.33 | −4.106452 | uc001cqr.3 |
| CYP4Z1 | NA | NA | NA | NA | NA | 295.974 | −5.233557 | uc001cqu.1 |
| CYP51A1 | 601637 | NA | NA | NA | NA | 673.092 | −10.440726 | uc003ulm.4 |
| CYSLTR2 | 605666 | NA | NA | NA | NA | 688.469 | −8.972358 | uc001vck.2 |
| CYTH1 | 182115 | 277.993 | −4.63134 | uc002jvw.3 | chr17: 76670130- | NA | NA | NA |
| | | | | | 76672172 | | | |
| CYTH2 | 602488 | 1260.18 | −6.391384 | uc010xzr.1 | chr19: 48975873- | NA | NA | NA |
| | | | | | 48976236 | | | |
| CYTIP | 604448 | 556.997 | −7.722992 | uc002tzj.1 | chr2: 158271131- | NA | NA | NA |
| | | | | | 158272188 | | | |
| CYYR1 | 616020 | NA | NA | NA | NA | NA | NA | NA |
| DACT1 | 607861 | NA | NA | NA | NA | NA | NA | NA |
| DACT2 | 608966 | NA | NA | NA | NA | NA | NA | NA |
| DAND5 | 609068 | NA | NA | NA | NA | 593.023 | −4.067544 | uc010dyz.1 |
| DAPK1 | 600831 | 620.783 | −6.749656 | uc004apc.3 | chr9: 90322280- | NA | NA | NA |
| | | | | | 90323549 | | | |
| DAPP1 | 605768 | NA | NA | NA | NA | 286.066 | −5.758828 | uc003hvf.4 |
| DARS | 603084 | 325.411 | −4.024679 | uc002tux.1 | chr2: 136664254- | NA | NA | NA |
| | | | | | 136664885 | | | |
| DAZL | 601486 | NA | NA | NA | NA | NA | NA | NA |
| DBF4 | 604281 | 903.142 | −8.625398 | uc003ujf.1 | chr7: 87537479- | NA | NA | NA |
| | | | | | 87538856 | | | |
| DBI | 125950 | NA | NA | NA | NA | NA | NA | NA |
| DBR1 | 607024 | 1156.92 | −9.485672 | uc003ert.3 | chr3: 137879830- | 1323.06 | −14.673025 | uc003ert.3 |
| | | | | | 137880730 | | | |
| DCAF12 | NA | NA | NA | NA | NA | NA | NA | NA |
| DCAF12L2 | NA | NA | NA | NA | NA | 1392.34 | −10.768565 | uc004euk.2 |
| DCAF13 | 616196 | 1408.61 | −13.240869 | uc003yln.3 | chr8: 104455111- | NA | NA | NA |
| | | | | | 104455680 | | | |
| DCAF16 | NA | NA | NA | NA | NA | 999.771 | −6.921306 | uc003gpn.3 |
| DCAF5 | 603812 | 1072.51 | −11.185222 | uc001xkr.4 | chr14: 69555921- | NA | NA | NA |
| | | | | | 69557771 | | | |
| DCAF6 | 610494 | NA | NA | NA | NA | NA | NA | NA |
| DCAKD | NA | 645.574 | −7.896472 | uc002ihx.2 | chr17: 43100706- | NA | NA | NA |
| | | | | | 43101800 | | | |
| DCBLD1 | NA | NA | NA | NA | NA | 452.656 | −13.535796 | uc003pxr.1 |
| DCBLD2 | 608698 | NA | NA | NA | NA | NA | NA | NA |
| DCK | 125450 | NA | NA | NA | NA | 616.599 | −9.061247 | uc003hfx.3 |
| DCLK1 | 604742 | NA | NA | NA | NA | 265.232 | −5.089665 | uc001uve.4 |
| DCN | 125255 | NA | NA | NA | NA | NA | NA | NA |
| DCP1B | 609843 | 348.257 | −5.97131 | uc001qjx.1 | chr12: 2055214- | NA | NA | NA |
| | | | | | 2055371 | | | |
| DCT | 191275 | NA | NA | NA | NA | 858.464 | −7.342876 | uc001vlv.4 |
| DCTD | 607638 | NA | NA | NA | NA | NA | NA | NA |
| DCTPP1 | 615840 | NA | NA | NA | NA | NA | NA | NA |
| DCUN1D1 | 605905 | NA | NA | NA | NA | NA | NA | NA |
| DCX | 300121 | 1460.17 | −15.900962 | uc004epd.3 | chrX: 110537007- | 1811.46 | −14.881737 | uc004epd.3 |
| | | | | | 110544914 | | | |
| DDAH1 | 604743 | 1761.87 | −12.814762 | uc010osb.2 | chr1: 85784168- | NA | NA | NA |
| | | | | | 85787134 | | | |
| DDHD1 | 614603 | 970.37 | −9.085704 | uc001xag.3 | chr14: 53503458- | NA | NA | NA |
| | | | | | 53513485 | | | |
| DDHD2 | 615003 | 275.594 | −6.287877 | uc003xla.2 | chr8: 38095790- | NA | NA | NA |
| | | | | | 38096610 | | | |
| DDI2 | NA | 573.172 | −8.429707 | uc001awx.2 | chr1: 15986332- | NA | NA | NA |
| | | | | | 15987552 | | | |
| DDN | 610588 | 618.798 | −5.490174 | uc001rsv.1 | chr12: 49388933- | 1178.69 | −6.411042 | uc001rsv.1 |
| | | | | | 49390522 | | | |
| DDOST | 602202 | 448.293 | −7.264193 | uc001bdo.1 | chr1: 20978260- | 870.689 | −9.553412 | uc001bdo.1 |
| | | | | | 20978871 | | | |
| DDRGK1 | 616177 | 444.45 | −5.509458 | uc002wic.3 | chr20: 3171012- | 528.769 | −7.006966 | uc002wic.3 |
| | | | | | 3171298 | | | |
| DDT | 602750 | NA | NA | NA | NA | NA | NA | NA |
| DDX17 | 608469 | 1784.67 | −13.445906 | uc003avw.4 | chr22: 38879443- | NA | NA | NA |
| | | | | | 38881939 | | | |
| DDX18 | 606355 | NA | NA | NA | NA | NA | NA | NA |
| DDX31 | 616533 | 829.837 | −6.165336 | uc004cbq.1 | chr9: 135469676- | NA | NA | NA |
| | | | | | 135470252 | | | |
| DDX3Y | 400010 | NA | NA | NA | NA | 1057.01 | −5.569501 | uc010nwv.1 |
| DDX4 | 605281 | 629.852 | −7.595238 | uc003jqg.4 | chr5: 55112369- | NA | NA | NA |
| | | | | | 55112974 | | | |
| DDX47 | 615428 | NA | NA | NA | NA | 655.096 | −7.103075 | uc001rav.3 |
| DDX5 | 180630 | 1335.83 | −13.263429 | uc010deh.2 | chr17: 62494374- | NA | NA | NA |
| | | | | | 62496040 | | | |
| DDX52 | 612500 | 548.717 | −7.206606 | uc002hoh.2 | chr17: 35972363- | NA | NA | NA |
| | | | | | 35974340 | | | |
| DDX55 | NA | NA | NA | NA | NA | 975.709 | −6.450349 | uc001ufh.3 |
| DDX59 | 615464 | 781.681 | −8.136568 | uc009wzk.3 | chr1: 200613165- | 562.416 | −8.596412 | uc010ppl.1 |
| | | | | | 200613381 | | | |
| DDX6 | 600326 | 1099.42 | −6.227878 | uc001pua.2 | chr11: 118618473- | NA | NA | NA |
| | | | | | 118622806 | | | |
| DECR1 | 222745 | NA | NA | NA | NA | 773.017 | −8.16059 | uc003yek.1 |
| DEFA6 | 600471 | 675.215 | −8.389447 | uc003wqt.3 | chr8: 6782216- | NA | NA | NA |
| | | | | | 6782339 | | | |
| DEFB129 | NA | NA | NA | NA | NA | 572.708 | −4.466028 | uc002wda.3 |
| DEGS2 | 610862 | NA | NA | NA | NA | NA | NA | NA |
| DENND1B | 613292 | NA | NA | NA | NA | NA | NA | NA |
| DENND2C | NA | NA | NA | NA | NA | NA | NA | NA |
| DENND4A | 600382 | 965.819 | −4.343313 | uc002aph.3 | chr15: 65952957- | 1521.3 | −13.657735 | uc010ujj.1 |
| | | | | | 65954188 | | | |
| DENND4C | NA | NA | NA | NA | NA | NA | NA | NA |
| DENND5B | NA | 721.565 | −4.37695 | uc001rkj.3 | chr12: 31585311- | NA | NA | NA |
| | | | | | 31586043 | | | |
| DEPDC1B | 616073 | NA | NA | NA | NA | NA | NA | NA |
| DEPDC5 | 614191 | NA | NA | NA | NA | 548.903 | −4.874114 | uc011als.2 |
| DEPDC7 | 612294 | 527.57 | −6.369407 | uc010reg.1 | chr11: 33047705- | NA | NA | NA |
| | | | | | 33048263 | | | |
| DERA | NA | NA | NA | NA | NA | NA | NA | NA |
| DERL2 | 610304 | NA | NA | NA | NA | 1329.6 | −8.25838 | uc002gcc.1 |
| DFFA | 601882 | 1008.9 | −7.721187 | uc001arj.3 | chr1: 10520603- | 444.792 | −5.303749 | uc001arj.3 |
| | | | | | 10521546 | | | |
| DFFB | 601883 | NA | NA | NA | NA | 1659.64 | −16.558377 | uc001ala.1 |
| DGAT2 | 606983 | NA | NA | NA | NA | NA | NA | NA |
| DQCR6 | 601279 | 594.478 | −5.477706 | uc002zoh.4 | chr22: 18899203- | NA | NA | NA |
| | | | | | 18899601 | | | |
| DGKB | 604070 | 274.583 | −8.230584 | uc003ss2.3 | chr7: 14184674- | NA | NA | NA |
| | | | | | 14188755 | | | |
| DGKH | 604071 | 864.705 | −4.577431 | uc010tfh.2 | chr13: 42803288- | 894.565 | −11.205163 | uc010tfh.2 |
| | | | | | 42803891 | | | |
| DHFRL1 | 616588 | 982.962 | −8.429723 | uc003dri.3 | chr3: 93776766- | NA | NA | NA |
| | | | | | 93779791 | | | |
| DHPS | 600944 | 582.911 | −5.636385 | uc002mug.2 | chr19: 12786531- | NA | NA | NA |
| | | | | | 12786651 | | | |
| DHRS1 | 610410 | 718.851 | −4.630178 | uc001woj.2 | chr14: 24759804- | NA | NA | NA |
| | | | | | 24760064 | | | |
| DHRS11 | 616159 | NA | NA | NA | NA | 894.039 | −8.637033 | uc002hnd.3 |
| DHRS7 | 612833 | NA | NA | NA | NA | 244.111 | −3.650219 | uc001xes.3 |
| DHRS7B | 616160 | NA | NA | NA | NA | 792.518 | −4.47778 | uc002gyo.3 |
| DHX30 | 616423 | 967.982 | −5.266378 | uc003crs.2 | chr3: 47883246- | NA | NA | NA |
| | | | | | 47884169 | | | |
| DHX32 | 607960 | NA | NA | NA | NA | 750.456 | −5.650058 | uc001lje.1 |
| DHX33 | 614405 | 750.894 | −5.908518 | uc002gb2.3 | chr17: 5344232- | NA | NA | NA |
| | | | | | 5347524 | | | |
| DHX35 | NA | 448.165 | −7.534375 | uc002xjh.3 | chr20: 37667187- | NA | NA | NA |
| | | | | | 37668366 | | | |
| DHX40 | 607570 | 696.753 | −6.13796 | uc002ixn.2 | chr17: 57684534- | NA | NA | NA |
| | | | | | 57685713 | | | |
| DHX57 | NA | NA | NA | NA | NA | NA | NA | NA |
| DHX9 | 603115 | NA | NA | NA | NA | 1201.48 | −11.775117 | uc001gpr.3 |
| DICER1 | 606241 | 1661.91 | −11.931692 | uc010avh.1 | chr14: 95552565- | NA | NA | NA |
| | | | | | 95556834 | | | |
| DIEXF | NA | 221.794 | −7.613252 | uc001hhr.2 | chr1: 210024793- | NA | NA | NA |
| | | | | | 210030910 | | | |
| DIMT1 | 612499 | 1127.43 | −8.616824 | uc003jta.3 | chr5: 61684351- | NA | NA | NA |
| | | | | | 61684836 | | | |
| DIO1 | 147892 | NA | NA | NA | NA | NA | NA | NA |
| DIO2 | 601413 | NA | NA | NA | NA | 212.292 | −6.632222 | uc001xut.3 |
| DIP2A | 607711 | 1003.03 | −0.243268 | uc002zjl.3 | chr21: 47965920- | 685.863 | −6.582904 | uc002zjm.3 |
| | | | | | 47966219 | | | |
| DIP2C | 611380 | 916.377 | −3.202008 | uc001ifp.3 | chr10: 320130- | 445.25 | −7.083457 | uc001ifp.3 |
| | | | | | 323264 | | | |
| DIRAS3 | 605193 | NA | NA | NA | NA | 1217.86 | −9.92879 | uc001ded.3 |
| DIRC1 | 606423 | NA | NA | NA | NA | 1355.96 | −11.237693 | uc002uqi.1 |
| DIS3L | 614183 | 473.948 | −10.463499 | uc002app.3 | chr15: 66625651- | NA | NA | NA |
| | | | | | 66626236 | | | |
| DIXDC1 | 610493 | 357.58 | −4.696435 | uc001pmk.3 | chr11: 111845712- | NA | NA | NA |
| | | | | | 111846719 | | | |
| DKFZP434K028 | NA | NA | NA | NA | NA | NA | NA | NA |
| DLC1 | 604258 | 229.482 | −6.298196 | uc011kxx.1 | chr8: 12940872- | NA | NA | NA |
| | | | | | 12943319 | | | |
| DLD | 238331 | 496.813 | −6.195754 | uc003vet.3 | chr7: 107559705- | 548.171 | −5.375663 | uc003vet.3 |
| | | | | | 107561643 | | | |
| DLEU7 | NA | NA | NA | NA | NA | NA | NA | NA |
| DLG1 | 601014 | 458.282 | −6.156304 | uc011bub.2 | chr3: 196769431- | NA | NA | NA |
| | | | | | 196771493 | | | |
| DLG2 | 603583 | 851.421 | −10.890656 | uc010rsw.1 | chr11: 83166056- | 1583.36 | −8.320321 | uc010rsw.1 |
| | | | | | 83170860 | | | |
| DLG4 | 602887 | 540.129 | −6.993796 | uc002geu.3 | chr17: 7106037- | NA | NA | NA |
| | | | | | 7106108 | | | |
| DLG5 | 604090 | 370.062 | −3.921482 | uc001jzi.3 | chr10: 79550549- | 349.491 | −5.246477 | uc001jzl.4 |
| | | | | | 79552197 | | | |
| DLGAP5 | NA | NA | NA | NA | NA | NA | NA | NA |
| DLK1 | 176290 | NA | NA | NA | NA | NA | NA | NA |
| DLK2 | NA | NA | NA | NA | NA | 447.075 | −7.563602 | uc003ova.3 |
| DLX2 | 126255 | NA | NA | NA | NA | NA | NA | NA |
| DLX5 | 600028 | NA | NA | NA | NA | 339.212 | −4.336061 | uc003uon.3 |
| DMAP1 | 605077 | NA | NA | NA | NA | 587.919 | −4.06949 | uc010oks.1 |
| DMBT1 | 601969 | 556.926 | −4.585484 | uc001lgk.1 | chr10: 124402915- | NA | NA | NA |
| | | | | | 124403252 | | | |
| DMD | 300377 | 583.476 | −2.809975 | Uc004dcm.1 | chrX: 31137345- | 637.37 | −8.341886 | uc004dco.1 |
| | | | | | 31140035 | | | |
| DMPK | 605377 | NA | NA | NA | NA | 1056.69 | −7.514724 | uc021uwb.1 |
| DMRT1 | 602424 | 673.593 | −6.418252 | uc003zgv.3 | chr9: 968140- | NA | NA | NA |
| | | | | | 969090 | | | |
| DMRTA1 | 614803 | NA | NA | NA | NA | NA | NA | NA |
| DMRTA2 | 614804 | NA | NA | NA | NA | 319.81 | −5.207294 | uc010ona.2 |
| DMRTB1 | 614805 | NA | NA | NA | NA | 505.055 | −6.51192 | uc001cvq.1 |
| DMXL1 | 605671 | 846.036 | −6.421941 | uc003ksd.2 | chr5: 118582915- | 408.936 | −7.84824 | uc003ksd.2 |
| | | | | | 118584822 | | | |
| DMXL2 | 612186 | 622.682 | −5.425405 | uc002abd.3 | chr15: 51739921- | 824.368 | −11.650011 | uc002abd.3 |
| | | | | | 51741180 | | | |
| DNER | 607299 | 334.361 | −4.46242 | uc002vpv.3 | chr2: 230222345- | NA | NA | NA |
| | | | | | 230223255 | | | |
| DNHD1 | NA | NA | NA | NA | NA | 236.858 | −4.821863 | uc001mdp.3 |
| DNM2 | 602378 | NA | NA | NA | NA | NA | NA | NA |
| DNM3 | 611445 | 258.15 | −6.549519 | NA | NA | NA | NA | NA |
| DNMT3A | 602769 | 261.844 | −4.084102 | uc002rgf.3 | chr2: 25504321- | NA | NA | NA |
| | | | | | 25505256 | | | |
| DNTT | 187410 | 901.814 | −10.272594 | uc001kmf.3 | chr10: 98097982- | 622.571 | −4.515916 | uc001kmf.3 |
| | | | | | 98098321 | | | |
| DNTTIP1 | 611388 | NA | NA | NA | NA | 337.666 | −4.141338 | uc002xpk.3 |
| DNTTIP2 | 611199 | 870.64 | −9.984598 | uc001dqf.3 | chr1: 94335014- | 962.589 | −7.358886 | uc001dqf.3 |
| | | | | | 94335406 | | | |
| DOCK10 | 611518 | NA | NA | NA | NA | NA | NA | NA |
| DOCK4 | 607679 | NA | NA | NA | NA | NA | NA | NA |
| DOCK7 | 615730 | NA | NA | NA | NA | 583.283 | −6.684065 | uc001dam.3 |
| DOK2 | 604997 | NA | NA | NA | NA | NA | NA | NA |
| DOK7 | 610285 | 365.471 | −6.737641 | uc003ghe.3 | chr4: 3494493- | NA | NA | NA |
| | | | | | 3496209 | | | |
| DOPEY2 | 604803 | NA | NA | NA | NA | 555.88 | −7.142553 | uc002yvh.3 |
| DPAGT1 | 191350 | 945.55 | −14.44732 | uc001pvm.1 | chr11: 118967221- | NA | NA | NA |
| | | | | | 118969108 | | | |
| DPF3 | 601672 | NA | NA | NA | NA | 1373.09 | −17.250253 | uc010ari.1 |
| DPH6 | NA | NA | NA | NA | NA | NA | NA | NA |
| DPM1 | 603503 | NA | NA | NA | NA | 933.576 | −9.581747 | uc002xvw.1 |
| DPP8 | 606819 | NA | NA | NA | NA | 530.771 | −4.482077 | uc010bhi.3 |
| DPPA3 | 608408 | 2143.7 | −19.858836 | uc001qtf.3 | chr12: 7869674- | NA | NA | NA |
| | | | | | 7870152 | | | |
| DPT | 125597 | 269.678 | −8.015586 | uc001gfp.3 | chr1: 168664695- | NA | NA | NA |
| | | | | | 168665786 | | | |
| DPY19L1 | 613892 | 589.57 | −10.378934 | uc003tem.4 | chr7: 34968493- | 651.525 | −8.950194 | uc003tem.4 |
| | | | | | 34971184 | | | |
| DPY19L2 | 613893 | NA | NA | NA | NA | 506.986 | −8.411004 | uc010sso.1 |
| DPY19L2P2 | NA | NA | NA | NA | NA | 636.551 | −5.455464 | uc003vbh.4 |
| DPY19L4 | 613895 | 1334.18 | −7.207728 | uc003ygx.2 | chr8: 95802139- | NA | NA | NA |
| | | | | | 95806076 | | | |
| DR1 | 601482 | NA | NA | NA | NA | 533.937 | −5.597409 | uc001dpu.3 |
| DROSHA | 608828 | 1048.41 | −10.990914 | uc003jhg.2 | chr5: 31400602- | NA | NA | NA |
| | | | | | 31401538 | | | |
| DSC3 | 600271 | NA | NA | NA | NA | 695.561 | −4.84583 | uc002kwi.4 |
| DSCAM | 602523 | NA | NA | NA | NA | 296.666 | −7.140517 | uc002yyq.1 |
| DSCR3 | 605298 | 868.171 | −12.762657 | uc002ywf.1 | chr21: 38595726- | 687.193 | −6.56759 | uc002ywf.1 |
| | | | | | 38597844 | | | |
| DSE | 605942 | NA | NA | NA | NA | NA | NA | NA |
| DSG1 | 125670 | NA | NA | NA | NA | NA | NA | NA |
| DSG3 | 169615 | NA | NA | NA | NA | 535.982 | −5.735543 | uc002kws.3 |
| DSG4 | 607892 | NA | NA | NA | NA | 579.529 | −5.685449 | uc002kwq.2 |
| DSP | 125647 | NA | NA | NA | NA | NA | NA | NA |
| DST | 113810 | 581.633 | −6.136382 | uc003pcv.4 | chr6: 56322785- | 1545.4 | −7.780119 | uc003pda.4 |
| | | | | | 56323802 | | | |
| DSTN | 609114 | 725.091 | −7.72674 | uc002wpq.3 | chr20: 17587792- | NA | NA | NA |
| | | | | | 17588652 | | | |
| DTD2 | NA | NA | NA | NA | NA | 842.159 | −7.795574 | uc001wrj.3 |
| DTNBP1 | 607145 | 319.292 | −6.45253 | uc003nbl.3 | chr6: 15523032- | NA | NA | NA |
| | | | | | 15523205 | | | |
| DTWD1 | NA | 1730.31 | −15.525711 | uc001zxq.3 | chr15: 49935776- | NA | NA | NA |
| | | | | | 49937333 | | | |
| DTX2 | 613141 | NA | NA | NA | NA | 1049.93 | −1.852145 | uc003uff.4 |
| DUSP10 | 608867 | NA | NA | NA | NA | 1203.66 | −10.477416 | uc001hmx.2 |
| DUSP12 | 604835 | NA | NA | NA | NA | NA | NA | NA |
| DUSP16 | 607175 | NA | NA | NA | NA | NA | NA | NA |
| DUSP18 | 611446 | NA | NA | NA | NA | NA | NA | NA |
| DUSP19 | 611437 | NA | NA | NA | NA | 1072.35 | −8.925386 | uc002upd.3 |
| DUSP22 | NA | 269.192 | −5.511994 | uc011dhn.1 | chr6: 348952- | NA | NA | NA |
| | | | | | 350227 | | | |
| DUSP28 | NA | 592.711 | −6.792756 | uc002vzg.3 | chr2: 241503091- | 217.456 | −4.636951 | uc002vzg.3 |
| | | | | | 241503431 | | | |
| DUSP5 | 603069 | 531.262 | −7.94301 | uc001kzd.3 | chr10: 112270185- | NA | NA | NA |
| | | | | | 112271302 | | | |
| DUSP5P1 | NA | NA | NA | NA | NA | 241.967 | −5.520958 | uc001htd.2 |
| DUSP6 | 602748 | NA | NA | NA | NA | 811.75 | −5.153969 | uc001tay.3 |
| DUSP8 | 602038 | NA | NA | NA | NA | 483.622 | −6.479288 | uc001lts.2 |
| DUT | 601266 | 1062.54 | −6.736045 | uc001zws.3 | chr15: 48634276- | NA | NA | NA |
| | | | | | 48635570 | | | |
| DUXA | 611168 | 1233.3 | −7.633809 | uc002qoa.1 | chr19: 57663094- | NA | NA | NA |
| | | | | | 57665779 | | | |
| DVL3 | 601368 | NA | NA | NA | NA | 752.027 | −7.820199 | uc003fms.3 |
| DYM | 607461 | NA | NA | NA | NA | 1204.44 | −6.49732 | uc002ldi.1 |
| DYNC1H1 | 600112 | 382.123 | −5.840876 | uc001yks.2 | chr14: 102516901- | NA | NA | NA |
| | | | | | 102517135 | | | |
| DYNC1LI1 | 615890 | 606.211 | −4.658034 | uc003cfb.4 | chr3: 32567463- | NA | NA | NA |
| | | | | | 32568290 | | | |
| DYNC2LI1 | NA | 567.962 | −6.720737 | uc002rth.3 | chr2: 44021882- | 298.427 | −16.563237 | uc002rti.3 |
| | | | | | 44022316 | | | |
| DYNLT1 | 601554 | 1081.04 | −5.254383 | uc003qrn.2 | chr6: 159057507- | NA | NA | NA |
| | | | | | 159057858 | | | |
| DYRK1A | 600855 | NA | NA | NA | NA | 611.055 | −7.520362 | uc002ywi.3 |
| DYRK3 | 603497 | NA | NA | NA | NA | 1033.86 | −6.287354 | uc001hei.3 |
| DYRK4 | 609181 | NA | NA | NA | NA | NA | NA | NA |
| DYTN | NA | NA | NA | NA | NA | NA | NA | NA |
| DZIP1 | 608671 | 567.604 | −5.603994 | uc001vmj.3 | chr13: 96230456- | NA | NA | NA |
| | | | | | 96234487 | | | |
| DZIP3 | 608672 | 561.452 | −7.413131 | uc003dxf.1 | chr3: 108409745- | 479.751 | −5.546902 | uc003dxf.1 |
| | | | | | 108410234 | | | |
| E2F5 | 600967 | 286.263 | −5.248158 | uc003ycz.4 | chr8: 86126098- | NA | NA | NA |
| | | | | | 86126753 | | | |
| EAF1 | 608315 | NA | NA | NA | NA | 567.722 | −6.762516 | uc003bzu.3 |
| EAPP | 609486 | 572.769 | −10.282138 | uc001wsd.1 | chr14: 34985135- | NA | NA | NA |
| | | | | | 34985515 | | | |
| EARS2 | 612799 | 617.831 | −8.366133 | uc002dlt.4 | chr16: 23533334- | 299.78 | −3.881119 | uc002dlt.4 |
| | | | | | 23535691 | | | |
| EBF2 | 609934 | 459.333 | −6.416353 | uc003xet.2 | chr8: 25888966- | NA | NA | NA |
| | | | | | 25890596 | | | |
| EBI3 | 605816 | NA | NA | NA | NA | 289.261 | −4.079749 | uc002lzu.3 |
| EBLN1 | 613249 | 540.054 | −6.407206 | uc021pob.1 | chr10: 22497743- | NA | NA | NA |
| | | | | | 22497811 | | | |
| EBPL | NA | 658.264 | −5.428213 | uc001vdi.3 | chr13: 50234810- | NA | NA | NA |
| | | | | | 50235344 | | | |
| ECM2 | 603479 | NA | NA | NA | NA | NA | NA | NA |
| ECSIT | 608388 | NA | NA | NA | NA | 582.499 | −6.357429 | uc010dyc.2 |
| EDA2R | 300276 | NA | NA | NA | NA | 681.641 | −4.575303 | uc004dwt.2 |
| EDC3 | 609842 | 572.35 | −6.037678 | uc002aym.3 | chr15: 74922899- | NA | NA | NA |
| | | | | | 74924952 | | | |
| EDC4 | 606030 | NA | NA | NA | NA | NA | NA | NA |
| EDN1 | 131240 | NA | NA | NA | NA | 545.625 | −8.79093 | uc010jpb.3 |
| EDN3 | 131242 | 468.869 | −5.108479 | uc002yap.3 | chr20: 57899751- | NA | NA | NA |
| | | | | | 57901047 | | | |
| EDNRB | 131244 | 813.132 | −5.433751 | uc001vko.2 | chr13: 78469616- | 786.956 | −8.545551 | uc001vko.2 |
| | | | | | 78472334 | | | |
| EDRF1 | NA | 1318.5 | −14.619822 | uc001lio.1 | chr10: 127452042- | 1566.13 | −16.658293 | uc001lin.3 |
| | | | | | 127452712 | | | |
| EFCAB11 | NA | 977.138 | −7.686403 | uc001xxw.2 | chr14: 90389670- | 563.822 | −5.1118 | uc001xxs.3 |
| | | | | | 90391002 | | | |
| EFCAB14 | NA | NA | NA | NA | NA | 628.518 | −5.787714 | uc010omj.2 |
| EFCAB3 | NA | NA | NA | NA | NA | 1840.12 | −19.237885 | uc010wpc.2 |
| EFCAB5 | NA | NA | NA | NA | NA | NA | NA | NA |
| EFCAB7 | NA | 221.099 | −7.898175 | uc001dbf.3 | chr1: 64038188- | NA | NA | NA |
| | | | | | 64038364 | | | |
| EFHB | NA | 377.909 | −4.501018 | uc003cbl.4 | chr3: 19920966- | NA | NA | NA |
| | | | | | 19921122 | | | |
| EFHC2 | 300817 | 846.797 | −9.936115 | uc022bvg.1 | chrX: 44007128- | 808.194 | −9.697979 | uc022bvg.1 |
| | | | | | 44008040 | | | |
| EFNB1 | 300035 | NA | NA | NA | NA | 930.437 | −9.84382 | uc004dxd.4 |
| EFNB2 | 600527 | 334.159 | −4.391684 | uc001vqi.3 | chr13: 107142079- | NA | NA | NA |
| | | | | | 107145387 | | | |
| EFNB3 | 602297 | NA | NA | NA | NA | 521.23 | −4.254039 | uc002gis.3 |
| EFTUD1 | NA | NA | NA | NA | NA | 1023.07 | −8.71403 | uc002bgt.1 |
| EFTUD2 | 603892 | NA | NA | NA | NA | NA | NA | NA |
| EGFEM1P | NA | 341.259 | −5.715728 | uc003ffh.4 | chr3: 168547033- | NA | NA | NA |
| | | | | | 168548374 | | | |
| EGFL6 | 300239 | 1697.5 | −12.053467 | uc004cvi.3 | chrX: 13651215- | NA | NA | NA |
| | | | | | 13651694 | | | |
| EGLN1 | 606425 | NA | NA | NA | NA | 511.562 | −7.43534 | uc001huu.3 |
| EHBP1 | 609922 | NA | NA | NA | NA | 896.401 | −10.495377 | uc010fcq.1 |
| EHD3 | 605891 | NA | NA | NA | NA | NA | NA | NA |
| EHD4 | 605892 | 586.436 | −7.230777 | uc001zot.3 | chr15: 42191639- | NA | NA | NA |
| | | | | | 42192842 | | | |
| EID1 | 605894 | 739.312 | −4.803936 | uc001zxc.1 | chr15: 49170938- | NA | NA | NA |
| | | | | | 49172380 | | | |
| EID2 | 609773 | NA | NA | NA | NA | 442.73 | −6.036513 | uc002oma.3 |
| EID3 | 612986 | 1101.04 | −8.130276 | uc001tkw.3 | chr12: 104698715- | NA | NA | NA |
| | | | | | 104698982 | | | |
| EIF1AD | NA | 542.606 | −4.818991 | uc001ogm.2 | chr11: 65764016- | NA | NA | NA |
| | | | | | 65766069 | | | |
| EIF1AX | 300186 | 565.952 | −7.934214 | uc004czt.3 | chrX: 20142636- | 261.596 | −5.04028 | uc004czt.3 |
| | | | | | 20146423 | | | |
| EIF1AY | 400014 | NA | NA | NA | NA | 614.58 | −6.923764 | uc004fuk.3 |
| EIF2AK4 | 609280 | 696.407 | −5.257692 | uc001zkl.3 | chr15: 40265984- | 579.136 | −7.028584 | uc001zkl.3 |
| | | | | | 40267625 | | | |
| EIF2B4 | 606687 | NA | NA | NA | NA | NA | NA | NA |
| EIF2D | 613709 | 480.947 | −4.615429 | uc001heh.2 | chr1: 206764974- | 883.254 | −6.328861 | uc001heh.2 |
| | | | | | 206765106 | | | |
| EIF2S1 | 603907 | NA | NA | NA | NA | NA | NA | NA |
| EIF2S2 | 603908 | NA | NA | NA | NA | 861.653 | −5.97446 | uc002xaf.3 |
| EIF2S3 | 300161 | NA | NA | NA | NA | 895.451 | −5.449679 | uc004dbc.3 |
| EIF3G | 603913 | 833.222 | −6.04034 | uc002mnd.3 | chr19: 10225690- | NA | NA | NA |
| | | | | | 10225789 | | | |
| EIF3H | 603912 | NA | NA | NA | NA | 1521.34 | −13.976626 | uc011lhz.1 |
| EIF3IP1 | NA | 575.943 | −7.129522 | uc003vfp.1 | chr7: 109599284- | NA | NA | NA |
| | | | | | 109599680 | | | |
| EIF3J | 603910 | 896.946 | −7.168892 | uc001ztv.3 | chr15: 44853348- | 704.344 | −8.175658 | uc001ztv.3 |
| | | | | | 44855001 | | | |
| EIF3L | NA | NA | NA | NA | NA | NA | NA | NA |
| EIF4A2 | 601102 | NA | NA | NA | NA | NA | NA | NA |
| EIF4A3 | 608546 | NA | NA | NA | NA | 1090.5 | −6.630955 | uc010wuc.2 |
| EIF4B | 603928 | 550.648 | −4.497664 | uc001sbh.4 | chr12: 53434008- | NA | NA | NA |
| | | | | | 53435993 | | | |
| EIF4E | 133440 | 734.043 | −4.904694 | uc011cec.1 | chr4: 99807502- | NA | NA | NA |
| | | | | | 99808217 | | | |
| EIF4E2 | 605895 | NA | NA | NA | NA | NA | NA | NA |
| EIF4EBP2 | 602224 | NA | NA | NA | NA | 593.566 | −5.722133 | uc001jrb.3 |
| EIF5AL1 | NA | 840.144 | −9.244323 | uc009xrx.3 | chr10: 81272871- | NA | NA | NA |
| | | | | | 81276192 | | | |
| ELAVL4 | 168360 | NA | NA | NA | NA | NA | NA | NA |
| ELF2 | NA | 577.819 | −5.858704 | uc011chc.1 | chr4: 139978871- | NA | NA | NA |
| | | | | | 139980136 | | | |
| ELF5 | 605169 | 674.87 | −2.896435 | uc001mvo.1 | chr11: 34500342- | NA | NA | NA |
| | | | | | 34501764 | | | |
| ELK4 | 600246 | NA | NA | NA | NA | NA | NA | NA |
| ELL3 | 609885 | NA | NA | NA | NA | NA | NA | NA |
| ELMOD1 | 615456 | 465.619 | −6.414065 | uc001pjm.3 | chr11: 107535924- | NA | NA | NA |
| | | | | | 107537505 | | | |
| ELMOD2 | 610196 | 1252.62 | −19.980105 | uc003iik.3 | chr4: 141471530- | NA | NA | NA |
| | | | | | 141474924 | | | |
| ELN | 130160 | NA | NA | NA | NA | 313.059 | −5.127891 | uc003tzn.3 |
| ELOVL7 | 614451 | NA | NA | NA | NA | 497.512 | −6.095497 | uc011cqo.2 |
| ELP2 | 616054 | NA | NA | NA | NA | 289.598 | −6.510954 | uc002kzk.2 |
| ELP4 | 606985 | 200.217 | −4.961186 | uc001mtb.3 | chr11: 31805073- | NA | NA | NA |
| | | | | | 31805329 | | | |
| ELSPBP1 | 607443 | NA | NA | NA | NA | 587.068 | −7.482145 | uc002pht.3 |
| EMBP1 | NA | 1149.42 | −9.58363 | uc009wht.1 | chr1: 121310592- | 228.346 | −3.984669 | uc009wht.1 |
| | | | | | 121313686 | | | |
| EMC10 | 614545 | 617.505 | −7.963835 | uc002psk.3 | chr19: 50985406- | NA | NA | NA |
| | | | | | 50986608 | | | |
| EMC2 | 607722 | 708.434 | −9.793651 | uc003ymw.1 | chr8: 109498828- | NA | NA | NA |
| | | | | | 109499136 | | | |
| EMC3 | NA | 793.002 | −8.813713 | uc003buo.3 | chr3: 10010345- | NA | NA | NA |
| | | | | | 10011270 | | | |
| EMC4 | 616245 | NA | NA | NA | NA | 375.083 | −5.248158 | uc001zhq.3 |
| EMCN | 608350 | NA | NA | NA | NA | NA | NA | NA |
| EMILIN2 | 608928 | 422.655 | −4.750617 | uc002kln.3 | chr18: 2913403- | NA | NA | NA |
| | | | | | 2914090 | | | |
| EML1 | 602033 | NA | NA | NA | NA | NA | NA | NA |
| EML4 | 607442 | 272.079 | −6.456969 | uc002rsh.4 | chr2: 42488442- | NA | NA | NA |
| | | | | | 42488639 | | | |
| EMP2 | 602334 | 999.453 | −17.202225 | uc002czx.3 | chr16: 10622279- | NA | NA | NA |
| | | | | | 10626761 | | | |
| ENDOD1 | NA | NA | NA | NA | NA | 1438.69 | −9.334802 | uc001pfh.3 |
| ENDOV | NA | NA | NA | NA | NA | NA | NA | NA |
| ENGASE | 611898 | 657.935 | −6.600342 | uc002jwu.1 | chr17: 77078329- | NA | NA | NA |
| | | | | | 77078959 | | | |
| ENO2 | 131360 | NA | NA | NA | NA | 380.577 | −6.029878 | uc001qru.1 |
| ENOPH1 | NA | NA | NA | NA | NA | 390.545 | −14.687345 | uc003hmv.3 |
| ENPEP | 138297 | 651.452 | −7.654804 | uc003iab.4 | chr4: 111482715- | NA | NA | NA |
| | | | | | 111484493 | | | |
| ENPP2 | 601060 | 1477.21 | −15.956141 | uc011lic.2 | chr8: 120569317- | 868.132 | −10.83832 | uc011lic.2 |
| | | | | | 120569760 | | | |
| ENPP3 | 602182 | 713.996 | −11.633901 | uc003qcu.4 | chr6: 132068097- | NA | NA | NA |
| | | | | | 132068550 | | | |
| ENPP4 | NA | 484.053 | −4.948872 | uc003oxy.3 | chr6: 46111378- | NA | NA | NA |
| | | | | | 46114436 | | | |
| ENSA | 603061 | NA | NA | NA | NA | 483.935 | −6.252863 | uc001evf.3 |
| ENTHD1 | NA | 521.358 | −7.772913 | uc003ayg.3 | chr22: 40139049- | NA | NA | NA |
| | | | | | 40139683 | | | |
| ENTPD1 | 601752 | NA | NA | NA | NA | NA | NA | NA |
| ENTPD4 | 607577 | 280.747 | −4.013073 | uc011kzv.1 | chr8: 23291606- | NA | NA | NA |
| | | | | | 23291825 | | | |
| ENY2 | NA | NA | NA | NA | NA | 748.503 | −12.044533 | uc003ync.3 |
| EOGT | 614789 | NA | NA | NA | NA | NA | NA | NA |
| EP300 | 602700 | 850.608 | −10.309157 | uc003azl.4 | chr22: 41574961- | NA | NA | NA |
| | | | | | 41576081 | | | |
| EP400NL | NA | NA | NA | NA | NA | 834.309 | −5.450347 | uc001ujs.4 |
| EPB41L1 | 602879 | NA | NA | NA | NA | 479.244 | −5.878843 | uc002xeu.3 |
| EPB41L4B | 610340 | NA | NA | NA | NA | 820.291 | −5.679652 | uc004bdz.1 |
| EPB41L5 | 611730 | NA | NA | NA | NA | NA | NA | NA |
| EPDR1 | NA | 228.949 | −6.803556 | uc003tfq.3 | chr7: 37989806- | NA | NA | NA |
| | | | | | 37991542 | | | |
| EPG5 | 615068 | 1550.16 | −10.608766 | uc002lbm.3 | chr18: 43427574- | 760.54 | −6.642035 | uc002lbo.1 |
| | | | | | 43432431 | | | |
| EPHA2 | 176946 | NA | NA | NA | NA | 756.654 | −6.354258 | uc001aya.2 |
| EPHA3 | 179611 | 409.756 | −6.894712 | uc003dqx.1 | chr3: 89448657- | NA | NA | NA |
| | | | | | 89449495 | | | |
| EPHA4 | 602188 | NA | NA | NA | NA | 655.497 | −4.767512 | uc002vmq.3 |
| EPHA5 | 600004 | NA | NA | NA | NA | NA | NA | NA |
| EPHA7 | 602190 | NA | NA | NA | NA | 735.645 | −6.579983 | uc003poe.3 |
| EPHX3 | NA | NA | NA | NA | NA | 432.138 | −8.999419 | uc002nap.3 |
| EPHX4 | NA | 498.656 | −5.830931 | uc001don.2 | chr1: 92528844- | NA | NA | NA |
| | | | | | 92529093 | | | |
| EPM2A | 607566 | NA | NA | NA | NA | NA | NA | NA |
| EPN2 | 607263 | 320.064 | −6.42791 | uc002gvd.4 | chr17: 19237568- | NA | NA | NA |
| | | | | | 19240028 | | | |
| EPPK1 | 607553 | 1751.77 | −20.879433 | uc003zaa.1 | chr8: 144939912- | NA | NA | NA |
| | | | | | 144940158 | | | |
| EPRS | 138295 | NA | NA | NA | NA | NA | NA | NA |
| EPS15 | 600051 | 1288.32 | −13.627571 | uc001csp.3 | chr1: 51819935- | NA | NA | NA |
| | | | | | 51822371 | | | |
| EPS8 | 600206 | 1867.58 | −14.287032 | uc010shv.2 | chr12: 15773075- | NA | NA | NA |
| | | | | | 15774250 | | | |
| EPS8L2 | 614988 | 984.948 | −8.124649 | uc010qwj.1 | chr11: 721940- | NA | NA | NA |
| | | | | | 722302 | | | |
| EPYC | 601657 | 266.888 | −7.274096 | uc001tbk.3 | chr12: 91357456- | NA | NA | NA |
| | | | | | 91357932 | | | |
| ERAP1 | 606832 | NA | NA | NA | NA | 1409.37 | −10.238285 | uc003kml.3 |
| ERBB4 | 600543 | 613.759 | −7.258569 | uc002veg.1 | chr2: 212240442- | NA | NA | NA |
| | | | | | 212248339 | | | |
| ERC1 | 607127 | 714.979 | −6.427947 | uc001qjb.2 | chr12: 1599397- | 829.795 | −9.264101 | uc009zdp.3 |
| | | | | | 1605099 | | | |
| ERCC3 | 133510 | 646.992 | −6.16928 | uc002toe.1 | chr2: 128014866- | 465.09 | −3.739966 | uc010fly.3 |
| | | | | | 128015171 | | | |
| ERCC4 | 133520 | 793.641 | −7.814146 | uc010bva.3 | chr16: 14026160- | 741.777 | −8.71745 | uc010bva.3 |
| | | | | | 14027104 | | | |
| ERCC6L | 300687 | NA | NA | NA | NA | 327.312 | −5.6571 | uc004eap.1 |
| ERCC8 | 609412 | NA | NA | NA | NA | 437.933 | −6.702474 | uc003jsn.3 |
| EREG | 602061 | 825.373 | −15.782308 | uc003hid.3 | chr4: 75246859- | NA | NA | NA |
| | | | | | 75247703 | | | |
| ERGIC3 | NA | 405.955 | −6.193279 | NA | NA | 476.063 | −4.946302 | uc010zvg.2 |
| ERI1 | 608739 | 457.734 | −7.81021 | uc003wsk.2 | chr8: 8887545- | NA | NA | NA |
| | | | | | 8890849 | | | |
| ERI2 | NA | NA | NA | NA | NA | 227.936 | −5.680285 | uc010bwh.2 |
| ERICH3 | NA | NA | NA | NA | NA | 669.518 | −8.271131 | uc001dgi.4 |
| ERMAP | 609017 | 573.695 | −7.841324 | uc010ojw.1 | chr1: 43304655- | 745.997 | −6.737641 | uc010ojw.1 |
| | | | | | 43305384 | | | |
| ERMN | 610072 | NA | NA | NA | NA | 1210.75 | −5.457567 | uc010zcj.2 |
| ERP29 | 602287 | NA | NA | NA | NA | NA | NA | NA |
| ERP44 | 609170 | 994.64 | −13.737964 | uc004bam.3 | chr9: 102741463- | NA | NA | NA |
| | | | | | 102744893 | | | |
| ESCO2 | 609353 | NA | NA | NA | NA | 263.301 | −6.931236 | uc003xgg.3 |
| ESF1 | NA | NA | NA | NA | NA | 466.439 | −5.455059 | uc002woj.3 |
| ESPL1 | 604143 | 385.492 | −4.45297 | uc001scj.2 | chr12: 53687259- | NA | NA | NA |
| | | | | | 53687427 | | | |
| ESRRA | 601998 | NA | NA | NA | NA | NA | NA | NA |
| ESX1 | 300154 | 292.657 | −7.840127 | uc004ely.3 | chrX: 103494719- | NA | NA | NA |
| | | | | | 103494908 | | | |
| ESYT2 | 616691 | 695.412 | −11.607894 | uc003wnz.1 | chr7: 158523689- | NA | NA | NA |
| | | | | | 158526897 | | | |
| ETFB | 130410 | NA | NA | NA | NA | 378.419 | −5.439337 | uc002pwg.3 |
| ETNK1 | 609858 | 530.996 | −3.667603 | uc001rfs.3 | chr12: 22797239- | NA | NA | NA |
| | | | | | 22797349 | | | |
| ETV1 | 600541 | 937.692 | −3.075469 | uc021zzw.1 | chr7: 13946055- | 1074.2 | −5.283375 | uc021zzt.1 |
| | | | | | 13946137 | | | |
| ETV5 | 601600 | 437.962 | −5.857318 | uc003fpy.3 | chr3: 185764106- | NA | NA | NA |
| | | | | | 185766427 | | | |
| EVC2 | 607261 | NA | NA | NA | NA | 535.229 | −7.119264 | uc003gij.3 |
| EVI5L | NA | 521.651 | −5.143917 | uc002min.3 | chr19: 7928589- | NA | NA | NA |
| | | | | | 7929862 | | | |
| EVPL | 601590 | 572.197 | −7.400201 | uc002jqi.2 | chr17: 74002927- | 385.5 | −5.285101 | uc002jqi.2 |
| | | | | | 74003183 | | | |
| EXOC4 | 608185 | 1087.54 | −8.103812 | uc003vri.3 | chr7: 133164901- | NA | NA | NA |
| | | | | | 133165805 | | | |
| EXOC6 | 609672 | 344.392 | −7.398995 | uc010qnr.2 | chr10: 94818113- | NA | NA | NA |
| | | | | | 94819251 | | | |
| EXOC7 | 608163 | NA | NA | NA | NA | 753.122 | −7.058542 | uc002jqp.2 |
| EXOC8 | 615283 | 1017.64 | −10.284304 | uc001huq.3 | chr1: 231468482- | NA | NA | NA |
| | | | | | 231471313 | | | |
| EXOSC10 | 605960 | NA | NA | NA | NA | 767.099 | −8.251079 | uc009vmy.1 |
| EXOSC8 | 606019 | NA | NA | NA | NA | 272.46 | −11.434782 | uc001uwa.3 |
| EXOSC9 | 606180 | NA | NA | NA | NA | 523.38 | −10.641849 | uc003idz.3 |
| EYA4 | 603550 | 416.615 | −5.210592 | uc011ecq.2 | chr6: 133849944- | NA | NA | NA |
| | | | | | 133853258 | | | |
| EZH1 | 601674 | 339.421 | −5.250511 | uc010cyo.1 | chr17: 40855441- | 616.738 | −7.080547 | uc002iaz.3 |
| | | | | | 40855743 | | | |
| EZH2 | 601573 | NA | NA | NA | NA | 333.636 | −6.693194 | uc022aov.1 |
| EZR | 123900 | NA | NA | NA | NA | 485.701 | −6.2637 | uc011efr.2 |
| F11R | 605721 | NA | NA | NA | NA | 551.86 | −5.244337 | uc010pjv.2 |
| FAAH2 | 300654 | NA | NA | NA | NA | NA | NA | NA |
| FABP1 | 134650 | NA | NA | NA | NA | NA | NA | NA |
| FABP4 | 600434 | NA | NA | NA | NA | NA | NA | NA |
| FABP7 | 602965 | 236.097 | −7.737058 | uc003pzd.3 | chr6: 123102493- | 1014.81 | −4.286046 | uc003pzd.3 |
| | | | | | 123104039 | | | |
| FADD | 602457 | 904.1 | −7.115195 | uc001opm.2 | chr11: 70052580- | NA | NA | NA |
| | | | | | 70053508 | | | |
| FAF2 | NA | NA | NA | NA | NA | 933.351 | −16.476217 | uc003mej.4 |
| FAHD1 | 616320 | NA | NA | NA | NA | 248.59 | −2.30319 | uc002cnc.1 |
| FAM104A | NA | 553.647 | −4.06257 | uc002jji.4 | chr17: 71203492- | NA | NA | NA |
| | | | | | 71205667 | | | |
| FAM110B | 611394 | 251.28 | −7.606873 | uc003xtj.1 | chr8: 59059903- | 215.506 | −5.081129 | uc003xtj.1 |
| | | | | | 59062277 | | | |
| FAM111A | 615292 | NA | NA | NA | NA | NA | NA | NA |
| FAM117B | NA | 643.13 | −3.809107 | uc010zhw.2 | chr2: 203589757- | 2794.24 | −6.317217 | uc010zhw.2 |
| | | | | | 203589955 | | | |
| FAM120AOS | NA | 953.087 | −5.703267 | uc004atu.4 | chr9: 96208782- | NA | NA | NA |
| | | | | | 96209892 | | | |
| FAM122B | NA | NA | NA | NA | NA | 201.039 | −5.346047 | uc004ext.3 |
| FAM126A | 610531 | NA | NA | NA | NA | NA | NA | NA |
| FAM126B | NA | NA | NA | NA | NA | NA | NA | NA |
| FAM134C | 616498 | 219.038 | −4.195707 | uc002ial.2 | chr17: 40733057- | NA | NA | NA |
| | | | | | 40733830 | | | |
| FAM135A | NA | 852.277 | −7.021129 | uc003pfh.3 | chr6: 71269625- | 618.164 | −6.716301 | uc003pfh.3 |
| | | | | | 71270877 | | | |
| FAM135B | NA | NA | NA | NA | NA | 355.14 | −7.283888 | uc003yva.3 |
| FAM13B | 609371 | NA | NA | NA | NA | 2570.92 | −19.566165 | uc003lbz.2 |
| FAM13C | NA | 288.584 | −5.968631 | uc001jkn.3 | chr10: 61005889- | NA | NA | NA |
| | | | | | 61007373 | | | |
| FAM149A | NA | 356.623 | −5.742345 | uc011cla.1 | chr4: 187088443- | 362.14 | −8.913186 | uc010isj.2 |
| | | | | | 187088484 | | | |
| FAM149B1 | NA | NA | NA | NA | NA | 484.555 | −6.916269 | uc009xqz.3 |
| FAM151B | NA | NA | NA | NA | NA | 1467.52 | −12.066363 | uc003kgv.2 |
| FAM153C | NA | 981.353 | −11.253161 | uc011dge.2 | chr5: 177475666- | NA | NA | NA |
| | | | | | 177476088 | | | |
| FAM160A1 | NA | 602.036 | −9.943427 | uc003imj.2 | chr4: 152583907- | NA | NA | NA |
| | | | | | 152584784 | | | |
| FAM160A2 | NA | 275.455 | −4.624329 | uc001mck.4 | chr11: 6232564- | NA | NA | NA |
| | | | | | 6232735 | | | |
| FAM162A | 608017 | NA | NA | NA | NA | 369.079 | −5.656457 | uc011bjq.1 |
| FAM163A | 611727 | NA | NA | NA | NA | NA | NA | NA |
| FAM166A | NA | 334.37 | −3.71663 | uc004cmi.1 | chr9: 140138037- | 406.028 | −6.281993 | uc004cmi.1 |
| | | | | | 140138151 | | | |
| FAM166B | NA | NA | NA | NA | NA | 239.206 | −4.37522 | uc010mkr.3 |
| FAM168B | NA | NA | NA | NA | NA | NA | NA | NA |
| FAM169A | 615769 | NA | NA | NA | NA | 732.258 | −11.970916 | uc010izm.3 |
| FAM171B | NA | NA | NA | NA | NA | 811.201 | −10.509981 | uc002upr.1 |
| FAM172A | NA | 283.385 | −4.499109 | uc010jbd.3 | chr5: 92953431- | 427.233 | −8.210142 | uc010jbd.3 |
| | | | | | 92956692 | | | |
| FAM172BP | NA | NA | NA | NA | NA | 208.256 | −4.779559 | uc003duy.1 |
| FAM175A | 611143 | NA | NA | NA | NA | 473.443 | −2.969149 | uc003hou.2 |
| FAM177A1 | NA | 1488.87 | −10.861595 | uc001wsp.3 | chr14: 35550435- | NA | NA | NA |
| | | | | | 35552589 | | | |
| FAM177B | NA | NA | NA | NA | NA | 398.801 | −5.102033 | uc001hnt.3 |
| FAM179A | NA | 312.473 | −4.990424 | uc002rms.1 | chr2: 29268405- | 965.487 | −9.350703 | uc002rms.1 |
| | | | | | 29270413 | | | |
| FAM179B | NA | 433.834 | −5.463865 | uc010anb.1 | chr14: 45465144- | 961.905 | −2.876145 | uc001wvu.3 |
| | | | | | 45466607 | | | |
| FAM180A | NA | 766.331 | −9.081762 | uc003vtd.3 | chr7: 135418394- | NA | NA | NA |
| | | | | | 135418722 | | | |
| FAM184A | NA | NA | NA | NA | NA | 481.302 | −5.598799 | uc003pyj.3 |
| FAM189A2 | 607710 | NA | NA | NA | NA | 307.375 | −5.371038 | uc010moo.1 |
| FAM189B | NA | NA | NA | NA | NA | 496.217 | −6.502594 | uc001fjq.1 |
| FAM193A | NA | NA | NA | NA | NA | NA | NA | NA |
| FAM198A | NA | 1267.21 | −5.103611 | uc003cmp.4 | chr3: 43097879- | NA | NA | NA |
| | | | | | 43099207 | | | |
| FAM198B | NA | 1414.15 | −7.870705 | uc003ipp.4 | chr4: 159045732- | NA | NA | NA |
| | | | | | 159048558 | | | |
| FAM199X | NA | NA | NA | NA | NA | NA | NA | NA |
| FAM200A | NA | 312.101 | −4.982461 | uc003ura.3 | chr7: 99143923- | NA | NA | NA |
| | | | | | 99144308 | | | |
| FAM208A | 616493 | NA | NA | NA | NA | 1255.92 | −3.565784 | uc003dib.4 |
| FAM20B | 611063 | NA | NA | NA | NA | 370.258 | −4.84583 | uc001gmc.3 |
| FAM210B | NA | NA | NA | NA | NA | NA | NA | NA |
| FAM213A | NA | 278.129 | −5.954095 | uc021pux.1 | chr10: 82191856- | 1167.11 | −13.056718 | uc021pux.1 |
| | | | | | 82192753 | | | |
| FAM213B | NA | 870.187 | −4.949832 | uc021oex.1 | chr1: 2520919- | NA | NA | NA |
| | | | | | 2522908 | | | |
| FAM215A | NA | 536.705 | −5.76815 | uc010wiq.1 | chr17: 41995024- | NA | NA | NA |
| | | | | | 41995355 | | | |
| FAM216A | NA | NA | NA | NA | NA | 679.05 | −5.4696 | uc001tqu.4 |
| FAM216B | NA | 1080.2 | −7.953717 | uc010tfk.2 | chr13: 43362927- | NA | NA | NA |
| | | | | | 43365685 | | | |
| FAM219B | NA | NA | NA | NA | NA | NA | NA | NA |
| FAM21C | 613631 | 1273.94 | −11.847784 | uc001jcu.3 | chr10: 46287849- | NA | NA | NA |
| | | | | | 46288412 | | | |
| FAM221A | NA | 1477.99 | −17.796224 | uc003swo.4 | chr7: 23741784- | NA | NA | NA |
| | | | | | 23742269 | | | |
| FAM222B | NA | 619.993 | −6.315861 | uc002hct.1 | chr17: 27082996- | NA | NA | NA |
| | | | | | 27085287 | | | |
| FAM227B | NA | NA | NA | NA | NA | NA | NA | NA |
| FAM24A | NA | 205.074 | −5.777951 | uc001lgv.3 | chr10: 124672471- | 1433.65 | −10.131519 | uc001lgv.3 |
| | | | | | 124672627 | | | |
| FAM24B | NA | 497.151 | −8.072033 | uc001lgt.3 | chr10: 124608610- | NA | NA | NA |
| | | | | | 124608762 | | | |
| FAM26E | NA | NA | NA | NA | NA | NA | NA | NA |
| FAM3D | 608619 | NA | NA | NA | NA | NA | NA | NA |
| FAM46A | 611357 | 612.19 | −5.988155 | uc003pjf.3 | chr6: 82455447- | NA | NA | NA |
| | | | | | 82459411 | | | |
| FAM47A | NA | 1618.07 | −17.231484 | uc004ddg.3 | chrX: 34147869- | 1183.69 | −6.095428 | uc004ddg.3 |
| | | | | | 34148019 | | | |
| FAM47B | NA | 1470.7 | −14.785593 | uc004ddi.2 | chrX: 34962887- | 1189.87 | −8.055785 | uc004ddi.2 |
| | | | | | 34963034 | | | |
| FAM47C | NA | 1432.13 | −15.900546 | uc004ddl.2 | chrX: 37029592- | NA | NA | NA |
| | | | | | 37029739 | | | |
| FAM49A | NA | NA | NA | NA | NA | NA | NA | NA |
| FAM50B | 614686 | NA | NA | NA | NA | NA | NA | NA |
| FAM57A | 611627 | NA | NA | NA | NA | NA | NA | NA |
| FAM65C | NA | 567.272 | −5.755202 | uc002xvm.3 | chr20: 49202647- | NA | NA | NA |
| | | | | | 49203768 | | | |
| FAM69A | 614542 | NA | NA | NA | NA | NA | NA | NA |
| FAM72B | 614711 | NA | NA | NA | NA | 538.565 | −5.575271 | uc009whp.3 |
| FAM73A | NA | 278.309 | −7.997543 | uc001dhy.1 | chr1: 78324782- | 1584.09 | −8.287003 | uc001dhy.1 |
| | | | | | 78327860 | | | |
| FAM73B | NA | 681.683 | −0.760026 | uc011mbn.1 | chr9: 131830813- | 1476.26 | −5.680441 | uc011mbn.1 |
| | | | | | 131831254 | | | |
| FAM76A | NA | NA | NA | NA | NA | NA | NA | NA |
| FAM76B | NA | NA | NA | NA | NA | 853.112 | −6.96541 | uc001pfn.2 |
| FAM81A | NA | NA | NA | NA | NA | NA | NA | NA |
| FAM81B | NA | 748.733 | −8.921045 | uc003kla.1 | chr5: 94785987- | NA | NA | NA |
| | | | | | 94786144 | | | |
| FAM83D | NA | 713.008 | −2.777772 | uc002xjf.3 | chr20: 37570803- | 874.944 | −9.617066 | uc002xjg.3 |
| | | | | | 37574842 | | | |
| FAM89A | NA | NA | NA | NA | NA | 671.467 | −6.979481 | uc001hui.2 |
| FAM91A1 | NA | 328.638 | −9.606683 | uc011lik.1 | chr8: 124822631- | 1087.17 | −7.07014 | uc003yqv.3 |
| | | | | | 124822719 | | | |
| FAM92A1 | NA | NA | NA | NA | NA | 590.992 | −8.787469 | uc022ayc.1 |
| FAM9A | 300477 | NA | NA | NA | NA | 722.141 | −8.760234 | uc022bsk.1 |
| FAM9C | 300479 | 1019.8 | −3.303881 | uc004cvg.3 | chrX: 13053736- | NA | NA | NA |
| | | | | | 13056559 | | | |
| FANCA | 607139 | NA | NA | NA | NA | NA | NA | NA |
| FANCD2 | 613984 | 665.139 | −4.543067 | uc003buv.3 | chr3: 10081561- | 618.67 | −7.048315 | uc003buv.3 |
| | | | | | 10083246 | | | |
| FANCE | 613976 | 529.387 | −5.110535 | uc003oko.1 | chr6: 35434123- | NA | NA | NA |
| | | | | | 35434881 | | | |
| FANCF | 613897 | 552.323 | −7.425937 | uc001mql.1 | chr11: 22644079- | NA | NA | NA |
| | | | | | 22646231 | | | |
| FANCM | 609644 | NA | NA | NA | NA | NA | NA | NA |
| FANK1 | 611640 | NA | NA | NA | NA | 511.917 | −6.870795 | uc001ljh.4 |
| FAP | 600403 | NA | NA | NA | NA | 842.047 | −5.856117 | uc010fpc.3 |
| FARP1 | 602654 | 275.098 | −4.390561 | uc001vnh.3 | chr13: 99100572- | 557.756 | −6.863112 | uc001vnh.3 |
| | | | | | 99102023 | | | |
| FASTKD1 | NA | 950.169 | −13.085491 | uc002uev.4 | chr2: 170386263- | NA | NA | NA |
| | | | | | 170386334 | | | |
| FASTKD2 | 612322 | NA | NA | NA | NA | 410.57 | −6.990853 | uc002vbu.3 |
| FASTKD3 | NA | NA | NA | NA | NA | NA | NA | NA |
| FAT2 | 604269 | 574.531 | −3.225304 | uc003lud.4 | chr5: 150883653- | 869.533 | −6.111992 | uc003lud.4 |
| | | | | | 150885125 | | | |
| FAU | 134690 | NA | NA | NA | NA | 588.661 | −6.011357 | uc001ocx.3 |
| FAXDC2 | NA | NA | NA | NA | NA | NA | NA | NA |
| FBLL1 | NA | 1006.28 | −12.356562 | uc011dep.2 | chr5: 167957515- | 1737.79 | −11.180242 | uc011dep.2 |
| | | | | | 167957639 | | | |
| FBLN2 | 135821 | 899.288 | −12.747037 | uc011auz.2 | chr3: 13679420- | 441.463 | −4.817318 | uc011auz.2 |
| | | | | | 13679922 | | | |
| FBN2 | 612570 | NA | NA | NA | NA | NA | NA | NA |
| FBP1 | 611570 | NA | NA | NA | NA | NA | NA | NA |
| FBP2 | 603027 | NA | NA | NA | NA | 478.448 | −7.841707 | uc004auv.3 |
| FBXL13 | 609080 | NA | NA | NA | NA | NA | NA | NA |
| FBXL17 | 609083 | 843.43 | −9.91767 | uc003kon.4 | chr5: 107213952- | NA | NA | NA |
| | | | | | 107216563 | | | |
| FBXL22 | 609088 | NA | NA | NA | NA | NA | NA | NA |
| FBXL3 | 605653 | NA | NA | NA | NA | 1019.68 | −14.675279 | uc001vkd.3 |
| FBXL4 | 605654 | 321.422 | −8.102104 | uc003ppf.1 | chr6: 99321601- | 452.089 | −7.86284 | uc010kcp.3 |
| | | | | | 99322153 | | | |
| FBXL5 | 605655 | 807.02 | −10.998024 | uc010idw.2 | chr4: 15606007- | NA | NA | NA |
| | | | | | 15607345 | | | |
| FBXL6 | 609076 | 392.601 | −4.01076 | uc003zbz.3 | chr8: 145579088- | NA | NA | NA |
| | | | | | 145579190 | | | |
| FBXO11 | 607871 | 776.295 | −4.855062 | uc010fbk.3 | chr2: 48034059- | NA | NA | NA |
| | | | | | 48035256 | | | |
| FBXO15 | 609093 | NA | NA | NA | NA | NA | NA | NA |
| FBXO21 | 609095 | 376.19 | −7.212301 | uc001twj.3 | chr12: 117581585- | NA | NA | NA |
| | | | | | 117583891 | | | |
| FBXO22 | 609096 | 346.582 | −5.387853 | uc002bbj.2 | chr15: 76222428- | NA | NA | NA |
| | | | | | 76223208 | | | |
| FBXO25 | 609098 | 389.569 | −7.067585 | uc003wox.3 | chr8: 418805- | NA | NA | NA |
| | | | | | 419875 | | | |
| FBXO30 | 609101 | NA | NA | NA | NA | NA | NA | NA |
| FBXO33 | 609103 | 743.068 | −11.858334 | uc001wvk.3 | chr14: 39865577- | NA | NA | NA |
| | | | | | 39868719 | | | |
| FBXO39 | 609106 | NA | NA | NA | NA | NA | NA | NA |
| FBXO42 | 609109 | 674.74 | −7.801615 | uc001ayf.3 | chr1: 16576559- | NA | NA | NA |
| | | | | | 16577164 | | | |
| FBXO43 | 609110 | NA | NA | NA | NA | 455.812 | −6.351615 | uc003yjd.3 |
| FBXO5 | 606013 | NA | NA | NA | NA | 519.849 | −7.21788 | uc003qpg.3 |
| FBXO7 | 605648 | 921.064 | −9.899164 | uc003amq.3 | chr22: 32894518- | NA | NA | NA |
| | | | | | 32894818 | | | |
| FBXW11 | 605651 | 704.871 | −6.476772 | uc011dey.1 | chr5: 171288556- | NA | NA | NA |
| | | | | | 171291105 | | | |
| FBXW7 | 606278 | 825.861 | −11.042294 | uc003imu.3 | chr4: 153332312- | 787.213 | −9.381955 | uc003imu.3 |
| | | | | | 153332424 | | | |
| FCER1A | 147140 | NA | NA | NA | NA | 956.17 | −6.981329 | uc001ftq.3 |
| FCER1G | 147139 | NA | NA | NA | NA | 1020.61 | −8.878375 | uc001fyz.1 |
| FCGR1A | 146760 | 817.451 | −6.342293 | uc001esp.4 | chr1: 149763074- | NA | NA | NA |
| | | | | | 149764074 | | | |
| FCGR2A | 146790 | 742.747 | −9.566993 | uc001gam.3 | chr1: 161487939- | NA | NA | NA |
| | | | | | 161489360 | | | |
| FCHO2 | 613438 | NA | NA | NA | NA | 540.378 | −4.661996 | uc011csk.1 |
| FCHSD2 | NA | NA | NA | NA | NA | NA | NA | NA |
| FCRL3 | 606510 | 631.85 | −5.997229 | uc001fra.3 | chr1: 157647978- | NA | NA | NA |
| | | | | | 157648499 | | | |
| FCRLB | 609251 | 982.874 | −3.068911 | uc001gbh.3 | chr1: 161697453- | NA | NA | NA |
| | | | | | 161697933 | | | |
| FDFT1 | 184420 | NA | NA | NA | NA | NA | NA | NA |
| FDX1 | 103260 | NA | NA | NA | NA | 1504.72 | −8.931724 | uc001pkx.3 |
| FDXACB1 | NA | NA | NA | NA | NA | 661.499 | −6.056115 | uc001pmc.4 |
| FEN1 | 600393 | NA | NA | NA | NA | 481.204 | −5.556089 | uc001nsg.3 |
| FER1L5 | NA | 407.464 | −5.978523 | uc002swr.2 | chr2: 97317767- | NA | NA | NA |
| | | | | | 97320635 | | | |
| FERMT1 | 607900 | NA | NA | NA | NA | 430.287 | −6.110874 | uc002wmq.3 |
| FEZ1 | 604825 | 1998.29 | −10.408843 | uc001qbx.3 | chr11: 125315641- | NA | NA | NA |
| | | | | | 125315990 | | | |
| FGB | 134830 | 358.31 | −3.541012 | uc003ioa.4 | chr4: 155491803- | 998.238 | −13.942872 | uc003ioa.4 |
| | | | | | 155493915 | | | |
| FGD4 | 611104 | NA | NA | NA | NA | 253.853 | −3.480662 | uc001rlc.3 |
| FGF1 | 131220 | 653.205 | −5.469545 | uc003lmq.3 | chr5: 141971743- | NA | NA | NA |
| | | | | | 141974854 | | | |
| FGF14 | 601515 | NA | NA | NA | NA | 640.936 | −5.998116 | uc001vpe.2 |
| FGF20 | 605558 | 861.158 | −7.06114 | uc003wxc.1 | chr8: 16850334- | NA | NA | NA |
| | | | | | 16850580 | | | |
| FGF21 | 609436 | 1643.77 | −6.711043 | uc002pkn.1 | chr19: 49261478- | NA | NA | NA |
| | | | | | 49261582 | | | |
| FGF7 | 148180 | NA | NA | NA | NA | 767.527 | −9.129266 | uc001zxn.3 |
| FGFR1OP | 605392 | NA | NA | NA | NA | NA | NA | NA |
| FGL1 | 605776 | NA | NA | NA | NA | 939.966 | −5.431716 | uc003wxx.3 |
| FGL2 | 605351 | NA | NA | NA | NA | 740.261 | −7.243553 | uc003ugb.3 |
| FHL2 | 602633 | 492.159 | −4.682318 | uc002tct.3 | chr2: 105977283- | 1149.03 | −10.829891 | uc002tct.3 |
| | | | | | 105977739 | | | |
| FHL5 | 605126 | NA | NA | NA | NA | NA | NA | NA |
| FIBIN | NA | NA | NA | NA | NA | NA | NA | NA |
| FIGF | 300091 | 378.795 | −4.551301 | uc004cwt.2 | chrX: 15363713- | NA | NA | NA |
| | | | | | 15364254 | | | |
| FIGNL1 | 615383 | 657.614 | −6.886462 | uc003tpb.3 | chr7: 50511832- | 300.538 | −9.155832 | uc003tpb.3 |
| | | | | | 50512960 | | | |
| FILIP1 | 607307 | 291.652 | −5.457279 | uc003phy.1 | chr6: 76004607- | NA | NA | NA |
| | | | | | 76005625 | | | |
| FILIP1L | 612993 | NA | NA | NA | NA | 250.621 | −4.557543 | uc003dtn.3 |
| FIP1L1 | 607686 | 625.106 | −5.446964 | uc011bzt.1 | chr4: 54306937- | 826.105 | −8.115755 | uc003gzy.3 |
| | | | | | 54307150 | | | |
| FIS1 | 609003 | 783.085 | −6.060458 | uc003uyj.4 | chr7: 100882893- | NA | NA | NA |
| | | | | | 100883086 | | | |
| FITM2 | 612029 | 895.259 | −7.561535 | uc002xlr.1 | chr20: 42935197- | NA | NA | NA |
| | | | | | 42935264 | | | |
| FKBP15 | NA | 747.008 | −10.68648 | uc004bgr.2 | chr9: 115927800- | NA | NA | NA |
| | | | | | 115928357 | | | |
| FKBP6 | 604839 | NA | NA | NA | NA | NA | NA | NA |
| FKRP | 606596 | 479.68 | −4.773948 | uc002pfn.2 | chr19: 47260196- | NA | NA | NA |
| | | | | | 47261832 | | | |
| FKSG29 | NA | NA | NA | NA | NA | 1537.06 | −8.738476 | uc010afy.1 |
| FLCN | 607273 | NA | NA | NA | NA | NA | NA | NA |
| FLJ10038 | NA | NA | NA | NA | NA | NA | NA | NA |
| FLJ13224 | NA | NA | NA | NA | NA | 490.542 | −6.237078 | uc001rkf.1 |
| FLJ30679 | NA | 672.059 | −8.836901 | uc010vos.1 | chr16: 86589345- | NA | NA | NA |
| | | | | | 86590905 | | | |
| FLJ33360 | NA | 1207.76 | −8.174818 | uc003jdn.1 | chr5: 6310554- | NA | NA | NA |
| | | | | | 6312482 | | | |
| FLJ44635 | NA | 302.009 | −2.880372 | uc004eal.2 | chrX: 71380103- | 425.448 | −5.199318 | uc004eal.2 |
| | | | | | 71381600 | | | |
| FLNB | 603381 | 1053.27 | −9.12147 | uc003djj.2 | chr3: 58156490- | 982.981 | −9.502373 | uc003djj.2 |
| | | | | | 58157982 | | | |
| FLT4 | 136352 | NA | NA | NA | NA | NA | NA | NA |
| FLVCR1 | 609144 | 699.147 | −9.842755 | uc001hjt.3 | chr1: 213068633- | NA | NA | NA |
| | | | | | 213072705 | | | |
| FMN2 | 606373 | NA | NA | NA | NA | NA | NA | NA |
| FMOD | 600245 | NA | NA | NA | NA | NA | NA | NA |
| FN1 | 135600 | NA | NA | NA | NA | NA | NA | NA |
| FN3KRP | 611683 | 1223.56 | −6.739879 | uc002kfu.3 | chr17: 80685048- | NA | NA | NA |
| | | | | | 80685893 | | | |
| FNBP4 | 615265 | NA | NA | NA | NA | NA | NA | NA |
| FNDC3A | 615794 | NA | NA | NA | NA | 1632.47 | −10.783082 | uc001vcl.1 |
| FNDC3B | 611909 | 1039.45 | −15.759713 | uc003fia.3 | chr3: 172055230- | NA | NA | NA |
| | | | | | 172056104 | | | |
| FNTA | 134635 | 590.746 | −5.443081 | uc003xps.3 | chr8: 42940426- | NA | NA | NA |
| | | | | | 42940932 | | | |
| FOLR1 | 136430 | NA | NA | NA | NA | 514.93 | −5.35717 | uc001orz.2 |
| FOLR2 | 136425 | NA | NA | NA | NA | 428.406 | −4.624517 | uc001ose.4 |
| FOPNL | NA | 752.289 | −12.342722 | uc002dec.1 | chr16: 15959577- | 611.986 | −5.584917 | uc002dec.1 |
| | | | | | 15961296 | | | |
| FOXA3 | 602295 | 2000.62 | −12.034165 | uc002pdr.3 | chr19: 46376317- | NA | NA | NA |
| | | | | | 46377055 | | | |
| FOXK2 | 147685 | NA | NA | NA | NA | 356.128 | −0.602221 | uc002kfm.1 |
| FOXN3 | 602628 | NA | NA | NA | NA | 1868.03 | −18.201903 | uc001xxn.4 |
| FOXP1 | 605515 | NA | NA | NA | NA | 1026.69 | −8.206464 | uc003doi.3 |
| FOXP2 | 605317 | 331.38 | −8.663074 | uc003vgw.3 | chr7: 114268769- | 597.673 | −5.44009 | uc003vgx.2 |
| | | | | | 114269032 | | | |
| FPGS | 136510 | NA | NA | NA | NA | NA | NA | NA |
| FPGT | 603609 | NA | NA | NA | NA | 698.925 | −4.684035 | uc001dgb.2 |
| FRA10AC1 | 608866 | 1703.2 | −1.827337 | uc001kjb.1 | chr10: 95448837- | 569.95 | −8.40106 | uc001kjb.1 |
| | | | | | 95451720 | | | |
| FREM1 | 608944 | NA | NA | NA | NA | NA | NA | NA |
| FREM2 | 608945 | NA | NA | NA | NA | 495.446 | −7.223484 | uc001uww.3 |
| FRG1 | 601278 | 265.75 | −4.307423 | uc003izs.3 | chr4: 190884285- | 1155.47 | −10.400992 | NA |
| | | | | | 190884359 | | | |
| FRG1B | NA | NA | NA | NA | NA | 271.503 | −4.19588 | uc010ztk.1 |
| FRG2EP | NA | NA | NA | NA | NA | NA | NA | NA |
| FRMD4B | NA | 210.192 | −3.533304 | uc003dnu.2 | chr3: 69219146- | 645.832 | −9.020197 | uc003dnu.2 |
| | | | | | 69221011 | | | |
| FRMD6 | 614555 | 1679.68 | −10.35757 | uc001wzc.3 | chr14: 52194748- | NA | NA | NA |
| | | | | | 52197444 | | | |
| FRMD7 | 300628 | NA | NA | NA | NA | 368.446 | −6.15846 | uc022cdy.1 |
| FRMD8 | NA | NA | NA | NA | NA | NA | NA | NA |
| FRMPD2 | 613323 | NA | NA | NA | NA | NA | NA | NA |
| FRMPD2B | NA | NA | NA | NA | NA | NA | NA | NA |
| FRRS1L | 604574 | NA | NA | NA | NA | NA | NA | NA |
| FRS2 | 607743 | 1282.05 | −5.629612 | uc001suy.3 | chr12: 69968736- | 281.453 | −8.049302 | uc001suy.3 |
| | | | | | 69973562 | | | |
| FRYL | NA | 2347.31 | −6.484739 | uc003gyl.1 | chr4: 48619621- | NA | NA | NA |
| | | | | | 48621200 | | | |
| FRZB | 605083 | NA | NA | NA | NA | 703.864 | −8.698223 | uc002upa.2 |
| FSIP1 | 615795 | 242.825 | −5.600257 | uc001zki.3 | chr15: 39892232- | 567.686 | −11.220535 | uc001zki.3 |
| | | | | | 39893080 | | | |
| FSTL3 | 605343 | 453.133 | −4.37522 | uc002lpk.1 | chr19: 681709- | NA | NA | NA |
| | | | | | 683392 | | | |
| FSTL4 | NA | 1384.62 | −7.528398 | uc003kym.1 | chr5: 132532152- | 717.631 | −4.355081 | uc003kym.1 |
| | | | | | 132534786 | | | |
| FTHL17 | 300308 | 822.368 | −6.735242 | uc004dcl.1 | chrX: 31089358- | NA | NA | NA |
| | | | | | 31089518 | | | |
| FTMT | 608847 | NA | NA | NA | NA | NA | NA | NA |
| FTSJ2 | 606906 | NA | NA | NA | NA | NA | NA | NA |
| FUBP1 | 603444 | NA | NA | NA | NA | NA | NA | NA |
| FUBP3 | 603536 | 1360.58 | −10.539367 | uc004bzr.1 | chr9: 133512410- | NA | NA | NA |
| | | | | | 133513739 | | | |
| FUCA1 | 612280 | NA | NA | NA | NA | 355.597 | −6.230619 | uc001bie.3 |
| FUCA2 | 136820 | 593.396 | −7.287561 | uc003qjm.3 | chr6: 143815949- | NA | NA | NA |
| | | | | | 143816843 | | | |
| FUNDC2 | NA | NA | NA | NA | NA | NA | NA | NA |
| FUNDC2P2 | NA | NA | NA | NA | NA | 641.955 | −5.51941 | uc010ffz.1 |
| FUOM | NA | 585.318 | −4.393945 | uc001lms.2 | chr10: 135168658- | NA | NA | NA |
| | | | | | 135169002 | | | |
| FUS | 137070 | 522.526 | −7.680538 | uc010vfi.2 | chr16: 31194165- | NA | NA | NA |
| | | | | | 31194671 | | | |
| FUT10 | NA | NA | NA | NA | NA | NA | NA | NA |
| FUT11 | NA | NA | NA | NA | NA | 478.678 | −5.591052 | uc001juy.1 |
| FUT4 | 104230 | NA | NA | NA | NA | 790.225 | −8.222126 | uc001pez.3 |
| FUT9 | 606865 | 289.782 | −6.659611 | uc003pop.4 | chr6: 96652112- | NA | NA | NA |
| | | | | | 96663488 | | | |
| FUZ | 610622 | 363.437 | −5.612617 | NA | NA | 352.869 | −6.295051 | uc002ppq.2 |
| FXN | 606829 | NA | NA | NA | NA | NA | NA | NA |
| FXYD5 | 606669 | NA | NA | NA | NA | NA | NA | NA |
| FYB | 602731 | 587.639 | −6.635598 | uc003jls.3 | chr5: 39105354- | NA | NA | NA |
| | | | | | 39107544 | | | |
| FZD10 | 606147 | 1070.13 | −6.897438 | uc001uii.3 | chr12: 130649234- | NA | NA | NA |
| | | | | | 130650285 | | | |
| FZD2 | 600667 | 508.326 | −5.105848 | uc002igx.2 | chr17: 42636755- | 449.525 | −4.941699 | uc002igx.2 |
| | | | | | 42638630 | | | |
| FZD4 | 604579 | NA | NA | NA | NA | NA | NA | NA |
| FZD6 | 603409 | 1448.3 | −12.985985 | uc003ylh.3 | chr8: 104343738- | NA | NA | NA |
| | | | | | 104345094 | | | |
| G2E3 | 611299 | 590.655 | −4.641665 | uc010tpe.1 | chr14: 31077119- | NA | NA | NA |
| | | | | | 31077275 | | | |
| G3BP2 | NA | 328.283 | −6.619905 | uc003hir.3 | chr4: 76567953- | 1101.64 | −13.40213 | uc003hir.3 |
| | | | | | 76570613 | | | |
| GAB3 | 300482 | NA | NA | NA | NA | 897.779 | −6.792263 | uc004fmj.1 |
| GAB4 | NA | NA | NA | NA | NA | 557.326 | −6.962161 | uc002zlw.3 |
| GABPB1 | 600610 | 846.739 | −12.765153 | uc001zyc.3 | chr15: 50578077- | 1198.17 | −10.664636 | uc001zyc.3 |
| | | | | | 50578216 | | | |
| GABRA1 | 137160 | NA | NA | NA | NA | 964.987 | −12.78744 | uc010jiw.3 |
| GABRA6 | 137143 | NA | NA | NA | NA | 1094.08 | −7.291649 | uc003lyu.2 |
| GABRG2 | 137164 | NA | NA | NA | NA | 1011.24 | −11.912703 | uc003lyy.4 |
| GABRR1 | 137161 | 372.088 | −7.282323 | uc003pna.2 | chr6: 89887223- | NA | NA | NA |
| | | | | | 89888488 | | | |
| GADD45A | 126335 | 348.53 | −7.805044 | uc009wbc.2 | chr1: 68153381- | NA | NA | NA |
| | | | | | 68154021 | | | |
| GALC | 606890 | 990.353 | −7.03664 | uc001xvt.3 | chr14: 88399358- | 592.377 | −9.367979 | uc001xvt.3 |
| | | | | | 88401075 | | | |
| GALNS | 612222 | NA | NA | NA | NA | 642.369 | −7.30881 | uc002fma.3 |
| GALNT13 | 608369 | 647.707 | −2.266375 | uc002tyr.4 | chr2: 155307064- | 959.633 | −6.087396 | uc002tyr.4 |
| | | | | | 155310489 | | | |
| GALNT15 | 615131 | 1552.45 | −9.585602 | uc003caq.4 | chr3: 16269008- | NA | NA | NA |
| | | | | | 16271253 | | | |
| GALNT3 | 601756 | 499.705 | −8.586398 | uc010fph.1 | chr2: 166604313- | NA | NA | NA |
| | | | | | 166605290 | | | |
| GALNT8 | 176257 | NA | NA | NA | NA | 607.972 | −4.765543 | NA |
| GALP | 611178 | NA | NA | NA | NA | 771.237 | −6.893114 | uc002qmo.1 |
| GALR1 | 600377 | NA | NA | NA | NA | NA | NA | NA |
| GAPT | NA | NA | NA | NA | NA | NA | NA | NA |
| GAR1 | 606468 | NA | NA | NA | NA | NA | NA | NA |
| GARNL3 | NA | NA | NA | NA | NA | 430.098 | −1.599478 | uc011mad.2 |
| GART | 138440 | 1053.27 | −8.068135 | uc002yrx.3 | chr21: 34876238- | NA | NA | NA |
| | | | | | 34876430 | | | |
| GAS2 | 602835 | NA | NA | NA | NA | NA | NA | NA |
| GAS2L1 | 602128 | 894.436 | −5.190394 | uc010gvm.1 | chr22: 29708487- | NA | NA | NA |
| | | | | | 29708770 | | | |
| GAS8 | 605178 | 545.955 | −2.432912 | uc002fqh.2 | chr16: 90109754- | 502.768 | −2.402873 | uc002fqh.2 |
| | | | | | 90111379 | | | |
| GATA1 | 305371 | NA | NA | NA | NA | 261.925 | −5.031061 | uc004dkq.4 |
| GATS | NA | NA | NA | NA | NA | 785.879 | −10.853379 | uc003uua.4 |
| GBAS | 603004 | NA | NA | NA | NA | 206.811 | −4.605375 | uc003tre.2 |
| GBE1 | 607839 | 661.112 | −6.214096 | uc021xav.1 | chr3: 81538850- | NA | NA | NA |
| | | | | | 81539557 | | | |
| GBP3 | 600413 | NA | NA | NA | NA | 355.344 | −5.805202 | uc001dmt.3 |
| GBP5 | 611467 | 772.782 | −7.02278 | uc001dnc.3 | chr1: 89724634- | 627.735 | −5.574275 | uc001dnc.3 |
| | | | | | 89726386 | | | |
| GBP6 | 612467 | 602.591 | −5.102371 | uc001dnf.2 | chr1: 89851029- | NA | NA | NA |
| | | | | | 89853719 | | | |
| GBP7 | 612468 | NA | NA | NA | NA | NA | NA | NA |
| GCA | 607030 | 331.054 | −8.815 | uc002ucg.3 | chr2: 163216754- | NA | NA | NA |
| | | | | | 163219148 | | | |
| GCC2 | 612711 | 1537.17 | −16.631634 | uc002tec.3 | chr2: 109124087- | NA | NA | NA |
| | | | | | 109125854 | | | |
| GCH1 | 600225 | 1017.44 | −7.49047 | uc001xbh.1 | chr14: 55308724- | NA | NA | NA |
| | | | | | 55309772 | | | |
| GCLC | 606857 | 489.131 | −9.716712 | uc003pbv.1 | chr6: 53362140- | NA | NA | NA |
| | | | | | 53363553 | | | |
| GCN1L1 | NA | NA | NA | NA | NA | NA | NA | NA |
| GCNT7 | NA | 1180.81 | −8.739465 | NA | NA | 504.912 | −4.114195 | uc010zzg.1 |
| GDA | 139260 | NA | NA | NA | NA | 1001.15 | −3.608502 | uc011lse.2 |
| GDAP1 | 606598 | NA | NA | NA | NA | 329.676 | −5.818446 | uc003yah.3 |
| GDF15 | 605312 | NA | NA | NA | NA | NA | NA | NA |
| GDNF | 600837 | 686.512 | −5.458576 | uc011cpd.2 | chr5: 37812779- | NA | NA | NA |
| | | | | | 37815752 | | | |
| GDPD1 | 616317 | 512.614 | −1.780268 | uc002ixj.3 | chr17: 57350249- | NA | NA | NA |
| | | | | | 57350382 | | | |
| GDPD5 | 609632 | 381.985 | −7.312371 | uc009yue.1 | chr11: 75153809- | NA | NA | NA |
| | | | | | 75154117 | | | |
| GEMIN7 | 607419 | NA | NA | NA | NA | 692.939 | −7.349378 | uc002pap.1 |
| GEMIN8 | 300962 | NA | NA | NA | NA | NA | NA | NA |
| GEN1 | 612449 | NA | NA | NA | NA | NA | NA | NA |
| GFM1 | 606639 | 631.119 | −10.963277 | uc003fcd.3 | chr3: 158399959- | 1187.51 | −17.205622 | uc003fcd.3 |
| | | | | | 158400222 | | | |
| GGA2 | 606005 | 372.756 | −4.474012 | uc002dlq.3 | chr16: 23474863- | NA | NA | NA |
| | | | | | 23478910 | | | |
| GGT5 | 137168 | 590.948 | −6.591359 | uc002zzq.4 | chr22: 24615622- | NA | NA | NA |
| | | | | | 24616084 | | | |
| GHRL | 605353 | 808.446 | −5.806062 | uc010hdc.2 | chr3: 10327434- | NA | NA | NA |
| | | | | | 10327558 | | | |
| GID4 | NA | NA | NA | NA | NA | 500.777 | −5.296089 | uc002gsg.1 |
| GID8 | 611625 | NA | NA | NA | NA | NA | NA | NA |
| GIF | 609342 | 509.076 | −6.433731 | uc001noi.3 | chr11: 59596746- | NA | NA | NA |
| | | | | | 59596956 | | | |
| GIMAP2 | 608085 | 569.59 | −6.69738 | uc003who.3 | chr7: 150390389- | NA | NA | NA |
| | | | | | 150390728 | | | |
| GIMAP4 | 608087 | NA | NA | NA | NA | 1137.09 | −5.47148 | uc011kuu.2 |
| GINS3 | 610610 | 498.933 | −6.040669 | uc002enh.4 | chr16: 58438634- | NA | NA | NA |
| | | | | | 58440048 | | | |
| GIPC2 | NA | NA | NA | NA | NA | 761.653 | −9.047105 | uc001dik.3 |
| GIT2 | 608564 | NA | NA | NA | NA | 578.792 | −5.597539 | uc001tpq.2 |
| GJA3 | 121015 | NA | NA | NA | NA | NA | NA | NA |
| GJB3 | 603324 | NA | NA | NA | NA | 464.944 | −4.629077 | uc001bxx.3 |
| GJC1 | 608655 | NA | NA | NA | NA | NA | NA | NA |
| GJC3 | 611925 | 597.309 | −7.899391 | uc011kjd.2 | chr7: 99520892- | NA | NA | NA |
| | | | | | 99521167 | | | |
| GKAP1 | 611356 | NA | NA | NA | NA | NA | NA | NA |
| GLA | 300644 | NA | NA | NA | NA | 388.238 | −10.788676 | uc011mrj.1 |
| GLDN | 608603 | 240.079 | −4.620964 | uc002aba.3 | chr15: 51696952- | NA | NA | NA |
| | | | | | 51700209 | | | |
| GLE1 | 603371 | NA | NA | NA | NA | NA | NA | NA |
| GLG1 | 600753 | NA | NA | NA | NA | NA | NA | NA |
| GLI2 | 165230 | NA | NA | NA | NA | NA | NA | NA |
| GLI3 | 165240 | 677.321 | −6.553227 | uc011kbg.2 | chr7: 42000548- | NA | NA | NA |
| | | | | | 42003927 | | | |
| GLIPR2 | 607141 | 784.96 | −2.814864 | uc011lpj.1 | chr9: 36151033- | NA | NA | NA |
| | | | | | 36151768 | | | |
| GLIS3 | 610192 | 936.278 | −5.799423 | uc010mhf.1 | chr9: 3824128- | 807.78 | −9.246272 | uc003zhy.1 |
| | | | | | 3828271 | | | |
| GLRA2 | 305990 | NA | NA | NA | NA | 543.498 | −6.989327 | uc004cwe.4 |
| GLRA3 | 600421 | NA | NA | NA | NA | NA | NA | NA |
| GLRA4 | NA | NA | NA | NA | NA | NA | NA | NA |
| GLRB | 138492 | NA | NA | NA | NA | NA | NA | NA |
| GLRX | 600443 | NA | NA | NA | NA | NA | NA | NA |
| GLRX2 | 606820 | NA | NA | NA | NA | NA | NA | NA |
| GLRX3 | 612754 | NA | NA | NA | NA | NA | NA | NA |
| GLRX5 | 609588 | NA | NA | NA | NA | 555.405 | −7.675977 | uc001yem.1 |
| GLS | 138280 | NA | NA | NA | NA | 915.295 | −11.581377 | uc002usd.2 |
| GM2A | 613109 | 628.067 | −5.948531 | uc003ltr.4 | chr5: 150647013- | NA | NA | NA |
| | | | | | 150649953 | | | |
| GMEB1 | 604409 | NA | NA | NA | NA | NA | NA | NA |
| GMPPB | 615320 | 550.579 | −7.533205 | uc003cxk.1 | chr3: 49758932- | NA | NA | NA |
| | | | | | 49759184 | | | |
| GNB2L1 | 176981 | NA | NA | NA | NA | 828.664 | −9.456162 | uc011dhl.2 |
| GNB3 | 139130 | NA | NA | NA | NA | 237.906 | −6.473777 | uc001qrc.3 |
| GNB4 | 610863 | NA | NA | NA | NA | 636.955 | −5.936995 | uc003fjv.4 |
| GNG7 | 604430 | NA | NA | NA | NA | 1311.29 | −9.456005 | uc002lwd.2 |
| GNL1 | 143024 | NA | NA | NA | NA | 212.891 | −4.441025 | uc011dmi.2 |
| GNL3 | 608011 | NA | NA | NA | NA | 650.285 | −6.299344 | uc003dfd.3 |
| GNN | NA | NA | NA | NA | NA | 227.006 | −5.26967 | uc009zuh.2 |
| GNPTAB | 607840 | NA | NA | NA | NA | NA | NA | NA |
| GNRHR | 138850 | NA | NA | NA | NA | 853.116 | −7.358474 | uc003hdm.3 |
| GNS | 607664 | NA | NA | NA | NA | 326.351 | −8.724808 | uc001ssf.3 |
| GOLGA3 | 602581 | 1312.16 | −14.043839 | uc001ukz.1 | chr12: 133345495- | 266.169 | −2.815295 | uc001ukz.1 |
| | | | | | 133349690 | | | |
| GOLGA6B | NA | 274.284 | −8.757456 | uc010uks.1 | chr15: 72958684- | NA | NA | NA |
| | | | | | 72959738 | | | |
| GOLGA6L10 | NA | 531.124 | −11.55694 | uc021ssn.1 | chr15: 82633123- | NA | NA | NA |
| | | | | | 82635129 | | | |
| GOLGA8IP | NA | 466.089 | −6.674369 | uc001yvh.1 | chr15: 23262503- | NA | NA | NA |
| | | | | | 23262743 | | | |
| GOLPH3 | 612207 | NA | NA | NA | NA | 812.452 | −7.2485 | uc003jhp.1 |
| GOLT1B | 615078 | 275.602 | −8.368115 | uc001rez.2 | chr12: 21668642- | NA | NA | NA |
| | | | | | 21671337 | | | |
| GON4L | 610393 | 581.207 | −3.545006 | uc001fly.1 | chr1: 155719510- | NA | NA | NA |
| | | | | | 155720374 | | | |
| GORASP2 | 608693 | NA | NA | NA | NA | 1999.34 | −15.973411 | uc002ugj.3 |
| GOSR1 | 604026 | NA | NA | NA | NA | NA | NA | NA |
| GOSR2 | 604027 | NA | NA | NA | NA | 642.859 | −2.288994 | uc002iky.3 |
| GP6 | 605546 | NA | NA | NA | NA | 477.888 | −4.551622 | uc002qil.3 |
| GPALPP1 | NA | NA | NA | NA | NA | NA | NA | NA |
| GPAM | 602395 | NA | NA | NA | NA | 1003.13 | −7.103233 | uc001kzq.1 |
| GPC3 | 300037 | NA | NA | NA | NA | 1411.16 | −16.024273 | uc004exe.2 |
| GPC5 | 602446 | 851.283 | −10.241773 | uc010tif.2 | chr13: 93518693- | NA | NA | NA |
| | | | | | 93519487 | | | |
| GPC6 | 604404 | 214.994 | −3.411675 | uc001vlt.3 | chr13: 95055472- | 223.857 | 1.985351 | uc010tig.1 |
| | | | | | 95060273 | | | |
| GPCPD1 | 614124 | NA | NA | NA | NA | 370.783 | −11.2352 | uc002wmd.4 |
| GPHA2 | 609651 | NA | NA | NA | NA | NA | NA | NA |
| GPHN | 603930 | NA | NA | NA | NA | 1656.65 | −6.922271 | uc001xiw.3 |
| GPKOW | NA | NA | NA | NA | NA | NA | NA | NA |
| GPLD1 | 602515 | 758.828 | −8.948163 | uc010jpr.1 | chr6: 24447759- | 222.364 | −4.499585 | uc010jpr.1 |
| | | | | | 24448093 | | | |
| GPM6A | 601275 | NA | NA | NA | NA | 546.6 | −6.224167 | uc011ckj.2 |
| GPN3 | NA | NA | NA | NA | NA | 285.124 | −4.421335 | uc001tqr.3 |
| GPR1 | 600239 | 799.161 | −9.667094 | uc002vbl.4 | chr2: 207040042- | NA | NA | NA |
| | | | | | 207040903 | | | |
| GPR137C | NA | 757.374 | −11.828064 | uc001wzt.4 | chr14: 53101834- | NA | NA | NA |
| | | | | | 53104431 | | | |
| GPR157 | NA | NA | NA | NA | NA | NA | NA | NA |
| GPR171 | NA | NA | NA | NA | NA | 346.069 | −7.007312 | uc003eyq.4 |
| GPR19 | 602927 | 586.762 | −8.979627 | uc001rar.3 | chr12: 12813995- | NA | NA | NA |
| | | | | | 12814134 | | | |
| GPR35 | 602646 | NA | NA | NA | NA | NA | NA | NA |
| GPR37L1 | NA | 249.015 | −4.794158 | uc001gxj.3 | chr1: 202097685- | NA | NA | NA |
| | | | | | 202098634 | | | |
| GPR4 | 600551 | 903.261 | −8.124066 | uc002pcm.3 | chr19: 46093023- | NA | NA | NA |
| | | | | | 46094035 | | | |
| GPR45 | 604838 | NA | NA | NA | NA | 251.641 | −4.15004 | uc002tco.1 |
| GPR63 | 606915 | 1134.99 | −12.644913 | uc003pou.3 | chr6: 97241998- | 1096.24 | −10.97671 | uc003pou.3 |
| | | | | | 97246347 | | | |
| GPR75 | 606704 | 1327.86 | −9.686062 | uc002rxo.3 | chr2: 54080050- | NA | NA | NA |
| | | | | | 54080270 | | | |
| GPR82 | 300748 | 566.831 | −9.504369 | uc004dft.3 | chrX: 41587291- | NA | NA | NA |
| | | | | | 41589388 | | | |
| GPR84 | 606383 | 269.256 | −7.635547 | uc001sfu.3 | chr12: 54756229- | NA | NA | NA |
| | | | | | 54756444 | | | |
| GPR87 | 606379 | 330.89 | −3.48427 | uc003eyt.2 | chr3: 151011876- | 794.18 | −8.335227 | NA |
| | | | | | 151011956 | | | |
| GPRASP1 | 300417 | NA | NA | NA | NA | NA | NA | NA |
| GPRIN3 | 611241 | NA | NA | NA | NA | 1402.16 | −8.007053 | uc003hsm.1 |
| GPSM2 | 609245 | 743.058 | −3.327729 | uc010ovc.2 | chr1: 109472563- | 444.655 | −4.699684 | uc010ovc.2 |
| | | | | | 109473044 | | | |
| GPX1 | 138320 | 259.836 | −5.881273 | uc021wxw.1 | chr3: 49394609- | NA | NA | NA |
| | | | | | 49394820 | | | |
| GPX8 | NA | NA | NA | NA | NA | NA | NA | NA |
| GRAMD2 | NA | NA | NA | NA | NA | 550.368 | −4.820783 | uc010ukh.2 |
| GRAMD3 | NA | NA | NA | NA | NA | NA | NA | NA |
| GRAMD4 | 613691 | NA | NA | NA | NA | 1054.4 | −7.404716 | uc003bhx.3 |
| GRAP2 | 604518 | NA | NA | NA | NA | NA | NA | NA |
| GREB1L | NA | NA | NA | NA | NA | NA | NA | NA |
| GRHL2 | 608576 | 222.941 | −2.428788 | uc010mbt.1 | chr8: 102555788- | NA | NA | NA |
| | | | | | 102556298 | | | |
| GRIA2 | 138247 | 478.105 | −4.37695 | uc011ciu.1 | chr4: 158282726- | NA | NA | NA |
| | | | | | 158282804 | | | |
| GRIA3 | 305915 | 1736.1 | −15.383554 | uc004etq.4 | chrX: 122622564- | NA | NA | NA |
| | | | | | 122624766 | | | |
| GRIA4 | 138246 | 650.823 | −8.438075 | uc001piu.1 | chr11: 105782635- | NA | NA | NA |
| | | | | | 105784231 | | | |
| GRID1 | 610659 | NA | NA | NA | NA | NA | NA | NA |
| GRIK1 | 138245 | 707.712 | −11.248785 | uc002ynn.3 | chr21: 30909254- | NA | NA | NA |
| | | | | | 30909550 | | | |
| GRIK2 | 138244 | 1410.42 | −4.070226 | uc021zdj.1 | chr6: 102192585- | 1251.7 | −6.487024 | uc003pqn.3 |
| | | | | | 102193018 | | | |
| GRIN3A | 606650 | NA | NA | NA | NA | 627.249 | −7.840127 | uc004bbq.1 |
| GRM7 | 604101 | NA | NA | NA | NA | 743.9 | −10.867232 | uc003bqm.2 |
| GRM8 | 601116 | 338.353 | −6.836101 | uc003vlr.2 | chr7: 126078652- | NA | NA | NA |
| | | | | | 126079172 | | | |
| GRP | 137260 | 818.068 | −11.220853 | uc002lhu.3 | chr18: 56897690- | 809.861 | −11.220853 | uc002lhu.3 |
| | | | | | 56898002 | | | |
| GRPEL2 | NA | 644.539 | −4.553067 | uc003lqj.3 | chr5: 148730846- | 832.479 | −4.141222 | uc003lqj.3 |
| | | | | | 148734146 | | | |
| GRSF1 | 604851 | NA | NA | NA | NA | 1111.23 | −9.644448 | uc011caz.1 |
| GRTP1 | NA | 1033.47 | −6.486554 | uc001vtn.3 | chr13: 113978505- | NA | NA | NA |
| | | | | | 113978802 | | | |
| GRXCR1 | 613283 | 1169.06 | −16.082854 | uc003gwt.3 | chr4: 43032558- | NA | NA | NA |
| | | | | | 43032675 | | | |
| GSDMB | 611221 | NA | NA | NA | NA | 965.377 | −6.703814 | uc010cwi.3 |
| GSDMC | 608384 | NA | NA | NA | NA | 567.414 | −6.215394 | uc003ysr.3 |
| GSR | 138300 | NA | NA | NA | NA | 1640.99 | −15.053228 | uc003xih.2 |
| GSTA1 | 138359 | NA | NA | NA | NA | NA | NA | NA |
| GSTM2 | 138380 | 818.093 | −6.737641 | uc010ovt.2 | chr1: 110225829- | NA | NA | NA |
| | | | | | 110226619 | | | |
| GSTM3 | 138390 | NA | NA | NA | NA | 746.462 | −6.673209 | uc001dyo.2 |
| GSTM4 | 138333 | 557.652 | −10.354395 | uc009wfi.2 | chr1: 110199547- | NA | NA | NA |
| | | | | | 110199958 | | | |
| GSTM5 | 138385 | NA | NA | NA | NA | NA | NA | NA |
| GSTO2 | 612314 | NA | NA | NA | NA | 640.889 | −3.796864 | uc001kyb.3 |
| GTDC1 | 610165 | 666.982 | −10.455262 | uc002tvo.3 | chr2: 144703581- | 549.625 | −6.054358 | uc002tvo.3 |
| | | | | | 144704702 | | | |
| GTF2A2 | 600519 | NA | NA | NA | NA | NA | NA | NA |
| GTF2E2 | 189964 | 603.028 | −6.011422 | uc003xig.3 | chr8: 30436031- | NA | NA | NA |
| | | | | | 30436437 | | | |
| GTF2H2C | NA | 548.867 | −10.493739 | uc003jwx.4 | chr5: 68888026- | NA | NA | NA |
| | | | | | 68888729 | | | |
| GTF2I | 601679 | 906.229 | −10.335831 | uc003uat.3 | chr7: 74131276- | 1268.65 | −10.088877 | uc003uat.3 |
| | | | | | 74131386 | | | |
| GTF2IRD2B | 608900 | NA | NA | NA | NA | 422.827 | −4.4703 | uc003ubs.4 |
| GTF3C3 | 604888 | NA | NA | NA | NA | 205.683 | −6.119731 | uc002uts.3 |
| GTF3C6 | 611784 | 456.111 | −6.351604 | uc003pum.3 | chr6: 111288994- | NA | NA | NA |
| | | | | | 111289091 | | | |
| GTPBP2 | 607434 | 869.923 | −5.418562 | uc010jyv.3 | chr6: 43588218- | NA | NA | NA |
| | | | | | 43589350 | | | |
| GTPBP4 | NA | 1288.89 | −10.044339 | uc001ift.3 | chr10: 1063168- | NA | NA | NA |
| | | | | | 1063708 | | | |
| GTPBP6 | 300124 | 635.265 | −7.463472 | uc004cpe.1 | chrX: 221426- | NA | NA | NA |
| | | | | | 221740 | | | |
| GTSF1 | NA | NA | NA | NA | NA | 1197.86 | −11.818668 | uc001sgb.3 |
| GUCA1A | 600364 | 1122.55 | −13.049034 | uc003orx.3 | chr6: 42147142- | NA | NA | NA |
| | | | | | 42147794 | | | |
| GUCY1A3 | 139396 | 969.772 | −7.017034 | uc003iov.3 | chr4: 156651384- | NA | NA | NA |
| | | | | | 156658214 | | | |
| GUF1 | NA | 606.411 | −7.451648 | uc003gww.4 | chr4: 44700699- | 385.801 | −4.93645 | uc003gww.4 |
| | | | | | 44702697 | | | |
| GULP1 | 608165 | 656.015 | −16.476089 | uc002uqd.3 | chr2: 189458689- | NA | NA | NA |
| | | | | | 189460652 | | | |
| GUSB | 611499 | NA | NA | NA | NA | NA | NA | NA |
| GUSBP1 | NA | 1001.99 | −8.432764 | uc003jgf.3 | chr5: 21470807- | NA | NA | NA |
| | | | | | 21471090 | | | |
| GYG1 | 603942 | 302.975 | −4.089288 | uc011bnp.2 | chr3: 148741953- | NA | NA | NA |
| | | | | | 148742059 | | | |
| GYG2 | 300198 | 661.412 | −6.137429 | uc004cqs.1 | chrX: 2799255- | NA | NA | NA |
| | | | | | 2800861 | | | |
| GYG2P1 | NA | NA | NA | NA | NA | NA | NA | NA |
| GYPE | 138590 | NA | NA | NA | NA | NA | NA | NA |
| GZF1 | 613842 | 560.696 | −7.635321 | uc002wsy.3 | chr20: 23351079- | NA | NA | NA |
| | | | | | 23353683 | | | |
| GZMK | 600784 | NA | NA | NA | NA | 722.612 | −8.298611 | uc003jpl.1 |
| H1F0 | 142708 | NA | NA | NA | NA | NA | NA | NA |
| H1FX | 602785 | 773.185 | −6.755107 | uc003elx.3 | chr3: 129033614- | NA | NA | NA |
| | | | | | 129034103 | | | |
| H2AFY | 610054 | NA | NA | NA | NA | 235.151 | −5.804303 | uc003lam.1 |
| H2AFZ | 142763 | 250.618 | −5.622688 | uc003hvo.1 | chr4: 100869244- | 714.97 | −7.821444 | uc003hvn.1 |
| | | | | | 100869618 | | | |
| H3F3C | 616134 | 423.505 | −6.710187 | uc001rkr.3 | chr12: 31944119- | NA | NA | NA |
| | | | | | 31944692 | | | |
| HACD3 | 615940 | 248.654 | −4.06825 | uc002apc.3 | chr15: 65868718- | NA | NA | NA |
| | | | | | 65870693 | | | |
| HACD4 | 615941 | NA | NA | NA | NA | 225.269 | −8.49867 | uc010miq.2 |
| HADHA | 600890 | NA | NA | NA | NA | 280.368 | −6.706005 | uc010ykt.1 |
| HAGH | 138760 | 400.755 | −4.620363 | uc002cmz.3 | chr16: 1859104- | NA | NA | NA |
| | | | | | 1859283 | | | |
| HAO1 | 605023 | 969.455 | −3.749254 | uc002wmw.1 | chr20: 7863631- | 836.527 | −9.767606 | uc002wmw.1 |
| | | | | | 7864239 | | | |
| HAP1 | 600947 | 1641.66 | −12.465227 | uc002hxm.1 | chr17: 39878891- | NA | NA | NA |
| | | | | | 39880952 | | | |
| HARS | 142810 | NA | NA | NA | NA | 329.968 | −4.314917 | uc003lgu.3 |
| HARS2 | 600783 | 495.555 | −5.163575 | uc003lgx.3 | chr5: 140078138- | NA | NA | NA |
| | | | | | 140078890 | | | |
| HAS2 | 601636 | NA | NA | NA | NA | NA | NA | NA |
| HAS3 | 602428 | NA | NA | NA | NA | NA | NA | NA |
| HAT1 | 603053 | 919.158 | −12.130504 | uc010fqi.2 | chr2: 172844358- | NA | NA | NA |
| | | | | | 172845768 | | | |
| HAUS2 | 613429 | NA | NA | NA | NA | 555.61 | −5.128775 | uc001zqe.3 |
| HAUS3 | 613430 | 563.613 | −5.861762 | uc011bvj.1 | chr4: 2229191- | NA | NA | NA |
| | | | | | 2230137 | | | |
| HAUS4 | 613431 | NA | NA | NA | NA | 797.1 | −9.780509 | uc001wht.3 |
| HAUS8 | 613434 | 1011.5 | −16.975417 | uc002nfe.3 | chr19: 17160571- | NA | NA | NA |
| | | | | | 17160682 | | | |
| HAVCR1 | 606518 | NA | NA | NA | NA | 476.708 | −5.594051 | uc011ddl.1 |
| HAVCR2 | 606652 | NA | NA | NA | NA | 1451.7 | −16.894046 | uc003lwk.2 |
| HBB | 141900 | 272.765 | −4.783462 | uc001mae.1 | chr11: 5246696- | NA | NA | NA |
| | | | | | 5246827 | | | |
| HBG1 | 142200 | NA | NA | NA | NA | 1292.27 | −8.499943 | uc001mah.1 |
| HBG2 | 142250 | NA | NA | NA | NA | 468.41 | −4.294229 | uc001mai.1 |
| HBP1 | NA | 707.129 | −3.232032 | uc011klv.2 | chr7: 106841877- | NA | NA | NA |
| | | | | | 106842974 | | | |
| HCCS | 300056 | NA | NA | NA | NA | NA | NA | NA |
| HCFC2 | 607926 | 567.08 | −8.713057 | uc001tkj.4 | chr12: 104497052- | NA | NA | NA |
| | | | | | 104500304 | | | |
| HCN1 | 602780 | NA | NA | NA | NA | NA | NA | NA |
| HCRTR1 | 602392 | NA | NA | NA | NA | NA | NA | NA |
| HDAC1 | 601241 | 1114.36 | −10.665396 | uc001bvb.1 | chr1: 32798646- | NA | NA | NA |
| | | | | | 32799224 | | | |
| HDAC11 | 607226 | 425.649 | −3.397031 | uc010heb.3 | chr3: 13545978- | 499.568 | −7.300275 | uc003bxy.3 |
| | | | | | 13547924 | | | |
| HDAC5 | 605315 | 468.205 | −5.539813 | uc002ifd.1 | chr17: 42154121- | NA | NA | NA |
| | | | | | 42155743 | | | |
| HDDC2 | NA | NA | NA | NA | NA | 221.264 | −5.985994 | uc003qaa.1 |
| HDHD2 | NA | NA | NA | NA | NA | NA | NA | NA |
| HDLBP | 142695 | NA | NA | NA | NA | NA | NA | NA |
| HDX | NA | 479.116 | −5.551374 | uc004eek.2 | chrX: 83572882- | 267.539 | −5.733285 | uc004eek.2 |
| | | | | | 83576896 | | | |
| HEATR1 | NA | NA | NA | NA | NA | NA | NA | NA |
| HEATR5A | NA | NA | NA | NA | NA | 743.345 | −5.923901 | uc010tpk.1 |
| HEATR5B | NA | 991.452 | −13.547208 | uc010ezy.1 | chr2: 37208153- | NA | NA | NA |
| | | | | | 37208632 | | | |
| HEATR9 | NA | 444.842 | −5.591052 | uc002hke.1 | chr17: 34181960- | 296.258 | −3.56388 | uc002hke.1 |
| | | | | | 34182066 | | | |
| HECA | 607977 | NA | NA | NA | NA | 2242.09 | −21.730613 | uc003qin.3 |
| HECTD1 | NA | NA | NA | NA | NA | NA | NA | NA |
| HECTD2 | NA | 300.935 | −8.212902 | uc001khk.2 | chr10: 93221966- | 504.901 | −7.264428 | uc001khk.2 |
| | | | | | 93222967 | | | |
| HECTD3 | NA | NA | NA | NA | NA | NA | NA | NA |
| HECTD4 | NA | 943.174 | −9.121611 | uc001ttt.3 | chr12: 112687361- | NA | NA | NA |
| | | | | | 112687931 | | | |
| HELLS | 603946 | NA | NA | NA | NA | NA | NA | NA |
| HELQ | 606769 | 1409.43 | −10.815639 | uc003hom.3 | chr4: 84328499- | NA | NA | NA |
| | | | | | 84328605 | | | |
| HELZ | 606699 | NA | NA | NA | NA | 640.27 | −4.137071 | uc002jfx.4 |
| HEPACAM2 | 614133 | NA | NA | NA | NA | NA | NA | NA |
| HEPH | 300167 | NA | NA | NA | NA | 1089.47 | −7.263028 | uc011moz.2 |
| HERC1 | 605109 | 639.61 | −9.796354 | uc002amp.3 | chr15: 63900817- | NA | NA | NA |
| | | | | | 63901279 | | | |
| HERC2 | 605837 | 530.16 | −5.038391 | uc001zbk.1 | chr15: 28435347- | NA | NA | NA |
| | | | | | 28436077 | | | |
| HERC4 | 609248 | 454.693 | −6.571994 | uc021prr.1 | chr10: 69724549- | 722.99 | −7.705469 | uc009xpq.3 |
| | | | | | 69726415 | | | |
| HEXB | 606873 | 258.939 | −7.773584 | uc003kdd.3 | chr5: 74017001- | NA | NA | NA |
| | | | | | 74017113 | | | |
| HEXDC | NA | NA | NA | NA | NA | 255.801 | −5.0142 | uc010wvn.1 |
| HEXIM1 | 607328 | NA | NA | NA | NA | 346.194 | −7.93743 | uc002iig.3 |
| HHIP | 606178 | 213.402 | −4.052466 | uc003ljr.2 | chr4: 145581123- | 943.94 | −7.878725 | uc003ijs.2 |
| | | | | | 145581180 | | | |
| HIATL1 | NA | NA | NA | NA | NA | NA | NA | NA |
| HIGD1A | NA | NA | NA | NA | NA | 1625.03 | −10.161489 | uc010hid.3 |
| HILPDA | NA | NA | NA | NA | NA | NA | NA | NA |
| HIP1R | 605613 | NA | NA | NA | NA | NA | NA | NA |
| HIPK1 | 608003 | 426.473 | −4.37522 | uc001eem.3 | chr1: 114516135- | NA | NA | NA |
| | | | | | 114520491 | | | |
| HIST1H2AB | 602795 | 270.781 | −4.116528 | NA | NA | 643.996 | −4.373225 | uc003nft.1 |
| HIVEP1 | 194540 | 906.098 | −12.092224 | uc003nac.3 | chr6: 12164695- | 999.223 | −6.385728 | uc003nac.3 |
| | | | | | 12165232 | | | |
| HIVEP2 | 143054 | 1291.41 | −8.577927 | uc003qjd.3 | chr6: 143072604- | NA | NA | NA |
| | | | | | 143074243 | | | |
| HJURP | 612667 | 1496.48 | −9.264957 | uc002VVg.3 | chr2: 234745486- | NA | NA | NA |
| | | | | | 234746222 | | | |
| HKR1 | 165250 | 510.586 | −12.129491 | uc002ofy.3 | chr19: 37854678- | NA | NA | NA |
| | | | | | 37855357 | | | |
| HLF | 142385 | 980.928 | −7.016767 | uc002iug.1 | chr17: 53398241- | NA | NA | NA |
| | | | | | 53402426 | | | |
| HLTF | 603257 | NA | NA | NA | NA | 591.192 | −4.146581 | uc003ewq.1 |
| HMBOX1 | NA | NA | NA | NA | NA | NA | NA | NA |
| HMBS | 609806 | NA | NA | NA | NA | NA | NA | NA |
| HMCES | NA | 1051.82 | −8.505098 | uc003elt.3 | chr3: 129023669- | 788.633 | −8.431328 | uc003elt.3 |
| | | | | | 129024135 | | | |
| HMG20A | 605534 | NA | NA | NA | NA | NA | NA | NA |
| HMGA2 | 600698 | NA | NA | NA | NA | 257.017 | −4.072378 | uc001ssu.1 |
| HMGB1 | 163905 | 1213.58 | −6.735242 | uc001usv.3 | chr13: 31032879- | 678.647 | −4.412205 | uc001usv.3 |
| | | | | | 31035670 | | | |
| HMGB2 | 163906 | NA | NA | NA | NA | 533.776 | −5.458538 | uc011ckc.1 |
| HMGCLL1 | NA | 688.848 | −9.096557 | uc003pcn.3 | chr6: 55299171- | NA | NA | NA |
| | | | | | 55300459 | | | |
| HMGN1 | 163920 | NA | NA | NA | NA | NA | NA | NA |
| HMGN2 | 163910 | NA | NA | NA | NA | 869.866 | −9.501937 | uc001bmp.4 |
| HMGN3 | 604502 | NA | NA | NA | NA | NA | NA | NA |
| HMGN4 | NA | NA | NA | NA | NA | 777.836 | −11.107483 | uc003nig.3 |
| HMGXB4 | 604702 | 803.146 | −8.289808 | uc003ank.3 | chr22: 35689645- | 466.37 | −9.592067 | uc003ank.3 |
| | | | | | 35691800 | | | |
| HMHA1 | 601155 | NA | NA | NA | NA | 534.978 | −4.667753 | uc002lra.1 |
| HMMR | 600936 | 1427.08 | −15.72479 | uc003lzf.3 | chr5: 162918171- | 1778.43 | −23.870699 | uc003lzf.3 |
| | | | | | 162918953 | | | |
| HN1 | NA | 1087.27 | −8.488559 | uc002jnb.1 | chr17: 73131344- | 1051.92 | −7.228869 | uc002jmz.1 |
| | | | | | 73132096 | | | |
| HNF1B | 189907 | 712.161 | −7.367194 | uc021tvu.1 | chr17: 36046434- | NA | NA | NA |
| | | | | | 36047374 | | | |
| HNRNPA0 | 609409 | NA | NA | NA | NA | NA | NA | NA |
| HNRNPC | 164020 | NA | NA | NA | NA | 476.725 | −6.960962 | uc001vzw.3 |
| HNRNPD | 601324 | 1467.23 | −19.1491 | uc010ijr.1 | chr4: 83275908- | 556.923 | −8.355371 | uc010ijr.1 |
| | | | | | 83276486 | | | |
| HNRNPH3 | 602324 | 251.297 | −5.760401 | uc001jnw.4 | chr10: 70101812- | NA | NA | NA |
| | | | | | 70102953 | | | |
| HNRNPK | 600712 | 621.666 | −7.169206 | uc011lsw.2 | chr9: 86582998- | 542.973 | −5.639248 | uc004and.4 |
| | | | | | 86584321 | | | |
| HNRNPL | 603083 | NA | NA | NA | NA | NA | NA | NA |
| HNRNPLL | 611208 | 805.374 | −8.483596 | uc002rqv.3 | chr2: 38790328- | NA | NA | NA |
| | | | | | 38791323 | | | |
| HNRNPM | 160994 | 656.076 | −9.916232 | uc010dwd.3 | chr19: 8553739- | 1351.81 | −12.245255 | uc010dwd.3 |
| | | | | | 8554002 | | | |
| HOMER3 | 604800 | NA | NA | NA | NA | 216.706 | −4.084102 | uc010eby.2 |
| HOOK3 | 607825 | 1258.55 | −9.324166 | uc010lxq.1 | chr8: 42852895- | 1029.17 | −10.301969 | uc010lxq.1 |
| | | | | | 42853299 | | | |
| HORMAD1 | 609824 | 419.1 | −6.446522 | uc001evk.2 | chr1: 150670535- | NA | NA | NA |
| | | | | | 150671129 | | | |
| HORMAD2 | NA | NA | NA | NA | NA | NA | NA | NA |
| HOXA13 | 142959 | NA | NA | NA | NA | 616.608 | −7.031625 | uc003szb.1 |
| HOXA2 | 604685 | NA | NA | NA | NA | NA | NA | NA |
| HOXA4 | 142953 | 1139.01 | −6.8996 | uc003sym.4 | chr7: 27168126- | NA | NA | NA |
| | | | | | 27168843 | | | |
| HOXB2 | 142967 | 339.441 | −4.830146 | uc002inm.3 | chr17: 46620019- | NA | NA | NA |
| | | | | | 46620429 | | | |
| HOXC11 | 605559 | 847.792 | −10.338666 | uc001sem.3 | chr12: 54369198- | NA | NA | NA |
| | | | | | 54369667 | | | |
| HOXD10 | 142984 | NA | NA | NA | NA | 1345.05 | −7.539481 | uc002ukj.3 |
| HOXD3 | 142980 | 742.148 | −7.656091 | uc002ukt.1 | chr2: 177037003- | NA | NA | NA |
| | | | | | 177037826 | | | |
| HOXD8 | 142985 | NA | NA | NA | NA | NA | NA | NA |
| HOXD9 | 142982 | NA | NA | NA | NA | 1509.47 | −11.270513 | uc010zex.2 |
| HPGD | 601688 | 594.211 | −4.666856 | uc011ckh.1 | chr4: 175411328- | NA | NA | NA |
| | | | | | 175413106 | | | |
| HPGDS | 602598 | 914.11 | −9.605569 | uc003hte.1 | chr4: 95219707- | NA | NA | NA |
| | | | | | 95220630 | | | |
| HPRT1 | 308000 | NA | NA | NA | NA | 686.904 | −10.346143 | uc004exl.4 |
| HPS3 | 606118 | 650.977 | −1.294873 | uc003ewu.1 | chr3: 148890010- | NA | NA | NA |
| | | | | | 148891305 | | | |
| HRAS | 190020 | 351.437 | −4.164841 | uc010qvw.2 | chr11: 532242- | NA | NA | NA |
| | | | | | 532755 | | | |
| HS2ST1 | 604844 | NA | NA | NA | NA | NA | NA | NA |
| HS3ST3A1 | 604057 | 508.11 | −4.596967 | uc002gob.1 | chr17: 13399006- | NA | NA | NA |
| | | | | | 13399513 | | | |
| HS3ST3B1 | 604058 | 1141.69 | −10.536595 | uc002goh.1 | chr17: 14248964- | 604.67 | −6.471949 | uc002goh.1 |
| | | | | | 14249492 | | | |
| HSD17B12 | 609574 | NA | NA | NA | NA | NA | NA | NA |
| HSD17B4 | 601860 | NA | NA | NA | NA | 926.587 | −8.609898 | uc003ksj.3 |
| HSD17B7P2 | NA | 464.596 | −2.263132 | uc010qew.1 | chr10: 38647460- | 539.713 | −5.675098 | uc010qew.1 |
| | | | | | 38647980 | | | |
| HSF2 | 140581 | 773.016 | −8.970337 | uc003pyt.4 | chr6: 122741380- | 739.878 | −7.713677 | uc003pyu.2 |
| | | | | | 122741555 | | | |
| HSF4 | 602438 | 365.664 | −4.470918 | uc002erl.2 | chr16: 67203689- | NA | NA | NA |
| | | | | | 67203848 | | | |
| HSF5 | NA | NA | NA | NA | NA | 411.279 | −4.131464 | uc002iwi.1 |
| HSFX2 | NA | NA | NA | NA | NA | NA | NA | NA |
| HSP90AA1 | 140571 | NA | NA | NA | NA | 1518.57 | −9.438667 | uc001yku.4 |
| HSPA12A | 610701 | NA | NA | NA | NA | 1362.33 | −11.903135 | uc001lct.3 |
| HSPA1A | 140550 | NA | NA | NA | NA | 599.961 | −8.746472 | uc003nxj.3 |
| HSPA1B | 603012 | 1842.57 | −8.280465 | uc003nxk.2 | chr6: 31797654- | 832.883 | −5.944426 | uc003nxk.2 |
| | | | | | 31798031 | | | |
| HSPA2 | 140560 | 1754.95 | −11.056129 | uc001xhk.4 | chr14: 65009488- | NA | NA | NA |
| | | | | | 65009954 | | | |
| HSPA4L | NA | NA | NA | NA | NA | NA | NA | NA |
| HSPB1 | 602195 | 1156.39 | −9.309635 | uc003uew.3 | chr7: 75933491- | NA | NA | NA |
| | | | | | 75933614 | | | |
| HSPBAP1 | 608263 | NA | NA | NA | NA | NA | NA | NA |
| HSPG2 | 142461 | 601.618 | −5.105197 | uc001bfi.3 | chr1: 22148737- | NA | NA | NA |
| | | | | | 22149808 | | | |
| HSPH1 | 610703 | 769.634 | −9.306382 | uc001utj.3 | chr13: 31710763- | 369.43 | −5.293671 | uc010aax.1 |
| | | | | | 31711454 | | | |
| HTATIP2 | 605628 | NA | NA | NA | NA | 492.458 | −8.502708 | uc009yia.1 |
| HTN1 | 142701 | NA | NA | NA | NA | 486.373 | −6.698963 | uc003hex.3 |
| HTR1D | 182133 | 1020.86 | −10.717626 | uc001bgn.3 | chr1: 23518388- | NA | NA | NA |
| | | | | | 23519578 | | | |
| HTR2B | 601122 | NA | NA | NA | NA | NA | NA | NA |
| HTR3C | 610121 | NA | NA | NA | NA | 674.111 | −7.11863 | uc003fmk.3 |
| HTR3D | 610122 | 245.586 | −6.90591 | uc003fmj.3 | chr3: 183756764- | 578.275 | −5.201113 | uc003fmj.3 |
| | | | | | 183757157 | | | |
| HTR4 | 602164 | NA | NA | NA | NA | 690.386 | −6.910405 | uc021yfg.1 |
| HTRA4 | 610700 | 744.875 | −7.614987 | uc003xmj.3 | chr8: 38845618- | NA | NA | NA |
| | | | | | 38846181 | | | |
| HUS1B | 609713 | NA | NA | NA | NA | 985.179 | −6.951259 | uc003mtg.3 |
| HYAL2 | 603551 | NA | NA | NA | NA | NA | NA | NA |
| HYKK | 614681 | 482.76 | −8.99264 | uc002bdt.3 | chr15: 78819926- | NA | NA | NA |
| | | | | | 78820606 | | | |
| HYPK | 612784 | 572.273 | −5.199055 | uc001ztg.1 | chr15: 44093779- | NA | NA | NA |
| | | | | | 44095241 | | | |
| IAH1 | NA | 444.823 | −7.142982 | uc002qzr.3 | chr2: 9628459- | NA | NA | NA |
| | | | | | 9628591 | | | |
| IAPP | 147940 | NA | NA | NA | NA | 541.128 | −10.861205 | uc001rev.3 |
| ICA1L | NA | 544.74 | −5.730053 | uc002uzj.3 | chr2: 203684329- | NA | NA | NA |
| | | | | | 203684417 | | | |
| ICAM1 | 147840 | NA | NA | NA | NA | 437.352 | −8.241263 | uc002mnq.2 |
| ICAM5 | 601852 | NA | NA | NA | NA | NA | NA | NA |
| ICK | 612325 | 313.51 | −5.320226 | uc003pbh.2 | chr6: 52866098- | 609.151 | −5.569369 | uc003pbj.3 |
| | | | | | 52869946 | | | |
| ICMT | 605851 | 1217.77 | −9.404387 | uc001amk.3 | chr1: 6281253- | NA | NA | NA |
| | | | | | 6285139 | | | |
| ICT1 | 603000 | 593.38 | −5.846009 | uc002jmm.3 | chr17: 73017101- | NA | NA | NA |
| | | | | | 73017356 | | | |
| ID2 | 600386 | 734.245 | −8.552103 | uc002qza.3 | chr2: 8823815- | NA | NA | NA |
| | | | | | 8824583 | | | |
| IDH3A | 601149 | NA | NA | NA | NA | 704.526 | −6.744577 | uc002bdd.3 |
| IDO2 | 612129 | 729.512 | −5.46729 | uc010lwy.1 | chr8: 39873122- | NA | NA | NA |
| | | | | | 39873910 | | | |
| IER3IP1 | 609382 | NA | NA | NA | NA | 929.077 | −6.788403 | uc002lcu.3 |
| IFI16 | 147586 | NA | NA | NA | NA | 655.267 | −6.442974 | uc001ftg.3 |
| IFI35 | 600735 | NA | NA | NA | NA | NA | NA | NA |
| IFI6 | 147572 | NA | NA | NA | NA | NA | NA | NA |
| IFIT3 | 604650 | NA | NA | NA | NA | NA | NA | NA |
| IFIT5 | 616135 | NA | NA | NA | NA | NA | NA | NA |
| IFNL1 | 607403 | NA | NA | NA | NA | 659.835 | −4.809185 | uc002okv.3 |
| IFRD1 | 603502 | NA | NA | NA | NA | 1213.34 | −8.133929 | uc003vgh.3 |
| IFT172 | 607386 | NA | NA | NA | NA | 511.097 | −3.793893 | uc002rkv.3 |
| IFT43 | 614068 | NA | NA | NA | NA | 304.222 | −6.551024 | uc001xsg.2 |
| IFT52 | NA | 1394.81 | −15.233894 | uc002xkw.3 | chr20: 42275624- | NA | NA | NA |
| | | | | | 42275862 | | | |
| IFT57 | 606621 | 2118.27 | −26.269368 | uc021xcc.1 | chr3: 107879659- | NA | NA | NA |
| | | | | | 107881323 | | | |
| IFT81 | 605489 | 795.366 | −6.473521 | uc001tqg.3 | chr12: 110605784- | NA | NA | NA |
| | | | | | 110606250 | | | |
| IFT88 | 600595 | NA | NA | NA | NA | NA | NA | NA |
| IGFBP2 | 146731 | NA | NA | NA | NA | 257.509 | −7.100463 | uc010zju.2 |
| IGFBP3 | 146732 | 689.45 | −9.405339 | uc003tnr.3 | chr7: 45951844- | NA | NA | NA |
| | | | | | 45953433 | | | |
| IGFBP7 | 602867 | 439.722 | −7.743469 | uc003hcn.3 | chr4: 57897237- | NA | NA | NA |
| | | | | | 57897482 | | | |
| IGIP | NA | 477.035 | −5.629777 | uc003lfb.1 | chr5: 139508224- | NA | NA | NA |
| | | | | | 139508391 | | | |
| IGSF1 | 300137 | NA | NA | NA | NA | 505.509 | −5.131939 | uc004ewd.3 |
| IGSF10 | NA | 205.264 | −4.147118 | uc011bob.2 | chr3: 151153778- | NA | NA | NA |
| | | | | | 151154476 | | | |
| IGSF21 | NA | 735.691 | −7.147229 | uc001bau.2 | chr1: 18704821- | NA | NA | NA |
| | | | | | 18704977 | | | |
| IGSF22 | NA | 304.693 | −4.242692 | uc009yht.2 | chr11: 18725852- | NA | NA | NA |
| | | | | | 18726014 | | | |
| IHH | 600726 | 484.867 | −4.852798 | uc002vjo.2 | chr2: 219919142- | NA | NA | NA |
| | | | | | 219919928 | | | |
| IKBKB | 603258 | 2037.39 | −2.897924 | uc003xov.3 | chr8: 42174561- | NA | NA | NA |
| | | | | | 42174729 | | | |
| IKZF1 | 603023 | NA | NA | NA | NA | 1074.94 | −10.566495 | uc003tow.4 |
| IL12A | 161560 | 547.729 | −8.923444 | uc003fcx.3 | chr3: 159713347- | NA | NA | NA |
| | | | | | 159713806 | | | |
| IL13 | 147683 | NA | NA | NA | NA | NA | NA | NA |
| IL13RA1 | 300119 | 1695.68 | −7.215737 | uc004eqr.1 | chrX: 117895265- | 1382.31 | −15.951354 | uc004eqr.1 |
| | | | | | 117895477 | | | |
| IL15 | 600554 | 1073.81 | −13.6481 | uc003iis.3 | chr4: 142654002- | NA | NA | NA |
| | | | | | 142655140 | | | |
| IL16 | 603035 | NA | NA | NA | NA | 892.579 | −6.393712 | uc010unp.2 |
| IL17RD | 606807 | NA | NA | NA | NA | 246.979 | −5.224909 | uc003dik.3 |
| IL18R1 | 604494 | NA | NA | NA | NA | NA | NA | NA |
| IL1A | 147760 | 828.534 | −10.851862 | uc002tig.3 | chr2: 113531492- | 394.27 | −6.194173 | uc002tig.3 |
| | | | | | 113532643 | | | |
| IL1B | 147720 | 1118.38 | −5.416822 | uc002tih.1 | chr2: 113587337- | NA | NA | NA |
| | | | | | 113587937 | | | |
| IL1RAP | 602626 | NA | NA | NA | NA | 486.166 | −10.011083 | uc003fsm.2 |
| IL2 | 147680 | NA | NA | NA | NA | NA | NA | NA |
| IL20RA | 605620 | NA | NA | NA | NA | NA | NA | NA |
| IL24 | 604136 | NA | NA | NA | NA | NA | NA | NA |
| IL26 | 605679 | 479.82 | −6.622714 | uc001stx.1 | chr12: 68595129- | NA | NA | NA |
| | | | | | 68595624 | | | |
| IL3 | 147740 | NA | NA | NA | NA | 608.337 | −5.377847 | uc003kwe.1 |
| IL36G | 605542 | 483.916 | −8.722053 | uc002tio.1 | chr2: 113742627- | NA | NA | NA |
| | | | | | 113743227 | | | |
| IL5RA | 147851 | NA | NA | NA | NA | 238.596 | −4.879666 | uc010hbt.2 |
| IL6 | 147620 | NA | NA | NA | NA | NA | NA | NA |
| IL6ST | 600694 | NA | NA | NA | NA | 592.065 | −5.172253 | uc010iwd.3 |
| IL9 | 146931 | NA | NA | NA | NA | 689.875 | −9.209554 | uc003lbb.1 |
| ILDR1 | 609739 | 862.852 | −4.758592 | uc003eeq.3 | chr3: 121706170- | NA | NA | NA |
| | | | | | 121707213 | | | |
| ILDR2 | NA | NA | NA | NA | NA | NA | NA | NA |
| ILF3 | 603182 | NA | NA | NA | NA | 493.527 | −7.045511 | uc002mpn.3 |
| ILKAP | NA | NA | NA | NA | NA | 1083.23 | −9.904259 | uc002vxv.3 |
| ILVBL | 605770 | 382.258 | −4.551745 | uc002nam.3 | chr19: 15225785- | NA | NA | NA |
| | | | | | 15226062 | | | |
| IMPA2 | 605922 | 1066.73 | −11.926636 | uc010dlb.2 | chr18: 12017831- | NA | NA | NA |
| | | | | | 12018284 | | | |
| IMPACT | 615319 | NA | NA | NA | NA | NA | NA | NA |
| IMPDH2 | 146691 | 444.588 | −5.75435 | uc003cvt.3 | chr3: 49061762- | NA | NA | NA |
| | | | | | 49061815 | | | |
| IMPG2 | 607056 | NA | NA | NA | NA | NA | NA | NA |
| INCENP | 604411 | NA | NA | NA | NA | NA | NA | NA |
| ING1 | 601566 | NA | NA | NA | NA | 328.37 | −7.161504 | uc001vrg.3 |
| ING2 | 604215 | NA | NA | NA | NA | 994.176 | −9.182658 | uc003ivs.1 |
| ING3 | 607493 | 697.851 | −14.639805 | uc003vjm.1 | chr7: 120596226- | NA | NA | NA |
| | | | | | 120597034 | | | |
| INHBC | 601233 | NA | NA | NA | NA | 286.751 | −5.736782 | uc001snv.1 |
| INO80C | NA | 708.486 | −7.355986 | uc002kyx.4 | chr18: 33048291- | NA | NA | NA |
| | | | | | 33048574 | | | |
| INO80D | NA | NA | NA | NA | NA | NA | NA | NA |
| INPP4A | 600916 | NA | NA | NA | NA | NA | NA | NA |
| INPP4B | 607494 | NA | NA | NA | NA | 759.997 | −6.376647 | uc003iiw.4 |
| INPP5J | 606481 | NA | NA | NA | NA | NA | NA | NA |
| INPP5K | 607875 | NA | NA | NA | NA | 204.174 | −3.241847 | uc010cjs.2 |
| INSL5 | 606413 | NA | NA | NA | NA | NA | NA | NA |
| INSM2 | 614027 | 733.255 | −3.508026 | uc001wth.1 | chr14: 36005160- | 942.031 | −6.263729 | uc001wth.1 |
| | | | | | 36006260 | | | |
| INSR | 147670 | 343.791 | −6.159392 | Uc002mgf.3 | chr19: 7151995- | 290.506 | −5.934216 | uc002mgf.3 |
| | | | | | 7152708 | | | |
| INSRR | 147671 | NA | NA | NA | NA | 818.255 | −9.798998 | uc009wsj.2 |
| INTS10 | 611353 | 238.989 | −6.051403 | uc003wzj.3 | chr8: 19709317- | NA | NA | NA |
| | | | | | 19709586 | | | |
| INTS12 | 611355 | 515.313 | −6.330751 | uc003hxw.3 | chr4: 106603785- | NA | NA | NA |
| | | | | | 106603889 | | | |
| INTS3 | 611347 | NA | NA | NA | NA | NA | NA | NA |
| INTS5 | 611349 | NA | NA | NA | NA | NA | NA | NA |
| INTS7 | 611350 | NA | NA | NA | NA | 635.087 | −7.638361 | uc001hiw.2 |
| INTS8 | 611351 | NA | NA | NA | NA | 709.363 | −7.946321 | uc010mba.3 |
| INTU | 610621 | NA | NA | NA | NA | 779.084 | −8.239139 | uc003ifk.2 |
| IP6K1 | 606991 | NA | NA | NA | NA | NA | NA | NA |
| IP6K2 | 606992 | 240.226 | −9.857403 | uc003cur.3 | chr3: 48730884- | NA | NA | NA |
| | | | | | 48731581 | | | |
| IP6K3 | 606993 | 966.315 | −4.151662 | uc003ofb.2 | chr6: 33689443- | NA | NA | NA |
| | | | | | 33690496 | | | |
| IPCEF1 | NA | NA | NA | NA | NA | NA | NA | NA |
| IPMK | 609851 | NA | NA | NA | NA | 284.221 | −5.15376 | uc001jkb.3 |
| IPO13 | 610411 | 226.208 | −5.131939 | uc001ckx.3 | chr1: 44433355- | NA | NA | NA |
| | | | | | 44433694 | | | |
| IPO5 | 602008 | 633.465 | −6.185322 | uc001vne.3 | chr13: 98674077- | NA | NA | NA |
| | | | | | 98676550 | | | |
| IPO7 | 605586 | NA | NA | NA | NA | 827.878 | −14.07708 | uc001mho.3 |
| IPO9 | NA | 721.691 | −4.49076 | uc001gwz.3 | chr1: 201845183- | NA | NA | NA |
| | | | | | 201853422 | | | |
| IQCD | NA | NA | NA | NA | NA | NA | NA | NA |
| IQCG | 612477 | 908.613 | −2.613278 | uc003fyn.3 | chr3: 197615946- | 872.866 | −3.576795 | uc003fyn.3 |
| | | | | | 197616450 | | | |
| IQCH | 612523 | NA | NA | NA | NA | 506.489 | −7.872273 | uc002aqm.3 |
| IQCK | NA | NA | NA | NA | NA | 491.008 | −6.755109 | uc010vat.2 |
| IQGAP2 | 605401 | NA | NA | NA | NA | 277.834 | −6.045029 | uc003kek.3 |
| IRAK4 | 606883 | NA | NA | NA | NA | NA | NA | NA |
| IRF2 | 147576 | 1792.14 | −13.227448 | uc003iwf.4 | chr4: 185308876- | 1609.34 | −10.44082 | uc003iwf.4 |
| | | | | | 185309911 | | | |
| IRF4 | 601900 | NA | NA | NA | NA | 755.367 | −6.683249 | uc010jne.2 |
| IRF6 | 607199 | NA | NA | NA | NA | 350.771 | −10.398505 | uc001hhq.2 |
| IRF9 | 147574 | NA | NA | NA | NA | NA | NA | NA |
| IRS2 | 600797 | 715.797 | −7.669286 | uc001vqv.3 | chr13: 110406184- | NA | NA | NA |
| | | | | | 110408650 | | | |
| IRX1 | 606197 | 517.408 | −5.176329 | uc003jde.3 | chr5: 3601155- | NA | NA | NA |
| | | | | | 3601517 | | | |
| IRX2 | 606198 | NA | NA | NA | NA | NA | NA | NA |
| IRX3 | 612985 | NA | NA | NA | NA | 760.89 | −14.332879 | uc002eht.1 |
| ISL1 | 600366 | 910.091 | −6.759197 | uc003jor.3 | chr5: 50689445- | NA | NA | NA |
| | | | | | 50690563 | | | |
| ISM1 | 615793 | 1091.52 | −12.324546 | uc010gce.1 | chr20: 13280107- | NA | NA | NA |
| | | | | | 13281297 | | | |
| ISOC1 | NA | NA | NA | NA | NA | 240.605 | −5.360344 | uc003kva.3 |
| ITFG1 | 611803 | NA | NA | NA | NA | NA | NA | NA |
| ITFG3 | NA | NA | NA | NA | NA | NA | NA | NA |
| ITGA2 | 192974 | NA | NA | NA | NA | NA | NA | NA |
| ITGA8 | 604063 | NA | NA | NA | NA | 445.559 | −4.487595 | uc001ioc.1 |
| ITGA9 | 603963 | NA | NA | NA | NA | 1245.49 | −8.753277 | uc003chd.3 |
| ITGAE | 604682 | NA | NA | NA | NA | NA | NA | NA |
| ITGB1 | 135630 | 203.183 | −6.174036 | uc001iwr.4 | chr10: 33189246- | 725.606 | −8.871923 | uc001iwr.4 |
| | | | | | 33190563 | | | |
| ITGB3BP | 605494 | 743.549 | −4.880447 | uc001dba.2 | chr1: 63906441- | 568.143 | −7.676051 | uc009wak.1 |
| | | | | | 63906774 | | | |
| ITIH2 | 146640 | 279.866 | −12.122012 | uc001ijs.3 | chr10: 7791298- | NA | NA | NA |
| | | | | | 7791483 | | | |
| ITIH5 | 609783 | NA | NA | NA | NA | 1198.44 | −10.865683 | uc001ijr.2 |
| ITM2A | 300222 | 230.733 | −6.07173 | uc004edh.3 | chrX: 78615881- | NA | NA | NA |
| | | | | | 78616585 | | | |
| ITM2C | 609554 | 483.479 | −4.53937 | uc002vqz.3 | chr2: 231742815- | NA | NA | NA |
| | | | | | 231743963 | | | |
| ITPK1 | 601838 | NA | NA | NA | NA | 596.118 | −6.888427 | uc001ybf.3 |
| ITPKB | 147522 | NA | NA | NA | NA | NA | NA | NA |
| ITPR1 | 147265 | 649.177 | −10.117708 | uc003bqc.3 | chr3: 4887910- | NA | NA | NA |
| | | | | | 4889524 | | | |
| IVD | 607036 | NA | NA | NA | NA | 580.804 | −6.226191 | uc001zlq.2 |
| JADE1 | 610514 | NA | NA | NA | NA | 265.351 | −4.28772 | uc003igk.3 |
| JAK2 | 147796 | NA | NA | NA | NA | 1588.76 | −19.010233 | uc010mhm.3 |
| JAKMIP1 | 611195 | 1291.74 | −5.308066 | uc010idd.1 | chr4: 6027926- | 1071.84 | −4.560272 | uc003giu.4 |
| | | | | | 6028005 | | | |
| JMY | 604279 | NA | NA | NA | NA | NA | NA | NA |
| JPH4 | NA | 950.64 | −6.685653 | uc010tnr.1 | chr14: 24037244- | 601.675 | −6.912567 | uc001wks.2 |
| | | | | | 24038842 | | | |
| JRK | 603210 | NA | NA | NA | NA | NA | NA | NA |
| JRKL | 603211 | 978.023 | −19.332694 | uc001pfy.2 | chr11: 96125389- | 761.377 | −8.674533 | uc001pfy.2 |
| | | | | | 96126727 | | | |
| KANSL1L | 613833 | 933.627 | −9.278384 | uc002vdx.1 | chr2: 210908157- | 755.362 | −12.023713 | uc002vdy.1 |
| | | | | | 210908636 | | | |
| KANSL2 | 615488 | NA | NA | NA | NA | 1375.59 | −9.780064 | uc001rrw.2 |
| KANSL3 | NA | NA | NA | NA | NA | 974.194 | −7.726172 | uc002swo.3 |
| KARS | 601421 | 397.406 | −2.820397 | uc002feq.3 | chr16: 75661622- | NA | NA | NA |
| | | | | | 75661792 | | | |
| KAT2B | 602303 | NA | NA | NA | NA | NA | NA | NA |
| KAT7 | 609880 | NA | NA | NA | NA | 621.26 | −6.428508 | uc002ipl.2 |
| KATNBL1 | 616235 | NA | NA | NA | NA | NA | NA | NA |
| KAZN | NA | NA | NA | NA | NA | NA | NA | NA |
| KBTBD2 | NA | 353.007 | −4.989817 | uc003tdb.2 | chr7: 32907778- | NA | NA | NA |
| | | | | | 32908956 | | | |
| KBTBD4 | NA | NA | NA | NA | NA | 335.238 | −4.216616 | uc001nfw.2 |
| KCNC1 | 176258 | NA | NA | NA | NA | NA | NA | NA |
| KCND1 | 300281 | 532.142 | −7.98407 | uc004dlw.1 | chrX: 48818639- | NA | NA | NA |
| | | | | | 48819841 | | | |
| KCNG3 | 606767 | 993.685 | −8.503403 | uc002rsm.3 | chr2: 42669157- | NA | NA | NA |
| | | | | | 42671073 | | | |
| KCNH5 | 605716 | 263.383 | −7.70718 | uc001xga.3 | chr14: 63416531- | 1906.06 | −20.492517 | uc001xfx.3 |
| | | | | | 63416776 | | | |
| KCNH7 | 608169 | NA | NA | NA | NA | NA | NA | NA |
| KCNJ13 | 603208 | NA | NA | NA | NA | 1391.06 | −10.320448 | uc002vto.3 |
| KCNK10 | 605873 | NA | NA | NA | NA | 213.577 | −10.016696 | uc001xwm.3 |
| KCNK12 | 607366 | NA | NA | NA | NA | 1386.97 | −7.833361 | uc002rwb.3 |
| KCNK17 | 607370 | 208.971 | −5.130159 | uc003ooo.3 | chr6: 39266777- | NA | NA | NA |
| | | | | | 39267202 | | | |
| KGNK6 | 603939 | NA | NA | NA | NA | 463.412 | −4.133243 | uc002oic.3 |
| KCNK7 | 603940 | NA | NA | NA | NA | NA | NA | NA |
| KCNMB2 | 605214 | NA | NA | NA | NA | NA | NA | NA |
| KCNRG | 607947 | 934.526 | −8.63731 | uc001vdt.3 | chr13: 50594364- | 433.327 | −4.626173 | uc001vdu.3 |
| | | | | | 50595058 | | | |
| KCNS1 | 602905 | 447.41 | −6.230584 | uc002xnc.3 | chr20: 43720950- | NA | NA | NA |
| | | | | | 43723510 | | | |
| KCNS3 | 603888 | NA | NA | NA | NA | 483.574 | −4.280391 | uc002rcv.3 |
| KCNT2 | 610044 | 959.972 | −11.5574 | uc009wyu.3 | chr1: 196249674- | NA | NA | NA |
| | | | | | 196249977 | | | |
| KCNU1 | 615215 | NA | NA | NA | NA | 914.904 | −9.653798 | uc010lvw.3 |
| KCTD1 | 613420 | 393.021 | −5.912629 | uc002kvy.3 | chr18: 24035684- | NA | NA | NA |
| | | | | | 24039577 | | | |
| KCTD18 | NA | NA | NA | NA | NA | 313.366 | −7.282142 | uc002uvs.3 |
| KCTD20 | 615932 | 652.783 | −4.943606 | uc003ome.3 | chr6: 36454953- | NA | NA | NA |
| | | | | | 36458315 | | | |
| KCTD5 | 611285 | NA | NA | NA | NA | NA | NA | NA |
| KCTD6 | NA | NA | NA | NA | NA | 1414.48 | −8.229314 | uc003dkj.4 |
| KCTD7 | 611725 | 268.402 | −5.637918 | uc003tve.3 | chr7: 66104220- | 360.444 | −6.540856 | uc003tvd.4 |
| | | | | | 66108216 | | | |
| KDELR2 | 609024 | 579.629 | −6.780667 | uc003sqe.4 | chr7: 6500712- | NA | NA | NA |
| | | | | | 6502771 | | | |
| KDM1A | 609132 | NA | NA | NA | NA | NA | NA | NA |
| KDM4E | 616581 | NA | NA | NA | NA | 357.376 | −4.13108 | uc010ruf.1 |
| KDM5A | 180202 | NA | NA | NA | NA | NA | NA | NA |
| KDM5D | 426000 | NA | NA | NA | NA | 964.02 | −2.583996 | uc004fuf.3 |
| KEAP1 | 606016 | NA | NA | NA | NA | 536.565 | −8.842705 | uc002mop.1 |
| KEL | 613883 | NA | NA | NA | NA | 567.874 | −5.355261 | uc003wcb.3 |
| KHDC1 | 611688 | 489.964 | −5.080418 | uc003pgn.4 | chr6: 73951038- | NA | NA | NA |
| | | | | | 73951251 | | | |
| KHDC1L | NA | NA | NA | NA | NA | 494.219 | −5.112054 | uc003pgm.4 |
| KHDRBS2 | 610487 | 681.676 | −7.145035 | uc003peg.2 | chr6: 62389865- | NA | NA | NA |
| | | | | | 62390867 | | | |
| KIAA0100 | 610664 | 1353.13 | −2.175733 | uc002hbu.3 | chr17: 26941458- | NA | NA | NA |
| | | | | | 26942081 | | | |
| KIAA0101 | 610696 | NA | NA | NA | NA | NA | NA | NA |
| KIAA0196 | 610657 | 548.898 | −4.805341 | uc003yrt.3 | chr8: 126036503- | NA | NA | NA |
| | | | | | 126036858 | | | |
| KIAA0226 | NA | 650.318 | −6.160363 | uc003fyc.2 | chr3: 197398259- | 883.416 | −10.352966 | uc003fyc.2 |
| | | | | | 197401888 | | | |
| KIAA0368 | 616694 | 497.34 | −7.791023 | uc004bfe.1 | chr9: 114122973- | NA | NA | NA |
| | | | | | 114124309 | | | |
| KIAA0408 | NA | NA | NA | NA | NA | 581.525 | −5.358621 | uc003qba.3 |
| KIAA0513 | 611675 | NA | NA | NA | NA | NA | NA | NA |
| KIAA0586 | 610178 | 575.156 | −11.705671 | uc001xdt.4 | chr14: 59014651- | 568.392 | −6.827958 | uc001xdt.4 |
| | | | | | 59015549 | | | |
| KIAA0754 | NA | NA | NA | NA | NA | NA | NA | NA |
| KIAA0825 | NA | NA | NA | NA | NA | NA | NA | NA |
| KIAA0895 | NA | NA | NA | NA | NA | 592.762 | −8.383723 | uc003tfe.3 |
| KIAA0922 | 616243 | 1156.62 | −6.964402 | NA | NA | NA | NA | NA |
| KIAA1024 | NA | NA | NA | NA | NA | 721.416 | −3.744789 | uc002bew.1 |
| KIAA1107 | NA | NA | NA | NA | NA | NA | NA | NA |
| KIAA1143 | NA | NA | NA | NA | NA | NA | NA | NA |
| KIAA1147 | NA | NA | NA | NA | NA | NA | NA | NA |
| KIAA1191 | NA | NA | NA | NA | NA | 399.619 | −8.77982 | uc003mdw.3 |
| KIAA1210 | NA | 960.002 | −9.57114 | uc004era.4 | chrX: 118212598- | 1268.04 | −10.066452 | uc004era.4 |
| | | | | | 118215291 | | | |
| KIAA1211 | NA | 783.994 | −7.690833 | uc003hbk.2 | chr4: 57193971- | NA | NA | NA |
| | | | | | 57196890 | | | |
| KIAA1217 | NA | 1137.35 | −6.858569 | uc001irs.3 | chr10: 24835254- | NA | NA | NA |
| | | | | | 24836772 | | | |
| KIAA1257 | NA | NA | NA | NA | NA | 272.378 | −5.503552 | uc003eli.4 |
| KIAA1324 | 611298 | NA | NA | NA | NA | NA | NA | NA |
| KIAA1328 | 616480 | NA | NA | NA | NA | 471.208 | −0.996908 | uc021uiw.1 |
| KIAA1407 | NA | NA | NA | NA | NA | NA | NA | NA |
| KIAA1429 | 616447 | NA | NA | NA | NA | 206.337 | −5.490174 | uc003ygo.2 |
| KIAA1462 | 614398 | NA | NA | NA | NA | 1047.81 | −7.776178 | uc001iux.3 |
| KIAA1468 | NA | NA | NA | NA | NA | NA | NA | NA |
| KIAA1551 | NA | NA | NA | NA | NA | 429.619 | −5.539283 | uc001rks.3 |
| KIAA1586 | NA | NA | NA | NA | NA | 675.408 | −9.069655 | uc003pdj.3 |
| KIAA1598 | NA | NA | NA | NA | NA | 507.129 | −5.36657 | uc021pzk.1 |
| KIAA1715 | 610236 | NA | NA | NA | NA | NA | NA | NA |
| KIAA1755 | NA | 704.057 | −4.433232 | uc002xhv.1 | chr20: 36838907- | NA | NA | NA |
| | | | | | 36841443 | | | |
| KIAA1841 | NA | NA | NA | NA | NA | 612.256 | −6.451747 | uc002sav.4 |
| KIAA1875 | NA | 832.178 | −4.965117 | uc011lkz.1 | chr8: 145171197- | NA | NA | NA |
| | | | | | 145173218 | | | |
| KIAA2013 | NA | 596.191 | −5.986481 | uc001atl.2 | chr1: 11982232- | NA | NA | NA |
| | | | | | 11982575 | | | |
| KIAA2018 | NA | NA | NA | NA | NA | NA | NA | NA |
| KIAA2022 | 300524 | 578.014 | −10.608828 | uc004eby.3 | chrX: 73952691- | 752.901 | −8.576447 | uc004eby.3 |
| | | | | | 73959239 | | | |
| KIF11 | 148760 | 514.585 | −5.167591 | uc001kic.3 | chr10: 94413554- | NA | NA | NA |
| | | | | | 94415152 | | | |
| KIF15 | NA | NA | NA | NA | NA | NA | NA | NA |
| KIF16B | NA | NA | NA | NA | NA | NA | NA | NA |
| KIF17 | 605037 | 751.297 | −5.284611 | uc001bdp.4 | chr1: 20990507- | NA | NA | NA |
| | | | | | 20991076 | | | |
| KIF20B | 605498 | NA | NA | NA | NA | 1502.92 | −17.615404 | uc001kgr.1 |
| KIF23 | 605064 | 882.061 | −6.249868 | uc002asb.3 | chr15: 69740147- | NA | NA | NA |
| | | | | | 69740764 | | | |
| KIF25 | 603815 | NA | NA | NA | NA | NA | NA | NA |
| KIF26B | 614026 | 387.886 | −7.606735 | uc001ibf.1 | chr1: 245865909- | NA | NA | NA |
| | | | | | 245866428 | | | |
| KIF2B | 615142 | NA | NA | NA | NA | 252.96 | −5.341407 | uc002iua.2 |
| KIF5A | 602821 | 538.279 | −6.302238 | uc001sor.1 | chr12: 57978001- | NA | NA | NA |
| | | | | | 57978554 | | | |
| KIFC1 | 603763 | 1576.87 | −26.78724 | uc003oef.4 | chr6: 33377468- | NA | NA | NA |
| | | | | | 33377699 | | | |
| KIN | 601720 | NA | NA | NA | NA | NA | NA | NA |
| KIR2DL5B | 615727 | NA | NA | NA | NA | NA | NA | NA |
| KIR3DX1 | NA | NA | NA | NA | NA | NA | NA | NA |
| KISS1R | 604161 | NA | NA | NA | NA | NA | NA | NA |
| KITLG | 184745 | 496.458 | −7.844421 | uc009zsn.3 | chr12: 88886570- | 428.93 | −10.028932 | uc009zsn.3 |
| | | | | | 88890888 | | | |
| KL | 604824 | NA | NA | NA | NA | NA | NA | NA |
| KLF3 | 609392 | 709.181 | −10.823703 | uc003gtg.2 | chr4: 38691505- | NA | NA | NA |
| | | | | | 38692372 | | | |
| KLF4 | 602253 | 682.472 | −5.102033 | uc004bdf.2 | chr9: 110247133- | 582.265 | −4.486817 | uc004bdf.2 |
| | | | | | 110248031 | | | |
| KLF8 | 300286 | 261.43 | −3.171367 | uc010nkg.2 | chrX: 56292186- | 1476.46 | −8.70384 | uc010nkg.2 |
| | | | | | 56295922 | | | |
| KLHDC1 | 611281 | 1340.26 | −17.533052 | uc001www.3 | chr14: 50218491- | NA | NA | NA |
| | | | | | 50219870 | | | |
| KLHDC10 | 615152 | NA | NA | NA | NA | NA | NA | NA |
| KLHDG8B | 613169 | NA | NA | NA | NA | NA | NA | NA |
| KLHL1 | 605332 | 1563.77 | −13.303817 | uc001vip.3 | chr13: 70274725- | 1089.43 | −6.50114 | uc001vip.3 |
| | | | | | 70275833 | | | |
| KLHL10 | 608778 | 566.702 | −5.234499 | uc010wfv.1 | chr17: 40002053- | NA | NA | NA |
| | | | | | 40002185 | | | |
| KLHL12 | 614522 | NA | NA | NA | NA | NA | NA | NA |
| KLHL2 | 605774 | NA | NA | NA | NA | NA | NA | NA |
| KLHL20 | NA | 713.338 | −5.267639 | uc001gjc.3 | chr1: 173754386- | NA | NA | NA |
| | | | | | 173755840 | | | |
| KLHL24 | 611295 | NA | NA | NA | NA | NA | NA | NA |
| KLHL25 | NA | NA | NA | NA | NA | NA | NA | NA |
| KLHL3 | 605775 | NA | NA | NA | NA | 704.325 | −6.186347 | uc003lbs.1 |
| KLHL32 | NA | NA | NA | NA | NA | 233.472 | −4.313838 | uc003poy.3 |
| KLHL36 | NA | NA | NA | NA | NA | NA | NA | NA |
| KLHL41 | 607701 | NA | NA | NA | NA | NA | NA | NA |
| KLHL6 | 614214 | NA | NA | NA | NA | NA | NA | NA |
| KLK10 | 602673 | 512.228 | −6.425521 | uc002puy.3 | chr19: 51516000- | NA | NA | NA |
| | | | | | 51518055 | | | |
| KLK11 | 604434 | NA | NA | NA | NA | 1040.06 | −9.007307 | uc002pvc.4 |
| KLK12 | 605539 | NA | NA | NA | NA | 845.803 | −8.657449 | uc010ycs.1 |
| KLRC2 | 602891 | NA | NA | NA | NA | 836.026 | −8.403813 | uc010she.1 |
| KLRC4 | 602893 | NA | NA | NA | NA | NA | NA | NA |
| KLRD1 | 602894 | 1071.03 | −3.515876 | uc001qxw.4 | chr12: 10467393- | NA | NA | NA |
| | | | | | 10469850 | | | |
| KLRG2 | NA | NA | NA | NA | NA | 705.659 | −6.44214 | uc003vvb.3 |
| KMT2C | 606833 | 350.129 | −4.630178 | uc003wkx.3 | chr7: 151832010- | NA | NA | NA |
| | | | | | 151833916 | | | |
| KMT2E | 608444 | 480.701 | −0.378577 | uc003vcl.3 | chr7: 104731870- | 552.731 | −6.357429 | uc003vcl.3 |
| | | | | | 104732057 | | | |
| KNG1 | 612358 | 919.417 | −10.901619 | uc011bsa.2 | chr3: 186460121- | NA | NA | NA |
| | | | | | 186460678 | | | |
| KPRP | 613260 | NA | NA | NA | NA | 534.379 | −6.272196 | uc001fal.1 |
| KRAS | 190070 | 372.819 | −4.225845 | uc001rgp.1 | chr12: 25358180- | NA | NA | NA |
| | | | | | 25362845 | | | |
| KRBA2 | NA | NA | NA | NA | NA | 364.448 | −9.110276 | uc002glf.1 |
| KREMEN1 | 609898 | NA | NA | NA | NA | NA | NA | NA |
| KRIT1 | 604214 | NA | NA | NA | NA | 503.875 | −4.549532 | uc010lev.1 |
| KRT1 | 139350 | NA | NA | NA | NA | 206.86 | −5.112054 | uc001sau.1 |
| KRT12 | 601687 | NA | NA | NA | NA | 1059.76 | −7.476569 | uc002hvk.2 |
| KRT23 | 606194 | NA | NA | NA | NA | 940.325 | −10.901081 | uc010cxg.3 |
| KRT28 | 616677 | NA | NA | NA | NA | NA | NA | NA |
| KRT73 | 608247 | 242.646 | −5.091727 | uc001sas.3 | chr12: 53001354- | NA | NA | NA |
| | | | | | 53001979 | | | |
| KRTCAP2 | NA | 865.644 | −5.952069 | uc001fhp.1 | chr1: 155143877- | NA | NA | NA |
| | | | | | 155145032 | | | |
| KSR1 | 601132 | NA | NA | NA | NA | NA | NA | NA |
| KYNU | 605197 | 1309.44 | −11.326714 | uc002tvk.3 | chr2: 143746911- | NA | NA | NA |
| | | | | | 143747106 | | | |
| L3HYPDH | 614811 | 413.438 | −4.933639 | uc010trx.1 | chr14: 59945444- | 618.697 | −11.405501 | uc001xee.1 |
| | | | | | 59946069 | | | |
| LACC1 | 613409 | NA | NA | NA | NA | 1371.37 | −10.96318 | uc010acg.3 |
| LACE1 | NA | NA | NA | NA | NA | 1099.9 | −16.617198 | uc003psj.3 |
| LACTB2 | NA | 607.268 | −7.957961 | uc003xyp.3 | chr8: 71549501- | 1738.32 | −8.999381 | uc003xyp.3 |
| | | | | | 71550094 | | | |
| LAG3 | 153337 | 388.552 | −4.825936 | uc001qqs.3 | chr12: 6884741- | NA | NA | NA |
| | | | | | 6884897 | | | |
| LALBA | 149750 | 428.413 | −5.499084 | uc001rrt.3 | chr12: 48961467- | NA | NA | NA |
| | | | | | 48961739 | | | |
| LAMA2 | 156225 | NA | NA | NA | NA | 906.517 | −9.149238 | uc003qbn.3 |
| LAMA5 | 601033 | NA | NA | NA | NA | NA | NA | NA |
| LAMB4 | 616380 | 758.739 | −4.568145 | uc003vey.2 | chr7: 107663996- | NA | NA | NA |
| | | | | | 107664483 | | | |
| LAMP2 | 309060 | 460.246 | −7.746153 | uc004ess.4 | chrX: 119560003- | 771.212 | −6.625496 | uc004ess.4 |
| | | | | | 119562338 | | | |
| LAMTOR3 | 603296 | 890.596 | −10.386552 | uc003hvg.2 | chr4: 100799495- | 212.15 | −7.092043 | uc003hvg.2 |
| | | | | | 100803150 | | | |
| LAPTM4A | NA | 845.172 | −5.944465 | uc002rdm.3 | chr2: 20232411- | NA | NA | NA |
| | | | | | 20232965 | | | |
| LARP1B | NA | 689.253 | −1.28018 | uc003ify.3 | chr4: 129029058- | NA | NA | NA |
| | | | | | 129030056 | | | |
| LARP6 | 611300 | NA | NA | NA | NA | 512.237 | −5.755202 | uc002ass.3 |
| LARS | 151350 | NA | NA | NA | NA | 296.768 | −6.345379 | uc003lnw.1 |
| LASP1 | 602920 | NA | NA | NA | NA | 483.935 | −6.096677 | uc010wdy.1 |
| LATS1 | 603473 | 963.649 | −6.920701 | uc003qmw.3 | chr6: 150004055- | 758.185 | −8.290386 | uc003qmw.3 |
| | | | | | 150004151 | | | |
| LBR | 600024 | NA | NA | NA | NA | 1651.9 | −8.394796 | uc001hoy.3 |
| LCA5 | 611408 | 234.832 | −5.294173 | uc003pix.3 | chr6: 80194708- | 643.097 | −5.361305 | uc011dyr.2 |
| | | | | | 80196720 | | | |
| LCE1B | 612604 | NA | NA | NA | NA | 596.869 | −5.920294 | uc001faq.3 |
| LCE1E | 612607 | NA | NA | NA | NA | 582.176 | −6.777891 | uc001fan.3 |
| LCE2A | 612609 | 320.507 | −4.45297 | uc001faj.3 | chr1: 152671699- | NA | NA | NA |
| | | | | | 152671918 | | | |
| LCE3D | 612616 | NA | NA | NA | NA | NA | NA | NA |
| LCLAT1 | 614241 | NA | NA | NA | NA | 459.766 | −4.588473 | uc002rnk.1 |
| LCN12 | 612905 | NA | NA | NA | NA | 380.616 | −4.355081 | uc004ckc.3 |
| LCOR | 607698 | NA | NA | NA | NA | 601.835 | −6.894172 | uc001kmr.3 |
| LCORL | 611799 | 1525.42 | −13.872596 | uc021xmq.1 | chr4: 17844839- | 998.571 | −20.203447 | uc021xmq.1 |
| | | | | | 17847343 | | | |
| LDB1 | 603451 | NA | NA | NA | NA | 501.662 | −4.343469 | uc001kul.3 |
| LDLRAD3 | NA | 217.07 | −6.369407 | uc001mwk.1 | chr11: 36250948- | NA | NA | NA |
| | | | | | 36252841 | | | |
| LDLRAD4 | 606571 | NA | NA | NA | NA | NA | NA | NA |
| LDOC1 | 300402 | NA | NA | NA | NA | 518.406 | −4.466028 | uc004fbj.3 |
| LEAP2 | 611373 | NA | NA | NA | NA | NA | NA | NA |
| LEFTY1 | 603037 | 378.903 | −4.477337 | uc001hpo.3 | chr1: 226073982- | NA | NA | NA |
| | | | | | 226074426 | | | |
| LEKR1 | 613536 | NA | NA | NA | NA | NA | NA | NA |
| LEMD1 | 610480 | 1128.42 | −6.387362 | uc001hci.2 | chr1: 205350506- | NA | NA | NA |
| | | | | | 205350862 | | | |
| LEMD3 | 607844 | 1206.3 | −5.9547 | uc001ssl.2 | chr12: 65640106- | NA | NA | NA |
| | | | | | 65642141 | | | |
| LEO1 | 610507 | 831.311 | −8.764199 | uc002abo.3 | chr15: 52230222- | NA | NA | NA |
| | | | | | 52230352 | | | |
| LEPROT | 613461 | NA | NA | NA | NA | NA | NA | NA |
| LEPROTL1 | 607338 | NA | NA | NA | NA | 898.329 | −12.283378 | uc003xhw.4 |
| LETM1 | 604407 | NA | NA | NA | NA | NA | NA | NA |
| LETMD1 | NA | NA | NA | NA | NA | 948.617 | −5.505503 | uc010sna.2 |
| LGALS13 | 608717 | NA | NA | NA | NA | NA | NA | NA |
| LGALS3 | 153619 | 407.462 | −4.042018 | uc001xbr.3 | chr14: 55611990- | NA | NA | NA |
| | | | | | 55612148 | | | |
| LGALS8 | 606099 | 927.675 | −10.702852 | uc001hxw.2 | chr1: 236711462- | 767.442 | −7.210276 | uc001hxw.2 |
| | | | | | 236716279 | | | |
| LGI4 | 608303 | NA | NA | NA | NA | 844.553 | −6.715664 | uc002nxx.2 |
| LHFP | 606710 | 529.647 | −8.710191 | uc001uxf.3 | chr13: 39917029- | NA | NA | NA |
| | | | | | 39918072 | | | |
| LHX8 | 604425 | NA | NA | NA | NA | NA | NA | NA |
| LHX9 | 606066 | NA | NA | NA | NA | 1592.21 | −13.08933 | uc001gui.1 |
| LIG1 | 126391 | NA | NA | NA | NA | NA | NA | NA |
| LIG3 | 600940 | NA | NA | NA | NA | 739.618 | −6.519317 | uc002hij.3 |
| LILRA1 | 604810 | 285.122 | −7.432334 | uc010yfh.2 | chr19: 55110760- | 441.019 | −3.455055 | uc010yfh.2 |
| | | | | | 55111185 | | | |
| LILRA2 | 604812 | NA | NA | NA | NA | 1114.18 | −2.452249 | uc010ern.3 |
| LILRB2 | 604815 | 320.219 | −4.215396 | uc010eri.3 | chr19: 54777675- | NA | NA | NA |
| | | | | | 54778683 | | | |
| LIMA1 | 608364 | 436.621 | −5.254071 | uc001rwg.4 | chr12: 50569563- | NA | NA | NA |
| | | | | | 50570846 | | | |
| LIMCH1 | NA | 741.562 | −5.98331 | uc003gvt.1 | chr4: 41621462- | NA | NA | NA |
| | | | | | 41622311 | | | |
| LIMK1 | 601329 | NA | NA | NA | NA | NA | NA | NA |
| LIMS1 | 602567 | NA | NA | NA | NA | 1080 | −6.761596 | uc002tel.3 |
| LIN28A | 611043 | NA | NA | NA | NA | 636.894 | −5.312572 | uc001bmj.3 |
| LIN7B | 612331 | NA | NA | NA | NA | 889.025 | −7.133192 | NA |
| LIN7C | 612332 | 763.403 | −6.825205 | uc001mrl.3 | chr11: 27515965- | NA | NA | NA |
| | | | | | 27520195 | | | |
| LINC00167 | NA | NA | NA | NA | NA | NA | NA | NA |
| LINC00305 | NA | NA | NA | NA | NA | 1142.51 | −11.395425 | uc010dqk.2 |
| LING00493 | NA | NA | NA | NA | NA | 429.445 | −8.941192 | uc002wrd.4 |
| LINC00657 | NA | NA | NA | NA | NA | NA | NA | NA |
| LINC00893 | NA | 1057.76 | −5.003196 | uc011mxl.1 | chrX: 148614964- | NA | NA | NA |
| | | | | | 148615450 | | | |
| LINC00937 | NA | NA | NA | NA | NA | 792.425 | −4.711658 | uc001quk.4 |
| LINC00998 | NA | NA | NA | NA | NA | 1196.01 | −10.697528 | uc011kmt.2 |
| LINC01225 | NA | 228.97 | −4.523737 | uc021okn.1 | chr1: 31973448- | NA | NA | NA |
| | | | | | 31974167 | | | |
| LINC01387 | NA | 513.96 | −6.048592 | uc021ugs.1 | chr18: 6590544- | NA | NA | NA |
| | | | | | 6590652 | | | |
| LINC01559 | NA | NA | NA | NA | NA | NA | NA | NA |
| LINC01588 | NA | NA | NA | NA | NA | 709.54 | −10.366445 | uc001wxi.1 |
| LIPF | 601980 | NA | NA | NA | NA | 1154.58 | −11.42977 | uc009xtk.3 |
| LIX1L | NA | 465.506 | −4.127138 | uc001enr.3 | chr1: 145498779- | NA | NA | NA |
| | | | | | 145499091 | | | |
| LMAN1 | 601567 | 426.858 | −7.056036 | uc002lhz.3 | chr18: 56995056- | 697.89 | −4.746183 | uc002lhz.3 |
| | | | | | 56998324 | | | |
| LMAN2 | 609551 | NA | NA | NA | NA | 541.13 | −5.083871 | uc003mge.3 |
| LMAN2 | 609552 | NA | NA | NA | NA | NA | NA | NA |
| LMBR1 | 605522 | 595.619 | −4.557091 | uc003wmv.4 | chr7: 156473570- | NA | NA | NA |
| | | | | | 156476771 | | | |
| LMBR1L | 610007 | NA | NA | NA | NA | NA | NA | NA |
| LMBRD1 | 612625 | NA | NA | NA | NA | NA | NA | NA |
| LMLN | 609380 | NA | NA | NA | NA | 1496.96 | −8.755524 | uc003fyt.3 |
| LMNB1 | 150340 | NA | NA | NA | NA | NA | NA | NA |
| LMNTD1 | NA | 477.755 | −5.233399 | uc009zjc.2 | chr12: 25652786- | NA | NA | NA |
| | | | | | 25652930 | | | |
| LMO3 | 180386 | 597.343 | −6.437445 | uc001rdj.2 | chr12: 16701306- | NA | NA | NA |
| | | | | | 16704155 | | | |
| LMO7 | 604362 | 395.721 | −5.964015 | uc010thv.2 | chr13: 76432080- | 1229.09 | −9.859924 | uc010thv.2 |
| | | | | | 76434006 | | | |
| LNP1 | NA | NA | NA | NA | NA | 377.745 | −4.07697 | uc003dtx.4 |
| LNX2 | 609733 | NA | NA | NA | NA | 671.306 | −5.469355 | uc001url.4 |
| LONRF2 | NA | 680.04 | −4.919167 | uc002tal.4 | chr2: 100889753- | 277.639 | −8.871164 | uc002tal.4 |
| | | | | | 100900759 | | | |
| LPAR2 | 605110 | NA | NA | NA | NA | 322.546 | −5.175553 | uc002nna.4 |
| LPAR4 | 300086 | NA | NA | NA | NA | 706.262 | −6.487599 | uc010nme.3 |
| LPCAT4 | 612039 | 537.804 | −5.137233 | uc001zig.3 | chr15: 34651089- | 750.42 | −10.487956 | uc001zig.3 |
| | | | | | 34651327 | | | |
| LPP | 600700 | 595.339 | −7.424327 | uc011bsg.2 | chr3: 188592268- | NA | NA | NA |
| | | | | | 188608460 | | | |
| LPPR1 | NA | NA | NA | NA | NA | NA | NA | NA |
| LPPR5 | NA | 825.445 | −6.123556 | uc001dsb.3 | chr1: 99355801- | NA | NA | NA |
| | | | | | 99358627 | | | |
| LRAT | 604863 | NA | NA | NA | NA | 658.477 | −15.322758 | uc003iom.1 |
| LRBA | 606453 | 486.923 | −6.830625 | uc003ilx.2 | chr4: 151933981- | 282.531 | −6.731226 | uc003ilr.4 |
| | | | | | 151935566 | | | |
| LRCOL1 | NA | 609.117 | −6.052808 | uc021rgr.1 | chr12: 133179736- | NA | NA | NA |
| | | | | | 133179987 | | | |
| LRMP | 602003 | NA | NA | NA | NA | NA | NA | NA |
| LRP11 | NA | NA | NA | NA | NA | 546.525 | −5.386727 | uc003qng.2 |
| LRP12 | NA | NA | NA | NA | NA | 960.494 | −7.841613 | uc003ylz.3 |
| LRP1B | 608766 | 661.759 | −10.268328 | uc002tvj.1 | chr2: 140988996- | NA | NA | NA |
| | | | | | 140990754 | | | |
| LRP2 | 600073 | NA | NA | NA | NA | NA | NA | NA |
| LRP2BP | NA | NA | NA | NA | NA | 1617.05 | −13.409775 | uc003ixj.2 |
| LRP4 | 604270 | NA | NA | NA | NA | 691.399 | −8.751679 | uc001ndm.4 |
| LRPPRC | 607544 | 757.21 | −10.056367 | uc002rtr.2 | chr2: 44113363- | 1482.5 | −18.658574 | uc002rtr.2 |
| | | | | | 44115738 | | | |
| LRR1 | 609193 | NA | NA | NA | NA | 347.979 | −5.937339 | uc001wwo.3 |
| LRRC10 | 610846 | NA | NA | NA | NA | NA | NA | NA |
| LRRC2 | 607180 | NA | NA | NA | NA | 401.804 | −6.20854 | uc010hji.3 |
| LRRC23 | NA | NA | NA | NA | NA | NA | NA | NA |
| LRRC27 | NA | 824.384 | −3.11674 | uc001llf.2 | chr10: 134166410- | 498.203 | −4.048622 | uc001llf.2 |
| | | | | | 134166901 | | | |
| LRRC3 | NA | NA | NA | NA | NA | 743.282 | −7.985589 | uc002zfa.3 |
| LRRC34 | NA | 980.137 | −10.347876 | uc003ffx.3 | chr3: 169511216- | NA | NA | NA |
| | | | | | 169511422 | | | |
| LRRC36 | NA | NA | NA | NA | NA | 447.368 | −5.561929 | uc002esv.3 |
| LRRC37A4P | NA | NA | NA | NA | NA | NA | NA | NA |
| LRRC4 | 610486 | 1209.6 | −8.495508 | uc003vmk.3 | chr7: 127667124- | NA | NA | NA |
| | | | | | 127668731 | | | |
| LRRC40 | NA | NA | NA | NA | NA | 998.669 | −7.962972 | uc001der.2 |
| LRRC46 | NA | 237.744 | −5.813961 | uc002ima.3 | chr17: 45914487- | NA | NA | NA |
| | | | | | 45915079 | | | |
| LRRC47 | NA | NA | NA | NA | NA | 685.736 | −5.115382 | uc001akx.1 |
| LRRC59 | 614854 | NA | NA | NA | NA | 728.562 | −6.944018 | uc002iqt.3 |
| LRRC61 | NA | 292.554 | −4.273567 | uc003wgw.3 | chr7: 150034731- | NA | NA | NA |
| | | | | | 150035245 | | | |
| LRRC66 | NA | 536.16 | −7.452762 | uc003gzi.3 | chr4: 52859866- | NA | NA | NA |
| | | | | | 52860544 | | | |
| LRRC69 | NA | NA | NA | NA | NA | NA | NA | NA |
| LRRFIP2 | 614043 | 1425.46 | −10−538168 | uc011ayf.1 | chr3: 37094117- | NA | NA | NA |
| | | | | | 37095341 | | | |
| LRRIQ1 | NA | 564.221 | −8.728813 | uc001tad.3 | chr12: 85441300- | NA | NA | NA |
| | | | | | 85442338 | | | |
| LRRK2 | 609007 | 1040.72 | −2.105601 | uc001rmg.4 | chr12: 40761568- | 623.714 | −7.817479 | uc001rmi.3 |
| | | | | | 40763086 | | | |
| LRRN1 | NA | NA | NA | NA | NA | 1486.11 | −13.838965 | uc003bpt.4 |
| LRRTM1 | 610867 | NA | NA | NA | NA | NA | NA | NA |
| LRRTM2 | 610868 | NA | NA | NA | NA | NA | NA | NA |
| LRTM1 | NA | 523.272 | −5.371038 | uc003dhl.3 | chr3: 54952381- | NA | NA | NA |
| | | | | | 54952485 | | | |
| LSM6 | 607286 | 639.088 | −7.161855 | uc003ikq.4 | chr4: 147110809- | 898.732 | −8.420926 | uc003ikq.4 |
| | | | | | 147111213 | | | |
| LSM7 | 607287 | 220.475 | −6.51192 | uc002lvp.4 | chr19: 2321520- | NA | NA | NA |
| | | | | | 2321678 | | | |
| LSMEM2 | NA | 1292.26 | −8.590169 | uc003cyz.3 | chr3: 50324634- | NA | NA | NA |
| | | | | | 50325545 | | | |
| LSR | 616582 | 1087.01 | −13.121391 | uc002nyl.3 | chr19: 35758763- | NA | NA | NA |
| | | | | | 35758867 | | | |
| LST1 | 109170 | 556.057 | −8.242802 | uc010jsw.3 | chr6: 31556518- | NA | NA | NA |
| | | | | | 31556686 | | | |
| LTV1 | NA | 539.288 | −4.08791 | uc003qjs.3 | chr6: 144184665- | NA | NA | NA |
| | | | | | 144184943 | | | |
| LUC7L2 | 613056 | NA | NA | NA | NA | NA | NA | NA |
| LUM | 600616 | 1464.68 | −17.763327 | uc001tbm.3 | chr12: 91497232- | 1752.72 | −19.863609 | uc001tbm.3 |
| | | | | | 91497941 | | | |
| LURAP1 | 616129 | 691.332 | −8.603814 | uc010oma.2 | chr1: 46685893- | NA | NA | NA |
| | | | | | 46686928 | | | |
| LURAP1L | 616130 | NA | NA | NA | NA | NA | NA | NA |
| LUZP1 | 601422 | 462.591 | −6.682476 | uc001bgk.2 | chr1: 23410516- | NA | NA | NA |
| | | | | | 23415387 | | | |
| LUZP4 | 300616 | 1613.07 | −9.499443 | uc004eqa.3 | chrX: 114541370- | NA | NA | NA |
| | | | | | 114542121 | | | |
| LVRN | 610046 | NA | NA | NA | NA | NA | NA | NA |
| LY6K | 615093 | NA | NA | NA | NA | 1200.32 | −7.719585 | uc011ljv.2 |
| LY75 | 604524 | NA | NA | NA | NA | 230.269 | −5.886763 | uc002ubc.4 |
| LYAR | NA | 1026.03 | −5.931364 | uc011bvx.2 | chr4: 4269429- | NA | NA | NA |
| | | | | | 4269615 | | | |
| LYPD6 | 613359 | NA | NA | NA | NA | NA | NA | NA |
| LYRM4 | 613311 | NA | NA | NA | NA | 495.646 | −7.06625 | uc003mwp.3 |
| LYRM7 | 615831 | NA | NA | NA | NA | 531.339 | −8.478199 | uc003kvg.1 |
| LYSMD3 | NA | 261.588 | −5.094939 | uc003kjr.3 | chr5: 89811445- | NA | NA | NA |
| | | | | | 89814635 | | | |
| LYZ | 153450 | NA | NA | NA | NA | 962.949 | −6.518174 | uc001suw.2 |
| LYZL6 | 612751 | NA | NA | NA | NA | NA | NA | NA |
| LZIC | 610458 | 1181.62 | −11.077533 | uc001aqm.3 | chr1: 9989776- | 776.499 | −7.476231 | uc001aqm.3 |
| | | | | | 9990456 | | | |
| MAATS1 | 609910 | NA | NA | NA | NA | NA | NA | NA |
| MACC1 | 612646 | NA | NA | NA | NA | 1643.98 | −35.055769 | uc003sus.4 |
| MACF1 | 608271 | 671.633 | −4.536509 | uc021olt.1 | chr1: 39951467- | 503.573 | −5.333482 | uc010oit.1 |
| | | | | | 39952810 | | | |
| MAD2L1BP | NA | NA | NA | NA | NA | NA | NA | NA |
| MAEL | 611368 | NA | NA | NA | NA | NA | NA | NA |
| MAF | 177075 | 1281.56 | −13.878332 | uc002ffm.3 | chr16: 79627745- | NA | NA | NA |
| | | | | | 79628356 | | | |
| MAGEA10 | 300343 | 454.317 | −7.361481 | uc004ffk.3 | chrX: 151301783- | NA | NA | NA |
| | | | | | 151302982 | | | |
| MAGEB1 | 300097 | 2067.34 | −20.338829 | uc004dcc.3 | chrX: 30269655- | 682.668 | −5.030571 | uc004dcc.3 |
| | | | | | 30270155 | | | |
| MAGEB18 | NA | 542.598 | −4.196924 | uc004dbq.2 | chrX: 26158297- | 522.629 | −7.759387 | uc004dbq.2 |
| | | | | | 26158853 | | | |
| MAGEB2 | 300098 | NA | NA | NA | NA | NA | NA | NA |
| MAGED1 | 300224 | 572.23 | −9.189668 | uc004dpm.3 | chrX: 51645242- | NA | NA | NA |
| | | | | | 51645450 | | | |
| MAGEE1 | 300759 | NA | NA | NA | NA | NA | NA | NA |
| MAGI3 | 615943 | 565.328 | −10.072857 | uc001edi.4 | chr1: 114224884- | NA | NA | NA |
| | | | | | 114228545 | | | |
| MAGOH | 602603 | 1424.77 | −8.698107 | uc001cvf.2 | chr1: 53692564- | NA | NA | NA |
| | | | | | 53692716 | | | |
| MAGOHB | NA | 789.542 | −7.938494 | uc001qyq.2 | chr12: 10756364- | 1536.74 | −15.001684 | uc001qyq.2 |
| | | | | | 10758873 | | | |
| MAK16 | NA | NA | NA | NA | NA | NA | NA | NA |
| MAL2 | 609684 | NA | NA | NA | NA | NA | NA | NA |
| MALT1 | 604860 | NA | NA | NA | NA | NA | NA | NA |
| MAN1A2 | 604345 | 275.216 | −4.333211 | uc001ehd.1 | chr1: 118065580- | 1066.54 | −7.955933 | uc001ehd.1 |
| | | | | | 118068320 | | | |
| MAN1B1 | 604346 | 437.606 | −5.496767 | uc004clf.1 | chr9: 140003044- | 292.407 | −4.573173 | uc004clf.1 |
| | | | | | 140003639 | | | |
| MAN2A1 | 154582 | NA | NA | NA | NA | 707.917 | −8.173541 | uc003kou.1 |
| MANEA | 612327 | 1716.62 | −22.033931 | uc003pon.3 | chr6: 96034913- | NA | NA | NA |
| | | | | | 96035254 | | | |
| MANSC4 | NA | NA | NA | NA | NA | NA | NA | NA |
| MAOB | 309860 | 1128.91 | −8.198852 | uc011mkx.2 | chrX: 43625857- | NA | NA | NA |
| | | | | | 43626865 | | | |
| MAP1LC3C | 609605 | NA | NA | NA | NA | NA | NA | NA |
| MAP2 | 157130 | NA | NA | NA | NA | 1346.2 | −8.246494 | uc002vdd.1 |
| MAP2K6 | 601254 | NA | NA | NA | NA | NA | NA | NA |
| MAP2K7 | 603014 | NA | NA | NA | NA | NA | NA | NA |
| MAP3K12 | 600447 | 360.324 | −7.292087 | uc001sdm.2 | chr12: 53874276- | 998.826 | −9.210452 | uc001sdm.2 |
| | | | | | 53874965 | | | |
| MAP3K15 | 300820 | NA | NA | NA | NA | 356.93 | −8.044757 | uc004czi.2 |
| MAP3K5 | 602448 | 486.085 | −5.866681 | uc011edj.2 | chr6: 136878187- | NA | NA | NA |
| | | | | | 136878895 | | | |
| MAP4K1 | 601983 | 312.683 | −4.035908 | uc002oix.1 | chr19: 39078281- | NA | NA | NA |
| | | | | | 39078449 | | | |
| MAP6 | 601783 | NA | NA | NA | NA | 666.594 | −4.290649 | uc001owv.3 |
| MAP7D1 | NA | NA | NA | NA | NA | 526.74 | −6.039878 | uc001bzz.3 |
| MAP7D2 | NA | 348.522 | −6.155233 | uc004czq.2 | chrX: 20024831- | NA | NA | NA |
| | | | | | 20026516 | | | |
| MAP7D3 | 300930 | 473.287 | −5.212678 | uc004ezt.3 | chrX: 135298974- | NA | NA | NA |
| | | | | | 135300652 | | | |
| MAP9 | 610070 | NA | NA | NA | NA | 700.774 | −12.215267 | uc003ios.3 |
| MAPK14 | 600289 | 513.672 | −2.297988 | uc011dth.2 | chr6: 36063853- | NA | NA | NA |
| | | | | | 36065831 | | | |
| MAPK6 | 602904 | 1749.78 | −13.789411 | uc002abp.3 | chr15: 52357198- | NA | NA | NA |
| | | | | | 52358462 | | | |
| MAPK | 601158 | NA | NA | NA | NA | 698.904 | −9.996503 | uc001jgo.3 |
| MAPK8IP2 | 607755 | NA | NA | NA | NA | NA | NA | NA |
| MAPK9 | 602896 | NA | NA | NA | NA | 516.706 | −5.867576 | uc003mls.4 |
| MARK1 | 606511 | NA | NA | NA | NA | 433.721 | −7.988182 | uc001hmm.4 |
| MARK4 | 606495 | 910.444 | −5.723791 | uc002pbb.2 | chr19: 45805969- | NA | NA | NA |
| | | | | | 45808541 | | | |
| MARS | 156560 | NA | NA | NA | NA | NA | NA | NA |
| MARS2 | 609728 | 994.117 | −9.502248 | uc002uuq.3 | chr2: 198571912- | NA | NA | NA |
| | | | | | 198573114 | | | |
| MARVELD2 | 610572 | 805.404 | −8.527594 | uc003jwq.3 | chr5: 68737482- | 990.996 | −8.80008 | uc003jwq.3 |
| | | | | | 68737890 | | | |
| MAS1 | 165180 | NA | NA | NA | NA | NA | NA | NA |
| MAST3 | 612258 | NA | NA | NA | NA | 293.17 | −7.930334 | uc002nhz.4 |
| MAST4 | NA | NA | NA | NA | NA | 1168.11 | −9.937645 | uc003jus.3 |
| MAT1A | 610550 | NA | NA | NA | NA | 243.121 | −4.775867 | uc001kbw.3 |
| MAT2A | 601468 | NA | NA | NA | NA | 1537.17 | −12.278736 | uc002spr.3 |
| MATN1 | 115437 | NA | NA | NA | NA | 404.029 | −5.703427 | uc001brz.3 |
| MATN2 | 602108 | NA | NA | NA | NA | 846.521 | −7.990106 | uc003yib.1 |
| MATN3 | 602109 | 218.059 | −2.353903 | uc002rdl.3 | chr2: 20191813- | NA | NA | NA |
| | | | | | 20192871 | | | |
| MATR3 | 164015 | NA | NA | NA | NA | NA | NA | NA |
| MBD1 | 156535 | NA | NA | NA | NA | NA | NA | NA |
| MBD2 | 603547 | NA | NA | NA | NA | 219.801 | −8.741468 | uc002lfh.2 |
| MBD5 | 611472 | NA | NA | NA | NA | 1743.63 | −11.598938 | uc002twm.4 |
| MBIP | 609431 | 944.464 | −13.231176 | uc001wtm.2 | chr14: 36767764- | 1474.81 | −1.562794 | uc001wtm.2 |
| | | | | | 36768288 | | | |
| MBL2 | 154545 | 593.228 | −10−598525 | uc001jjt.3 | chr10: 54525140- | NA | NA | NA |
| | | | | | 54527896 | | | |
| MBNL2 | 607327 | 564.104 | −9.09886 | uc001vnc.3 | chr13: 98009994- | NA | NA | NA |
| | | | | | 98011528 | | | |
| MBNL3 | 300413 | 903.775 | −10.787246 | uc004ewt.3 | chrX: 131503343- | NA | NA | NA |
| | | | | | 131513693 | | | |
| MC1R | 155555 | NA | NA | NA | NA | 523.001 | −8.234059 | uc002fpe.4 |
| MC5R | 600042 | 749.627 | −5.616246 | uc010xaf.2 | chr18: 13826743- | 1012.08 | −8.299275 | uc010xaf.2 |
| | | | | | 13826861 | | | |
| MCC | 159350 | 571.478 | −5.654323 | uc003kqj.4 | chr5: 112357796- | NA | NA | NA |
| | | | | | 112362998 | | | |
| MCCC2 | 609014 | NA | NA | NA | NA | NA | NA | NA |
| MCF2 | 311030 | NA | NA | NA | NA | 316.908 | −4.583737 | uc010nsh.2 |
| MCFD2 | 607788 | 388.014 | −6.330978 | uc002rvk.3 | chr2: 47129009- | 857.492 | −7.521137 | uc002rvk.3 |
| | | | | | 47132601 | | | |
| MCHR2 | 606111 | 1287.28 | −9.854367 | uc003pqh.1 | chr6: 100367786- | NA | NA | NA |
| | | | | | 100368815 | | | |
| MCM2 | 116945 | NA | NA | NA | NA | NA | NA | NA |
| MCM3AP | 603294 | NA | NA | NA | NA | NA | NA | NA |
| MCMBP | 610909 | NA | NA | NA | NA | NA | NA | NA |
| MCMDC2 | NA | NA | NA | NA | NA | NA | NA | NA |
| MCOLN3 | 607400 | NA | NA | NA | NA | 303.777 | −3.295331 | uc001dko.3 |
| MCPH1 | 607117 | 382.658 | −4.810288 | uc003wqh.3 | chr8: 6303077- | NA | NA | NA |
| | | | | | 6304940 | | | |
| MCRS1 | 609504 | 215.469 | −4.933746 | uc001rui.1 | chr12: 49952086- | NA | NA | NA |
| | | | | | 49952425 | | | |
| MCTP1 | 616296 | NA | NA | NA | NA | 856.491 | −7.204207 | uc003kku.2 |
| MCTS1 | 300587 | NA | NA | NA | NA | 815.102 | −7.648517 | uc004esx.3 |
| MDC1 | 607593 | NA | NA | NA | NA | 676.694 | −6.785601 | uc003nrf.4 |
| MDFIC | 614511 | 1139.39 | −14.446601 | uc022akf.1 | chr7: 114563552- | 391.223 | −5.492857 | uc022akf.1 |
| | | | | | 114563720 | | | |
| MDM1 | 613813 | 319.328 | −5.490174 | uc001sua.4 | chr12: 68718785- | 578.578 | −11.738192 | uc001sua.4 |
| | | | | | 68720265 | | | |
| MECOM | 165215 | NA | NA | NA | NA | NA | NA | NA |
| MECP2 | 300005 | 519.209 | −4.783117 | uc004fjv.2 | chrX: 153287264- | NA | NA | NA |
| | | | | | 153295817 | | | |
| MED12 | 300188 | NA | NA | NA | NA | NA | NA | NA |
| MED17 | 603810 | 1007.95 | −6.086082 | uc001pel.2 | chr11: 93521355- | NA | NA | NA |
| | | | | | 93522962 | | | |
| MED19 | 612385 | NA | NA | NA | NA | NA | NA | NA |
| MED22 | 185641 | 757.393 | −4.525297 | uc004cdc.3 | chr9: 136207755- | NA | NA | NA |
| | | | | | 136208354 | | | |
| MED28 | 610311 | 482.834 | −6.449887 | uc003gpi.1 | chr4: 17625422- | NA | NA | NA |
| | | | | | 17626160 | | | |
| MED30 | 610237 | NA | NA | NA | NA | 1244.27 | −9.722781 | uc003yoj.3 |
| MED31 | NA | NA | NA | NA | NA | NA | NA | NA |
| MED6 | 602984 | 695.616 | −5.388268 | uc001xmf.3 | chr14: 71050957- | 542.917 | −2.642949 | uc001xmf.3 |
| | | | | | 71051529 | | | |
| MED7 | 605045 | 1413.3 | −11.947574 | uc010jik.3 | chr5: 156565451- | NA | NA | NA |
| | | | | | 156565740 | | | |
| MEDAG | NA | NA | NA | NA | NA | NA | NA | NA |
| MEF2A | 600660 | 475.331 | −6.562262 | uc002bve.3 | chr15: 100252977- | 772.574 | −5.917129 | uc002bve.3 |
| | | | | | 100256629 | | | |
| MEF2C | 600662 | NA | NA | NA | NA | NA | NA | NA |
| MEGF10 | 612453 | NA | NA | NA | NA | NA | NA | NA |
| MEGF9 | 604268 | 540.67 | −6.056652 | uc004bkk.4 | chr9: 123420280- | 385.551 | −4.507734 | uc004bkk.4 |
| | | | | | 123421647 | | | |
| MEIG1 | 614174 | 1179.29 | −22.694091 | uc009xjk.1 | chr10: 15014641- | 673.893 | −6.549788 | uc009xjk.1 |
| | | | | | 15014850 | | | |
| MEIS3P1 | NA | NA | NA | NA | NA | NA | NA | NA |
| MEP1A | 600388 | NA | NA | NA | NA | NA | NA | NA |
| MERTK | 604705 | NA | NA | NA | NA | 1140.46 | −10.934985 | uc002thk.1 |
| MEST | 601029 | NA | NA | NA | NA | 209.448 | −6.092604 | uc003vqc.3 |
| METAP1 | 610151 | NA | NA | NA | NA | 936.696 | −9.100486 | uc003huf.4 |
| METAP1D | 610267 | NA | NA | NA | NA | NA | NA | NA |
| METAP2 | 601870 | NA | NA | NA | NA | 604.145 | −5.064142 | uc001tec.3 |
| METTL10 | NA | NA | NA | NA | NA | 471.345 | −2.757144 | uc001lhy.1 |
| METTL12 | NA | NA | NA | NA | NA | 1071.71 | −12.883165 | uc001nug.1 |
| METTL14 | 616504 | 568.4 | −6.121731 | uc003icf.3 | chr4: 119631458- | NA | NA | NA |
| | | | | | 119632077 | | | |
| METTL15 | NA | 591.836 | −13.360446 | uc001msg.2 | chr11: 28351943- | 942.759 | −14.555765 | uc001msg.2 |
| | | | | | 28355054 | | | |
| METTL16 | NA | NA | NA | NA | NA | NA | NA | NA |
| METTL21A | 615257 | 981.441 | −9.255038 | uc002vcf.2 | chr2: 208473839- | NA | NA | NA |
| | | | | | 208477769 | | | |
| METTL21B | 615258 | NA | NA | NA | NA | 821.991 | −8.075247 | uc001sqg.3 |
| METTL2A | NA | NA | NA | NA | NA | 303.709 | −6.906296 | uc002izv.2 |
| METTL3 | 612472 | NA | NA | NA | NA | 1394.42 | −15.469295 | uc010tlx.2 |
| METTL4 | NA | 952.524 | −10.6231 | uc010dkj.3 | chr18: 2537524- | 351.67 | −5.605668 | uc010dkj.3 |
| | | | | | 2539135 | | | |
| METTL6 | NA | NA | NA | NA | NA | 401.029 | −7.099132 | uc003bzt.1 |
| METTL8 | 609525 | 424.279 | −3.444104 | uc002ugs.4 | chr2: 172173913- | 201.44 | −4.659142 | uc002ugs.4 |
| | | | | | 172180872 | | | |
| METTL9 | 609388 | 207.345 | −5.027447 | uc002dje.3 | chr16: 21666754- | NA | NA | NA |
| | | | | | 21668792 | | | |
| MEX3C | 611005 | NA | NA | NA | NA | NA | NA | NA |
| MFAP1 | 600215 | NA | NA | NA | NA | 1093.51 | −9.341261 | uc001zth.1 |
| MFAP3L | 616523 | NA | NA | NA | NA | NA | NA | NA |
| MFHAS1 | 605352 | NA | NA | NA | NA | 714.464 | −6.099793 | uc003wsj.1 |
| MFN1 | 608506 | NA | NA | NA | NA | NA | NA | NA |
| MFSD8 | 611124 | NA | NA | NA | NA | 1550.15 | −12.313093 | uc003ifp.3 |
| MGARP | NA | 260.866 | −4.875288 | uc003ihr.1 | chr4: 140187317- | 969.434 | −12.373779 | uc003ihr.1 |
| | | | | | 140187752 | | | |
| MGAT2 | 602616 | NA | NA | NA | NA | 667.183 | −8.025358 | uc001wwr.3 |
| MGAT3 | 604621 | NA | NA | NA | NA | NA | NA | NA |
| MGAT4A | 604623 | NA | NA | NA | NA | 705.514 | −10.38458 | uc002sze.3 |
| MGAT5B | 612441 | NA | NA | NA | NA | 581.839 | −5.932271 | uc002jth.3 |
| MGME1 | 615076 | NA | NA | NA | NA | 459.062 | −5.54896 | uc002wqh.3 |
| MGST1 | 138330 | NA | NA | NA | NA | 1486.55 | −8.429703 | uc001rdf.3 |
| MGST2 | 601733 | 608.664 | −2.31517 | uc021xse.1 | chr4: 140625170- | 1095.09 | −5.35259 | uc021xse.1 |
| | | | | | 140625406 | | | |
| MIA2 | 608001 | NA | NA | NA | NA | NA | NA | NA |
| MICAL3 | 608882 | NA | NA | NA | NA | 623.456 | −5.541424 | uc002znh.2 |
| MICB | 602436 | NA | NA | NA | NA | 758.458 | −5.514129 | uc011fcy.2 |
| MICU2 | 610632 | NA | NA | NA | NA | 566.563 | −8.086002 | uc010tct.2 |
| MIEF1 | 615497 | NA | NA | NA | NA | 841.004 | −10.385107 | uc003axx.3 |
| MIEN1 | 611802 | NA | NA | NA | NA | NA | NA | NA |
| MIER1 | NA | 782.231 | −5.869119 | uc001ddc.2 | chr1: 67452157- | 712.907 | −7.235581 | uc001ddc.2 |
| | | | | | 67454302 | | | |
| MINK1 | 609426 | 390.782 | −5.78778 | uc010vsk.2 | chr17: 4800583- | 631.388 | −5.552811 | uc010vsk.2 |
| | | | | | 4801356 | | | |
| MINPP1 | 605391 | 229.672 | −5.468491 | uc001kev.3 | chr10: 89311943- | NA | NA | NA |
| | | | | | 89313218 | | | |
| MIOS | 615359 | NA | NA | NA | NA | NA | NA | NA |
| MIPEP | 602241 | NA | NA | NA | NA | 378.559 | −5.92779 | uc001uox.4 |
| MIS18A | NA | 218.187 | −5.673029 | uc002ypi.3 | chr21: 33640530- | NA | NA | NA |
| | | | | | 33641347 | | | |
| MIS18BP1 | NA | NA | NA | NA | NA | 1157.21 | −11.056193 | uc001wwf.3 |
| MITD1 | NA | NA | NA | NA | NA | NA | NA | NA |
| MITF | 156845 | 583.2 | −9.241189 | uc003dnz.3 | chr3: 70014400- | NA | NA | NA |
| | | | | | 70017488 | | | |
| MKI67 | 176741 | NA | NA | NA | NA | NA | NA | NA |
| MKLN1 | 605623 | 661.777 | −12.494789 | uc011kpl.2 | chr7: 131172488- | NA | NA | NA |
| | | | | | 131181398 | | | |
| MKRN1 | 607754 | NA | NA | NA | NA | 348.322 | −5.080409 | uc003vvu.4 |
| MKRN2OS | NA | NA | NA | NA | NA | 441.644 | −4.616069 | uc021wtc.1 |
| MKS1 | 609883 | 768.772 | −9.434113 | uc002ivr.2 | chr17: 56282797- | NA | NA | NA |
| | | | | | 56283439 | | | |
| MKX | 601332 | 855.782 | −5.977701 | uc001itx.4 | chr10: 27961803- | NA | NA | NA |
| | | | | | 27964157 | | | |
| MLEC | 613802 | 344.14 | −4.013345 | uc001tyy.1 | chr12: 121134349- | NA | NA | NA |
| | | | | | 121139667 | | | |
| MLF1 | 601402 | 1055.4 | −14.703596 | uc003fbx.3 | chr3: 158322992- | NA | NA | NA |
| | | | | | 158324249 | | | |
| MLH1 | 120436 | NA | NA | NA | NA | 854.716 | 0.166689 | uc003cgl.3 |
| MLLT10 | 602409 | NA | NA | NA | NA | NA | NA | NA |
| MLLT11 | 604684 | 964.857 | −7.940541 | uc001ewq.3 | chr1: 151039974- | NA | NA | NA |
| | | | | | 151040973 | | | |
| MLLT3 | 159558 | 1121.3 | −14.728143 | uc011lne.1 | chr9: 20344968- | NA | NA | NA |
| | | | | | 20346440 | | | |
| MLLT4 | 159559 | 447.903 | −6.204244 | uc021zij.1 | chr6: 168370616- | NA | NA | NA |
| | | | | | 168372700 | | | |
| MMAA | 607481 | NA | NA | NA | NA | NA | NA | NA |
| MMACHC | 609831 | 393.3 | −4.323811 | uc009vxv.3 | chr1: 45974888- | NA | NA | NA |
| | | | | | 45976739 | | | |
| MMADHC | 611935 | NA | NA | NA | NA | 1101.03 | −12.193309 | uc002txc.3 |
| MMD | 604467 | NA | NA | NA | NA | 1156.59 | −9.157682 | uc002iui.3 |
| MME | 120520 | 558.147 | −4.926569 | uc010hvr.1 | chr3: 154898249- | NA | NA | NA |
| | | | | | 154901518 | | | |
| MMGT1 | NA | NA | NA | NA | NA | NA | NA | NA |
| MMP10 | 185260 | NA | NA | NA | NA | NA | NA | NA |
| MMP15 | 602261 | NA | NA | NA | NA | 446.862 | −5.1118 | uc002ena.3 |
| MMP16 | 602262 | 1076.99 | −5.776699 | uc003yeb.4 | chr8: 89049460- | 664.669 | −5.144157 | uc003yeb.4 |
| | | | | | 89053688 | | | |
| MMP7 | 178990 | NA | NA | NA | NA | 1741.98 | −13.560378 | uc001phb.3 |
| MMP8 | 120355 | 648.809 | −5.895186 | uc001phe.2 | chr11: 102582526- | NA | NA | NA |
| | | | | | 102584078 | | | |
| MMRN1 | 601456 | 247.183 | −6.379406 | uc010iku.3 | chr4: 90874570- | NA | NA | NA |
| | | | | | 90875780 | | | |
| MMRN2 | 608925 | NA | NA | NA | NA | 368.154 | −6.446522 | uc001kea.3 |
| MMS22L | 615614 | NA | NA | NA | NA | 205.408 | −15.195844 | uc011eaf.2 |
| MOB1A | 609281 | 505.421 | −6.434412 | uc002skh.4 | chr2: 74383211- | NA | NA | NA |
| | | | | | 74383694 | | | |
| MOB1B | 609282 | 1700.59 | −1.890743 | uc003hfv.2 | chr4: 71841039- | 1879.17 | −23.993816 | uc003hfw.3 |
| | | | | | 71842460 | | | |
| MOB3C | NA | 486.778 | −7.809358 | uc001cqe.4 | chr1: 47073387- | NA | NA | NA |
| | | | | | 47075326 | | | |
| MOCS1 | 603707 | NA | NA | NA | NA | NA | NA | NA |
| MOCS2 | 603708 | 451.07 | −1.905312 | uc003joz.3 | chr5: 52393895- | 494.15 | −3.696608 | uc011cqf.2 |
| | | | | | 52394431 | | | |
| MON1A | 611464 | NA | NA | NA | NA | NA | NA | NA |
| MON1B | 608954 | NA | NA | NA | NA | 473.972 | −6.794786 | uc002fez.3 |
| MORC2 | 616661 | NA | NA | NA | NA | 251.393 | −6.209134 | uc003aje.1 |
| MORC4 | NA | 913.53 | −6.405782 | uc004emu.4 | chrX: 106183964- | 298.63 | −6.004877 | uc004emp.4 |
| | | | | | 106184708 | | | |
| MORF4L2 | 300409 | 702.689 | −9.502926 | uc004ekw.3 | chrX: 102930426- | NA | NA | NA |
| | | | | | 102931088 | | | |
| MOV10L1 | 605794 | NA | NA | NA | NA | NA | NA | NA |
| MPDU1 | 604041 | NA | NA | NA | NA | NA | NA | NA |
| MPEG1 | 610390 | 661.091 | −5.560352 | uc001nnu.4 | chr11: 58975983- | NA | NA | NA |
| | | | | | 58978187 | | | |
| MPHOSPH8 | 611626 | 500.404 | −2.170723 | uc001umg.3 | chr13: 20245127- | NA | NA | NA |
| | | | | | 20245258 | | | |
| MPP7 | 610973 | 726.408 | −10.223326 | uc001iua.1 | chr10: 28339923- | NA | NA | NA |
| | | | | | 28342993 | | | |
| MPPED2 | 600911 | NA | NA | NA | NA | 360.793 | −4.455897 | uc001msr.3 |
| MPRIP | 612935 | NA | NA | NA | NA | 801.615 | −8.110599 | uc002gqu.2 |
| MR1 | 600764 | NA | NA | NA | NA | 237.983 | −7.818823 | uc001goq.2 |
| MRAP | 609196 | NA | NA | NA | NA | 1180.47 | −7.67313 | uc002ypk.3 |
| MRE11A | 600814 | NA | NA | NA | NA | NA | NA | NA |
| MRGPRX2 | 607228 | NA | NA | NA | NA | NA | NA | NA |
| MRM1 | NA | 345.925 | −4.215284 | uc002hne.3 | chr17: 34964852- | NA | NA | NA |
| | | | | | 34965407 | | | |
| MROH7 | NA | 1241.5 | −8.289133 | uc001cxo.2 | chr1: 55129467- | 541.294 | −4.353334 | uc001cxo.2 |
| | | | | | 55129728 | | | |
| MROH9 | NA | 821.977 | −5.450956 | uc001ghg.3 | chr1: 170967542- | 239.452 | −5.36657 | uc001ghg.3 |
| | | | | | 170967946 | | | |
| MRPL11 | 611826 | 403.329 | −9.019962 | uc001ohy.4 | chr11: 66202550- | 339.352 | −3.672304 | uc001ohy.4 |
| | | | | | 66203583 | | | |
| MRPL19 | 611832 | NA | NA | NA | NA | NA | NA | NA |
| MRPL2 | 611822 | NA | NA | NA | NA | NA | NA | NA |
| MRPL22 | 611835 | NA | NA | NA | NA | 788.527 | −7.551554 | uc003lvy.4 |
| MRPL35 | 611841 | 948.259 | −9.989012 | uc002srg.4 | chr2: 86437792- | 382.741 | −8.248625 | uc002srf.4 |
| | | | | | 86440477 | | | |
| MRPL36 | 611842 | NA | NA | NA | NA | 471.574 | −9.90122 | uc003jcx.4 |
| MRPL42 | 611847 | 1361.77 | −14.748872 | uc001tcq.3 | chr12: 93894998- | NA | NA | NA |
| | | | | | 93897548 | | | |
| MRPL46 | 611851 | NA | NA | NA | NA | 210.812 | −5.674106 | uc002bmi.1 |
| MRPL48 | 611853 | NA | NA | NA | NA | NA | NA | NA |
| MRPL50 | 611854 | 439.895 | −6.598085 | uc004bbe.2 | chr9: 104152249- | NA | NA | NA |
| | | | | | 104152747 | | | |
| MRPL57 | 611997 | NA | NA | NA | NA | NA | NA | NA |
| MRPL9 | 611824 | 310.885 | −5.04767 | uc001eyv.3 | chr1: 151732123- | 473.687 | −4.261176 | uc001eyv.3 |
| | | | | | 151732525 | | | |
| MRPS14 | 611978 | NA | NA | NA | NA | NA | NA | NA |
| MRPS16 | 609204 | NA | NA | NA | NA | NA | NA | NA |
| MRPS18C | 611983 | NA | NA | NA | NA | NA | NA | NA |
| MRPS23 | 611985 | 1187.2 | −5.637216 | uc002ivc.3 | chr17: 55916287- | NA | NA | NA |
| | | | | | 55917143 | | | |
| MRPS33 | 611993 | NA | NA | NA | NA | 722.865 | −8.957283 | uc003vwd.4 |
| MRPS36 | 611996 | NA | NA | NA | NA | 450.952 | −6.919258 | uc003jvq.3 |
| MRPS5 | 611972 | 823.59 | −3.925327 | uc002sub.3 | chr2: 95752952- | NA | NA | NA |
| | | | | | 95753101 | | | |
| MRPS6 | 611973 | 216.538 | −8.89253 | uc002ytp.2 | chr21: 35514901- | 501.207 | −7.396125 | uc002ytp.2 |
| | | | | | 35515334 | | | |
| MRS2 | NA | 689.93 | −7.005284 | uc003nea.3 | chr6: 24423285- | 621.06 | −8.05039 | uc003neb.3 |
| | | | | | 24423705 | | | |
| MS4A1 | 112210 | NA | NA | NA | NA | NA | NA | NA |
| MS4A13 | NA | NA | NA | NA | NA | 360.008 | −5.178053 | uc001nps.3 |
| MS4A2 | 147138 | NA | NA | NA | NA | NA | NA | NA |
| MS4A4A | 606547 | 886.209 | −13.381437 | uc001noz.3 | chr11: 60075652- | NA | NA | NA |
| | | | | | 60076445 | | | |
| MS4A6A | 606548 | NA | NA | NA | NA | 1036.46 | −4.782558 | uc001noq.3 |
| MSANTD3 | NA | 269.782 | −4.624739 | uc022bkz.1 | chr9: 103212839- | NA | NA | NA |
| | | | | | 103214016 | | | |
| MSH4 | 602105 | NA | NA | NA | NA | 958.946 | −8.580759 | uc001dhd.2 |
| MSL | 614801 | 1391.75 | −12.745392 | uc002hua.4 | chr17: 38290623- | NA | NA | NA |
| | | | | | 38293045 | | | |
| MSL2 | 614802 | 1023.52 | −6.773401 | uc011bmb.1 | chr3: 135867760- | 750.899 | −6.435021 | uc011bmb.1 |
| | | | | | 135869988 | | | |
| MSL3 | 300609 | NA | NA | NA | NA | NA | NA | NA |
| MSRA | 601250 | 1284.16 | −10.315689 | uc003wsx.3 | chr8: 10285823- | NA | NA | NA |
| | | | | | 10286401 | | | |
| MSX1 | 142983 | NA | NA | NA | NA | 233.526 | −4.206923 | uc003gif.3 |
| MT1A | 156350 | 653.041 | −6.786994 | uc002ejq.3 | chr16: 56673863- | NA | NA | NA |
| | | | | | 56673999 | | | |
| MT1E | 156351 | NA | NA | NA | NA | 367.733 | −6.279567 | uc002ejl.3 |
| MT1F | 156352 | 682.24 | −6.698985 | uc002ejt.3 | chr16: 56693077- | NA | NA | NA |
| | | | | | 56693215 | | | |
| MT1G | 156353 | NA | NA | NA | NA | 760.054 | −6.531752 | uc002eju.1 |
| MT1H | 156354 | NA | NA | NA | NA | 533.954 | −6.704343 | uc002ejw.3 |
| MT1M | 156357 | NA | NA | NA | NA | 1252.53 | −5.421104 | uc002ejn.3 |
| MT1X | 156359 | NA | NA | NA | NA | 1008.58 | −12.130377 | uc002ejy.3 |
| MT2A | 156360 | 608.797 | −3.913272 | uc002ejh.3 | chr16: 56643267- | 672.533 | −10.058801 | uc002ejh.3 |
| | | | | | 56643409 | | | |
| MTCP1 | 300116 | NA | NA | NA | NA | 786.117 | −8.124339 | uc004fmz.2 |
| MTDH | 610323 | NA | NA | NA | NA | 442.372 | −6.234994 | uc003yhz.3 |
| MTERF2 | NA | 474.639 | −4.529601 | uc001tme.1 | chr12: 107371069- | NA | NA | NA |
| | | | | | 107371334 | | | |
| MTF2 | 609882 | NA | NA | NA | NA | 293.491 | −6.462448 | uc001dpi.4 |
| MTFMT | 611766 | 633.728 | −9.755785 | uc002aof.4 | chr15: 65293850- | NA | NA | NA |
| | | | | | 65295399 | | | |
| MTFR1 | NA | NA | NA | NA | NA | 1031.05 | −13.498363 | uc011lep.2 |
| MTG2 | 610919 | NA | NA | NA | NA | 572.866 | −5.523039 | uc002yce.4 |
| MTHFD2 | 604887 | 575.091 | −6.025016 | uc002skj.3 | chr2: 74441370- | NA | NA | NA |
| | | | | | 74442424 | | | |
| MTHFD2L | 614047 | NA | NA | NA | NA | 1912.15 | −17.854173 | uc011cbj.2 |
| MTHFR | 607093 | 524.224 | −5.1118 | uc001atb.1 | chr1: 11845787- | NA | NA | NA |
| | | | | | 11850736 | | | |
| MTHFS | 604197 | NA | NA | NA | NA | NA | NA | NA |
| MTMR1 | 300171 | NA | NA | NA | NA | NA | NA | NA |
| MTMR11 | NA | 776.707 | −1.083355 | uc001etm.2 | chr1: 149900543- | 1069.18 | −8.667684 | uc001eti.4 |
| | | | | | 149900758 | | | |
| MTMR14 | 611089 | NA | NA | NA | NA | NA | NA | NA |
| MTMR6 | 603561 | 645.7 | −5.381745 | uc001uqe.1 | chr13: 25802307- | NA | NA | NA |
| | | | | | 25802677 | | | |
| MTMR8 | NA | 401.35 | −4.992822 | uc004dvs.3 | chrX: 63487961- | NA | NA | NA |
| | | | | | 63488416 | | | |
| MTMR9 | 606260 | NA | NA | NA | NA | 599.944 | −5.585691 | uc003wtm.3 |
| MTOR | 601231 | NA | NA | NA | NA | NA | NA | NA |
| MTR | 156570 | NA | NA | NA | NA | 859.695 | −1.609293 | uc001hyi.4 |
| MTRF1L | 613542 | 873.361 | −5.774239 | uc003qpi.4 | chr6: 153308400- | NA | NA | NA |
| | | | | | 153311029 | | | |
| MTRNR2L10 | NA | 483.947 | −5.771478 | uc022bxr.1 | chrX: 55207824- | NA | NA | NA |
| | | | | | 55208352 | | | |
| MTX2 | 608555 | 954.446 | −7.320755 | uc002ukw.3 | chr2: 177202393- | NA | NA | NA |
| | | | | | 177202753 | | | |
| MUC15 | 608566 | NA | NA | NA | NA | 389.198 | −8.549453 | uc001mqw.3 |
| MUC2 | NA | NA | NA | NA | NA | NA | NA | NA |
| MUCL1 | 610857 | 1378.95 | −7.484818 | uc001sgk.3 | chr12: 55252027- | NA | NA | NA |
| | | | | | 55252174 | | | |
| MUL1 | 612037 | NA | NA | NA | NA | NA | NA | NA |
| MURC | NA | 389.119 | −7.595946 | uc004bba.3 | chr9: 103348734- | NA | NA | NA |
| | | | | | 103350173 | | | |
| MUS81 | 606591 | 280.923 | −6.049928 | uc001ofv.4 | chr11: 65633524- | NA | NA | NA |
| | | | | | 65633914 | | | |
| MUTYH | 604933 | NA | NA | NA | NA | 719 | −7.380917 | uc001cnf.3 |
| MVK | 251170 | 586.893 | −6.492262 | uc001toy.4 | chr12: 110034383- | NA | NA | NA |
| | | | | | 110035071 | | | |
| MXRA7 | NA | NA | NA | NA | NA | 666.856 | −7.585167 | uc002jsk.1 |
| MYB | 189990 | NA | NA | NA | NA | NA | NA | NA |
| MYBBP1A | 604885 | 497.383 | 0.368133 | uc002fya.4 | chr17: 4442191- | 651.053 | −3.161552 | uc002fya.4 |
| | | | | | 4442709 | | | |
| MYCT1 | NA | 1304.83 | −10.636237 | uc003qpc.4 | chr6: 153043389- | NA | NA | NA |
| | | | | | 153045715 | | | |
| MYD88 | 602170 | NA | NA | NA | NA | NA | NA | NA |
| MYEF2 | NA | NA | NA | NA | NA | NA | NA | NA |
| MYF5 | 159990 | 357.21 | −4.402231 | uc001szg.2 | chr12: 81112831- | NA | NA | NA |
| | | | | | 81113447 | | | |
| MYH3 | 160720 | 375.941 | −6.907549 | uc002gmq.2 | chr17: 10531843- | NA | NA | NA |
| | | | | | 10531969 | | | |
| MYL1 | 160780 | NA | NA | NA | NA | NA | NA | NA |
| MYL12A | NA | NA | NA | NA | NA | 732.759 | −5.462073 | uc002klr.3 |
| MYLIP | 610082 | 493.276 | −6.278528 | uc003nbq.3 | chr6: 16146983- | 801.184 | −10.347252 | uc003nbq.3 |
| | | | | | 16148478 | | | |
| MYLK3 | 612147 | NA | NA | NA | NA | 847.911 | −7.544139 | uc002eei.4 |
| MYLK4 | NA | NA | NA | NA | NA | NA | NA | NA |
| MYO1B | 606537 | NA | NA | NA | NA | 1290.44 | −11.485266 | uc002usq.2 |
| MYO3B | 610040 | NA | NA | NA | NA | 422.462 | −9.185753 | uc002uga.3 |
| MYO9A | 604875 | NA | NA | NA | NA | NA | NA | NA |
| MYOM2 | 603509 | NA | NA | NA | NA | 451.193 | −6.457045 | uc003wpx.4 |
| MYOT | 604103 | NA | NA | NA | NA | 750.043 | −9.755893 | uc011cye.2 |
| MYOZ2 | 605602 | NA | NA | NA | NA | NA | NA | NA |
| MYZAP | 614071 | 207.5 | −4.692024 | uc002aei.3 | chr15: 57976697- | NA | NA | NA |
| | | | | | 57977562 | | | |
| N6AMT2 | NA | NA | NA | NA | NA | NA | NA | NA |
| NBEAP1 | NA | NA | NA | NA | NA | NA | NA | NA |
| NBN | 602667 | NA | NA | NA | NA | 697.897 | −7.547016 | uc011lgb.1 |
| NCAM2 | 602040 | 915.155 | −7.1916 | uc002yld.2 | chr21: 22910279- | NA | NA | NA |
| | | | | | 22912517 | | | |
| NCAPG2 | 608532 | 688.189 | −9.129682 | uc011kwc.1 | chr7: 158424003- | NA | NA | NA |
| | | | | | 158424357 | | | |
| NCBP1 | 600469 | 742.559 | −5.689753 | uc004axq.3 | chr9: 100433482- | NA | NA | NA |
| | | | | | 100436029 | | | |
| NCF1 | 608512 | NA | NA | NA | NA | 897.082 | −4.745404 | uc011kfh.1 |
| NCF2 | 608515 | NA | NA | NA | NA | NA | NA | NA |
| NCOA7 | 609752 | 274.779 | −4.133205 | uc003qag.3 | chr6: 126206494- | 236.364 | −5.953553 | uc003qae.4 |
| | | | | | 126207312 | | | |
| NDE1 | 609449 | NA | NA | NA | NA | 727.362 | −7.905564 | uc010uzy.2 |
| NDN | 602117 | 1118.32 | −13.646634 | uc001ywk.3 | chr15: 23930554- | NA | NA | NA |
| | | | | | 23931398 | | | |
| NDUFA1 | 300078 | 296.65 | −5.591052 | uc004esc.4 | chrX: 119010498- | NA | NA | NA |
| | | | | | 119010629 | | | |
| NDUFA13 | 609435 | 207.053 | −4.633806 | uc021uqv.1 | chr19: 19643913- | NA | NA | NA |
| | | | | | 19645953 | | | |
| NDUFA9 | 603834 | NA | NA | NA | NA | NA | NA | NA |
| NDUFAF2 | 609653 | 277.017 | −5.318741 | uc003jsp.4 | chr5: 60448783- | NA | NA | NA |
| | | | | | 60448864 | | | |
| NDUFAF4 | 611776 | NA | NA | NA | NA | 1754.71 | −19.460117 | uc003pow.3 |
| NDUFAF5 | 612360 | NA | NA | NA | NA | 295.892 | −11.994758 | uc002wom.3 |
| NDUFAF6 | 612392 | 898.906 | −7.426782 | uc003yhi.3 | chr8: 96070166- | NA | NA | NA |
| | | | | | 96070944 | | | |
| NDUFB10 | 603843 | NA | NA | NA | NA | 520.786 | −5.996149 | uc002cni.2 |
| NDUFB2 | 603838 | NA | NA | NA | NA | 290.157 | −9.524346 | uc003vwa.3 |
| NDUFB4 | 603840 | NA | NA | NA | NA | NA | NA | NA |
| NDUFB5 | 603841 | 499.048 | −6.025016 | uc003fkc.3 | chr3: 179341829- | 1236.51 | −1.159269 | uc003fkc.3 |
| | | | | | 179342288 | | | |
| NDUFC1 | 603844 | NA | NA | NA | NA | NA | NA | NA |
| NDUFS2 | 602985 | 1488.75 | −7.76299 | uc010pki.2 | chr1: 161180505- | NA | NA | NA |
| | | | | | 161180839 | | | |
| NDUFS3 | 603846 | NA | NA | NA | NA | 388.129 | −10.242056 | uc001nft.3 |
| NDUFS6 | 603848 | 296.026 | −6.411042 | uc003jcy.3 | chr5: 1816031- | NA | NA | NA |
| | | | | | 1816167 | | | |
| NEB | 161650 | NA | NA | NA | NA | 868.93 | −6.806883 | uc010zbz.2 |
| NEBL | 605491 | NA | NA | NA | NA | NA | NA | NA |
| NECAB1 | NA | 1389.45 | −14.575835 | uc011lgg.2 | chr8: 91967741- | NA | NA | NA |
| | | | | | 91971630 | | | |
| NEDD4 | 602278 | NA | NA | NA | NA | 1068.77 | −10.637458 | uc002adi.3 |
| NEDD4L | 606384 | 360.397 | −6.769277 | uc002lgx.3 | chr18: 56063502- | NA | NA | NA |
| | | | | | 56068772 | | | |
| NEDD9 | 602265 | NA | NA | NA | NA | 1310.41 | −7.909438 | uc003mzv.2 |
| NEFH | 162230 | NA | NA | NA | NA | 693.432 | −6.318629 | uc003afo.3 |
| NEIL2 | 608933 | NA | NA | NA | NA | 527.543 | −6.359267 | uc003wue.2 |
| NEIL3 | 608934 | NA | NA | NA | NA | NA | NA | NA |
| NEK1 | 604588 | 472.634 | −5.036316 | uc003isb.2 | chr4: 170314421- | 223.388 | −9.799785 | uc003isg.1 |
| | | | | | 170315660 | | | |
| NEK10 | NA | 264.579 | −6.816542 | uc003cds.1 | chr3: 27203757- | 991.122 | −5.166417 | uc003cdt.2 |
| | | | | | 27203937 | | | |
| NEK5 | NA | NA | NA | NA | NA | 830.366 | −7.89554 | uc001vge.3 |
| NEK8 | 609799 | 857.26 | −9.465329 | uc002hcp.3 | chr17: 27069006- | NA | NA | NA |
| | | | | | 27069784 | | | |
| NES | 600915 | NA | NA | NA | NA | NA | NA | NA |
| NEURL1 | 603804 | 562.573 | −5.888658 | uc001kxh.3 | chr10: 105350130- | NA | NA | NA |
| | | | | | 105352309 | | | |
| NEUROD4 | 611635 | NA | NA | NA | NA | 298.48 | −6.823691 | uc001sgp.4 |
| NEXN | 613121 | 1593.16 | −3.127126 | uc001dib.4 | chr1: 78408515- | 1120.88 | −9.127844 | uc001dib.4 |
| | | | | | 78409578 | | | |
| NF1 | 613113 | 575.535 | −6.286842 | uc002hgg.3 | chr17: 29701174- | 546.835 | −7.587151 | uc002hge.2 |
| | | | | | 29704695 | | | |
| NFASC | 609145 | NA | NA | NA | NA | 419.868 | −5.997229 | uc001hbi.3 |
| NFAT5 | 604708 | 1142.38 | −0.516336 | uc002exh.2 | chr16: 69718885- | 654.503 | −6.87873 | uc002exh.2 |
| | | | | | 69720072 | | | |
| NFATC4 | 602699 | NA | NA | NA | NA | 970.99 | −11.033481 | uc010tok.2 |
| NFE2L2 | 600492 | NA | NA | NA | NA | 747.312 | −6.286986 | uc010frb.3 |
| NFKBIA | 164008 | NA | NA | NA | NA | NA | NA | NA |
| NFU1 | 608100 | NA | NA | NA | NA | 499.988 | −8.597326 | uc002sfj.3 |
| NFYA | 189903 | NA | NA | NA | NA | NA | NA | NA |
| NGB | 605304 | NA | NA | NA | NA | 200.42 | −6.181124 | uc001xtg.1 |
| NGBN | 610777 | 918.406 | −11.041964 | uc001wjy.3 | chr14: 23947236- | 2151.34 | −10.601845 | uc001wjy.3 |
| | | | | | 23947402 | | | |
| NGEF | 605991 | NA | NA | NA | NA | 868.764 | −4.171809 | uc010zmm.1 |
| NGFRAP1 | 300361 | NA | NA | NA | NA | NA | NA | NA |
| NGLY1 | 610661 | 1362.2 | −2.452249 | uc003cdl.3 | chr3: 25760435- | 557.437 | −4.92067 | uc003cdl.3 |
| | | | | | 25760950 | | | |
| NGRN | NA | NA | NA | NA | NA | 342.663 | −6.394717 | uc002bpf.1 |
| NHP2L1 | NA | 347.473 | −10.379331 | uc003bav.3 | chr22: 42069937- | 1699.07 | −10.7621 | uc003bav.3 |
| | | | | | 42070936 | | | |
| NHSL1 | NA | NA | NA | NA | NA | 1000.45 | −6.626936 | uc011edp.2 |
| NID1 | 131390 | NA | NA | NA | NA | 207.408 | −4.418834 | uc009xgc.3 |
| NID2 | 605399 | NA | NA | NA | NA | NA | NA | NA |
| NIF3L1 | 605778 | 1515.62 | −8.26265 | uc002uwl.2 | chr2: 201768402- | NA | NA | NA |
| | | | | | 201768655 | | | |
| NIFK | 611970 | 1043.04 | −6.502793 | uc002tnk.3 | chr2: 122484521- | 829.152 | −10.6482 | uc002tnk.3 |
| | | | | | 122485299 | | | |
| NIN | 608684 | 256.08 | −7.783399 | uc001wyi.3 | chr14: 51186481- | NA | NA | NA |
| | | | | | 51190180 | | | |
| NINL | 609580 | NA | NA | NA | NA | 549.439 | −7.456744 | uc002wuw.1 |
| NIPAL2 | NA | 362.131 | −7.769169 | uc011lgw.1 | chr8: 99204387- | NA | NA | NA |
| | | | | | 99205205 | | | |
| NIT1 | 604618 | NA | NA | NA | NA | 832.893 | −5.039194 | uc001fxw.3 |
| NKAIN2 | 609758 | NA | NA | NA | NA | 616.187 | −5.165386 | uc003pzn.1 |
| NKAIN4 | 612873 | 377.758 | −4.055558 | uc002yek.3 | chr20: 61872136- | NA | NA | NA |
| | | | | | 61872848 | | | |
| NKAP | 300766 | NA | NA | NA | NA | 1214.92 | −6.978541 | uc004esg.3 |
| NKRF | 300440 | 289.183 | −8.111559 | uc004erq.3 | chrX: 118722300- | NA | NA | NA |
| | | | | | 118723314 | | | |
| NKTR | 161565 | NA | NA | NA | NA | NA | NA | NA |
| NLN | 611530 | 1354 | −12.06769 | uc003jue.3 | chr5: 65088780- | NA | NA | NA |
| | | | | | 65089459 | | | |
| NLRP2 | 609364 | 483.161 | −6.998254 | uc021vbq.1 | chr19:55512267- | NA | NA | NA |
| | | | | | 55512510 | | | |
| NLRP8 | 609659 | NA | NA | NA | NA | NA | NA | NA |
| NMB | 162340 | NA | NA | NA | NA | NA | NA | NA |
| NME5 | 603575 | NA | NA | NA | NA | NA | NA | NA |
| NME7 | 613465 | 202.328 | −6.379406 | uc001gfv.1 | chr1: 169252460- | NA | NA | NA |
| | | | | | 169256454 | | | |
| NME9 | NA | NA | NA | NA | NA | NA | NA | NA |
| NMI | 603525 | 1611.8 | −15.733976 | uc002txj.2 | chr2: 152133769- | 1497.24 | −9.074067 | uc002txj.2 |
| | | | | | 152135288 | | | |
| NMRK2 | 608705 | NA | NA | NA | NA | NA | NA | NA |
| NMT1 | 160993 | 548.174 | −7.265023 | uc002ihz.3 | chr17: 43183008- | NA | NA | NA |
| | | | | | 43186382 | | | |
| NMUR2 | 605108 | 282.172 | −5.560655 | uc003luv2 | chr5: 151771102- | NA | NA | NA |
| | | | | | 151771751 | | | |
| NOA1 | 614919 | 935.299 | −9.757211 | uc003hck.3 | chr4: 57829516- | NA | NA | NA |
| | | | | | 57829615 | | | |
| NOC3L | 610769 | NA | NA | NA | NA | 1076.97 | −8.583747 | uc001kjq.1 |
| NOD1 | 605980 | 377.586 | −4.231693 | uc003tav.3 | chr7: 30464143- | NA | NA | NA |
| | | | | | 30465253 | | | |
| NOD2 | 605956 | NA | NA | NA | NA | 297.952 | −5.237521 | uc002egm.1 |
| NOL4 | 603577 | 685.762 | −5.289737 | uc010xbs.2 | chr18: 31431064- | NA | NA | NA |
| | | | | | 31432805 | | | |
| NOL8 | 611534 | NA | NA | NA | NA | 515.589 | −8.229101 | uc022bjw.1 |
| NOP14 | 611526 | NA | NA | NA | NA | 441.727 | −5.163575 | uc003ggj.1 |
| NOS3 | 163729 | 589.319 | −9.373703 | uc011kuz.2 | chr7: 150700377- | 1725.64 | −3.686087 | uc003wif.3 |
| | | | | | 150701023 | | | |
| NOSTRIN | 607496 | 248.746 | −7.808149 | uc002uef.3 | chr2: 169721481- | NA | NA | NA |
| | | | | | 169721849 | | | |
| NOTCH1 | 190198 | 675.846 | −10.142011 | uc004cia.1 | chr9: 139396889- | NA | NA | NA |
| | | | | | 139397370 | | | |
| NOTCH2 | 600275 | 507.881 | −4.552759 | uc021osy.1 | chr1: 120492743- | 603.321 | −6.582401 | uc001eik.3 |
| | | | | | 120493116 | | | |
| NOTCH3 | 600276 | NA | NA | NA | NA | NA | NA | NA |
| NOX1 | 300225 | 1158.35 | −15.496887 | uc004egj.3 | chrX: 100098313- | NA | NA | NA |
| | | | | | 100098940 | | | |
| NOX5 | 606572 | 634.735 | −8.098001 | uc002arp.2 | chr15: 69349037- | NA | NA | NA |
| | | | | | 69349501 | | | |
| NOXRED1 | NA | 980.401 | −12.824805 | uc001xfr.3 | chr14: 77860365- | 1032.1 | −14.198376 | uc001xtr.3 |
| | | | | | 77860973 | | | |
| NPAS3 | 609430 | 254.242 | −5.631028 | uc001wrs.3 | chr14: 34270316- | NA | NA | NA |
| | | | | | 34273382 | | | |
| NPC1L1 | 608010 | 719.82 | −6.551273 | uc003tlb.3 | chr7: 44552135- | NA | NA | NA |
| | | | | | 44553045 | | | |
| NPEPPS | 606793 | NA | NA | NA | NA | NA | NA | NA |
| NPFFR2 | 607449 | NA | NA | NA | NA | NA | NA | NA |
| NPHS1 | 602716 | 407.701 | −5.305648 | uc002oby.3 | chr19: 36316274- | NA | NA | NA |
| | | | | | 36317415 | | | |
| NPL | 611412 | NA | NA | NA | NA | NA | NA | NA |
| NPM1 | 164040 | 470.122 | −9.842239 | uc003mbh.3 | chr5: 170833410- | NA | NA | NA |
| | | | | | 170833731 | | | |
| NPPB | 600295 | NA | NA | NA | NA | 852.976 | −10.35564 | uc001atj.3 |
| NPR3 | 108962 | NA | NA | NA | NA | NA | NA | NA |
| NQO1 | 125860 | NA | NA | NA | NA | NA | NA | NA |
| NR0B2 | 604630 | NA | NA | NA | NA | 218.439 | −4.98961 | uc001bnf.3 |
| NR1D2 | 602304 | 1546.02 | −15.663207 | uc003ccs.2 | chr3: 24018911- | NA | NA | NA |
| | | | | | 24022109 | | | |
| NR1H3 | 602423 | 249.665 | −4.766255 | uc009yll.2 | chr11: 47290248- | 700.573 | −5.846009 | uc009yll.2 |
| | | | | | 47290584 | | | |
| NR1H4 | 603826 | NA | NA | NA | NA | NA | NA | NA |
| NR1I3 | 603881 | 349.011 | −6.101502 | uc021pbw.1 | chr1: 161199456- | 317.564 | −3.9096 | uc021pbw.1 |
| | | | | | 161199586 | | | |
| NR2C2 | 601426 | NA | NA | NA | NA | 753.365 | −11.669092 | uc003bzi.3 |
| NR2C2AP | 608719 | NA | NA | NA | NA | NA | NA | NA |
| NR2F2 | 107773 | 700.642 | −6.888181 | uc002btp.3 | chr15: 96880852- | NA | NA | NA |
| | | | | | 96883492 | | | |
| NR5A2 | 604453 | 448.188 | −5.046123 | uc009wzh.3 | chr1: 200143339- | NA | NA | NA |
| | | | | | 200146550 | | | |
| NRCAM | 601581 | 1257.77 | −7.516446 | uc003vfa.3 | chr7: 107788071- | NA | NA | NA |
| | | | | | 107790354 | | | |
| NRG1 | 142445 | 677.651 | −5.568273 | uc022ats.1 | chr8: 32621921- | 713.441 | −3.864056 | uc003xip.3 |
| | | | | | 32622558 | | | |
| NRG2 | 603818 | NA | NA | NA | NA | NA | NA | NA |
| NRG3 | 605533 | NA | NA | NA | NA | 402.629 | −6.396274 | uc001kco.2 |
| NRG4 | 610894 | 569.593 | −10.781214 | uc002bbo.3 | chr15: 76234328- | NA | NA | NA |
| | | | | | 76235978 | | | |
| NRIP1 | 602490 | 588.216 | −5.947653 | uc002yjx.2 | chr21: 16333556- | NA | NA | NA |
| | | | | | 16337036 | | | |
| NRK | 300791 | 746.296 | −11.270158 | uc004emd.3 | chrX: 105199593- | NA | NA | NA |
| | | | | | 105202602 | | | |
| NRP1 | 602069 | 664.177 | −10.425385 | uc001iwv.4 | chr10: 33466419- | NA | NA | NA |
| | | | | | 33469003 | | | |
| NRP2 | 602070 | NA | NA | NA | NA | NA | NA | NA |
| NRTN | 602018 | NA | NA | NA | NA | 597.609 | −5.151089 | uc002mde.3 |
| NSD1 | 606681 | 434.03 | −4.613158 | uc003mft.4 | chr5: 176722461- | 2106.16 | −18.648121 | uc003mfq.3 |
| | | | | | 176727214 | | | |
| NSG1 | NA | 740.687 | −8.660488 | uc011bvz.2 | chr4: 4419163- | NA | NA | NA |
| | | | | | 4420785 | | | |
| NSL1 | 609174 | NA | NA | NA | NA | 663.154 | −6.690391 | uc001hjm.3 |
| NSMCE4A | 612987 | NA | NA | NA | NA | 447.329 | −4.923393 | uc001lfs.3 |
| NSUN2 | 610916 | 498.745 | −4.774669 | uc003jdt.3 | chr5: 6599352- | NA | NA | NA |
| | | | | | 6600038 | | | |
| NSUN3 | NA | 1086.59 | −11.411576 | uc003drl.1 | chr3: 93845335- | NA | NA | NA |
| | | | | | 93845630 | | | |
| NSUN5P2 | NA | 500.181 | 2.749109 | uc003twp.3 | chr7: 72418837- | NA | NA | NA |
| | | | | | 72419202 | | | |
| NSUN6 | NA | 723.448 | −7.328372 | uc010qcp.1 | chr10: 18834264- | NA | NA | NA |
| | | | | | 18834861 | | | |
| NSUN7 | NA | 237.155 | −4.815741 | uc003gvi.4 | chr4: 40810352- | 540.774 | −6.814837 | uc003gvh.2 |
| | | | | | 40812002 | | | |
| NT5C1B | 610526 | NA | NA | NA | NA | NA | NA | NA |
| NTF3 | 162660 | NA | NA | NA | NA | NA | NA | NA |
| NTMT1 | 613560 | NA | NA | NA | NA | NA | NA | NA |
| NTRK2 | 600456 | NA | NA | NA | NA | NA | NA | NA |
| NTRK3 | 191316 | 460.142 | −6.057215 | uc002bmh.2 | chr15: 88419988- | NA | NA | NA |
| | | | | | 88420165 | | | |
| NUAK1 | 608130 | NA | NA | NA | NA | NA | NA | NA |
| NUB1 | 607981 | 1507.83 | −12.556695 | uc003wjx.3 | chr7: 151074312- | NA | NA | NA |
| | | | | | 151075548 | | | |
| NUBPL | 613621 | NA | NA | NA | NA | 1329.43 | −9.756495 | uc001wrk.4 |
| NUCKS1 | 611912 | NA | NA | NA | NA | 407.823 | −5.118065 | uc001hdb.3 |
| NUDCD1 | 606109 | NA | NA | NA | NA | 1538.01 | −9.378541 | uc003yna.3 |
| NUDCD2 | NA | NA | NA | NA | NA | 939.876 | −9.804752 | uc003lze.3 |
| NUDCD3 | 610296 | NA | NA | NA | NA | NA | NA | NA |
| NUDT10 | 300527 | NA | NA | NA | NA | NA | NA | NA |
| NUDT13 | 609233 | 405.84 | −4.406856 | uc001jtj.3 | chr10: 74890662- | NA | NA | NA |
| | | | | | 74891581 | | | |
| NUDT2 | 602852 | 480.314 | −7.933249 | uc003zub.3 | chr9: 34343439- | NA | NA | NA |
| | | | | | 34343711 | | | |
| NUDT21 | 604978 | NA | NA | NA | NA | 936.603 | −7.900353 | uc002eja.3 |
| NUDT5 | 609230 | NA | NA | NA | NA | NA | NA | NA |
| NUDT7 | 609231 | 314.772 | −8.010681 | uc010chd.3 | chr16: 77775848- | 676.584 | −6.684518 | uc002fff.3 |
| | | | | | 77776157 | | | |
| NUFIP2 | 609356 | NA | NA | NA | NA | 502.702 | −5.447309 | uc002hdx.4 |
| NUMA1 | 164009 | 2134.83 | −12.017527 | uc001orj.2 | chr11: 71713911- | NA | NA | NA |
| | | | | | 71714572 | | | |
| NUMB | 603728 | 758.416 | −3.617743 | uc010ttz.1 | chr14: 73741918- | 548.776 | −6.521037 | uc010ttz.1 |
| | | | | | 73743285 | | | |
| NUP107 | 607617 | NA | NA | NA | NA | 1424.28 | −21.610159 | uc001suf.3 |
| NUP133 | 607613 | 1553.94 | −8.409635 | uc001htn.3 | chr1: 229577044- | NA | NA | NA |
| | | | | | 229577650 | | | |
| NUP160 | 607614 | NA | NA | NA | NA | 952.652 | −5.214878 | uc001ngm.3 |
| NUP188 | 615587 | NA | NA | NA | NA | NA | NA | NA |
| NUP205 | 614352 | 592.149 | −6.040094 | uc003vsw.3 | chr7: 135333305- | NA | NA | NA |
| | | | | | 135333499 | | | |
| NUP210P1 | NA | 1150.42 | −6.745065 | uc003eje.1 | chr3: 126390562- | NA | NA | NA |
| | | | | | 126390782 | | | |
| NUP214 | 114350 | NA | NA | NA | NA | NA | NA | NA |
| NUP35 | 608140 | NA | NA | NA | NA | NA | NA | NA |
| NUP37 | 609264 | 328.138 | −6.492262 | uc001tjc.3 | chr12: 102467973- | NA | NA | NA |
| | | | | | 102468131 | | | |
| NUP62CL | NA | NA | NA | NA | NA | NA | NA | NA |
| NUP85 | 170285 | 714.366 | −6.376368 | uc002jng.1 | chr17: 73231775- | NA | NA | NA |
| | | | | | 73231854 | | | |
| NUP88 | 602552 | 1384.4 | −14.14963 | uc010vsy.2 | chr17: 5291837- | 950.895 | −7.649286 | uc010vsy.2 |
| | | | | | 5292090 | | | |
| NUP93 | 614351 | 2539.09 | −19.565774 | uc002eka.3 | chr16: 56878522- | NA | NA | NA |
| | | | | | 56878861 | | | |
| NUPL1 | NA | NA | NA | NA | NA | 513.757 | −6.855184 | uc001uqg.1 |
| NUSAP1 | 612818 | NA | NA | NA | NA | 754.581 | −7.345449 | uc001znr.4 |
| NVL | 602426 | NA | NA | NA | NA | 464.075 | −6.74485 | uc001hok.3 |
| NXF5 | 300319 | NA | NA | NA | NA | NA | NA | NA |
| NXT2 | 300320 | NA | NA | NA | NA | NA | NA | NA |
| OARD1 | 614393 | 704.066 | −3.023456 | uc003opm.3 | chr6: 41034531- | 341.113 | −5.962321 | uc003opm.3 |
| | | | | | 41035073 | | | |
| OAS1 | 164350 | 696.483 | −7.377879 | uc010syn.2 | chr12: 113354581- | NA | NA | NA |
| | | | | | 113354760 | | | |
| OAS3 | 603351 | NA | NA | NA | NA | 1277.61 | −9.938905 | uc001tue.3 |
| OAT | 613349 | NA | NA | NA | NA | 383.345 | −4.988412 | uc001lhp.3 |
| OBFC1 | 613128 | NA | NA | NA | NA | 349.108 | −6.181194 | uc001kxl.3 |
| OC90 | 601658 | NA | NA | NA | NA | NA | NA | NA |
| OCEL1 | NA | NA | NA | NA | NA | NA | NA | NA |
| ODAM | 614843 | NA | NA | NA | NA | NA | NA | NA |
| OGFR | 606459 | NA | NA | NA | NA | NA | NA | NA |
| OGN | 602383 | 324.345 | −5.641394 | uc004asa.3 | chr9: 95146249- | NA | NA | NA |
| | | | | | 95147901 | | | |
| OGT | 300255 | NA | NA | NA | NA | NA | NA | NA |
| OLFM4 | 614061 | NA | NA | NA | NA | NA | NA | NA |
| OLFML1 | NA | 642.989 | −4.473998 | uc001mfi.3 | chr11: 7531420- | NA | NA | NA |
| | | | | | 7532606 | | | |
| OLFML2B | NA | 1425.58 | −15.758764 | uc001gbt.3 | chr1: 161952982- | NA | NA | NA |
| | | | | | 161953464 | | | |
| OLFML3 | 610088 | NA | NA | NA | NA | 1807.66 | −8.612216 | uc001eer.1 |
| OOEP | 611689 | 292.533 | −8.403957 | uc003pgu.4 | chr6: 74078280- | NA | NA | NA |
| | | | | | 74078506 | | | |
| OOSP2 | NA | NA | NA | NA | NA | 207.785 | −4.690336 | uc001nol.3 |
| OPN4 | 606665 | NA | NA | NA | NA | 1004.95 | −11.790599 | uc009xsx.1 |
| OPRK1 | 165196 | NA | NA | NA | NA | NA | NA | NA |
| OPRM1 | 600018 | NA | NA | NA | NA | 793.474 | −5.345192 | uc011efd.2 |
| OR11A1 | NA | NA | NA | NA | NA | NA | NA | NA |
| OR12D3 | NA | NA | NA | NA | NA | 1114.57 | −10.821066 | uc003nme.3 |
| OR13A1 | NA | NA | NA | NA | NA | 1183.54 | −4.462235 | uc001jcc.1 |
| OR2A9P | NA | NA | NA | NA | NA | NA | NA | NA |
| OR2B3 | NA | 599.185 | −6.516424 | uc003nlx.3 | chr6: 29053985- | NA | NA | NA |
| | | | | | 29054083 | | | |
| OR2H1 | NA | 951.163 | −9.567343 | uc003nmi.3 | chr6: 29430498- | NA | NA | NA |
| | | | | | 29432099 | | | |
| OR2J3 | 615016 | 578.636 | −5.125223 | uc011hhl.2 | chr6_mcf_hap5: | NA | NA | NA |
| | | | | | 444969-445896 | | | |
| OR4C12 | NA | 475.977 | −4.213754 | uc010ria.2 | chr11: 50003009- | NA | NA | NA |
| | | | | | 50003107 | | | |
| OR51B5 | NA | 541.571 | −6.948222 | uc001maq.2 | chr11: 5363744- | NA | NA | NA |
| | | | | | 5363815 | | | |
| OR51D1 | NA | NA | NA | NA | NA | NA | NA | NA |
| OR51E2 | 611268 | NA | NA | NA | NA | NA | NA | NA |
| OR52K1 | NA | NA | NA | NA | NA | 501.996 | −7.847095 | uc001lza.2 |
| OR7E12P | NA | NA | NA | NA | NA | 1035.97 | −7.679921 | uc021qcl.1 |
| OR9Q1 | NA | NA | NA | NA | NA | 790.619 | −9.979769 | uc001nmj.3 |
| ORC1 | 601902 | NA | NA | NA | NA | 590.481 | −5.511061 | uc001ctt.3 |
| ORC4 | 603056 | NA | NA | NA | NA | NA | NA | NA |
| ORC6 | 607213 | 361.918 | −3.792813 | uc010cbe.2 | chr16: 46729622- | 581.62 | −5.39511 | uc002eeg.1 |
| | | | | | 46730131 | | | |
| ORMDL1 | 610073 | 771.925 | −9.284996 | uc002urb.4 | chr2: 190634993- | NA | NA | NA |
| | | | | | 190636492 | | | |
| ORMDL2 | 610074 | NA | NA | NA | NA | NA | NA | NA |
| OSBP2 | 606729 | NA | NA | NA | NA | NA | NA | NA |
| OSBPL10 | 606738 | NA | NA | NA | NA | NA | NA | NA |
| OSBPL1A | 606730 | NA | NA | NA | NA | 505.611 | −6.06863 | uc002kvd.3 |
| OSBPL2 | 606731 | 515.633 | −6.797588 | uc002yck.1 | chr20: 60868944- | NA | NA | NA |
| | | | | | 60871269 | | | |
| OSBPL35 | 606732 | 242.951 | −4.186842 | uc003sxj.1 | chr7: 24873706- | 804.477 | −5.736782 | uc003sxf.3 |
| | | | | | 24873921 | | | |
| OSGEPL1 | NA | NA | NA | NA | NA | 833.451 | −9.161101 | uc002uqz.1 |
| OSMR | 601743 | 610.547 | −5.021246 | uc003jln.2 | chr5: 38933547- | NA | NA | NA |
| | | | | | 38935743 | | | |
| OTOF | 603681 | 414.999 | −4.230366 | uc010yla.2 | chr2: 26680071- | NA | NA | NA |
| | | | | | 26680907 | | | |
| OTOGL | 614925 | NA | NA | NA | NA | NA | NA | NA |
| OTOR | 606067 | NA | NA | NA | NA | 246.678 | −4.316653 | uc002wpj.3 |
| OTUD1 | 612022 | 1004.98 | −9.711742 | uc001irr.2 | chr10: 23729833- | NA | NA | NA |
| | | | | | 23731310 | | | |
| OTUD6B | 612021 | NA | NA | NA | NA | NA | NA | NA |
| OTUD7A | 612024 | NA | NA | NA | NA | NA | NA | NA |
| OTX1 | 600036 | NA | NA | NA | NA | 540.091 | −5.756144 | uc021vim.1 |
| OVOL2 | 616441 | 644.599 | −5.276431 | uc002wqi.1 | chr20: 18004796- | 211.626 | −5.009895 | uc002wqi.1 |
| | | | | | 18005279 | | | |
| OXA1L | 601066 | NA | NA | NA | NA | NA | NA | NA |
| OXCT1 | 601424 | NA | NA | NA | NA | 2221.41 | −20.556358 | uc011cpo.2 |
| OXGR1 | 606922 | NA | NA | NA | NA | NA | NA | NA |
| OXSR1 | 604046 | NA | NA | NA | NA | 955.056 | −8.707562 | uc010hha.1 |
| P2RY1 | 601167 | NA | NA | NA | NA | 406.501 | −6.97294 | uc003ezq.3 |
| P2RY10 | 300529 | NA | NA | NA | NA | NA | NA | NA |
| P2RY12 | 600515 | 459.342 | −6.688892 | uc011boa.2 | chr3: 151055236- | NA | NA | NA |
| | | | | | 151056023 | | | |
| P4HA1 | 176710 | 517.008 | −9.569027 | uc021ptj.1 | chr10: 74766980- | 933.887 | −8.58822 | uc021ptj.1 |
| | | | | | 74767979 | | | |
| P4HA2 | 600608 | NA | NA | NA | NA | 801.578 | −5.149202 | uc003kwg.3 |
| PABPC1L | NA | 219.178 | −4.841167 | uc010ggv.1 | chr20: 43567767- | NA | NA | NA |
| | | | | | 43567962 | | | |
| PABPC4L | NA | NA | NA | NA | NA | NA | NA | NA |
| PABPC5 | 300407 | NA | NA | NA | NA | 835.341 | −5.648522 | uc004efg.3 |
| PACRG | 608427 | 333.476 | −3 420584 | uc003qua.3 | chr6: 163736020- | 263.223 | −7.140932 | uc003qua.3 |
| | | | | | 163736524 | | | |
| PACGRL | NA | 207.18 | −4.351278 | uc003gpz.3 | chr4: 20728965- | 627.538 | −4.454362 | uc003gpz.3 |
| | | | | | 20729980 | | | |
| PACS2 | 610423 | 401.913 | −7.538082 | uc001yqs.2 | chr14: 105861010- | 620.586 | −3.372642 | uc001yqs.2 |
| | | | | | 105864484 | | | |
| PADI4 | 605347 | NA | NA | NA | NA | NA | NA | NA |
| PAFAH2 | 602344 | 594.317 | −6.327632 | uc001bld.4 | chr1«6286258- | NA | NA | NA |
| | | | | | 26288479 | | | |
| PAGE5 | NA | NA | NA | NA | NA | 401.184 | −5.668802 | uc004duj.3 |
| PAH | 612349 | NA | NA | NA | NA | NA | NA | NA |
| PAK1IP1 | 607811 | NA | NA | NA | NA | 1345.98 | −9.758406 | uc003mzg.3 |
| PAK2 | 605022 | 548.153 | −5.264453 | uc003fwy.4 | chr3: 196555277- | NA | NA | NA |
| | | | | | 196559518 | | | |
| PAK6 | 608110 | NA | NA | NA | NA | NA | NA | NA |
| PALM2 | NA | 529.829 | −3.370613 | uc004beg.3 | chr9: 112705706- | 489.416 | −5.532302 | uc004beg.3 |
| | | | | | 112713756 | | | |
| PALMD | 610182 | 1067.21 | −10.364466 | uc001dsg.3 | chr1: 100159619- | NA | NA | NA |
| | | | | | 100160097 | | | |
| PAMR1 | NA | NA | NA | NA | NA | NA | NA | NA |
| PAN2 | NA | NA | NA | NA | NA | 926.009 | −6.691851 | uc001skw.3 |
| PAN3 | NA | NA | NA | NA | NA | 692.323 | −6.248338 | uc010tdo.1 |
| PANK3 | 606161 | 633.955 | −7.588978 | uc003lzz.2 | chr5: 167982628- | NA | NA | NA |
| | | | | | 167984575 | | | |
| PANK4 | 606162 | 867.77 | −8.933503 | uc001ajm.1 | chr1«439975- | NA | NA | NA |
| | | | | | 2440285 | | | |
| PAPD4 | 614121 | NA | NA | NA | NA | NA | NA | NA |
| PAPD5 | 605540 | NA | NA | NA | NA | NA | NA | NA |
| PAPSS2 | 603005 | NA | NA | NA | NA | 450.749 | −5.999988 | uc001kew.3 |
| PAQR9 | 614580 | NA | NA | NA | NA | NA | NA | NA |
| PARD3B | NA | NA | NA | NA | NA | NA | NA | NA |
| PARD6A | 607484 | NA | NA | NA | NA | 815.795 | −5.605366 | uc002ets.3 |
| PARK2 | 602544 | 261.093 | −5.776608 | uc011egf.2 | chr6: 161768590- | NA | NA | NA |
| | | | | | 161771130 | | | |
| PARL1 | 607858 | 533.606 | −10.429358 | uc003fmd.3 | chr3: 183547173- | NA | NA | NA |
| | | | | | 183547385 | | | |
| PARP11 | NA | 643.692 | −5.576862 | uc001qmk.1 | chr12: 3918027- | NA | NA | NA |
| | | | | | 3921288 | | | |
| PARP12 | 612481 | NA | NA | NA | NA | NA | NA | NA |
| PARP14 | 610028 | 1242.51 | −10.457936 | uc003efq.4 | chr3: 122447445- | NA | NA | NA |
| | | | | | 122449687 | | | |
| PARP3 | 607726 | NA | NA | NA | NA | NA | NA | NA |
| PARP4 | 607519 | NA | NA | NA | NA | NA | NA | NA |
| PARP8 | NA | NA | NA | NA | NA | NA | NA | NA |
| PARP9 | 612065 | NA | NA | NA | NA | 397.416 | −5.973242 | uc003eff.4 |
| PARPBP | 613687 | 642.365 | −7.392803 | uc001tjf.3 | chr12: 102590070- | NA | NA | NA |
| | | | | | 102591298 | | | |
| PARS2 | 612036 | NA | NA | NA | NA | NA | NA | NA |
| PASK | 607505 | NA | NA | NA | NA | 458.445 | −7.313594 | uc010zol.2 |
| PATE2 | NA | 494.224 | −5.44058 | uc001qcu.3 | chr11: 125646028- | 492.23 | −5.788252 | uc001qcu.3 |
| | | | | | 125647276 | | | |
| PATE3 | NA | NA | NA | NA | NA | 544.163 | −6.188894 | uc009zbs.3 |
| PAX6 | 607108 | NA | NA | NA | NA | NA | NA | NA |
| PAXBP1 | NA | 611.603 | −5.70694 | uc002yql.3 | chr21: 34106210- | NA | NA | NA |
| | | | | | 34107260 | | | |
| PAXIP1 | 608254 | 1256.66 | −7.280952 | uc003wlr.1 | chr7: 154759030- | NA | NA | NA |
| | | | | | 154759498 | | | |
| PBX4 | 608127 | 401.121 | −5.755988 | uc002nmy.3 | chr19: 19672516- | NA | NA | NA |
| | | | | | 19672833 | | | |
| PBXIP1 | NA | 555.539 | −5.706563 | uc001ffr.3 | chr1: 154916559- | NA | NA | NA |
| | | | | | 154917499 | | | |
| PCCA | 232000 | 292.865 | −6.53282 | uc001voo.3 | chr13: 101182421- | NA | NA | NA |
| | | | | | 101182691 | | | |
| PCDH10 | 608286 | 499.907 | −3.593354 | uc003igz.3 | chr4: 134073987- | 559.693 | −10.263184 | uc003igz.3 |
| | | | | | 134074404 | | | |
| PCDH11X | 300246 | NA | NA | NA | NA | NA | NA | NA |
| PCDH15 | 605514 | 675.172 | −10.476841 | uc021pqy.1 | chr10: 55580860- | 565.855 | −7.883029 | uc021pqy.1 |
| | | | | | 55581617 | | | |
| PCDH17 | 611760 | 377.017 | −4.196924 | uc001vhq.1 | chr13: 58299429- | NA | NA | NA |
| | | | | | 58303065 | | | |
| PCDH19 | 300460 | NA | NA | NA | NA | 1247.65 | −9.008373 | uc004efw.4 |
| PCDH9 | 603581 | NA | NA | NA | NA | 601.513 | −7.674885 | uc001vin.3 |
| PCDHA1 | 606307 | 488.753 | −8.22283 | uc003lgz.3 | chr5: 140168300- | 1061.96 | −16.161703 | NA |
| | | | | | 140168445 | | | |
| PCDHAC1 | 606320 | NA | NA | NA | NA | NA | NA | NA |
| PCDHB10 | 606336 | NA | NA | NA | NA | NA | NA | NA |
| PCDHB11 | 606337 | NA | NA | NA | NA | 1032.31 | −7.242553 | uc003liy.3 |
| PCDHB14 | 606340 | NA | NA | NA | NA | 1213.71 | −11.32538 | uc003ljb.3 |
| PCDHB2 | 606328 | NA | NA | NA | NA | NA | NA | NA |
| PCDHB6 | 606332 | 788.112 | −9.018265 | uc003lir.3 | chr5: 140532224- | 933.566 | −9.613379 | uc003lir.3 |
| | | | | | 140532868 | | | |
| PCDHGA11 | 606298 | NA | NA | NA | NA | 538.09 | −8.24788 | uc003lko.1 |
| PCDHGA7 | 606294 | 297.856 | −8.12457 | uc003ljz.1 | chr5: 140764921- | 1026.19 | −8.486555 | uc003ljz.1 |
| | | | | | 140765187 | | | |
| PCDHGB7 | 606304 | NA | NA | NA | NA | 418.526 | −6.39219 | uc003lkm.3 |
| PCF11 | 608876 | NA | NA | NA | NA | NA | NA | NA |
| PCGF2 | 600346 | NA | NA | NA | NA | 452.248 | −6.464928 | uc002hqp.1 |
| PCIF1 | NA | 872.197 | −9.683287 | uc002xqt.3 | chr20: 44576253- | NA | NA | NA |
| | | | | | 44576662 | | | |
| PCK2 | 614095 | NA | NA | NA | NA | 690.169 | −8.520481 | uc001wls.3 |
| PCLO | 604918 | 1034.99 | −14.206451 | uc003uhx.2 | chr7: 82383321- | NA | NA | NA |
| | | | | | 82387890 | | | |
| PCMT1 | 176851 | 584.573 | −5.996024 | uc003qna.3 | chr6: 150123516- | 637.385 | −6.893598 | uc003qna.3 |
| | | | | | 150123667 | | | |
| PCNX | NA | NA | NA | NA | NA | 624.877 | −8.115316 | uc001xmn.4 |
| PCNXL3 | NA | NA | NA | NA | NA | NA | NA | NA |
| PCNXL4 | NA | NA | NA | NA | NA | 445.943 | −10.562603 | uc001xer.4 |
| PCOLCE | 600270 | NA | NA | NA | NA | NA | NA | NA |
| PCP4L1 | NA | NA | NA | NA | NA | 567.089 | −5.768709 | uc001gad.3 |
| PCSK1 | 162150 | 1048.32 | −10.274687 | uc010jbi.2 | chr5: 95726040- | NA | NA | NA |
| | | | | | 95728704 | | | |
| PCSK5 | 600488 | NA | NA | NA | NA | NA | NA | NA |
| PCYT1B | 300948 | 582.087 | −7.302437 | uc004dbi.3 | chrX: 24576204- | NA | NA | NA |
| | | | | | 24580409 | | | |
| PCYT2S | 602679 | NA | NA | NA | NA | NA | NA | NA |
| PDC | 171490 | NA | NA | NA | NA | 411.01 | −4.987813 | uc001grz.3 |
| PDCD2 | 600866 | 1112.35 | −0.785064 | uc003qxv.3 | chr6: 170884660- | NA | NA | NA |
| | | | | | 170886646 | | | |
| PDCD4 | 608610 | NA | NA | NA | NA | NA | NA | NA |
| PDCD5 | 604583 | 1061.37 | −6.421878 | uc002ntm.3 | chr19: 33078207- | NA | NA | NA |
| | | | | | 33078358 | | | |
| PDE10A | 610652 | 623.543 | −6.382891 | uc011egk.2 | chr6: 165740776- | NA | NA | NA |
| | | | | | 165746513 | | | |
| PDE12 | 616519 | 877.9 | −10.671512 | uc003div.3 | chr3: 57545148- | NA | NA | NA |
| | | | | | 57546664 | | | |
| PDE1C | 602987 | 208.923 | −4.814194 | uc003tcm.2 | chr7: 31792632- | NA | NA | NA |
| | | | | | 31792997 | | | |
| PDE3B | 602047 | 600.178 | −13.980412 | uc001mlm.2 | chr11: 14793588- | NA | NA | NA |
| | | | | | 14798209 | | | |
| PDE4C | 600128 | NA | NA | NA | NA | NA | NA | NA |
| PDE4D | 600129 | 496.29 | −5.276431 | uc003jrs.2 | chr5: 58264866- | NA | NA | NA |
| | | | | | 58270490 | | | |
| PDE5A | 603310 | NA | NA | NA | NA | 1709.54 | −17.043792 | uc003idf.3 |
| PDE6B | 180072 | 859.179 | −4.263594 | uc003gao.4 | chr4: 663897- | NA | NA | NA |
| | | | | | 664681 | | | |
| PDE6D | 602676 | 632.005 | −5.149074 | uc002vse.1 | chr2: 232597147- | NA | NA | NA |
| | | | | | 232597661 | | | |
| PDE7A | 171885 | NA | NA | NA | NA | 281.197 | −8.368252 | uc003xvp.3 |
| PDE8A | 602972 | NA | NA | NA | NA | NA | NA | NA |
| PDGFB | 190040 | 1433.7 | −9.856361 | uc003axe.3 | chr22: 39619685- | NA | NA | NA |
| | | | | | 39621318 | | | |
| PDGFC | 608452 | NA | NA | NA | NA | NA | NA | NA |
| PDGFRA | 173490 | NA | NA | NA | NA | NA | NA | NA |
| PDHB | 179060 | 1144.94 | −6.088681 | uc003dke.4 | chr3: 58413357- | NA | NA | NA |
| | | | | | 58413760 | | | |
| PDHX | 608769 | NA | NA | NA | NA | NA | NA | NA |
| PDIA4 | NA | NA | NA | NA | NA | 780.513 | −5.202882 | uc003wff.2 |
| PDIA6 | 611099 | 313.681 | −4.067054 | uc002rau.3 | chr2: 10923517- | NA | NA | NA |
| | | | | | 10924383 | | | |
| PDIK1L | 610785 | NA | NA | NA | NA | 1051.13 | −8.094871 | uc010oew.2 |
| PDILT | NA | 544.418 | −8.520297 | uc002dhc.1 | chr16: 20370492- | NA | NA | NA |
| | | | | | 20370640 | | | |
| PDK1 | 602524 | NA | NA | NA | NA | 569.163 | −6.980223 | uc002uhs.3 |
| PDLIM4 | 603422 | 297.33 | −5.294382 | uc003kwn.3 | chr5: 131607923- | 231.747 | −3.171367 | uc003kwp.3 |
| | | | | | 131609147 | | | |
| PDLIM7 | 605903 | 584.754 | −4.805341 | uc003mha.1 | chr5: 176910395- | NA | NA | NA |
| | | | | | 176910644 | | | |
| PDPR | NA | 707.093 | −5.11112 | uc002eyf.1 | chr16: 70190783- | NA | NA | NA |
| | | | | | 70195184 | | | |
| PDS5A | 613200 | NA | NA | NA | NA | NA | NA | NA |
| PDS5B | 605333 | 1130.1 | −7.054615 | uc001uun.3 | chr13: 33226202- | NA | NA | NA |
| | | | | | 33226715 | | | |
| PDSS1 | 607429 | 868.32 | −1.264631 | uc001isv.3 | chr10: 27035403- | 734.638 | −4.814194 | uc001isv.3 |
| | | | | | 27035726 | | | |
| PDSS2 | 610564 | 338.293 | −4.355081 | uc011eak.1 | chr6: 107473761- | NA | NA | NA |
| | | | | | 107475822 | | | |
| PDZD2 | 610697 | 788.778 | −11.025304 | uc003jhl.3 | chr5: 32108242- | NA | NA | NA |
| | | | | | 32111038 | | | |
| PDZD9 | NA | 450.98 | −6.119854 | uc002dka.2 | chr16: 21995186- | NA | NA | NA |
| | | | | | 21995587 | | | |
| PDZK1 | 603831 | NA | NA | NA | NA | NA | NA | NA |
| PDZK1IP1 | 607178 | NA | NA | NA | NA | 372.419 | −5.418603 | uc001cqw.3 |
| PDZRN3 | 609729 | NA | NA | NA | NA | 402.155 | −6.323867 | uc011bgf.1 |
| PDZRN4 | 609730 | NA | NA | NA | NA | 522.218 | −8.162259 | uc010skn.2 |
| PEAK1 | 614248 | 829.457 | −4.534261 | uc002bcn.2 | chr15: 77447542- | NA | NA | NA |
| | | | | | 77450812 | | | |
| PEAR1 | 610278 | NA | NA | NA | NA | 311.496 | −4.008825 | uc001fqj.1 |
| PEBP1 | 604591 | NA | NA | NA | NA | 715.174 | −4.682318 | uc010szc.1 |
| PEG10 | 609810 | NA | NA | NA | NA | NA | NA | NA |
| PER2 | 603426 | 634.171 | −0.951344 | uc002vyc.3 | chr2: 239152679- | NA | NA | NA |
| | | | | | 239155015 | | | |
| PET117 | 614771 | 1712.38 | −16.748124 | uc021wba.1 | chr20: 18123002- | NA | NA | NA |
| | | | | | 18123812 | | | |
| PEX1 | 602136 | 748.097 | −8.969383 | uc003uly.3 | chr7: 92116337- | NA | NA | NA |
| | | | | | 92116770 | | | |
| PEX13 | 601789 | 793.139 | −7.143347 | uc002sau.4 | chr2: 61275906- | 746.19 | −6.329673 | uc002sau.4 |
| | | | | | 61279125 | | | |
| PEX3 | 603164 | 1201.46 | −1.258677 | uc011edx.1 | chr6: 143793481- | NA | NA | NA |
| | | | | | 143793620 | | | |
| PEX7 | 601757 | NA | NA | NA | NA | NA | NA | NA |
| PFDN2 | 613466 | NA | NA | NA | NA | 387.502 | −5.01218 | uc001fxu.3 |
| PFDN4 | 604898 | 1021.35 | −12.702964 | uc002xwx.3 | chr20: 52835690- | NA | NA | NA |
| | | | | | 52836492 | | | |
| PFKM | 610681 | NA | NA | NA | NA | NA | NA | NA |
| PFN2 | 176590 | NA | NA | NA | NA | 778.72 | −4.492359 | uc003exu.1 |
| PGAP1 | 611655 | NA | NA | NA | NA | NA | NA | NA |
| PGBD1 | NA | NA | NA | NA | NA | 231.344 | −12.74781 | uc003nky.3 |
| PGD | 172200 | 627.522 | −5.370967 | uc001arc.3 | chr1: 10479807- | NA | NA | NA |
| | | | | | 10480201 | | | |
| PGK1 | 311800 | NA | NA | NA | NA | 384.66 | −5.31166 | uc004ecz.4 |
| PGM3 | 172100 | 574.557 | −9.793614 | uc003pjw.3 | chr6: 83874593- | 1544.97 | −2.988636 | uc003pju.2 |
| | | | | | 83879016 | | | |
| PGR | 607311 | 973.049 | −13.015832 | uc001pgg.2 | chr11: 100900355- | NA | NA | NA |
| | | | | | 100909846 | | | |
| PGRMC1 | 300435 | 620.246 | −6.630253 | uc004erb.3 | chrX: 118377218- | 567.642 | −9.808776 | uc004erb.3 |
| | | | | | 118378429 | | | |
| PHACTR1 | 608723 | 675.953 | −4.597364 | uc003nag.2 | chr6: 13273156- | 729.814 | −6.279615 | uc003nah.2 |
| | | | | | 13276355 | | | |
| PHACTR4 | 608726 | 1554.13 | −6.132852 | uc001bpu.3 | chr1: 28793288- | NA | NA | NA |
| | | | | | 28793725 | | | |
| PHB | 176705 | 442.829 | −5.625484 | uc002iox.1 | chr17: 47481420- | NA | NA | NA |
| | | | | | 47482353 | | | |
| PHF1 | 602881 | NA | NA | NA | NA | 521.631 | −6.641573 | uc003oei.3 |
| PHF10 | 613069 | NA | NA | NA | NA | 878.355 | −7.522051 | uc011eha.1 |
| PHF12 | NA | 363.255 | −4.821629 | uc010wbb.1 | chr17: 27232271- | NA | NA | NA |
| | | | | | 27233200 | | | |
| PHF13 | NA | 697.758 | −5.552244 | uc001aob.4 | chr1: 6681698- | NA | NA | NA |
| | | | | | 6684093 | | | |
| PHF14 | NA | 1153.44 | −6.791433 | uc003sry.2 | chr7: 11142126- | 806.405 | −5.877645 | uc003sry.2 |
| | | | | | 11147376 | | | |
| PHF2 | 604351 | NA | NA | NA | NA | 1505.3 | −25.528641 | uc004aub.3 |
| PHF21A | 608325 | 970.016 | −11.868692 | uc001ncb.4 | chr11: 45950870- | NA | NA | NA |
| | | | | | 45955518 | | | |
| PHF6 | 300414 | NA | NA | NA | NA | 794.356 | −5.493047 | uc004exh.3 |
| PHF8 | 300560 | 379.28 | −5.885366 | uc004dst.3 | chrX: 53963113- | 222.825 | −9.968539 | uc004dsx.3 |
| | | | | | 53965590 | | | |
| PHKG2 | 172471 | NA | NA | NA | NA | NA | NA | NA |
| PHLDA1 | 605335 | NA | NA | NA | NA | NA | NA | NA |
| PHLDB1 | 612834 | NA | NA | NA | NA | 203.858 | −4.587647 | uc001ptr.2 |
| PHLDB3 | NA | 333.675 | −4.36515 | uc010eit.3 | chr19: 43979255- | 378.916 | −6.167544 | uc010eit.3 |
| | | | | | 43979561 | | | |
| PHLPP2 | 611066 | NA | NA | NA | NA | NA | NA | NA |
| PHOX2B | 603851 | NA | NA | NA | NA | NA | NA | NA |
| PHTF1 | 604950 | 868.403 | −11.733697 | uc001edo.1 | chr1: 114248389- | NA | NA | NA |
| | | | | | 114248454 | | | |
| PHYH | 602026 | NA | NA | NA | NA | 202.417 | −4.150326 | uc001ime.3 |
| PHYHIPL | NA | NA | NA | NA | NA | NA | NA | NA |
| PHYKPL | 614683 | NA | NA | NA | NA | 502.737 | −5.005975 | uc003mjd.1 |
| PI15 | 607076 | NA | NA | NA | NA | 1372.93 | −10.981316 | uc003yal.3 |
| PI4K2B | 612101 | NA | NA | NA | NA | 243.415 | −8.194586 | uc011bxs.2 |
| PI4KA | 600286 | NA | NA | NA | NA | NA | NA | NA |
| PIAS2 | 603567 | 439.522 | −6.338471 | uc002lck.3 | chr18: 44390023- | 560.806 | −7.049207 | uc002lcl.3 |
| | | | | | 44392395 | | | |
| PIAS4 | 605989 | NA | NA | NA | NA | NA | NA | NA |
| PIBF1 | 607532 | 319.976 | −5.68271 | uc001vjc.3 | chr13: 73590058- | NA | NA | NA |
| | | | | | 73590591 | | | |
| PIEZO2 | 613629 | 469.376 | −6.993796 | uc002koq.3 | chr18: 10670244- | 852.17 | −6.216005 | uc002koq.3 |
| | | | | | 10671523 | | | |
| PIFO | 614234 | 731.029 | −3.653042 | uc001eaw.2 | chr1: 111893950- | NA | NA | NA |
| | | | | | 111895639 | | | |
| PIGA | 311770 | NA | NA | NA | NA | NA | NA | NA |
| PIGF | 600153 | 1159.4 | −8.571925 | uc002rvc.3 | chr2: 46808413- | 1249.99 | −5.512043 | uc002rvc.3 |
| | | | | | 46808730 | | | |
| PIGG | NA | 1067.56 | −8.422388 | uc011buw.2 | chr4: 515555- | 296.732 | −5.069151 | uc011buw.2 |
| | | | | | 516632 | | | |
| PIGK | 605087 | NA | NA | NA | NA | NA | NA | NA |
| PIGN | 606097 | NA | NA | NA | NA | 900.707 | −10.069057 | uc021ulb.1 |
| PIGP | 605938 | NA | NA | NA | NA | 661.024 | −8.407417 | uc002yvw.1 |
| PIGQ | 605754 | NA | NA | NA | NA | 292.163 | 2.111161 | uc010bqw.3 |
| PIGU | 608528 | 856.094 | −6.207949 | uc010zul.2 | chr20: 33148346- | NA | NA | NA |
| | | | | | 33148783 | | | |
| PIGW | 610275 | 938.791 | −7.644802 | uc002hmy.1 | chr17: 34894466- | NA | NA | NA |
| | | | | | 34895150 | | | |
| PIH1D1 | 611480 | 409.429 | −5.101542 | uc002pns.2 | chr19: 49949550- | 1131.82 | −9.299662 | uc010yaq.2 |
| | | | | | 49949638 | | | |
| PIH1D2 | NA | 366.931 | −9.810662 | uc009yyl.3 | chr11: 111934734- | 618.265 | −2.749135 | uc009yyl.3 |
| | | | | | 111934891 | | | |
| PIK3AP1 | 607942 | 650.098 | −4.935659 | uc001kmo.3 | chr10: 98353069- | NA | NA | NA |
| | | | | | 98355333 | | | |
| PIK3C2A | 603601 | 969.808 | −3.62142 | uc009ygu.1 | chr11: 17099277- | 1235.19 | −6.572311 | uc009ygu.1 |
| | | | | | 17099412 | | | |
| PIK3CA | 171834 | NA | NA | NA | NA | 269.408 | −4.710082 | uc003fjk.3 |
| PIK3CD | 602839 | NA | NA | NA | NA | NA | NA | NA |
| PIK3R3 | 606076 | 833.106 | −6.315417 | uc010olv.2 | chr1: 46505812- | 1066.56 | −9.087541 | uc010olv.2 |
| | | | | | 46509344 | | | |
| PIK3R4 | 602610 | 797.961 | −8.443188 | uc003enj.3 | chr3: 130397778- | NA | NA | NA |
| | | | | | 130398158 | | | |
| PILRA | 605341 | 435.64 | −4.2571 | uc011kjo.2 | chr7: 99997557- | NA | NA | NA |
| | | | | | 99997716 | | | |
| PIM2 | 300295 | 777.631 | −8.529398 | uc004dls.3 | chrX: 48770459- | 376.064 | −4.641796 | uc004dls.3 |
| | | | | | 48771407 | | | |
| PIN4 | 300252 | NA | NA | NA | NA | NA | NA | NA |
| PINK1 | 608309 | NA | NA | NA | NA | NA | NA | NA |
| PIP | 176720 | NA | NA | NA | NA | NA | NA | NA |
| PIP4K2A | 603140 | NA | NA | NA | NA | NA | NA | NA |
| PIP5K1A | 603275 | 559.854 | −8.918435 | uc010pcv.2 | chr1: 151220424- | 720.654 | −6.125561 | uc001exi.3 |
| | | | | | 151222007 | | | |
| PIP5K1P1 | NA | NA | NA | NA | NA | 231.277 | −4.443856 | uc003mxx.4 |
| PIWIL4 | 610315 | 496.911 | −6.697477 | uc001pfa.3 | chr11: 94354159- | NA | NA | NA |
| | | | | | 94354587 | | | |
| PKD1P1 | NA | 266.987 | −2.93942 | NA | NA | 669.682 | −6.941304 | uc002dew.4 |
| PKD2 | 173910 | NA | NA | NA | NA | 823.115 | −9.284738 | uc003hre.3 |
| PKDCC | 614150 | NA | NA | NA | NA | 559.335 | −4.363961 | uc002rsg.3 |
| PKHD1 | 606702 | NA | NA | NA | NA | NA | NA | NA |
| PKIB | 606914 | NA | NA | NA | NA | NA | NA | NA |
| PKNOX1 | 602100 | NA | NA | NA | NA | NA | NA | NA |
| PKP1 | 601975 | NA | NA | NA | NA | NA | NA | NA |
| PKP2 | 602861 | NA | NA | NA | NA | 777.074 | −6.974659 | uc001rlj.4 |
| PKP4 | 604276 | 545.072 | −4.261875 | uc002tzv.3 | chr2: 159537190- | 350.876 | −1.060779 | uc002tzv.3 |
| | | | | | 159537940 | | | |
| PLA1A | 607460 | 462.624 | −4.539498 | uc003ecu.3 | chr3: 119348316- | 506.903 | −6.426749 | uc003ecu.3 |
| | | | | | 119348658 | | | |
| PLA2G16 | 613867 | 356.074 | −6.144294 | uc001nxh.2 | chr11: 63341944- | NA | NA | NA |
| | | | | | 63342416 | | | |
| PLA2G4A | 600522 | NA | NA | NA | NA | NA | NA | NA |
| PLA2G4D | 612864 | 598.955 | −5.076075 | uc001zox.3 | chr15: 42359881- | NA | NA | NA |
| | | | | | 42360912 | | | |
| PLA2G4E | NA | NA | NA | NA | NA | NA | NA | NA |
| PLA2G5 | 601192 | 637.733 | −6.002068 | uc001bcx.3 | chr1: 20417186- | NA | NA | NA |
| | | | | | 20418394 | | | |
| PLA2G7 | 601690 | 1268.93 | −8.76556 | uc011dwd.1 | chr6: 46677401- | 812.188 | −15.84813 | uc010jzf.3 |
| | | | | | 46678263 | | | |
| PLAC1 | 300296 | NA | NA | NA | NA | 1463.1 | −6.237881 | uc004exo.1 |
| PLAC8 | 607515 | 338.968 | −7.637076 | uc003hoe.3 | chr4: 84011211- | NA | NA | NA |
| | | | | | 84012124 | | | |
| PLAC8L1 | NA | NA | NA | NA | NA | 263.037 | −4.120206 | uc003lnv.3 |
| PLAU | 191840 | 884.113 | −10.776448 | uc001jwa.3 | chr10: 75676324- | 224.44 | −2.799258 | uc001jwa.3 |
| | | | | | 75677258 | | | |
| PLB1 | 610179 | NA | NA | NA | NA | 1749.27 | −20.956607 | uc002rmc.3 |
| PLCB2 | 604114 | NA | NA | NA | NA | 210.782 | −5.654323 | uc001zle.4 |
| PLCB3 | 600230 | NA | NA | NA | NA | 465.957 | −4.539498 | uc009ypg.2 |
| PLCB4 | 600810 | 478.844 | −1.6349 | uc010gbx.3 | chr20: 9459637- | 477.292 | −5.871015 | uc010gbx.3 |
| | | | | | 9461462 | | | |
| PLCL1 | 600597 | NA | NA | NA | NA | 891.395 | −10.57252 | uc002uuv.4 |
| PLD1 | 602382 | 1001.1 | −7.575397 | uc003fhs.3 | chr3: 171318195- | NA | NA | NA |
| | | | | | 171320867 | | | |
| PLD5 | NA | 1187.88 | −6.914249 | uc001hzl.4 | chr1: 242251689- | 652.426 | −5.731036 | uc001hzl.4 |
| | | | | | 242253155 | | | |
| PLD6 | 614960 | 309.365 | −4.344823 | uc002gqz.3 | chr17: 17104309- | NA | NA | NA |
| | | | | | 17106080 | | | |
| PLEKHA5 | 607770 | 314.53 | −5.749776 | uc001rdz.4 | chr12: 19353263- | NA | NA | NA |
| | | | | | 19354937 | | | |
| PLEKHB1 | 607651 | NA | NA | NA | NA | NA | NA | NA |
| PLEKHD1 | NA | NA | NA | NA | NA | NA | NA | NA |
| PLEKHG1 | NA | NA | NA | NA | NA | 511.944 | −8.977533 | uc003qny.1 |
| PLEKHG6 | 611743 | NA | NA | NA | NA | 528.819 | −7.606735 | uc001qnr.3 |
| PLEKHH1 | NA | 633.374 | −6.929407 | uc001xjl.1 | chr14: 68053953- | 270.452 | −5.473154 | uc010tex.1 |
| | | | | | 68056255 | | | |
| PLEKHH2 | 612723 | 286.014 | −4.410744 | uc002rtf.3 | chr2: 43968199- | 873.878 | −6.014774 | uc002rte.3 |
| | | | | | 43968500 | | | |
| PLEKHJ1 | NA | NA | NA | NA | NA | 1228.79 | −12.934199 | uc002lvf.1 |
| PLEKHM2 | 609613 | 594.545 | −6.608819 | uc010obo.2 | chr1: 16060430- | NA | NA | NA |
| | | | | | 16061264 | | | |
| PLEKHO1 | 608335 | NA | NA | NA | NA | 951.321 | −6.257365 | uc001ets.3 |
| PLEKHS1 | NA | NA | NA | NA | NA | 362.35 | −5.399189 | uc001lar.2 |
| PLG | 173350 | NA | NA | NA | NA | 665.088 | −11.790675 | uc021zhr.1 |
| PLIN5 | 613248 | NA | NA | NA | NA | NA | NA | NA |
| PLK2 | 607023 | 947.906 | −9.584497 | uc003jrn.3 | chr5: 57749810- | NA | NA | NA |
| | | | | | 57750409 | | | |
| PLK4 | 605031 | NA | NA | NA | NA | NA | NA | NA |
| PLOD2 | 601865 | NA | NA | NA | NA | NA | NA | NA |
| PLRG1 | 605961 | NA | NA | NA | NA | 1068.06 | −8.572879 | uc003iny.3 |
| PLSCR1 | 604170 | NA | NA | NA | NA | NA | NA | NA |
| PLSCR4 | 607612 | 1078.32 | −5.143917 | uc010huy.3 | chr3: 145910124- | NA | NA | NA |
| | | | | | 145912197 | | | |
| PLSCR5 | NA | 1170.69 | −9.845634 | uc010hvb.3 | chr3: 146294395- | NA | NA | NA |
| | | | | | 146294512 | | | |
| PMAIP1 | 604959 | NA | NA | NA | NA | 893.608 | −7.57558 | uc002lic.2 |
| PMCH | 176795 | 517.535 | −8.703149 | uc001tjl.3 | chr12: 102590237- | NA | NA | NA |
| | | | | | 102590429 | | | |
| PMPCA | 613036 | NA | NA | NA | NA | NA | NA | NA |
| PMPCB | 603131 | 1022.97 | −9.390863 | uc010liu.1 | chr7: 102949573- | 510.651 | −6.838519 | uc011kll.1 |
| | | | | | 102949706 | | | |
| PMS2CL | NA | NA | NA | NA | NA | NA | NA | NA |
| PMS2P5 | NA | NA | NA | NA | NA | 452.938 | −9.376961 | uc003ubl.2 |
| PNCK | 300680 | NA | NA | NA | NA | NA | NA | NA |
| PNISR | 616653 | 208.308 | −4.863108 | uc003pps.2 | chr6: 99858451- | 315.484 | −4.630178 | uc003ppo.4 |
| | | | | | 99860189 | | | |
| PNP | 164050 | NA | NA | NA | NA | 206.733 | −3.512989 | uc010ahn.3 |
| POC1B | 614784 | NA | NA | NA | NA | NA | NA | NA |
| POF1B | 300603 | NA | NA | NA | NA | 1665.67 | −7.220709 | uc004eer.2 |
| POFUT1 | 607491 | 600.526 | −10.651106 | uc002wxo.3 | chr20: 30804854- | NA | NA | NA |
| | | | | | 30805726 | | | |
| POFUT2 | 610249 | 446.2 | −5.93492 | uc002zhd.3 | chr21: 46683843- | NA | NA | NA |
| | | | | | 46687365 | | | |
| POGLUT1 | 615618 | NA | NA | NA | NA | 656.802 | −7.707613 | uc003ecm.3 |
| POGZ | 614787 | 473.186 | −7.513237 | uc001eyg.2 | chr1: 151398679- | NA | NA | NA |
| | | | | | 151400284 | | | |
| POLA2 | NA | 398.914 | −5.819525 | uc009yqf.1 | chr11: 65047131- | NA | NA | NA |
| | | | | | 65047491 | | | |
| POLB | 174760 | 468.763 | −9.583439 | uc003xoz.2 | chr8: 42229176- | 914.222 | −8.420926 | uc003xoz.2 |
| | | | | | 42229331 | | | |
| POLDIP2 | 611519 | NA | NA | NA | NA | 824.555 | −8.908061 | uc002haz.3 |
| POLG | 174763 | 502.139 | −5.770486 | uc002bnr.4 | chr15: 89859536- | NA | NA | NA |
| | | | | | 89859981 | | | |
| POLG2 | 604983 | 866.449 | −8.484058 | uc010deg.2 | chr17: 62481711- | 1087.11 | −12.984047 | uc010deg.2 |
| | | | | | 62481820 | | | |
| POLH | 603968 | NA | NA | NA | NA | NA | NA | NA |
| POLI | 605252 | 642.02 | −6.274411 | uc002lfj.4 | chr18: 51820838- | NA | NA | NA |
| | | | | | 51824604 | | | |
| POLR1B | 602000 | NA | NA | NA | NA | 447.076 | −8.419309 | uc002thw.2 |
| POLR1E | NA | NA | NA | NA | NA | NA | NA | NA |
| POLR2B | 180661 | NA | NA | NA | NA | 1876.39 | −17.898689 | uc003hcl.1 |
| POLR2F | 604414 | 221.768 | −5.622688 | uc003aul.3 | chr22: 38363723- | NA | NA | NA |
| | | | | | 38365319 | | | |
| POLR2G | 602013 | 1297.06 | −9.726277 | uc001nva.3 | chr11: 62533980- | NA | NA | NA |
| | | | | | 62534187 | | | |
| POLR2J2 | 609881 | 1043.01 | −8.031869 | uc010lij.2 | chr7: 102277195- | NA | NA | NA |
| | | | | | 102277971 | | | |
| POLR2K | 606033 | 659.743 | −6.52391 | uc003yjf.3 | chr8: 101165545- | 510.104 | −7.626874 | uc003yjf.3 |
| | | | | | 101166230 | | | |
| POLR3GL | NA | NA | NA | NA | NA | 1062.23 | −15.078463 | uc001enp.1 |
| POM121 | 615753 | 1160.55 | −3.937003 | uc010lam.1 | chr7: 72419010- | 822.058 | −5.326524 | uc010lam.1 |
| | | | | | 72421979 | | | |
| POMP | 613386 | NA | NA | NA | NA | 795.082 | −13.288406 | uc001usf.3 |
| PON3 | 602720 | 970.789 | −9.060639 | uc003unt.3 | chr7: 94989184- | 431.955 | −5.933048 | uc011kii.2 |
| | | | | | 94989284 | | | |
| POP5 | 609992 | NA | NA | NA | NA | 317.267 | −5.922878 | uc001tys.3 |
| PORCN | 300651 | NA | NA | NA | NA | 501.795 | −6.062114 | uc004djr.1 |
| POU1F1 | 173110 | NA | NA | NA | NA | NA | NA | NA |
| POU3F2 | 600494 | NA | NA | NA | NA | 296.308 | −5.4862 | uc003ppe.3 |
| POU4F2 | 113725 | NA | NA | NA | NA | 701.264 | −4.20991 | uc003ikv.3 |
| POU6F2 | 609062 | 446.248 | −4.277697 | uc010kxo.3 | chr7: 39413106- | NA | NA | NA |
| | | | | | 39413307 | | | |
| PPA1 | 179030 | NA | NA | NA | NA | 1336.87 | −9.755068 | uc001jqv.1 |
| PPA2 | 609988 | NA | NA | NA | NA | NA | NA | NA |
| PPAP2B | NA | NA | NA | NA | NA | NA | NA | NA |
| PPAPDC1B | NA | NA | NA | NA | NA | 656.258 | −5.469545 | uc003xlg.4 |
| PPARG | 601487 | 693.626 | −7.33651 | uc003bwr.3 | chr3: 12475645- | NA | NA | NA |
| | | | | | 12475855 | | | |
| PPCDC | 609854 | NA | NA | NA | NA | 278.639 | −4.127464 | uc002azo.3 |
| PPEF2 | 602256 | 523.669 | −5.490739 | uc003hix.3 | chr4: 76781026- | 398.708 | −7.627851 | uc003hix.3 |
| | | | | | 76781819 | | | |
| PPFIA2 | 603143 | NA | NA | NA | NA | 843.424 | −9.319295 | uc010sue.2 |
| PPFIBP1 | 603141 | NA | NA | NA | NA | 716.285 | −7.508159 | uc001rhz.2 |
| PPIAL4E | 608608 | NA | NA | NA | NA | 544.896 | −4.739693 | uc010paz.2 |
| PPIAP30 | NA | NA | NA | NA | NA | 487.757 | −4.60718 | uc010qca.1 |
| PPIB | 123841 | NA | NA | NA | NA | NA | NA | NA |
| PPIC | 123842 | NA | NA | NA | NA | 1681.43 | −3.615525 | uc003kth.3 |
| PPID | 601753 | 873.291 | −7.854077 | uc003iqc.3 | chr4: 159630279- | NA | NA | NA |
| | | | | | 159630887 | | | |
| PPIG | 606093 | NA | NA | NA | NA | NA | NA | NA |
| PPIH | 606095 | NA | NA | NA | NA | NA | NA | NA |
| PPM1A | 606108 | NA | NA | NA | NA | NA | NA | NA |
| PPM1B | 603770 | NA | NA | NA | NA | 296.507 | −8.733805 | uc002rtu.3 |
| PPM1N | NA | NA | NA | NA | NA | NA | NA | NA |
| PPP1CB | 600590 | NA | NA | NA | NA | 470.445 | −6.047224 | uc010ymj.2 |
| PPPICC | 176914 | 998.924 | −7.830697 | uc001tru.3 | chr12: 111157613- | 485.983 | −4.588473 | uc001tru.3 |
| | | | | | 111158880 | | | |
| PPP1R11 | 606670 | NA | NA | NA | NA | NA | NA | NA |
| PPP1R12C | 613245 | NA | NA | NA | NA | NA | NA | NA |
| PPP1R14A | 608153 | 839.492 | −7.499396 | uc002ohq.3 | chr19: 38741877- | NA | NA | NA |
| | | | | | 38741957 | | | |
| PPP1R14C | 613242 | NA | NA | NA | NA | NA | NA | NA |
| PPP1R15A | 611048 | NA | NA | NA | NA | 1103.73 | −7.539785 | uc002pky.4 |
| PPP1R15B | 613257 | NA | NA | NA | NA | NA | NA | NA |
| PPP1R1A | 613246 | NA | NA | NA | NA | NA | NA | NA |
| PPP1R1B | 604399 | NA | NA | NA | NA | NA | NA | NA |
| PPP1R2 | 601792 | 633.978 | −16.676052 | uc003fup.3 | chr3: 195241221- | NA | NA | NA |
| | | | | | 195243693 | | | |
| PPP1R26 | 614056 | 227.484 | −6.066031 | uc004cfr.1 | chr9: 138379987- | 232.984 | −4.447864 | uc004cfr.1 |
| | | | | | 138380739 | | | |
| PPP1R2P3 | NA | 821.29 | −10.310598 | uc003lwf.1 | chr5: 156278192- | NA | NA | NA |
| | | | | | 156279539 | | | |
| PPP1R2P9 | NA | NA | NA | NA | NA | NA | NA | NA |
| PPP1R35 | NA | 775.722 | −9.059802 | uc003uuy.1 | chr7: 100032912- | NA | NA | NA |
| | | | | | 100032982 | | | |
| PPP1R3D | 603326 | NA | NA | NA | NA | 1256.95 | −8.296879 | uc002ybb.3 |
| PPP1R7 | 602877 | NA | NA | NA | NA | NA | NA | NA |
| PPP2CA | 176915 | NA | NA | NA | NA | 401.543 | −6.750479 | uc003kze.3 |
| PPP2CB | 176916 | NA | NA | NA | NA | NA | NA | NA |
| PPP2R1B | 603113 | NA | NA | NA | NA | NA | NA | NA |
| PPP2R2D | 613992 | 288.886 | −6.142211 | uc001lkr.3 | chr10: 133769468- | NA | NA | NA |
| | | | | | 133770053 | | | |
| PPP2R3C | 615902 | NA | NA | NA | NA | NA | NA | NA |
| PPP2R4 | 600756 | NA | NA | NA | NA | NA | NA | NA |
| PPP2R5A | 601643 | 460.842 | −7.965048 | uc001hjb.3 | chr1: 212534113- | 333.884 | −6.153444 | uc001hjb.3 |
| | | | | | 212535205 | | | |
| PPP3CA | 114105 | 997.15 | −10.681226 | uc003hvs.2 | chr4: 101944587- | 1193.95 | −10.619017 | uc003hvs.2 |
| | | | | | 101947021 | | | |
| PPP3CB | 114106 | 827.771 | −5.965954 | uc010qkj.2 | chr10: 75196563- | NA | NA | NA |
| | | | | | 75197999 | | | |
| PPP3CC | 114107 | NA | NA | NA | NA | 1243.78 | −7.360191 | uc003xbs.3 |
| PPP3R1 | 601302 | 202.632 | −4.082962 | uc002sei.1 | chr2: 68405989- | 402.076 | −4.244388 | uc002sei.1 |
| | | | | | 68408094 | | | |
| PPP4R2 | 613822 | NA | NA | NA | NA | 740.175 | −11.155499 | uc003dph.1 |
| PPP6R2 | 610877 | NA | NA | NA | NA | 826.329 | −2.349148 | uc003bky.2 |
| PPRC1 | NA | NA | NA | NA | NA | 609.519 | −6.03315 | uc001kum.3 |
| PQBP1 | 300463 | NA | NA | NA | NA | 552.836 | −5.948957 | uc004dlj.1 |
| PQLC1 | NA | NA | NA | NA | NA | NA | NA | NA |
| PQLC2L | NA | 1121.83 | −9.894497 | uc003fbo.3 | chr3: 157296123- | 233.897 | −4.070743 | uc003fbo.3 |
| | | | | | 157296680 | | | |
| PRADC1 | NA | 678.679 | −5.7815 | uc002siy.3 | chr2: 73455134- | NA | NA | NA |
| | | | | | 73455581 | | | |
| PRAMEF1 | NA | 1387.94 | −13.241679 | uc001auj.2 | chr1: 12856146- | 213.607 | −2.502641 | uc001auj.2 |
| | | | | | 12856777 | | | |
| PRAMEF12 | NA | 616.587 | −7.280617 | uc001aui.3 | chr1: 12837743- | NA | NA | NA |
| | | | | | 12838048 | | | |
| PRAMEF20 | NA | 256.344 | −4.418053 | uc009vnv.1 | chr1: 13526874- | NA | NA | NA |
| | | | | | 13526943 | | | |
| PRAMEF5 | NA | NA | NA | NA | NA | 329.938 | −5.898029 | uc001auu.1 |
| PRAMEF8 | NA | 766.477 | −5.070669 | uc001aup.3 | chr1: 12980234- | NA | NA | NA |
| | | | | | 12980568 | | | |
| PRAMEF9 | NA | NA | NA | NA | NA | 342.999 | −4.224731 | uc001auw.1 |
| PRCP | 176785 | 1082.54 | −9.633736 | uc001ozr.3 | chr11: 82535409- | 1428.18 | −13.449655 | uc001ozr.3 |
| | | | | | 82535947 | | | |
| PRDM1 | 603423 | 521.307 | −5.212678 | uc003prd.2 | chr6: 106555362- | 418.965 | −7.283789 | uc003prd.2 |
| | | | | | 106557814 | | | |
| PRDM10 | NA | NA | NA | NA | NA | NA | NA | NA |
| PRDM13 | NA | 548.061 | −5.52181 | uc003pqg.1 | chr6: 100062636- | NA | NA | NA |
| | | | | | 100063454 | | | |
| PRDM15 | NA | NA | NA | NA | NA | NA | NA | NA |
| PRDX4 | 300927 | 547.556 | −5.170307 | uc004dam.3 | chrX: 23704453- | NA | NA | NA |
| | | | | | 23704514 | | | |
| PRELID2 | NA | 934.445 | −11.875292 | uc003lno.1 | chr5: 145138582- | NA | NA | NA |
| | | | | | 145140088 | | | |
| PREPL | 609557 | NA | NA | NA | NA | NA | NA | NA |
| PRIM2 | 176636 | 927.196 | −5.490174 | uc003pdv.1 | chr6: 57190862- | 658.205 | −7.02892 | uc003pdx.3 |
| | | | | | 57191161 | | | |
| PRIMA1 | 613851 | NA | NA | NA | NA | 387.932 | −5.101985 | uc001ybw.1 |
| PRIMPOL | 615421 | 227.145 | −7.481932 | uc003iwj.2 | chr4: 185615934- | NA | NA | NA |
| | | | | | 185616112 | | | |
| PRKAA1 | 602739 | 444.15 | −3.123422 | uc003jmb.3 | chr5: 40759481- | 851.324 | −6.572413 | uc003jmb.3 |
| | | | | | 40762879 | | | |
| PRKAA2 | 600497 | 1052.34 | −5.304457 | uc001cyk.4 | chr1: 57173387- | 231.326 | −7.040073 | uc001cyk.4 |
| | | | | | 57181008 | | | |
| PRKAB2 | 602741 | 351.258 | −4.151407 | uc001epe.3 | chr1: 146626685- | 1375.11 | −8.72506 | uc009wjf.1 |
| | | | | | 146631143 | | | |
| PRKACA | 601639 | 1039.16 | −8.187108 | uc002myb.3 | chr19: 14202507- | 1067.45 | −1.769593 | uc002myb.3 |
| | | | | | 14203923 | | | |
| PRKACB | 176892 | NA | NA | NA | NA | NA | NA | NA |
| PRKACG | 176893 | 1082.75 | −9.850744 | uc004agy.3 | chr9: 71627449- | 600.584 | −6.219539 | uc004agy.3 |
| | | | | | 71627952 | | | |
| PRKAR1B | 176911 | NA | NA | NA | NA | 208.692 | −5.59229 | uc003siu.2 |
| PRKCB | 176970 | NA | NA | NA | NA | NA | NA | NA |
| PRKCZ | 176982 | NA | NA | NA | NA | NA | NA | NA |
| PRKD3 | 607077 | 1227.42 | −8.435821 | uc002rqf.1 | chr2: 37494711- | NA | NA | NA |
| | | | | | 37496698 | | | |
| PRKDC | 600899 | NA | NA | NA | NA | 692.004 | −6.02719 | uc003xqi.3 |
| PRKG1 | 176894 | 1270.96 | −7.124208 | uc001jjm.3 | chr10: 54053661- | NA | NA | NA |
| | | | | | 54058110 | | | |
| PRKRA | 603424 | 654.975 | −6.420746 | uc002umc.3 | chr2: 179296141- | NA | NA | NA |
| | | | | | 179296823 | | | |
| PRKRIP1 | NA | 510.496 | −4.789046 | uc003uzh.2 | chr7: 102065559- | NA | NA | NA |
| | | | | | 102067129 | | | |
| PRKRIR | 607374 | NA | NA | NA | NA | 620.335 | −6.706162 | uc001oxh.1 |
| PRKX | 300083 | NA | NA | NA | NA | 237.423 | −7.296506 | uc010nde.3 |
| PRL | 176760 | NA | NA | NA | NA | 738.589 | −7.126055 | uc003ndo.3 |
| PRLHR | 600895 | 305.178 | −4.590193 | uc001ldp.1 | chr10: 120352916- | NA | NA | NA |
| | | | | | 120353643 | | | |
| PRMT2 | 601961 | 680.167 | −4.650683 | uc002zjw.3 | chr21: 48078837- | NA | NA | NA |
| | | | | | 48080068 | | | |
| PRMT3 | 603190 | NA | NA | NA | NA | 644.578 | −9.073898 | uc001mqb.3 |
| PRNP | 176640 | NA | NA | NA | NA | 896.068 | −10.343675 | uc002wkt.1 |
| PROM1 | 604365 | 593.708 | −7.141342 | uc003goo.2 | chr4: 15969849- | NA | NA | NA |
| | | | | | 15970991 | | | |
| PROM2 | NA | NA | NA | NA | NA | 581.937 | −10.611033 | uc002suh.2 |
| PROS1 | 176880 | 508.807 | −7.65441 | uc010hoo.3 | chr3: 93591881- | NA | NA | NA |
| | | | | | 93593088 | | | |
| PROSER3 | NA | NA | NA | NA | NA | NA | NA | NA |
| PRPF18 | 604993 | NA | NA | NA | NA | NA | NA | NA |
| PRPF38A | NA | NA | NA | NA | NA | 432.619 | −5.708473 | uc001ctv.4 |
| PRPF39 | 614907 | 1196.34 | −8.548215 | uc001wvy.4 | chr14: 45584117- | NA | NA | NA |
| | | | | | 45584804 | | | |
| PRPF8 | 607300 | 615.088 | −6.450075 | uc002fte.3 | chr17: 1553923- | NA | NA | NA |
| | | | | | 1554095 | | | |
| PRPS2 | 311860 | NA | NA | NA | NA | NA | NA | NA |
| PRR14 | NA | 1298.36 | −9.874897 | uc002dyy.3 | chr16: 30667633- | 685.632 | −7.169206 | uc002dyy.3 |
| | | | | | 30667734 | | | |
| PRR14L | NA | 1647.72 | −12.951215 | uc003alp.4 | chr22: 32077334- | 1646.21 | −2.465276 | uc003alp.4 |
| | | | | | 32081512 | | | |
| PRR16 | 615931 | NA | NA | NA | NA | NA | NA | NA |
| PRR23A | NA | NA | NA | NA | NA | NA | NA | NA |
| PRR23B | NA | NA | NA | NA | NA | NA | NA | NA |
| PRR23C | NA | 711.16 | −7.999624 | uc011bmt.1 | chr3: 138760944- | NA | NA | NA |
| | | | | | 138762673 | | | |
| PRR26 | NA | NA | NA | NA | NA | NA | NA | NA |
| PRR27 | NA | NA | NA | NA | NA | NA | NA | NA |
| PRR3 | NA | 493.769 | −4.890177 | uc003nqi.2 | chr6: 30530273- | NA | NA | NA |
| | | | | | 30532473 | | | |
| PRR32 | NA | 473.456 | −8.68197 | uc010nra.3 | chrX: 125955519- | NA | NA | NA |
| | | | | | 125955768 | | | |
| PRR34 | NA | 780.67 | −6.100478 | uc003bgq.1 | chr22: 46446339- | NA | NA | NA |
| | | | | | 46447706 | | | |
| PRR9 | NA | NA | NA | NA | NA | 521.659 | −7.316097 | uc021ozw.1 |
| PRRC1 | NA | 1443.46 | −7.643477 | uc003kuj.4 | chr5: 126883743- | NA | NA | NA |
| | | | | | 126887834 | | | |
| PRRG1 | 300935 | 1637.02 | −9.643232 | uc022but.1 | chrX: 37300122- | NA | NA | NA |
| | | | | | 37301477 | | | |
| PRRG3 | 300685 | NA | NA | NA | NA | NA | NA | NA |
| PRRT1 | NA | NA | NA | NA | NA | 1126.71 | −4.780384 | uc003nzs.3 |
| PRRT2 | 614386 | 578.343 | −4.048622 | uc002due.4 | chr16: 29825960- | NA | NA | NA |
| | | | | | 29827202 | | | |
| PRSS22 | 609343 | NA | NA | NA | NA | NA | NA | NA |
| PRSS23 | NA | 492.913 | −5.607231 | uc010rts.1 | chr11: 86519838- | NA | NA | NA |
| | | | | | 86520169 | | | |
| PRSS35 | NA | NA | NA | NA | NA | 248.079 | −8.39324 | uc003pjz.3 |
| PRSS42 | NA | NA | NA | NA | NA | 393.69 | −7.07113 | uc003cqj.3 |
| PRSS45 | NA | 393.444 | −4.350072 | uc010hjl.3 | chr3: 46783581- | NA | NA | NA |
| | | | | | 46783839 | | | |
| PRSS55 | 615144 | NA | NA | NA | NA | 473.422 | −4.859588 | uc022art.1 |
| PRTG | 613261 | NA | NA | NA | NA | NA | NA | NA |
| PRUNE2 | 610691 | NA | NA | NA | NA | 755.806 | −9.052608 | uc011lsk.2 |
| PRX | 605725 | NA | NA | NA | NA | 590.453 | −3.924999 | uc002onq.3 |
| PSAP | 176801 | NA | NA | NA | NA | 276.587 | −4.393927 | uc001jsm.3 |
| PSD3 | 614440 | 510.399 | −8.67216 | uc003wyx.4 | chr8: 18384813- | NA | NA | NA |
| | | | | | 18393252 | | | |
| PSEN2 | 600759 | 575.096 | −9.08846 | uc009xeo.1 | chr1: 227083281- | NA | NA | NA |
| | | | | | 227083804 | | | |
| PSG11 | 176401 | NA | NA | NA | NA | 499.226 | −7.759387 | NA |
| PSG3 | 176392 | NA | NA | NA | NA | 322.983 | −6.990292 | uc002oue.3 |
| PSG9 | 176398 | 275.409 | −5.296089 | uc002owg.2 | chr19: 43761312- | NA | NA | NA |
| | | | | | 43762108 | | | |
| PSMA1 | 602854 | NA | NA | NA | NA | NA | NA | NA |
| PSMA2 | 176842 | NA | NA | NA | NA | NA | NA | NA |
| PSMA3 | 176843 | NA | NA | NA | NA | 534.915 | −7.582176 | uc001xdj.2 |
| PSMA4 | 176846 | NA | NA | NA | NA | 763.384 | −7.228475 | uc002bdu.4 |
| PSMA5 | 176844 | NA | NA | NA | NA | NA | NA | NA |
| PSMA6 | 602855 | 257.156 | −4.915418 | uc001wtd.3 | chr14: 35786513- | NA | NA | NA |
| | | | | | 35786682 | | | |
| PSMB1 | 602017 | 1063.58 | −9.550596 | uc003qxr.3 | chr6: 170844204- | NA | NA | NA |
| | | | | | 170844307 | | | |
| PSMB2 | 602175 | NA | NA | NA | NA | 1093.51 | −7.113083 | uc001bzd.2 |
| PSMB3 | 602176 | 581.496 | −4.937854 | uc002hqr.3 | chr17: 36920421- | NA | NA | NA |
| | | | | | 36920478 | | | |
| PSMB4 | 602177 | 426.102 | −5.345978 | uc010pda.2 | chr1: 151373075- | NA | NA | NA |
| | | | | | 151373603 | | | |
| PSMB5 | 600306 | 788.485 | −7.900773 | uc001wii.3 | chr14: 23495060- | 994.85 | −9.890269 | uc001wii.3 |
| | | | | | 23495297 | | | |
| PSMB9 | 177045 | NA | NA | NA | NA | 328.886 | −3.3351 | uc011dqj.2 |
| PSMC6 | 602708 | 1049 | −10.965282 | uc010tqv.2 | chr14: 53176326- | NA | NA | NA |
| | | | | | 53177053 | | | |

| | | Frame 3 | | | |
|---|---|---|---|---|---|
| | Frame 2 | | | ucSC | |
| Gene | Hg19 Location | TANGO | PASTA | Transcript ID | Hg19 Location |
| A2M | chr12: 9220304- | NA | NA | NA | NA |
| | 9220418 | | | | |
| AACS | chr12: 125626776- | 481.525 | −8.254097 | uc001uhc.3 | chr12: 125626776- |
| | 125627871 | | | | 125627871 |
| AADACL3 | NA | 729.366 | −6.520575 | uc001aug.1 | chr1: 12785964- |
| | | | | | 12788726 |
| AAED1 | NA | NA | NA | NA | NA |
| AAK1 | chr2: 69771371- | NA | NA | NA | NA |
| | 69771526 | | | | |
| AARS | NA | 331.065 | −4.734393 | uc010vlu.1 | chr16: 70286297- |
| | | | | | 70286623 |
| AARSD1 | chr17: 41102543- | NA | NA | NA | NA |
| | 41102609 | | | | |
| AASDH | NA | 1407.39 | −12.190286 | NA | NA |
| AASS | chr7: 121713598- | 500.27 | −4.941699 | uc003vka.3 | chr7: 121713598- |
| | 121716542 | | | | 121716542 |
| ABCA1 | chr9: 107543284- | 820.407 | −4.653754 | uc004bcl.3 | chr9: 107543284- |
| | 107546595 | | | | 107546595 |
| ABCA10 | chr17: 67144148- | NA | NA | NA | NA |
| | 67144967 | | | | |
| ABCA13 | NA | NA | NA | NA | NA |
| ABCA3 | chr16: 2325879- | NA | NA | NA | NA |
| | 2326674 | | | | |
| ABCA5 | chr17: 67240576- | 663.697 | −6.410467 | uc002jib.2 | chr17: 67240576- |
| | 67243677 | | | | 67243677 |
| ABCA6 | chr17: 67131365- | NA | NA | NA | NA |
| | 67132134 | | | | |
| ABCA8 | chr17: 66863431- | 1461.34 | −10.227365 | uc002jhp.3 | chr17: 66863431- |
| | 66864225 | | | | 66864225 |
| ABCA9 | chr17: 66970773- | NA | NA | NA | NA |
| | 66972055 | | | | |
| ABCB11 | NA | NA | NA | NA | NA |
| ABGB4 | chr7: 87081712- | NA | NA | NA | NA |
| | 87082255 | | | | |
| ABGB5 | NA | NA | NA | NA | NA |
| ABCB6 | NA | 814.821 | −6.721982 | uc002vkc.2 | chr2: 220074488- |
| | | | | | 220074659 |
| ABCC2 | chr10: 101611389- | NA | NA | NA | NA |
| | 101611662 | | | | |
| ABCC3 | NA | 449.528 | −6.51192 | uc002isl.3 | chr17: 48768562- |
| | | | | | 48769063 |
| ABCC4 | NA | NA | NA | NA | NA |
| ABCC5 | NA | 294.71 | −1.424308 | uc003fmh.3 | chr3: 183701541- |
| | | | | | 183702682 |
| ABCG2 | NA | 1424.91 | −14.381849 | uc003hrf.3 | chr4: 89011416- |
| | | | | | 89013385 |
| ABHD12B | NA | 566.71 | −7.528861 | uc001wyq.3 | chr14: 51371085- |
| | | | | | 51371688 |
| ABHD13 | NA | 1320.81 | −9.520672 | uc001vqq.3 | chr13: 108882581- |
| | | | | | 108886603 |
| ABHD16A | NA | 657.55 | −4.909684 | uc003nvx.2 | chr6: 31654726- |
| | | | | | 31654988 |
| ABHD17B | NA | 378.643 | −4.252365 | uc004ail.3 | chr9: 74477368- |
| | | | | | 74477496 |
| ABHD3 | NA | 749.093 | −10.060514 | uc002ktk.2 | chr18: 19230858- |
| | | | | | 19231551 |
| ABI1 | chr10: 27035525- | NA | NA | NA | NA |
| | 27037498 | | | | |
| ABI3BP | NA | NA | NA | NA | NA |
| ABL2 | chr1: 179079716- | NA | NA | NA | NA |
| | 179079939 | | | | |
| ABLIM3 | chr5: 148637968- | NA | NA | NA | NA |
| | 148639999 | | | | |
| ABRACL | chr6: 139364041- | 536.08 | −6.256734 | uc003qil.2 | chr6: 139364041- |
| | 139364439 | | | | 139364439 |
| ABT1 | chr6: 26598874- | NA | NA | NA | NA |
| | 26600277 | | | | |
| ABTB1 | NA | 948.29 | −8.83878 | uc003ejr.3 | chr3: 127399319- |
| | | | | | 127399769 |
| ACAD10 | chr12: 112147510- | NA | NA | NA | NA |
| | 112148121 | | | | |
| ACAD8 | chr11: 134129742- | 486.637 | −5.57642 | uc009zdc.3 | chr11: 134129078- |
| | 134131073 | | | | 134129819 |
| ACAD9 | chr3: 128631451- | 451.749 | −2.073016 | uc010hsw.1 | chr3: 128627112- |
| | 128631957 | | | | 128627942 |
| ACAT1 | chr11: 108006851- | 1111.32 | −8.330367 | uc001pjw.1 | chr11: 108006851- |
| | 108007666 | | | | 108007666 |
| ACBD3 | NA | 672.713 | −7.216864 | uc001hpy.3 | chr1: 226332380- |
| | | | | | 226334310 |
| ACBD5 | NA | 503.216 | −8.008755 | uc010qdp.2 | chr10: 27484143- |
| | | | | | 27486358 |
| ACCS | NA | 226.938 | −7.053404 | uc009yks.1 | chr11: 44105343- |
| | | | | | 44105569 |
| ACD | NA | 373.642 | −4.966819 | uc002etp.4 | chr16: 67691415- |
| | | | | | 67691508 |
| ACE2 | chrX: 15579156- | NA | NA | NA | NA |
| | 15580027 | | | | |
| ACER2 | NA | 334.797 | −4.878652 | uc003zny.1 | chr9: 19450635- |
| | | | | | 19452500 |
| ACER3 | NA | NA | NA | NA | NA |
| ACHE | chr7: 100487615- | NA | NA | NA | NA |
| | 100487834 | | | | |
| ACKR3 | NA | 286.297 | −5.752442 | uc010fyq.3 | chr2: 237490198- |
| | | | | | 237490994 |
| ACKR4 | NA | 503.554 | −5.590682 | uc003eow.3 | chr3: 132320295- |
| | | | | | 132321382 |
| ACMSD | NA | 618.647 | −5.189157 | uc002ttz.3 | chr2: 135659431- |
| | | | | | 135659602 |
| ACO1 | chr9: 32436141- | 1214.08 | −5.439037 | uc010mjh.1 | chr9: 32436141- |
| | 32436395 | | | | 32436395 |
| ACOT1 | NA | NA | NA | NA | NA |
| ACOT8 | NA | 855.458 | −5.407401 | uc002xqa.2 | chr20: 44470360- |
| | | | | | 44470476 |
| ACOT9 | NA | 536.376 | −4.436186 | uc004dan.3 | chrX: 23721777- |
| | | | | | 23722010 |
| ACOX2 | NA | NA | NA | NA | NA |
| ACP5 | NA | 707.175 | −6.930743 | uc002msg.4 | chr19: 11685475- |
| | | | | | 11585824 |
| ACRC | NA | NA | NA | NA | NA |
| ACRV1 | NA | 677.655 | −5.115082 | uc001qcl.3 | chr11: 125542229- |
| | | | | | 125542487 |
| ACSL3 | NA | 748.953 | −6.110405 | uc002vni.3 | chr2: 223806373- |
| | | | | | 223808119 |
| ACSL4 | NA | NA | NA | NA | NA |
| ACSL6 | NA | 485.625 | −8.678555 | uc003kvw.2 | chr5: 131285667- |
| | | | | | 131289926 |
| ACSM3 | chr16: 20808295- | 219.8 | −6.470872 | uc002dhq.3 | chr16: 20797574- |
| | 20808479 | | | | 20797638 |
| ACSM6 | NA | 482.704 | −6.205442 | uc001kke.3 | chr10: 96988542- |
| | | | | | 96988686 |
| ACSS1 | chr20: 24986866- | NA | NA | NA | NA |
| | 24988397 | | | | |
| ACTA1 | chr1: 229566993- | NA | NA | NA | NA |
| | 229567245 | | | | |
| ACTA2 | NA | NA | NA | NA | NA |
| ACTBL2 | NA | NA | NA | NA | NA |
| ACTC1 | NA | NA | NA | NA | NA |
| ACTG1 | NA | 741.063 | −4.367605 | uc002kak.2 | chr17: 79476997- |
| | | | | | 79477715 |
| ACTL6A | chr3: 179305799- | NA | NA | NA | NA |
| | 179306193 | | | | |
| ACTL8 | NA | NA | NA | NA | NA |
| ACTN2 | NA | 985.91 | −6.298311 | uc001hyf.2 | chr1: 236925920- |
| | | | | | 236927558 |
| ACTR1B | NA | NA | NA | NA | NA |
| ACTR2 | NA | NA | NA | NA | NA |
| ACTR3C | chr7: 149944301- | NA | NA | NA | NA |
| | 149944658 | | | | |
| ACTRT1 | NA | 267.576 | −3.690155 | NA | NA |
| ACTRT3 | chr3: 169484711- | NA | NA | NA | NA |
| | 169485219 | | | | |
| ACVR1B | NA | 364.32 | −7.824208 | uc001rzl.3 | chr12: 52380930- |
| | | | | | 52381130 |
| ACVR1C | chr2: 158383279- | NA | NA | NA | NA |
| | 158390429 | | | | |
| ACY1 | chr3: 52021662- | 1077.04 | −8.009561 | uc011beb.2 | chr3: 52021662- |
| | 52022246 | | | | 52022246 |
| ACYP1 | NA | NA | NA | NA | NA |
| ADA | NA | 431.54 | −6.542354 | uc002xmj.3 | chr20: 43248163- |
| | | | | | 43248474 |
| ADAM10 | NA | 817.057 | −14.861574 | uc002afd.1 | chr15: 58888510- |
| | | | | | 58889745 |
| ADAM18 | NA | 390.347 | −5.117055 | uc003xni.3 | chr8: 39587460- |
| | | | | | 39587583 |
| ADAM21 | NA | NA | NA | NA | NA |
| ADAM33 | chr20: 3653368- | 1408.03 | −9.51681 | uc010zqg.1 | chr20: 3653368- |
| | 3653960 | | | | 3653960 |
| ADAM9 | chr8: 38871617- | 378.947 | −4.261875 | uc003xmr.3 | chr8: 38961220- |
| | 38873537 | | | | 38962779 |
| ADAMTS12 | NA | 1223.63 | −8.112914 | uc003jib.1 | chr5: 33751303- |
| | | | | | 33751443 |
| ADAMTS14 | NA | NA | NA | NA | NA |
| ADAMTS16 | chr5: 5319252- | NA | NA | NA | NA |
| | 5320412 | | | | |
| ADAMTS19 | NA | 773.002 | −9.81295 | uc003kvb.1 | chr5: 129072912- |
| | | | | | 129074376 |
| ADAMTS2 | NA | 593.284 | −8.032295 | uc003mjw.3 | chr5: 178537852- |
| | | | | | 178540867 |
| ADAMTS20 | chr12: 43816622- | NA | NA | NA | NA |
| | 43817496 | | | | |
| ADAMTS5 | NA | NA | NA | NA | NA |
| ADAMTS6 | NA | 1182.65 | −7.401157 | uc003jtr.1 | chr5: 64510225- |
| | | | | | 64510468 |
| ADAMTS9 | chr3: 64507157- | NA | NA | NA | NA |
| | 64507344 | | | | |
| ADAMTSL1 | NA | 793.966 | −8.088577 | uc003znc.4 | chr9: 18684803- |
| | | | | | 18684952 |
| ADAMTSL4 | NA | 555.351 | −5.567336 | uc001eux.3 | chr1: 150532673- |
| | | | | | 150533412 |
| ADAT1 | chr16: 75632247- | NA | NA | NA | NA |
| | 75634113 | | | | |
| ADCY10 | chr1: 167778625- | NA | NA | NA | NA |
| | 167778914 | | | | |
| ADCY2 | NA | NA | NA | NA | NA |
| ADCY8 | chr8: 131792547- | 1180.96 | −9.226396 | uc003ytd.4 | chr8: 131792547- |
| | 131792635 | | | | 131792635 |
| ADCY9 | NA | NA | NA | NA | NA |
| ADGB | chr6: 147136354- | NA | NA | NA | NA |
| | 147136597 | | | | |
| ADGRE3 | chr19: 14730051- | NA | NA | NA | NA |
| | 14730244 | | | | |
| ADGRF2 | chr6: 47662726- | NA | NA | NA | NA |
| | 47665533 | | | | |
| ADGRG3 | NA | NA | NA | NA | NA |
| ADGRG5 | NA | 650.891 | −6.042059 | uc002elx.4 | chr16: 57609451- |
| | | | | | 57611100 |
| ADGRL3 | NA | 356.231 | −4.435821 | uc003hcq.4 | chr4: 62935888- |
| | | | | | 62938168 |
| ADGRL4 | chr1: 79355449- | NA | NA | NA | NA |
| | 79356838 | | | | |
| ADH1A | chr4: 100197523- | NA | NA | NA | NA |
| | 100197780 | | | | |
| ADH1B | chr4: 100227527- | NA | NA | NA | NA |
| | 100228996 | | | | |
| ADH4 | chr4: 100044833- | NA | NA | NA | NA |
| | 100045592 | | | | |
| ADH5 | NA | NA | NA | NA | NA |
| ADH6 | NA | 705.212 | −4.299435 | uc003hup.4 | chr4: 100125879- |
| | | | | | 100126077 |
| ADH7 | NA | NA | NA | NA | NA |
| ADI1 | NA | NA | NA | NA | NA |
| ADK | NA | 447.739 | −7.909151 | uc001jwi.3 | chr10: 76468204- |
| | | | | | 76469061 |
| ADORA2B | NA | NA | NA | NA | NA |
| ADORA3 | NA | 612.767 | −6.035027 | uc001ebf.3 | ch1: 112025970- |
| | | | | | 112026308 |
| ADPGK | NA | 487.965 | −5.933672 | uc002ave.4 | chr15: 73043708- |
| | | | | | 73044681 |
| ADPRH | NA | NA | NA | NA | NA |
| ADPRM | chr17: 10614462- | NA | NA | NA | NA |
| | 10614875 | | | | |
| ADRA1A | chr8: 26605667- | NA | NA | NA | NA |
| | 26606106 | | | | |
| ADRB2 | chr5: 148207637- | NA | NA | NA | NA |
| | 148208197 | | | | |
| ADRBK1 | NA | 499.662 | −9.620672 | uc009yrn.1 | chr11: 67052922- |
| | | | | | 67054029 |
| ADTRP | chr6: 11713888- | 674.969 | −7.215103 | uc003nab.3 | chr6: 11713888- |
| | 11714710 | | | | 11714710 |
| AEBP2 | NA | NA | NA | NA | NA |
| AEN | chr15: 89170047- | NA | NA | NA | NA |
| | 89170572 | | | | |
| AFAP1L2 | NA | 1158.34 | −5.451382 | uc001lbm.3 | chr10: 116054583- |
| | | | | | 116055800 |
| AFF1 | NA | 782.31 | −8.587524 | uc011ccx.2 | chr4: 87968840- |
| | | | | | 87969201 |
| AFF2 | chrX: 147985793- | NA | NA | NA | NA |
| | 147985907 | | | | |
| AFG3L2 | chr18: 12328943- | 579.044 | −5.114649 | uc002kqz.2 | chr18: 12328943- |
| | 12329563 | | | | 12329563 |
| AFMID | NA | 669.618 | −8.098637 | uc002jvb.3 | chr17: 76202992- |
| | | | | | 76203782 |
| AFP | NA | NA | NA | NA | NA |
| AGA | NA | 471.495 | −6.301065 | uc003iuu.2 | chr4: 178351929- |
| | | | | | 178352861 |
| AGAP6 | NA | NA | NA | NA | NA |
| AGAP9 | chr10: 48237211- | NA | NA | NA | NA |
| | 48237508 | | | | |
| AGBL2 | NA | 601.081 | −5.305395 | uc001ngg.3 | chr11: 47681143- |
| | | | | | 47681724 |
| AGFG1 | NA | 1252.94 | −11.456195 | uc002vpc.2 | chr2: 228419212- |
| | | | | | 228425938 |
| AGGF1 | chr5: 76331584- | NA | NA | NA | NA |
| | 76331878 | | | | |
| AGL | chr1: 100387208- | NA | NA | NA | NA |
| | 100389579 | | | | |
| AGO2 | NA | NA | NA | NA | NA |
| AGPAT1 | NA | 371.349 | −4.487595 | uc003oae.3 | chr6: 32135983- |
| | | | | | 32137052 |
| AGPAT5 | chr8: 6614910- | NA | NA | NA | NA |
| | 6619021 | | | | |
| AGPS | NA | 1632.53 | −20.833164 | uc002ull.2 | chr2: 178402924- |
| | | | | | 178408564 |
| AGR2 | chr7: 16832264- | NA | NA | NA | NA |
| | 16832531 | | | | |
| AGRN | chr1: 990362- | 1146.18 | −10.042644 | uc001ack.2 | chr1: 990362- |
| | 991499 | | | | 991499 |
| AGTRAP | chr1: 11810162- | NA | NA | NA | NA |
| | 11810828 | | | | |
| AHCTF1 | NA | NA | NA | NA | NA |
| AHR | NA | 626.851 | −6.460146 | uc011jxz.1 | chr7: 17382689- |
| | | | | | 17385775 |
| AHSA2 | NA | NA | NA | NA | NA |
| AHSG | chr3: 186335305- | NA | NA | NA | NA |
| | 186335490 | | | | |
| AIFM2 | NA | NA | NA | NA | NA |
| AIM1L | NA | NA | NA | NA | NA |
| AIMP1 | chr4: 107268850- | 477.99 | −6.827829 | uc011cfg.2 | chr4: 107268850- |
| | 107270381 | | | | 107270381 |
| AK1 | NA | 582.029 | −4.691259 | uc004bsm.4 | chr9: 130628759- |
| | | | | | 130630286 |
| AK4 | chr1: 65691861- | NA | NA | NA | NA |
| | 65697828 | | | | |
| AK9 | chr6: 109952900- | 1756.19 | −24.936199 | uc011eas.1 | chr6: 109814059- |
| | 109954113 | | | | 109814571 |
| AKAP1 | NA | NA | NA | NA | NA |
| AKAP11 | NA | 682.059 | −8.962048 | uc001uys.2 | chr13: 42893365- |
| | | | | | 42897403 |
| AKAP12 | NA | 444.528 | −5.560655 | uc011eep.2 | chr6: 151676862- |
| | | | | | 151679694 |
| AKAP14 | NA | 446.164 | −8.318573 | uc004ese.3 | chrX: 119054571- |
| | | | | | 119054679 |
| AKAP5 | NA | NA | NA | NA | NA |
| AKAP6 | chr14: 33201888- | 536.53 | −9.371549 | uc010aml.3 | chr14: 33201888- |
| | 33203819 | | | | 33203819 |
| AKAP9 | chr7: 91670228- | 565.947 | −7.550122 | uc003ule.2 | chr7: 91670228- |
| | 91671095 | | | | 91671095 |
| AKR1B10 | NA | 284.286 | −4.559926 | uc003vrr.3 | chr7: 134225842- |
| | | | | | 134226166 |
| AKR1B15 | chr7: 134264302- | NA | NA | NA | NA |
| | 134264592 | | | | |
| AKR1C3 | NA | NA | NA | NA | NA |
| AKR7A2 | chr1: 19629202- | NA | NA | NA | NA |
| | 19630718 | | | | |
| AKR7A3 | chr1: 19609057- | NA | NA | NA | NA |
| | 19609224 | | | | |
| AKT3 | chr1: 243663021- | NA | NA | NA | NA |
| | 243668550 | | | | |
| ALB | NA | 841.842 | −6.89653 | uc003hgs.4 | chr4: 74286809- |
| | | | | | 74287129 |
| ALDH1A1 | chr9: 75515578- | NA | NA | NA | NA |
| | 75516175 | | | | |
| ALDH3A2 | NA | NA | NA | NA | NA |
| ALDH6A1 | NA | 605.44 | −4.749762 | uc001xpo.3 | chr14: 74526872- |
| | | | | | 74527344 |
| ALDOC | NA | NA | NA | NA | NA |
| ALG13 | chrX: 110931115- | 896.348 | −8.461835 | uc022ccn.1 | chrX: 110931115- |
| | 110933623 | | | | 110933623 |
| ALG14 | NA | NA | NA | NA | NA |
| ALG2 | chr9: 101978707- | 626.239 | −6.793333 | uc004azf.3 | chr9: 101978707- |
| | 101980215 | | | | 101980215 |
| ALG5 | chr13: 37523908- | 990.773 | −11.701294 | uc001uvy.3 | chr13: 37523908- |
| | 37524078 | | | | 37524078 |
| ALG9 | chr11: 111652919- | 937.226 | −5.064702 | uc001ply.3 | chr11: 111652919- |
| | 111657120 | | | | 111657120 |
| ALKBH5 | NA | 1601.92 | −15.166409 | uc010cpw.3 | chr17: 18111711- |
| | | | | | 18113267 |
| ALLC | NA | 617.834 | −10.792376 | uc010ewt.3 | chr2: 3750154- |
| | | | | | 3750260 |
| ALMS1P | chr2: 73912218- | NA | NA | NA | NA |
| | 73912694 | | | | |
| ALOX5AP | chr13: 31338244- | NA | NA | NA | NA |
| | 31338565 | | | | |
| ALS2 | chr2: 202564986- | NA | NA | NA | NA |
| | 202566573 | | | | |
| ALS2CR12 | NA | 736.299 | −5.560655 | uc002uya.4 | chr2: 202153147- |
| | | | | | 202153377 |
| ALX1 | chr12: 85695254- | NA | NA | NA | NA |
| | 85695561 | | | | |
| AMACR | chr5: 33987091- | NA | NA | NA | NA |
| | 33989197 | | | | |
| AMBN | chr4: 71472448- | NA | NA | NA | NA |
| | 71473004 | | | | |
| AMBP | NA | 581.029 | −7.381495 | uc004bie.4 | chr9: 116822408- |
| | | | | | 116822518 |
| AMELY | chrY: 6733959- | NA | NA | NA | NA |
| | 6734113 | | | | |
| AMICA1 | NA | NA | NA | NA | NA |
| AMMECR1 | chrX: 109437414- | NA | NA | NA | NA |
| | 109441747 | | | | |
| AMMECR1L | chr2: 128619207- | NA | NA | NA | NA |
| | 128622667 | | | | |
| AMN1 | chr12: 31824071- | NA | NA | NA | NA |
| | 31825237 | | | | |
| AMOT1 | chrX: 112018105- | NA | NA | NA | NA |
| | 112021794 | | | | |
| AMPD2 | NA | 1543.08 | −10.475448 | uc010ovr.1 | chr1: 110173775- |
| | | | | | 110174677 |
| AMPD3 | NA | 305.567 | −4.895548 | uc010rbz.1 | chr11: 10527432- |
| | | | | | 10529126 |
| AMTN | NA | 712.359 | −6.576645 | uc003hfk.1 | chr4: 71398183- |
| | | | | | 71398459 |
| ANG | chr14: 21162168- | 784.738 | −5.646233 | uc001vxw.4 | chr14: 21162168- |
| | 21162345 | | | | 21162345 |
| ANGEL2 | NA | 388.44 | −5.09284 | uc010pto.2 | chr1: 213165524- |
| | | | | | 213168382 |
| ANGPT1 | chr8: 108261710- | NA | NA | NA | NA |
| | 108264082 | | | | |
| ANGPT2 | chr8: 6357175- | NA | NA | NA | NA |
| | 6360621 | | | | |
| ANHX | NA | NA | NA | NA | NA |
| ANKH | chr5: 14704909- | NA | NA | NA | NA |
| | 14711305 | | | | |
| ANKHD1 | chr5: 139919004- | NA | NA | NA | NA |
| | 139919441 | | | | |
| ANKMY1 | NA | NA | NA | NA | NA |
| ANKRA2 | NA | NA | NA | NA | NA |
| ANKRD11 | NA | 455.414 | −4.355335 | uc002fnf.1 | chr16: 89365283- |
| | | | | | 89367221 |
| ANKRD13A | chr12: 110458965- | NA | NA | NA | NA |
| | 110460255 | | | | |
| ANKRD17 | chr4: 73940502- | 261.047 | −5.135784 | uc003hgo.3 | chr4: 73940502- |
| | 73941947 | | | | 73941947 |
| ANKRD20A5P | chr18: 14184108- | NA | NA | NA | NA |
| | 14185386 | | | | |
| ANKRD26 | NA | 1406.11 | −8.665963 | uc001itg.2 | chr10: 27293045- |
| | | | | | 27294518 |
| ANKRD28 | NA | NA | NA | NA | NA |
| ANKRD31 | NA | 652.133 | −9.955255 | uc003kdo.2 | chr5: 74364122- |
| | | | | | 74364343 |
| ANKRD34C | NA | 235.199 | −8.365459 | uc002bet.3 | chr15: 79587235- |
| | | | | | 79590581 |
| ANKRD40 | NA | 700.046 | −7.298207 | uc002iso.3 | chr17: 48770551- |
| | | | | | 48773357 |
| ANKRD42 | NA | 231.68 | −3.64839 | uc009yvi.2 | chr11: 82923706- |
| | | | | | 82926242 |
| ANKRD44 | NA | 1279.16 | −6.026144 | uc002utz.4 | chr2: 197831741- |
| | | | | | 197832162 |
| ANKRD45 | chr1: 173577475- | NA | NA | NA | NA |
| | 173579283 | | | | |
| ANKRD49 | chr11: 94231699- | NA | NA | NA | NA |
| | 94232744 | | | | |
| ANKRD50 | chr4: 125585204- | NA | NA | NA | NA |
| | 125588669 | | | | |
| ANKRD6 | NA | 390.278 | −6.46399 | uc003pne.4 | chr6: 90340724- |
| | | | | | 90343553 |
| ANKRD7 | chr7: 117882403- | 285.108 | −3.450736 | uc003vji.3 | chr7: 117882403- |
| | 117882784 | | | | 117882784 |
| ANKS4B | NA | NA | NA | NA | NA |
| ANO1 | chr11: 70034111- | NA | NA | NA | NA |
| | 70035652 | | | | |
| ANO4 | NA | 777.749 | −5.490211 | uc001thw.2 | chr12: 101521639- |
| | | | | | 101522419 |
| ANO5 | chr11: 22301312- | NA | NA | NA | NA |
| | 22304913 | | | | |
| ANO6 | NA | 2040.2 | −9.488139 | uc001roo.3 | chr12: 45823095- |
| | | | | | 45826134 |
| ANO7 | chr2: 242163569- | NA | NA | NA | NA |
| | 242164791 | | | | |
| ANP32B | chr9: 100777714- | NA | NA | NA | NA |
| | 100778224 | | | | |
| ANXA1 | NA | 501.654 | −6.105755 | uc004ajf.1 | chr9: 75785024- |
| | | | | | 75785307 |
| ANXA3 | NA | 1691.93 | −10.747866 | uc003hld.3 | chr4: 79531270- |
| | | | | | 79531605 |
| ANXA5 | chr4: 122589152- | NA | NA | NA | NA |
| | 122589622 | | | | |
| ANXA7 | chr10: 75135189- | 513.002 | −5.575595 | uc001jtz.2 | chr10: 75135189- |
| | 75135852 | | | | 75135852 |
| AOC2 | chr17: 41002366- | NA | NA | NA | NA |
| | 41002724 | | | | |
| AOX1 | NA | 516.271 | −4.050784 | uc002uvx.3 | chr2: 201535403- |
| | | | | | 201536217 |
| AP1G1 | NA | NA | NA | NA | NA |
| AP2A2 | chr11: 1010626- | NA | NA | NA | NA |
| | 1012245 | | | | |
| AP3M1 | NA | NA | NA | NA | NA |
| AP4E1 | NA | 282.664 | −4.926208 | uc001zyx.2 | chr15: 51294860- |
| | | | | | 51298097 |
| AP4S1 | NA | 271.002 | −4.681 | uc001wqw.4 | chr14: 31554089- |
| | | | | | 31555008 |
| AP5M1 | chr14: 57741743- | 1089.82 | −15.622121 | uc001xcu.4 | chr14: 57741743- |
| | 57742175 | | | | 57742175 |
| AP5S1 | NA | NA | NA | NA | NA |
| APAF1 | chr12: 99126260- | NA | NA | NA | NA |
| | 99129211 | | | | |
| APBA1 | chr9: 72042449- | NA | NA | NA | NA |
| | 72046242 | | | | |
| APBA2 | chr15: 29394123- | 806.637 | −12.058342 | uc001zcm.1 | chr15: 29394123- |
| | 29394962 | | | | 29394962 |
| APBA3 | chr19: 3750771- | NA | NA | NA | NA |
| | 3751023 | | | | |
| APBB1IP | NA | NA | NA | NA | NA |
| APEH | NA | NA | NA | NA | NA |
| API5 | NA | 1139.47 | −10.786537 | uc001mxf.2 | chr11: 43364006- |
| | | | | | 43366080 |
| APIP | chr11: 34896714- | NA | NA | NA | NA |
| | 34896830 | | | | |
| APLP2 | NA | 631.171 | −7.164445 | uc001qfp.3 | chr11: 130013344- |
| | | | | | 130014706 |
| APOA5 | chr11: 116660086- | NA | NA | NA | NA |
| | 116660843 | | | | |
| APOB | chr2: 21224301- | NA | NA | NA | NA |
| | 21224601 | | | | |
| APOBEC3C | NA | NA | NA | NA | NA |
| APOE | NA | 301.876 | −5.92551 | uc002pab.3 | chr19: 45412508- |
| | | | | | 45412650 |
| APOH | chr17: 64208147- | NA | NA | NA | NA |
| | 64208250 | | | | |
| APOL2 | NA | NA | NA | NA | NA |
| APPL1 | NA | NA | NA | NA | NA |
| AQP10 | NA | NA | NA | NA | NA |
| AQP9 | chr15: 58476335- | NA | NA | NA | NA |
| | 58478110 | | | | |
| AQR | chr15: 35148552- | NA | NA | NA | NA |
| | 35148992 | | | | |
| ARAP2 | NA | 379.732 | −5.527587 | uc003gsq.2 | chr4: 36067620- |
| | | | | | 36069528 |
| ARF1 | NA | 766.008 | −4.854909 | uc001hrr.3 | chr1: 228285715- |
| | | | | | 228286913 |
| ARF6 | chr14: 50360983- | NA | NA | NA | NA |
| | 50363772 | | | | |
| ARFGAP1 | NA | 332.536 | −4.109288 | uc011aas.1 | chr20: 61919226- |
| | | | | | 61920848 |
| ARFGAP2 | NA | 510.89 | −6.793713 | uc010rhd.2 | chr11: 47194649- |
| | | | | | 47194866 |
| ARFGEF1 | chr8: 68109884- | NA | NA | NA | NA |
| | 68111168 | | | | |
| ARFIP1 | chr4: 153831372- | NA | NA | NA | NA |
| | 153833063 | | | | |
| ARFIP2 | NA | NA | NA | NA | NA |
| ARG1 | NA | 861.219 | −14.338252 | uc003qco.2 | chr6: 131904948- |
| | | | | | 131905465 |
| ARGFX | NA | 1619.53 | −11.933535 | uc003eef.3 | chr3: 121305448- |
| | | | | | 121309469 |
| ARHGAP10 | NA | NA | NA | NA | NA |
| ARHGAP11A | chr15: 32928140- | NA | NA | NA | NA |
| | 32928382 | | | | |
| ARHGAP18 | chr6: 129898240- | NA | NA | NA | NA |
| | 129899657 | | | | |
| ARHGAP21 | NA | NA | NA | NA | NA |
| ARHGAP29 | chr1: 94634463- | 1498.07 | −3.625596 | uc001dqj.4 | chr1: 94634463- |
| | 94639424 | | | | 94639424 |
| ARHGAP32 | NA | 639.843 | −7.604816 | uc009zco.3 | chr11: 128834955- |
| | | | | | 128838801 |
| ARHGAP33 | chr19: 36279332- | NA | NA | NA | NA |
| | 36279724 | | | | |
| ARHGAP42 | NA | NA | NA | NA | NA |
| ARHGAP6 | NA | NA | NA | NA | NA |
| ARHGEF12 | NA | 709.837 | −5.142271 | uc001pxl.2 | chr11: 120355787- |
| | | | | | 120360645 |
| ARHGEF15 | chr1: 8216063- | NA | NA | NA | NA |
| | 8217349 | | | | |
| ARHGEF16 | chr1: 3390213- | NA | NA | NA | NA |
| | 3391021 | | | | |
| ARHGEF28 | chr5: 73236839- | NA | NA | NA | NA |
| | 73237818 | | | | |
| ARHGEF3 | NA | 307.134 | −5.264453 | uc011bev.1 | chr3: 56761446- |
| | | | | | 56763297 |
| ARHGEF35 | chr7: 143883176- | 1025.27 | −8.23548 | uc003wdz.2 | chr7: 143883176- |
| | 143884021 | | | | 143884021 |
| ARHGEF38 | chr4: 106552207- | NA | NA | NA | NA |
| | 106552837 | | | | |
| ARHGEF4 | NA | 1217.03 | −5.511759 | uc002trz.1 | chr2: 131704259- |
| | | | | | 131704803 |
| ARID1B | NA | 1163.4 | −11.711323 | uc003qqq.1 | chr6: 157470147- |
| | | | | | 157473538 |
| ARID2 | chr12: 46233285- | NA | NA | NA | NA |
| | 46236017 | | | | |
| ARID5A | NA | 243.483 | −4.748682 | uc002we.3 | chr2: 97218051- |
| | | | | | 97218371 |
| ARL13B | NA | 459.796 | −5.490174 | uc003drd.3 | chr3: 93772108- |
| | | | | | 93774522 |
| ARL14EPL | chr5: 115394645- | 1099.32 | −9.973846 | uc021yct.1 | chr5: 115394645- |
| | 115394827 | | | | 115394827 |
| ARL15 | NA | 1354.14 | −12.232902 | uc003jpg.1 | chr5: 53180614- |
| | | | | | 53182390 |
| ARL17A | chr17: 44376500- | NA | NA | NA | NA |
| | 44376811 | | | | |
| ARL4A | chr7: 12728483- | NA | NA | NA | NA |
| | 12730558 | | | | |
| ARL5B | NA | NA | NA | NA | NA |
| ARL6IP1 | chr16: 18802991- | NA | NA | NA | NA |
| | 18804573 | | | | |
| ARL6IP6 | chr2: 153616355- | NA | NA | NA | NA |
| | 153617767 | | | | |
| ARL8B | chr3: 5220399- | 278.075 | −3.471118 | uc003bqg.3 | chr3: 5220399- |
| | 5222601 | | | | 5222601 |
| ARMCX5 | chrX: 101858747- | NA | NA | NA | NA |
| | 101859085 | | | | |
| ARNTL2 | NA | 1157.58 | −8.660753 | uc001mko.3 | chr11: 13408304- |
| | | | | | 13408812 |
| ARNTL2 | NA | NA | NA | NA | NA |
| ARPC3 | NA | NA | NA | NA | NA |
| ARRDC4 | chr15: 98514418- | NA | NA | NA | NA |
| | 98517068 | | | | |
| ARRDC5 | NA | NA | NA | NA | NA |
| ARSA | NA | NA | NA | NA | NA |
| ARSB | NA | NA | NA | NA | NA |
| ARSE | NA | 769.679 | −9.56035 | uc004crc.4 | chrX: 2852673- |
| | | | | | 2852872 |
| ART4 | chr12: 14982245- | 773.985 | −8.083501 | uc001rcl.1 | chr12: 14982245- |
| | 14982304 | | | | 14982304 |
| ARV1 | chr1: 231133010- | 507.526 | −5.849707 | uc001huh.3 | chr1: 231135884- |
| | 231133147 | | | | 231136479 |
| ASAP1 | chr8: 131064351- | NA | NA | NA | NA |
| | 131066976 | | | | |
| ASB1 | NA | NA | NA | NA | NA |
| ASB11 | NA | 316.166 | −6.42245 | uc004cwo.2 | chrX: 15299831- |
| | | | | | 15301626 |
| ASB16 | chr17: 42255759- | NA | NA | NA | NA |
| | 42256451 | | | | |
| ASB17 | NA | 762.986 | −8.695003 | uc001dhe.2 | chr1: 76384558- |
| | | | | | 76384636 |
| ASB7 | chr15: 101170256- | NA | NA | NA | NA |
| | 101170414 | | | | |
| ASMTL | NA | NA | NA | NA | NA |
| ASNSD1 | NA | NA | NA | NA | NA |
| ASPA | NA | NA | NA | NA | NA |
| ASPDH | chr19: 51014857- | NA | NA | NA | NA |
| | 51014986 | | | | |
| ASPH | chr8: 62413115- | NA | NA | NA | NA |
| | 62415917 | | | | |
| ASPM | NA | NA | NA | NA | NA |
| ASS1 | NA | 700.494 | −11.03023 | uc004bzm.3 | chr9: 133376409- |
| | | | | | 133376661 |
| ASTE1 | NA | NA | NA | NA | NA |
| ASTN1 | chr1: 176826441- | NA | NA | NA | NA |
| | 176826746 | | | | |
| ASTN2 | chr9: 119187504- | NA | NA | NA | NA |
| | 119188129 | | | | |
| ASXL1 | NA | 1044.05 | −13.683314 | uc002wxt.3 | chr20: 30959973- |
| | | | | | 30960352 |
| ASZ1 | NA | 867.616 | −7.877995 | uc003vjb.2 | chr7: 117003276- |
| | | | | | 117003649 |
| ATAD2 | chr8: 124332091- | 625.6 | −12.722897 | uc011lii.2 | chr8: 124332091- |
| | 124333373 | | | | 124333373 |
| ATAD5 | NA | NA | NA | NA | NA |
| ATE1 | NA | 532.504 | −9.827832 | uc001lfp.3 | chr10: 123502625- |
| | | | | | 123503194 |
| ATF1 | NA | NA | NA | NA | NA |
| ATF3 | NA | 473.6 | −5.034036 | uc001hjf.3 | chr1: 212792898- |
| | | | | | 212794119 |
| ATF7IP | chr12: 14628959- | 1237.47 | −5.440922 | uc001rbu.3 | chr12: 14628959- |
| | 14629347 | | | | 14629347 |
| ATF7IP2 | chr16: 10576107- | NA | NA | NA | NA |
| | 10577495 | | | | |
| ATG101 | chr12: 52470975- | NA | NA | NA | NA |
| | 52471279 | | | | |
| ATG12 | chr5: 115163894- | NA | NA | NA | NA |
| | 115167500 | | | | |
| ATG14 | chr14: 55833109- | NA | NA | NA | NA |
| | 55836336 | | | | |
| ATG2B | NA | NA | NA | NA | NA |
| ATG4D | NA | 759.658 | −7.554543 | uc002mov.3 | chr19: 10663744- |
| | | | | | 10664094 |
| ATIC | NA | 610.193 | −6.569674 | uc002vex.4 | chr2: 216214379- |
| | | | | | 216214496 |
| ATL2 | chr2: 38522029- | 698.796 | −5.609656 | uc010ynm.2 | chr2: 38522029- |
| | 38523624 | | | | 38523624 |
| ATM | NA | 781.042 | −2.846765 | uc001pkd.4 | chr11: 108160533- |
| | | | | | 108161367 |
| ATOH8 | chr2: 86000032- | NA | NA | NA | NA |
| | 86002336 | | | | |
| ATP10A | chr15: 25923860- | 481.682 | −6.025016 | uc010ayu.3 | chr15: 25923860- |
| | 25924487 | | | | 25924487 |
| ATP11C | chrX: 138808505- | NA | NA | NA | NA |
| | 138811121 | | | | |
| ATP13A3 | chr3: 194123403- | NA | NA | NA | NA |
| | 194126647 | | | | |
| ATP13A4 | chr3: 193119866- | NA | NA | NA | NA |
| | 193120440 | | | | |
| ATP1A2 | chr1: 160106826- | NA | NA | NA | NA |
| | 160106992 | | | | |
| ATP1B2 | chr17: 7559214- | NA | NA | NA | NA |
| | 7561089 | | | | |
| ATP1B3 | chr3: 141644544- | NA | NA | NA | NA |
| | 141645382 | | | | |
| ATP1B4 | chrX: 119513490- | 963.792 | −7.108402 | uc004esq.3 | chrX: 119513490- |
| | 119517104 | | | | 119517104 |
| ATP2B2 | chr3: 10429568- | 716.295 | −6.832539 | uc010hdp.2 | chr3: 10429568- |
| | 10429919 | | | | 10429919 |
| ATP2B4 | chr1: 203708983- | 1832.15 | −13.611216 | uc001gzv.3 | chr1: 203708700- |
| | 203713209 | | | | 203713209 |
| ATP2C2 | chr16: 84497339- | NA | NA | NA | NA |
| | 84497793 | | | | |
| ATP5A1 | chr18: 43664110- | NA | NA | NA | NA |
| | 43664247 | | | | |
| ATP5B | NA | NA | NA | NA | NA |
| ATP5C1 | chr10: 7849626- | 762.346 | −6.033502 | uc001iju.3 | chr10: 7849622- |
| | 7849762 | | | | 7849762 |
| ATP5G1 | NA | 360.958 | −5.826945 | uc002iog.3 | chr17: 46973132- |
| | | | | | 46973232 |
| ATP5G2 | chr12: 54058944- | NA | NA | NA | NA |
| | 54059097 | | | | |
| ATP5G3 | chr2: 176040986- | NA | NA | NA | NA |
| | 176043015 | | | | |
| ATP5J2 | chr7: 99055784- | NA | NA | NA | NA |
| | 99055949 | | | | |
| ATP6AP1 | NA | NA | NA | NA | NA |
| ATP6V0A2 | NA | NA | NA | NA | NA |
| ATP6V0B | chr1: 44443084- | 401.476 | −4.37522 | uc001cld.3 | chr1: 44443680- |
| | 44443967 | | | | 44443972 |
| ATP6V0D1 | NA | 1493.42 | −17.594665 | uc010vjn.1 | chr16: 67471917- |
| | | | | | 67472430 |
| ATP6V0E2 | chr7: 149576767- | NA | NA | NA | NA |
| | 149577787 | | | | |
| ATP6V1A | NA | 1372.17 | −14.425225 | uc003eao.3 | chr3: 113528275- |
| | | | | | 113530905 |
| ATP6V1B2 | chr8: 20077914- | 958.921 | −11.537631 | uc003wzp.3 | chr8: 20077914- |
| | 20079207 | | | | 20079207 |
| ATP6V1C1 | NA | 994.969 | −7.72899 | uc003ykz.4 | chr8: 104080976- |
| | | | | | 104085285 |
| ATP6V1C2 | chr2: 10923390- | NA | NA | NA | NA |
| | 10925236 | | | | |
| ATP6V1D | chr14: 67804581- | NA | NA | NA | NA |
| | 67805337 | | | | |
| ATP6V1E1 | NA | 326.487 | −6.120118 | uc002zmr.1 | chr22: 18074903- |
| | | | | | 18075439 |
| ATP6V1F | chr7: 128505633- | NA | NA | NA | NA |
| | 128505903 | | | | |
| ATP6V1G1 | NA | NA | NA | NA | NA |
| ATP6V1G3 | chr1: 198492352- | 1129.38 | −2.751467 | uc009wzd.3 | chr1: 198492352- |
| | 198492520 | | | | 198492520 |
| ATP7A | chrX: 77264911- | 698.288 | −3.097801 | uc004ecw.2 | chrX: 77264911- |
| | 77268087 | | | | 77268087 |
| ATP8B1 | chr18: 55313659- | NA | NA | NA | NA |
| | 55315719 | | | | |
| ATP8B2 | chr1: 154310151- | 1016.17 | −5.237598 | uc001few.3 | chr1: 154310151- |
| | 154310322 | | | | 154310322 |
| ATP8B3 | chr19: 1798760- | NA | NA | NA | NA |
| | 1799649 | | | | |
| ATP8B4 | NA | NA | NA | NA | NA |
| ATR | NA | 417.49 | −7.543463 | uc003euy.1 | chr3: 142168080- |
| | | | | | 142171897 |
| ATRNL1 | chr10: 117704291- | 523.868 | −8.90812 | uc001lcg.3 | chr10: 117704291- |
| | 117708496 | | | | 117708496 |
| ATXN1 | NA | NA | NA | NA | NA |
| ATXN2 | NA | 852.962 | −7.708961 | uc001tsh.3 | chr12: 111890018- |
| | | | | | 111890615 |
| ATXN7 | chr3: 63982178- | 1040.81 | −4.127988 | uc003dlw.4 | chr3: 63985144- |
| | 63982295 | | | | 63989136 |
| ATXN7L2 | chr1: 110035223- | NA | NA | NA | NA |
| | 110035420 | | | | |
| AUP1 | chr2: 74754068- | 1651.08 | −11.321314 | uc002sme.3 | chr2: 74753775- |
| | 74754203 | | | | 74753926 |
| AURKB | chr17: 8108049- | NA | NA | NA | NA |
| | 8108188 | | | | |
| AURKC | chr19: 57746786- | 810.654 | −7.756988 | uc002qoc.3 | chr19: 57746786- |
| | 57746916 | | | | 57746916 |
| AVIL | NA | 273.732 | −2.918401 | uc001sqj.2 | chr12: 58191160- |
| | | | | | 58191664 |
| AZI2 | NA | 486.004 | −7.766873 | uc003cee.4 | chr3: 28367995- |
| | | | | | 28368293 |
| B2M | chr15: 45009805- | NA | NA | NA | NA |
| | 45010357 | | | | |
| B3GALT1 | chr2: 168726531- | NA | NA | NA | NA |
| | 168727366 | | | | |
| B3GALT4 | NA | 631.528 | −6.642339 | uc003odr.3 | chr6: 33246334- |
| | | | | | 33246602 |
| B3GNT4 | NA | 497.574 | −5.924138 | uc001ubx.3 | chr12: 122691936- |
| | | | | | 122692084 |
| B4GALNT2 | NA | NA | NA | NA | NA |
| B4GALT4 | NA | 917.872 | −5.847207 | uc003ece.1 | chr3: 118930587- |
| | | | | | 118935082 |
| B4GALT6 | NA | NA | NA | NA | NA |
| BABAM1 | chr19: 17389858- | NA | NA | NA | NA |
| | 17390162 | | | | |
| BAGE3 | chr21: 11026723- | 526.583 | −6.212389 | uc002yit.1 | chr21: 11020813- |
| | 11026839 | | | | 11020917 |
| BARD1 | NA | NA | NA | NA | NA |
| BAX | chr19: 49464894- | NA | NA | NA | NA |
| | 49465055 | | | | |
| BAZ1B | NA | NA | NA | NA | NA |
| BAZ2A | NA | 695.084 | −7.711833 | uc001slo.1 | chr12: 56989380- |
| | | | | | 56992401 |
| BAZ2B | NA | 720.511 | −3.126428 | uc002uar.1 | chr2: 160260901- |
| | | | | | 160261329 |
| BBS4 | chr15: 73029929- | NA | NA | NA | NA |
| | 73030817 | | | | |
| BBS5 | NA | 823.529 | −10.7821 | uc002uet.3 | chr2: 170361093- |
| | | | | | 170363165 |
| BBS9 | chr7: 33353924- | NA | NA | NA | NA |
| | 33354183 | | | | |
| BCAP29 | chr7: 107258809- | NA | NA | NA | NA |
| | 107263762 | | | | |
| BCAR3 | chr1: 94036889- | 1290.56 | −9.593388 | uc009wdm.1 | chr1: 94036889- |
| | 94037097 | | | | 94037097 |
| BCAR4 | chr16: 11913692- | NA | NA | NA | NA |
| | 11913776 | | | | |
| BCAS4 | NA | 1237.53 | −13.639567 | uc002xvq.3 | chr20: 49493073- |
| | | | | | 49493714 |
| BCCIP | chr10: 127524844- | 542.377 | −4.232466 | uc021qar.1 | chr10: 127522543- |
| | 127525124 | | | | 127523219 |
| BCL10 | NA | 568.04 | −5.636385 | uc021opd.1 | chr1: 85731460- |
| | | | | | 85733309 |
| BCL11A | NA | 699.15 | −9.206331 | uc002sab.3 | chr2: 60678302- |
| | | | | | 60679709 |
| BCL2 | NA | NA | NA | NA | NA |
| BCL2A1 | NA | 224.821 | −4.888625 | uc002bfc.4 | chr15: 80253232- |
| | | | | | 80253408 |
| BCL2L11 | chr2: 111921809- | 1163.32 | −4.345747 | uc002tgt.1 | chr2: 111886329- |
| | 111926022 | | | | 111886423 |
| BCL2L12 | NA | NA | NA | NA | NA |
| BCL2L14 | chr12: 12247526- | 499.996 | −5.844576 | uc001rac.3 | chr12: 12251923- |
| | 12247864 | | | | 12252627 |
| BCL2L15 | chr1: 114419436- | NA | NA | NA | NA |
| | 114423744 | | | | |
| BCL7B | chr7: 72950683- | NA | NA | NA | NA |
| | 72951627 | | | | |
| BCL9 | chr1: 147096761- | NA | NA | NA | NA |
| | 147098015 | | | | |
| BCLAF1 | chr6: 136578001- | NA | NA | NA | NA |
| | 136582247 | | | | |
| BCO2 | NA | NA | NA | NA | NA |
| BCOR | NA | NA | NA | NA | NA |
| BCORL1 | NA | NA | NA | NA | NA |
| BCORP1 | chrY: 21628342- | 276.128 | −4.912083 | uc011nav.1 | chrY: 21628342- |
| | 21628913 | | | | 21628913 |
| BCR | NA | 802.894 | −5.02011 | uc002zww.3 | chr22: 23657710- |
| | | | | | 23660224 |
| BDH1 | NA | 955.054 | −12.226262 | uc003fxr.3 | chr3: 197236654- |
| | | | | | 197238765 |
| BDH2 | NA | 712.546 | −7.80563 | uc003hwz.3 | chr4: 103998782- |
| | | | | | 104000858 |
| BDKRB2 | chr14: 96704154- | 432.142 | −3.854518 | uc010avl.2 | chr14: 96704154- |
| | 96710503 | | | | 96710503 |
| BDNF | chr11: 27676442- | NA | NA | NA | NA |
| | 27679367 | | | | |
| BDP1 | chr5: 70807039- | 1390.92 | −9.777427 | uc003kbp.1 | chr5: 70860713- |
| | 70807178 | | | | 70863649 |
| BEND2 | chrX: 18181051- | 683.399 | −7.950195 | uc004cyj.4 | chrX: 18181051- |
| | 18183128 | | | | 18183128 |
| BEND7 | chr10: 13480484- | 515.959 | −3.321475 | uc001imm.2 | chr10: 13480484- |
| | 13481171 | | | | 13481171 |
| BET1 | NA | 307.239 | −6.987781 | uc003unf.1 | chr7: 93621000- |
| | | | | | 93623541 |
| BEX2 | NA | NA | NA | NA | NA |
| BEX4 | NA | NA | NA | NA | NA |
| BEX5 | NA | NA | NA | NA | NA |
| BHLHB9 | NA | NA | NA | NA | NA |
| BHLHE22 | NA | 486.785 | −6.379406 | uc003xvi.3 | chr8: 65494494- |
| | | | | | 65496191 |
| BHMT | NA | 256.743 | −7.305491 | uc003kfu.4 | chr5: 78426940- |
| | | | | | 78428113 |
| BHMT2 | NA | NA | NA | NA | NA |
| BICD1 | NA | 370.386 | −7.126125 | uc001rku.3 | chr12: 32530562- |
| | | | | | 32531141 |
| BIK | chr22: 43525312- | NA | NA | NA | NA |
| | 43525718 | | | | |
| BIN3 | NA | 698.134 | −5.785125 | uc003xcl.3 | chr8: 22477947- |
| | | | | | 22478934 |
| BIRG5 | chr17: 76210933- | NA | NA | NA | NA |
| | 76211066 | | | | |
| BIRC7 | chr20: 61871608- | NA | NA | NA | NA |
| | 61871854 | | | | |
| BIRC8 | chr19: 53792854- | NA | NA | NA | NA |
| | 53792916 | | | | |
| BIVM | NA | 715.127 | −11.941018 | uc001vps.3 | chr13: 103492216- |
| | | | | | 103493888 |
| BLM | NA | NA | NA | NA | NA |
| BLOC1S6 | chr15: 45898713- | NA | NA | NA | NA |
| | 45901909 | | | | |
| BLZF1 | chr1: 169356421- | 806.212 | −5.764549 | uc001gfw.3 | chr1: 169346275- |
| | 169357539 | | | | 169346621 |
| BMP10 | NA | 725.168 | −9.439893 | uc002sez.1 | chr2: 69092613- |
| | | | | | 69092762 |
| BMP2K | chr4: 79800084- | NA | NA | NA | NA |
| | 79800594 | | | | |
| BMP3 | chr4: 81974691- | NA | NA | NA | NA |
| | 81978685 | | | | |
| BMP8B | chr1: 40223903- | NA | NA | NA | NA |
| | 40226090 | | | | |
| BMPR1A | NA | NA | NA | NA | NA |
| BMPR1B | chr4: 96075825- | NA | NA | NA | NA |
| | 96079601 | | | | |
| BMPR2 | chr2: 203424670- | NA | NA | NA | NA |
| | 203432474 | | | | |
| BNIP2 | NA | NA | NA | NA | NA |
| BNIP3 | NA | 892.771 | −5.227471 | uc001lkv.1 | chr10: 133781204- |
| | | | | | 133782027 |
| BOC | chr3: 113005710- | NA | NA | NA | NA |
| | 113006305 | | | | |
| BOLA3 | chr2: 74362528- | 475.71 | −9.198202 | uc002skc.1 | chr2: 74362528- |
| | 74362648 | | | | 74362719 |
| BPHL | chr6: 3152810- | NA | NA | NA | NA |
| | 3153432 | | | | |
| BPY2B | NA | NA | NA | NA | NA |
| BRD1 | chr22: 50166937- | 1375.83 | −13.524208 | uc011arf.2 | chr22: 50166937- |
| | 50167880 | | | | 50167880 |
| BRD3 | NA | 1718.57 | −2.327148 | uc004cew.3 | chr9: 136895446- |
| | | | | | 136898711 |
| BRD7 | chr16: 50352929- | NA | NA | NA | NA |
| | 50353121 | | | | |
| BRD9 | chr5: 863850- | 1056.92 | −16.080428 | uc003jbl.3 | chr5: 863850- |
| | 864582 | | | | 864582 |
| BRINP1 | NA | NA | NA | NA | NA |
| BRINP3 | chr1: 190066797- | NA | NA | NA | NA |
| | 190067147 | | | | |
| BRIX1 | NA | 398.02 | −14.91091 | uc003jiz.3 | chr5: 34920034- |
| | | | | | 34922014 |
| BRWD1 | NA | NA | NA | NA | NA |
| BSPRY | chr9: 116132423- | NA | NA | NA | NA |
| | 116133513 | | | | |
| BST1 | chr4: 15733469- | 1399.18 | −8.522056 | uc003goh.4 | chr4: 15733469- |
| | 15733796 | | | | 15733796 |
| BTAF1 | NA | NA | NA | NA | NA |
| BTBD10 | NA | 1411.48 | −7.305804 | uc010rcl.2 | chr11: 13409556- |
| | | | | | 13410377 |
| BTBD17 | NA | NA | NA | NA | NA |
| BTBD7 | chr14: 93721866- | NA | NA | NA | NA |
| | 93723507 | | | | |
| BTD | chr3: 15686996- | NA | NA | NA | NA |
| | 15687325 | | | | |
| BTF3 | NA | NA | NA | NA | NA |
| BTG2 | NA | NA | NA | NA | NA |
| BTN2A2 | chr6: 26394558- | NA | NA | NA | NA |
| | 26395100 | | | | |
| BTN3A3 | NA | 419.415 | −8.815144 | uc003nhz.3 | chr6: 26452640- |
| | | | | | 26453643 |
| BUB1 | NA | NA | NA | NA | NA |
| BUB1B | NA | NA | NA | NA | NA |
| BUB3 | NA | 890.287 | −5.076864 | uc001lhf.4 | chr10: 124922349- |
| | | | | | 124924881 |
| BUD13 | NA | NA | NA | NA | NA |
| BZW2 | chr7: 16725731- | 566.803 | −6.877934 | uc003stj.2 | chr7: 16745714- |
| | 16727179 | | | | 16746148 |
| C10orf111 | NA | 1325.26 | −10.820286 | uc001inw.3 | chr10: 15137384- |
| | | | | | 15138355 |
| C10orf131 | NA | NA | NA | NA | NA |
| C10orf32 | chr10: 104623487- | NA | NA | NA | NA |
| | 104624718 | | | | |
| C10orf53 | chr10: 50902649- | NA | NA | NA | NA |
| | 50902948 | | | | |
| C10orf82 | NA | 1189.36 | −8.80932 | uc001lcr.3 | chr1: 118423207- |
| | | | | | 118423680 |
| C10orf88 | NA | 630.663 | −7.755551 | uc001lgw.2 | chr10: 124690419- |
| | | | | | 124691942 |
| C11orf24 | NA | NA | NA | NA | NA |
| C11orf57 | NA | 744.957 | −8.506944 | uc001pmr.4 | chr11: 111953697- |
| | | | | | 111955874 |
| C11orf58 | NA | 437.873 | −11.470382 | uc001mmk.2 | chr11: 16776652- |
| | | | | | 16779901 |
| C11orf68 | chr11: 65684283- | NA | NA | NA | NA |
| | 65684929 | | | | |
| C11orf70 | chr11: 101946636- | 979.126 | −13.928117 | uc001pgo.3 | chr11: 101946636- |
| | 101946842 | | | | 101946842 |
| C11orf88 | chr11: 111405194- | 704.527 | −4.598357 | uc001pln.4 | chr11: 111407553- |
| | 111407755 | | | | 111407756 |
| C12orf29 | chr12: 88442200- | NA | NA | NA | NA |
| | 88443937 | | | | |
| C12orf50 | chr12: 88377174- | NA | NA | NA | NA |
| | 88379609 | | | | |
| C12orf60 | chr12: 14976608- | NA | NA | NA | NA |
| | 14976791 | | | | |
| C12orf65 | NA | 371.873 | −4.531871 | uc001uen.3 | chr12: 123741579- |
| | | | | | 123742506 |
| C12orf73 | chr12: 104343981- | NA | NA | NA | NA |
| | 104345300 | | | | |
| C14orf1 | chr14: 76117233- | NA | NA | NA | NA |
| | 76117897 | | | | |
| C14orf105 | NA | 202.583 | −6.325793 | uc001xcy.2 | chr14: 57936595- |
| | | | | | 57938072 |
| C14orf159 | NA | 908.756 | −7.399268 | uc001xyv.2 | chr14: 91691178- |
| | | | | | 91691703 |
| C14orf2 | NA | 862.641 | −7.171605 | uc001yoi.4 | chr14: 104378625- |
| | | | | | 104379002 |
| C14orf28 | chr14: 45374754- | NA | NA | NA | NA |
| | 45376460 | | | | |
| C14orf37 | chr14: 58470808- | 221.268 | −3.711218 | uc001xde.3 | chr14: 58582866- |
| | 58471453 | | | | 58583043 |
| C14orf39 | chr14: 60902674- | NA | NA | NA | NA |
| | 60903562 | | | | |
| C15orf32 | chr15: 93043631- | NA | NA | NA | NA |
| | 93044347 | | | | |
| C15orf40 | chr15: 83673145- | 216.3 | −4.126155 | uc010uon.2 | chr15: 83657715- |
| | 83674348 | | | | 83657825 |
| C15orf57 | NA | NA | NA | NA | NA |
| C15orf59 | chr15: 74032141- | NA | NA | NA | NA |
| | 74032257 | | | | |
| C15orf61 | NA | 1041.24 | −8.414177 | uc002aqs.3 | chr15: 67818884- |
| | | | | | 67819641 |
| C15orf65 | chr15: 55710769- | NA | NA | NA | NA |
| | 55710910 | | | | |
| C16orf59 | NA | NA | NA | NA | NA |
| C16orf62 | chr16: 19628263- | NA | NA | NA | NA |
| | 19628490 | | | | |
| C16orf74 | chr16: 85741124- | NA | NA | NA | NA |
| | 85741613 | | | | |
| C16orf93 | NA | NA | NA | NA | NA |
| C17orf104 | NA | 309.363 | −5.20203 | uc002igz.3 | chr17: 42746907- |
| | | | | | 42748590 |
| C17orf96 | chr17: 36827959- | NA | NA | NA | NA |
| | 36829608 | | | | |
| C18orf42 | NA | 1486.47 | −12.29373 | uc010wzc.1 | chr18: 5143672- |
| | | | | | 5145560 |
| C18orf63 | chr18: 72023716- | NA | NA | NA | NA |
| | 72026422 | | | | |
| C19orf44 | NA | 388.018 | −5.671791 | uc002neg.3 | chr19: 16628813- |
| | | | | | 16629430 |
| C19orf66 | chr19: 10198103- | 216.777 | −5.661259 | uc002mmt.2 | chr19: 10198103- |
| | 10198936 | | | | 10198936 |
| C1D | chr2: 68269332- | NA | NA | NA | NA |
| | 68270020 | | | | |
| C1GALT1 | chr7: 7274197- | NA | NA | NA | NA |
| | 7274928 | | | | |
| C1QL3 | chr10: 16555742- | NA | NA | NA | NA |
| | 16556526 | | | | |
| C1orf101 | chr1: 244803230- | 812.542 | −13.112159 | uc001iak.1 | chr1: 244773674- |
| | 244803662 | | | | 244773890 |
| C1orf112 | chr1: 169821195- | 426.765 | −13.267141 | uc001ggo.3 | chr1: 169773390- |
| | 169821378 | | | | 169773526 |
| C1orf131 | chr1: 231359509- | NA | NA | NA | NA |
| | 231360024 | | | | |
| C1orf140 | NA | NA | NA | NA | NA |
| C1orf216 | chr1: 36179477- | 362.989 | −9.283392 | uc001bzh.1 | chr1: 36179477- |
| | 36181232 | | | | 36181232 |
| C1orf27 | NA | 1145.56 | −9.31031 | uc021pgg.1 | chr1: 186388141- |
| | | | | | 186390503 |
| C1orf35 | chr1: 228288428- | NA | NA | NA | NA |
| | 228288831 | | | | |
| C1orf53 | NA | NA | NA | NA | NA |
| C20orf173 | NA | 411.666 | −7.879615 | uc002xcp.2 | chr20: 34114799- |
| | | | | | 34115078 |
| C21orf59 | chr21: 33973984- | NA | NA | NA | NA |
| | 33974172 | | | | |
| C22orf39 | NA | 941.947 | −5.498829 | uc002zpk.2 | chr22: 19430673- |
| | | | | | 19431787 |
| C2CD2 | chr21: 43321062- | NA | NA | NA | NA |
| | 43321658 | | | | |
| C2CD3 | NA | NA | NA | NA | NA |
| C2CD4A | NA | 570.55 | −9.63106 | uc002ahf.4 | chr15: 62360923- |
| | | | | | 62363116 |
| C2CD4B | NA | 479.309 | −4.159832 | uc002ahg.2 | chr15: 62455737- |
| | | | | | 62456088 |
| C2CD5 | NA | 1289.04 | −10.117767 | uc009ziy.1 | chr12: 2263O962- |
| | | | | | 22631186 |
| C2orf42 | NA | 796.429 | −9.828334 | uc002sgh.3 | chr2: 70377017- |
| | | | | | 70377487 |
| C2orf43 | NA | 568.202 | −6.53487 | uc002rec.3 | chr2: 20884818- |
| | | | | | 20886662 |
| C2orf47 | chr2: 200828535- | NA | NA | NA | NA |
| | 200828847 | | | | |
| C2orf49 | NA | 1702.25 | −19.684259 | uc002tcs.1 | chr2: 105961829- |
| | | | | | 105961984 |
| C2orf57 | chr2: 232458851- | NA | NA | NA | NA |
| | 232458994 | | | | |
| C2orf72 | chr2: 231911954- | 412.346 | −5.466334 | uc010zme.2 | chr2: 231911954- |
| | 231914427 | | | | 231914427 |
| C2orf78 | NA | NA | NA | NA | NA |
| C2orf80 | NA | NA | NA | NA | NA |
| C3orf22 | chr3: 126268519- | NA | NA | NA | NA |
| | 126268710 | | | | |
| C3orf30 | NA | 673 893 | −9.241189 | uc003ecb.1 | chr3: 118870140- |
| | | | | | 118870302 |
| C3orf33 | chr3: 155480401- | NA | NA | NA | NA |
| | 155481305 | | | | |
| C3orf36 | chr3: 133646990- | NA | NA | NA | NA |
| | 133647149 | | | | |
| C3orf58 | NA | NA | NA | NA | NA |
| C3orf70 | chr3: 184795838- | 1005.46 | −11.979047 | uc003fpd.3 | chr3: 184795838- |
| | 184800794 | | | | 184800794 |
| C4BPA | chr1: 207318063- | NA | NA | NA | NA |
| | 207318317 | | | | |
| C4BPB | NA | NA | NA | NA | NA |
| C4orf17 | chr4: 100463267- | NA | NA | NA | NA |
| | 100463460 | | | | |
| C4orf26 | chr4: 76489650- | NA | NA | NA | NA |
| | 76491103 | | | | |
| C4orf27 | NA | 210.255 | −6.369736 | uc003isl.4 | chr4: 170650619- |
| | | | | | 170650728 |
| C4orf29 | chr4: 128951832- | 1134.53 | −7.232481 | uc003ift.3 | chr4: 128956969- |
| | 128952455 | | | | 128957476 |
| C5 | chr9: 123714614- | NA | NA | NA | NA |
| | 123715016 | | | | |
| C5AR1 | NA | 1203.45 | −7.54251 | uc002pgj.1 | chr19: 47824088- |
| | | | | | 47825327 |
| C5orf22 | NA | NA | NA | NA | NA |
| C5orf46 | NA | 409.746 | −2.463336 | uc010jgp.3 | chr5: 147269579- |
| | | | | | 147269787 |
| C5orf56 | NA | 699.638 | −7.933695 | uc003kwz.2 | chr5: 131811625- |
| | | | | | 131811736 |
| C5orf58 | chr5: 169673118- | NA | NA | NA | NA |
| | 169673235 | | | | |
| C6orf1 | chr6: 34214157- | NA | NA | NA | NA |
| | 34214290 | | | | |
| C6orf132 | chr6: 42068857- | NA | NA | NA | NA |
| | 42071498 | | | | |
| C6orf201 | chr6: 4125218- | NA | NA | NA | NA |
| | 4126353 | | | | |
| C6orf226 | chr6: 42858003- | NA | NA | NA | NA |
| | 42858220 | | | | |
| C6orf62 | chr6: 24705090- | NA | NA | NA | NA |
| | 24706364 | | | | |
| C7orf31 | NA | 551.093 | −6.453775 | uc003sxn.1 | chr7: 25174316- |
| | | | | | 25175590 |
| C7orf34 | NA | 448.101 | −6.867483 | uc003wca.2 | chr7: 142637675- |
| | | | | | 142637957 |
| C7orf62 | chr7: 88423420- | NA | NA | NA | NA |
| | 88423494 | | | | |
| C7orf73 | NA | NA | NA | NA | NA |
| C8orf34 | NA | NA | NA | NA | NA |
| C8orf48 | NA | 595.848 | −10.44353 | uc003wwp.3 | chr8: 13425461- |
| | | | | | 13425797 |
| C9orf131 | NA | 370.403 | −5.746329 | uc003zvu.3 | chr9: 35045867- |
| | | | | | 35045988 |
| C9orf139 | NA | 628.628 | −8.43788 | uc004ckp.1 | chr9: 139929507- |
| | | | | | 139931234 |
| C9orf156 | chr9: 100673200- | 1218.44 | −4.147975 | uc004axv.1 | chr9: 100666772- |
| | 100675183 | | | | 100667014 |
| C9orf24 | NA | 213.407 | −4.268978 | uc003zuf.1 | chr9: 34379017- |
| | | | | | 34379073 |
| C9orf3 | chr9: 97694037- | 442.075 | −8.233932 | uc004aux.2 | chr9: 97694037- |
| | 97695955 | | | | 97695955 |
| C9orf47 | chr9: 91606904- | NA | NA | NA | NA |
| | 91611057 | | | | |
| C9orf64 | chr9: 86553227- | NA | NA | NA | NA |
| | 86554425 | | | | |
| C9orf84 | chr9: 114448901- | 562.091 | −0.300984 | uc004bfq.3 | chr9: 114448901- |
| | 114449049 | | | | 114449049 |
| C9orf91 | chr9: 117405594- | NA | NA | NA | NA |
| | 117408703 | | | | |
| CA2 | NA | NA | NA | NA | NA |
| CA3 | NA | NA | NA | NA | NA |
| CA5B | chrX: 15800788- | 612.695 | −4.999425 | uc004cxe.3 | chrX: 15800788- |
| | 15805748 | | | | 15805748 |
| CAB39L | chr13: 49882786- | 451.099 | −6.36467 | uc001vcw.3 | chr13: 49882786- |
| | 49884949 | | | | 49884949 |
| CABLES1 | chr18: 20837332- | 1220.06 | −3.867449 | uc002kub.2 | chr18: 20837332- |
| | 20840434 | | | | 20840434 |
| CABS1 | NA | NA | NA | NA | NA |
| CACNB2 | chr10: 18828654- | 1047.67 | −3.9971 | uc001ipr.2 | chr10: 18828654- |
| | 18830688 | | | | 18830688 |
| CADM1 | chr11: 115039951- | NA | NA | NA | NA |
| | 115040308 | | | | |
| CALCA | NA | 891.812 | −5.036312 | uc001mlt.2 | chr11: 14988215- |
| | | | | | 14988647 |
| CALCB | NA | NA | NA | NA | NA |
| CALD1 | NA | 251.334 | −10.429201 | uc003vrz.3 | chr7: 134653097- |
| | | | | | 134655480 |
| CALHM2 | NA | NA | NA | NA | NA |
| CALM2 | chr2: 47387221- | NA | NA | NA | NA |
| | 47387914 | | | | |
| CALM3 | NA | NA | NA | NA | NA |
| CAMK1 | chr3: 9799031- | NA | NA | NA | NA |
| | 9799200 | | | | |
| CAMK2A | chr5: 149617852- | 899.232 | −2.988636 | uc010jhe.2 | chr5: 149617852- |
| | 149618050 | | | | 149618050 |
| CAMK2D | chr4: 114372188- | 321.046 | −9.882362 | uc003ibi.3 | chr4: 114372188- |
| | 114375667 | | | | 114375671 |
| CAMK4 | NA | NA | NA | NA | NA |
| CAMKK2 | chr12: 121675495- | NA | NA | NA | NA |
| | 121678501 | | | | |
| CAMLG | NA | 463.124 | −4.343469 | uc003kzt.3 | chr5: 134086641- |
| | | | | | 134087850 |
| CAMTA1 | chr1: 6931826- | NA | NA | NA | NA |
| | 6932107 | | | | |
| CANT1 | NA | 901.027 | −9.066449 | uc002jwj.3 | chr17: 76987798- |
| | | | | | 76989631 |
| CAP1 | chr1: 40537206- | NA | NA | NA | NA |
| | 40538321 | | | | |
| CAPN7 | NA | NA | NA | NA | NA |
| CAPN9 | NA | 251.966 | −6.492262 | uc001htz.1 | chr1: 230937343- |
| | | | | | 230937749 |
| CAPNS1 | NA | NA | NA | NA | NA |
| CARD18 | chr11: 105008448- | NA | NA | NA | NA |
| | 105008825 | | | | |
| CASC5 | NA | 412.791 | −5.397901 | uc010bbs.1 | chr15: 40954387- |
| | | | | | 40954881 |
| CASP1 | NA | NA | NA | NA | NA |
| CASP3 | chr4: 185548850- | NA | NA | NA | NA |
| | 185550425 | | | | |
| CASP4 | chr11: 104813594- | NA | NA | NA | NA |
| | 104813700 | | | | |
| CASP8 | chr2: 202137383- | 1037.46 | −1.530748 | uc002uxp.1 | chr2: 202151318- |
| | 202137499 | | | | 202152434 |
| CASP9 | NA | 526.363 | −4.168148 | uc001awm.2 | chr1: 15817324- |
| | | | | | 15820278 |
| CASS4 | NA | NA | NA | NA | NA |
| CAST | NA | NA | NA | NA | NA |
| CATSPER2 | NA | NA | NA | NA | NA |
| CATSPERB | chr14: 92047118- | NA | NA | NA | NA |
| | 92047232 | | | | |
| CATSPERG | chr19: 38861433- | NA | NA | NA | NA |
| | 38861589 | | | | |
| CAV1 | NA | 633.928 | −6.945885 | uc010lkd.2 | chr7: 116199342- |
| | | | | | 116201239 |
| CAV2 | chr7: 116146176- | NA | NA | NA | NA |
| | 116148595 | | | | |
| GBLB | chr3: 105377109- | 787.309 | −9.141429 | uc003dwa.3 | chr3: 105377109- |
| | 105377813 | | | | 105377813 |
| CBLL1 | chr7: 107399624- | NA | NA | NA | NA |
| | 107402083 | | | | |
| CBLN3 | chr14: 24895740- | NA | NA | NA | NA |
| | 24896994 | | | | |
| CBR1 | NA | NA | NA | NA | NA |
| CBR4 | NA | NA | NA | NA | NA |
| CBWD1 | NA | NA | NA | NA | NA |
| CBWD2 | chr2: 114201445- | NA | NA | NA | NA |
| | 114201594 | | | | |
| CBX3 | chr7: 26251829- | 1769.39 | −3.716168 | uc003sxt.3 | chr7: 26251829- |
| | 26253227 | | | | 26253227 |
| CBX5 | NA | 261.382 | −5.735666 | uc001sfh.4 | chr12: 54624731- |
| | | | | | 54635538 |
| CBX7 | chr22: 39526779- | NA | NA | NA | NA |
| | 39529895 | | | | |
| CC2D2A | NA | 275.777 | −5.958384 | uc003gnq.4 | chr4: 1548291O- |
| | | | | | 15483887 |
| CCBL2 | NA | NA | NA | NA | NA |
| CCDC108 | NA | 756.554 | −7.49656 | uc002vjm.3 | chr2: 219882978- |
| | | | | | 219883717 |
| CCDC109B | NA | NA | NA | NA | NA |
| CCDC110 | chr4: 186378923- | NA | NA | NA | NA |
| | 186379256 | | | | |
| CCDC126 | NA | 274.292 | −5.603029 | uc003swl.3 | chr7: 23682735- |
| | | | | | 23684327 |
| CCDC134 | NA | NA | NA | NA | NA |
| CCDC144A | chr17: 16676844- | NA | NA | NA | NA |
| | 16678311 | | | | |
| CCDC144NL | NA | NA | NA | NA | NA |
| CCDC148 | NA | NA | NA | NA | NA |
| CCDC150 | NA | 218.738 | −4.855898 | uc002uto.1 | chr2: 197531656- |
| | | | | | 197532658 |
| CCDC152 | NA | 1009.55 | −10.198354 | uc003jmx.3 | chr5: 42799884- |
| | | | | | 42802539 |
| CCDC158 | chr4: 77234192- | NA | NA | NA | NA |
| | 77234322 | | | | |
| CCDC169 | chr13: 36801179- | NA | NA | NA | NA |
| | 36801337 | | | | |
| CCDC174 | NA | 505.172 | −7.032316 | uc003byw.3 | chr3: 14712702- |
| | | | | | 14714166 |
| CCDC175 | NA | 685.023 | −5.496666 | uc021rtw.1 | chr14: 59971785- |
| | | | | | 59971956 |
| CCDC176 | NA | 292.274 | −5.109962 | uc010tup.2 | chr14: 74531403- |
| | | | | | 74532796 |
| CCDC18 | NA | NA | NA | NA | NA |
| CCDC180 | NA | 456.933 | −3.535729 | uc004axf.3 | chr9: 100105850- |
| | | | | | 100105925 |
| CCDC184 | chr12: 48578491- | NA | NA | NA | NA |
| | 48579709 | | | | |
| CCDC25 | NA | 295.205 | −5.693168 | uc003xgc.3 | chr8: 27590833- |
| | | | | | 27593732 |
| CCDC3 | NA | 279.513 | −6.786744 | uc001ilq.1 | chr10: 12938625- |
| | | | | | 12940415 |
| CCDC34 | NA | NA | NA | NA | NA |
| CCDC36 | chr3: 49282296- | NA | NA | NA | NA |
| | 49283171 | | | | |
| CCDC58 | chr3: 122078436- | 1201.47 | −10.054687 | uc003eey.3 | chr3: 122078436- |
| | 122078715 | | | | 122078715 |
| CCDC6 | NA | 314.035 | −8.067419 | uc001jks.4 | chr10: 61548506- |
| | | | | | 61552674 |
| CCDC60 | NA | NA | NA | NA | NA |
| CCDC66 | chr3: 56603391- | NA | NA | NA | NA |
| | 56604774 | | | | |
| CCDC7 | NA | 626.628 | −6.763677 | uc001iwl.1 | chr10: 33165322- |
| | | | | | 33165508 |
| CCDC70 | NA | NA | NA | NA | NA |
| CCDC73 | NA | 532.296 | −5.044439 | uc009yjt.3 | chr11: 32701408- |
| | | | | | 32705033 |
| CCDC80 | NA | NA | NA | NA | NA |
| CCDC81 | chr11: 86133758- | 961.096 | −10.50038 | uc001pbw.2 | chr11: 86133758- |
| | 86134151 | | | | 86134151 |
| CCDC83 | chr11: 85630554- | NA | NA | NA | NA |
| | 85631051 | | | | |
| CCDC88A | NA | 429.525 | −7.791389 | uc002rys.3 | chr2: 55515313- |
| | | | | | 55515491 |
| CCDC88B | chr11: 64117198- | NA | NA | NA | NA |
| | 64118167 | | | | |
| CCDC88C | chr14: 91737667- | 252.176 | −8.644124 | uc010twk.1 | chr14: 91805454- |
| | 91738968 | | | | 91805617 |
| CCDC89 | NA | NA | NA | NA | NA |
| CCDC9 | chr19: 47774936- | NA | NA | NA | NA |
| | 47775210 | | | | |
| CCDC90B | chr11: 82972502- | 839.29 | −7.592701 | uc001pac.3 | chr11: 82972502- |
| | 82972952 | | | | 82972952 |
| CCDC91 | NA | NA | NA | NA | NA |
| CCDC96 | chr4: 7042576- | NA | NA | NA | NA |
| | 7042997 | | | | |
| CCK | NA | 1627.99 | −11.696693 | uc021wwk.1 | chr3: 42299318- |
| | | | | | 42299589 |
| CCL1 | chr17: 32687399- | NA | NA | NA | NA |
| | 32687577 | | | | |
| CCL2 | NA | NA | NA | NA | NA |
| CCL20 | NA | NA | NA | NA | NA |
| CCL23 | NA | NA | NA | NA | NA |
| CCL25 | chr19: 8127110- | NA | NA | NA | NA |
| | 8127547 | | | | |
| CCL5 | NA | 1139.2 | −8.620127 | uc002hkf.3 | chr17: 34198496- |
| | | | | | 34199380 |
| CCNB1 | NA | NA | NA | NA | NA |
| CCNB2 | chr15: 59417077- | 1273.56 | −15.112176 | uc002afz.3 | chr15: 59417077- |
| | 59417244 | | | | 59417244 |
| CCNB3 | NA | 1507.84 | −17.585865 | uc004dox.4 | chrX: 50094703- |
| | | | | | 50094911 |
| CCND2 | chr12: 4409176- | 836.409 | −7.285364 | uc001qmo.3 | chr12: 4409176- |
| | 4414522 | | | | 4414522 |
| CCNDBP1 | chr15: 43487040- | NA | NA | NA | NA |
| | 43489375 | | | | |
| CCNE1 | NA | NA | NA | NA | NA |
| CCNF | chr16: 2507022- | NA | NA | NA | NA |
| | 2508859 | | | | |
| CCNG2 | chr4: 78087078- | NA | NA | NA | NA |
| | 78091213 | | | | |
| CCNJ | NA | 442.899 | −5.757108 | uc001klm.3 | chr10: 97817999- |
| | | | | | 97820625 |
| CCNJL | NA | 424.337 | −5.603029 | uc003lyb.1 | chr5: 159678671- |
| | | | | | 159680384 |
| CCNK | chr14: 99977120- | NA | NA | NA | NA |
| | 99977852 | | | | |
| CCNL1 | NA | 408.234 | −5.706638 | uc003fbe.3 | chr3: 156865586- |
| | | | | | 156866029 |
| CCNT1 | chr12: 49082241- | 741.912 | −9.240699 | uc001rsd.4 | chr12: 49082241- |
| | 49086815 | | | | 49086815 |
| CCNT2 | NA | 618.928 | −8.150906 | uc002tub.2 | chr2: 135712119- |
| | | | | | 135716915 |
| CCNY | chr10: 35858099- | NA | NA | NA | NA |
| | 35860847 | | | | |
| CCR2 | chr3: 46400102- | NA | NA | NA | NA |
| | 46400868 | | | | |
| CCRN4L | NA | 385.327 | −8.607589 | uc003ihk.1 | chr4: 139964542- |
| | | | | | 139965034 |
| CCSER2 | NA | NA | NA | NA | NA |
| CCT2 | chr12: 69995106- | NA | NA | NA | NA |
| | 69995357 | | | | |
| CCT5 | chr5: 10261984- | NA | NA | NA | NA |
| | 10262313 | | | | |
| CCT7 | chr2: 73479990- | 845.417 | −5.525511 | uc002siz.3 | chr2: 73479990- |
| | 73480150 | | | | 73480150 |
| CCT8 | chr21: 30428648- | NA | NA | NA | NA |
| | 30428795 | | | | |
| CCT8L2 | chr22: 17071648- | NA | NA | NA | NA |
| | 17071766 | | | | |
| CCZ1B | chr7: 6862353- | 576.764 | −8.608823 | uc003sqy.2 | chr7: 6862353- |
| | 6864043 | | | | 6864043 |
| CD164 | NA | NA | NA | NA | NA |
| CD1A | NA | 968.03 | −6.625238 | uc001frt.3 | chr1: 158227481- |
| | | | | | 158228058 |
| CD1C | chr1: 158263267- | NA | NA | NA | NA |
| | 158264564 | | | | |
| CD1E | NA | NA | NA | NA | NA |
| CD200R1 | chr3: 112641532- | NA | NA | NA | NA |
| | 112642523 | | | | |
| CD200R1L | chr3: 112534556- | 652.152 | −4.150229 | uc003dzi.1 | chr3: 112534556- |
| | 112534809 | | | | 112534809 |
| CD226 | chr18: 67530193- | NA | NA | NA | NA |
| | 67531549 | | | | |
| CD244 | chr1: 160799950- | 546.605 | −4.770657 | uc001fxa.3 | chr1: 160799950- |
| | 160801136 | | | | 160801136 |
| CD2AP | chr6: 47591964- | 665.828 | −7.207507 | uc003oyw.3 | chr6: 47591964- |
| | 47594996 | | | | 47594996 |
| CD2BP2 | chr16: 30362087- | NA | NA | NA | NA |
| | 30364305 | | | | |
| CD300LG | chr17: 41935197- | NA | NA | NA | NA |
| | 41935602 | | | | |
| CD33 | NA | 540.17 | −6.616247 | uc010eos.1 | chr19: 51739087- |
| | | | | | 51742993 |
| CD36 | NA | 1213.82 | −13.779237 | uc003uhc.3 | chr7: 80303464- |
| | | | | | 80303734 |
| CD40LG | chrX: 135741575- | 693.856 | −9.392037 | uc004faa.3 | chrX: 135741575- |
| | 135742549 | | | | 135742549 |
| CD55 | chr1: 207532956- | 1172.59 | −4.162373 | uc001hfq.4 | chr1: 207532956- |
| | 207534311 | | | | 207534311 |
| CD58 | NA | 488.634 | −5.017189 | uc010owy.2 | chr1: 117061322- |
| | | | | | 117061848 |
| CD69 | chr12: 9905082- | NA | NA | NA | NA |
| | 9906076 | | | | |
| CD72 | chr9: 35609976- | NA | NA | NA | NA |
| | 35610297 | | | | |
| CD79A | NA | 911.382 | −6.954196 | uc002oru.3 | chr19: 42385048- |
| | | | | | 42385439 |
| CD80 | chr3: 119243140- | NA | NA | NA | NA |
| | 119244596 | | | | |
| CD9 | NA | 219.973 | −18.577082 | uc001qnq.2 | chr12: 6346995- |
| | | | | | 6347437 |
| CD99L2 | NA | NA | NA | NA | NA |
| CDADC1 | NA | 637.04 | −6.276476 | uc001vcu.3 | chr13: 49865894- |
| | | | | | 49867622 |
| CDC123 | NA | 1351.49 | −6.676111 | uc009xja.2 | chr10: 12257853- |
| | | | | | 12258491 |
| CDC14A | chr1: 100950023- | 1626.37 | −4.732965 | uc009wec.1 | chr1: 100855495- |
| | 100950926 | | | | 100855515 |
| CDC16 | NA | 786.91 | −1.065824 | uc010tkm.1 | chr13: 115007791- |
| | | | | | 115008823 |
| CDC20 | chr1: 43828801- | 672.955 | −8.126236 | NA | NA |
| | 43828873 | | | | |
| CDC23 | NA | NA | NA | NA | NA |
| CDC37L1 | chr9: 4706113- | 1497.94 | −15.378742 | uc003zio.3 | chr9: 4706113- |
| | 4706594 | | | | 4706594 |
| CDC40 | NA | 1024.92 | −7.272968 | uc003pua.3 | chr6: 110551335- |
| | | | | | 110553422 |
| CDC42SE2 | NA | 672.539 | −8.287405 | uc003kvh.3 | chr5: 130726785- |
| | | | | | 130730360 |
| CDC5L | NA | 1213.04 | −9.123901 | uc003oxl.3 | chr6: 44414449- |
| | | | | | 44418161 |
| CDC7 | NA | 479.001 | −4.762079 | uc001doe.3 | chr1: 91989993- |
| | | | | | 91991321 |
| CDCA4 | chr14: 105475910- | NA | NA | NA | NA |
| | 105476970 | | | | |
| CDCA5 | NA | NA | NA | NA | NA |
| CDCA7L | chr7: 21940517- | NA | NA | NA | NA |
| | 21941939 | | | | |
| CDCP1 | chr3: 45123769- | NA | NA | NA | NA |
| | 45127129 | | | | |
| CDH1 | chr16: 68867403- | 433.341 | −4.362234 | uc002ewg.1 | chr16: 68867403- |
| | 68869444 | | | | 68869444 |
| CDH13 | chr16: 83214475- | 259.58 | −4.997563 | uc010chh.3 | chr16: 83214565- |
| | 83214630 | | | | 83214630 |
| CDH17 | chr8: 95139394- | NA | NA | NA | NA |
| | 95140467 | | | | |
| CDH18 | chr5: 19473155- | 1195.72 | −12.032067 | NA | NA |
| | 19473825 | | | | |
| CDH23 | chr10: 73377238- | NA | NA | NA | NA |
| | 73377362 | | | | |
| CDH5 | chr16: 66426291- | NA | NA | NA | NA |
| | 66426888 | | | | |
| CDH6 | NA | 269.379 | −7.120567 | uc003jhd.2 | chr5: 31318142- |
| | | | | | 31318435 |
| CDH7 | chr18: 63548131- | NA | NA | NA | NA |
| | 63548175 | | | | |
| CDH8 | NA | NA | NA | NA | NA |
| CDK1 | chr10: 62553734- | NA | NA | NA | NA |
| | 62554610 | | | | |
| CDK13 | chr7: 40134580- | NA | NA | NA | NA |
| | 40136733 | | | | |
| CDK17 | chr12: 96672039- | NA | NA | NA | NA |
| | 96674019 | | | | |
| CDK5RAP1 | NA | NA | NA | NA | NA |
| CDKL3 | chr5: 133541305- | 979.568 | −10.284501 | uc011cxq.2 | chr5: 133634115- |
| | 133541558 | | | | 133634401 |
| CDKL4 | NA | 580.947 | −7.50327 | uc002rrm.3 | chr2: 39405688- |
| | | | | | 39406306 |
| CDKN1A | chr6: 36653578- | 216.346 | −6.173846 | uc021yzb.1 | chr6: 36653578- |
| | 36655116 | | | | 36655116 |
| CDKN1B | NA | 469.435 | −7.506124 | uc001rat.2 | chr12: 12873970- |
| | | | | | 12875305 |
| CDKN1C | chr11: 2904448- | 458.424 | −4.006339 | uc001lwr.4 | chr11: 2904448- |
| | 2905140 | | | | 2905140 |
| CDKN2AIP | NA | NA | NA | NA | NA |
| CDKN2AIPNL | NA | 442.417 | −9.622014 | uc011cxs.2 | chr5: 133737756- |
| | | | | | 133738605 |
| CDKN2C | NA | 410.212 | −4.122947 | uc001csf.3 | chr1: 51439943- |
| | | | | | 51440309 |
| CDON | chr11: 125826713- | NA | NA | NA | NA |
| | 125830836 | | | | |
| CDR2 | NA | 706.237 | −6.08278 | uc002dkn.3 | chr16: 22357257- |
| | | | | | 22358285 |
| CDSN | chr6_dbb_hap3: | NA | NA | NA | NA |
| | 2380037-2380970 | | | | |
| CDV3 | chr3: 133306019- | NA | NA | NA | NA |
| | 133309118 | | | | |
| CDX1 | NA | 848.736 | −6.745981 | uc003lrq.3 | chr5: 149563244- |
| | | | | | 149564121 |
| CEACAM1 | chr19: 43011458- | 901.716 | −1.504843 | uc002otv.3 | chr19: 43011458- |
| | 43013260 | | | | 43013260 |
| CEACAM21 | NA | NA | NA | NA | NA |
| CEACAM4 | chr19: 42125344- | NA | NA | NA | NA |
| | 42125702 | | | | |
| CEACAM5 | chr19: 42219414- | NA | NA | NA | NA |
| | 42219789 | | | | |
| CEACAM8 | chr19: 43084395- | NA | NA | NA | NA |
| | 43085505 | | | | |
| CEBPZ | NA | NA | NA | NA | NA |
| CELA3B | chr1: 22315773- | NA | NA | NA | NA |
| | 22315847 | | | | |
| CELF5 | NA | 648.143 | −5.757253 | uc002lxl.2 | chr19: 3293333- |
| | | | | | 3297073 |
| CELSR2 | chr1: 109816672- | NA | NA | NA | NA |
| | 109818378 | | | | |
| CENPC | chr4: 68337989- | 413.617 | −8.771709 | uc003hdd.1 | chr4: 68337989- |
| | 68338322 | | | | 68338322 |
| CENPE | NA | 731.896 | −6.120114 | uc003hxb.1 | chr4: 104026963- |
| | | | | | 104027378 |
| CENPH | chr5: 68505627- | NA | NA | NA | NA |
| | 68506184 | | | | |
| CENPQ | chr6: 49459989- | NA | NA | NA | NA |
| | 49460820 | | | | |
| CENPV | NA | NA | NA | NA | NA |
| CENPW | NA | 1070.06 | −9.372888 | uc003qao.3 | chr6: 126669639- |
| | | | | | 126669754 |
| CEP120 | chr5: 122680579- | NA | NA | NA | NA |
| | 122682212 | | | | |
| CEP135 | NA | NA | NA | NA | NA |
| CEP152 | NA | NA | NA | NA | NA |
| CEP162 | chr6: 84833960- | NA | NA | NA | NA |
| | 84834788 | | | | |
| CEP164 | NA | NA | NA | NA | NA |
| CEP170 | NA | NA | NA | NA | NA |
| CEP290 | NA | NA | NA | NA | NA |
| CEP44 | chr4: 175238535- | 250.357 | −5.795954 | uc010iro.2 | chr4: 175252747- |
| | 175241482 | | | | 175254531 |
| CEP55 | chr10: 95287911- | NA | NA | NA | NA |
| | 95288849 | | | | |
| CEP57 | chr11: 95555149- | NA | NA | NA | NA |
| | 95556379 | | | | |
| CEP57L1 | NA | NA | NA | NA | NA |
| CEP63 | chr3: 134293734- | NA | NA | NA | NA |
| | 134293855 | | | | |
| CEP70 | chr3: 138213187- | NA | NA | NA | NA |
| | 138213856 | | | | |
| CEP72 | chr5: 653269- | NA | NA | NA | NA |
| | 653666 | | | | |
| CEP78 | chr9: 80881679- | NA | NA | NA | NA |
| | 80881983 | | | | |
| CEP83 | NA | 203.578 | −1.861905 | uc001tdd.3 | chr12: 94702056- |
| | | | | | 94702588 |
| CEP89 | NA | NA | NA | NA | NA |
| CEP97 | NA | NA | NA | NA | NA |
| CERS3 | chr15: 100940600- | NA | NA | NA | NA |
| | 100942917 | | | | |
| CES1P1 | chr16: 55807487- | 906.605 | −8.752191 | uc002eik.3 | chr16: 55807487- |
| | 55808826 | | | | 55808826 |
| CES5A | chr16: 55880066- | 953.872 | −8.866658 | uc002eio.2 | chr16: 55880066- |
| | 55880362 | | | | 55880362 |
| CETN1 | chr18: 580928- | NA | NA | NA | NA |
| | 581524 | | | | |
| CETN2 | chrX: 151995871- | NA | NA | NA | NA |
| | 151996384 | | | | |
| CETN3 | NA | NA | NA | NA | NA |
| CFAP20 | chr16: 58147497- | NA | NA | NA | NA |
| | 58147928 | | | | |
| CFAP43 | chr10: 105889646- | 528.11 | −5.490174 | uc009xxq.3 | chr10: 105889646- |
| | 105889896 | | | | 105889896 |
| CFAP44 | chr3: 113005777- | 813.92 | −8.334461 | uc010hqj.2 | chr3: 113005777- |
| | 113010403 | | | | 113010403 |
| CFAP45 | chr1: 159842154- | NA | NA | NA | NA |
| | 159842274 | | | | |
| CFAP47 | chrX: 36162927- | NA | NA | NA | NA |
| | 36163187 | | | | |
| CFAP53 | chr18: 47753563- | NA | NA | NA | NA |
| | 47753750 | | | | |
| CFAP61 | chr20: 20341055- | NA | NA | NA | NA |
| | 20341346 | | | | |
| CFAP70 | NA | 458.263 | −5.253225 | uc001jtv.4 | chr10: 75013516- |
| | | | | | 75013732 |
| CFAP97 | NA | 988.725 | −9.591056 | uc003ixf.4 | chr4: 186080819- |
| | | | | | 186083951 |
| CFH | chr1: 196716444- | 930.267 | −9.491429 | uc001gtj.4 | chr1: 196716444- |
| | 196716634 | | | | 196716634 |
| CFHR1 | chr1: 196801130- | 1419.92 | −15.580586 | uc001gtm.3 | chr1: 196801130- |
| | 196801319 | | | | 196801319 |
| CFHR2 | NA | 2031.6 | −20.389126 | uc001gtq.1 | chr1: 196928211- |
| | | | | | 196928356 |
| CFHR3 | NA | NA | NA | NA | NA |
| CFHR5 | chr1: 196977814- | NA | NA | NA | NA |
| | 196978803 | | | | |
| CFI | chr4: 110661848- | NA | NA | NA | NA |
| | 110662048 | | | | |
| CFP | NA | 201.186 | −6.706005 | uc004dig.4 | chrX: 47483612- |
| | | | | | 47483673 |
| CGA | NA | 982.272 | −5.610066 | uc003plj.2 | chr6: 87795216- |
| | | | | | 87795473 |
| CGGBP1 | NA | NA | NA | NA | NA |
| CHAC2 | chr2: 54001663- | NA | NA | NA | NA |
| | 54002287 | | | | |
| CHCHD4 | NA | 1029.72 | −13.224546 | uc003byi.4 | chr3: 14153577- |
| | | | | | 14154386 |
| CHD1 | NA | 887.816 | −6.179804 | uc010jbn.3 | chr5: 98190908- |
| | | | | | 98192083 |
| CHD2 | chr15: 93486366- | NA | NA | NA | NA |
| | 93488095 | | | | |
| CHD6 | chr20: 40064665- | NA | NA | NA | NA |
| | 40065865 | | | | |
| CHD8 | NA | 386.421 | −5.409259 | uc001war.2 | chr14: 21853353- |
| | | | | | 21853771 |
| CHEK2 | NA | NA | NA | NA | NA |
| CHGB | NA | 215.73 | −4.895349 | uc002wmg.3 | chr20: 5905696- |
| | | | | | 5906005 |
| CHIC2 | chr4: 54875958- | 741.821 | −7.162204 | uc003haj.2 | chr4: 54875958- |
| | 54876261 | | | | 54876261 |
| CHKA | NA | 454.913 | −5.439775 | uc001onj.3 | chr11: 67820326- |
| | | | | | 67821454 |
| CHMP2B | NA | 572.065 | −8.501789 | uc003dqp.4 | chr3: 87302973- |
| | | | | | 87304698 |
| CHMP4A | chr14: 24678787- | NA | NA | NA | NA |
| | 24679082 | | | | |
| CHMP6 | NA | NA | NA | NA | NA |
| CHMP7 | NA | NA | NA | NA | NA |
| CHODL | chr21: 19638356- | NA | NA | NA | NA |
| | 19639687 | | | | |
| CHORDC1 | NA | 231.139 | −11.746528 | uc001pdg.2 | chr11: 89933597- |
| | | | | | 89935572 |
| CHPT1 | chr12: 102122724- | 710.395 | −1.826518 | uc001tin.3 | chr12: 102122724- |
| | 102122846 | | | | 102122846 |
| CHRDL2 | chr11: 74407474- | 617.335 | −12.403018 | uc001ovh.3 | chr11: 74407474- |
| | 74407553 | | | | 74407542 |
| CHRM3 | NA | NA | NA | NA | NA |
| CHRM5 | NA | NA | NA | NA | NA |
| CHRNG | NA | NA | NA | NA | NA |
| CHST11 | chr12: 105151582- | NA | NA | NA | NA |
| | 105155792 | | | | |
| CHST12 | NA | 1264.8 | −5.855737 | uc003smc.3 | chr7: 2473520- |
| | | | | | 2474216 |
| CHST8 | NA | 461.506 | −5.143917 | uc002nus.4 | chr19: 34263969- |
| | | | | | 34264414 |
| CHSY1 | NA | 1552.64 | −10.659597 | uc010usd.2 | chr15: 101715928- |
| | | | | | 101717592 |
| CHSY3 | NA | NA | NA | NA | NA |
| CHTF8 | NA | 646.261 | −6.058024 | uc002ewo.2 | chr16: 69151912- |
| | | | | | 69152756 |
| CHTOP | chr1: 153614926- | NA | NA | NA | NA |
| | 153615872 | | | | |
| CHURC1 | NA | 1806.37 | −29.380444 | uc001xhw.2 | chr14: 65398949- |
| | | | | | 65402084 |
| CIAPIN1 | NA | NA | NA | NA | NA |
| CIDEA | chr18: 12277270- | NA | NA | NA | NA |
| | 12277594 | | | | |
| CILP | NA | NA | NA | NA | NA |
| CIPC | NA | 489.28 | −5.190314 | uc001xtc.1 | chr14: 77580662- |
| | | | | | 77583629 |
| CIR1 | NA | NA | NA | NA | NA |
| CIRH1A | NA | NA | NA | NA | NA |
| CISD2 | chr4: 103808588- | 731.843 | −14.229902 | uc003hwt.4 | chr4: 103808588- |
| | 103813963 | | | | 103813963 |
| CKAP2 | chr13: 53049277- | NA | NA | NA | NA |
| | 53050763 | | | | |
| CKLF | chr16: 66599915- | NA | NA | NA | NA |
| | 66600190 | | | | |
| CKMT1B | chr15: 43889595- | NA | NA | NA | NA |
| | 43890525 | | | | |
| CKS1B | chr1: 154951254- | 1182.07 | −11.469686 | uc001fgb.3 | chr1: 154951254- |
| | 154951725 | | | | 154951725 |
| CKS2 | NA | NA | NA | NA | NA |
| CLASP1 | chr2: 122095352- | 297.035 | −3.982861 | uc002tng.1 | chr2: 122159085- |
| | 122098436 | | | | 122159198 |
| CLASP2 | chr3: 33537739- | NA | NA | NA | NA |
| | 33540122 | | | | |
| CLCA1 | NA | 1451.35 | −11.833309 | uc001dlt.3 | chr1: 86965729- |
| | | | | | 86965974 |
| CLCN3 | chr4: 170641217- | NA | NA | NA | NA |
| | 170644338 | | | | |
| CLDN11 | NA | 322.047 | −4.711478 | uc011bpt.1 | chr3: 170577772- |
| | | | | | 170578169 |
| CLDN14 | chr21: 37832920- | NA | NA | NA | NA |
| | 37833273 | | | | |
| CLDN22 | NA | NA | NA | NA | NA |
| CLDN6 | chr16: 3064713- | NA | NA | NA | NA |
| | 3065359 | | | | |
| CLDN7 | chr17: 7163222- | NA | NA | NA | NA |
| | 7163692 | | | | |
| CLDN8 | chr21: 31586324- | NA | NA | NA | NA |
| | 31587565 | | | | |
| CLDND1 | chr3: 98234317- | NA | NA | NA | NA |
| | 98235502 | | | | |
| CLEC18B | NA | NA | NA | NA | NA |
| CLEC4A | chr12: 8290884- | NA | NA | NA | NA |
| | 8291203 | | | | |
| CLEC4D | NA | 454.349 | −6.947905 | uc001qun.3 | chr12: 8673868- |
| | | | | | 8674960 |
| CLEC6A | NA | 468.919 | −5.694385 | uc001qum.1 | chr12: 8630061- |
| | | | | | 8630926 |
| CLEC9A | NA | 796.138 | −12.246875 | uc001qxa.3 | chr12: 10218232- |
| | | | | | 10218565 |
| CLECL1 | chr12: 9875088- | NA | NA | NA | NA |
| | 9875221 | | | | |
| CLIC2 | chrX: 154505500- | NA | NA | NA | NA |
| | 154507191 | | | | |
| CLIC6 | NA | 275.869 | −3.711993 | uc002yuf.1 | chr21: 36088781- |
| | | | | | 36090519 |
| CLK1 | chr2: 201717732- | NA | NA | NA | NA |
| | 201718028 | | | | |
| CLK2 | chr1: 155232659- | NA | NA | NA | NA |
| | 155233008 | | | | |
| CLK2P1 | chr7: 23624335- | NA | NA | NA | NA |
| | 23624680 | | | | |
| CLK3 | NA | 210.291 | −13.783986 | uc002ayg.4 | chr15: 74922225- |
| | | | | | 74922542 |
| CLK4 | chr5: 178029665- | 522.819 | −9.184256 | uc003mjf.1 | chr5: 178029665- |
| | 178030758 | | | | 178030617 |
| CLMN | NA | NA | NA | NA | NA |
| CLMP | NA | 237.573 | −4.270358 | uc001pyt.3 | chr11: 122943033- |
| | | | | | 122944181 |
| CLN5 | NA | NA | NA | NA | NA |
| CLNK | chr4: 10491838- | 1596.82 | −9.065956 | uc003gmo.4 | chr4: 10491838- |
| | 10492090 | | | | 10492090 |
| CLPB | NA | 795.914 | −4.801302 | uc001osi.3 | chr11: 72003470- |
| | | | | | 72004410 |
| CLRN1 | chr3: 150643950- | NA | NA | NA | NA |
| | 150644638 | | | | |
| CLRN3 | NA | NA | NA | NA | NA |
| CLTA | NA | NA | NA | NA | NA |
| CMC2 | NA | NA | NA | NA | NA |
| CMPK | NA | 699.876 | −6.237268 | uc001cri.3 | chr11: 47842418- |
| | | | | | 47844511 |
| CMSS1 | NA | 292.506 | −10.150883 | uc003dtl.3 | chr3: 99897233- |
| | | | | | 99897476 |
| CMTM2 | NA | 1921.47 | −10.74859 | uc002ept.3 | chr16: 66622023- |
| | | | | | 66622177 |
| CMTR2 | NA | 536.164 | −8.640653 | uc010cga.3 | chr16: 71316203- |
| | | | | | 71317510 |
| CNGA1 | NA | 929.223 | −7.301824 | uc003gxu.3 | chr4: 47937994- |
| | | | | | 47938437 |
| CNIH1 | NA | 357.459 | −5.022506 | uc001xat.1 | chr14: 54893647- |
| | | | | | 54894531 |
| CNKSR3 | NA | NA | NA | NA | NA |
| CNN2 | chr19: 1037900- | NA | NA | NA | NA |
| | 1039064 | | | | |
| CNN3 | NA | NA | NA | NA | NA |
| CNOT4 | chr7: 135071822- | NA | NA | NA | NA |
| | 135073545 | | | | |
| CNOT6L | chr4: 78634541- | NA | NA | NA | NA |
| | 78641584 | | | | |
| CNOT7 | NA | NA | NA | NA | NA |
| CNP | NA | 1149.9 | −11.293273 | uc002hyl.1 | chr17: 40125943- |
| | | | | | 40129754 |
| CNPY1 | chr7: 155293953- | NA | NA | NA | NA |
| | 155295762 | | | | |
| CNPY2 | NA | 584.411 | −5.446144 | uc001sku.2 | chr12: 56704213- |
| | | | | | 56704335 |
| CNRIP1 | NA | 652.619 | −1.407842 | Uc002sej.4 | chr2: 68511303- |
| | | | | | 68511559 |
| CNTD2 | chr19: 40728115- | NA | NA | NA | NA |
| | 40728958 | | | | |
| CNTF | NA | NA | NA | NA | NA |
| CNTLN | NA | 232.65 | −4.658954 | uc003zmw.2 | chr9: 17166979- |
| | | | | | 17167372 |
| CNTN3 | chr3: 74311722- | 1875.87 | −9.812763 | uc003dpm.1 | chr3: 74311722- |
| | 74313551 | | | | 74313551 |
| CNTN4 | chr3: 3097905- | 572.286 | −4.554289 | uc003bpd.1 | chr3: 3079021- |
| | 3099645 | | | | 3079360 |
| CNTN5 | chr11: 100095571- | NA | NA | NA | NA |
| | 100096179 | | | | |
| CNTN6 | chr3: 1339680- | 872.121 | −6.819105 | uc010hbo.2 | chr3: 1339680- |
| | 1340053 | | | | 1340053 |
| CNTRL | NA | NA | NA | NA | NA |
| CNTROB | NA | 739.839 | −11.600749 | uc002gjp.3 | chr17: 7852829- |
| | | | | | 7853237 |
| COA5 | NA | 672.932 | −6.641836 | uc002syz.3 | chr2: 99215786- |
| | | | | | 99217214 |
| COBLL1 | NA | 613.584 | −7.645332 | uc002ucn.3 | chr2: 165541258- |
| | | | | | 165542455 |
| COG4 | chr16: 70514472- | NA | NA | NA | NA |
| | 70514912 | | | | |
| COL12A1 | NA | 252.315 | −5.513544 | uc021zbv.1 | chr6: 75794042- |
| | | | | | 75796262 |
| COL13A1 | NA | 1033.2 | −13.771831 | uc001jqk.2 | chr10: 71718458- |
| | | | | | 71718904 |
| COL19A1 | NA | NA | NA | NA | NA |
| COL21A1 | NA | 814.409 | −5.710016 | uc003pcr.3 | chr6: 55921388- |
| | | | | | 55922454 |
| COL23A1 | NA | 754.306 | −9.498056 | uc010jkt.2 | chr5: 177554517- |
| | | | | | 177555598 |
| COL3A1 | NA | NA | NA | NA | NA |
| COL4A1 | NA | 1207.74 | −8.722427 | uc001vqw.4 | chr13: 110801310- |
| | | | | | 110802709 |
| COL4A2 | NA | 1152.19 | −9.710903 | uc001vqx.3 | chr13: 111154539- |
| | | | | | 111155373 |
| COL4A3 | NA | 573.829 | −6.356593 | uc002vom.2 | chr2: 228176587- |
| | | | | | 228179508 |
| COL4A3BP | NA | 660.308 | −7.147229 | uc003kds.3 | chr5: 74666928- |
| | | | | | 74570015 |
| COL6A3 | NA | NA | NA | NA | NA |
| COLEC10 | chr8: 120118431- | 859.994 | −15.435508 | uc003yoo.3 | chr8: 120118431- |
| | 120119202 | | | | 120119202 |
| COLGALT2 | chr1: 183904966- | 520.677 | −6.2762 | uc010poj.1 | chr1: 183898796- |
| | 183907894 | | | | 183899362 |
| COMMD10 | NA | 627.314 | −7.430117 | uc003krt.1 | chr5: 115628187- |
| | | | | | 115628978 |
| COMMD6 | NA | NA | NA | NA | NA |
| COMMD8 | NA | 797.599 | −6.784494 | uc003gxi.3 | chr4: 47452811- |
| | | | | | 47453661 |
| COPB2 | NA | 541.645 | −6.795238 | uc011bmv.2 | chr3: 139076433- |
| | | | | | 139076704 |
| COPS4 | chr4: 83996584- | NA | NA | NA | NA |
| | 83996971 | | | | |
| COPS8 | NA | NA | NA | NA | NA |
| COQ10B | chr2: 198338649- | 1357.3 | −8.998711 | uc002uuh.1 | chr2: 198338649- |
| | 198339851 | | | | 198339851 |
| COQ2 | NA | 511.471 | −5.290866 | uc003hog.3 | chr4: 84184977- |
| | | | | | 84185351 |
| COQ3 | chr6: 99817348- | NA | NA | NA | NA |
| | 99817475 | | | | |
| COQ4 | chr9: 131095925- | NA | NA | NA | NA |
| | 131096351 | | | | |
| CORIN | chr4: 47663505- | 399.012 | −4.708516 | uc011bzf.2 | chr4: 47596018- |
| | 47663722 | | | | 47597737 |
| CORO2A | NA | 472.595 | −4.362155 | uc004ayk.3 | chr9: 100883257- |
| | | | | | 100887055 |
| COX10 | chr17: 14110531- | NA | NA | NA | NA |
| | 14111996 | | | | |
| COX16 | chr14: 70791798- | 980.197 | −12.301669 | uc001xmb.3 | chr14: 70791798- |
| | 70793049 | | | | 70793049 |
| COX7A2 | NA | 720.478 | −5.458301 | uc003phv.2 | chr6: 75947391- |
| | | | | | 75947645 |
| COX7A2L | chr2: 42577645- | NA | NA | NA | NA |
| | 42578358 | | | | |
| COX7B2 | NA | 738.051 | −6.363592 | uc003gxf.3 | chr4: 46736847- |
| | | | | | 46736963 |
| COX8C | NA | NA | NA | NA | NA |
| CP | NA | NA | NA | NA | NA |
| CPA2 | NA | 645.651 | −10.389385 | uc011kpc.1 | chr7: 129916651- |
| | | | | | 129916872 |
| CPA6 | chr8: 68395517- | NA | NA | NA | NA |
| | 68395892 | | | | |
| CPAMD8 | chr19: 17003762- | NA | NA | NA | NA |
| | 17003918 | | | | |
| CPB1 | NA | 773.145 | −5.710244 | uc003ewl.3 | chr3: 148577790- |
| | | | | | 148577972 |
| CPB2 | NA | 1102.1 | −8.555592 | uc001vaw.3 | chr13: 46627322- |
| | | | | | 46627748 |
| CPEB4 | NA | NA | NA | NA | NA |
| CPLX2 | chr5: 175307049- | NA | NA | NA | NA |
| | 175311023 | | | | |
| CPLX3 | NA | NA | NA | NA | NA |
| CPN1 | NA | 514.886 | −8.522531 | NA | NA |
| CPNE1 | chr20: 34213953- | NA | NA | NA | NA |
| | 34214162 | | | | |
| CPNE2 | NA | NA | NA | NA | NA |
| CPNE3 | NA | 495.839 | −5.717069 | uc003ydv.2 | chr8: 87570639- |
| | | | | | 87573726 |
| CPNE4 | NA | NA | NA | NA | NA |
| CPPED1 | chr16: 12753656- | 803.495 | −6.259272 | uc002dca.4 | chr16: 12753656- |
| | 12758742 | | | | 12758742 |
| CPSF2 | chr14: 92628089- | NA | NA | NA | NA |
| | 92630543 | | | | |
| CPSF7 | NA | 655.151 | −5.175258 | uc001nrp.3 | chr11: 61170120- |
| | | | | | 61172176 |
| CPT1B | NA | 539.136 | −5.397706 | uc003bmk.4 | chr22: 51007290- |
| | | | | | 51007510 |
| CPTP | NA | NA | NA | NA | NA |
| CPZ | chr4: 8621345- | NA | NA | NA | NA |
| | 8621488 | | | | |
| CRABP1 | NA | 574.611 | −7.26967 | uc002bdp.2 | chr15: 78640320- |
| | | | | | 78640572 |
| CRACR2A | NA | 559.554 | −7.427761 | uc001qmj.2 | chr12: 3757109- |
| | | | | | 3757637 |
| CRCP | chr7: 65617345- | NA | NA | NA | NA |
| | 65619553 | | | | |
| CREB3L2 | NA | 630.499 | −4.112428 | uc003vtv.3 | chr7: 137559725- |
| | | | | | 137565221 |
| CREB5 | chr7: 28858897- | NA | NA | NA | NA |
| | 28865511 | | | | |
| CREBRF | chr5: 172560749- | NA | NA | NA | NA |
| | 172566291 | | | | |
| CREM | NA | 569.903 | −3.870552 | uc001ixy.3 | chr10: 35500732- |
| | | | | | 35501886 |
| CRHBP | NA | NA | NA | NA | NA |
| CRIPAK | NA | 316.176 | −5.505275 | uc003gdf.2 | chr4: 1389641- |
| | | | | | 1389782 |
| CRISP1 | chr6: 49801979- | 639.41 | −5.673029 | uc003ozw.2 | chr6: 49801979- |
| | 49803028 | | | | 49803028 |
| CRISPLD2 | chr16: 84872582- | 939.721 | −8.472077 | uc002fil.2 | chr16: 84879515- |
| | 84873626 | | | | 84879756 |
| CRK | chr17: 1324647- | NA | NA | NA | NA |
| | 1326806 | | | | |
| CRKL | chr22: 21304134- | 308.904 | −3.22987 | uc002ztf.2 | chr22: 21304134- |
| | 21308037 | | | | 21308037 |
| CRLF3 | chr17: 29109702- | 241.072 | −5.666301 | uc002hfr.4 | chr17: 29109702- |
| | 29111204 | | | | 29111204 |
| CRLS1 | chr20: 6017805- | NA | NA | NA | NA |
| | 6020697 | | | | |
| CRNKL1 | chr20: 20015012- | NA | NA | NA | NA |
| | 20016838 | | | | |
| CRNN | NA | 509.282 | −6.42302 | uc001ezx.2 | chr1: 152381719- |
| | | | | | 152382069 |
| CRTAP | NA | 373.917 | −5.428603 | uc003cfl.4 | chr3: 33183941- |
| | | | | | 33189265 |
| CRYAA | NA | 546.94 | −5.948126 | uc002zdd.1 | chr21: 44592391- |
| | | | | | 44592913 |
| CRYBA4 | NA | NA | NA | NA | NA |
| CRYGS | chr3: 186256232- | NA | NA | NA | NA |
| | 186256484 | | | | |
| CSAD | NA | NA | NA | NA | NA |
| CSF1R | NA | 688.995 | −7.060863 | uc011dcf.2 | chr5: 149456467- |
| | | | | | 149456695 |
| CSF2RA | NA | 807.893 | −13.307158 | uc004cpn.2 | chrX: 1428373- |
| | | | | | 1428828 |
| CSF3 | NA | 744.975 | −8.771447 | uc002hto.3 | chr17: 38173213- |
| | | | | | 38174066 |
| CSH2 | chr17: 61949372- | NA | NA | NA | NA |
| | 61949888 | | | | |
| CSMD1 | NA | 382.762 | −4.946611 | uc003wqe.3 | chr8: 3008768- |
| | | | | | 3008910 |
| CSMD3 | chr8: 113235159- | 892.109 | −8.439789 | uc003yns.3 | chr8: 113235159- |
| | 113236999 | | | | 113236999 |
| CSN1S2BP | NA | 547.029 | −4.831591 | uc021xoy.1 | chr4: 71012337- |
| | | | | | 71012421 |
| CSNK1D | chr17: 80200537- | NA | NA | NA | NA |
| | 80202707 | | | | |
| CSNK2B | chr6: 31636474- | 380.841 | −6.555534 | uc010jsz.1 | chr6: 31634966- |
| | 31636877 | | | | 31635607 |
| CSRNP2 | NA | 765.319 | −6.815313 | uc021qxx.1 | chr12: 51454988- |
| | | | | | 51457528 |
| CST9L | NA | 608.745 | −5.553976 | uc002wtk.4 | chr20: 23545370- |
| | | | | | 23545584 |
| CSTF2 | chrX: 100095703- | NA | NA | NA | NA |
| | 100095923 | | | | |
| CSTF2T | NA | 716.676 | −7.629105 | uc001jjp.3 | chr10: 53455246- |
| | | | | | 53457458 |
| CSTF3 | NA | NA | NA | NA | NA |
| CT45A4 | NA | 376.405 | −6.257567 | uc004ezb.3 | chrX: 134891327- |
| | | | | | 134891519 |
| CT55 | chrX: 134290461- | 812.478 | −5.088956 | uc004eyl.1 | chrX: 134290461- |
| | 134291236 | | | | 134291236 |
| CT62 | chr15: 71402583- | 264.099 | −3.291903 | uc002ata.2 | chr15: 71402583- |
| | 71403489 | | | | 71403489 |
| CTAG2 | chrX: 153880246- | NA | NA | NA | NA |
| | 153880541 | | | | |
| CTAGE5 | chr14: 39819469- | 1268.83 | −9.384304 | uc001wuy.4 | chr14: 39819469- |
| | 39820397 | | | | 39820397 |
| CTBS | NA | NA | NA | NA | NA |
| CTCF | NA | 376.228 | −4.807503 | uc002etl.3 | chr16: 67671776- |
| | | | | | 67673088 |
| CTCFL | NA | NA | NA | NA | NA |
| CTDSPL2 | NA | 689.39 | −6.756712 | uc001ztr.3 | chr15: 44816373- |
| | | | | | 44819429 |
| CTF1 | NA | 489.494 | −7.710261 | uc002dzw.3 | chr16: 30913861- |
| | | | | | 30914881 |
| CTNNBL1 | chr20: 36500416- | NA | NA | NA | NA |
| | 36500520 | | | | |
| CTNND2 | chr5: 10971952- | 906.646 | −7.361391 | uc011cmx.1 | chr5: 10971952- |
| | 10973564 | | | | 10973564 |
| CTPS1 | chr1: 41477330- | 861.018 | −4.395015 | uc001cgk.4 | chr1: 41477330- |
| | 41478235 | | | | 41478235 |
| CTRL | chr16: 67963473- | NA | NA | NA | NA |
| | 67963836 | | | | |
| CTSK | chr1: 150768684- | NA | NA | NA | NA |
| | 150769274 | | | | |
| CTSO | NA | 503.328 | −10.23806 | uc003ipg.3 | chr4: 156845270- |
| | | | | | 156847187 |
| CTSS | chr1: 150702672- | NA | NA | NA | NA |
| | 150705521 | | | | |
| CTSV | chr9: 99791959- | 1327.41 | −7.824636 | uc010msj.2 | chr9: 99796636- |
| | 99795230 | | | | 99796988 |
| CTTN | chr11: 70281269- | 546.925 | −1.59443 | uc001opu.3 | chr11: 70282515- |
| | 70282690 | | | | 70282690 |
| CTTNBP2 | chr7: 117350706- | NA | NA | NA | NA |
| | 117351590 | | | | |
| CTXN2 | chr15: 48493744- | 683.181 | −7.27264 | uc001zwm.1 | chr15: 48493744- |
| | 48495951 | | | | 48495951 |
| CUL2 | chr10: 35297479- | NA | NA | NA | NA |
| | 35299238 | | | | |
| CUL4B | NA | NA | NA | NA | NA |
| CUL5 | chr11: 107975112- | NA | NA | NA | NA |
| | 107978488 | | | | |
| CWC15 | chr11: 94704128- | NA | NA | NA | NA |
| | 94704491 | | | | |
| CWC22 | NA | 414.562 | −6.621505 | uc002uno.2 | chr2: 180809604- |
| | | | | | 180809855 |
| CX3CR1 | NA | NA | NA | NA | NA |
| CXADR | chr21: 18938011- | 950.181 | −3.520643 | uc002ykh.2 | chr21: 18937934- |
| | 18942429 | | | | 18942429 |
| CXCL10 | chr4: 76942269- | NA | NA | NA | NA |
| | 76943082 | | | | |
| CXCL13 | chr4: 78532194- | NA | NA | NA | NA |
| | 78532988 | | | | |
| CXCL16 | chr17: 4636828- | NA | NA | NA | NA |
| | 4637774 | | | | |
| CXCL2 | NA | 566.791 | −4.639055 | uc003hhm.4 | chr4: 74962754- |
| | | | | | 74963472 |
| CXCL8 | NA | 374.28 | −4.326565 | uc003hhe.3 | chr4: 74608182- |
| | | | | | 74609433 |
| CXCL9 | NA | 276.473 | −7.750099 | uc003hjh.1 | chr4: 76922623- |
| | | | | | 76924750 |
| CXCR5 | chr11: 118765373- | NA | NA | NA | NA |
| | 118766980 | | | | |
| CXCR6 | NA | 1139.81 | −7.122918 | uc003cpc.1 | chr3: 45989003- |
| | | | | | 45989845 |
| CXorf38 | chrX: 40486173- | NA | NA | NA | NA |
| | 40489414 | | | | |
| CXorf40B | NA | NA | NA | NA | NA |
| CYB561D1 | NA | NA | NA | NA | NA |
| CYB5B | NA | NA | NA | NA | NA |
| CYB5D1 | chr17: 7762931- | NA | NA | NA | NA |
| | 7765600 | | | | |
| CYB5R2 | chr11: 7686326- | 431.422 | −8.598565 | uc001mfm.3 | chr11: 7686326- |
| | 7686604 | | | | 7686604 |
| CYB5R4 | chr6: 84669598- | NA | NA | NA | NA |
| | 84670146 | | | | |
| CYFIP1 | NA | 641.022 | −8.560023 | uc001yus.3 | chr15: 23003041- |
| | | | | | 23003603 |
| CYLC2 | chr9: 105780607- | NA | NA | NA | NA |
| | 105780770 | | | | |
| CYLD | NA | 802.094 | −8.611595 | uc002ego.3 | chr16: 50818367- |
| | | | | | 50820674 |
| CYP19A1 | chr15: 51500254- | 303.837 | −3.677465 | uc001zyz.4 | chr15: 51500254- |
| | 51503004 | | | | 51503004 |
| CYP1A1 | NA | 1322.6 | −11.844586 | uc010bjz.1 | chr15: 75013061- |
| | | | | | 75013303 |
| CYP1A2 | chr15: 75047430- | NA | NA | NA | NA |
| | 75048941 | | | | |
| CYP1B1 | NA | 630.418 | −5.163575 | uc002rqo.2 | chr2: 38294746- |
| | | | | | 38297864 |
| CYP20A1 | NA | 943.659 | −9.285444 | uc002uzv.4 | chr2: 204161632- |
| | | | | | 204170563 |
| CYP21A2 | chr6: 32008912- | NA | NA | NA | NA |
| | 32009447 | | | | |
| CYP24A1 | chr20: 52769988- | NA | NA | NA | NA |
| | 52771300 | | | | |
| CYP27B1 | NA | 942.975 | −8.666374 | uc001sqb.1 | chr12: 58158161- |
| | | | | | 58158301 |
| CYP2C9 | chr10: 96748786- | 258.71 | −4.834918 | uc001kjz.3 | chr10: 96702107- |
| | 96749148 | | | | 96703022 |
| CYP2E1 | chr10: 135352469- | NA | NA | NA | NA |
| | 135352620 | | | | |
| CYP3A5 | chr7: 99268845- | 286.356 | −4.133205 | uc003urq.3 | chr7: 99245813- |
| | 99269396 | | | | 99245927 |
| CYP46A1 | chr14: 100174043- | NA | NA | NA | NA |
| | 100179774 | | | | |
| CYP4F12 | chr19: 15791495- | NA | NA | NA | NA |
| | 15791754 | | | | |
| CYP4F3 | NA | 343.624 | −6.258253 | uc002nbj.3 | chr19: 15770196- |
| | | | | | 15771570 |
| CYP4F8 | chr19: 15729198- | NA | NA | NA | NA |
| | 15730572 | | | | |
| CYP4V2 | chr4: 187131796- | NA | NA | NA | NA |
| | 187134617 | | | | |
| CYP4X1 | chr1: 47515847- | NA | NA | NA | NA |
| | 47516423 | | | | |
| CYP4Z1 | chr1: 47583607- | NA | NA | NA | NA |
| | 47583992 | | | | |
| CYP51A1 | chr7: 91741463- | NA | NA | NA | NA |
| | 91742978 | | | | |
| CYSLTR2 | chr13: 49281995- | NA | NA | NA | NA |
| | 49283498 | | | | |
| CYTH1 | NA | NA | NA | NA | NA |
| CYTH2 | NA | NA | NA | NA | NA |
| CYTIP | NA | NA | NA | NA | NA |
| CYYR1 | NA | 541.551 | −4.28772 | uc002ymd.3 | chr21: 27838528- |
| | | | | | 27840819 |
| DACT1 | NA | 244.938 | −5.936957 | uc010trv.2 | chr14: 59113853- |
| | | | | | 59115038 |
| DACT2 | NA | 225.831 | −8.16927 | uc003qwq.3 | chr6: 168707584- |
| | | | | | 168708111 |
| DAND5 | chr19: 13084203- | NA | NA | NA | NA |
| | 13085567 | | | | |
| DAPK1 | NA | NA | NA | NA | NA |
| DAPP1 | chr4: 100789343- | 568.437 | −5.988957 | uc011cek.2 | chr4: 100784927- |
| | 100791346 | | | | 100785442 |
| DARS | NA | NA | NA | NA | NA |
| DAZL | NA | 654.627 | −5.189693 | uc003cba.3 | chr3: 16628301- |
| | | | | | 16630166 |
| DBF4 | NA | NA | NA | NA | NA |
| DBI | NA | 1064.77 | −10.898804 | uc002tlv.3 | chr2: 120129895- |
| | | | | | 120130122 |
| DBR1 | chr3: 137879830- | NA | NA | NA | NA |
| | 137880730 | | | | |
| DCAF12 | NA | 1216.33 | −8.258806 | uc003ztt.2 | chr9: 34086381- |
| | | | | | 34088347 |
| DCAF12L2 | chrX: 125297482- | NA | NA | NA | NA |
| | 125298515 | | | | |
| DCAF13 | NA | NA | NA | NA | NA |
| DCAF16 | chr4: 17802278- | NA | NA | NA | NA |
| | 17805113 | | | | |
| DCAF5 | NA | 1379.79 | −9.270454 | uc001xkp.3 | chr14: 69517637- |
| | | | | | 69520573 |
| DCAF6 | NA | 632.703 | −8.611271 | uc001gev.3 | chr1: 168044674- |
| | | | | | 168045083 |
| DCAKD | NA | NA | NA | NA | NA |
| DCBLD1 | chr6: 117869070- | NA | NA | NA | NA |
| | 117870960 | | | | |
| DCBLD2 | NA | 624.529 | −7.237003 | uc003dtd.3 | chr3: 98514814- |
| | | | | | 98518215 |
| DCK | chr4: 71895096- | 480.265 | −5.773908 | uc003hfx.3 | chr4: 71895096- |
| | 71896629 | | | | 71896629 |
| DCLK1 | chr13: 36342789- | 526.546 | −7.81021 | uc001uve.4 | chr13: 36342789- |
| | 36348778 | | | | 36348778 |
| DCN | NA | 850.482 | −11.832845 | uc001tbo.3 | chr12: 91539035- |
| | | | | | 91539834 |
| DCP1B | NA | 869.9 | 0.089781 | uc010sdy.1 | chr12: 2050757- |
| | | | | | 2050957 |
| DCT | chr13: 95091841- | NA | NA | NA | NA |
| | 95092151 | | | | |
| DCTD | NA | 442.989 | −8.067711 | uc003ivf.3 | chr4: 183811244- |
| | | | | | 183812551 |
| DCTPP1 | NA | 550.497 | −5.531388 | uc002dyf.3 | chr16: 30435019- |
| | | | | | 30435553 |
| DCUN1D1 | NA | 376.573 | −8.960392 | uc003fld.1 | chr3: 182660559- |
| | | | | | 182662881 |
| DCX | chrX: 110537007- | NA | NA | NA | NA |
| | 110544914 | | | | |
| DDAH1 | NA | NA | NA | NA | NA |
| DDHD1 | NA | NA | NA | NA | NA |
| DDHD2 | NA | NA | NA | NA | NA |
| DDI2 | NA | 447.862 | −6.822783 | uc001aww.3 | chr1: 15960065- |
| | | | | | 15960796 |
| DDN | chr12: 49388933- | NA | NA | NA | NA |
| | 49390522 | | | | |
| DDOST | chr1: 20978260- | NA | NA | NA | NA |
| | 20978871 | | | | |
| DDRGK1 | chr20: 3171012- | NA | NA | NA | NA |
| | 3171298 | | | | |
| DDT | NA | 675.886 | −11.182104 | uc002zyz.4 | chr22: 24313554- |
| | | | | | 24313739 |
| DDX17 | NA | NA | NA | NA | NA |
| DDX18 | NA | 686.938 | −7.224684 | uc002tlh.1 | chr2: 118588301- |
| | | | | | 118589953 |
| DDX31 | NA | 690.491 | −4.410208 | uc004cbq.1 | chr9: 135469676- |
| | | | | | 135470252 |
| DDX3Y | chrY: 15026580- | NA | NA | NA | NA |
| | 15026743 | | | | |
| DDX4 | NA | NA | NA | NA | NA |
| DDX47 | chr12: 12982489- | NA | NA | NA | NA |
| | 12982915 | | | | |
| DDX5 | NA | NA | NA | NA | NA |
| DDX52 | NA | NA | NA | NA | NA |
| DDX55 | chr12: 124104021- | NA | NA | NA | NA |
| | 124104255 | | | | |
| DDX59 | chr1: 200610119- | NA | NA | NA | NA |
| | 200610276 | | | | |
| DDX6 | NA | NA | NA | NA | NA |
| DECR1 | chr8: 91064126- | NA | NA | NA | NA |
| | 91064227 | | | | |
| DEFA6 | NA | NA | NA | NA | NA |
| DEFB129 | chr20: 210413- | NA | NA | NA | NA |
| | 210527 | | | | |
| DEGS2 | NA | 594.775 | −9.068463 | uc001ygx.2 | chr14: 100612753- |
| | | | | | 100613097 |
| DENND1B | NA | 765.802 | −4.807437 | uc021pgu.1 | chr1: 197473879- |
| | | | | | 197479589 |
| DENND2C | NA | 578.519 | −8.747386 | uc001efc.1 | chr1: 115127196- |
| | | | | | 115128220 |
| DENND4A | chr15: 66007413- | NA | NA | NA | NA |
| | 66007785 | | | | |
| DENND4C | NA | 288.004 | −5.459773 | uc003znr.3 | chr9: 19369990- |
| | | | | | 19374137 |
| DENND5B | NA | 436.577 | −5.574706 | uc001rkh.1 | chr12: 31535157- |
| | | | | | 31540536 |
| DEPDC1B | NA | 343.008 | −7.070358 | uc003jsh.3 | chr5: 59892739- |
| | | | | | 59893579 |
| DEPDC5 | chr22: 32302484- | NA | NA | NA | NA |
| | 32303020 | | | | |
| DEPDC7 | NA | NA | NA | NA | NA |
| DERA | NA | 235.613 | −4.589976 | uc001rde.3 | chr12: 16189681- |
| | | | | | 16190315 |
| DERL2 | chr17: 5377613- | NA | NA | NA | NA |
| | 5378003 | | | | |
| DFFA | chr1: 10520603- | 702.658 | −3.637846 | uc001arj.3 | chr1: 10520603- |
| | 10521546 | | | | 10521546 |
| DFFB | chr1: 3782868- | NA | NA | NA | NA |
| | 3782962 | | | | |
| DGAT2 | NA | 305.988 | −5.309166 | uc001oxa.3 | chr11: 75511554- |
| | | | | | 75512581 |
| DQCR6 | NA | NA | NA | NA | NA |
| DGKB | NA | NA | NA | NA | NA |
| DGKH | chr13: 42803288- | 894.731 | −7.136125 | uc010tfh.2 | chr13: 42803288- |
| | 42803891 | | | | 42803891 |
| DHFRL1 | NA | NA | NA | NA | NA |
| DHPS | NA | 879.604 | −6.756444 | NA | NA |
| DHRS1 | NA | NA | NA | NA | NA |
| DHRS11 | chr17: 34956633- | NA | NA | NA | NA |
| | 34957233 | | | | |
| DHRS7 | chr14: 60611500- | 388.341 | −4.578215 | uc001xes.3 | chr14: 60611500- |
| | 60611683 | | | | 60611683 |
| DHRS7B | chr17: 21094467- | NA | NA | NA | NA |
| | 21095285 | | | | |
| DHX30 | NA | 571.196 | −4.79969 | uc003crs.2 | chr3: 47883246- |
| | | | | | 47884169 |
| DHX32 | chr10: 127524909- | NA | NA | NA | NA |
| | 127525255 | | | | |
| DHX33 | NA | NA | NA | NA | NA |
| DHX35 | NA | NA | NA | NA | NA |
| DHX40 | NA | NA | NA | NA | NA |
| DHX57 | NA | 1809.4 | −14.814332 | uc002rrd.4 | chr2: 39024876- |
| | | | | | 39025440 |
| DHX9 | chr1: 182856570- | NA | NA | NA | NA |
| | 182857117 | | | | |
| DICER1 | NA | NA | NA | NA | NA |
| DIEXF | NA | NA | NA | NA | NA |
| DIMT1 | NA | NA | NA | NA | NA |
| DIO1 | NA | 260.92 | −5.589298 | uc001cwb.3 | chr1: 54375673- |
| | | | | | 54376759 |
| DIO2 | chr14: 80663868- | NA | NA | NA | NA |
| | 80669631 | | | | |
| DIP2A | chr21: 47966960- | 623.185 | −5.761203 | uc002zjn.3 | chr21: 47969832- |
| | 47967120 | | | | 47970023 |
| DIP2C | chr10: 320130- | 793.04 | 2.480598 | uc009xhi.1 | chr10: 386420- |
| | 323264 | | | | 386610 |
| DIRAS3 | chr1: 68511645- | NA | NA | NA | NA |
| | 68512290 | | | | |
| DIRC1 | chr2: 189598465- | 699.826 | −10.800752 | uc002uqi.1 | chr2: 189598465- |
| | 189599332 | | | | 189599332 |
| DIS3L | NA | 739.829 | −7.0895 | NA | NA |
| DIXDC1 | NA | NA | NA | NA | NA |
| DKFZP434K028 | NA | 300.509 | −6.604706 | uc001nsd.3 | chr11: 61521501- |
| | | | | | 61521917 |
| DLC1 | NA | 849.881 | −1.712748 | uc011kxx.1 | chr8: 12940872- |
| | | | | | 12943319 |
| DLD | chr7: 107559705- | NA | NA | NA | NA |
| | 107561643 | | | | |
| DLEU7 | NA | 700.763 | −4.889924 | uc001vez.3 | chr13: 51397046- |
| | | | | | 51397449 |
| DLG1 | NA | 483.081 | −5.764825 | uc0Ubub.2 | chr3: 196769431- |
| | | | | | 196771493 |
| DLG2 | chr11: 83166056- | 1054.69 | −7.651467 | uc010rtb.1 | chr11: 83172585- |
| | 83170860 | | | | 83173015 |
| DLG4 | NA | NA | NA | NA | NA |
| DLG5 | chr10: 79575791- | NA | NA | NA | NA |
| | 79575929 | | | | |
| DLGAP5 | NA | 1085.46 | −6.57252 | uc001xbs.3 | chr14: 55614834- |
| | | | | | 55615068 |
| DLK1 | NA | 1446.84 | −13.401035 | uc001yhu.4 | chr14: 101201234- |
| | | | | | 101201464 |
| DLK2 | chr6: 43418090- | NA | NA | NA | NA |
| | 43418276 | | | | |
| DLX2 | NA | 220.19 | −4.299868 | uc010zdx.1 | chr2: 172965873- |
| | | | | | 172966120 |
| DLX5 | chr7: 96649702- | 1163.95 | −4.84583 | uc003uon.3 | chr7: 96649702- |
| | 96650047 | | | | 96650047 |
| DMAP1 | chr1: 44680996- | NA | NA | NA | NA |
| | 44681440 | | | | |
| DMBT1 | NA | NA | NA | NA | NA |
| DMD | chrX: 31137345- | NA | NA | NA | NA |
| | 31139949 | | | | |
| DMPK | chr19: 46272976- | NA | NA | NA | NA |
| | 46273738 | | | | |
| DMRT1 | NA | NA | NA | NA | NA |
| DMRTA1 | NA | 375.175 | −9.825844 | uc003zpp.1 | chr9: 22451911- |
| | | | | | 22452472 |
| DMRTA2 | chr1: 50883223- | NA | NA | NA | NA |
| | 50884336 | | | | |
| DMRTB1 | chr1: 53932336- | 316.958 | −3.730877 | uc001cvq.1 | chr1: 53932336- |
| | 53933158 | | | | 53933158 |
| DMXL1 | chr5: 118582915- | NA | NA | NA | NA |
| | 118584822 | | | | |
| DMXL2 | chr15: 51739921- | 676.628 | −6.448375 | uc002abd.3 | chr15: 51739921- |
| | 51741180 | | | | 51741180 |
| DNER | NA | 514.661 | −7.514009 | uc002vpv.3 | chr2: 230222345- |
| | | | | | 230223255 |
| DNHD1 | chr11: 6541342- | NA | NA | NA | NA |
| | 6542097 | | | | |
| DNM2 | NA | 237.626 | −6.105755 | uc002mpx.1 | chr19: 10943020- |
| | | | | | 10944160 |
| DNM3 | NA | 878.73 | −10.110392 | uc001gih.1 | chr1: 172387458- |
| | | | | | 172387567 |
| DNMT3A | NA | NA | NA | NA | NA |
| DNTT | chr10: 98097982- | 579.112 | −8.124877 | uc001kmf.3 | chr10: 98097982- |
| | 98098321 | | | | 98098321 |
| DNTTIP1 | chr20: 44439835- | NA | NA | NA | NA |
| | 44440066 | | | | |
| DNTTIP2 | chr1: 94335014- | NA | NA | NA | NA |
| | 94335406 | | | | |
| DOCK10 | NA | 1021.17 | −10.524106 | uc002vod.1 | chr2: 225725297- |
| | | | | | 225727283 |
| DOCK4 | NA | 219.901 | −5.20242 | uc003vfv.3 | chr7: 111366164- |
| | | | | | 111368329 |
| DOCK7 | chr1: 62920397- | NA | NA | NA | NA |
| | 62921084 | | | | |
| DOK2 | NA | 434.907 | −5.256292 | uc003wzy.1 | chr8: 21766384- |
| | | | | | 21766821 |
| DOK7 | NA | NA | NA | NA | NA |
| DOPEY2 | chr21: 37633035- | NA | NA | NA | NA |
| | 37634210 | | | | |
| DPAGT1 | NA | 818.874 | −10.795756 | uc001pvi.3 | chr11: 118967213- |
| | | | | | 118967707 |
| DPF3 | chr14: 73136660- | NA | NA | NA | NA |
| | 73137843 | | | | |
| DPH6 | NA | 972.535 | −6.417472 | uc001zja.3 | chr15: 35663170- |
| | | | | | 35664350 |
| DPM1 | chr20: 49551405- | 785.505 | −7.808491 | uc002xvw.1 | chr20: 49551405- |
| | 49551668 | | | | 49551668 |
| DPP8 | chr15: 65737998- | NA | NA | NA | NA |
| | 65739221 | | | | |
| DPPA3 | NA | NA | NA | NA | NA |
| DPT | NA | NA | NA | NA | NA |
| DPY19L1 | chr7: 34968493- | NA | NA | NA | NA |
| | 34971184 | | | | |
| DPY19L2 | chr12: 63952693- | 362.334 | −5.856552 | uc010sso.1 | chr12: 63952693- |
| | 63954291 | | | | 63954291 |
| DPY19L2P2 | chr7: 102815462- | 296.076 | −7.177071 | uc003vbi.2 | chr7: 102913539- |
| | 102815837 | | | | 102916050 |
| DPY19L4 | NA | NA | NA | NA | NA |
| DR1 | chr1: 93826197- | NA | NA | NA | NA |
| | 93828148 | | | | |
| DROSHA | NA | 861.79 | −6.159437 | uc003jhg.2 | chr5: 31400602- |
| | | | | | 31401538 |
| DSC3 | chr18: 28570052- | NA | NA | NA | NA |
| | 28574338 | | | | |
| DSCAM | chr21: 41384343- | NA | NA | NA | NA |
| | 41384960 | | | | |
| DSCR3 | chr21: 38595726- | NA | NA | NA | NA |
| | 38597844 | | | | |
| DSE | NA | 923.361 | −10.160625 | uc003pwq.1 | chr6: 116720834- |
| | | | | | 116722435 |
| DSG1 | NA | 590.098 | −11.736124 | uc002kwp.3 | chr18: 28935310- |
| | | | | | 28937393 |
| DSG3 | chr18: 29056224- | NA | NA | NA | NA |
| | 29058665 | | | | |
| DSG4 | chr18: 28993559- | 852.304 | −11.248012 | uc002kwq.2 | chr18: 28993559- |
| | 28993880 | | | | 28993880 |
| DSP | NA | 342.776 | −9.238745 | uc003mxp.1 | chr6: 7586112- |
| | | | | | 7586946 |
| DST | chr6: 56348351- | NA | NA | NA | NA |
| | 56350031 | | | | |
| DSTN | NA | NA | NA | NA | NA |
| DTD2 | chr14: 31915243- | NA | NA | NA | NA |
| | 31917334 | | | | |
| DTNBP1 | NA | 335.803 | −8.312605 | uc003nbl.3 | chr6: 15523032- |
| | | | | | 15523205 |
| DTWD1 | NA | 1034.07 | −12.15245 | uc001zxq.3 | chr15: 49935776- |
| | | | | | 49937333 |
| DTX2 | chr7: 76134919- | 638.704 | −5.628727 | uc011kgk.1 | chr7: 76135224- |
| | 76135312 | | | | 76135305 |
| DUSP10 | chr1: 221874764- | NA | NA | NA | NA |
| | 221875753 | | | | |
| DUSP12 | NA | 523.22 | −6.98762 | uc001gbo.3 | chr1: 161726738- |
| | | | | | 161726952 |
| DUSP16 | NA | 1487.99 | −11.356718 | uc001ran.2 | chr12: 12626216- |
| | | | | | 12629766 |
| DUSP18 | NA | 970.61 | −7.706717 | uc003aiu.3 | chr22: 31048038- |
| | | | | | 31048284 |
| DUSP19 | chr2: 183960387- | NA | NA | NA | NA |
| | 183964722 | | | | |
| DUSP22 | NA | NA | NA | NA | NA |
| DUSP28 | chr2: 241503091- | NA | NA | NA | NA |
| | 241503431 | | | | |
| DUSP5 | NA | NA | NA | NA | NA |
| DUSP5P1 | chr1: 228786372- | NA | NA | NA | NA |
| | 228788159 | | | | |
| DUSP6 | chr12: 89741837- | NA | NA | NA | NA |
| | 89743030 | | | | |
| DUSP8 | chr11: 1575281- | 494.151 | −8.799424 | uc001lts.2 | chr11: 1575281- |
| | 1577747 | | | | 1577747 |
| DUT | NA | NA | NA | NA | NA |
| DUXA | NA | NA | NA | NA | NA |
| DVL3 | chr3: 183888544- | 431.247 | −4.620109 | uc003fms.3 | chr3: 183888544- |
| | 183891314 | | | | 183891314 |
| DYM | chr18: 46570172- | 1272.42 | −2.520133 | uc002ldi.1 | chr18: 46570172- |
| | 46570424 | | | | 46570424 |
| DYNC1H1 | NA | NA | NA | NA | NA |
| DYNC1LI1 | NA | NA | NA | NA | NA |
| DYNC2LI1 | chr2: 44019930- | 837.569 | −5.844674 | uc002rtk.3 | chr2: 44036907- |
| | 44022322 | | | | 44037149 |
| DYNLT1 | NA | NA | NA | NA | NA |
| DYRK1A | chr21: 38884214- | 606.627 | −4.549532 | uc002ywi.3 | chr21: 38878611- |
| | 38887679 | | | | 38878657 |
| DYRK3 | chr1: 206822311- | 861.737 | −8.641795 | uc001hei.3 | chr1: 206822311- |
| | 206822542 | | | | 206822542 |
| DYRK4 | NA | 217.382 | −8.23953 | uc001qmx.3 | chr12: 4722920- |
| | | | | | 4723041 |
| DYTN | NA | 486.423 | −6.615056 | uc002vbr.1 | chr2: 207516345- |
| | | | | | 207516541 |
| DZIP1 | NA | NA | NA | NA | NA |
| DZIP3 | chr3: 108409745- | 316.973 | −6.597742 | uc003dxd.3 | chr3: 108412207- |
| | 108410234 | | | | 108413693 |
| E2F5 | NA | NA | NA | NA | NA |
| EAF1 | chr3: 15480663- | NA | NA | NA | NA |
| | 15484120 | | | | |
| EAPP | NA | NA | NA | NA | NA |
| EARS2 | chr16: 23533334- | NA | NA | NA | NA |
| | 23535691 | | | | |
| EBF2 | NA | 233.179 | −5.057655 | uc003xes.2 | chr8: 25699246- |
| | | | | | 25702124 |
| EBI3 | chr19: 4237086- | NA | NA | NA | NA |
| | 4237524 | | | | |
| EBLN1 | NA | NA | NA | NA | NA |
| EBPL | NA | NA | NA | NA | NA |
| ECM2 | NA | 2505.86 | −20.421797 | uc004asf.4 | chr9: 95255829- |
| | | | | | 95256364 |
| ECSIT | chr19: 11616731- | NA | NA | NA | NA |
| | 11617243 | | | | |
| EDA2R | chrX: 65815482- | NA | NA | NA | NA |
| | 65817935 | | | | |
| EDC3 | NA | NA | NA | NA | NA |
| EDC4 | NA | 569.887 | −6.190109 | uc002eur.3 | chr16: 67918052- |
| | | | | | 67918417 |
| EDN1 | chr6: 12292747- | NA | NA | NA | NA |
| | 12293113 | | | | |
| EDN3 | NA | NA | NA | NA | NA |
| EDNRB | chr13: 78469616- | 923.921 | −8.589803 | uc001vko.2 | chr13: 78469616- |
| | 78472334 | | | | 78472334 |
| EDRF1 | chr10: 127436624- | 538.29 | −5.622688 | uc001lio.1 | chr10: 127452042- |
| | 127437652 | | | | 127452712 |
| EFCAB11 | chr14: 90263469- | NA | NA | NA | NA |
| | 90263586 | | | | |
| EFCAB14 | chr1: 47140831- | NA | NA | NA | NA |
| | 47144132 | | | | |
| EFCAB3 | chr17: 60493691- | NA | NA | NA | NA |
| | 60493839 | | | | |
| EFCAB5 | NA | 1406.13 | −14.753148 | uc002het.3 | chr17: 28435043- |
| | | | | | 28435470 |
| EFCAB7 | NA | NA | NA | NA | NA |
| EFHB | NA | NA | NA | NA | NA |
| EFHC2 | chrX: 44007128- | NA | NA | NA | NA |
| | 44008040 | | | | |
| EFNB1 | chrX: 68060498- | NA | NA | NA | NA |
| | 68062006 | | | | |
| EFNB2 | NA | NA | NA | NA | NA |
| EFNB3 | chr17: 7612895- | NA | NA | NA | NA |
| | 7614693 | | | | |
| EFTUD1 | chr15: 82422561- | 996.066 | −12.502846 | uc002bgt.1 | chr15: 82422561- |
| | 82422713 | | | | 82422713 |
| EFTUD2 | NA | 377.487 | −5.15376 | uc002ihn.2 | chr17: 42927655- |
| | | | | | 42928641 |
| EGFEM1P | NA | NA | NA | NA | NA |
| EGFL6 | NA | 495.428 | −9.285163 | uc004cvi.3 | chrX: 13651215- |
| | | | | | 13651694 |
| EGLN1 | chr1: 231499497- | 358.577 | −5.690497 | uc001huu.3 | chr1: 231499497- |
| | 231502156 | | | | 231502156 |
| EHBP1 | chr2: 63053408- | 532.65 | −15.484832 | uc010fcp.3 | chr2: 63272636- |
| | 63053716 | | | | 63273621 |
| EHD3 | NA | 435.874 | −8.031125 | uc002rnu.3 | chr2: 31489571- |
| | | | | | 31491260 |
| EHD4 | NA | NA | NA | NA | NA |
| EID1 | NA | NA | NA | NA | NA |
| EID2 | chr19: 40029447- | NA | NA | NA | NA |
| | 40030008 | | | | |
| EID3 | NA | NA | NA | NA | NA |
| EIF1AD | NA | NA | NA | NA | NA |
| EIF1AX | chrX: 20142636- | NA | NA | NA | NA |
| | 20146423 | | | | |
| EIF1AY | chrY: 22754233- | NA | NA | NA | NA |
| | 22755040 | | | | |
| EIF2AK4 | chr15: 40265984- | 541.583 | −5.063079 | uc001zkl.3 | chr15: 40265984- |
| | 40267625 | | | | 40267625 |
| EIF2B4 | NA | 521.906 | −8.508304 | uc002rkf.1 | chr2: 27589089- |
| | | | | | 27591481 |
| EIF2D | chr1: 206764974- | 866.722 | −8.29099 | uc001heh.2 | chr1: 206764974- |
| | 206765106 | | | | 206765106 |
| EIF2S1 | NA | 205.219 | −4.919721 | uc001xjg.3 | chr14: 67850158- |
| | | | | | 67853233 |
| EIF2S2 | chr20: 32676115- | NA | NA | NA | NA |
| | 32677535 | | | | |
| EIF2S3 | chrX: 24094903- | NA | NA | NA | NA |
| | 24096927 | | | | |
| EIF3G | NA | NA | NA | NA | NA |
| EIF3H | chr8: 117670488- | 1328.18 | −11.143041 | uc011lhz.1 | chr8: 117670488- |
| | 117671046 | | | | 117671046 |
| EIF3IP1 | NA | NA | NA | NA | NA |
| EIF3J | chr15: 44853348- | 1212.39 | −8.965148 | uc001ztv.3 | chr15: 44853348- |
| | 44855001 | | | | 44855001 |
| EIF3L | NA | 358.478 | −4.884203 | uc003auf.3 | chr22: 38284472- |
| | | | | | 38284789 |
| EIF4A2 | NA | 260.807 | −6.483717 | uc003fqs.3 | chr3: 186507059- |
| | | | | | 186507685 |
| EIF4A3 | chr17: 78109013- | NA | NA | NA | NA |
| | 78109288 | | | | |
| EIF4B | NA | 501.382 | −4.879666 | uc001sbh.4 | chr12: 53434008- |
| | | | | | 53435993 |
| EIF4E | NA | 443.651 | −5.549346 | uc011cea.1 | chr4: 99799607- |
| | | | | | 99802178 |
| EIF4E2 | NA | 1079.82 | −11.646689 | uc002vtb.1 | chr2: 233445654- |
| | | | | | 233448349 |
| EIF4EBP2 | chr10: 72181504- | NA | NA | NA | NA |
| | 72188374 | | | | |
| EIF5AL1 | NA | NA | NA | NA | NA |
| ELAVL4 | NA | 386.873 | −5.219938 | uc009vyu.3 | chr1: 50663144- |
| | | | | | 50663232 |
| ELF2 | NA | NA | NA | NA | NA |
| ELF5 | NA | 626.728 | −6.99685 | uc001mvq.2 | chr11: 34513144- |
| | | | | | 34514963 |
| ELK4 | NA | 1024.36 | −8.317603 | uc001hcz.3 | chr1: 205588396- |
| | | | | | 205588955 |
| ELL3 | NA | 330.213 | −4.568145 | uc001zsv.1 | chr15: 44064798- |
| | | | | | 44065313 |
| ELMOD1 | NA | NA | NA | NA | NA |
| ELMOD2 | NA | 1180.61 | −10.152742 | uc003iik.3 | chr4: 141471530- |
| | | | | | 141474924 |
| ELN | chr7: 73483031- | NA | NA | NA | NA |
| | 73484236 | | | | |
| ELOVL7 | chr5: 60047616- | NA | NA | NA | NA |
| | 60050450 | | | | |
| ELP2 | chr18: 33754605- | NA | NA | NA | NA |
| | 33754688 | | | | |
| ELP4 | NA | NA | NA | NA | NA |
| ELSPBP1 | chr19: 48528209- | NA | NA | NA | NA |
| | 48528410 | | | | |
| EMBP1 | chr1: 121310592- | NA | NA | NA | NA |
| | 121313686 | | | | |
| EMC10 | NA | NA | NA | NA | NA |
| EMC2 | NA | NA | NA | NA | NA |
| EMC3 | NA | NA | NA | NA | NA |
| EMC4 | chr15: 34521990- | 758.816 | −5.248158 | uc001zhq.3 | chr15: 34521990- |
| | 34522352 | | | | 34522352 |
| EMCN | NA | 200.533 | −4.275788 | uc003hvr.3 | chr4: 101316498- |
| | | | | | 101319530 |
| EMILIN2 | NA | NA | NA | NA | NA |
| EML1 | NA | 813.741 | −9.802606 | uc010tww.2 | chr14: 100406450- |
| | | | | | 100408395 |
| EML4 | NA | 233.012 | −4.416075 | uc002rsi.3 | chr2: 42557348- |
| | | | | | 42559688 |
| EMP2 | NA | NA | NA | NA | NA |
| ENDOD1 | chr11: 94862744- | NA | NA | NA | NA |
| | 94865815 | | | | |
| ENDOV | NA | 682.555 | −11.897688 | uc002jyj.1 | chr17: 78393850- |
| | | | | | 78394333 |
| ENGASE | NA | NA | NA | NA | NA |
| ENO2 | chr12: 7031964- | 362.928 | −5.945776 | uc001qru.1 | chr12: 7031964- |
| | 7032859 | | | | 7032859 |
| ENOPH1 | chr4: 83381274- | NA | NA | NA | NA |
| | 83382244 | | | | |
| ENPEP | NA | NA | NA | NA | NA |
| ENPP2 | chr8: 120569317- | NA | NA | NA | NA |
| | 120569760 | | | | |
| ENPP3 | NA | NA | NA | NA | NA |
| ENPP4 | NA | NA | NA | NA | NA |
| ENSA | chr1: 150595754- | NA | NA | NA | NA |
| | 150598113 | | | | |
| ENTHD1 | NA | NA | NA | NA | NA |
| ENTPD1 | NA | 517.392 | −8.299582 | uc001kle.1 | chr10: 97605441- |
| | | | | | 97605843 |
| ENTPD4 | NA | NA | NA | NA | NA |
| ENY2 | chr8: 110355711- | NA | NA | NA | NA |
| | 110358189 | | | | |
| EOGT | NA | 447.67 | −7.564841 | uc003dnj.3 | chr3: 69024368- |
| | | | | | 69026768 |
| EP300 | NA | NA | NA | NA | NA |
| EP400NL | chr12: 132593300- | NA | NA | NA | NA |
| | 132593444 | | | | |
| EPB41L1 | chr20: 34817263- | NA | NA | NA | NA |
| | 34820721 | | | | |
| EPB41L4B | chr9: 111934254- | NA | NA | NA | NA |
| | 111936833 | | | | |
| EPB41L5 | NA | 755.1 | −4.183678 | uc010flk.3 | chr2: 120861817- |
| | | | | | 120864492 |
| EPDR1 | NA | NA | NA | NA | NA |
| EPG5 | chr18: 43487830- | 585.057 | −11.361198 | uc002lbm.3 | chr18: 43427574- |
| | 43487910 | | | | 43432431 |
| EPHA2 | chr1: 16450832- | NA | NA | NA | NA |
| | 16451709 | | | | |
| EPHA3 | NA | 609.209 | −6.762516 | uc003dqx.1 | chr3: 89448657- |
| | | | | | 89449495 |
| EPHA4 | chr2: 222289929- | NA | NA | NA | NA |
| | 222290747 | | | | |
| EPHA5 | NA | 403.336 | −5.268298 | uc003hda.2 | chr4: 66196279- |
| | | | | | 66197686 |
| EPHA7 | chr6: 93949740- | NA | NA | NA | NA |
| | 93953143 | | | | |
| EPHX3 | chr19: 15337730- | NA | NA | NA | NA |
| | 15338247 | | | | |
| EPHX4 | NA | NA | NA | NA | NA |
| EPM2A | NA | 777.622 | −6.481523 | uc010khr.3 | chr6: 145946440- |
| | | | | | 145948630 |
| EPN2 | NA | NA | NA | NA | NA |
| EPPK1 | NA | 1584.25 | −16.369205 | uc003zaa.1 | chr8: 144939912- |
| | | | | | 144940158 |
| EPRS | NA | 328.01 | −6.758649 | uc001hly.1 | chr1: 220141942- |
| | | | | | 220142147 |
| EPS15 | NA | NA | NA | NA | NA |
| EPS8 | NA | NA | NA | NA | NA |
| EPS8L2 | NA | NA | NA | NA | NA |
| EPYC | NA | NA | NA | NA | NA |
| ERAP1 | chr5: 96096514- | NA | NA | NA | NA |
| | 96098903 | | | | |
| ERBB4 | NA | NA | NA | NA | NA |
| ERC1 | chr12: 1353793- | NA | NA | NA | NA |
| | 1354122 | | | | |
| ERCC3 | chr2: 128047272- | NA | NA | NA | NA |
| | 128047809 | | | | |
| ERCC4 | chr16: 14026160- | NA | NA | NA | NA |
| | 14027104 | | | | |
| ERCC6L | chrX: 71424507- | NA | NA | NA | NA |
| | 71424863 | | | | |
| ERCC8 | chr5: 60199187- | 468.567 | −7.049464 | uc003jsl.3 | chr5: 60169659- |
| | 60199406 | | | | 60170441 |
| EREG | NA | NA | NA | NA | NA |
| ERGIC3 | chr20: 34144130- | NA | NA | NA | NA |
| | 34144209 | | | | |
| ERI1 | NA | NA | NA | NA | NA |
| ERI2 | chr16: 20807662- | NA | NA | NA | NA |
| | 20809045 | | | | |
| ERICH3 | chr1: 75055011- | NA | NA | NA | NA |
| | 75055297 | | | | |
| ERMAP | chr1: 43304655- | NA | NA | NA | NA |
| | 43305384 | | | | |
| ERMN | chr2: 158175125- | NA | NA | NA | NA |
| | 158177782 | | | | |
| ERP29 | NA | 350.937 | −5.61809 | uc001ttk.1 | chr12: 112460457- |
| | | | | | 112461024 |
| ERP44 | NA | NA | NA | NA | NA |
| ESCO2 | chr8: 27660956- | NA | NA | NA | NA |
| | 27662424 | | | | |
| ESF1 | chr20: 13694969- | NA | NA | NA | NA |
| | 13695520 | | | | |
| ESPL1 | NA | 490.643 | −3.832978 | uc001scj.2 | chr12: 53687259- |
| | | | | | 53687427 |
| ESRRA | NA | 303.868 | −4.856061 | uc001nzq.1 | chr11: 64083439- |
| | | | | | 64084210 |
| ESX1 | NA | NA | NA | NA | NA |
| ESYT2 | NA | NA | NA | NA | NA |
| ETFB | chr19: 51848409- | NA | NA | NA | NA |
| | 51848464 | | | | |
| ETNK1 | NA | 596.548 | −4.659142 | uc001rft.3 | chr12: 22837889- |
| | | | | | 22843608 |
| ETV1 | chr7: 13930856- | NA | NA | NA | NA |
| | 13935490 | | | | |
| ETV5 | NA | NA | NA | NA | NA |
| EVC2 | chr4: 5564146- | NA | NA | NA | NA |
| | 5564574 | | | | |
| EVI5L | NA | 243.854 | −3.885436 | uc002mio.1 | chr19: 7921871- |
| | | | | | 7924076 |
| EVPL | chr17: 74002927- | NA | NA | NA | NA |
| | 74003183 | | | | |
| EXOC4 | NA | 977.208 | −12.986352 | uc003vrk.3 | chr7: 133749282- |
| | | | | | 133750513 |
| EXOC6 | NA | NA | NA | NA | NA |
| EXOC7 | chr17: 74077086- | 618.563 | −3.988751 | uc010dgv.2 | chr17: 74077086- |
| | 74079757 | | | | 74079728 |
| EXOC8 | NA | 1116.54 | −6.218359 | uc001huq.3 | chr1: 231468482- |
| | | | | | 231471313 |
| EXOSC10 | chr1: 11139768- | 976.105 | −8.322627 | uc001asa.3 | chr1: 11126676- |
| | 11140816 | | | | 11126774 |
| EXOSC8 | chr13: 37583437- | NA | NA | NA | NA |
| | 37583751 | | | | |
| EXOSC9 | chr4: 122738012- | NA | NA | NA | NA |
| | 122738176 | | | | |
| EYA4 | NA | 841.256 | −3.579343 | uc011ecq.2 | chr6: 133849944- |
| | | | | | 133853258 |
| EZH1 | chr17: 40852293- | 766.538 | −0.41409 | uc002iaz.3 | chr17: 40852293- |
| | 40854549 | | | | 40854549 |
| EZH2 | chr7: 148504464- | NA | NA | NA | NA |
| | 148504737 | | | | |
| EZR | chr6: 159186773- | NA | NA | NA | NA |
| | 159187945 | | | | |
| F11R | chr1: 160965001- | 360.423 | −8.223133 | uc010pjv.2 | chr1: 160965001- |
| | 160968660 | | | | 160968660 |
| FAAH2 | NA | 722.032 | −16.297911 | uc004dvc.3 | chrX: 57515366- |
| | | | | | 57515629 |
| FABP1 | NA | 243.513 | −12.171129 | uc002sst.2 | chr2: 88422508- |
| | | | | | 88422572 |
| FABP4 | NA | 1685 | −10.218914 | uc003ycd.2 | chr8: 82390732- |
| | | | | | 82391099 |
| FABP7 | chr6: 123102493- | NA | NA | NA | NA |
| | 123104039 | | | | |
| FADD | NA | NA | NA | NA | NA |
| FAF2 | chr5: 175933952- | 575.315 | −6.645377 | uc003mej.4 | chr5: 175933952- |
| | 175937075 | | | | 175937075 |
| FAHD1 | chr16: 1877906- | 272.608 | −9.451525 | uc002cnc.1 | chr16: 1877906- |
| | 1878908 | | | | 1878908 |
| FAM104A | NA | NA | NA | NA | NA |
| FAM110B | chr8: 59059903- | NA | NA | NA | NA |
| | 59062277 | | | | |
| FAM111A | NA | 534.164 | −5.315011 | uc001nno.3 | chr11: 58920978- |
| | | | | | 58922511 |
| FAM117B | chr2: 203589757- | 668.866 | −5.604784 | uc010zhx.2 | chr2: 203630488- |
| | 203589955 | | | | 203634480 |
| FAM120AOS | NA | NA | NA | NA | NA |
| FAM122B | chrX: 133903596- | NA | NA | NA | NA |
| | 133905314 | | | | |
| FAM126A | NA | 275.4 | −7.84276 | uc003svm.4 | chr7: 22980878- |
| | | | | | 22985207 |
| FAM126B | NA | 583.968 | −12.737066 | uc002uws.4 | chr2: 201838441- |
| | | | | | 201845992 |
| FAM134C | NA | NA | NA | NA | NA |
| FAM135A | chr6: 71269625- | 687.071 | −2.933073 | uc003pfk.3 | chr6: 71233866- |
| | 71270877 | | | | 71234306 |
| FAM135B | chr8: 139155103- | NA | NA | NA | NA |
| | 139155250 | | | | |
| FAM13B | chr5: 137273642- | NA | NA | NA | NA |
| | 137275913 | | | | |
| FAM13C | NA | 458.457 | −7.298557 | uc010qif.1 | chr10: 61010929- |
| | | | | | 61011246 |
| FAM149A | chr4: 187079202- | NA | NA | NA | NA |
| | 187079564 | | | | |
| FAM149B1 | chr10: 75000778- | NA | NA | NA | NA |
| | 75001939 | | | | |
| FAM151B | chr5: 79837652- | 748.131 | −8.338082 | uc003kgv.2 | chr5: 79837652- |
| | 79838382 | | | | 79838382 |
| FAM153C | NA | 499.572 | −5.804305 | uc011dge.2 | chr5: 177475666- |
| | | | | | 177476088 |
| FAM160A1 | NA | NA | NA | NA | NA |
| FAM160A2 | NA | 506.453 | −5.384782 | uc001mck.4 | chr11: 6232564- |
| | | | | | 6232735 |
| FAM162A | chr3: 122126291- | NA | NA | NA | NA |
| | 122126811 | | | | |
| FAM163A | NA | 815.154 | −6.686591 | uc009wxj.3 | chr1: 179783325- |
| | | | | | 179785333 |
| FAM166A | chr9: 140138037- | NA | NA | NA | NA |
| | 140138151 | | | | |
| FAM166B | chr9: 35561827- | NA | NA | NA | NA |
| | 35562003 | | | | |
| FAM168B | NA | 775.6 | −4.023616 | uc002tsd.3 | chr2: 131805449- |
| | | | | | 131810025 |
| FAM169A | chr5: 74073399- | NA | NA | NA | NA |
| | 74077284 | | | | |
| FAM171B | chr2: 187627551- | 721.276 | −7.390355 | uc002upr.1 | chr2: 187627551- |
| | 187628335 | | | | 187628335 |
| FAM172A | chr5: 92953431- | NA | NA | NA | NA |
| | 92956692 | | | | |
| FAM172BP | chr3: 101241824- | NA | NA | NA | NA |
| | 101242731 | | | | |
| FAM175A | chr4: 84382094- | 1832.87 | −11.049168 | uc003hou.2 | chr4: 84382094- |
| | 84383621 | | | | 84383621 |
| FAM177A1 | NA | NA | NA | NA | NA |
| FAM177B | chr1: 222923401- | NA | NA | NA | NA |
| | 222924002 | | | | |
| FAM179A | chr2: 29268405- | 1065.95 | −11.30611 | uc010ezl.3 | chr2: 29274960- |
| | 29270413 | | | | 29275096 |
| FAM179B | chr14: 45475492- | NA | NA | NA | NA |
| | 45477346 | | | | |
| FAM180A | NA | NA | NA | NA | NA |
| FAM184A | chr6: 119280996- | NA | NA | NA | NA |
| | 119281267 | | | | |
| FAM189A2 | chr9: 72006675- | 666.229 | −6.158318 | uc010mon.1 | chr9: 72006721- |
| | 72007370 | | | | 72007370 |
| FAM189B | chr1: 155222540- | 1247.63 | −8.866505 | uc001fjq.1 | chr1: 15522254O- |
| | 155223343 | | | | 155223343 |
| FAM193A | NA | 378.919 | −4.387198 | uc010ick.3 | chr4: 2733596- |
| | | | | | 2734302 |
| FAM198A | NA | NA | NA | NA | NA |
| FAM198B | NA | NA | NA | NA | NA |
| FAM199X | NA | 734.102 | −12.254555 | uc004elw.3 | chrX: 103434460- |
| | | | | | 103440582 |
| FAM200A | NA | NA | NA | NA | NA |
| FAM208A | chr3: 56654160- | 2323.21 | −12.721424 | uc003dib.4 | chr3: 56654160- |
| | 56657064 | | | | 56657064 |
| FAM20B | chr1: 179041280- | NA | NA | NA | NA |
| | 179045702 | | | | |
| FAM210B | NA | 1162.51 | −11.02533 | uc002xxc.3 | chr20: 54941344- |
| | | | | | 54943718 |
| FAM213A | chr10: 82191856- | NA | NA | NA | NA |
| | 82192753 | | | | |
| FAM213B | NA | NA | NA | NA | NA |
| FAM215A | NA | NA | NA | NA | NA |
| FAM216A | chr12: 110927943- | NA | NA | NA | NA |
| | 110928192 | | | | |
| FAM216B | NA | NA | NA | NA | NA |
| FAM219B | NA | 230.672 | −5.087947 | uc002azh.4 | chr15: 75192328- |
| | | | | | 75194959 |
| FAM21C | NA | NA | NA | NA | NA |
| FAM221A | NA | NA | NA | NA | NA |
| FAM222B | NA | NA | NA | NA | NA |
| FAM227B | NA | 489.329 | −5.296089 | uc001zxl.2 | chr15: 49620592- |
| | | | | | 49620764 |
| FAM24A | chr10: 124672471- | NA | NA | NA | NA |
| | 124672627 | | | | |
| FAM24B | NA | NA | NA | NA | NA |
| FAM26E | NA | 1679.59 | −8.24003 | uc003pwy.3 | chr6: 116837153- |
| | | | | | 116839709 |
| FAM3D | NA | 460.335 | −4.96181 | uc003dkq.3 | chr3: 58619670- |
| | | | | | 58620005 |
| FAM46A | NA | NA | NA | NA | NA |
| FAM47A | chrX: 34147869- | NA | NA | NA | NA |
| | 34148019 | | | | |
| FAM47B | chrX: 34962887- | NA | NA | NA | NA |
| | 34963034 | | | | |
| FAM47C | NA | NA | NA | NA | NA |
| FAM49A | NA | 678.117 | −9.29512 | uc010exm.2 | chr2: 16730730- |
| | | | | | 16734203 |
| FAM50B | NA | 302.229 | −5.703427 | uc003mvu.3 | chr6: 3851024- |
| | | | | | 3851551 |
| FAM57A | NA | 299.267 | −7.350018 | uc002frp.3 | chr17: 644811- |
| | | | | | 646075 |
| FAM65C | NA | NA | NA | NA | NA |
| FAM69A | NA | 457.466 | −6.755109 | uc001dpc.3 | chr1: 93298286- |
| | | | | | 93298576 |
| FAM72B | chr1: 120854587- | 706.493 | −8.756299 | uc009whp.3 | chr1: 120854587- |
| | 120855681 | | | | 120855681 |
| FAM73A | chr1: 78324782- | NA | NA | NA | NA |
| | 78327860 | | | | |
| FAM73B | chr9: 131830813- | 1357.51 | −3.542979 | uc011mbn.1 | chr9: 131830813- |
| | 131831254 | | | | 131831254 |
| FAM76A | NA | 995.69 | −7.452681 | uc009vtb.3 | chr1: 28086169- |
| | | | | | 28086275 |
| FAM76B | chr11: 95502106- | NA | NA | NA | NA |
| | 95504724 | | | | |
| FAM81A | NA | 1030.32 | −11.057328 | uc010uha.2 | chr15: 59752583- |
| | | | | | 59752749 |
| FAM81B | NA | NA | NA | NA | NA |
| FAM83D | chr20: 37581164- | NA | NA | NA | NA |
| | 37581703 | | | | |
| FAM89A | chr1: 231154704- | NA | NA | NA | NA |
| | 231155608 | | | | |
| FAM91A1 | chr8: 124824945- | 970.639 | −5.968708 | uc011lik.1 | chr8: 124822631- |
| | 124827690 | | | | 124822719 |
| FAM92A1 | chr8: 94730903- | NA | NA | NA | NA |
| | 94731016 | | | | |
| FAM9A | chrX: 8758837- | NA | NA | NA | NA |
| | 8759213 | | | | |
| FAM9C | NA | 496.42 | −6.905571 | uc004cvg.3 | chrX: 13053736- |
| | | | | | 13056559 |
| FANCA | NA | 216.733 | −6.212881 | uc002fou.1 | chr16: 89803959- |
| | | | | | 89805008 |
| FANCD2 | chr3: 10081561- | NA | NA | NA | NA |
| | 10083246 | | | | |
| FANCE | NA | NA | NA | NA | NA |
| FANCF | NA | 679.091 | −6.674369 | uc001mql.1 | chr11: 22644079- |
| | | | | | 22646231 |
| FANCM | NA | 1117.07 | −20.64092 | uc001wwc.2 | chr14: 45636375- |
| | | | | | 45636626 |
| FANK1 | chr10: 127698008- | 870.421 | −5.981804 | uc001ljh.4 | chr10: 127698008- |
| | 127698161 | | | | 127698161 |
| FAP | chr2: 163027200- | NA | NA | NA | NA |
| | 163027488 | | | | |
| FARP1 | chr13: 99100572- | 447.946 | −5.128135 | uc001vnh.3 | chr13: 99100572- |
| | 99102023 | | | | 99102023 |
| FASTKD1 | NA | NA | NA | NA | NA |
| FASTKD2 | chr2: 207656527- | NA | NA | NA | NA |
| | 207660911 | | | | |
| FASTKD3 | NA | 287.522 | −4.876333 | uc011cmp.2 | chr5: 7859272- |
| | | | | | 7859547 |
| FAT2 | chr5: 150883653- | NA | NA | NA | NA |
| | 150885125 | | | | |
| FAU | chr11: 64888099- | NA | NA | NA | NA |
| | 64888152 | | | | |
| FAXDC2 | NA | 408.384 | −6.327885 | uc003lvs.4 | chr5: 154198052- |
| | | | | | 154199875 |
| FBLL1 | chr5: 167957515- | 1560.58 | −15.837406 | NA | NA |
| | 167957639 | | | | |
| FBLN2 | chr3: 13679420- | 292.2 | −4.628603 | uc011auz.2 | chr3: 13579420- |
| | 13679922 | | | | 13579922 |
| FBN2 | NA | 677.687 | −7.464141 | uc003kuv.2 | chr5: 127553781- |
| | | | | | 127664337 |
| FBP1 | NA | 212.126 | −5.087692 | uc004auw.4 | chr9: 97365421- |
| | | | | | 97365662 |
| FBP2 | chr9: 97321003- | NA | NA | NA | NA |
| | 97321219 | | | | |
| FBXL13 | NA | 607.961 | −5.432569 | uc010liq.1 | chr7: 102453308- |
| | | | | | 102453788 |
| FBXL17 | NA | NA | NA | NA | NA |
| FBXL22 | NA | 1432.94 | −6.021958 | uc002amm.2 | chr15: 63891393- |
| | | | | | 63892933 |
| FBXL3 | chr13: 77579389- | NA | NA | NA | NA |
| | 77581279 | | | | |
| FBXL4 | chr6: 99322468- | NA | NA | NA | NA |
| | 99323138 | | | | |
| FBXL5 | NA | NA | NA | NA | NA |
| FBXL6 | NA | NA | NA | NA | NA |
| FBXO11 | NA | 696.547 | −6.367636 | uc002rwg.2 | chr2: 48045555- |
| | | | | | 48045913 |
| FBXO15 | NA | 1602.49 | −8.788476 | uc002lle.2 | chr18: 71740588- |
| | | | | | 71740695 |
| FBXO21 | NA | NA | NA | NA | NA |
| FBXO22 | NA | 892.645 | −11.57123 | uc002bbj.2 | chr15: 76222428- |
| | | | | | 76223208 |
| FBXO25 | NA | NA | NA | NA | NA |
| FBXO30 | NA | 364.711 | −10.293061 | uc003qla.3 | chr6: 146119272- |
| | | | | | 146121241 |
| FBXO33 | NA | NA | NA | NA | NA |
| FBXO39 | NA | 515.227 | −5.428603 | uc010vtg.2 | chr17: 6690748- |
| | | | | | 6690965 |
| FBXO42 | NA | NA | NA | NA | NA |
| FBXO43 | chr8: 101145588- | 303.616 | −4.084102 | uc003yjd.3 | chr8: 101145588- |
| | 101146029 | | | | 101146029 |
| FBXO5 | chr6: 153291658- | NA | NA | NA | NA |
| | 153292297 | | | | |
| FBXO7 | NA | NA | NA | NA | NA |
| FBXW11 | NA | NA | NA | NA | NA |
| FBXW7 | chr4: 153332312- | NA | NA | NA | NA |
| | 153332424 | | | | |
| FCER1A | chr1: 159277723- | NA | NA | NA | NA |
| | 159278014 | | | | |
| FCER1G | chr1: 161188734- | NA | NA | NA | NA |
| | 161189038 | | | | |
| FCGR1A | NA | NA | NA | NA | NA |
| FCGR2A | NA | NA | NA | NA | NA |
| FCHO2 | chr5: 72333827- | NA | NA | NA | NA |
| | 72333938 | | | | |
| FCHSD2 | NA | 618.373 | −6.549 | uc001oth.4 | chr11: 72547790- |
| | | | | | 72549835 |
| FCRL3 | NA | NA | NA | NA | NA |
| FCRLB | NA | 606.777 | −7.838316 | uc001gbh.3 | chr1: 161697453- |
| | | | | | 161697933 |
| FDFT1 | NA | 1096.34 | −10.848559 | uc003wuh.3 | chr8: 11696119- |
| | | | | | 11696818 |
| FDX1 | chr11: 110333193- | NA | NA | NA | NA |
| | 110335608 | | | | |
| FDXACB1 | chr11: 111744780- | NA | NA | NA | NA |
| | 111745645 | | | | |
| FEN1 | chr11: 61563977- | NA | NA | NA | NA |
| | 61564714 | | | | |
| FER1L5 | NA | NA | NA | NA | NA |
| FERMT1 | chr20: 6055492- | NA | NA | NA | NA |
| | 6057819 | | | | |
| FEZ1 | NA | NA | NA | NA | NA |
| FGB | chr4: 155491803- | NA | NA | NA | NA |
| | 155493915 | | | | |
| FGD4 | chr12: 32793468- | 457.451 | −9.6022 | uc001rlc.3 | chr12: 32793468- |
| | 32798984 | | | | 32798984 |
| FGF1 | NA | NA | NA | NA | NA |
| FGF14 | chr13: 102373205- | NA | NA | NA | NA |
| | 102375180 | | | | |
| FGF20 | NA | NA | NA | NA | NA |
| FGF21 | NA | 1639.53 | −17.893559 | NA | NA |
| FGF7 | chr15: 49776702- | NA | NA | NA | NA |
| | 49779523 | | | | |
| FGFR1OP | NA | 460.806 | −9.720122 | uc011egp.1 | chr6: 167453608- |
| | | | | | 167453926 |
| FGL1 | chr8: 17721900- | 425.101 | −4.088876 | uc003wxx.3 | chr8: 17721900- |
| | 17722100 | | | | 17722100 |
| FGL2 | chr7: 76822688- | NA | NA | NA | NA |
| | 76825595 | | | | |
| FHL2 | chr2: 105977283- | 1317.91 | −7.489084 | uc002tde.1 | chr2: 105988004- |
| | 105977739 | | | | 105989986 |
| FHL5 | NA | 457.833 | −7.256791 | uc003pos.2 | chr6: 97063649- |
| | | | | | 97064512 |
| FIBIN | NA | 493.196 | −4.699684 | uc001mrd.3 | chr11: 27016710- |
| | | | | | 27018632 |
| FIGF | NA | NA | NA | NA | NA |
| FIGNL1 | chr7: 50511832- | NA | NA | NA | NA |
| | 50512960 | | | | |
| FILIP1 | NA | NA | NA | NA | NA |
| FILIP1L | chr3: 99566772- | 811.214 | −7.895483 | uc010hpf.3 | chr3: 99551988- |
| | 99567117 | | | | 99552078 |
| FIP1L1 | chr4: 54325617- | NA | NA | NA | NA |
| | 54326103 | | | | |
| FIS1 | NA | NA | NA | NA | NA |
| FITM2 | NA | NA | NA | NA | NA |
| FKBP15 | NA | NA | NA | NA | NA |
| FKBP6 | NA | 562.567 | −5.826351 | uc003twz.2 | chr7: 72772190- |
| | | | | | 72772641 |
| FKRP | NA | NA | NA | NA | NA |
| FKSG29 | chr13: 100004106- | NA | NA | NA | NA |
| | 100004281 | | | | |
| FLCN | NA | 1016.39 | −14.954518 | uc002gra.4 | chr17: 17115527- |
| | | | | | 17116968 |
| FLJ10038 | NA | 488.362 | −7.110787 | uc001zyg.3 | chr15: 50641135- |
| | | | | | 50646482 |
| FLJ13224 | chr12: 31478258- | NA | NA | NA | NA |
| | 31478879 | | | | |
| FLJ30679 | NA | NA | NA | NA | NA |
| FLJ33360 | NA | NA | NA | NA | NA |
| FLJ44635 | chrX: 71380103- | NA | NA | NA | NA |
| | 71381600 | | | | |
| FLNB | chr3: 58156490- | 751.361 | −9.488768 | uc003djj.2 | chr3: 58156490- |
| | 58157982 | | | | 58157982 |
| FLT4 | NA | 908.216 | −6.552262 | uc003mma.4 | chr5: 180034753- |
| | | | | | 180035280 |
| FLVCR1 | NA | NA | NA | NA | NA |
| FMN2 | NA | 1129.02 | −8.865061 | uc010pyd.2 | chr1: 240637455- |
| | | | | | 240638489 |
| FMOD | NA | 494.091 | −6.740556 | uc001gzr.3 | chr1: 203309752- |
| | | | | | 203311470 |
| FN1 | NA | 939.179 | −9.601432 | uc002vez.3 | chr2: 216225179- |
| | | | | | 216226277 |
| FN3KRP | NA | NA | NA | NA | NA |
| FNBP4 | NA | 951.412 | −11.825676 | uc001ngj.3 | chr11: 47738069- |
| | | | | | 47738973 |
| FNDC3A | chr13: 49720076- | NA | NA | NA | NA |
| | 49720983 | | | | |
| FNDC3B | NA | NA | NA | NA | NA |
| FNTA | NA | NA | NA | NA | NA |
| FOLR1 | chr11: 71907222- | NA | NA | NA | NA |
| | 71907367 | | | | |
| FOLR2 | chr11: 71932807- | NA | NA | NA | NA |
| | 71932994 | | | | |
| FOPNL | chr16: 15959577- | NA | NA | NA | NA |
| | 15961296 | | | | |
| FOXA3 | NA | NA | NA | NA | NA |
| FOXK2 | chr17: 80553717- | 441.531 | −7.543716 | uc010diu.3 | chr17: 80559281- |
| | 80554067 | | | | 80562483 |
| FOXN3 | chr14: 89622516- | NA | NA | NA | NA |
| | 89628757 | | | | |
| FOXP1 | chr3: 71003865- | NA | NA | NA | NA |
| | 71008397 | | | | |
| FOXP2 | chr7: 114284849- | NA | NA | NA | NA |
| | 114285885 | | | | |
| FPGS | NA | 846.978 | −7.136163 | uc004bsg.1 | chr9: 130575884- |
| | | | | | 130576556 |
| FPGT | chr1: 74671517- | NA | NA | NA | NA |
| | 74674148 | | | | |
| FRA10AC1 | chr10: 95448837- | 1224.8 | −7.525986 | uc009xuh.1 | chr10: 95451529- |
| | 95451720 | | | | 95452398 |
| FREM1 | NA | 596.884 | −7.088693 | uc003zll.3 | chr9: 14734664- |
| | | | | | 14737393 |
| FREM2 | chr13: 39438765- | NA | NA | NA | NA |
| | 39439063 | | | | |
| FRG1 | NA | 1090.99 | −8.532667 | uc003izs.3 | chr4: 190884285- |
| | | | | | 190884359 |
| FRG1B | chr20: 29652155- | NA | NA | NA | NA |
| | 29653908 | | | | |
| FRG2EP | NA | 235.695 | −4.814194 | uc003dpt.4 | chr3: 75715193- |
| | | | | | 75716368 |
| FRMD4B | chr3: 69219146- | NA | NA | NA | NA |
| | 69221011 | | | | |
| FRMD6 | NA | NA | NA | NA | NA |
| FRMD7 | chrX: 131211021- | NA | NA | NA | NA |
| | 131211899 | | | | |
| FRMD8 | NA | 233.211 | −5.387098 | uc001odu.4 | chr11: 65178832- |
| | | | | | 65180995 |
| FRMPD2 | NA | 716.916 | −7.073337 | uc001jgg.3 | chr10: 49364601- |
| | | | | | 49365364 |
| FRMPD2B | NA | 464.764 | −6.266805 | uc001jfy.4 | chr10: 48844036- |
| | | | | | 48844799 |
| FRRS1L | NA | 701.85 | −7.929096 | uc004bdw.1 | chr9: 111899581- |
| | | | | | 111899734 |
| FRS2 | chr12: 69968736- | NA | NA | NA | NA |
| | 69973562 | | | | |
| FRYL | NA | 274.035 | −4.164841 | uc003gym.1 | chr4: 48621544- |
| | | | | | 48622651 |
| FRZB | chr2: 183698005- | NA | NA | NA | NA |
| | 183699575 | | | | |
| FSIP1 | chr15: 39892232- | 220.54 | −4.179799 | uc001zki.3 | chr15: 39892232- |
| | 39893080 | | | | 39893080 |
| FSTL3 | NA | NA | NA | NA | NA |
| FSTL4 | chr5: 132532152- | NA | NA | NA | NA |
| | 132534786 | | | | |
| FTHL17 | NA | NA | NA | NA | NA |
| FTMT | NA | 611.867 | −6.379406 | uc003kss.3 | chr5: 121188388- |
| | | | | | 121188523 |
| FTSJ2 | NA | 660.441 | −9.866254 | uc003slm.3 | chr7: 2273926- |
| | | | | | 2274756 |
| FUBP1 | NA | 1229.93 | −9.787264 | uc001dii.3 | chr1: 78413591- |
| | | | | | 78414450 |
| FUBP3 | NA | NA | NA | NA | NA |
| FUCA1 | chr1: 24171572- | NA | NA | NA | NA |
| | 24172204 | | | | |
| FUCA2 | NA | NA | NA | NA | NA |
| FUNDC2 | NA | 523.766 | −5.296089 | uc004fmw.3 | chrX: 154282948- |
| | | | | | 154285191 |
| FUNDC2P2 | chr2: 84518399- | NA | NA | NA | NA |
| | 84519324 | | | | |
| FUOM | NA | NA | NA | NA | NA |
| FUS | NA | 688.693 | −4.840821 | uc010vfi.2 | chr16: 31194165- |
| | | | | | 31194671 |
| FUT10 | NA | 835.767 | −4.498625 | uc003xjc.3 | chr8: 33228344- |
| | | | | | 33230094 |
| FUT11 | chr10: 75533108- | 304.448 | −6.493437 | uc001jva.3 | chr10: 75535444- |
| | 75534881 | | | | 75535976 |
| FUT4 | chr11: 94278893- | NA | NA | NA | NA |
| | 94283064 | | | | |
| FUT9 | NA | NA | NA | NA | NA |
| FUZ | chr19: 50310124- | NA | NA | NA | NA |
| | 50310407 | | | | |
| FXN | NA | 315.878 | −5.652384 | uc004agz.2 | chr9: 71687629- |
| | | | | | 71693993 |
| FXYD5 | NA | 493.555 | −7.094459 | uc010xsq.2 | chr19: 35657327- |
| | | | | | 35657664 |
| FYB | NA | 1138.85 | −9.754634 | uc003jls.3 | chr5: 39105354- |
| | | | | | 39107544 |
| FZD10 | NA | 1650.6 | −10.873198 | uc001uii.3 | chr12: 130649234- |
| | | | | | 130650285 |
| FZD2 | chr17: 42636755- | NA | NA | NA | NA |
| | 42638630 | | | | |
| FZD4 | NA | 307.351 | −6.776111 | uc001pce.3 | chr11: 86656717- |
| | | | | | 86662183 |
| FZD6 | NA | NA | NA | NA | NA |
| G2E3 | NA | NA | NA | NA | NA |
| G3BP2 | chr4: 76567953- | 1940.46 | −14.661271 | uc003hir.3 | chr4: 76567953- |
| | 76570613 | | | | 76570613 |
| GAB3 | chrX: 153903527- | NA | NA | NA | NA |
| | 153906454 | | | | |
| GAB4 | chr22: 17442827- | NA | NA | NA | NA |
| | 17443622 | | | | |
| GABPB1 | chr15: 50578077- | NA | NA | NA | NA |
| | 50578216 | | | | |
| GABRA1 | chr5: 161324429- | 440.105 | −6.984171 | uc010jiw.3 | chr5: 161324429- |
| | 161326965 | | | | 161326965 |
| GABRA6 | chr5: 161128780- | NA | NA | NA | NA |
| | 161129598 | | | | |
| GABRG2 | chr5: 161580375- | NA | NA | NA | NA |
| | 161582545 | | | | |
| GABRR1 | NA | NA | NA | NA | NA |
| GADD45A | NA | NA | NA | NA | NA |
| GALC | chr14: 88399358- | NA | NA | NA | NA |
| | 88401075 | | | | |
| GALNS | chr16: 88921536- | 834.369 | −6.906133 | uc002fma.3 | chr16: 88921536- |
| | 88921794 | | | | 88921794 |
| GALNT13 | chr2: 155307064- | 297.384 | −3.800905 | uc010foc.1 | chr2: 155265694- |
| | 155310489 | | | | 155265852 |
| GALNT15 | NA | NA | NA | NA | NA |
| GALNT3 | NA | NA | NA | NA | NA |
| GALNT8 | NA | 635.617 | −8.723398 | uc001qne.1 | chr12: 4881764- |
| | | | | | 4881892 |
| GALP | chr19: 56696640- | 460.58 | −1.35247 | uc002qmo.1 | chr19: 56696640- |
| | 56697144 | | | | 56697144 |
| GALR1 | NA | 370.443 | −5.866022 | uc002lms.4 | chr18: 74980859- |
| | | | | | 74982096 |
| GAPT | NA | 398.497 | −7.736926 | uc003jro.1 | chr5: 57790838- |
| | | | | | 57792185 |
| GAR1 | NA | 655.206 | −5.301186 | uc003hzt.3 | chr4: 110745588- |
| | | | | | 110745893 |
| GARNL3 | chr9: 130155534- | 351.537 | −5.234056 | uc004bqt.1 | chr9: 130106658- |
| | 130155828 | | | | 130107617 |
| GART | NA | NA | NA | NA | NA |
| GAS2 | NA | 851.976 | −11.062559 | uc009yie.3 | chr11: 22833563- |
| | | | | | 22834547 |
| GAS2L1 | NA | NA | NA | NA | NA |
| GAS8 | chr16: 90109754- | NA | NA | NA | NA |
| | 90111379 | | | | |
| GATA1 | chrX: 48652572- | NA | NA | NA | NA |
| | 48652717 | | | | |
| GATS | chr7: 99798278- | NA | NA | NA | NA |
| | 99800226 | | | | |
| GBAS | chr7: 56066766- | NA | NA | NA | NA |
| | 56067875 | | | | |
| GBE1 | NA | NA | NA | NA | NA |
| GBP3 | chr1: 89472360- | NA | NA | NA | NA |
| | 89473406 | | | | |
| GBP5 | chr1: 89724634- | NA | NA | NA | NA |
| | 89726386 | | | | |
| GBP6 | NA | NA | NA | NA | NA |
| GBP7 | NA | 414.586 | −10.684653 | uc001dna.2 | chr1: 89597434- |
| | | | | | 89597831 |
| GCA | NA | NA | NA | NA | NA |
| GCC2 | NA | NA | NA | NA | NA |
| GCH1 | NA | NA | NA | NA | NA |
| GCLC | NA | 1356.12 | −9.692521 | uc003pbv.1 | chr6: 53362140- |
| | | | | | 53363553 |
| GCN1L1 | NA | 718.516 | −7.577541 | uc001txo.3 | chr12: 120565014- |
| | | | | | 120565652 |
| GCNT7 | chr20: 55066548- | 873.676 | −8.057442 | uc010zzg.1 | chr20: 55066548- |
| | 55066612 | | | | 55066612 |
| GDA | chr9: 74863259- | 1009.33 | −4.307779 | uc011lse.2 | chr9: 74863259- |
| | 74867140 | | | | 74867140 |
| GDAP1 | chr8: 75276603- | NA | NA | NA | NA |
| | 75279335 | | | | |
| GDF15 | NA | 1055.78 | −11.622865 | uc002niv.2 | chr19: 18499746- |
| | | | | | 18499986 |
| GDNF | NA | NA | NA | NA | NA |
| GDPD1 | NA | 336.464 | −5.678282 | uc002ixk.2 | chr17: 57351135- |
| | | | | | 57353330 |
| GDPD5 | NA | NA | NA | NA | NA |
| GEMIN7 | chr19: 45593769- | NA | NA | NA | NA |
| | 45594782 | | | | |
| GEMIN8 | NA | 1131.02 | −4.980835 | uc004cwb.3 | chrX: 14024845- |
| | | | | | 14027031 |
| GEN1 | NA | 854.405 | −7.545694 | uc002rct.2 | chr2: 17963207- |
| | | | | | 17966632 |
| GFM1 | chr3: 158399959- | NA | NA | NA | NA |
| | 158400222 | | | | |
| GGA2 | NA | NA | NA | NA | NA |
| GGT5 | NA | NA | NA | NA | NA |
| GHRL | NA | 868.58 | −4.605842 | uc003bvj.1 | chr3: 10327434- |
| | | | | | 10327527 |
| GID4 | chr17: 17968558- | NA | NA | NA | NA |
| | 17971718 | | | | |
| GID8 | NA | 316.656 | −4.031126 | uc002ydy.3 | chr20: 61576265- |
| | | | | | 61579827 |
| GIF | NA | NA | NA | NA | NA |
| GIMAP2 | NA | 412.609 | −6.407206 | uc003who.3 | chr7: 150390389- |
| | | | | | 150390728 |
| GIMAP4 | chr7: 150270149- | 1090.19 | −8.427373 | uc011kuu.2 | chr7: 150270149- |
| | 150270308 | | | | 150270308 |
| GINS3 | NA | NA | NA | NA | NA |
| GIPC2 | chr1: 78601428- | NA | NA | NA | NA |
| | 78603112 | | | | |
| GIT2 | chr12: 110367607- | 347.697 | −6.887039 | uc001tpq.2 | chr12: 110367607- |
| | 110370782 | | | | 110370782 |
| GJA3 | NA | 594.313 | −8.586587 | uc001umx.1 | chr13: 20712395- |
| | | | | | 20716119 |
| GJB3 | chr1: 35251177- | NA | NA | NA | NA |
| | 35251967 | | | | |
| GJC1 | NA | 546.21 | −8.060838 | uc002ihj.3 | chr17: 42875816- |
| | | | | | 42881994 |
| GJC3 | NA | NA | NA | NA | NA |
| GKAP1 | NA | 385.637 | −5.461205 | uc004amy.3 | chr9: 86354336- |
| | | | | | 86354611 |
| GLA | chrX: 100656118- | NA | NA | NA | NA |
| | 100656530 | | | | |
| GLDN | NA | NA | NA | NA | NA |
| GLE1 | NA | 838.893 | −7.369056 | uc004bvj.3 | chr9: 131303450- |
| | | | | | 131304580 |
| GLG1 | NA | 1282.86 | −10.380517 | uc002fcw.4 | chr16: 74481326- |
| | | | | | 74485953 |
| GLI2 | NA | 374.052 | −4.609851 | uc010yyu.1 | chr2: 121709030- |
| | | | | | 121709339 |
| GLI3 | NA | NA | NA | NA | NA |
| GLIPR2 | NA | 740.209 | −8.203101 | uc011lpj.1 | chr9: 36151033- |
| | | | | | 36151768 |
| GLIS3 | chr9: 3855437- | 552.569 | −7.744022 | uc010mhf.1 | chr9: 3824128- |
| | 3855967 | | | | 3828271 |
| GLRA2 | chrX: 14748608- | NA | NA | NA | NA |
| | 14749933 | | | | |
| GLRA3 | NA | 947.907 | −12.449671 | uc003ity.1 | chr4: 175563198- |
| | | | | | 175564936 |
| GLRA4 | NA | 859.062 | −8.585353 | uc010nou.2 | chrX: 102973502- |
| | | | | | 102973888 |
| GLRB | NA | 449.626 | −6.470214 | uc003ipj.2 | chr4: 158091881- |
| | | | | | 158093242 |
| GLRX | NA | 519.337 | −4.565581 | uc003kln.4 | chr5 95149553- |
| | | | | | 95150661 |
| GLRX2 | NA | 679.554 | −10.139386 | uc001gsz.2 | chr1: 193065595- |
| | | | | | 193065754 |
| GLRX3 | NA | 208.41 | −4.641606 | uc001lkn.2 | chr10: 131978377- |
| | | | | | 131978646 |
| GLRX5 | chr14: 96010463- | NA | NA | NA | NA |
| | 96011055 | | | | |
| GLS | chr2: 191760002- | NA | NA | NA | NA |
| | 191761227 | | | | |
| GM2A | NA | NA | NA | NA | NA |
| GMEB1 | NA | 1483.91 | −17.804893 | uc001bqz.3 | chr1: 29041286- |
| | | | | | 29042115 |
| GMPPB | NA | NA | NA | NA | NA |
| GNB2L1 | chr5: 180666873- | NA | NA | NA | NA |
| | 180667312 | | | | |
| GNB3 | chr12: 6956063- | NA | NA | NA | NA |
| | 6956557 | | | | |
| GNB4 | chr3: 179113876- | NA | NA | NA | NA |
| | 179119000 | | | | |
| GNG7 | chr19: 2511218- | 819.106 | −10.023752 | uc002lwd.2 | chr19: 2511218- |
| | 2515019 | | | | 2515019 |
| GNL1 | chr6: 30509155- | 666.722 | −3.901655 | uc011dmi.2 | chr6: 30509155- |
| | 30513848 | | | | 30513848 |
| GNL3 | chr3: 52728292- | 823.569 | −8.203496 | uc003dfd.3 | chr3: 52728292- |
| | 52728510 | | | | 52728510 |
| GNN | chr12: 104307805- | NA | NA | NA | NA |
| | 104309225 | | | | |
| GNPTAB | NA | 408.546 | −7.350016 | uc001tit.3 | chr12: 102139275- |
| | | | | | 102140941 |
| GNRHR | chr4: 68603099- | NA | NA | NA | NA |
| | 68606306 | | | | |
| GNS | chr12: 65107222- | NA | NA | NA | NA |
| | 65110520 | | | | |
| GOLGA3 | chr12: 133345495- | NA | NA | NA | NA |
| | 133349690 | | | | |
| GOLGA6B | NA | NA | NA | NA | NA |
| GOLGA6L10 | NA | 536.439 | −0.526154 | uc021ssn.1 | chr15: 82633123- |
| | | | | | 82635129 |
| GOLGA8IP | NA | NA | NA | NA | NA |
| GOLPH3 | chr5: 32124824- | NA | NA | NA | NA |
| | 32126317 | | | | |
| GOLT1B | NA | NA | NA | NA | NA |
| GON4L | NA | 1465.21 | −16.83926 | uc001fmc.3 | chr1: 155734576- |
| | | | | | 155734673 |
| GORASP2 | chr2: 171822641- | NA | NA | NA | NA |
| | 171823643 | | | | |
| GOSR1 | NA | 1061.94 | −10.747866 | uc002hfc.1 | chr17: 28811882- |
| | | | | | 28812280 |
| GOSR2 | chr17: 45012647- | NA | NA | NA | NA |
| | 45014205 | | | | |
| GP6 | chr19: 55525075- | NA | NA | NA | NA |
| | 55525449 | | | | |
| GPALPP1 | NA | 707.396 | −4.582683 | uc001uzq.3 | chr13: 45602139- |
| | | | | | 45602404 |
| GPAM | chr10: 113914758- | NA | NA | NA | NA |
| | 113916994 | | | | |
| GPC3 | chrX: 132669776- | 1145.21 | −10.450789 | uc004exe.2 | chrX: 132669776- |
| | 132670151 | | | | 132670151 |
| GPC5 | NA | NA | NA | NA | NA |
| GPC6 | chr13: 94958513- | 960.96 | −8.751567 | uc001vlu.1 | chr13: 94482868- |
| | 94958788 | | | | 94484342 |
| GPCPD1 | chr20: 5525080- | 671.138 | −8.302363 | uc002wmd.4 | chr20: 5525080- |
| | 5528306 | | | | 5528306 |
| GPHA2 | NA | 633.004 | −5.68575 | uc001oca.3 | chr11: 64701943- |
| | | | | | 64702244 |
| GPHN | chr14: 67525557- | NA | NA | NA | NA |
| | 67525733 | | | | |
| GPKOW | NA | 539.771 | −5.144435 | uc004dmr.3 | chrX: 48969022- |
| | | | | | 48970558 |
| GPLD1 | chr6: 24447759- | NA | NA | NA | NA |
| | 24448093 | | | | |
| GPM6A | chr4: 176554088- | NA | NA | NA | NA |
| | 176556055 | | | | |
| GPN3 | chr12: 110890291- | NA | NA | NA | NA |
| | 110890838 | | | | |
| GPR1 | NA | 395.275 | −3.439412 | uc002vbl.4 | chr2: 207040042- |
| | | | | | 207040903 |
| GPR137C | NA | NA | NA | NA | NA |
| GPR157 | NA | 508.056 | −5.101542 | uc001apr.3 | chr1: 9169158- |
| | | | | | 9171214 |
| GPR171 | chr3: 150915619- | NA | NA | NA | NA |
| | 150916213 | | | | |
| GPR19 | NA | NA | NA | NA | NA |
| GPR35 | NA | 465.086 | −4.819481 | uc010fzh.2 | chr2: 241570300- |
| | | | | | 241570676 |
| GPR37L1 | NA | NA | NA | NA | NA |
| GPR4 | NA | NA | NA | NA | NA |
| GPR45 | chr2: 105859435- | NA | NA | NA | NA |
| | 105859924 | | | | |
| GPR63 | chr6: 97241998- | NA | NA | NA | NA |
| | 97246347 | | | | |
| GPR75 | NA | NA | NA | NA | NA |
| GPR82 | NA | NA | NA | NA | NA |
| GPR84 | NA | NA | NA | NA | NA |
| GPR87 | NA | 573.915 | −5.801158 | uc003eyt.2 | chr3: 151011876- |
| | | | | | 151011956 |
| GPRASP1 | NA | 585.914 | −6.558034 | uc004eji.4 | chrX: 101913030- |
| | | | | | 101914010 |
| GPRIN3 | chr4: 90165429- | NA | NA | NA | NA |
| | 90168930 | | | | |
| GPSM2 | chr1: 109472563- | 719.784 | −0.823285 | uc010ovc.2 | chr1: 109472563- |
| | 109473044 | | | | 109473044 |
| GPX1 | NA | 615.166 | −7.258569 | uc021wxw.1 | chr3: 49394609- |
| | | | | | 49394820 |
| GPX8 | NA | 281.34 | −6.998558 | uc003jpr.2 | chr5: 54459952- |
| | | | | | 54463129 |
| GRAMD2 | chr15: 72452147- | NA | NA | NA | NA |
| | 72454349 | | | | |
| GRAMD3 | NA | 229.647 | −4.482767 | uc011cwt.2 | chr5: 125828649- |
| | | | | | 125829853 |
| GRAMD4 | chr22: 47073149- | 1200.48 | −10.957936 | uc003bhx.3 | chr22: 47073149- |
| | 47075688 | | | | 47075688 |
| GRAP2 | NA | 715.278 | −7.248455 | uc003ayh.2 | chr22: 40367089- |
| | | | | | 40367384 |
| GREB1L | NA | 420.649 | −2.431635 | uc002ktf.1 | chr18: 19034599- |
| | | | | | 19035091 |
| GRHL2 | NA | 440.862 | −5.490174 | uc010mbt.1 | chr8: 102555788- |
| | | | | | 102556298 |
| GRIA2 | NA | NA | NA | NA | NA |
| GRIA3 | NA | NA | NA | NA | NA |
| GRIA4 | NA | NA | NA | NA | NA |
| GRID1 | NA | 563.307 | −6.678257 | uc010qmf.1 | chr10: 87359312- |
| | | | | | 87362029 |
| GRIK1 | NA | NA | NA | NA | NA |
| GRIK2 | chr6: 102337743- | 777.659 | −1.386778 | uc021zdj.1 | chr6: 102192585- |
| | 102338007 | | | | 102193018 |
| GRIN3A | chr9: 104337982- | NA | NA | NA | NA |
| | 104340045 | | | | |
| GRM7 | chr3: 7782094- | NA | NA | NA | NA |
| | 7783218 | | | | |
| GRM8 | NA | NA | NA | NA | NA |
| GRP | chr18: 56897690- | NA | NA | NA | NA |
| | 56898002 | | | | |
| GRPEL2 | chr5: 148730846- | 583.489 | −5.443081 | uc003lqj.3 | chr5: 148730846- |
| | 148734146 | | | | 148734146 |
| GRSF1 | chr4: 71681499- | NA | NA | NA | NA |
| | 71686578 | | | | |
| GRTP1 | NA | NA | NA | NA | NA |
| GRXCR1 | NA | 664.001 | −4.486431 | uc003gwt.3 | chr4: 43032558- |
| | | | | | 43032675 |
| GSDMB | chr17: 38060848- | NA | NA | NA | NA |
| | 38061064 | | | | |
| GSDMC | chr8: 130760442- | NA | NA | NA | NA |
| | 130760746 | | | | |
| GSR | chr8: 30535580- | NA | NA | NA | NA |
| | 30537036 | | | | |
| GSTA1 | NA | 523.709 | −6.476277 | uc003paz.3 | chr6: 52656178- |
| | | | | | 52656655 |
| GSTM2 | NA | NA | NA | NA | NA |
| GSTM3 | chr1: 110276554- | NA | NA | NA | NA |
| | 110279692 | | | | |
| GSTM4 | NA | NA | NA | NA | NA |
| GSTM5 | NA | 773.153 | −4.352682 | uc001dyn.3 | chr1: 110260049- |
| | | | | | 110260890 |
| GSTO2 | chr10: 106059043- | 484.01 | −13.616393 | uc010qqw.1 | chr10: 106048393- |
| | 106059176 | | | | 106048815 |
| GTDC1 | chr2: 144703581- | NA | NA | NA | NA |
| | 144704702 | | | | |
| GTF2A2 | NA | 1132.57 | −7.260382 | uc002agg.3 | chr15: 59930261- |
| | | | | | 59931330 |
| GTF2E2 | NA | NA | NA | NA | NA |
| GTF2H2C | NA | 281.659 | −7.915613 | uc003jww.1 | chr5: 68874584- |
| | | | | | 68874629 |
| GTF2I | chr7: 74131276- | 1221.5 | −5.455973 | uc003uba.2 | chr7: 74173169- |
| | 74131386 | | | | 74175020 |
| GTF2IRD2B | chr7: 74539125- | NA | NA | NA | NA |
| | 74540018 | | | | |
| GTF3C3 | chr2: 197627756- | NA | NA | NA | NA |
| | 197629286 | | | | |
| GTF3C6 | NA | NA | NA | NA | NA |
| GTPBP2 | NA | NA | NA | NA | NA |
| GTPBP4 | NA | NA | NA | NA | NA |
| GTPBP6 | NA | NA | NA | NA | NA |
| GTSF1 | chr12: 54849736- | NA | NA | NA | NA |
| | 54849937 | | | | |
| GUCA1A | NA | 609.245 | −6.359267 | uc003orx.3 | chr6: 42147142- |
| | | | | | 42147794 |
| GUCY1A3 | NA | NA | NA | NA | NA |
| GUF1 | chr4: 44700699- | NA | NA | NA | NA |
| | 44702697 | | | | |
| GULP1 | NA | NA | NA | NA | NA |
| GUSB | NA | 604.112 | −7.304119 | uc003tun.3 | chr7: 65425673- |
| | | | | | 65425883 |
| GUSBP1 | NA | 750.716 | −5.3711 | uc003jgf.3 | chr5: 21470807- |
| | | | | | 21471090 |
| GYG1 | NA | 948.431 | −3.215325 | uc003ewn.3 | chr3: 148744721- |
| | | | | | 148745456 |
| GYG2 | NA | NA | NA | NA | NA |
| GYG2P1 | NA | 1151.16 | −7.772186 | uc022cji.1 | chrY: 14517915- |
| | | | | | 14518274 |
| GYPE | NA | 1028.84 | −5.799805 | uc003ijj.3 | chr4: 144792019- |
| | | | | | 144793405 |
| GZF1 | NA | NA | NA | NA | NA |
| GZMK | chr5: 54329755- | NA | NA | NA | NA |
| | 54329960 | | | | |
| H1F0 | NA | 1323.99 | −10.044178 | uc003aty.3 | chr22: 38202137- |
| | | | | | 38203443 |
| H1FX | NA | 654.284 | −6.85454 | uc003elx.3 | chr3: 129033614- |
| | | | | | 129034103 |
| H2AFY | chr5: 134670071- | NA | NA | NA | NA |
| | 134670665 | | | | |
| H2AFZ | chr4: 100869243- | 1115.95 | −4.529025 | uc003hvn.1 | chr4: 100869243- |
| | 100870603 | | | | 100870603 |
| H3F3C | NA | NA | NA | NA | NA |
| HACD3 | NA | NA | NA | NA | NA |
| HACD4 | chr9: 21003623- | NA | NA | NA | NA |
| | 21007035 | | | | |
| HADHA | chr2: 26434806- | NA | NA | NA | NA |
| | 26435369 | | | | |
| HAGH | NA | NA | NA | NA | NA |
| HAO1 | chr20: 7863631- | 907.79 | −2.680614 | uc002wmw.1 | chr20: 7863631- |
| | 7864239 | | | | 7864239 |
| HAP1 | NA | NA | NA | NA | NA |
| HARS | chr5: 140053490- | NA | NA | NA | NA |
| | 140053841 | | | | |
| HARS2 | NA | NA | NA | NA | NA |
| HAS2 | NA | 452.084 | −8.858592 | uc003yph.2 | chr8: 122625271- |
| | | | | | 122626348 |
| HAS3 | NA | 878.021 | −6.750959 | uc010cfh.3 | chr16: 69149170- |
| | | | | | 69151570 |
| HAT1 | NA | NA | NA | NA | NA |
| HAUS2 | chr15: 42859015- | NA | NA | NA | NA |
| | 42862190 | | | | |
| HAUS3 | NA | NA | NA | NA | NA |
| HAUS4 | chr14: 23415437- | NA | NA | NA | NA |
| | 23415733 | | | | |
| HAUS8 | NA | NA | NA | NA | NA |
| HAVCR1 | chr5: 156456531- | NA | NA | NA | NA |
| | 156456598 | | | | |
| HAVCR2 | chr5: 156512843- | 1018.94 | −7.264807 | uc003lwk.2 | chr5: 156512843- |
| | 156514112 | | | | 156514112 |
| HBB | NA | NA | NA | NA | NA |
| HBG1 | chr11: 5269502- | NA | NA | NA | NA |
| | 5269588 | | | | |
| HBG2 | chr11: 5269502- | NA | NA | NA | NA |
| | 5269588 | | | | |
| HBP1 | NA | 844.066 | −5.659931 | uc011klv.2 | chr7: 106841877- |
| | | | | | 106842974 |
| HCCS | NA | 493.94 | −4.919721 | uc004cuj.3 | chrX: 11139931- |
| | | | | | 11141204 |
| HCFC2 | NA | NA | NA | NA | NA |
| HCN1 | NA | 896.746 | −8.295173 | uc003jok.3 | chr5: 45255052- |
| | | | | | 45262022 |
| HCRTR1 | NA | 238.217 | −4.925141 | uc009vtx.2 | chr1: 32092582- |
| | | | | | 32092919 |
| HDAC1 | NA | NA | NA | NA | NA |
| HDAC11 | chr3: 13546184- | NA | NA | NA | NA |
| | 13547924 | | | | |
| HDAC5 | NA | NA | NA | NA | NA |
| HDDC2 | chr6: 125596496- | 289.983 | −10.674499 | uc003qaa.1 | chr6: 125596496- |
| | 125597291 | | | | 125597291 |
| HDHD2 | NA | 416.882 | −6.391384 | uc002lcs.3 | chr18: 44633781- |
| | | | | | 44635052 |
| HDLBP | NA | 678.416 | −4.868837 | uc002waz.3 | chr2: 242166682- |
| | | | | | 242169015 |
| HDX | chrX: 83572882- | NA | NA | NA | NA |
| | 83576896 | | | | |
| HEATR1 | NA | 1354.11 | −17.402569 | uc009xgh.2 | chr1: 236712305- |
| | | | | | 236714201 |
| HEATR5A | chr14: 31803518- | NA | NA | NA | NA |
| | 31803606 | | | | |
| HEATR5B | NA | 603.061 | −5.432614 | uc010ezy.1 | chr2: 37208153- |
| | | | | | 37208632 |
| HEATR9 | chr17: 34181960- | NA | NA | NA | NA |
| | 34182066 | | | | |
| HECA | chr6: 139498243- | NA | NA | NA | NA |
| | 139501946 | | | | |
| HECTD1 | NA | 408.483 | −5.724804 | uc001wra.1 | chr14: 31569324- |
| | | | | | 31570135 |
| HECTD2 | chr10: 93221966- | 986.993 | −7.728285 | uc001khk.2 | chr10: 93221966- |
| | 93222967 | | | | 93222967 |
| HECTD3 | NA | 472.745 | −4.570054 | uc001cmx.4 | chr1: 45468220- |
| | | | | | 45469163 |
| HECTD4 | NA | NA | NA | NA | NA |
| HELLS | NA | 684.372 | −9.271534 | uc001kjs.3 | chr10: 96361380- |
| | | | | | 96361856 |
| HELQ | NA | NA | NA | NA | NA |
| HELZ | chr17: 65066554- | 601.445 | −6.541207 | uc010der.3 | chr17: 65104267- |
| | 65074367 | | | | 65104546 |
| HEPACAM2 | NA | 311.766 | −5.603029 | uc010lff.3 | chr7: 92817899- |
| | | | | | 92818539 |
| HEPH | chrX: 65486515- | NA | NA | NA | NA |
| | 65487230 | | | | |
| HERC1 | NA | NA | NA | NA | NA |
| HERC2 | NA | NA | NA | NA | NA |
| HERC4 | chr10: 69681656- | NA | NA | NA | NA |
| | 69682687 | | | | |
| HEXB | NA | NA | NA | NA | NA |
| HEXDC | chr17: 80398117- | NA | NA | NA | NA |
| | 80398408 | | | | |
| HEXIM1 | chr17: 43227638- | NA | NA | NA | NA |
| | 43229468 | | | | |
| HHIP | chr4: 145659110- | NA | NA | NA | NA |
| | 145659881 | | | | |
| HIATL1 | NA | 292.197 | −5.767179 | uc004aur.3 | chr9: 97221695- |
| | | | | | 97223202 |
| HIGD1A | chr3: 42824400- | NA | NA | NA | NA |
| | 42826762 | | | | |
| HILPDA | NA | 574.707 | −6.05046 | uc003vne.4 | chr7: 128097515- |
| | | | | | 128098472 |
| HIP1R | NA | 402.888 | −4.676168 | uc001udg.1 | chr12: 123341796- |
| | | | | | 123342447 |
| HIPK1 | NA | 234.361 | −5.10492 | uc001eem.3 | chr1: 114516135- |
| | | | | | 114520491 |
| HIST1H2AB | chr6: 26033320- | NA | NA | NA | NA |
| | 26033403 | | | | |
| HIVEP1 | chr6: 12164695- | 1145.13 | −10.720855 | uc003nac.3 | chr6: 12164695- |
| | 12165232 | | | | 12165232 |
| HIVEP2 | NA | 590.38 | −7.042919 | uc003qjd.3 | chr6: 143072604- |
| | | | | | 143074243 |
| HJURP | NA | 634.485 | −7.534397 | uc002wg.3 | chr2: 234745486- |
| | | | | | 234746222 |
| HKR1 | NA | 264.521 | −8.162035 | uc002ofz.3 | chr19: 37850588- |
| | | | | | 37850676 |
| HLF | NA | NA | NA | NA | NA |
| HLTF | chr3: 148747904- | NA | NA | NA | NA |
| | 148749150 | | | | |
| HMBOX1 | NA | 713.649 | −8.716263 | uc010lvd.3 | chr8: 28906632- |
| | | | | | 28906838 |
| HMBS | NA | 922.199 | −7.905713 | uc009zao.2 | chr11: 118962904- |
| | | | | | 118963521 |
| HMCES | chr3: 129023669- | NA | NA | NA | NA |
| | 129024135 | | | | |
| HMG20A | NA | 503.862 | −5.723396 | uc002bcq.1 | chr15: 77756739- |
| | | | | | 77757355 |
| HMGA2 | chr12: 66308695- | NA | NA | NA | NA |
| | 66309227 | | | | |
| HMGB1 | chr13: 31035776- | NA | NA | NA | NA |
| | 31035819 | | | | |
| HMGB2 | chr4: 174252527- | NA | NA | NA | NA |
| | 174253230 | | | | |
| HMGCLL1 | NA | 571.064 | −6.558203 | uc003pcp.3 | chr6: 55376873- |
| | | | | | 55378835 |
| HMGN1 | NA | 634.55 | −10.179042 | uc002yxo.3 | chr21: 40714241- |
| | | | | | 40715037 |
| HMGN2 | chr1: 26801640- | NA | NA | NA | NA |
| | 26803133 | | | | |
| HMGN3 | NA | 598.104 | −8.382585 | uc003pit.3 | chr6: 79910962- |
| | | | | | 79911404 |
| HMGN4 | chr6: 26545708- | NA | NA | NA | NA |
| | 26547164 | | | | |
| HMGXB4 | chr22: 35689645- | 1219.36 | −12.421436 | uc003ank.3 | chr22: 35689645- |
| | 35691800 | | | | 35691800 |
| HMHA1 | chr19: 1086006- | NA | NA | NA | NA |
| | 1086627 | | | | |
| HMMR | chr5: 162918171- | NA | NA | NA | NA |
| | 162918953 | | | | |
| HN1 | chr17: 73131344- | 954.565 | −10.996879 | uc002jmz.1 | chr17: 73131344- |
| | 73132196 | | | | 73132196 |
| HNF1B | NA | NA | NA | NA | NA |
| HNRNPA0 | NA | 336.459 | −4.401547 | uc003lbt.3 | chr5: 137087073- |
| | | | | | 137088837 |
| HNRNPC | chr14: 21677296- | 406.165 | −3.654153 | uc010tlr.2 | chr14: 21678927- |
| | 21679381 | | | | 21679495 |
| HNRNPD | chr4: 83275908- | NA | NA | NA | NA |
| | 83276486 | | | | |
| HNRNPH3 | NA | NA | NA | NA | NA |
| HNRNPK | chr9: 86582998- | NA | NA | NA | NA |
| | 86584264 | | | | |
| HNRNPL | NA | 1272.78 | −7.673265 | uc002ojk.3 | chr19: 39327028- |
| | | | | | 39327361 |
| HNRNPLL | NA | NA | NA | NA | NA |
| HNRNPM | chr19: 8553739- | NA | NA | NA | NA |
| | 8554002 | | | | |
| HOMER3 | chr19: 19040010- | NA | NA | NA | NA |
| | 19040251 | | | | |
| HOOK3 | chr8: 42852895- | 825.372 | −7.112012 | uc003xpr.3 | chr8: 42873642- |
| | 42853299 | | | | 42885682 |
| HORMAD1 | NA | 356.501 | −10.053011 | uc001evk.2 | chr1: 150670535- |
| | | | | | 150671129 |
| HORMAD2 | NA | 443.295 | −6.302889 | uc003agy.3 | chr22: 30572157- |
| | | | | | 30573062 |
| HOXA13 | chr7: 27236499- | NA | NA | NA | NA |
| | 27237816 | | | | |
| HOXA2 | NA | 660.524 | −7.729342 | uc003syh.3 | chr7: 27139973- |
| | | | | | 27140344 |
| HOXA4 | NA | NA | NA | NA | NA |
| HOXB2 | NA | 240.319 | −4.111702 | uc002inm.3 | chr17: 46620019- |
| | | | | | 46620429 |
| HOXC11 | NA | NA | NA | NA | NA |
| HOXD10 | chr2: 176983960- | NA | NA | NA | NA |
| | 176984670 | | | | |
| HOXD3 | NA | NA | NA | NA | NA |
| HOXD8 | NA | 813.901 | −9.137874 | uc002ukn.3 | chr2: 176996341- |
| | | | | | 176997423 |
| HOXD9 | chr2: 176988904- | NA | NA | NA | NA |
| | 176989645 | | | | |
| HPGD | NA | NA | NA | NA | NA |
| HPGDS | NA | NA | NA | NA | NA |
| HPRT1 | chrX: 133634108- | NA | NA | NA | NA |
| | 133634698 | | | | |
| HPS3 | NA | 890.305 | −6.052701 | uc021xfk.1 | chr3: 148871440- |
| | | | | | 148871922 |
| HRAS | NA | NA | NA | NA | NA |
| HS2ST1 | NA | 1064.78 | −10.251158 | uc001dmc.4 | chr1: 87563623- |
| | | | | | 87564125 |
| HS3ST3A1 | NA | NA | NA | NA | NA |
| HS3ST3B1 | chr17: 14248964- | NA | NA | NA | NA |
| | 14249492 | | | | |
| HSD17B12 | NA | 395.16 | −5.492512 | uc001mxq.4 | chr11: 43876799- |
| | | | | | 43878169 |
| HSD17B4 | chr5: 118877690- | NA | NA | NA | NA |
| | 118878030 | | | | |
| HSD17B7P2 | chr10: 38647460- | NA | NA | NA | NA |
| | 38647980 | | | | |
| HSF2 | chr6: 122753366- | NA | NA | NA | NA |
| | 122754264 | | | | |
| HSF4 | NA | NA | NA | NA | NA |
| HSF5 | chr17: 56497529- | NA | NA | NA | NA |
| | 56499720 | | | | |
| HSFX2 | NA | 220.966 | −5.912618 | uc022cgm.1 | chrX: 148858449- |
| | | | | | 148858528 |
| HSP90AA1 | chr14: 102547075- | NA | NA | NA | NA |
| | 102548048 | | | | |
| HSPA12A | chr10: 118430703- | NA | NA | NA | NA |
| | 118434291 | | | | |
| HSPA1A | chr6: 31785460- | NA | NA | NA | NA |
| | 31785719 | | | | |
| HSPA1B | chr6: 31797654- | NA | NA | NA | NA |
| | 31798031 | | | | |
| HSPA2 | NA | 1315.15 | −9.278028 | uc001xhk.4 | chr14: 65009488- |
| | | | | | 65009954 |
| HSPA4L | NA | 773.611 | −11.470192 | uc003ifm.3 | chr4: 128754030- |
| | | | | | 128754526 |
| HSPB1 | NA | NA | NA | NA | NA |
| HSPBAP1 | NA | 443.462 | −8.427644 | uc003efv.2 | chr3: 122474107- |
| | | | | | 122474251 |
| HSPG2 | NA | NA | NA | NA | NA |
| HSPH1 | chr13: 31726467- | 510.23 | −4.300559 | uc010aax.1 | chr13: 31726467- |
| | 31726959 | | | | 31726959 |
| HTATIP2 | chr11: 20404752- | 1114.68 | −6.112243 | uc009yia.1 | chr11: 20404752- |
| | 20405329 | | | | 20405329 |
| HTN1 | chr4: 70924297- | 818.794 | −7.951881 | uc003hex.3 | chr4: 70924297- |
| | 70924559 | | | | 70924559 |
| HTR1D | NA | 684.973 | −8.349557 | uc001bgn.3 | chr1: 23518388- |
| | | | | | 23519578 |
| HTR2B | NA | 468.192 | −6.237078 | uc010fxv.3 | chr2: 231972950- |
| | | | | | 231973230 |
| HTR3C | chr3: 183778141- | 442.516 | −5.163575 | uc003fmk.3 | chr3: 183778141- |
| | 183778461 | | | | 183778461 |
| HTR3D | chr3: 183756764- | NA | NA | NA | NA |
| | 183757157 | | | | |
| HTR4 | chr5: 147830595- | 254.357 | −4.476225 | uc021yfg.1 | chr5: 147830595- |
| | 147830835 | | | | 147830835 |
| HTRA4 | NA | NA | NA | NA | NA |
| HUS1B | chr6: 655939- | NA | NA | NA | NA |
| | 656107 | | | | |
| HYAL2 | NA | 449.008 | −6.035312 | uc010hlj.2 | chr3: 50356020- |
| | | | | | 50356332 |
| HYKK | NA | NA | NA | NA | NA |
| HYPK | NA | NA | NA | NA | NA |
| IAH1 | NA | NA | NA | NA | NA |
| IAPP | chr12: 21531361- | NA | NA | NA | NA |
| | 21532914 | | | | |
| ICA1L | NA | NA | NA | NA | NA |
| ICAM1 | chr19: 10395964- | NA | NA | NA | NA |
| | 10397291 | | | | |
| ICAM5 | NA | 1522.82 | −10.821995 | uc002mnu.4 | chr19: 10407293- |
| | | | | | 10407454 |
| ICK | chr6: 52880346- | 586.538 | −8.351957 | uc003pbh.2 | chr6: 52866098- |
| | 52880832 | | | | 52869946 |
| ICMT | NA | NA | NA | NA | NA |
| ICT1 | NA | NA | NA | NA | NA |
| ID2 | NA | NA | NA | NA | NA |
| IDH3A | chr15: 78461348- | 1026.2 | −6.365787 | uc002bdd.3 | chr15: 78461348- |
| | 78462884 | | | | 78462884 |
| IDO2 | NA | NA | NA | NA | NA |
| IER3IP1 | chr18: 44681390- | 1068.51 | −14.262846 | uc002lcu.3 | chr18: 44581390- |
| | 44682547 | | | | 44682547 |
| IFI16 | chr1: 159024692- | NA | NA | NA | NA |
| | 159024945 | | | | |
| IFI35 | NA | 621.206 | −6.403842 | uc021txx.1 | chr17: 41166317- |
| | | | | | 41166476 |
| IFI6 | NA | 602.01 | −4.307831 | uc001bon.1 | chr1: 27992572- |
| | | | | | 27992891 |
| IFIT3 | NA | 458.572 | −8.097483 | uc001kgf.3 | chr10: 91099886- |
| | | | | | 91100725 |
| IFIT5 | NA | 958.445 | −10.549336 | uc010qng.1 | chr10: 91178406- |
| | | | | | 91178576 |
| IFNL1 | chr19: 39789157- | NA | NA | NA | NA |
| | 39789312 | | | | |
| IFRD1 | chr7: 112115575- | NA | NA | NA | NA |
| | 112117258 | | | | |
| IFT172 | chr2: 27694554- | 511.584 | −8.201542 | uc002rku.3 | chr2: 27667240- |
| | 27695041 | | | | 27667298 |
| IFT43 | chr14: 76549921- | NA | NA | NA | NA |
| | 76550092 | | | | |
| IFT52 | NA | NA | NA | NA | NA |
| IFT57 | NA | NA | NA | NA | NA |
| IFT81 | NA | 774.257 | −7.293039 | uc001tqg.3 | chr12: 110605784- |
| | | | | | 110606250 |
| IFT88 | NA | 631.514 | −5.772743 | uc001unh.3 | chr13: 21265315- |
| | | | | | 21265576 |
| IGFBP2 | chr2: 217525615- | NA | NA | NA | NA |
| | 217529156 | | | | |
| IGFBP3 | NA | NA | NA | NA | NA |
| IGFBP7 | NA | NA | NA | NA | NA |
| IGIP | NA | NA | NA | NA | NA |
| IGSF1 | chrX: 130407483- | 356.811 | −3.045307 | uc004ewd.3 | chrX: 130407483- |
| | 130407769 | | | | 130407769 |
| IGSF10 | NA | NA | NA | NA | NA |
| IGSF21 | NA | 989.536 | −8.579008 | uc001bau.2 | chr1: 18704821- |
| | | | | | 18704977 |
| IGSF22 | NA | 651.403 | −10.99586 | uc009yht.2 | chr11: 18725852- |
| | | | | | 18726014 |
| IHH | NA | NA | NA | NA | NA |
| IKBKB | NA | 1763.76 | −18.063942 | uc010lxh.2 | chr8: 42177023- |
| | | | | | 42178362 |
| IKZF1 | chr7: 50468326- | 267.626 | −4.280797 | uc003tov.1 | chr7: 50436069- |
| | 50472798 | | | | 50437142 |
| IL12A | NA | NA | NA | NA | NA |
| IL13 | NA | 510.692 | −4.01612 | uc003kxj.1 | chr5: 131995975- |
| | | | | | 131996801 |
| IL13RA1 | chrX: 117895265- | 879.426 | −6.458981 | uc004eqr.1 | chrX: 117895265- |
| | 117895477 | | | | 117895477 |
| IL15 | NA | NA | NA | NA | NA |
| IL16 | chr15: 81601140- | NA | NA | NA | NA |
| | 81605104 | | | | |
| IL17RD | chr3: 57124010- | NA | NA | NA | NA |
| | 57130420 | | | | |
| IL18R1 | NA | 264.985 | −4.491384 | uc002tbw.4 | chr2: 103013347- |
| | | | | | 103015217 |
| IL1A | chr2: 113531492- | NA | NA | NA | NA |
| | 113532643 | | | | |
| IL1B | NA | NA | NA | NA | NA |
| IL1RAP | chr3: 190366495- | NA | NA | NA | NA |
| | 190369308 | | | | |
| IL2 | NA | 1671.02 | −12.176356 | uc003ier.3 | chr4: 123372626- |
| | | | | | 123372906 |
| IL20RA | NA | 1153.89 | −8.991409 | uc003qhi.3 | chr6: 137321108- |
| | | | | | 137322694 |
| IL24 | NA | 470.072 | −5.163524 | uc001hex.2 | chr1: 207076331- |
| | | | | | 207077484 |
| IL26 | NA | NA | NA | NA | NA |
| IL3 | chr5: 131398485- | NA | NA | NA | NA |
| | 131398896 | | | | |
| IL36G | NA | NA | NA | NA | NA |
| IL5RA | chr3: 3131857- | 2518.7 | −15.762921 | uc010hbp.3 | chr3: 3108008- |
| | 3131926 | | | | 3111908 |
| IL6 | NA | 267.509 | −8.559155 | uc003svj.4 | chr7: 22771193- |
| | | | | | 22771621 |
| IL6ST | chr5: 55230925- | 1532.07 | −5.170405 | NA | NA |
| | 55236909 | | | | |
| IL9 | chr5: 135227935- | 1491.46 | −17.120129 | uc003lbb.1 | chr5: 135227935- |
| | 135228079 | | | | 135228079 |
| ILDR1 | NA | 623.378 | −6.948033 | uc003eeq.3 | chr3: 121706170- |
| | | | | | 121707213 |
| ILDR2 | NA | 312.585 | −4.869887 | uc001gdx.2 | chr1: 166882441- |
| | | | | | 166888591 |
| ILF3 | chr19: 10799989- | 494.774 | −7.045511 | uc002mpn.3 | chr19: 10799989- |
| | 10803095 | | | | 10803095 |
| ILKAP | chr2: 239079043- | NA | NA | NA | NA |
| | 239079176 | | | | |
| ILVBL | NA | 777.432 | −8.338848 | uc002nam.3 | chr19: 15225785- |
| | | | | | 15226062 |
| IMPA2 | NA | NA | NA | NA | NA |
| IMPACT | NA | 572.372 | −7.321587 | uc002kvh.4 | chr18: 22030812- |
| | | | | | 22033494 |
| IMPDH2 | NA | 370827 | −2.846168 | NA | NA |
| IMPG2 | NA | 573.678 | −7.982004 | uc011bhe.2 | chr3: 100941390- |
| | | | | | 100945784 |
| INCENP | NA | 812.149 | −8.57109 | uc001nsw.1 | chr11: 61919449- |
| | | | | | 61920635 |
| ING1 | chr13: 111372280- | NA | NA | NA | NA |
| | 111373421 | | | | |
| ING2 | chr4: 184432106- | NA | NA | NA | NA |
| | 184432249 | | | | |
| ING3 | NA | NA | NA | NA | NA |
| INHBC | chr12: 57843806- | NA | NA | NA | NA |
| | 57844609 | | | | |
| INO80C | NA | NA | NA | NA | NA |
| INO80D | NA | 795.625 | −6.689767 | uc002vaz.4 | chr2: 206858445- |
| | | | | | 206869091 |
| INPP4A | NA | 472.704 | −8.495112 | uc002syx.3 | chr2: 99204072- |
| | | | | | 99207496 |
| INPP4B | chr4: 142949182- | NA | NA | NA | NA |
| | 142949934 | | | | |
| INPP5J | NA | 583.715 | −6.390218 | uc010gwf.3 | chr22: 31523036- |
| | | | | | 31523237 |
| INPP5K | chr17: 1412417- | 490.172 | −6.873754 | uc010cjs.2 | chr17: 1412417- |
| | 1412616 | | | | 1412616 |
| INSL5 | NA | 1192.94 | −11.98178 | uc001dcw.3 | chr1: 67263424- |
| | | | | | 67263695 |
| INSM2 | chr14: 36005160- | NA | NA | NA | NA |
| | 36006260 | | | | |
| INSR | chr19: 7151995- | NA | NA | NA | NA |
| | 7152708 | | | | |
| INSRR | chr1: 156820794- | NA | NA | NA | NA |
| | 156820973 | | | | |
| INTS10 | NA | NA | NA | NA | NA |
| INTS12 | NA | NA | NA | NA | NA |
| INTS3 | NA | 694.649 | −6.241093 | uc009wom.3 | chr1: 153745747- |
| | | | | | 153746555 |
| INTS5 | NA | 417.124 | −4.476812 | uc001nud.3 | chr11: 62414320- |
| | | | | | 62414491 |
| INTS7 | chr1: 212113741- | NA | NA | NA | NA |
| | 212115165 | | | | |
| INTS8 | chr8: 95892463- | NA | NA | NA | NA |
| | 95892721 | | | | |
| INTU | chr4: 128637592- | NA | NA | NA | NA |
| | 128637934 | | | | |
| IP6K1 | NA | 266.267 | −4.174909 | uc021wyl.1 | chr3: 49761728- |
| | | | | | 49764554 |
| IP6K2 | NA | 915.601 | −10.001092 | uc003cup.3 | chr3: 48725436- |
| | | | | | 48725705 |
| IP6K3 | NA | 622.028 | −7.116467 | uc003ofb.2 | chr6: 33689443- |
| | | | | | 33690496 |
| IPCEF1 | NA | 736.322 | −6.535875 | uc003qpv.3 | chr6: 154475618- |
| | | | | | 154480962 |
| IPMK | chr10: 59951278- | NA | NA | NA | NA |
| | 59955836 | | | | |
| IPO13 | NA | NA | NA | NA | NA |
| IPO5 | NA | NA | NA | NA | NA |
| IPO7 | chr11: 9466742- | 890.201 | −9.080962 | uc001mho.3 | chr11: 9466742- |
| | 9469674 | | | | 9469674 |
| IPO9 | NA | NA | NA | NA | NA |
| IQCD | NA | 893.145 | −5.212678 | uc001tuu.3 | chr12: 113633246- |
| | | | | | 113633379 |
| IQCG | chr3: 197615946- | NA | NA | NA | NA |
| | 197616450 | | | | |
| IQCH | chr15: 67599167- | NA | NA | NA | NA |
| | 67599561 | | | | |
| IQCK | chr16: 19867812- | NA | NA | NA | NA |
| | 19868859 | | | | |
| IQGAP2 | chr5: 76003139- | 788.859 | −8.089559 | uc003kek.3 | chr5: 76003139- |
| | 76003957 | | | | 76003957 |
| IRAK4 | NA | 317.106 | −5.56387 | uc001rnt.3 | chr12: 44180519- |
| | | | | | 44183346 |
| IRF2 | chr4: 185308876- | NA | NA | NA | NA |
| | 185309911 | | | | |
| IRF4 | chr6: 401816- | 496.077 | −5.244314 | uc010jne.2 | chr6: 401816- |
| | 402637 | | | | 402637 |
| IRF6 | chr1: 209958968- | 1773.57 | −23.210979 | uc001hhq.2 | chr1: 209958968- |
| | 209961764 | | | | 209961764 |
| IRF9 | NA | 503.706 | −6.24246 | uc001wmq.3 | chr14: 24635406- |
| | | | | | 24635774 |
| IRS2 | NA | NA | NA | NA | NA |
| IRX1 | NA | NA | NA | NA | NA |
| IRX2 | NA | 462.93 | −5.118599 | uc003jda.3 | chr5: 2746279- |
| | | | | | 2746900 |
| IRX3 | chr16: 54317212- | NA | NA | NA | NA |
| | 54317597 | | | | |
| ISL1 | NA | NA | NA | NA | NA |
| ISM1 | NA | NA | NA | NA | NA |
| ISOC1 | chr5: 128448695- | NA | NA | NA | NA |
| | 128449719 | | | | |
| ITFG1 | NA | 437.331 | −19.406737 | uc010vgh.2 | chr16: 47189298- |
| | | | | | 47189678 |
| ITFG3 | NA | 508.634 | −6.578222 | uc002cgf.3 | chr16: 315022- |
| | | | | | 316119 |
| ITGA2 | NA | 829.555 | −9.460145 | uc003joy.3 | chr5: 52386430- |
| | | | | | 52390609 |
| ITGA8 | chr10: 15559088- | NA | NA | NA | NA |
| | 15559156 | | | | |
| ITGA9 | chr3: 37860481- | NA | NA | NA | NA |
| | 37861281 | | | | |
| ITGAE | NA | 463.565 | −6.867557 | uc002fwo.4 | chr17: 3617919- |
| | | | | | 3618140 |
| ITGB1 | chr10: 33189246- | NA | NA | NA | NA |
| | 33190563 | | | | |
| ITGB3BP | chr1: 63919112- | NA | NA | NA | NA |
| | 63919580 | | | | |
| ITIH2 | NA | NA | NA | NA | NA |
| ITIH5 | chr10: 7613368- | NA | NA | NA | NA |
| | 7614284 | | | | |
| ITM2A | NA | NA | NA | NA | NA |
| ITM2C | NA | NA | NA | NA | NA |
| ITPK1 | chr14: 93406068- | NA | NA | NA | NA |
| | 93407905 | | | | |
| ITPKB | NA | 691.288 | −4.875821 | uc010pvo.2 | chr1: 226819391- |
| | | | | | 226822371 |
| ITPR1 | NA | NA | NA | NA | NA |
| IVD | chr15: 40710463- | NA | NA | NA | NA |
| | 40713512 | | | | |
| JADE1 | chr4: 129793418- | NA | NA | NA | NA |
| | 129796379 | | | | |
| JAK2 | chr9: 5126792- | NA | NA | NA | NA |
| | 5128183 | | | | |
| JAKMIP1 | chr4: 6055482- | 650.995 | 0.703103 | uc010ide.3 | chr4: 6055482- |
| | 6055701 | | | | 605S875 |
| JMY | NA | 1269.85 | −7.97121 | uc003kfx.4 | chr5: 78617429- |
| | | | | | 78623038 |
| JPH4 | chr14: 24044160- | NA | NA | NA | NA |
| | 24044790 | | | | |
| JRK | NA | 514.038 | −6.745981 | uc003ywo.3 | chr8: 143738874- |
| | | | | | 143740266 |
| JRKL | chr11: 96125389- | 1183.46 | −9.231227 | uc001pfy.2 | chr11: 96125389- |
| | 96126727 | | | | 96126727 |
| KANSL1L | chr2: 211016129- | 781.669 | −5.641856 | uc002vds.3 | chr2: 210885435- |
| | 211018214 | | | | 210887672 |
| KANSL2 | chr12: 49046995- | NA | NA | NA | NA |
| | 49047826 | | | | |
| KANSL3 | chr2: 97265390- | NA | NA | NA | NA |
| | 97267316 | | | | |
| KARS | NA | 298.054 | −7.248111 | uc002feq.3 | chr16: 75661622- |
| | | | | | 75661792 |
| KAT2B | NA | 203.263 | −4.50214 | uc003cbq.3 | chr3: 20194018- |
| | | | | | 20195896 |
| KAT7 | chr17: 47904865- | NA | NA | NA | NA |
| | 47906458 | | | | |
| KATNBL1 | NA | 639.961 | −5.083765 | uc010ubz.2 | chr15: 34438271- |
| | | | | | 34439396 |
| KAZN | NA | 302.345 | −4.782558 | uc009vog.1 | chr1: 15392294- |
| | | | | | 15394651 |
| KBTBD2 | NA | 254.75 | −4.238367 | uc003tdb.2 | chr7: 32907778- |
| | | | | | 32908956 |
| KBTBD4 | chr11: 47593749- | NA | NA | NA | NA |
| | 47594481 | | | | |
| KCNC1 | NA | 739.61 | −7.527893 | uc009yhc.1 | chr11: 17803282- |
| | | | | | 17804602 |
| KCND1 | NA | 337.843 | −3.117938 | uc004dlw.1 | chrX: 48818639- |
| | | | | | 48819841 |
| KCNG3 | NA | NA | NA | NA | NA |
| KCNH5 | chr14: 63173945- | 828.962 | −6.455136 | uc001xfz.1 | chr14: 63178958- |
| | 63174225 | | | | 63179404 |
| KCNH7 | NA | 345.963 | −7.000413 | uc002uch.2 | chr2: 163227917- |
| | | | | | 163228338 |
| KCNJ13 | chr2: 233630512- | NA | NA | NA | NA |
| | 233632900 | | | | |
| KCNK10 | chr14: 88646452- | NA | NA | NA | NA |
| | 88651878 | | | | |
| KCNK12 | chr2: 47747915- | NA | NA | NA | NA |
| | 47748045 | | | | |
| KCNK17 | NA | 574.435 | −9.506581 | uc003ooo.3 | chr6: 39266777- |
| | | | | | 39267202 |
| KGNK6 | chr19: 38818044- | NA | NA | NA | NA |
| | 38819649 | | | | |
| KCNK7 | NA | 494.258 | −7.431088 | uc001oes.3 | chr11: 65360326- |
| | | | | | 65360475 |
| KCNMB2 | NA | 670.3 | −10.136079 | uc003fjd.3 | chr3: 178560726- |
| | | | | | 178562217 |
| KCNRG | chr13: 50594591- | NA | NA | NA | NA |
| | 50595058 | | | | |
| KCNS1 | NA | NA | NA | NA | NA |
| KCNS3 | chr2: 18113752- | NA | NA | NA | NA |
| | 18114225 | | | | |
| KCNT2 | NA | NA | NA | NA | NA |
| KCNU1 | chr8: 36793439- | NA | NA | NA | NA |
| | 36793643 | | | | |
| KCTD1 | NA | 1236.43 | −5.917729 | uc002kvy.3 | chr18: 24035684- |
| | | | | | 24039577 |
| KCTD18 | chr2: 201353684- | NA | NA | NA | NA |
| | 201354822 | | | | |
| KCTD20 | NA | 987.214 | −6.398113 | uc003ome.3 | chr6: 36454953- |
| | | | | | 36458315 |
| KCTD5 | NA | 749.925 | −10.117853 | uc002crd.3 | chr16: 2757329- |
| | | | | | 2759031 |
| KCTD6 | chr3: 58487360- | NA | NA | NA | NA |
| | 58488087 | | | | |
| KCTD7 | chr7: 66105382- | 838.931 | −3.75676 | uc003tvd.4 | chr7: 66105382- |
| | 66108216 | | | | 66108216 |
| KDELR2 | NA | 931.318 | −6.616868 | uc003sqf.4 | chr7: 6500712- |
| | | | | | 6502596 |
| KDM1A | NA | 434.469 | −4.849513 | uc001bgi.2 | chr1: 23409858- |
| | | | | | 23410184 |
| KDM4E | chr11: 94760243- | NA | NA | NA | NA |
| | 94760760 | | | | |
| KDM5A | NA | 659.067 | −4.563325 | uc021qsr.1 | chr12: 389223- |
| | | | | | 394828 |
| KDM5D | chrY: 21867301- | 906.923 | −7.61484 | uc004fuf.3 | chrY: 21867301- |
| | 21867880 | | | | 21867880 |
| KEAP1 | chr19: 10596796- | 459.359 | −4.392235 | uc002mop.1 | chr19: 10596796- |
| | 10597327 | | | | 10597327 |
| KEL | chr7: 142638201- | NA | NA | NA | NA |
| | 142638338 | | | | |
| KHDC1 | NA | NA | NA | NA | NA |
| KHDC1L | chr6: 73933268- | NA | NA | NA | NA |
| | 73933470 | | | | |
| KHDRBS2 | NA | NA | NA | NA | NA |
| KIAA0100 | NA | 1072.18 | −8.413183 | uc002hbv.3 | chr17: 26966079- |
| | | | | | 26966322 |
| KIAA0101 | NA | 504.02 | −7.427867 | uc002ank.3 | chr15: 64657211- |
| | | | | | 64658228 |
| KIAA0196 | NA | 390.205 | −5.550454 | uc003yrt.3 | chr8: 126036503- |
| | | | | | 126036858 |
| KIAA0226 | chr3: 197398259- | 415.523 | −4.350569 | uc003fye.1 | chr3: 197403088- |
| | 197401888 | | | | 197403710 |
| KIAA0368 | NA | NA | NA | NA | NA |
| KIAA0408 | chr6: 127759551- | NA | NA | NA | NA |
| | 127765253 | | | | |
| KIAA0513 | NA | 328.329 | −4.435821 | uc002fit.3 | chr16: 85112614- |
| | | | | | 85113362 |
| KIAA0586 | chr14: 59014651- | NA | NA | NA | NA |
| | 59015549 | | | | |
| KIAA0754 | NA | 1153.47 | −10.839432 | uc009vvt.1 | chr1: 39880222- |
| | | | | | 39882154 |
| KIAA0825 | NA | 1393.77 | −13.900212 | uc003kkn.3 | chr5: 93486556- |
| | | | | | 93489711 |
| KIAA0895 | chr7: 36394805- | NA | NA | NA | NA |
| | 36396423 | | | | |
| KIAA0922 | NA | 1119.82 | −2.964158 | uc003inm.4 | chr4: 154557729- |
| | | | | | 154557862 |
| KIAA1024 | chr15: 79760727- | NA | NA | NA | NA |
| | 79764642 | | | | |
| KIAA1107 | NA | 437.567 | −4.711478 | uc010otd.2 | chr1: 92649888- |
| | | | | | 92650280 |
| KIAA1143 | NA | 657.511 | −9.698425 | uc011bac.2 | chr3: 44790236- |
| | | | | | 44794832 |
| KIAA1147 | NA | 309.127 | −5.924985 | uc003vwk.3 | chr7: 141356528- |
| | | | | | 141362455 |
| KIAA1191 | chr5: 175773066- | NA | NA | NA | NA |
| | 175774602 | | | | |
| KIAA1210 | chrX: 118212598- | NA | NA | NA | NA |
| | 118215291 | | | | |
| KIAA1211 | NA | 1150.72 | −11.027512 | uc003hbk.2 | chr4: 57193971- |
| | | | | | 57196890 |
| KIAA1217 | NA | NA | NA | NA | NA |
| KIAA1257 | chr3: 128689782- | NA | NA | NA | NA |
| | 128690067 | | | | |
| KIAA1324 | NA | 659.889 | −5.878843 | uc009wey.3 | chr1: 109745635- |
| | | | | | 109749403 |
| KIAA1328 | chr18: 34465680- | 616.056 | −5.582211 | uc002kzz.3 | chr18: 34802191- |
| | 34466367 | | | | 34805288 |
| KIAA1407 | NA | 1381.9 | −7.438897 | uc003eax.3 | chr3: 113682984- |
| | | | | | 113684001 |
| KIAA1429 | chr8: 95500005- | NA | NA | NA | NA |
| | 95500933 | | | | |
| KIAA1462 | chr10: 30301729- | NA | NA | NA | NA |
| | 30306811 | | | | |
| KIAA1468 | NA | 304.691 | −4.340257 | uc002lil.3 | chr18: 59972768- |
| | | | | | 59974355 |
| KIAA1551 | chr12: 32145470- | 564.85 | −7.764018 | uc001rks.3 | chr12: 32145470- |
| | 32146043 | | | | 32146043 |
| KIAA1586 | chr6: 56919662- | 1080.08 | −13.340759 | uc003pdj.3 | chr6: 56919662- |
| | 56920023 | | | | 56920023 |
| KIAA1598 | chr10: 118615739- | NA | NA | NA | NA |
| | 118616492 | | | | |
| KIAA1715 | NA | 494.356 | −5.23998 | uc002ukc.1 | chr2: 176790410- |
| | | | | | 176794694 |
| KIAA1755 | NA | 672.605 | −4.753566 | uc002xhv.1 | chr20: 36838907- |
| | | | | | 36841443 |
| KIAA1841 | chr2: 61315656- | NA | NA | NA | NA |
| | 61316639 | | | | |
| KIAA1875 | NA | NA | NA | NA | NA |
| KIAA2013 | NA | NA | NA | NA | NA |
| KIAA2018 | NA | 334.653 | −6.928917 | uc003eal.3 | chr3: 113367233- |
| | | | | | 113373790 |
| KIAA2022 | chrX: 73952691- | 498.153 | −5.995081 | uc004eby.3 | chrX: 73952691- |
| | 73959239 | | | | 73959239 |
| KIF11 | NA | NA | NA | NA | NA |
| KIF15 | NA | 1067.06 | −7.175229 | uc003cnx.4 | chr3: 44894228- |
| | | | | | 44894748 |
| KIF16B | NA | 613.886 | −8.785953 | uc010gci.2 | chr20: 16347488- |
| | | | | | 16347790 |
| KIF17 | NA | NA | NA | NA | NA |
| KIF20B | chr10: 91533806- | NA | NA | NA | NA |
| | 91534700 | | | | |
| KIF23 | NA | NA | NA | NA | NA |
| KIF25 | NA | 603.823 | −6.747937 | uc003qwk.1 | chr6: 168445677- |
| | | | | | 168445769 |
| KIF26B | NA | NA | NA | NA | NA |
| KIF2B | chr17: 51902417- | NA | NA | NA | NA |
| | 51902573 | | | | |
| KIF5A | NA | NA | NA | NA | NA |
| KIFC1 | NA | 1253.31 | −22.622704 | uc003oef.4 | chr6: 33377468- |
| | | | | | 33377699 |
| KIN | NA | 1740.53 | −12.832908 | uc010qba.2 | chr10: 7801234- |
| | | | | | 7801786 |
| KIR2DL5B | NA | 439.74 | −5.622688 | uc002quk.1 | chr19_g1000209_random: |
| | | | | | 95743-95246 |
| KIR3DX1 | NA | 358.231 | −6.46399 | uc010erm.2 | chr19: 55054792- |
| | | | | | 55055195 |
| KISS1R | NA | 754.272 | −9.014252 | uc002lqk.4 | chr19: 920749- |
| | | | | | 921015 |
| KITLG | chr12: 88886570- | 473.93 | −8.583759 | NA | NA |
| | 88890888 | | | | |
| KL | NA | 592.971 | −11.566181 | uc001uus.3 | chr13: 33538324- |
| | | | | | 33640282 |
| KLF3 | NA | 230.992 | −3.584488 | uc003gtg.2 | chr4: 38691505- |
| | | | | | 38692372 |
| KLF4 | chr9: 110247133- | NA | NA | NA | NA |
| | 110248031 | | | | |
| KLF8 | chrX: 56292186- | 808.997 | −7.819798 | uc004dur.3 | chrX: 56310928- |
| | 56295922 | | | | 56314322 |
| KLHDC1 | NA | NA | NA | NA | NA |
| KLHDC10 | NA | 535.154 | −6.114609 | uc003vpj.2 | chr7: 129770587- |
| | | | | | 129775560 |
| KLHDG8B | NA | 1531.55 | −9.918008 | uc003cwh.3 | chr3: 49213235- |
| | | | | | 49213919 |
| KLHL1 | chr13: 70274725- | NA | NA | NA | NA |
| | 70275833 | | | | |
| KLHL10 | NA | 1014.89 | −9.454426 | NA | NA |
| KLHL12 | NA | 953.125 | −13.214116 | uc001gym.1 | chr1: 202860230- |
| | | | | | 202861764 |
| KLHL2 | NA | 561.619 | −7.030205 | uc003irb.3 | chr4: 166243213- |
| | | | | | 166244308 |
| KLHL20 | NA | 995.589 | −9.69386 | uc001gjc.3 | chr1: 173754386- |
| | | | | | 173755840 |
| KLHL24 | NA | 666.037 | −9.139646 | uc003flv.3 | chr3: 183397075- |
| | | | | | 183402304 |
| KLHL25 | NA | 410.832 | −5.091727 | uc002bly.3 | chr15: 85302559- |
| | | | | | 85304242 |
| KLHL3 | chr5: 136996400- | NA | NA | NA | NA |
| | 136997588 | | | | |
| KLHL32 | chr6: 97562445- | NA | NA | NA | NA |
| | 97562618 | | | | |
| KLHL36 | NA | 242.172 | −4.975672 | uc002fig.3 | chr16: 84695740- |
| | | | | | 84695916 |
| KLHL41 | NA | 702.892 | −6.305672 | uc010zdh.1 | chr2: 170382207- |
| | | | | | 170382772 |
| KLHL6 | NA | 426.339 | −7.349544 | uc003flt.1 | chr3: 183208894- |
| | | | | | 183211994 |
| KLK10 | NA | NA | NA | NA | NA |
| KLK11 | chr19: 51525487- | NA | NA | NA | NA |
| | 51525800 | | | | |
| KLK12 | chr19: 51532348- | NA | NA | NA | NA |
| | 51532708 | | | | |
| KLRC2 | chr12: 10579193- | 571.218 | −6.036966 | uc001qyk.2 | chr12: 10583206- |
| | 10579474 | | | | 10583715 |
| KLRC4 | NA | 969.978 | −9.978313 | uc001qye.3 | chr12: 10559983- |
| | | | | | 10560251 |
| KLRD1 | NA | NA | NA | NA | NA |
| KLRG2 | chr7: 139138088- | 659.267 | −6.44214 | uc003wb.3 | chr7: 139138088- |
| | 139138332 | | | | 139138332 |
| KMT2C | NA | NA | NA | NA | NA |
| KMT2E | chr7: 104731870- | NA | NA | NA | NA |
| | 104732057 | | | | |
| KNG1 | NA | NA | NA | NA | NA |
| KPRP | chr1: 152733805- | NA | NA | NA | NA |
| | 152734529 | | | | |
| KRAS | NA | NA | NA | NA | NA |
| KRBA2 | chr17: 8271973- | NA | NA | NA | NA |
| | 8272451 | | | | |
| KREMEN1 | NA | 367.93 | −7.653458 | uc003ael.3 | chr22: 29538101- |
| | | | | | 29542808 |
| KRIT1 | chr7: 91828283- | NA | NA | NA | NA |
| | 91830049 | | | | |
| KRT1 | chr12: 53068520- | NA | NA | NA | NA |
| | 53068976 | | | | |
| KRT12 | chr17: 39017430- | NA | NA | NA | NA |
| | 39017912 | | | | |
| KRT23 | chr17: 39081413- | 1044.64 | −9.789248 | uc002hvm.1 | chr17: 39078952- |
| | 39081601 | | | | 39079240 |
| KRT28 | NA | 877.535 | −11.130376 | uc002hvh.1 | chr17: 38948448- |
| | | | | | 38948678 |
| KRT73 | NA | NA | NA | NA | NA |
| KRTCAP2 | NA | NA | NA | NA | NA |
| KSR1 | NA | 548.082 | −5.264453 | uc002gzm.3 | chr17: 25948962- |
| | | | | | 25950718 |
| KYNU | NA | NA | NA | NA | NA |
| L3HYPDH | chr14: 59939406- | NA | NA | NA | NA |
| | 59939682 | | | | |
| LACC1 | chr13: 44465585- | NA | NA | NA | NA |
| | 44468068 | | | | |
| LACE1 | chr6: 108843629- | NA | NA | NA | NA |
| | 108844251 | | | | |
| LACTB2 | chr8: 71549501- | NA | NA | NA | NA |
| | 71550094 | | | | |
| LAG3 | NA | NA | NA | NA | NA |
| LALBA | NA | NA | NA | NA | NA |
| LAMA2 | chr6: 129837493- | NA | NA | NA | NA |
| | 129837710 | | | | |
| LAMA5 | NA | 469.431 | −4.589171 | uc002ycq.3 | chr20: 60884121- |
| | | | | | 60884391 |
| LAMB4 | NA | NA | NA | NA | NA |
| LAMP2 | chrX: 119560003- | NA | NA | NA | NA |
| | 119562338 | | | | |
| LAMTOR3 | chr4: 100799495- | NA | NA | NA | NA |
| | 100803150 | | | | |
| LAPTM4A | NA | NA | NA | NA | NA |
| LARP1B | NA | 1009.79 | −4.191284 | uc003ifx.3 | chr4: 129028558- |
| | | | | | 129030056 |
| LARP6 | chr15: 71123889- | NA | NA | NA | NA |
| | 71124390 | | | | |
| LARS | chr5: 145492589- | 368.341 | −4.553691 | uc003lnw.1 | chr5: 145492589- |
| | 145493668 | | | | 145493668 |
| LASP1 | chr17: 37055079- | NA | NA | NA | NA |
| | 37055453 | | | | |
| LATS1 | chr6: 150004055- | NA | NA | NA | NA |
| | 150004151 | | | | |
| LBR | chr1: 225589204- | 591.618 | −6.773939 | uc001hoy.3 | chr1: 225589204- |
| | 225591004 | | | | 225591004 |
| LCA5 | chr6: 80222489- | 803.905 | −7.838223 | uc011dyr.2 | chr6: 80222489- |
| | 80222856 | | | | 80222856 |
| LCE1B | chr1: 152785280- | NA | NA | NA | NA |
| | 152785585 | | | | |
| LCE1E | chr1: 152760133- | NA | NA | NA | NA |
| | 152760901 | | | | |
| LCE2A | NA | NA | NA | NA | NA |
| LCE3D | NA | 526.507 | −5.216407 | uc001fab.3 | chr1: 152551860- |
| | | | | | 152552133 |
| LCLAT1 | chr2: 30829067- | NA | NA | NA | NA |
| | 30830712 | | | | |
| LCN12 | chr9: 139849226- | NA | NA | NA | NA |
| | 139849949 | | | | |
| LCOR | chr10: 98718176- | NA | NA | NA | NA |
| | 98724198 | | | | |
| LCORL | chr4: 17844839- | 655.71 | −9.487061 | uc021xmq.1 | chr4: 17844839- |
| | 17847343 | | | | 17847343 |
| LDB1 | chr10: 103868182- | NA | NA | NA | NA |
| | 103868731 | | | | |
| LDLRAD3 | NA | NA | NA | NA | NA |
| LDLRAD4 | NA | 798.024 | −5.634334 | uc002ksh.1 | chr18: 13645657- |
| | | | | | 13652753 |
| LDOC1 | chrX: 140269931- | NA | NA | NA | NA |
| | 140270765 | | | | |
| LEAP2 | NA | 684.928 | −7.479592 | uc003kyc.3 | chr5: 132210139- |
| | | | | | 132210582 |
| LEFTY1 | NA | NA | NA | NA | NA |
| LEKR1 | NA | 359.476 | −6.017368 | uc021xgh.1 | chr3: 156763540- |
| | | | | | 156763918 |
| LEMD1 | NA | 1548.09 | −1.964069 | uc001hci.2 | chr1: 205350506- |
| | | | | | 205350862 |
| LEMD3 | NA | NA | NA | NA | NA |
| LEO1 | NA | NA | NA | NA | NA |
| LEPROT | NA | 456.844 | −7.809576 | uc009wao.3 | chr1: 65893487- |
| | | | | | 65893692 |
| LEPROTL1 | chr8: 29963379- | NA | NA | NA | NA |
| | 29965587 | | | | |
| LETM1 | NA | 605.562 | −4.578215 | uc003gdv.3 | chr4: 1813206- |
| | | | | | 1816150 |
| LETMD1 | chr12: 51450387- | 1227.37 | −1.655602 | uc010sna.2 | chr12: 51450387- |
| | 51450735 | | | | 51450735 |
| LGALS13 | NA | 624.97 | −7.722091 | uc002omb.3 | chr19: 40097980- |
| | | | | | 40098114 |
| LGALS3 | NA | NA | NA | NA | NA |
| LGALS8 | chr1: 236711462- | NA | NA | NA | NA |
| | 236716279 | | | | |
| LGI4 | chr19: 35615417- | NA | NA | NA | NA |
| | 35616096 | | | | |
| LHFP | NA | NA | NA | NA | NA |
| LHX8 | NA | 289.899 | −6.537418 | uc001dgo.3 | chr1: 75626581- |
| | | | | | 75627218 |
| LHX9 | chr1: 197898390- | NA | NA | NA | NA |
| | 197899273 | | | | |
| LIG1 | NA | 559.471 | −6.726625 | uc010xze.1 | chr19: 48618703- |
| | | | | | 48618905 |
| LIG3 | chr17: 33330068- | 381.161 | −5.376514 | uc002hik.2 | chr17: 33331526- |
| | 33330191 | | | | 33332088 |
| LILRA1 | chr19: 55110760- | NA | NA | NA | NA |
| | 55111185 | | | | |
| LILRA2 | chr19: 55098668- | 1349.04 | −1.240736 | uc010ern.3 | chr19: 55098668- |
| | 55099027 | | | | 55099027 |
| LILRB2 | NA | NA | NA | NA | NA |
| LIMA1 | NA | NA | NA | NA | NA |
| LIMCH1 | NA | NA | NA | NA | NA |
| LIMK1 | NA | 475.634 | −5.890821 | uc003uac.1 | chr7: 73523496- |
| | | | | | 73525770 |
| LIMS1 | chr2: 109300456- | 524.368 | −5.43496 | uc002tel.3 | chr2: 109300456- |
| | 109303702 | | | | 109303702 |
| LIN28A | chr1: 26752950- | 399.569 | −3.142143 | uc001bmj.3 | chr1: 26752950- |
| | 26756219 | | | | 26756219 |
| LIN7B | NA | 1456.95 | −11.73772 | uc002pmp.3 | chr19: 49621631- |
| | | | | | 49621717 |
| LIN7C | NA | NA | NA | NA | NA |
| LINC00167 | NA | 609.083 | −5.315011 | uc009zcu.2 | chr11: 129873393- |
| | | | | | 129875381 |
| LINC00305 | chr18: 61747243- | NA | NA | NA | NA |
| | 61747535 | | | | |
| LING00493 | chr20: 18550050- | NA | NA | NA | NA |
| | 18550203 | | | | |
| LINC00657 | NA | 363.928 | −6.379406 | uc002xet.3 | chr20: 34633540- |
| | | | | | 34638313 |
| LINC00893 | NA | NA | NA | NA | NA |
| LINC00937 | chr12: 8509560- | 300.316 | −4.682225 | NA | NA |
| | 8509621 | | | | |
| LINC00998 | chr7: 112756773- | NA | NA | NA | NA |
| | 112757547 | | | | |
| LINC01225 | NA | NA | NA | NA | NA |
| LINC01387 | NA | NA | NA | NA | NA |
| LINC01559 | NA | 264 | −5.837604 | uc001rbs.2 | chr12: 13523605- |
| | | | | | 13526137 |
| LINC01588 | chr14: 50448430- | NA | NA | NA | NA |
| | 50449022 | | | | |
| LIPF | chr10: 90428518- | 875.717 | −8.686488 | uc001kfg.2 | chr10: 90438439- |
| | 90429295 | | | | 90438572 |
| LIX1L | NA | NA | NA | NA | NA |
| LMAN1 | chr18: 56995056- | 352.317 | −8.665676 | uc002lhz.3 | chr18: 56995056- |
| | 56998324 | | | | 56998324 |
| LMAN2 | chr5: 176758563- | NA | NA | NA | NA |
| | 176759086 | | | | |
| LMAN2 | NA | 394.844 | −5.421883 | uc002swu.3 | chr2: 97371667- |
| | | | | | 97372992 |
| LMBR1 | NA | NA | NA | NA | NA |
| LMBR1L | NA | 276.116 | −6.40512 | uc001rtg.4 | chr12: 49490923- |
| | | | | | 49491454 |
| LMBRD1 | NA | 1497.52 | −7.558011 | uc003pez.3 | chr6: 70385641- |
| | | | | | 70386049 |
| LMLN | chr3: 197765539- | 906.081 | −10.437612 | uc003fyt.3 | chr3: 197765539- |
| | 197770591 | | | | 197770591 |
| LMNB1 | NA | 717.124 | −5.990875 | uc003kud.2 | chr5: 126171957- |
| | | | | | 126172712 |
| LMNTD1 | NA | NA | NA | NA | NA |
| LMO3 | NA | 746.338 | −8.882417 | uc001rdj.2 | chr12: 16701306- |
| | | | | | 16704155 |
| LMO7 | chr13: 76432080- | NA | NA | NA | NA |
| | 76434006 | | | | |
| LNP1 | chr3: 100174771- | 643.998 | −7.662825 | uc003dtx.4 | chr3: 100174771- |
| | 100175170 | | | | 100175170 |
| LNX2 | chr13: 28120050- | NA | NA | NA | NA |
| | 28122471 | | | | |
| LONRF2 | chr2: 100889753- | NA | NA | NA | NA |
| | 100900759 | | | | |
| LPAR2 | chr19: 19734466- | NA | NA | NA | NA |
| | 19735064 | | | | |
| LPAR4 | chrX: 78011480- | NA | NA | NA | NA |
| | 78012578 | | | | |
| LPCAT4 | chr15: 34651089- | 1019.46 | −8.64513 | uc001zig.3 | chr15: 34651089- |
| | 34651327 | | | | 34651327 |
| LPP | NA | NA | NA | NA | NA |
| LPPR1 | NA | 332.321 | −9.93722 | uc004bbb.3 | chr9: 104086340- |
| | | | | | 104087417 |
| LPPR5 | NA | NA | NA | NA | NA |
| LRAT | chr4: 155670289- | 1302.97 | −14.460577 | uc003iom.1 | chr4: 155670289- |
| | 155674270 | | | | 155674270 |
| LRBA | chr4: 151185811- | NA | NA | NA | NA |
| | 151186873 | | | | |
| LRCOL1 | NA | NA | NA | NA | NA |
| LRMP | NA | 612.372 | −10.643201 | uc001rgh.3 | chr12: 25260995- |
| | | | | | 25261269 |
| LRP11 | chr6: 150139894- | NA | NA | NA | NA |
| | 150141684 | | | | |
| LRP12 | chr8: 105501459- | NA | NA | NA | NA |
| | 105502900 | | | | |
| LRP1B | NA | NA | NA | NA | NA |
| LRP2 | NA | 965.885 | −13.031261 | uc010zdf.1 | chr2: 170097260- |
| | | | | | 170097453 |
| LRP2BP | chr4: 186285032- | 1133 | −7.588672 | uc003ixj.2 | chr4: 186285032- |
| | 186288333 | | | | 186288333 |
| LRP4 | chr11: 46878268- | NA | NA | NA | NA |
| | 46880533 | | | | |
| LRPPRC | chr2: 44113363- | NA | NA | NA | NA |
| | 44115738 | | | | |
| LRR1 | chr14: 50081133- | 427.47 | −9.916191 | uc001wwn.3 | chr14: 50081215- |
| | 50081390 | | | | 50081390 |
| LRRC10 | NA | 586.796 | −7.437086 | uc001svc.3 | chr12: 70002345- |
| | | | | | 70003784 |
| LRRC2 | chr3: 46556878- | NA | NA | NA | NA |
| | 46560503 | | | | |
| LRRC23 | NA | 541.167 | −6.066948 | uc001qrp.3 | chr12: 7023055- |
| | | | | | 7023406 |
| LRRC27 | chr10: 134166410- | 755.009 | −5.70302 | uc010quv.1 | chr10: 134166465- |
| | 134166901 | | | | 134166901 |
| LRRC3 | chr21: 45877302- | NA | NA | NA | NA |
| | 45878739 | | | | |
| LRRC34 | NA | NA | NA | NA | NA |
| LRRC36 | chr16: 67418993- | 590.496 | −2.319103 | uc002esv.3 | chr16: 67418993- |
| | 67419109 | | | | 67419109 |
| LRRC37A4P | NA | 814.737 | −7.342178 | uc010wyl.2 | chr17_ctg5_hap1: |
| | | | | | 1489501-1489848 |
| LRRC4 | NA | NA | NA | NA | NA |
| LRRC40 | chr1: 70610485- | 723.331 | −7.055763 | uc001der.2 | chr1: 70610485- |
| | 70611482 | | | | 70611482 |
| LRRC46 | NA | NA | NA | NA | NA |
| LRRC47 | chr1: 3696784- | NA | NA | NA | NA |
| | 3697651 | | | | |
| LRRC59 | chr17: 48458594- | NA | NA | NA | NA |
| | 48460348 | | | | |
| LRRC61 | NA | NA | NA | NA | NA |
| LRRC66 | NA | NA | NA | NA | NA |
| LRRC69 | NA | 348.083 | −4.421226 | uc003yev.1 | chr8: 92231229- |
| | | | | | 92231464 |
| LRRFIP2 | NA | NA | NA | NA | NA |
| LRRIQ1 | NA | 552.454 | −13.393013 | uc001tac.3 | chr12: 85638720- |
| | | | | | 85638883 |
| LRRK2 | chr12: 40758936- | NA | NA | NA | NA |
| | 40763086 | | | | |
| LRRN1 | chr3: 3888477- | NA | NA | NA | NA |
| | 3889387 | | | | |
| LRRTM1 | NA | 824.091 | −6.058837 | uc002sok.1 | chr2: 80529003- |
| | | | | | 80529375 |
| LRRTM2 | NA | 703.474 | −7.341314 | uc011cyz.1 | chr5: 138205079- |
| | | | | | 138208698 |
| LRTM1 | NA | NA | NA | NA | NA |
| LSM6 | chr4: 147110809- | NA | NA | NA | NA |
| | 147111213 | | | | |
| LSM7 | NA | NA | NA | NA | NA |
| LSMEM2 | NA | NA | NA | NA | NA |
| LSR | NA | NA | NA | NA | NA |
| LST1 | NA | NA | NA | NA | NA |
| LTV1 | NA | NA | NA | NA | NA |
| LUC7L2 | NA | 736.939 | −8.796084 | uc003vuz.1 | chr7: 139097331- |
| | | | | | 139097571 |
| LUM | chr12: 91497232- | 797.205 | −8.951197 | uc001tbm.3 | chr12: 91497232- |
| | 91497941 | | | | 91497941 |
| LURAP1 | NA | NA | NA | NA | NA |
| LURAP1L | NA | 1528.87 | −20.319084 | uc003zkw.3 | chr9: 12821760- |
| | | | | | 12823059 |
| LUZP1 | NA | NA | NA | NA | NA |
| LUZP4 | NA | NA | NA | NA | NA |
| LVRN | NA | 551.84 | −6.335321 | uc003kro.3 | chr5: 115361816- |
| | | | | | 115363299 |
| LY6K | chr8: 143784790- | 518.871 | −5.783976 | uc011ljw.2 | chr8: 143784408- |
| | 143785584 | | | | 143785584 |
| LY75 | chr2: 160659868- | 216.154 | −6.508649 | uc002ubc.4 | chr2: 160659868- |
| | 160661554 | | | | 160661554 |
| LYAR | NA | NA | NA | NA | NA |
| LYPD6 | NA | 405.439 | −7.780598 | uc021vqt.1 | chr2: 150327365- |
| | | | | | 150330659 |
| LYRM4 | chr6: 5108653- | 453.051 | −3.240128 | uc003mwp.3 | chr6: 5108653- |
| | 5109656 | | | | 5109656 |
| LYRM7 | chr5: 130535295- | 239.038 | −3.82979 | uc003kvg.1 | chr5: 130535295- |
| | 130541119 | | | | 130541119 |
| LYSMD3 | NA | 261.745 | −8.124103 | uc010jaz.2 | chr5: 89811445- |
| | | | | | 89814628 |
| LYZ | chr12: 69747000- | NA | NA | NA | NA |
| | 69748013 | | | | |
| LYZL6 | NA | 635.989 | −5.71832 | uc002hkj.2 | chr17: 34261523- |
| | | | | | 34261799 |
| LZIC | chr1: 9989776- | NA | NA | NA | NA |
| | 9990456 | | | | |
| MAATS1 | NA | 527.697 | −6.838046 | uc010hqx.1 | chr3: 119428747- |
| | | | | | 119429208 |
| MACC1 | chr7: 20174279- | NA | NA | NA | NA |
| | 20180568 | | | | |
| MACF1 | chr1: 39748051- | 518.011 | −3.777244 | uc021olt.1 | chr1: 39951467- |
| | 39748740 | | | | 39952810 |
| MAD2L1BP | NA | 385.753 | −4.580107 | uc003ovu.3 | chr6: 43608271- |
| | | | | | 43608688 |
| MAEL | NA | 1167.35 | −7.297603 | uc021peh.1 | chr1: 166991093- |
| | | | | | 166991447 |
| MAF | NA | NA | NA | NA | NA |
| MAGEA10 | NA | NA | NA | NA | NA |
| MAGEB1 | chrX: 30269655- | NA | NA | NA | NA |
| | 30270155 | | | | |
| MAGEB18 | chrX: 26158297- | NA | NA | NA | NA |
| | 26158853 | | | | |
| MAGEB2 | NA | 468.812 | −4.840821 | uc004dbz.3 | chrX: 30237658- |
| | | | | | 30238206 |
| MAGED1 | NA | NA | NA | NA | NA |
| MAGEE1 | NA | 1027.39 | −13.295172 | uc004ecm.2 | chrX: 75651198- |
| | | | | | 75651746 |
| MAGI3 | NA | 476.677 | −4.31365 | uc001edk.3 | chr1: 114226637- |
| | | | | | 114228545 |
| MAGOH | NA | NA | NA | NA | NA |
| MAGOHB | chr12: 10756364- | NA | NA | NA | NA |
| | 10758873 | | | | |
| MAK16 | NA | 907.391 | −9.988265 | uc003xjj.3 | chr8: 33356148- |
| | | | | | 33358778 |
| MAL2 | NA | 724.863 | −4.938722 | uc003yop.3 | chr8: 120255729- |
| | | | | | 120257914 |
| MALT1 | NA | 787.359 | −8.554715 | uc002lhm.1 | chr18: 56415075- |
| | | | | | 56417370 |
| MAN1A2 | chr1: 118065580- | NA | NA | NA | NA |
| | 118068320 | | | | |
| MAN1B1 | chr9: 140003044- | NA | NA | NA | NA |
| | 140003639 | | | | |
| MAN2A1 | chr5: 109202700- | 1262.91 | −6.52922 | uc003kou.1 | chr5: 109202700- |
| | 109203429 | | | | 109203429 |
| MANEA | NA | NA | NA | NA | NA |
| MANSC4 | NA | 845.402 | −7.06184 | uc010sjs.1 | chr12: 27915599- |
| | | | | | 27915670 |
| MAOB | NA | 643.085 | −6.289381 | uc011mkx.2 | chrX: 43625857- |
| | | | | | 43626865 |
| MAP1LC3C | NA | 310.961 | −5.492857 | uc001hzk.2 | chr1: 242158792- |
| | | | | | 242159464 |
| MAP2 | chr2: 210595122- | NA | NA | NA | NA |
| | 210598834 | | | | |
| MAP2K6 | NA | 361.476 | −6.596816 | uc002jii.3 | chr17: 67517256- |
| | | | | | 67517949 |
| MAP2K7 | NA | 695.477 | 0.803436 | uc002mis.1 | chr19: 7970702- |
| | | | | | 7971931 |
| MAP3K12 | chr12: 53874276- | NA | NA | NA | NA |
| | 53874965 | | | | |
| MAP3K15 | chrX: 19378176- | NA | NA | NA | NA |
| | 19378866 | | | | |
| MAP3K5 | NA | 377.156 | −2.869232 | uc010kgw.1 | chr6: 136977131- |
| | | | | | 136977324 |
| MAP4K1 | NA | NA | NA | NA | NA |
| MAP6 | chr11: 75313905- | 201.717 | −3.424417 | uc001owv.3 | chr11: 75313905- |
| | 75316848 | | | | 75316848 |
| MAP7D1 | chr1: 36645860- | NA | NA | NA | NA |
| | 36646441 | | | | |
| MAP7D2 | NA | 314.667 | −5.735063 | uc004czq.2 | chrX: 20024831- |
| | | | | | 20026516 |
| MAP7D3 | NA | NA | NA | NA | NA |
| MAP9 | chr4: 156263812- | 1049.9 | −7.079601 | uc003ios.3 | chr4: 156263812- |
| | 156268934 | | | | 156268934 |
| MAPK14 | NA | 220.294 | −6.204244 | uc011dth.2 | chr6: 36063853- |
| | | | | | 36065831 |
| MAPK6 | NA | 727.572 | −9.047831 | uc002abp.3 | chr15: 52357198- |
| | | | | | 52358462 |
| MAPK | chr10: 49642939- | NA | NA | NA | NA |
| | 49643183 | | | | |
| MAPK8IP2 | NA | 208.365 | −4.210683 | uc003bmx.3 | chr22: 51049208- |
| | | | | | 51049979 |
| MAPK9 | chr5: 179660595- | 779.385 | −6.165719 | uc003mlt.4 | chr5: 179660595- |
| | 179663383 | | | | 179663514 |
| MARK1 | chr1: 220835509- | NA | NA | NA | NA |
| | 220837799 | | | | |
| MARK4 | NA | 476.955 | −5.830366 | uc002paz.2 | chr19: 45783833- |
| | | | | | 45783992 |
| MARS | NA | 1063.57 | −9.388647 | uc010srq.1 | chr12: 57909922- |
| | | | | | 57910064 |
| MARS2 | NA | 760.402 | −5.953375 | uc002uuq.3 | chr2: 198571912- |
| | | | | | 198573114 |
| MARVELD2 | chr5: 68737482- | NA | NA | NA | NA |
| | 68737890 | | | | |
| MAS1 | NA | 365.428 | −5.783976 | uc003qsz.3 | chr6: 160328966- |
| | | | | | 160329107 |
| MAST3 | chr19: 18260537- | NA | NA | NA | NA |
| | 18262499 | | | | |
| MAST4 | chr5: 66410077- | NA | NA | NA | NA |
| | 66410875 | | | | |
| MAT1A | chr10: 82031576- | NA | NA | NA | NA |
| | 82033536 | | | | |
| MAT2A | chr2: 85770896- | 592.685 | −4.038807 | uc010ysr.2 | chr2: 85770162- |
| | 85772403 | | | | 85770775 |
| MATN1 | chr1: 31184124- | NA | NA | NA | NA |
| | 31186428 | | | | |
| MATN2 | chr8: 99016041- | 265.391 | −10.753516 | uc003yib.1 | chr8: 99016041- |
| | 99016514 | | | | 99016514 |
| MATN3 | NA | 1224.56 | −12.562243 | uc002rdl.3 | chr2: 20191813- |
| | | | | | 20192871 |
| MATR3 | NA | 773.238 | −5.197222 | uc003ldt.3 | chr5: 138665085- |
| | | | | | 138667366 |
| MBD1 | NA | 551.169 | −5.349731 | uc002lef.3 | chr18: 47793252- |
| | | | | | 47793957 |
| MBD2 | chr18: 51729050- | 902.628 | −9.138454 | uc002lfg.2 | chr18: 51677971- |
| | 51729265 | | | | 51681681 |
| MBD5 | chr2: 149270511- | NA | NA | NA | NA |
| | 149271044 | | | | |
| MBIP | chr14: 36767764- | 553.704 | −5.568777 | uc001wtm.2 | chr14: 36767764- |
| | 36768288 | | | | 36768288 |
| MBL2 | NA | 634.209 | −6.039178 | uc001jjt.3 | chr10: 54525140- |
| | | | | | 54527896 |
| MBNL2 | NA | 213.229 | −5.656301 | uc010tij.2 | chr13: 98043704- |
| | | | | | 98046374 |
| MBNL3 | NA | NA | NA | NA | NA |
| MC1R | chr16: 89986621- | NA | NA | NA | NA |
| | 89987385 | | | | |
| MC5R | chr18: 13826743- | NA | NA | NA | NA |
| | 13826861 | | | | |
| MCC | NA | NA | NA | NA | NA |
| MCCC2 | NA | 298.394 | −6.926032 | uc003kbs.4 | chr5: 70952688- |
| | | | | | 70954530 |
| MCF2 | chrX: 138663930- | NA | NA | NA | NA |
| | 138664601 | | | | |
| MCFD2 | chr2: 47129009- | NA | NA | NA | NA |
| | 47132601 | | | | |
| MCHR2 | NA | NA | NA | NA | NA |
| MCM2 | NA | 1381.15 | −6.25191 | uc011bkn.2 | chr3: 127340617- |
| | | | | | 127341278 |
| MCM3AP | NA | 694.462 | −6.506625 | uc002zio.1 | chr21: 47655048- |
| | | | | | 47655181 |
| MCMBP | NA | 946.003 | −8.090572 | uc001let.1 | chr10: 121602294- |
| | | | | | 121602685 |
| MCMDC2 | NA | 436.65 | −6.406623 | uc003xwv.3 | chr8: 67796264- |
| | | | | | 67796854 |
| MCOLN3 | chr1: 85483765- | 201.168 | −4.493057 | uc001dko.3 | chr1: 85483765- |
| | 85484805 | | | | 85484805 |
| MCPH1 | NA | NA | NA | NA | NA |
| MCRS1 | NA | NA | NA | NA | NA |
| MCTP1 | chr5: 94042289- | NA | NA | NA | NA |
| | 94043200 | | | | |
| MCTS1 | chrX: 119746120- | 964.019 | −9.733943 | uc004esx.3 | chrX: 119746120- |
| | 119755016 | | | | 119755016 |
| MDC1 | chr6: 30667584- | NA | NA | NA | NA |
| | 30668241 | | | | |
| MDFIC | chr7: 114563552- | 509.281 | −7.57009 | uc022akf.1 | chr7: 114563552- |
| | 114563720 | | | | 114563720 |
| MDM1 | chr12: 68718785- | NA | NA | NA | NA |
| | 68720265 | | | | |
| MECOM | NA | 451.828 | −6.677247 | uc010hwk.1 | chr3: 168801287- |
| | | | | | 168802831 |
| MECP2 | NA | NA | NA | NA | NA |
| MED12 | NA | 637.419 | −7.732115 | uc004dyy.3 | chrX: 70362069- |
| | | | | | 70362304 |
| MED17 | NA | 706.611 | −1.889971 | uc001pel.2 | chr11: 93521355- |
| | | | | | 93522962 |
| MED19 | NA | 435.541 | −4.494564 | uc001nla.1 | chr11: 57471182- |
| | | | | | 57471509 |
| MED22 | NA | NA | NA | NA | NA |
| MED28 | NA | NA | NA | NA | NA |
| MED30 | chr8: 118552218- | NA | NA | NA | NA |
| | 118552501 | | | | |
| MED31 | NA | 926.686 | −10.09216 | uc002gdg.4 | chr17: 6546633- |
| | | | | | 6547786 |
| MED6 | chr14: 71050957- | 319.836 | −6.3261 | uc001xmf.3 | chr14: 71050957- |
| | 71051529 | | | | 71051529 |
| MED7 | NA | NA | NA | NA | NA |
| MEDAG | NA | 355.579 | −6.2151 | uc001uth.4 | chr13: 31498573- |
| | | | | | 31499709 |
| MEF2A | chr15: 100252977- | NA | NA | NA | NA |
| | 100256629 | | | | |
| MEF2C | NA | 1457.66 | −11.119391 | uc003kji.2 | chr5: 88014058- |
| | | | | | 88018420 |
| MEGF10 | NA | 1292.66 | −8.262511 | uc003kuh.4 | chr5: 126793011- |
| | | | | | 126796910 |
| MEGF9 | chr9: 123420280- | NA | NA | NA | NA |
| | 123421647 | | | | |
| MEIG1 | chr10: 15014641- | NA | NA | NA | NA |
| | 15014850 | | | | |
| MEIS3P1 | NA | 480.797 | −5.175553 | uc002gpc.3 | chr17: 15691010- |
| | | | | | 15693019 |
| MEP1A | NA | 815.72 | −6.873205 | uc010jzh.1 | chr6: 46806874- |
| | | | | | 46807519 |
| MERTK | chr2: 112786442- | NA | NA | NA | NA |
| | 112786945 | | | | |
| MEST | chr7: 130144898- | NA | NA | NA | NA |
| | 130146138 | | | | |
| METAP1 | chr4: 99982469- | 730.04 | −4.111178 | uc003huf.4 | chr4: 99982469- |
| | 99983960 | | | | 99983960 |
| METAP1D | NA | 733.051 | −6.20619 | uc002uhk.3 | chr2: 172945135- |
| | | | | | 172945587 |
| METAP2 | chr12: 95907681- | NA | NA | NA | NA |
| | 95909613 | | | | |
| METTL10 | chr10: 126447406- | 224.763 | −5.318861 | uc001lhy.1 | chr10: 126447406- |
| | 126449071 | | | | 126449071 |
| METTL12 | chr11: 62434524- | NA | NA | NA | NA |
| | 62434923 | | | | |
| METTL14 | NA | NA | NA | NA | NA |
| METTL15 | chr11: 28351943- | 696.467 | −6.071581 | uc001msh.2 | chr11: 28352389- |
| | 28355054 | | | | 28355054 |
| METTL16 | NA | 878.326 | −5.728369 | uc002fut.3 | chr17: 2319348- |
| | | | | | 2323263 |
| METTL21A | NA | NA | NA | NA | NA |
| METTL21B | chr12: 58174430- | NA | NA | NA | NA |
| | 58176324 | | | | |
| METTL2A | chr17: 60526091- | NA | NA | NA | NA |
| | 60527454 | | | | |
| METTL3 | chr14: 21969294- | NA | NA | NA | NA |
| | 21969850 | | | | |
| METTL4 | chr18: 2537524- | 557.203 | −9.868007 | uc010dkj.3 | chr18: 2537524- |
| | 2539135 | | | | 2539135 |
| METTL6 | chr3: 15455881- | NA | NA | NA | NA |
| | 15456315 | | | | |
| METTL8 | chr2: 172173913- | NA | NA | NA | NA |
| | 172180872 | | | | |
| METTL9 | NA | NA | NA | NA | NA |
| MEX3C | NA | 942.015 | −11.833307 | uc002lfc.4 | chr18: 48700920- |
| | | | | | 48702720 |
| MFAP1 | chr15: 44096733- | 358.477 | −3.559457 | uc001zth.1 | chr15: 44096733- |
| | 44097291 | | | | 44097291 |
| MFAP3L | NA | 451.258 | −6.246893 | uc003isn.4 | chr4: 170907748- |
| | | | | | 170912528 |
| MFHAS1 | chr8: 8641999- | NA | NA | NA | NA |
| | 8643531 | | | | |
| MFN1 | NA | 1160.38 | −7.941416 | uc003fjs.3 | chr3: 179109848- |
| | | | | | 179111008 |
| MFSD8 | chr4: 128838960- | 464.494 | −5.163575 | uc011cgv.1 | chr4: 128842679- |
| | 128841784 | | | | 128842913 |
| MGARP | chr4: 140187317- | NA | NA | NA | NA |
| | 140187752 | | | | |
| MGAT2 | chr14: 50089331- | 295.377 | −6.595879 | uc001wwr.3 | chr14: 50089331- |
| | 50090199 | | | | 50090199 |
| MGAT3 | NA | 338.01 | −4.368459 | uc003axv.4 | chr22: 39884955- |
| | | | | | 39888199 |
| MGAT4A | chr2: 99235569- | NA | NA | NA | NA |
| | 99242028 | | | | |
| MGAT5B | chr17: 74944921- | 642.989 | −5.127132 | uc002jtg.4 | chr17: 74878385- |
| | 74946471 | | | | 74878607 |
| MGME1 | chr20: 17970753- | NA | NA | NA | NA |
| | 17971762 | | | | |
| MGST1 | chr12: 16516976- | NA | NA | NA | NA |
| | 16517344 | | | | |
| MGST2 | chr4: 140625170- | 295.445 | −3.118996 | uc003ihy.3 | chr4: 140625303- |
| | 140625406 | | | | 140625406 |
| MIA2 | NA | 824.489 | −8.748487 | uc001wux.3 | chr14: 39722454- |
| | | | | | 39722575 |
| MICAL3 | chr22: 18317101- | NA | NA | NA | NA |
| | 18317215 | | | | |
| MICB | chr6_cox_hap2: | NA | NA | NA | NA |
| | 2987358-2988570 | | | | |
| MICU2 | chr13: 22066828- | 673.871 | −6.850497 | uc010tct.2 | chr13: 22066828- |
| | 22067387 | | | | 22067387 |
| MIEF1 | chr22: 39910329- | NA | NA | NA | NA |
| | 39914137 | | | | |
| MIEN1 | NA | 537.13 | −5.335124 | uc002hsq.3 | chr17: 37885409- |
| | | | | | 37885774 |
| MIER1 | chr1: 67452157- | NA | NA | NA | NA |
| | 67454302 | | | | |
| MINK1 | chr17: 4800583- | 611.115 | −3.244397 | uc010vsk.2 | chr17: 4800583- |
| | 4801356 | | | | 4801356 |
| MINPP1 | NA | NA | NA | NA | NA |
| MIOS | NA | 811.71 | −7.31556 | uc003srf.3 | chr7: 7646724- |
| | | | | | 7547110 |
| MIPEP | chr13: 24304328- | NA | NA | NA | NA |
| | 24304486 | | | | |
| MIS18A | NA | NA | NA | NA | NA |
| MIS18BP1 | chr14: 45672393- | NA | NA | NA | NA |
| | 45673311 | | | | |
| MITD1 | NA | 595.437 | −6.921452 | uc002szs.1 | chr2: 99785726- |
| | | | | | 99785837 |
| MITF | NA | NA | NA | NA | NA |
| MKI67 | NA | 1102.72 | −10.988088 | uc001lke.3 | chr10: 129894925- |
| | | | | | 129897453 |
| MKLN1 | NA | 524.957 | −4.840405 | uc011kpl.2 | chr7: 131172488- |
| | | | | | 131181398 |
| MKRN1 | chr7: 140155983- | 597.114 | −4.93754 | uc003vvu.4 | chr7: 140155983- |
| | 140156447 | | | | 140156447 |
| MKRN2OS | chr3: 12581280- | NA | NA | NA | NA |
| | 12581691 | | | | |
| MKS1 | NA | NA | NA | NA | NA |
| MKX | NA | NA | NA | NA | NA |
| MLEC | NA | 785.499 | −6.094766 | uc001tyy.1 | chr12: 121134349- |
| | | | | | 121139667 |
| MLF1 | NA | NA | NA | NA | NA |
| MLH1 | chr3: 37092145- | 1070.49 | −5.541789 | uc003cgl.3 | chr3: 37092145- |
| | 37092337 | | | | 37092337 |
| MLLT10 | NA | 1002.67 | −7.352891 | uc001iqs.3 | chr10: 22030868- |
| | | | | | 22032559 |
| MLLT11 | NA | NA | NA | NA | NA |
| MLLT3 | NA | NA | NA | NA | NA |
| MLLT4 | NA | 751.938 | −7.985108 | uc021zij.1 | chr6: 168370616- |
| | | | | | 168372700 |
| MMAA | NA | 1456.64 | −21.490569 | uc003ikh.4 | chr4: 146576587- |
| | | | | | 146581187 |
| MMACHC | NA | 988.571 | −6.465455 | uc009vxv.3 | chr1: 45974888- |
| | | | | | 45976739 |
| MMADHC | chr2: 150426147- | 2969.67 | −20.15297 | uc002txc.3 | chr2: 150426147- |
| | 150426487 | | | | 150426487 |
| MMD | chr17: 53469974- | NA | NA | NA | NA |
| | 53471694 | | | | |
| MME | NA | NA | NA | NA | NA |
| MMGT1 | NA | 586.624 | −8.10231 | uc022cet.1 | chrX: 135044231- |
| | | | | | 135047182 |
| MMP10 | NA | 1522.24 | −16.869343 | uc001phg.2 | chr11: 102641233- |
| | | | | | 102641523 |
| MMP15 | chr16: 58079351- | NA | NA | NA | NA |
| | 58080804 | | | | |
| MMP16 | chr8: 89049460- | NA | NA | NA | NA |
| | 89053688 | | | | |
| MMP7 | chr11: 102391239- | 1251.92 | −4.011546 | uc001phb.3 | chr11: 102391239- |
| | 102391506 | | | | 102391506 |
| MMP8 | NA | NA | NA | NA | NA |
| MMRN1 | NA | NA | NA | NA | NA |
| MMRN2 | chr10: 88695298- | NA | NA | NA | NA |
| | 88696499 | | | | |
| MMS22L | chr6: 97590037- | 284.804 | −7.008867 | uc003ppc.3 | chr6: 97701352- |
| | 97594681 | | | | 97702354 |
| MOB1A | NA | NA | NA | NA | NA |
| MOB1B | chr4: 71847775- | 567.188 | −8.175083 | uc003hfv.2 | chr4: 71841039- |
| | 71853891 | | | | 71842460 |
| MOB3C | NA | NA | NA | NA | NA |
| MOCS1 | NA | 446.707 | −7.726319 | uc003opb.3 | chr6: 39872034- |
| | | | | | 39874132 |
| MOCS2 | chr5: 52393895- | 1206.1 | −13.98207 | uc011cqf.2 | chr5: 52397927- |
| | 52394497 | | | | 52398054 |
| MON1A | NA | 975.933 | −4.621561 | uc003cyb.2 | chr3: 49946308- |
| | | | | | 49947547 |
| MON1B | chr16: 77232206- | NA | NA | NA | NA |
| | 77233543 | | | | |
| MORC2 | chr22: 31322600- | NA | NA | NA | NA |
| | 31322789 | | | | |
| MORC4 | chrX: 106057101- | NA | NA | NA | NA |
| | 106058137 | | | | |
| MORF4L2 | NA | 686.578 | −7.650829 | uc004ekw.3 | chrX: 102930426- |
| | | | | | 102931088 |
| MOV10L1 | NA | 1310.41 | −8.912094 | uc011arq.1 | chr22: 50581765- |
| | | | | | 50581881 |
| MPDU1 | NA | 920.662 | −4.90022 | uc002ghx.3 | chr17: 7490763- |
| | | | | | 7491527 |
| MPEG1 | NA | NA | NA | NA | NA |
| MPHOSPH8 | NA | 418.713 | −5.305648 | uc001umg.3 | chr13: 20245127- |
| | | | | | 20245258 |
| MPP7 | NA | NA | NA | NA | NA |
| MPPED2 | chr11: 30431618- | NA | NA | NA | NA |
| | 30433014 | | | | |
| MPRIP | chr17: 17088137- | NA | NA | NA | NA |
| | 17095962 | | | | |
| MR1 | chr1: 181024402- | NA | NA | NA | NA |
| | 181031074 | | | | |
| MRAP | chr21: 33686965- | NA | NA | NA | NA |
| | 33687094 | | | | |
| MRE11A | NA | 424.749 | −6.040094 | uc001peu.2 | chr11: 94150469- |
| | | | | | 94153290 |
| MRGPRX2 | NA | 413.015 | −5.230157 | uc001mph.3 | chr11: 19076003- |
| | | | | | 19076956 |
| MRM1 | NA | NA | NA | NA | NA |
| MROH7 | chr1: 55129467- | NA | NA | NA | NA |
| | 55129728 | | | | |
| MROH9 | chr1: 170967542- | NA | NA | NA | NA |
| | 170967946 | | | | |
| MRPL11 | chr11: 66202550- | NA | NA | NA | NA |
| | 66203583 | | | | |
| MRPL19 | NA | 371.363 | −5.658348 | uc002snl.3 | chr2: 75882412- |
| | | | | | 75889334 |
| MRPL2 | NA | 1547.44 | −4.53294 | uc003ots.1 | chr6: 43021767- |
| | | | | | 43022011 |
| MRPL22 | chr5: 154346458- | NA | NA | NA | NA |
| | 154348971 | | | | |
| MRPL35 | chr2: 86439550- | 1316.5 | −14.609574 | uc002srg.4 | chr2: 86437792- |
| | 86440477 | | | | 86440477 |
| MRPL36 | chr5: 1798499- | NA | NA | NA | NA |
| | 1798737 | | | | |
| MRPL42 | NA | NA | NA | NA | NA |
| MRPL46 | chr15: 89002708- | NA | NA | NA | NA |
| | 89007910 | | | | |
| MRPL48 | NA | 508.791 | −6.737641 | uc001ouh.4 | chr11: 73575416- |
| | | | | | 73575656 |
| MRPL50 | NA | NA | NA | NA | NA |
| MRPL57 | NA | 600.758 | −8.880167 | uc001unw.3 | chr13: 21751365- |
| | | | | | 21753220 |
| MRPL9 | chr1: 151732123- | 695.006 | −4.375258 | uc001eyv.3 | chr1: 151732123- |
| | 151732525 | | | | 151732525 |
| MRPS14 | NA | 462.369 | −4.507734 | uc001gkk.3 | chr1: 174982094- |
| | | | | | 174983804 |
| MRPS16 | NA | 732.409 | −6.408624 | uc001jts.1 | chr10: 75008601- |
| | | | | | 75010609 |
| MRPS18C | NA | 278.402 | −4.874063 | uc003hor.4 | chr4: 84382351- |
| | | | | | 84382929 |
| MRPS23 | NA | 578.264 | −5.438736 | uc002ivc.3 | chr17: 55916287- |
| | | | | | 55917143 |
| MRPS33 | chr7: 140705961- | NA | NA | NA | NA |
| | 140706229 | | | | |
| MRPS36 | chr5: 68525053- | NA | NA | NA | NA |
| | 68525985 | | | | |
| MRPS5 | NA | 207.398 | −8.427206 | uc002sub.3 | chr2: 95752952- |
| | | | | | 95753101 |
| MRPS6 | chr21: 35514901- | NA | NA | NA | NA |
| | 35515334 | | | | |
| MRS2 | chr6: 24423923- | NA | NA | NA | NA |
| | 24425816 | | | | |
| MS4A1 | NA | 538.935 | −5.196112 | uc009ymy.1 | chr11: 60231286- |
| | | | | | 60231785 |
| MS4A13 | chr11: 60310049- | NA | NA | NA | NA |
| | 60310191 | | | | |
| MS4A2 | NA | 228.979 | −5.847175 | uc001nop.3 | chr11: 59863130- |
| | | | | | 59865940 |
| MS4A4A | NA | NA | NA | NA | NA |
| MS4A6A | chr11: 59939080- | 639.411 | −3.042746 | uc001noq.3 | chr11: 59939080- |
| | 59939726 | | | | 59939726 |
| MSANTD3 | NA | NA | NA | NA | NA |
| MSH4 | chr1: 76378573- | NA | NA | NA | NA |
| | 76378923 | | | | |
| MSL | NA | 571.452 | −7.620023 | uc002hua.4 | chr17: 38290623- |
| | | | | | 38293045 |
| MSL2 | chr3: 135867760- | NA | NA | NA | NA |
| | 135869988 | | | | |
| MSL3 | NA | 235.083 | −6.444387 | uc004cuw.3 | chrX: 11793199- |
| | | | | | 11793872 |
| MSRA | NA | NA | NA | NA | NA |
| MSX1 | chr4: 4864871- | NA | NA | NA | NA |
| | 4865660 | | | | |
| MT1A | NA | NA | NA | NA | NA |
| MT1E | chr16: 56660884- | NA | NA | NA | NA |
| | 56661024 | | | | |
| MT1F | NA | NA | NA | NA | NA |
| MT1G | chr16: 56700653- | NA | NA | NA | NA |
| | 56700790 | | | | |
| MT1H | chr16: 56704902- | NA | NA | NA | NA |
| | 56705041 | | | | |
| MT1M | chr16: 56667755- | 568.486 | −9.106859 | uc002ejn.3 | chr16: 56667755- |
| | 56667898 | | | | 56667898 |
| MT1X | chr16: 56717962- | NA | NA | NA | NA |
| | 56718108 | | | | |
| MT2A | chr16: 56643267- | NA | NA | NA | NA |
| | 56643409 | | | | |
| MTCP1 | chrX: 154292309- | NA | NA | NA | NA |
| | 154293674 | | | | |
| MTDH | chr8: 98736899- | 361.118 | −6.529536 | uc003yhz.3 | chr8: 98736899- |
| | 98742488 | | | | 98742488 |
| MTERF2 | NA | NA | NA | NA | NA |
| MTF2 | chr1: 93602585- | NA | NA | NA | NA |
| | 93604638 | | | | |
| MTFMT | NA | 1098.24 | −10.090352 | uc002aof.4 | chr15: 65293850- |
| | | | | | 65295399 |
| MTFR1 | chr8: 66620361- | NA | NA | NA | NA |
| | 66620573 | | | | |
| MTG2 | chr20: 60776134- | NA | NA | NA | NA |
| | 60777810 | | | | |
| MTHFD2 | NA | NA | NA | NA | NA |
| MTHFD2L | chr4: 75107325- | 508.043 | −5.918091 | uc011cbj.2 | chr4: 75107325- |
| | 75107916 | | | | 75107916 |
| MTHFR | NA | NA | NA | NA | NA |
| MTHFS | NA | 1013.31 | −6.474058 | uc021srp.1 | chr15: 80135889- |
| | | | | | 80137551 |
| MTMR1 | NA | 878.331 | −7.728709 | uc011mya.1 | chrX: 149931203- |
| | | | | | 149933397 |
| MTMR11 | chr1: 149900543- | 1249.52 | −8.667684 | uc010pbm.1 | chr1: 149902257- |
| | 149901020 | | | | 149902831 |
| MTMR14 | NA | 474.795 | −5.131939 | uc003brz.3 | chr3: 9743658- |
| | | | | | 9744078 |
| MTMR6 | NA | NA | NA | NA | NA |
| MTMR8 | NA | NA | NA | NA | NA |
| MTMR9 | chr8: 11180298- | NA | NA | NA | NA |
| | 11185654 | | | | |
| MTOR | NA | 315.005 | −6.175893 | uc001asc.3 | chr1: 11166588- |
| | | | | | 11167541 |
| MTR | chr1: 237060945- | 692.982 | −6.250084 | uc001hyi.4 | chr1: 237060945- |
| | 237067281 | | | | 237067281 |
| MTRF1L | NA | NA | NA | NA | NA |
| MTRNR2L10 | NA | NA | NA | NA | NA |
| MTX2 | NA | NA | NA | NA | NA |
| MUC15 | chr11: 26580579- | NA | NA | NA | NA |
| | 26582611 | | | | |
| MUC2 | NA | 362.804 | −5.933495 | uc001lsx.1 | chr11: 1104264- |
| | | | | | 1104417 |
| MUCL1 | NA | NA | NA | NA | NA |
| MUL1 | NA | 559.669 | −6.261952 | uc001bdi.4 | chr1: 20825941- |
| | | | | | 20827182 |
| MURC | NA | NA | NA | NA | NA |
| MUS81 | NA | NA | NA | NA | NA |
| MUTYH | chr1: 45794914- | NA | NA | NA | NA |
| | 45794977 | | | | |
| MVK | NA | NA | NA | NA | NA |
| MXRA7 | chr17: 74671809- | 346.21 | −7.585167 | uc002jsl.3 | chr17: 74675651- |
| | 74673669 | | | | 74676961 |
| MYB | NA | 651.201 | −7.516773 | uc003qfc.3 | chr6: 135539119- |
| | | | | | 135540311 |
| MYBBP1A | chr17: 4442191- | NA | NA | NA | NA |
| | 4442709 | | | | |
| MYCT1 | NA | 1279.71 | −9.509562 | uc003qpc.4 | chr6: 153043389- |
| | | | | | 153045715 |
| MYD88 | NA | 244.154 | −6.497243 | uc011ayj.2 | chr3: 38182644- |
| | | | | | 38184512 |
| MYEF2 | NA | 451.598 | −11.296831 | uc001zwg.4 | chr15: 48431629- |
| | | | | | 48435104 |
| MYF5 | NA | NA | NA | NA | NA |
| MYH3 | NA | NA | NA | NA | NA |
| MYL1 | NA | 239.239 | −5.671791 | uc002veb.3 | chr2: 211154868- |
| | | | | | 211155191 |
| MYL12A | chr18: 3255917- | NA | NA | NA | NA |
| | 3256234 | | | | |
| MYLIP | chr6: 16146983- | NA | NA | NA | NA |
| | 16148478 | | | | |
| MYLK3 | chr16: 46736194- | NA | NA | NA | NA |
| | 46741615 | | | | |
| MYLK4 | NA | 674.502 | −5.441523 | uc003mtx.4 | chr6: 2663863- |
| | | | | | 2668133 |
| MYO1B | chr2: 192288687- | 1505.88 | −12.488781 | uc002usq.2 | chr2: 192288687- |
| | 192290115 | | | | 192290115 |
| MYO3B | chr2: 171238638- | NA | NA | NA | NA |
| | 171239276 | | | | |
| MYO9A | NA | 431.804 | −9.607694 | uc002atn.1 | chr15: 72181813- |
| | | | | | 72185263 |
| MYOM2 | chr8: 2092906- | NA | NA | NA | NA |
| | 2093380 | | | | |
| MYOT | chr5: 137223075- | NA | NA | NA | NA |
| | 137223540 | | | | |
| MYOZ2 | NA | 477.157 | −7.359383 | uc003icp.4 | chr4: 120107356- |
| | | | | | 120108944 |
| MYZAP | NA | NA | NA | NA | NA |
| N6AMT2 | NA | 1319.45 | −11.70952 | uc001uno.1 | chr13: 21303073- |
| | | | | | 21303218 |
| NBEAP1 | NA | 428.368 | −6.53265 | uc010tze.1 | chr15: 20874797- |
| | | | | | 20874834 |
| NBN | chr8: 90945564- | 695.686 | −7.547016 | uc003yei.1 | chr8: 90945564- |
| | 90947823 | | | | 90947809 |
| NCAM2 | NA | NA | NA | NA | NA |
| NCAPG2 | NA | NA | NA | NA | NA |
| NCBP1 | NA | NA | NA | NA | NA |
| NCF1 | chr7: 74197565- | 740.643 | −5.962718 | uc011kfh.1 | chr7: 74197565- |
| | 74197975 | | | | 74197975 |
| NCF2 | NA | 423.455 | −4.978247 | uc001gqj.4 | chr1: 183524697- |
| | | | | | 183525252 |
| NCOA7 | chr6: 126249918- | NA | NA | NA | NA |
| | 126253176 | | | | |
| NDE1 | chr16: 15818109- | NA | NA | NA | NA |
| | 15820208 | | | | |
| NDN | NA | NA | NA | NA | NA |
| NDUFA1 | NA | NA | NA | NA | NA |
| NDUFA13 | NA | NA | NA | NA | NA |
| NDUFA9 | NA | 396.903 | −5.421258 | uc001qnc.3 | chr12: 4796275- |
| | | | | | 4796720 |
| NDUFAF2 | NA | 488.689 | −6.814918 | NA | NA |
| NDUFAF4 | chr6: 97337187- | 1070.68 | −8.859478 | uc003pow.3 | chr6: 97337187- |
| | 97338979 | | | | 97338979 |
| NDUFAF5 | chr20: 13797857- | NA | NA | NA | NA |
| | 13799067 | | | | |
| NDUFAF6 | NA | NA | NA | NA | NA |
| NDUFB10 | chr16: 2011908- | NA | NA | NA | NA |
| | 2011976 | | | | |
| NDUFB2 | chr7: 140406363- | NA | NA | NA | NA |
| | 140406446 | | | | |
| NDUFB4 | NA | 797.712 | −4.006915 | uc003edt.3 | chr3: 120320141- |
| | | | | | 120321258 |
| NDUFB5 | chr3: 179341829- | NA | NA | NA | NA |
| | 179342288 | | | | |
| NDUFC1 | NA | 493.168 | −9.457095 | uc003ihs.3 | chr4: 140211071- |
| | | | | | 140211246 |
| NDUFS2 | NA | 1625.79 | −3.46401 | uc001fyv.3 | chr1: 161183984- |
| | | | | | 161184184 |
| NDUFS3 | chr11: 47606034- | NA | NA | NA | NA |
| | 47606115 | | | | |
| NDUFS6 | NA | NA | NA | NA | NA |
| NEB | chr2: 152341853- | 466.06 | −8.697627 | uc010zbz.2 | chr2: 152341853- |
| | 152342273 | | | | 152342273 |
| NEBL | NA | 263.593 | −4.321902 | uc001iqi.3 | chr10: 21068903- |
| | | | | | 21074675 |
| NECAB1 | NA | NA | NA | NA | NA |
| NEDD4 | chr15: 56119122- | NA | NA | NA | NA |
| | 56122094 | | | | |
| NEDD4L | NA | NA | NA | NA | NA |
| NEDD9 | chr6: 11183531- | NA | NA | NA | NA |
| | 11185394 | | | | |
| NEFH | chr22: 29886693- | 262.108 | −4.161698 | uc003afo.3 | chr22: 29886693- |
| | 29887277 | | | | 29887277 |
| NEIL2 | chr8: 11643783- | NA | NA | NA | NA |
| | 11644854 | | | | |
| NEIL3 | NA | 600.788 | −4.653912 | uc003iut.2 | chr4: 178283626- |
| | | | | | 178284092 |
| NEK1 | chr4: 170475194- | 1263.92 | −11.334548 | uc003isb.2 | chr4: 170314421- |
| | 170476848 | | | | 170315660 |
| NEK10 | chr3: 27257097- | NA | NA | NA | NA |
| | 27257322 | | | | |
| NEK5 | chr13: 52638900- | NA | NA | NA | NA |
| | 52639542 | | | | |
| NEK8 | NA | NA | NA | NA | NA |
| NES | NA | 344.188 | −4.519712 | uc001fpq.3 | chr1: 156638556- |
| | | | | | 156639113 |
| NEURL1 | NA | NA | NA | NA | NA |
| NEUROD4 | chr12: 55421220- | NA | NA | NA | NA |
| | 55423801 | | | | |
| NEXN | chr1: 78408515- | 1105.83 | −7.985332 | uc001dib.4 | chr1: 78408515- |
| | 78409578 | | | | 78409578 |
| NF1 | chr17: 29546152- | NA | NA | NA | NA |
| | 29546377 | | | | |
| NFASC | chr1: 204985668- | NA | NA | NA | NA |
| | 204991950 | | | | |
| NFAT5 | chr16: 69718885- | 1366.25 | −8.520657 | uc002exi.3 | chr16: 69729275- |
| | 69720072 | | | | 69729591 |
| NFATC4 | chr14: 24846150- | NA | NA | NA | NA |
| | 24848810 | | | | |
| NFE2L2 | chr2: 178098205- | NA | NA | NA | NA |
| | 178098690 | | | | |
| NFKBIA | NA | 228.901 | −4.907543 | uc001wtf.4 | chr14: 35870716- |
| | | | | | 35871218 |
| NFU1 | chr2: 69623245- | NA | NA | NA | NA |
| | 69623377 | | | | |
| NFYA | NA | 680.081 | −7.303833 | uc003opo.3 | chr6: 41065150- |
| | | | | | 41070146 |
| NGB | chr14: 77731834- | NA | NA | NA | NA |
| | 77732878 | | | | |
| NGBN | chr14: 23947236- | 830.782 | −12.522897 | uc001wjy.3 | chr14: 23947236- |
| | 23947402 | | | | 23947402 |
| NGEF | chr2: 233743396- | NA | NA | NA | NA |
| | 233744198 | | | | |
| NGFRAP1 | NA | 716.878 | −7.061391 | uc004ekh.3 | chrX: 102632756- |
| | | | | | 102633001 |
| NGLY1 | chr3: 25760435- | NA | NA | NA | NA |
| | 25760950 | | | | |
| NGRN | chr15: 90815021- | NA | NA | NA | NA |
| | 90815443 | | | | |
| NHP2L1 | chr22: 42069937- | NA | NA | NA | NA |
| | 42070936 | | | | |
| NHSL1 | chr6: 138743181- | 914.206 | −6.888536 | uc003qhy.2 | chr6: 138767927- |
| | 138745217 | | | | 138768123 |
| NID1 | chr1: 236139132- | NA | NA | NA | NA |
| | 236141166 | | | | |
| NID2 | NA | 482.419 | −5.137794 | uc001wzp.3 | chr14: 52479427- |
| | | | | | 52480887 |
| NIF3L1 | NA | 232.813 | −6.652392 | uc002uwq.2 | chr2: 201768265- |
| | | | | | 201768655 |
| NIFK | chr2: 122484521- | NA | NA | NA | NA |
| | 122485299 | | | | |
| NIN | NA | 386.566 | −7.45279 | uc001wyi.3 | chr14: 51186481- |
| | | | | | 51190180 |
| NINL | chr20: 25433338- | 342.355 | −3.797401 | uc002wuw.1 | chr20: 25433338- |
| | 25434086 | | | | 25434086 |
| NIPAL2 | NA | NA | NA | NA | NA |
| NIT1 | chr1: 161093946- | NA | NA | NA | NA |
| | 161095235 | | | | |
| NKAIN2 | chr6: 124979626- | NA | NA | NA | NA |
| | 124980104 | | | | |
| NKAIN4 | NA | NA | NA | NA | NA |
| NKAP | chrX: 119059013- | NA | NA | NA | NA |
| | 119059182 | | | | |
| NKRF | NA | NA | NA | NA | NA |
| NKTR | NA | 455.571 | −10.632795 | uc003clo.3 | chr3: 42687468- |
| | | | | | 42690233 |
| NLN | NA | NA | NA | NA | NA |
| NLRP2 | NA | NA | NA | NA | NA |
| NLRP8 | NA | 784.943 | −7.9182 | uc002qmh.3 | chr19: 56499280- |
| | | | | | 56499995 |
| NMB | NA | 575.047 | −8.311893 | uc002bkz.3 | chr15: 85198360- |
| | | | | | 85198604 |
| NME5 | NA | 540.112 | −7.264984 | uc003lce.3 | chr5: 137450861- |
| | | | | | 137451369 |
| NME7 | NA | NA | NA | NA | NA |
| NME9 | NA | 334.142 | −5.422168 | uc003esg.3 | chr3: 138020054- |
| | | | | | 138020481 |
| NMI | chr2: 152133769- | 458.955 | −7.073818 | uc010zbx.1 | chr2: 152132068- |
| | 152135288 | | | | 152132259 |
| NMRK2 | NA | 476.149 | −4.926586 | uc002lyz.4 | chr19: 3942272- |
| | | | | | 3942414 |
| NMT1 | NA | NA | NA | NA | NA |
| NMUR2 | NA | NA | NA | NA | NA |
| NOA1 | NA | NA | NA | NA | NA |
| NOC3L | chr10: 96092989- | NA | NA | NA | NA |
| | 96093933 | | | | |
| NOD1 | NA | NA | NA | NA | NA |
| NOD2 | chr16: 50765731- | NA | NA | NA | NA |
| | 50766987 | | | | |
| NOL4 | NA | NA | NA | NA | NA |
| NOL8 | chr9: 95059640- | NA | NA | NA | NA |
| | 95060117 | | | | |
| NOP14 | chr4: 2939664- | NA | NA | NA | NA |
| | 2939915 | | | | |
| NOS3 | chr7: 150711258- | NA | NA | NA | NA |
| | 150711687 | | | | |
| NOSTRIN | NA | 648.667 | −7.940967 | uc002uef.3 | chr2: 169721481- |
| | | | | | 169721849 |
| NOTCH1 | NA | 576.953 | −6.26778 | uc004chz.3 | chr9: 139388896- |
| | | | | | 139390522 |
| NOTCH2 | chr1: 120454176- | NA | NA | NA | NA |
| | 120457928 | | | | |
| NOTCH3 | NA | 565.96 | −5.137274 | uc002nan.3 | chr19: 15270444- |
| | | | | | 15271472 |
| NOX1 | NA | 426.554 | −4.848856 | uc004egj.3 | chrX: 100098313- |
| | | | | | 100098940 |
| NOX5 | NA | NA | NA | NA | NA |
| NOXRED1 | chr14: 77860365- | 833.484 | −15.632915 | uc001xtr.3 | chr14: 77860365- |
| | 77860973 | | | | 77860973 |
| NPAS3 | NA | 694.718 | −6.724189 | uc001wrs.3 | chr14: 34270316- |
| | | | | | 34273382 |
| NPC1L1 | NA | NA | NA | NA | NA |
| NPEPPS | NA | 450.175 | −4.530498 | uc010wkt.2 | chr17: 45699287- |
| | | | | | 45700642 |
| NPFFR2 | NA | 1209.16 | −13.810218 | uc003hgg.2 | chr4: 73013530- |
| | | | | | 73013918 |
| NPHS1 | NA | NA | NA | NA | NA |
| NPL | NA | 397.946 | −11.015261 | uc010pnx.2 | chr1: 182791259- |
| | | | | | 182791334 |
| NPM1 | NA | NA | NA | NA | NA |
| NPPB | chr1: 11917521- | NA | NA | NA | NA |
| | 11917711 | | | | |
| NPR3 | NA | 569.323 | −6.054896 | uc003jhw.2 | chr5: 32786452- |
| | | | | | 32791830 |
| NQO1 | NA | 1051.2 | −6.904812 | uc002exp.3 | chr16: 69743304- |
| | | | | | 69744878 |
| NR0B2 | chr1: 27237975- | NA | NA | NA | NA |
| | 27238335 | | | | |
| NR1D2 | NA | NA | NA | NA | NA |
| NR1H3 | chr11: 47290248- | NA | NA | NA | NA |
| | 47290584 | | | | |
| NR1H4 | NA | 506.224 | −6.571092 | uc001thp.2 | chr12: 100957268- |
| | | | | | 100957645 |
| NR1I3 | chr1: 161199456- | NA | NA | NA | NA |
| | 161199586 | | | | |
| NR2C2 | chr3: 15084516- | NA | NA | NA | NA |
| | 15090780 | | | | |
| NR2C2AP | NA | 541.618 | −5.742031 | uc002nlx.3 | chr19: 19312224- |
| | | | | | 19312733 |
| NR2F2 | NA | NA | NA | NA | NA |
| NR5A2 | NA | NA | NA | NA | NA |
| NRCAM | NA | NA | NA | NA | NA |
| NRG1 | chr8: 32600287- | 519.992 | −1.736759 | uc003xip.3 | chr8: 32600287- |
| | 32600770 | | | | 32600770 |
| NRG2 | NA | 340.944 | −6.15753 | uc003lev.2 | chr5: 139226364- |
| | | | | | 139227501 |
| NRG3 | chr10: 84745362- | NA | NA | NA | NA |
| | 84746935 | | | | |
| NRG4 | NA | 532.386 | −7.65789 | uc002bbo.3 | chr15: 76234328- |
| | | | | | 76235978 |
| NRIP1 | NA | NA | NA | NA | NA |
| NRK | NA | NA | NA | NA | NA |
| NRP1 | NA | NA | NA | NA | NA |
| NRP2 | NA | 755.525 | −6.776111 | uc002vat.3 | chr2: 206608304- |
| | | | | | 206608422 |
| NRTN | chr19: 5828185- | NA | NA | NA | NA |
| | 5828335 | | | | |
| NSD1 | chr5: 176563092- | NA | NA | NA | NA |
| | 176563332 | | | | |
| NSG1 | NA | NA | NA | NA | NA |
| NSL1 | chr1: 212899495- | 595.525 | −4.944611 | uc001hjm.3 | chr1: 212899495- |
| | 212912028 | | | | 212912028 |
| NSMCE4A | chr10: 123716603- | 1207.61 | −9.461411 | uc001lfs.3 | chr10: 123716603- |
| | 123716774 | | | | 123716774 |
| NSUN2 | NA | 470.129 | −5.92551 | uc003jdt.3 | chr5: 6599352- |
| | | | | | 6600038 |
| NSUN3 | NA | NA | NA | NA | NA |
| NSUN5P2 | NA | 604.447 | −7.258824 | uc003twm.3 | chr7: 72418832- |
| | | | | | 72418922 |
| NSUN6 | NA | NA | NA | NA | NA |
| NSUN7 | chr4: 40776977- | 552.83 | −8.56521 | uc003gvh.2 | chr4: 40776977- |
| | 40777473 | | | | 40777473 |
| NT5C1B | NA | 643.165 | −12.859234 | uc010yju.2 | chr2: 18744137- |
| | | | | | 18745061 |
| NTF3 | NA | 760.324 | −8.584455 | uc001qnk.4 | chr12: 5604155- |
| | | | | | 5604465 |
| NTMT1 | NA | 446.503 | −5.525523 | uc011mbs.1 | chr9: 132397595- |
| | | | | | 132397879 |
| NTRK2 | NA | 493.927 | −6.036994 | uc004anv.1 | chr9: 87425495- |
| | | | | | 87430621 |
| NTRK3 | NA | 361.676 | −4.84583 | uc002bmh.2 | chr15: 88419988- |
| | | | | | 88420165 |
| NUAK1 | NA | 718.362 | −5.354271 | uc001tlj.1 | chr12: 106457125- |
| | | | | | 106460579 |
| NUB1 | NA | NA | NA | NA | NA |
| NUBPL | chr14: 32328387- | NA | NA | NA | NA |
| | 32330429 | | | | |
| NUCKS1 | chr1: 205681947- | NA | NA | NA | NA |
| | 205687407 | | | | |
| NUDCD1 | chr8: 110253148- | NA | NA | NA | NA |
| | 110255237 | | | | |
| NUDCD2 | chr5: 162880586- | NA | NA | NA | NA |
| | 162880972 | | | | |
| NUDCD3 | NA | 494.612 | −5.244314 | uc003tkz.3 | chr7: 44421965- |
| | | | | | 44425609 |
| NUDT10 | NA | 439.22 | −7.899343 | uc004dph.3 | chrX: 51079092- |
| | | | | | 51080377 |
| NUDT13 | NA | NA | NA | NA | NA |
| NUDT2 | NA | NA | NA | NA | NA |
| NUDT21 | chr16: 56463048- | NA | NA | NA | NA |
| | 56466623 | | | | |
| NUDT5 | NA | 605.879 | −5.381491 | uc001ilj.3 | chr10: 12209573- |
| | | | | | 12209700 |
| NUDT7 | chr16: 77759512- | 987.511 | −14.649194 | uc002fff.3 | chr16: 77759512- |
| | 77760697 | | | | 77760697 |
| NUFIP2 | chr17: 27582854- | NA | NA | NA | NA |
| | 27591556 | | | | |
| NUMA1 | NA | 783.849 | −6.070138 | uc009ysy.2 | chr11: 71728045- |
| | | | | | 71728597 |
| NUMB | chr14: 73741918- | NA | NA | NA | NA |
| | 73743285 | | | | |
| NUP107 | chr12: 69136243- | NA | NA | NA | NA |
| | 69136473 | | | | |
| NUP133 | NA | NA | NA | NA | NA |
| NUP160 | chr11: 47799670- | 1665.24 | −11.304681 | uc021qiu.1 | chr11: 47799670- |
| | 47800656 | | | | 47800562 |
| NUP188 | NA | 477.089 | −5.816282 | uc004bwu.3 | chr9: 131765896- |
| | | | | | 131766259 |
| NUP205 | NA | NA | NA | NA | NA |
| NUP210P1 | NA | NA | NA | NA | NA |
| NUP214 | NA | 352.165 | −6.258106 | uc010mzf.3 | chr9: 134070759- |
| | | | | | 134071045 |
| NUP35 | NA | 247.811 | −6.172364 | uc010zfs.2 | chr2: 184025860- |
| | | | | | 184026408 |
| NUP37 | NA | 734.139 | −2.359867 | uc001tjc.3 | chr12: 102467973- |
| | | | | | 102468131 |
| NUP62CL | NA | 473.835 | −5.733315 | uc004ena.3 | chrX: 106366657- |
| | | | | | 106367562 |
| NUP85 | NA | 1035.28 | −9.02277 | uc002jng.1 | chr17: 73231775- |
| | | | | | 73231854 |
| NUP88 | chr17: 5291837- | 619.936 | −9.973897 | uc010vsy.2 | chr17: 5291837- |
| | 5292090 | | | | 5292090 |
| NUP93 | NA | NA | NA | NA | NA |
| NUPL1 | chr13: 25905720- | NA | NA | NA | NA |
| | 25910478 | | | | |
| NUSAP1 | chr15: 41672385- | NA | NA | NA | NA |
| | 41673248 | | | | |
| NVL | chr1: 224415036- | NA | NA | NA | NA |
| | 224415327 | | | | |
| NXF5 | NA | 959.488 | −6.316019 | uc011mrk.1 | chrX: 101087085- |
| | | | | | 101087239 |
| NXT2 | NA | 408.487 | −6.909668 | uc004eoe.2 | chrX: 108785918- |
| | | | | | 108787927 |
| OARD1 | chr6: 41034531- | NA | NA | NA | NA |
| | 41035073 | | | | |
| OAS1 | NA | 328.76 | −6.255066 | uc010syo.2 | chr12: 113354319- |
| | | | | | 113354760 |
| OAS3 | chr12: 113379732- | NA | NA | NA | NA |
| | 113380100 | | | | |
| OAT | chr10: 126085872- | NA | NA | NA | NA |
| | 126086510 | | | | |
| OBFC1 | chr10: 105637318- | NA | NA | NA | NA |
| | 105642441 | | | | |
| OC90 | NA | 676.109 | −7.533595 | uc003ytg.2 | chr8: 133036467- |
| | | | | | 133036727 |
| OCEL1 | NA | 1164.14 | −5.231913 | uc002nfp.3 | chr19: 17339735- |
| | | | | | 17340028 |
| ODAM | NA | 1007.34 | −7.78842 | uc003hfc.3 | chr4: 71069892- |
| | | | | | 71070293 |
| OGFR | NA | 884.562 | −7.795273 | uc002ydj.3 | chr20: 61445002- |
| | | | | | 61445352 |
| OGN | NA | NA | NA | NA | NA |
| OGT | NA | 694.231 | −5.756156 | uc004eaa.2 | chrX: 70793645- |
| | | | | | 70795747 |
| OLFM4 | NA | 858.78 | −8.959844 | uc001vhl.3 | chr13: 53624907- |
| | | | | | 53626196 |
| OLFML1 | NA | NA | NA | NA | NA |
| OLFML2B | NA | NA | NA | NA | NA |
| OLFML3 | chr1: 114524392- | NA | NA | NA | NA |
| | 114524875 | | | | |
| OOEP | NA | 269.532 | −3.513168 | uc003pgu.4 | chr6: 74078280- |
| | | | | | 74078506 |
| OOSP2 | chr11: 59814547- | NA | NA | NA | NA |
| | 59815516 | | | | |
| OPN4 | chr10: 88423749- | NA | NA | NA | NA |
| | 88423870 | | | | |
| OPRK1 | NA | 575.588 | −5.242228 | uc022aup.1 | chr8: 54138276- |
| | | | | | 54141856 |
| OPRM1 | chr6: 154439857- | NA | NA | NA | NA |
| | 154440594 | | | | |
| OR11A1 | NA | 248.177 | −4.603861 | uc003nmg.3 | chr6: 29393281- |
| | | | | | 29394470 |
| OR12D3 | chr6: 29341200- | NA | NA | NA | NA |
| | 29342113 | | | | |
| OR13A1 | chr10: 45798102- | NA | NA | NA | NA |
| | 45798883 | | | | |
| OR2A9P | NA | 879.573 | −5.947149 | uc003wei.2 | chr7: 143997399- |
| | | | | | 143997598 |
| OR2B3 | NA | NA | NA | NA | NA |
| OR2H1 | NA | NA | NA | NA | NA |
| OR2J3 | NA | 464.941 | −4.967909 | uc011hhl.2 | chr6_mcf_hap5: |
| | | | | | 444969-445896 |
| OR4C12 | NA | NA | NA | NA | NA |
| OR51B5 | NA | NA | NA | NA | NA |
| OR51D1 | NA | 562.699 | −5.212676 | uc010qyk.2 | chr11: 4661996- |
| | | | | | 4662068 |
| OR51E2 | NA | 1894.87 | −16.090418 | uc001lzk.2 | chr11: 4701401- |
| | | | | | 4702978 |
| OR52K1 | chr11: 4511076- | NA | NA | NA | NA |
| | 4511138 | | | | |
| OR7E12P | chr11: 3411937- | NA | NA | NA | NA |
| | 3412607 | | | | |
| OR9Q1 | chr11: 57947850- | NA | NA | NA | NA |
| | 57949038 | | | | |
| ORC1 | chr1: 52838501- | NA | NA | NA | NA |
| | 52838852 | | | | |
| ORC4 | NA | 395.832 | −6.10338 | uc002twj.3 | chr2: 148687966- |
| | | | | | 148693078 |
| ORC6 | chr16: 46727099- | NA | NA | NA | NA |
| | 46728576 | | | | |
| ORMDL1 | NA | NA | NA | NA | NA |
| ORMDL2 | NA | 717.502 | −7.781309 | uc001shw.1 | chr12: 56214180- |
| | | | | | 56214959 |
| OSBP2 | NA | 323.145 | −5.2379 | uc011ala.1 | chr22: 31302327- |
| | | | | | 31303811 |
| OSBPL10 | NA | 578.758 | −6.291906 | uc003ceu.1 | chr3: 31699403- |
| | | | | | 31705510 |
| OSBPL1A | chr18: 21742011- | 377.46 | −8.772797 | uc002kvf.3 | chr18: 21771629- |
| | 21743142 | | | | 21776079 |
| OSBPL2 | NA | 1075.81 | −12.015268 | uc002yck.1 | chr20: 60868944- |
| | | | | | 60871269 |
| OSBPL35 | chr7: 24836164- | NA | NA | NA | NA |
| | 24839801 | | | | |
| OSGEPL1 | chr2: 190611386- | 769.968 | −6.91387 | uc002uqz.1 | chr2: 190611386- |
| | 190611894 | | | | 190611894 |
| OSMR | NA | NA | NA | NA | NA |
| OTOF | NA | NA | NA | NA | NA |
| OTOGL | NA | 1242.23 | −8.982257 | uc001szd.3 | chr12: 80771829- |
| | | | | | 80772870 |
| OTOR | chr20: 16731764- | NA | NA | NA | NA |
| | 16732809 | | | | |
| OTUD1 | NA | NA | NA | NA | NA |
| OTUD6B | NA | 1661.75 | −10.072529 | uc003yeu.4 | chr8: 92097097- |
| | | | | | 92099323 |
| OTUD7A | NA | 469.594 | −5.562474 | uc010baa.1 | chr15: 31850386- |
| | | | | | 31851109 |
| OTX1 | chr2: 63283452- | NA | NA | NA | NA |
| | 63284966 | | | | |
| OVOL2 | chr20: 18004796- | 291.288 | −7.651077 | uc002wqi.1 | chr20: 18004796- |
| | 18005279 | | | | 18005279 |
| OXA1L | NA | 563.891 | −6.739103 | uc001wgn.2 | chr14: 23240768- |
| | | | | | 23240998 |
| OXCT1 | chr5: 41730167- | NA | NA | NA | NA |
| | 41731830 | | | | |
| OXGR1 | NA | 904.151 | −7.734293 | uc001vmx.1 | chr13: 97637973- |
| | | | | | 97638999 |
| OXSR1 | chr3: 38271952- | 596.474 | −6.079549 | uc010hha.1 | chr3: 38271952- |
| | 38272184 | | | | 38272184 |
| P2RY1 | chr3: 152554694- | NA | NA | NA | NA |
| | 152555843 | | | | |
| P2RY10 | NA | 744.835 | −6.075441 | uc004ede.3 | chrX: 78217038- |
| | | | | | 78217438 |
| P2RY12 | NA | NA | NA | NA | NA |
| P4HA1 | chr10: 74766980- | 932.055 | −7.483303 | uc021ptj.1 | chr10: 74766980- |
| | 74767979 | | | | 74767979 |
| P4HA2 | chr5: 131528304- | NA | NA | NA | NA |
| | 131528702 | | | | |
| PABPC1L | NA | 813.556 | −2.230357 | uc010ggv.1 | chr20: 43567767- |
| | | | | | 43567962 |
| PABPC4L | NA | 271.865 | −6.127914 | uc010ioe.3 | chr4: 135117489- |
| | | | | | 135121061 |
| PABPC5 | chrX: 90691726- | NA | NA | NA | NA |
| | 90693583 | | | | |
| PACRG | chr6: 163736020- | NA | NA | NA | NA |
| | 163736524 | | | | |
| PACGRL | chr4: 20728965- | 1012.47 | −7.949803 | uc003gpz.3 | chr4: 20728965- |
| | 20729980 | | | | 20729980 |
| PACS2 | chr14: 105861010- | NA | NA | NA | NA |
| | 105864484 | | | | |
| PADI4 | NA | 439.615 | −6.715664 | uc001baj.2 | chr1: 17690251- |
| | | | | | 17690495 |
| PAFAH2 | NA | NA | NA | NA | NA |
| PAGE5 | chrX: 55250437- | NA | NA | NA | NA |
| | 55250541 | | | | |
| PAH | NA | 284.367 | −4.617248 | uc001tjq.1 | chr12: 103232104- |
| | | | | | 103232952 |
| PAK1IP1 | chr6: 10709686- | NA | NA | NA | NA |
| | 10709970 | | | | |
| PAK2 | NA | 269.032 | −4.356527 | uc003fwy.4 | chr3: 196555277- |
| | | | | | 196559518 |
| PAK6 | NA | 692.188 | −4.8243 | uc010bbl.3 | chr15: 40568296- |
| | | | | | 40569688 |
| PALM2 | chr9: 112705706- | 554.146 | −3.515224 | uc004beg.3 | chr9: 112705706- |
| | 112713756 | | | | 112713756 |
| PALMD | NA | NA | NA | NA | NA |
| PAMR1 | NA | 769.562 | −8.539456 | uc001mwf.3 | chr11: 35453376- |
| | | | | | 35453903 |
| PAN2 | chr12: 56710007- | NA | NA | NA | NA |
| | 56711392 | | | | |
| PAN3 | chr13: 28846224- | NA | NA | NA | NA |
| | 28846450 | | | | |
| PANK3 | NA | 694.513 | −7.256153 | uc003lzz.2 | chr5: 167982628- |
| | | | | | 167984575 |
| PANK4 | NA | 929.05 | −7.462958 | uc001ajm.1 | chr1: 2439975- |
| | | | | | 2440285 |
| PAPD4 | NA | 468.523 | −4.809185 | uc003kfz.2 | chr5: 78981097- |
| | | | | | 78982471 |
| PAPD5 | NA | 454.687 | −5.213643 | uc002efz.3 | chr16: 50263240- |
| | | | | | 50269219 |
| PAPSS2 | chr10: 89505728- | 887.681 | −14.360217 | uc001kew.3 | chr10: 89505728- |
| | 89507462 | | | | 89507462 |
| PAQR9 | NA | 1654.34 | −16.334787 | uc003evg.3 | chr3: 142680074- |
| | | | | | 142681044 |
| PARD3B | NA | 451.828 | −9.597776 | uc002vao.2 | chr2: 206480538- |
| | | | | | 206484886 |
| PARD6A | chr16: 67696551- | NA | NA | NA | NA |
| | 67696681 | | | | |
| PARK2 | NA | NA | NA | NA | NA |
| PARL1 | NA | NA | NA | NA | NA |
| PARP11 | NA | NA | NA | NA | NA |
| PARP12 | NA | 475.336 | −4.181136 | uc003vvl.1 | chr7: 139723549- |
| | | | | | 139724359 |
| PARP14 | NA | NA | NA | NA | NA |
| PARP3 | NA | 403.847 | −8.374723 | uc003dby.3 | chr3: 51982497- |
| | | | | | 51982883 |
| PARP4 | NA | 661.198 | −5.07902 | uc001upl.3 | chr13: 24995069- |
| | | | | | 24995256 |
| PARP8 | NA | 568.847 | −12.785208 | uc003jon.4 | chr5: 50137903- |
| | | | | | 50142356 |
| PARP9 | chr3: 122246760- | 1048.76 | −8.30011 | uc003efj.2 | chr3: 122250620- |
| | 122247210 | | | | 122254961 |
| PARPBP | NA | 202.305 | −2.644914 | uc001tjd.3 | chr12: 102542257- |
| | | | | | 102542785 |
| PARS2 | NA | 894.02 | −8.05496 | uc001cxy.3 | chr1: 55222571- |
| | | | | | 55223406 |
| PASK | chr2: 242045514- | 738.436 | −5.445168 | uc010zol.2 | chr2: 242045514- |
| | 242045980 | | | | 242045980 |
| PATE2 | chr11: 125646028- | 1269.09 | −10.344437 | uc001qcu.3 | chr11: 125646028- |
| | 125647276 | | | | 125647276 |
| PATE3 | chr11: 125660498- | NA | NA | NA | NA |
| | 125661495 | | | | |
| PAX6 | NA | 416.42 | −6.869805 | uc001mtd.4 | chr11: 31806340- |
| | | | | | 31811481 |
| PAXBP1 | NA | NA | NA | NA | NA |
| PAXIP1 | NA | NA | NA | NA | NA |
| PBX4 | NA | NA | NA | NA | NA |
| PBXIP1 | NA | NA | NA | NA | NA |
| PCCA | NA | NA | NA | NA | NA |
| PCDH10 | chr4: 134073987- | 814.106 | −11.051495 | uc003igz.3 | chr4: 134073987- |
| | 134074404 | | | | 134074404 |
| PCDH11X | NA | 670.334 | −7.876405 | uc004efj.1 | chrX: 91134306- |
| | | | | | 91134997 |
| PCDH15 | chr10: 55580860- | 773.289 | −7.566218 | uc010qhq.2 | chr10: 55562533- |
| | 55581617 | | | | 55566338 |
| PCDH17 | NA | 867.604 | −7.83288 | uc001vhq.1 | chr13: 58299429- |
| | | | | | 58303065 |
| PCDH19 | chrX: 99546642- | NA | NA | NA | NA |
| | 99551274 | | | | |
| PCDH9 | chr13: 67798639- | 767.018 | −5.125811 | uc001vin.3 | chr13: 67798639- |
| | 67799473 | | | | 67799473 |
| PCDHA1 | NA | NA | NA | NA | NA |
| PCDHAC1 | NA | 344.025 | −4.807503 | uc003lig.1 | chr5: 140308935- |
| | | | | | 140309385 |
| PCDHB10 | NA | 988.137 | −9.469104 | uc003lix.3 | chr5: 140574529- |
| | | | | | 140575213 |
| PCDHB11 | chr5: 140581742- | 1146.78 | −12.713468 | uc003liy.3 | chr5: 140581742- |
| | 140582618 | | | | 140582618 |
| PCDHB14 | chr5: 140605475- | NA | NA | NA | NA |
| | 140605860 | | | | |
| PCDHB2 | NA | 1639.48 | −17.169039 | uc003lil.3 | chr5: 140476772- |
| | | | | | 140476964 |
| PCDHB6 | chr5: 140532224- | NA | NA | NA | NA |
| | 140532868 | | | | |
| PCDHGA11 | chr5: 140803309- | NA | NA | NA | NA |
| | 140803653 | | | | |
| PCDHGA7 | chr5: 140764921- | NA | NA | NA | NA |
| | 140765187 | | | | |
| PCDHGB7 | chr5: 140799854- | 1014.9 | −10.757509 | uc003lkm.3 | chr5: 140799854- |
| | 140799996 | | | | 140799996 |
| PCF11 | NA | 536.78 | −8.415092 | uc001ozx.4 | chr11: 82895937- |
| | | | | | 82896835 |
| PCGF2 | chr17: 36890150- | 596.76 | −4.639559 | uc002hqp.1 | chr17: 36890150- |
| | 36891475 | | | | 36891475 |
| PCIF1 | NA | NA | NA | NA | NA |
| PCK2 | chr14: 24569515- | NA | NA | NA | NA |
| | 24569770 | | | | |
| PCLO | NA | 1294.92 | −12.455279 | uc003uhx.2 | chr7: 82383321- |
| | | | | | 82387890 |
| PCMT1 | chr6: 150123516- | NA | NA | NA | NA |
| | 150123667 | | | | |
| PCNX | chr14: 71478209- | NA | NA | NA | NA |
| | 71479537 | | | | |
| PCNXL3 | NA | 460.85 | −6.699406 | uc001oey.2 | chr11: 65404450- |
| | | | | | 65404910 |
| PCNXL4 | chr14: 60600940- | 1259.82 | −9.377136 | uc001xer.4 | chr14: 60600940- |
| | 60601532 | | | | 60601532 |
| PCOLCE | NA | 579.815 | −7.028651 | uc003uvo.3 | chr7: 100205727- |
| | | | | | 100205798 |
| PCP4L1 | chr1: 161254272- | NA | NA | NA | NA |
| | 161255240 | | | | |
| PCSK1 | NA | NA | NA | NA | NA |
| PCSK5 | NA | 542.177 | −7.811798 | uc004akc.2 | chr9: 78973839- |
| | | | | | 78977255 |
| PCYT1B | NA | NA | NA | NA | NA |
| PCYT2S | NA | 900.207 | −7.298237 | uc002kce.2 | chr17: 79860777- |
| | | | | | 79862708 |
| PDC | chr1: 186412698- | 1081.31 | −16.69568 | uc001grz.3 | chr1: 186412698- |
| | 186413110 | | | | 186413110 |
| PDCD2 | NA | 1312.77 | −11.507561 | uc003qxx.2 | chr6: 170887570- |
| | | | | | 170887723 |
| PDCD4 | NA | 972.356 | −9.350582 | uc001kzg.3 | chr10: 112657847- |
| | | | | | 112659764 |
| PDCD5 | NA | 225.976 | −5.017376 | uc002ntl.3 | chr19: 33076946- |
| | | | | | 33077449 |
| PDE10A | NA | 534.829 | −6.971976 | uc011egk.2 | chr6: 165740776- |
| | | | | | 165746513 |
| PDE12 | NA | 424.646 | −6.737641 | uc003diw.4 | chr3: 57545732- |
| | | | | | 57547768 |
| PDE1C | NA | NA | NA | NA | NA |
| PDE3B | NA | NA | NA | NA | NA |
| PDE4C | NA | 200.471 | −4.156455 | uc002nig.4 | chr19: 18318771- |
| | | | | | 18321738 |
| PDE4D | NA | NA | NA | NA | NA |
| PDE5A | chr4: 120415550- | 349.222 | −7.834542 | uc003idf.3 | chr4: 120415550- |
| | 120419755 | | | | 120419755 |
| PDE6B | NA | NA | NA | NA | NA |
| PDE6D | NA | 1131.11 | −9.234445 | uc002vse.1 | chr2: 232597147- |
| | | | | | 232597661 |
| PDE7A | chr8: 66626569- | NA | NA | NA | NA |
| | 66631524 | | | | |
| PDE8A | NA | 344.704 | −5.612617 | uc021stv.1 | chr15: 85681135- |
| | | | | | 85682376 |
| PDGFB | NA | NA | NA | NA | NA |
| PDGFC | NA | 1170.84 | −12.838491 | uc003iph.2 | chr4: 157682763- |
| | | | | | 157684241 |
| PDGFRA | NA | 691.075 | −8.023667 | uc003haa.3 | chr4: 55161440- |
| | | | | | 55164412 |
| PDHB | NA | NA | NA | NA | NA |
| PDHX | NA | 721.089 | −9.68529 | uc001mvt.3 | chr11: 35016720- |
| | | | | | 35017675 |
| PDIA4 | chr7: 148700154- | NA | NA | NA | NA |
| | 148700885 | | | | |
| PDIA6 | NA | 230.552 | −3.291903 | uc002rau.3 | chr2: 10923517- |
| | | | | | 10924383 |
| PDIK1L | chr1: 26449069- | NA | NA | NA | NA |
| | 26452039 | | | | |
| PDILT | NA | 832.954 | −5.092557 | NA | NA |
| PDK1 | chr2: 173460698- | 1243.19 | −9.740104 | uc002uhs.3 | chr2: 173460698- |
| | 173463862 | | | | 173463862 |
| PDLIM4 | chr5: 131607789- | NA | NA | NA | NA |
| | 131609147 | | | | |
| PDLIM7 | NA | NA | NA | NA | NA |
| PDPR | NA | NA | NA | NA | NA |
| PDS5A | NA | 1349.71 | −13.602491 | uc003guv.4 | chr4: 39824483- |
| | | | | | 39827104 |
| PDS5B | NA | 816.477 | −8.080704 | uc001uuo.3 | chr13: 33262828- |
| | | | | | 33265405 |
| PDSS1 | chr10: 27035403- | NA | NA | NA | NA |
| | 27035726 | | | | |
| PDSS2 | NA | 614.528 | −7.88281 | uc003prv.3 | chr6: 107585390- |
| | | | | | 107585623 |
| PDZD2 | NA | NA | NA | NA | NA |
| PDZD9 | NA | NA | NA | NA | NA |
| PDZK1 | NA | 498.675 | −4.754342 | uc001eon.2 | chr1: 145763624- |
| | | | | | 145764206 |
| PDZK1IP1 | chr1: 47649261- | NA | NA | NA | NA |
| | 47649642 | | | | |
| PDZRN3 | chr3: 73431652- | NA | NA | NA | NA |
| | 73432515 | | | | |
| PDZRN4 | chr12: 41967693- | NA | NA | NA | NA |
| | 41968392 | | | | |
| PEAK1 | NA | 307.931 | −5.859153 | uc021sqy.1 | chr15: 77400498- |
| | | | | | 77406497 |
| PEAR1 | chr1: 156884591- | NA | NA | NA | NA |
| | 156886226 | | | | |
| PEBP1 | chr12: 118582752- | NA | NA | NA | NA |
| | 118583387 | | | | |
| PEG10 | NA | 729.099 | −7.822272 | uc003uno.3 | chr7: 94294995- |
| | | | | | 94299006 |
| PER2 | NA | NA | NA | NA | NA |
| PET117 | NA | NA | NA | NA | NA |
| PEX1 | NA | NA | NA | NA | NA |
| PEX13 | chr2: 61275906- | NA | NA | NA | NA |
| | 61279125 | | | | |
| PEX3 | NA | 599.008 | −5.772396 | uc003qjl.3 | chr6: 143810364- |
| | | | | | 143811751 |
| PEX7 | NA | 632.878 | −6.732259 | uc003qhd.3 | chr6: 137234665- |
| | | | | | 137235072 |
| PFDN2 | chr1: 161070346- | 1221.06 | −7.257112 | uc001fxu.3 | chr1: 161070346- |
| | 161070472 | | | | 161070472 |
| PFDN4 | NA | NA | NA | NA | NA |
| PFKM | NA | 279.248 | −5.83137 | uc001rra.2 | chr12: 48539492- |
| | | | | | 48540187 |
| PFN2 | chr3: 149682691- | NA | NA | NA | NA |
| | 149683953 | | | | |
| PGAP1 | NA | 500.835 | −8.089105 | uc010fsi.3 | chr2: 197697728- |
| | | | | | 197705957 |
| PGBD1 | chr6: 28270062- | NA | NA | NA | NA |
| | 28270326 | | | | |
| PGD | NA | 601.058 | −5.720853 | uc001arc.3 | chr1: 10479807- |
| | | | | | 10480201 |
| PGK1 | chrX: 77381328- | NA | NA | NA | NA |
| | 77382324 | | | | |
| PGM3 | chr6: 83874593- | 480.632 | −0.969448 | uc003pju.2 | chr6: 83874593- |
| | 83878952 | | | | 83878952 |
| PGR | NA | NA | NA | NA | NA |
| PGRMC1 | chrX: 118377218- | 1076.48 | −10.781495 | uc004erb.3 | chrX: 118377218- |
| | 118378429 | | | | 118378429 |
| PHACTR1 | chr6: 13287311- | 486.62 | −5.192605 | uc003nai.3 | chr6: 12934125- |
| | 13288075 | | | | 12935069 |
| PHACTR4 | NA | NA | NA | NA | NA |
| PHB | NA | NA | NA | NA | NA |
| PHF1 | chr6: 33383652- | NA | NA | NA | NA |
| | 33384230 | | | | |
| PHF10 | chr6: 170107132- | NA | NA | NA | NA |
| | 170110310 | | | | |
| PHF12 | NA | 214.275 | −6.175775 | uc010wbb.1 | chr17: 27232271- |
| | | | | | 27233200 |
| PHF13 | NA | NA | NA | NA | NA |
| PHF14 | chr7: 11142126- | NA | NA | NA | NA |
| | 11147376 | | | | |
| PHF2 | chr9: 96439959- | 1177.64 | −11.466611 | uc004aub.3 | chr9: 96439959- |
| | 96441869 | | | | 96441869 |
| PHF21A | NA | NA | NA | NA | NA |
| PHF6 | chrX: 133549253- | 219.756 | −7.720945 | uc004exh.3 | chrX: 133549253- |
| | 133549321 | | | | 133549321 |
| PHF8 | chrX: 53969048- | NA | NA | NA | NA |
| | 53969796 | | | | |
| PHKG2 | NA | 236.779 | −4.357947 | uc021tgo.1 | chr16: 30771482- |
| | | | | | 30772497 |
| PHLDA1 | NA | 766.318 | −9.127107 | uc001sxu.3 | chr12: 76419227- |
| | | | | | 76423872 |
| PHLDB1 | chr11: 118527534- | NA | NA | NA | NA |
| | 118528748 | | | | |
| PHLDB3 | chr19: 43979255- | 679.976 | −6.038933 | uc002owo.3 | chr19: 44000184- |
| | 43979561 | | | | 44001254 |
| PHLPP2 | NA | 1037.13 | −7.022437 | uc002fay.1 | chr16: 71711377- |
| | | | | | 71712653 |
| PHOX2B | NA | 975.225 | −5.480359 | uc003gwf.4 | chr4: 41746099- |
| | | | | | 41747823 |
| PHTF1 | NA | 324.633 | −3.939015 | uc001edn.3 | chr1: 114246447- |
| | | | | | 114246678 |
| PHYH | chr10: 13319796- | NA | NA | NA | NA |
| | 13320300 | | | | |
| PHYHIPL | NA | 431.396 | −4.536699 | uc001jkk.4 | chr10: 61005352- |
| | | | | | 61007534 |
| PHYKPL | chr5: 177637969- | NA | NA | NA | NA |
| | 177638921 | | | | |
| PI15 | chr8: 75761489- | 581.8 | −9.912882 | uc003yal.3 | chr8: 75761489- |
| | 75767264 | | | | 75767264 |
| PI4K2B | chr4: 25278810- | NA | NA | NA | NA |
| | 25280831 | | | | |
| PI4KA | NA | 731.953 | −8.395262 | uc010gsq.2 | chr22: 21152770- |
| | | | | | 21152867 |
| PIAS2 | chr18: 44395673- | 1443.22 | −2.985424 | uc002lck.3 | chr18: 44390023- |
| | 44398339 | | | | 44392395 |
| PIAS4 | NA | 463.45 | −4.649074 | uc002lzg.3 | chr19: 4037874- |
| | | | | | 4038067 |
| PIBF1 | NA | 370.121 | −5.58409 | uc001vjb.3 | chr13: 73547862- |
| | | | | | 73548389 |
| PIEZO2 | chr18: 10670244- | NA | NA | NA | NA |
| | 10671523 | | | | |
| PIFO | NA | 1681.2 | −9.717085 | uc001eaw.2 | chr1: 111893950- |
| | | | | | 111895639 |
| PIGA | NA | 208.118 | −5.369073 | uc010neu.3 | chrX: 15337573- |
| | | | | | 15339627 |
| PIGF | chr2: 46808413- | NA | NA | NA | NA |
| | 46808730 | | | | |
| PIGG | chr4: 515555- | 506.47 | −4.991984 | uc003gam.3 | chr4: 515502- |
| | 516632 | | | | 516632 |
| PIGK | NA | 336.937 | −7.222813 | uc001dhk.3 | chr1: 77554667- |
| | | | | | 77558058 |
| PIGN | chr18: 59711458- | NA | NA | NA | NA |
| | 59713088 | | | | |
| PIGP | chr21: 38437664- | NA | NA | NA | NA |
| | 38437881 | | | | |
| PIGQ | chr16: 633036- | NA | NA | NA | NA |
| | 634136 | | | | |
| PIGU | NA | NA | NA | NA | NA |
| PIGW | NA | NA | NA | NA | NA |
| PIH1D1 | chr19: 49952349- | 1610.42 | −5.61701 | uc002pns.2 | chr19: 49949550- |
| | 49952468 | | | | 49949638 |
| PIH1D2 | chr11: 111934734- | 431.086 | −4.86249 | uc001pmp.4 | chr11: 111938523- |
| | 111934891 | | | | 111938594 |
| PIK3AP1 | NA | 543.3 | −6.519618 | uc001kmo.3 | chr10: 98353069- |
| | | | | | 98355333 |
| PIK3C2A | chr11: 17099277- | NA | NA | NA | NA |
| | 17099412 | | | | |
| PIK3CA | chr3: 178952153- | NA | NA | NA | NA |
| | 178952497 | | | | |
| PIK3CD | NA | 822.471 | −8.478028 | uc001aqb.4 | chr1: 9787105- |
| | | | | | 9789172 |
| PIK3R3 | chr1: 46505812- | 847.558 | −5.131431 | uc010olv.2 | chr1: 46505812- |
| | 46509344 | | | | 46509344 |
| PIK3R4 | NA | NA | NA | NA | NA |
| PILRA | NA | NA | NA | NA | NA |
| PIM2 | chrX: 48770459- | NA | NA | NA | NA |
| | 48771407 | | | | |
| PIN4 | NA | 255.212 | −11.282868 | uc004eam.3 | chrX: 71417377- |
| | | | | | 71418194 |
| PINK1 | NA | 652.791 | −9.061417 | uc001bdm.3 | chr1: 20977185- |
| | | | | | 20978004 |
| PIP | NA | 493.868 | −4.706335 | uc003wcf.1 | chr7: 142836736- |
| | | | | | 142836834 |
| PIP4K2A | NA | 667.946 | −7.72586 | uc010qcu.2 | chr10: 22823766- |
| | | | | | 22826129 |
| PIP5K1A | chr1: 151220342- | NA | NA | NA | NA |
| | 151222007 | | | | |
| PIP5K1P1 | chr6: 7988309- | NA | NA | NA | NA |
| | 7990577 | | | | |
| PIWIL4 | NA | NA | NA | NA | NA |
| PKD1P1 | chr16: 16443949- | NA | NA | NA | NA |
| | 16444465 | | | | |
| PKD2 | chr4: 88996847- | NA | NA | NA | NA |
| | 88998931 | | | | |
| PKDCC | chr2: 42284829- | NA | NA | NA | NA |
| | 42285668 | | | | |
| PKHD1 | NA | 501.477 | −5.478561 | uc003pah.1 | chr6: 51480145- |
| | | | | | 51483878 |
| PKIB | NA | 863.824 | −8.218676 | uc003pyz.3 | chr6: 123046341- |
| | | | | | 123047518 |
| PKNOX1 | NA | 1143.86 | −12.406823 | uc002zcq.1 | chr21: 44450212- |
| | | | | | 44453688 |
| PKP1 | NA | 901.024 | −5.987639 | uc001gwd.3 | chr1: 201299202- |
| | | | | | 201302121 |
| PKP2 | chr12: 32943680- | 715.561 | −7.275091 | uc001rlj.4 | chr12: 32943680- |
| | 32945357 | | | | 32945357 |
| PKP4 | chr2: 159537190- | 266.661 | −4.406856 | uc002tzv.3 | chr2: 159537190- |
| | 159537940 | | | | 159537940 |
| PLA1A | chr3: 119348316- | NA | NA | NA | NA |
| | 119348658 | | | | |
| PLA2G16 | NA | NA | NA | NA | NA |
| PLA2G4A | NA | 748.476 | −13.440079 | uc001gsc.3 | chr1: 186957641- |
| | | | | | 186958113 |
| PLA2G4D | NA | NA | NA | NA | NA |
| PLA2G4E | NA | 424.404 | −4.377904 | uc010udc.2 | chr15: 42273780- |
| | | | | | 42275951 |
| PLA2G5 | NA | NA | NA | NA | NA |
| PLA2G7 | chr6: 46672053- | 925.403 | −8.10754 | uc011dwd.1 | chr6: 46677401- |
| | 46672296 | | | | 46678263 |
| PLAC1 | chrX: 133699873- | NA | NA | NA | NA |
| | 133700073 | | | | |
| PLAC8 | NA | NA | NA | NA | NA |
| PLAC8L1 | chr5: 145463876- | NA | NA | NA | NA |
| | 145463994 | | | | |
| PLAU | chr10: 75676324- | NA | NA | NA | NA |
| | 75677258 | | | | |
| PLB1 | chr2: 28816921- | NA | NA | NA | NA |
| | 28818926 | | | | |
| PLCB2 | chr15: 40592870- | NA | NA | NA | NA |
| | 40593792 | | | | |
| PLCB3 | chr11: 64035029- | NA | NA | NA | NA |
| | 64036924 | | | | |
| PLCB4 | chr20: 9459637- | NA | NA | NA | NA |
| | 9461462 | | | | |
| PLCL1 | chr2: 199011687- | NA | NA | NA | NA |
| | 199014608 | | | | |
| PLD1 | NA | NA | NA | NA | NA |
| PLD5 | chr1: 242251689- | NA | NA | NA | NA |
| | 242253155 | | | | |
| PLD6 | NA | NA | NA | NA | NA |
| PLEKHA5 | NA | NA | NA | NA | NA |
| PLEKHB1 | NA | 482.49 | −5.04009 | uc001oua.3 | chr11: 73372648- |
| | | | | | 73373864 |
| PLEKHD1 | NA | 540.858 | 2.064394 | uc001xli.2 | chr14: 69967028- |
| | | | | | 69970407 |
| PLEKHG1 | chr6: 151162033- | NA | NA | NA | NA |
| | 151164799 | | | | |
| PLEKHG6 | chr12: 6437312- | NA | NA | NA | NA |
| | 6437672 | | | | |
| PLEKHH1 | chr14: 68054024- | NA | NA | NA | NA |
| | 68056255 | | | | |
| PLEKHH2 | chr2: 43937805- | NA | NA | NA | NA |
| | 43939522 | | | | |
| PLEKHJ1 | chr19: 2233155- | NA | NA | NA | NA |
| | 2233838 | | | | |
| PLEKHM2 | NA | NA | NA | NA | NA |
| PLEKHO1 | chr1: 150131719- | NA | NA | NA | NA |
| | 150131825 | | | | |
| PLEKHS1 | chr10: 115540362- | NA | NA | NA | NA |
| | 115542192 | | | | |
| PLG | chr6: 161132228- | NA | NA | NA | NA |
| | 161132904 | | | | |
| PLIN5 | NA | 1737.22 | −23.160515 | uc002mas.3 | chr19: 4522544- |
| | | | | | 4523539 |
| PLK2 | NA | NA | NA | NA | NA |
| PLK4 | NA | 325.882 | −7.774605 | uc003ifo.3 | chr4: 128819697- |
| | | | | | 128820377 |
| PLOD2 | NA | 912.902 | −5.223977 | uc003evq.1 | chr3: 145787228- |
| | | | | | 145788503 |
| PLRG1 | chr4: 155456149- | 639.613 | −5.265555 | uc003iny.3 | chr4: 155456149- |
| | 155457836 | | | | 155457836 |
| PLSCR1 | NA | 816.02 | −4.848651 | uc003ewa.2 | chr3: 146245733- |
| | | | | | 146246325 |
| PLSCR4 | NA | NA | NA | NA | NA |
| PLSCR5 | NA | NA | NA | NA | NA |
| PMAIP1 | chr18: 57569986- | NA | NA | NA | NA |
| | 57571538 | | | | |
| PMCH | NA | NA | NA | NA | NA |
| PMPCA | NA | 375.578 | −4.523741 | uc011mdy.1 | chr9: 139310985- |
| | | | | | 139311356 |
| PMPCB | chr7: 102969500- | NA | NA | NA | NA |
| | 102969958 | | | | |
| PMS2CL | NA | 462.341 | −5.703427 | uc011jxb.1 | chr7: 6777303- |
| | | | | | 6777567 |
| PMS2P5 | chr7: 74321780- | NA | NA | NA | NA |
| | 74322330 | | | | |
| PNCK | NA | 1056.52 | −8.817601 | uc010nuh.2 | chrX: 152936533- |
| | | | | | 152937576 |
| PNISR | chr6: 99847841- | NA | NA | NA | NA |
| | 99848415 | | | | |
| PNP | chr14: 20942792- | 317.124 | −5.583529 | uc001vxo.4 | chr14: 20944761- |
| | 20943322 | | | | 20946165 |
| POC1B | NA | 616.515 | −6.695257 | uc001tba.3 | chr12: 89813498- |
| | | | | | 89814929 |
| POF1B | chrX: 84532395- | 1547.27 | −11.949237 | uc004eer.2 | chrX: 84532395- |
| | 84534426 | | | | 84534426 |
| POFUT1 | NA | NA | NA | NA | NA |
| POFUT2 | NA | 418.163 | −6.061661 | uc011afq.1 | chr21: 46704937- |
| | | | | | 46705314 |
| POGLUT1 | chr3: 119211286- | NA | NA | NA | NA |
| | 119213554 | | | | |
| POGZ | NA | NA | NA | NA | NA |
| POLA2 | NA | NA | NA | NA | NA |
| POLB | chr8: 42229176- | NA | NA | NA | NA |
| | 42229331 | | | | |
| POLDIP2 | chr17: 26673659- | NA | NA | NA | NA |
| | 26675142 | | | | |
| POLG | NA | NA | NA | NA | NA |
| POLG2 | chr17: 62481711- | 872.348 | −10.775339 | uc021ubq.1 | chr17: 62473902- |
| | 62481820 | | | | 62473939 |
| POLH | NA | 1129.85 | −9.441735 | uc010jyu.2 | chr6: 43582295- |
| | | | | | 43582458 |
| POLI | NA | NA | NA | NA | NA |
| POLR1B | chr2: 113333307- | NA | NA | NA | NA |
| | 113334727 | | | | |
| POLR1E | NA | 1229.88 | −14.482919 | uc003zzy.1 | chr9: 37503200- |
| | | | | | 37503693 |
| POLR2B | chr4: 57897155- | NA | NA | NA | NA |
| | 57897334 | | | | |
| POLR2F | NA | 664.806 | −8.86666 | uc003aul.3 | chr22: 38363723- |
| | | | | | 38365319 |
| POLR2G | NA | 623.087 | −6.278528 | uc001nva.3 | chr11: 62533980- |
| | | | | | 62534187 |
| POLR2J2 | NA | NA | NA | NA | NA |
| POLR2K | chr8: 101165545- | NA | NA | NA | NA |
| | 101166230 | | | | |
| POLR3GL | chr1: 145456236- | NA | NA | NA | NA |
| | 145456636 | | | | |
| POM121 | chr7: 72419010- | 1191.66 | −10.471916 | uc010lam.1 | chr7: 72419010- |
| | 72421979 | | | | 72421979 |
| POMP | chr13: 29252240- | NA | NA | NA | NA |
| | 29253093 | | | | |
| PON3 | chr7: 94992794- | NA | NA | NA | NA |
| | 94993170 | | | | |
| POP5 | chr12: 121016848- | NA | NA | NA | NA |
| | 121017120 | | | | |
| PORCN | chrX: 48378865- | NA | NA | NA | NA |
| | 48379202 | | | | |
| POU1F1 | NA | 759.443 | −8.45257 | uc003dqq.1 | chr3: 87308783- |
| | | | | | 87309043 |
| POU3F2 | chr6: 99284082- | NA | NA | NA | NA |
| | 99286666 | | | | |
| POU4F2 | chr4: 147561961- | NA | NA | NA | NA |
| | 147563623 | | | | |
| POU6F2 | NA | NA | NA | NA | NA |
| PPA1 | chr10: 71962586- | NA | NA | NA | NA |
| | 71962910 | | | | |
| PPA2 | NA | 500.443 | −6.590136 | uc003hxl.3 | chr4: 106290234- |
| | | | | | 106290881 |
| PPAP2B | NA | 301.173 | −5.200769 | uc001cyj.2 | chr1: 56960419- |
| | | | | | 56962222 |
| PPAPDC1B | chr8: 38122258- | NA | NA | NA | NA |
| | 38122450 | | | | |
| PPARG | NA | NA | NA | NA | NA |
| PPCDC | chr15: 75341577- | NA | NA | NA | NA |
| | 75343067 | | | | |
| PPEF2 | chr4: 76781026- | NA | NA | NA | NA |
| | 76781819 | | | | |
| PPFIA2 | chr12: 81652880- | NA | NA | NA | NA |
| | 81653144 | | | | |
| PPFIBP1 | chr12: 27800818- | NA | NA | NA | NA |
| | 27800900 | | | | |
| PPIAL4E | chr1: 148644581- | 251.647 | −4.558076 | uc010paz.2 | chr1: 148644581- |
| | 148644795 | | | | 148644795 |
| PPIAP30 | chr10: 15196721- | NA | NA | NA | NA |
| | 15196797 | | | | |
| PPIB | NA | 452.163 | −5.514129 | uc010bgx.1 | chr15: 64451473- |
| | | | | | 64452261 |
| PPIC | chr5: 122359078- | 471.724 | −5.573441 | uc011cwp.1 | chr5: 122364188- |
| | 122359569 | | | | 122364831 |
| PPID | NA | 334.306 | −7.092339 | uc003iqc.3 | chr4: 159630279- |
| | | | | | 159630887 |
| PPIG | NA | 1594.8 | −6.645383 | uc002uez.3 | chr2: 170494034- |
| | | | | | 170494254 |
| PPIH | NA | 521.063 | −11.11152 | uc009vwl.2 | chr1: 43133091- |
| | | | | | 43133568 |
| PPM1A | NA | 643.807 | −7.34583 | uc001xex.4 | chr14: 60752483- |
| | | | | | 60755272 |
| PPM1B | chr2: 44457141- | 783.723 | −8.905991 | uc002rtt.3 | chr2: 44457858- |
| | 44458611 | | | | 44458611 |
| PPM1N | NA | 367.656 | −5.666301 | uc002pce.3 | chr19: 46005344- |
| | | | | | 46005764 |
| PPP1CB | chr2: 29022170- | NA | NA | NA | NA |
| | 29025806 | | | | |
| PPPICC | chr12: 111157613- | 379.001 | −2.314566 | uc001tru.3 | chr12: 111157613- |
| | 111158880 | | | | 111158880 |
| PPP1R11 | NA | 1200.73 | −7.824314 | uc003npb.3 | chr6: 30037084- |
| | | | | | 30038108 |
| PPP1R12C | NA | 417.57 | −5.702283 | uc010yfs.2 | chr19: 55602281- |
| | | | | | 55602839 |
| PPP1R14A | NA | NA | NA | NA | NA |
| PPP1R14C | NA | 375.653 | −4.184393 | uc003qnt.3 | chr6: 150569957- |
| | | | | | 150571528 |
| PPP1R15A | chr19: 49379231- | NA | NA | NA | NA |
| | 49379319 | | | | |
| PPP1R15B | NA | 751.391 | −7.435796 | uc001hav.4 | chr1: 204372492- |
| | | | | | 204375219 |
| PPP1R1A | NA | 686.872 | −7.352737 | uc001sgg.2 | chr12: 54973024- |
| | | | | | 54974170 |
| PPP1R1B | NA | 416.697 | −4.782596 | uc002hrz.3 | chr17: 37792119- |
| | | | | | 37792878 |
| PPP1R2 | NA | 651.952 | −4.406856 | uc003fup.3 | chr3: 195241221- |
| | | | | | 195243693 |
| PPP1R26 | chr9: 138379987- | 817.79 | −6.733842 | uc004cfr.1 | chr9: 138379987- |
| | 138380739 | | | | 138380739 |
| PPP1R2P3 | NA | NA | NA | NA | NA |
| PPP1R2P9 | NA | 643.785 | −7.824973 | uc004dfv.3 | chrX: 42636617- |
| | | | | | 42636721 |
| PPP1R35 | NA | NA | NA | NA | NA |
| PPP1R3D | chr20: 58511887- | NA | NA | NA | NA |
| | 58514086 | | | | |
| PPP1R7 | NA | 280.464 | −4.670793 | uc002wat.1 | chr2: 242122239- |
| | | | | | 242122439 |
| PPP2CA | chr5: 133532148- | NA | NA | NA | NA |
| | 133533462 | | | | |
| PPP2CB | NA | 348.844 | −7.741803 | uc003xik.3 | chr8: 30643126- |
| | | | | | 30643750 |
| PPP2R1B | NA | 236.984 | −5.714804 | uc001plx.1 | chr11: 111608605- |
| | | | | | 111612319 |
| PPP2R2D | NA | NA | NA | NA | NA |
| PPP2R3C | NA | 550.598 | −6.313376 | uc010amn.1 | chr14: 35567869- |
| | | | | | 35568431 |
| PPP2R4 | NA | 201.967 | −6325484 | uc004bxl.2 | chr9: 131909744- |
| | | | | | 131911225 |
| PPP2R5A | chr1: 212534113- | NA | NA | NA | NA |
| | 212535205 | | | | |
| PPP3CA | chr4: 101944587- | NA | NA | NA | NA |
| | 101947021 | | | | |
| PPP3CB | NA | NA | NA | NA | NA |
| PPP3CC | chr8: 22398316- | NA | NA | NA | NA |
| | 22398657 | | | | |
| PPP3R1 | chr2: 68405989- | NA | NA | NA | NA |
| | 68408094 | | | | |
| PPP4R2 | chr3: 73114874- | NA | NA | NA | NA |
| | 73115011 | | | | |
| PPP6R2 | chr22: 50882663- | 826.314 | −6.670711 | uc003bkz.2 | chr22: 50882677- |
| | 50883518 | | | | 50883518 |
| PPRC1 | chr10: 103909787- | 395.266 | −3.020272 | uc001kum.3 | chr10: 103909787- |
| | 103910090 | | | | 103910090 |
| PQBP1 | chrX: 48759893- | NA | NA | NA | NA |
| | 48760367 | | | | |
| PQLC1 | NA | 373.45 | −7.305904 | uc010xfm.1 | chr18: 77662420- |
| | | | | | 77664121 |
| PQLC2L | chr3: 157296123- | NA | NA | NA | NA |
| | 157296680 | | | | |
| PRADC1 | NA | NA | NA | NA | NA |
| PRAMEF1 | chr1: 12856146- | NA | NA | NA | NA |
| | 12856777 | | | | |
| PRAMEF12 | NA | NA | NA | NA | NA |
| PRAMEF20 | NA | 985.487 | −3.982038 | uc009vnv.1 | chr1: 13526874- |
| | | | | | 13526943 |
| PRAMEF5 | chr1: 13368734- | 259.67 | −5.03943 | uc001auu.1 | chr1: 13368734- |
| | 13369057 | | | | 13369057 |
| PRAMEF8 | NA | 680.226 | −7.454099 | uc001aup.3 | chr1: 12980234- |
| | | | | | 12980568 |
| PRAMEF9 | chr1: 13427860- | NA | NA | NA | NA |
| | 13428191 | | | | |
| PRCP | chr11: 82535409- | NA | NA | NA | NA |
| | 82535947 | | | | |
| PRDM1 | chr6: 106555362- | NA | NA | NA | NA |
| | 106557814 | | | | |
| PRDM10 | NA | 252.248 | −5.093698 | uc001qfj.3 | chr11: 129769601- |
| | | | | | 129772207 |
| PRDM13 | NA | 495.819 | −5.867576 | uc003pqg.1 | chr6: 100062636- |
| | | | | | 100063454 |
| PRDM15 | NA | 462.395 | −6.833814 | uc002yzo.3 | chr21: 43218385- |
| | | | | | 43221399 |
| PRDX4 | NA | NA | NA | NA | NA |
| PRELID2 | NA | 981.893 | −11.183492 | uc003lno.1 | chr5: 145138582- |
| | | | | | 145140088 |
| PREPL | NA | 516.127 | −6.958248 | uc002rug.3 | chr2: 44544748- |
| | | | | | 44548494 |
| PRIM2 | chr6: 57512703- | 282.404 | −4.578215 | uc003pdv.1 | chr6: 57190862- |
| | 57512788 | | | | 57191161 |
| PRIMA1 | chr14: 94184644- | NA | NA | NA | NA |
| | 94187789 | | | | |
| PRIMPOL | NA | NA | NA | NA | NA |
| PRKAA1 | chr5: 40759481- | NA | NA | NA | NA |
| | 40762879 | | | | |
| PRKAA2 | chr1: 57173387- | NA | NA | NA | NA |
| | 57181008 | | | | |
| PRKAB2 | chr1: 146633093- | NA | NA | NA | NA |
| | 146633248 | | | | |
| PRKACA | chr19: 14202507- | NA | NA | NA | NA |
| | 14203923 | | | | |
| PRKACB | NA | 231.107 | −4.597109 | uc001djj.3 | chr1: 84700989- |
| | | | | | 84704181 |
| PRKACG | chr9: 71627449- | NA | NA | NA | NA |
| | 71627952 | | | | |
| PRKAR1B | chr7: 588834- | NA | NA | NA | NA |
| | 590066 | | | | |
| PRKCB | NA | 638.011 | −6.791426 | uc002dme.3 | chr16: 24226138- |
| | | | | | 24231932 |
| PRKCZ | NA | 425.548 | −6.582401 | uc009vlb.3 | chr1: 2087536- |
| | | | | | 2088167 |
| PRKD3 | NA | 543.909 | −4.74564 | uc002rqf.1 | chr2: 37494711- |
| | | | | | 37496698 |
| PRKDC | chr8: 48685669- | NA | NA | NA | NA |
| | 48686733 | | | | |
| PRKG1 | NA | NA | NA | NA | NA |
| PRKRA | NA | NA | NA | NA | NA |
| PRKRIP1 | NA | NA | NA | NA | NA |
| PRKRIR | chr11: 76061004- | NA | NA | NA | NA |
| | 76061907 | | | | |
| PRKX | chrX: 3522384- | NA | NA | NA | NA |
| | 3526986 | | | | |
| PRL | chr6: 22287473- | NA | NA | NA | NA |
| | 22287630 | | | | |
| PRLHR | NA | 575.74 | −8.229101 | uc001ldp.1 | chr10: 120352916- |
| | | | | | 120353643 |
| PRMT2 | NA | NA | NA | NA | NA |
| PRMT3 | chr11: 20529960- | NA | NA | NA | NA |
| | 20530879 | | | | |
| PRNP | chr20: 4680629- | NA | NA | NA | NA |
| | 4682233 | | | | |
| PROM1 | NA | 741.01 | −5.089498 | uc003goo.2 | chr4: 15969849- |
| | | | | | 15970991 |
| PROM2 | chr2: 95954972- | NA | NA | NA | NA |
| | 95957055 | | | | |
| PROS1 | NA | 1561.58 | −9.79659 | uc010hoo.3 | chr3: 93591881- |
| | | | | | 93593088 |
| PROSER3 | NA | 1484.7 | −10.413012 | uc021usz.1 | chr19: 36259448- |
| | | | | | 36260077 |
| PRPF18 | NA | 633.934 | −7.889927 | uc001imp.3 | chr10: 13672341- |
| | | | | | 13672868 |
| PRPF38A | chr1: 52882363- | 2134.38 | −14.226575 | uc001ctw.4 | chr1: 52882433- |
| | 52883992 | | | | 52883992 |
| PRPF39 | NA | NA | NA | NA | NA |
| PRPF8 | NA | NA | NA | NA | NA |
| PRPS2 | NA | 954.167 | −5.078181 | uc004cva.3 | chrX: 12840916- |
| | | | | | 12842346 |
| PRR14 | chr16: 30667633- | NA | NA | NA | NA |
| | 30667734 | | | | |
| PRR14L | chr22: 32077334- | NA | NA | NA | NA |
| | 32081512 | | | | |
| PRR16 | NA | 1013.56 | −14.814108 | uc003ksp.3 | chr5: 120022405- |
| | | | | | 120022964 |
| PRR23A | NA | 488.808 | −5.276431 | uc011bms.2 | chr3: 138722804- |
| | | | | | 138724309 |
| PRR23B | NA | 348.47 | −5.324471 | uc003esy.1 | chr3: 138737873- |
| | | | | | 138738705 |
| PRR23C | NA | NA | NA | NA | NA |
| PRR26 | NA | 474.752 | −5.044074 | uc001ifr.3 | chr10: 708903- |
| | | | | | 711109 |
| PRR27 | NA | 470.326 | −9.784376 | uc003hfa.4 | chr4: 71028412- |
| | | | | | 71032326 |
| PRR3 | NA | NA | NA | NA | NA |
| PRR32 | NA | 717.227 | −3.771981 | uc010nra.3 | chrX: 125955519- |
| | | | | | 125955768 |
| PRR34 | NA | NA | NA | NA | NA |
| PRR9 | chr1: 153190972- | NA | NA | NA | NA |
| | 153191793 | | | | |
| PRRC1 | NA | NA | NA | NA | NA |
| PRRG1 | NA | NA | NA | NA | NA |
| PRRG3 | NA | 415.644 | −5.477706 | uc022cgt.1 | chrX: 150869506- |
| | | | | | 150870063 |
| PRRT1 | chr6: 32116140- | 310.457 | −3.945671 | uc003nzu.3 | chr6: 32116140- |
| | 32116998 | | | | 32116489 |
| PRRT2 | NA | NA | NA | NA | NA |
| PRSS22 | NA | 598.608 | −9.68033 | uc002cry.1 | chr16: 2902728- |
| | | | | | 2903093 |
| PRSS23 | NA | 1547.05 | −15.845934 | uc010rts.1 | chr11: 86519838- |
| | | | | | 86520169 |
| PRSS35 | chr6: 84234403- | NA | NA | NA | NA |
| | 84235421 | | | | |
| PRSS42 | chr3: 46871894- | NA | NA | NA | NA |
| | 46871954 | | | | |
| PRSS45 | NA | NA | NA | NA | NA |
| PRSS55 | chr8: 10411543- | NA | NA | NA | NA |
| | 10411676 | | | | |
| PRTG | NA | 253.616 | −4.894043 | uc002adg.3 | chr15: 55903739- |
| | | | | | 55912209 |
| PRUNE2 | chr9: 79226292- | NA | NA | NA | NA |
| | 79229485 | | | | |
| PRX | chr19: 40899671- | 343.447 | −4.631893 | uc002onq.3 | chr19: 40899671- |
| | 40899872 | | | | 40899872 |
| PSAP | chr10: 73576055- | 663.027 | −6.941757 | uc001jsm.3 | chr10: 73576055- |
| | 73577197 | | | | 73577197 |
| PSD3 | NA | NA | NA | NA | NA |
| PSEN2 | NA | NA | NA | NA | NA |
| PSG11 | NA | 212.223 | −4.213259 | uc002ovp.1 | chr19: 43511809- |
| | | | | | 43512262 |
| PSG3 | chr19: 43225794- | 246861 | −4.486051 | NA | NA |
| | 43226242 | | | | |
| PSG9 | NA | NA | NA | NA | NA |
| PSMA1 | NA | 388.29 | −14.081733 | uc001mlk.3 | chr11: 14526422- |
| | | | | | 14526737 |
| PSMA2 | NA | 1286.11 | −12.635253 | uc003thz.1 | chr7: 42957686- |
| | | | | | 42961452 |
| PSMA3 | chr14: 58738614- | NA | NA | NA | NA |
| | 58738727 | | | | |
| PSMA4 | chr15: 78841287- | 644.559 | −9.173064 | uc002bdu.4 | chr15: 78841287- |
| | 78841563 | | | | 78841563 |
| PSMA5 | NA | 912.351 | −9.230074 | uc001dxn.3 | chr1: 109941653- |
| | | | | | 109944634 |
| PSMA6 | NA | NA | NA | NA | NA |
| PSMB1 | NA | NA | NA | NA | NA |
| PSMB2 | chr1: 36035413- | 1165.1 | −6.010468 | uc001bzd.2 | chr1: 36035413- |
| | 36040299 | | | | 36040299 |
| PSMB3 | NA | 257.794 | −3.444616 | uc002hqr.3 | chr17: 36920421- |
| | | | | | 36920478 |
| PSMB4 | NA | NA | NA | NA | NA |
| PSMB5 | chr14: 23495060- | NA | NA | NA | NA |
| | 23495297 | | | | |
| PSMB9 | chr6: 32826348- | 515.541 | −5.459571 | uc003sga.3 | chr6: 32827310- |
| | 32826899 | | | | 32827628 |
| PSMC6 | NA | NA | NA | NA | NA |

TABLE 4

| | | Frame 1 | | | Frame 2 | | | | Frame 3 | | | | | |
|---|---|---|---|---|---|---|---|---|---|---|---|---|---|---|
| | Tumor | | | | UCSC | | | | UCSC | | | | UCSC | |
| | Associated | MIM | | | Transcript | | | | Transcript | | | | Transcript | Hg19 |
| GENE | Genes | Number | TANGO | PASTA | ID | Hg19 Location | TANGO | PASTA | ID | Hg19 Location | TANGO | PASTA | ID | Location |
| ABCB6 | Other | 605452 | NA | NA | NA | NA | NA | NA | NA | NA | 814.821 | −6.721982 | uc002vkc.2 | chr2: |
| | | | | | | | | | | | | | | 220074488- |
| | | | | | | | | | | | | | | 220074659 |
| ABL2 | Oncogene | 164690 | 239.103 | −3.707258 | uc001gmk.3 | chr1:179085983- | 222.186 | −6.350043 | uc009wxe.3 | chr1:179079716- | NA | NA | NA | NA |
| | | | | | | 179086398 | | | | 179079939 | | | | |
| AKR7A2 | Other | 603418 | NA | NA | NA | NA | 339.705 | −5.292473 | uc001bbw.3 | chr1:19629202- | NA | NA | NA | NA |
| | | | | | | | | | | 19630718 | | | | |
| AKT3 | Oncogene | 611223 | NA | NA | NA | NA | 1108.45 | −10.380657 | uc001iab.2 | chr1:243663021- | NA | NA | NA | NA |
| | | | | | | | | | | 243668550 | | | | |
| ANP32B | Other | NA | NA | NA | NA | NA | 415.007 | −8.032003 | uc004aya.3 | chr9:100777714- | NA | NA | NA | NA |
| | | | | | | | | | | 100778224 | | | | |
| BAX | Other | 600040 | 1110.13 | −6.56381 | uc002plf.1 | chr19:49464355- | 677.648 | −6.681534 | uc002plj.3 | chr19:49464894- | NA | NA | NA | NA |
| | | | | | | 49464519 | | | | 49465055 | | | | |
| BCL11A | Oncogene | 606557 | 699.926 | −2.465276 | uc002sab.3 | chr2:60678302- | NA | NA | NA | NA | 699.15 | −9.206331 | uc002sab.3 | chr2: |
| | | | | | | 60679709 | | | | | | | | 60678302- |
| | | | | | | | | | | | | | | 60679709 |
| BCL2 | Oncogene | 151430 | 664.489 | −1.553062 | uc002lit.1 | chr18:60790579- | NA | NA | NA | NA | NA | NA | NA | NA |
| | | | | | | 60795857 | | | | | | | | |
| BCL9 | Other | 602597 | NA | NA | NA | NA | 416.91 | −4.886935 | uc001epq.3 | chr1:147096761- | NA | NA | NA | NA |
| | | | | | | | | | | 147098015 | | | | |
| BCR | Other | 151410 | NA | NA | NA | NA | NA | NA | NA | NA | 802.894 | −5.02011 | uc002zww.3 | chr22: |
| | | | | | | | | | | | | | | 23657710- |
| | | | | | | | | | | | | | | 23660224 |
| BIRC5 | Other | 603352 | NA | NA | NA | NA | 208.345 | −4.748612 | uc002jvc.3 | chr17:76210933- | NA | NA | NA | NA |
| | | | | | | | | | | 76211066 | | | | |
| BUB1 | Other | 602452 | 380.956 | −6.294105 | uc002tgd.2 | chr2:111418445- | NA | NA | NA | NA | NA | NA | NA | NA |
| | | | | | | 111419154 | | | | | | | | |
| CBLB | Other | 604491 | NA | NA | NA | NA | 392.771 | −5.672775 | uc003dwa.3 | chr3:105377109- | 787.309 | −9.141429 | uc003dwa.3 | chr3: |
| | | | | | | | | | | 105377813 | | | | 105377109- |
| | | | | | | | | | | | | | | 105377813 |
| CCL2 | Other | 158105 | 422.435 | −4.526567 | uc002hhy.3 | chr17:32583847- | NA | NA | NA | NA | NA | NA | NA | NA |
| | | | | | | 32584220 | | | | | | | | |
| CCND2 | Oncogene | 123833 | NA | NA | NA | NA | 890.205 | −11.527604 | uc001qmo.3 | chr12:4409176- | 836.409 | −7.285364 | uc001qmo.3 | chr12: |
| | | | | | | | | | | 4414522 | | | | 4409176- |
| | | | | | | | | | | | | | | 4414522 |
| CCNE1 | Oncogene | 123837 | 621.915 | −6.999868 | uc002nsn.3 | chr19:30314685- | NA | NA | NA | NA | NA | NA | NA | NA |
| | | | | | | 30315215 | | | | | | | | |
| CDC5L | Oncogene | 602868 | NA | NA | NA | NA | NA | NA | NA | NA | 1213.04 | −9.123901 | uc003oxl.3 | chr6: |
| | | | | | | | | | | | | | | 44414449- |
| | | | | | | | | | | | | | | 44418161 |
| CDON | Oncogene | 608707 | NA | NA | NA | NA | 560.794 | −8.852866 | uc001qdb.4 | chr11:125826713- | NA | NA | NA | NA |
| | | | | | | | | | | 125830836 | | | | |
| CEP55 | Oncogene | 610000 | NA | NA | NA | NA | 606.546 | −8.696259 | uc009xug.3 | chr10:95287911- | NA | NA | NA | NA |
| | | | | | | | | | | 95288849 | | | | |
| CHKA | Oncogene | 118491 | NA | NA | NA | NA | NA | NA | NA | NA | 454.913 | −5.439775 | uc001onj.3 | chr11: |
| | | | | | | | | | | | | | | 67820326- |
| | | | | | | | | | | | | | | 67821454 |
| CHRM3 | Other | 118494 | 431.187 | −6.899372 | uc001hyp.3 | chr1:240072525- | NA | NA | NA | NA | NA | NA | NA | NA |
| | | | | | | 240072717 | | | | | | | | |
| CHRM5 | Other | 118496 | 632.539 | −8.422437 | uc001zhk.1 | chr15:34356518- | NA | NA | NA | NA | NA | NA | NA | NA |
| | | | | | | 34357287 | | | | | | | | |
| CRK | Oncogene | 164762 | NA | NA | NA | NA | 787.286 | −6.122336 | uc002fsl.3 | chr17:1324647- | NA | NA | NA | NA |
| | | | | | | | | | | 1326806 | | | | |
| CRKL | Oncogene | 602007 | 303.225 | −5.60498 | uc002ztf.2 | chr22:21304134- | 279.288 | −7.858606 | uc002ztf.2 | chr22:21304134- | 308.904 | −3.22987 | uc002ztf.2 | chr22: |
| | | | | | | 21308037 | | | | 21308037 | | | | 21304134- |
| | | | | | | | | | | | | | | 21308037 |
| CSF1R | Oncogene | 164770 | NA | NA | NA | NA | NA | NA | NA | NA | 688.995 | −7.060863 | uc011dcf.2 | chr5: |
| | | | | | | | | | | | | | | 149456467- |
| | | | | | | | | | | | | | | 149456695 |
| CTTN | Oncogene | 164765 | 449.083 | −6.181621 | uc001opu.3 | chr11:70282515- | 1714.88 | −10.593976 | uc001opv.4 | chr11:70281269- | 546.925 | −1.59443 | uc001opu.3 | chr11: |
| | | | | | | 70282690 | | | | 70282690 | | | | 70282515- |
| | | | | | | | | | | | | | | 70282690 |
| CYP1A1 | Other | 108330 | NA | NA | NA | NA | NA | NA | NA | NA | 1322.6 | −11.844586 | uc010bjz.1 | chr15: |
| | | | | | | | | | | | | | | 75013061- |
| | | | | | | | | | | | | | | 75013303 |
| DCUN1D1 | Oncogene | 605905 | NA | NA | NA | NA | NA | NA | NA | NA | 376.573 | −8.960392 | uc003fld.1 | chr3: |
| | | | | | | | | | | | | | | 182660559- |
| | | | | | | | | | | | | | | 182662881 |
| DDX6 | Other | 600326 | 1099.42 | −6.227878 | uc001pua.2 | chr11:118618473- | NA | NA | NA | NA | NA | NA | NA | NA |
| | | | | | | 118622806 | | | | | | | | |
| EGFL6 | Other | 300239 | 1697.5 | −12.053467 | uc004cvi.3 | chrX:13651215- | NA | NA | NA | NA | 495.428 | −9.285163 | uc004cvi.3 | chrX: |
| | | | | | | 13651694 | | | | | | | | 13651215- |
| | | | | | | | | | | | | | | 13651694 |
| EPS15 | Other | 600051 | 1288.32 | −13.627571 | uc001csp.3 | chr1:51819935- | NA | NA | NA | NA | NA | NA | NA | NA |
| | | | | | | 51822371 | | | | | | | | |
| ERBB4 | Oncogene | 600543 | 613.759 | −7.258569 | uc002veg.1 | chr2:212240442- | NA | NA | NA | NA | NA | NA | NA | NA |
| | | | | | | 212248339 | | | | | | | | |
| ETV1 | Oncogene | 600541 | 937.692 | −3.075469 | uc021zzw.1 | chr7:13946055- | 1074.2 | −5.283375 | uc021zzt.1 | chr7:13930856- | NA | NA | NA | NA |
| | | | | | | 13946137 | | | | 13935490 | | | | |
| FADD | Other | 602457 | 904.1 | −7.115195 | uc001opm.2 | chr11:70052580- | NA | NA | NA | NA | NA | NA | NA | NA |
| | | | | | | 70053508 | | | | | | | | |
| FGF20 | Oncogene | 605558 | 861.158 | −7.06114 | uc003wxc.1 | chr8:16850334- | NA | NA | NA | NA | NA | NA | NA | NA |
| | | | | | | 16850580 | | | | | | | | |
| FGFR1OP | Oncogene | 605392 | NA | NA | NA | NA | NA | NA | NA | NA | 460.806 | −9.720122 | uc011egp.1 | chr6: |
| | | | | | | | | | | | | | | 167453608- |
| | | | | | | | | | | | | | | 167453926 |
| FHL2 | Other | 602633 | 492.159 | −4.682318 | uc002tct.3 | chr2:105977283- | 1149.03 | −10.829891 | uc002tct.3 | chr2:105977283- | 1317.91 | −7.489084 | uc002tde.1 | chr2: |
| | | | | | | 105977739 | | | | 105977739 | | | | 105988004- |
| | | | | | | | | | | | | | | 105989986 |
| FUS | Oncogene | 137070 | 522.526 | −7.680538 | uc010vfi.2 | chr16:31194165- | NA | NA | NA | NA | 688.693 | −4.840821 | uc010vfi.2 | chr16: |
| | | | | | | 31194671 | | | | | | | | 31194165- |
| | | | | | | | | | | | | | | 31194671 |
| HMGA2 | Oncogene | 600698 | NA | NA | NA | NA | 257.017 | −4.072378 | uc001ssu.1 | chr12:66308695- | NA | NA | NA | NA |
| | | | | | | | | | | 66309227 | | | | |
| HMMR | Oncogene | 600936 | 1427.08 | −15.72479 | uc003lzf.3 | chr5:162918171- | 1778.43 | −23.870699 | uc003lzf.3 | chr5:162918171- | NA | NA | NA | NA |
| | | | | | | 162918953 | | | | 162918953 | | | | |
| HRAS | Oncogene | 190020 | 351.437 | −4.164841 | uc010qvw.2 | chr11:532242- | NA | NA | NA | NA | NA | NA | NA | NA |
| | | | | | | 532755 | | | | | | | | |
| IRF2 | Oncogene | 147576 | 1792.14 | −13.227448 | uc003iwf.4 | chr4:185308876- | 1609.34 | −10.44082 | uc003iwf.4 | chr4:185308876- | NA | NA | NA | NA |
| | | | | | | 185309911 | | | | 185309911 | | | | |
| IRF4 | Oncogene | 601900 | NA | NA | NA | NA | 755.367 | −6.683249 | uc010jne.2 | chr6:401816- | 496.077 | −5.244314 | uc010jne.2 | chr6:401816- |
| | | | | | | | | | | 402637 | | | | 402637 |
| KLF4 | Oncogene | 602253 | 682.472 | −5.102033 | uc004bdf.2 | chr9:110247133- | 582.265 | −4.486817 | uc004bdf.2 | chr9:110247133- | NA | NA | NA | NA |
| | | | | | | 110248031 | | | | 110248031 | | | | |
| KRAS | Oncogene | 190070 | 372.819 | −4.225845 | uc001rgp.1 | chr12:25358180- | NA | NA | NA | NA | NA | NA | NA | NA |
| | | | | | | 25362845 | | | | | | | | |
| LGALS8 | Other | 606099 | 927.675 | −10.702852 | uc001hxw.2 | chr1:236711462- | 767.442 | −7.210276 | uc001hxw.2 | chr1:236711462- | NA | NA | NA | NA |
| | | | | | | 236716279 | | | | 236716279 | | | | |
| MAF | Oncogene | 177075 | 1281.56 | −13.878332 | uc002ffm.3 | chr16:79627745- | NA | NA | NA | NA | NA | NA | NA | NA |
| | | | | | | 79628356 | | | | | | | | |
| MAPK8 | Oncogene | 601158 | NA | NA | NA | NA | 698.904 | −9.996503 | uc001jgo.3 | chr10:49642939- | NA | NA | NA | NA |
| | | | | | | | | | | 49643183 | | | | |
| MAS1 | Oncogene | 165180 | NA | NA | NA | NA | NA | NA | NA | NA | 365.428 | −5.783976 | uc003qsz.3 | chr6: |
| | | | | | | | | | | | | | | 160328966- |
| | | | | | | | | | | | | | | 160329107 |
| MCF2 | Oncogene | 311030 | NA | NA | NA | NA | 316.908 | −4.583737 | uc010nsh.2 | chrX:138663930- | NA | NA | NA | NA |
| | | | | | | | | | | 138664601 | | | | |
| MCTS1 | Oncogene | 300587 | NA | NA | NA | NA | 815.102 | −7.648517 | uc004esx.3 | chrX:119746120- | 964.019 | −9.733943 | uc004esx.3 | chrX: |
| | | | | | | | | | | 119755016 | | | | 119746120- |
| | | | | | | | | | | | | | | 119755016 |
| MERTK | Oncogene | 604705 | NA | NA | NA | NA | 1140.46 | −10.934985 | uc002thk.1 | chr2:112786442- | NA | NA | NA | NA |
| | | | | | | | | | | 112786945 | | | | |
| MLF1 | Oncogene | 601402 | 1055.4 | −14.703596 | uc003fbx.3 | chr3:158322992- | NA | NA | NA | NA | NA | NA | NA | NA |
| | | | | | | 158324249 | | | | | | | | |
| MLLT11 | Oncogene | 604684 | 964.857 | −7.940541 | uc001ewq.3 | chr1:151039974- | NA | NA | NA | NA | NA | NA | NA | NA |
| | | | | | | 151040973 | | | | | | | | |
| MLLT3 | Oncogene | 159558 | 1121.3 | −14.728143 | uc011lne.1 | chr9:20344968- | NA | NA | NA | NA | NA | NA | NA | NA |
| | | | | | | 20346440 | | | | | | | | |
| MLLT4 | Other | 159559 | 477.903 | −6.204244 | uc021zij.1 | chr6:168370616- | NA | NA | NA | NA | 751.938 | −7.985108 | uc021zij.1 | chr6: |
| | | | | | | 168372700 | | | | | | | | 168370616- |
| | | | | | | | | | | | | | | 168372700 |
| MME | Oncogene | 120520 | 558.147 | −4.926569 | uc010hvr.1 | chr3:154898249- | NA | NA | NA | NA | NA | NA | NA | NA |
| | | | | | | 154901518 | | | | | | | | |
| MTCP1 | Oncogene | 300116 | NA | NA | NA | NA | 786.117 | −8.124339 | uc004frnz.2 | chrX:154292309- | NA | NA | NA | NA |
| | | | | | | | | | | 154293674 | | | | |
| MYB | &-& Oncogene | 189990 | NA | NA | NA | NA | NA | NA | NA | NA | 651.201 | −7.516773 | uc003qfc.3 | chr6: |
| | | | | | | | | | | | | | | 135539119- |
| | | | | | | | | | | | | | | 135540311 |
| NUMA1 | Other | 164009 | 2134.83 | −12.017527 | uc001orj.2 | chr11:71713911- | NA | NA | NA | NA | 783.849 | −6.070138 | uc009ysy.2 | chr11: |
| | | | | | | 71714572 | | | | | | | | 71728045- |
| | | | | | | | | | | | | | | 71728597 |
| NUP214 | Other | 114350 | NA | NA | NA | NA | NA | NA | NA | NA | 352.165 | −6.258106 | uc010mzf.3 | chr9: |
| | | | | | | | | | | | | | | 134070759- |
| | | | | | | | | | | | | | | 134071045 |
| PARD6A | Other | 607484 | NA | NA | NA | NA | 815.795 | −5.605366 | uc002ets.3 | chr16:67696551- | NA | NA | NA | NA |
| | | | | | | | | | | 67696681 | | | | |
| PDGFB | Oncogene | 190040 | 1433.7 | −9.856361 | uc003axe.3 | chr22:39619685- | NA | NA | NA | NA | NA | NA | NA | NA |
| | | | | | | 39621318 | | | | | | | | |
| PDGFRA | Oncogene | 173490 | NA | NA | NA | NA | NA | NA | NA | NA | 6910.75 | −8.023667 | uc003haa.3 | chr4: |
| | | | | | | | | | | | | | | 55161440- |
| | | | | | | | | | | | | | | 55164412 |
| PIK3CA | Oncogene | 171834 | NA | NA | NA | NA | 269.408 | −4.710082 | uc003fjk.3 | chr3:178952153- | NA | NA | NA | NA |
| | | | | | | | | | | 178952497 | | | | |
| PMAIP1 | Other | 604959 | NA | NA | NA | NA | 893.608 | −7.57558 | uc002lic.2 | chr18:57569986- | NA | NA | NA | NA |
| | | | | | | | | | | 57571538 | | | | |
| PPP2CA | Other | 176915 | NA | NA | NA | NA | 401.543 | −6.750479 | uc003kze.3 | chr5:133532148- | NA | NA | NA | NA |
| | | | | | | | | | | 133533462 | | | | |
| PTP4A1 | Other | 601585 | NA | NA | NA | NA | NA | NA | NA | NA | 590.556 | −5.089477 | uc003pek.3 | chr6: |
| | | | | | | | | | | | | | | 64290080- |
| | | | | | | | | | | | | | | 64293489 |
| PTPN11 | Oncogene | 176876 | NA | NA | NA | NA | NA | NA | NA | NA | 228.382 | −4.798654 | uc001ttw.1 | chr12: |
| | | | | | | | | | | | | | | 112924438- |
| | | | | | | | | | | | | | | 112924727 |
| RALA | &-& Oncogene | 179550 | NA | NA | NA | NA | 1128.92 | −13.514866 | uc003thd.3 | chr7:39745845- | NA | NA | NA | NA |
| | | | | | | | | | | 39747723 | | | | |
| RBBP9 | Other | 602908 | NA | NA | NA | NA | 207.624 | −5.332478 | uc002wqy.3 | chr20:18467188- | NA | NA | NA | NA |
| | | | | | | | | | | 18470407 | | | | |
| RBM3 | Oncogene | 300027 | NA | NA | NA | NA | NA | NA | NA | NA | 232.103 | −6.420261 | uc004dkf.2 | chrX: |
| | | | | | | | | | | | | | | 48435853- |
| | | | | | | | | | | | | | | 48439553 |
| RET | Oncogene | 164761 | NA | NA | NA | NA | 969.985 | −5.931364 | uc001jak.1 | chr10:43622203- | NA | NA | NA | NA |
| | | | | | | | | | | 43622952 | | | | |
| RGS2 | Other | 600861 | 539.972 | −4.731056 | uc001gsl.3 | chr1:192780727- | NA | NA | NA | NA | NA | NA | NA | NA |
| | | | | | | 192781407 | | | | | | | | |
| RRAS2 | Other | 600098 | 446.211 | −4.701466 | uc021qec.1 | chr11:14299466- | 533.477 | −5.021064 | uc021qec.1 | chr11:14299466- | NA | NA | NA | NA |
| | | | | | | 14300882 | | | | 14300882 | | | | |
| RSU1 | Other | 179555 | NA | NA | NA | NA | 697.779 | −4.627778 | uc001iok.3 | chr10:16632617- | NA | NA | NA | NA |
| | | | | | | | | | | 16635392 | | | | |
| RUNX1 | Other | 151385 | 299.247 | −7.804626 | uc010gmw.1 | chr21:36205258- | 735.542 | −2.010638 | uc002yul.1 | chr21:36169710- | NA | NA | NA | NA |
| | | | | | | 36206416 | | | | 36170351 | | | | |
| RUNX1T1 | Oncogene | 133435 | NA | NA | NA | NA | NA | NA | NA | NA | 602.431 | −6.003529 | uc010man.2 | chr8: |
| | | | | | | | | | | | | | | 92967195- |
| | | | | | | | | | | | | | | 92972469 |
| RUNX2 | Oncogene | 600211 | NA | NA | NA | NA | NA | NA | NA | NA | 602.809 | −11.20612 | uc011dvx.2 | chr6: |
| | | | | | | | | | | | | | | 45515043- |
| | | | | | | | | | | | | | | 45518819 |
| S100A11 | Other | 603114 | NA | NA | NA | NA | 397.012 | −6.040094 | uc001ezn.3 | chr1:152004982- | NA | NA | NA | NA |
| | | | | | | | | | | 152005137 | | | | |
| SALL4 | Oncogene | 607343 | NA | NA | NA | NA | NA | NA | NA | NA | 737.856 | −8.228353 | uc002xwh.4 | chr20: |
| | | | | | | | | | | | | | | 50400583- |
| | | | | | | | | | | | | | | 50400803 |
| SEMA3E | Other | 608166 | 336.312 | −4.65747 | uc022agy.1 | chr7:82993222- | NA | NA | NA | NA | NA | NA | NA | NA |
| | | | | | | 82996901 | | | | | | | | |
| SERTAD1 | Oncogene | NA | NA | NA | NA | NA | NA | NA | NA | NA | 611.878 | −6.278893 | uc002ont.4 | chr19: |
| | | | | | | | | | | | | | | 40928409- |
| | | | | | | | | | | | | | | 40928742 |
| SERTAD2 | Oncogene | NA | 800.745 | −7.653805 | uc002sde.2 | chr2:64858755- | NA | NA | NA | NA | NA | NA | NA | NA |
| | | | | | | 64863060 | | | | | | | | |
| SETDB1 | Other | 604396 | NA | NA | NA | NA | 579.839 | −9.348083 | uc001evu.2 | chr1:150936841- | NA | NA | NA | NA |
| | | | | | | | | | | 150937220 | | | | |
| SKIL | Oncogene | 165340 | 828.136 | −11.169164 | uc003fgu.3 | chr3:170110206- | NA | NA | NA | NA | 788.168 | −10.28636 | uc003fgu.3 | chr3: |
| | | | | | | 170114637 | | | | | | | | 170110206- |
| | | | | | | | | | | | | | | 170114637 |
| SKP2 | Other | 601436 | NA | NA | NA | NA | 801.806 | −6.537313 | uc003jkc.2 | chr5:36182134- | NA | NA | NA | NA |
| | | | | | | | | | | 36184142 | | | | |
| SLC43A1 | Other | 603733 | NA | NA | NA | NA | NA | NA | NA | NA | 469.406 | −5.710395 | uc001nkk.3 | chr11: |
| | | | | | | | | | | | | | | 57252004- |
| | | | | | | | | | | | | | | 57252568 |
| SPHK1 | Oncogene | 603730 | NA | NA | NA | NA | 899.972 | −4.112876 | uc002jrf.1 | chr17:74383668- | NA | NA | NA | NA |
| | | | | | | | | | | 74383941 | | | | |
| SPI1 | Oncogene | 165170 | NA | NA | NA | NA | 639.364 | −9.461244 | uc009ylp.1 | chr11:47379732- | NA | NA | NA | NA |
| | | | | | | | | | | 47380149 | | | | |
| SRC | Oncogene | 190090 | NA | NA | NA | NA | NA | NA | NA | NA | 863.712 | −6.09356 | uc002xgx.3 | chr20: |
| | | | | | | | | | | | | | | 36031783- |
| | | | | | | | | | | | | | | 36033821 |
| SRP19 | Other | 182175 | NA | NA | NA | NA | 591.577 | −7.665719 | uc003kqc.3 | chr5:112203235- | 240.802 | −3.373497 | uc003kqb.2 | chr5: |
| | | | | | | | | | | 112204089 | | | | 112203101- |
| | | | | | | | | | | | | | | 112204089 |
| TACC3 | Other | 605303 | 1319.32 | −11.124323 | uc003gdo.3 | chr4:1746741- | NA | NA | NA | NA | 602.241 | −6.139999 | uc010ibz.3 | chr4: |
| | | | | | | 1746905 | | | | | | | | 1739718- |
| | | | | | | | | | | | | | | 1740077 |
| TAZ | Other | 300394 | 754.465 | −5.032023 | uc004fkx3 | chrX:153649344- | 614.296 | −3.166743 | uc004fkx.3 | chrX:153649344- | NA | NA | NA | NA |
| | | | | | | 153650063 | | | | 153650063 | | | | |
| TCL1B | Oncogene | 603769 | 319.376 | −3.676631 | uc001yfa.3 | chr14:96157659- | 322.924 | −3.115225 | uc001yfa.3 | chr14:96157659- | 801.36 | −7.459216 | uc001yfa.3 | chr14: |
| | | | | | | 96158965 | | | | 96158965 | | | | 96157659- |
| | | | | | | | | | | | | | | 96158965 |
| TEAD1 | Oncogene | 189967 | 596.256 | −13.238792 | uc021qdx.1 | chr11:12958770- | NA | NA | NA | NA | 439.372 | −14.086679 | uc021qdx.1 | chr11: |
| | | | | | | 12966284 | | | | | | | | 12958770- |
| | | | | | | | | | | | | | | 12966284 |
| TEC | Oncogene | 600583 | 687.467 | −7.550431 | uc003gxz.3 | chr4:48137800- | NA | NA | NA | NA | NA | NA | NA | NA |
| | | | | | | 48139432 | | | | | | | | |
| TFDP2 | Other | 602160 | NA | NA | NA | NA | 674.747 | −7.035547 | uc003euk.4 | chr3:141663270- | NA | NA | NA | NA |
| | | | | | | | | | | 141671354 | | | | |
| THRB | Oncogene | 190160 | 687.067 | −3.80782 | uc003ccx.4 | chr3:24158645- | NA | NA | NA | NA | 482.515 | −7.797255 | NA | NA |
| | | | | | | 24164374 | | | | | | | | |
| TIAM1 | Other | 600687 | NA | NA | NA | NA | 447.548 | −5.666313 | uc011adk.1 | chr21:32490736- | NA | NA | NA | NA |
| | | | | | | | | | | 32493155 | | | | |
| TPD52 | Oncogene | 604068 | 696.155 | −8.207528 | uc003ybr.1 | chr8:80947105- | NA | NA | NA | NA | NA | NA | NA | NA |
| | | | | | | 80950350 | | | | | | | | |
| TRIO | Oncogene | 601893 | 583.959 | −2.350896 | uc003jff.3 | chr5:14508532- | NA | NA | NA | NA | 1123.88 | −4.608103 | uc003jff.3 | chr5: |
| | | | | | | 14509458 | | | | | | | | 14508532- |
| | | | | | | | | | | | | | | 14509458 |
| USP4 | Oncogene | 603486 | NA | NA | NA | NA | NA | NA | NA | NA | 802.523 | −13.155383 | uc003cwp.2 | chr3: |
| | | | | | | | | | | | | | | 49314577- |
| | | | | | | | | | | | | | | 49315725 |
| WHSC1 | Oncogene | 602952 | 1638.44 | −14.749306 | uc003gdx.3 | chr4:1920396- | NA | NA | NA | NA | NA | NA | NA | NA |
| | | | | | | 1920896 | | | | | | | | |
| YES1 | Oncogene | 164880 | NA | NA | NA | NA | NA | NA | NA | NA | 481.777 | −5.685331 | uc002kky.3 | chr18: |
| | | | | | | | | | | | | | | 721592- |
| | | | | | | | | | | | | | | 724423 |
| ZNF217 | Oncogene | 602967 | NA | NA | NA | NA | 286.393 | −5.575122 | uc002xwq.4 | chr20:52183610- | NA | NA | NA | NA |
| | | | | | | | | | | 52185803 | | | | |

TABLE 5

| | | | CAE reading | | | | |
|---|---|---|---|---|---|---|---|
| GENE | OMIM # | NM # | frame | Cancer | gRNA Sequence | PAM | Exon |
| ABL2 | 164690 | NM_007314 | 1 and 2 | Glioblastoma | AAATCAGTGATGTGGTGCAG (SEQ ID NO: 1) | AGG | 12 |
| | | | | Melanoma | TGTACAGGAAATCAGTGATG (SEQ ID NO: 2) | TGG | |
| | | | | Pancreatic | | | |
| | | | | Thyroid | | | |
| BCL9 | 609004 | NM_004326 | 2 | Breast | ATGTCCATGCAGGGCATGAT (SEQ ID NO: 3) | GGG | 10 |
| | | | | Thyroid | GGGATCATGATGTTCTGTTG (SEQ ID NO: 4) | GGG | |
| BIRC5 | 603352 | NM_001168 | 2 | Thyroid | CATCGAGCAGCTGGCTGCCA (SEQ ID NO: 5) | TGG | 5 |
| | | | | | CGGCCAGAGGCCTCAATCCA (SEQ ID NO: 6) | TGG | |
| | | | | | CAGCTGGCTGCCATGGATTG (SEQ ID NO: 7) | AGG | |
| CCNE1 | 123837 | NM_001238 | 1 | Breast | AGCGGTAAGAAGCAGAGCAG (SEQ ID NO: 8) | CGG | 12 |
| | | | | Carcinoid | TAAGAAGCAGAGCAGCGGGC (SEQ ID NO: 9) | CGG | |
| | | | | Cervical | | | |
| | | | | Colorectal | | | |
| | | | | Endometrial | | | |
| | | | | Glioblastoma | | | |
| | | | | Stomach | | | |
| CEP55 | 610000 | NM_018131 | 2 | Carcinoid | TCGCGATCTGCTTGTCCATG (SEQ ID NO: 10) | TGG | 9 |
| | | | | Endometrial | ACTTTGAACAGTATTCCACA (SEQ ID NO: 11) | TGG | |
| | | | | Liver | | | |
| CSF1R | 164770 | NM_005211 | 3 | Melanoma | AACAACTATCAGTTCTGCTG (SEQ ID NO: 12) | AGG | 22 |
| | | | | Pancreatic | | | |
| FUS | 137070 | NM_004960 | 1 and 3 | Breast | TGAGCACAGACAGGATCGCA (SEQ ID NO: 13) | GGG | 15 |
| | | | | Cervical | ACAGACAGGATCGCAGGGAG (SEQ ID NO: 14) | AGG | |
| | | | | Colorectal | | | |
| | | | | Glioblastoma | | | |
| | | | | Melanoma | | | |
| | | | | Pancreatic | | | |
| IRF2 | 147576 | NM_002199 | 1 and 2 | Breast | AGGCTTAACAGCTCTTGACG (SEQ ID NO: 15) | CGG | 9 |
| | | | | Glioblastoma | GGCTTAACAGCTCTTGACGC (SEQ ID NO: 16) | GGG | |
| | | | | | GAAAACATCGGATATCACCC (SEQ ID NO: 17) | AGG | |
| MYB | 189990 | NM_005375 | 3 | Breast | ACGTGAATGCATTCTCAGCC (SEQ ID NO: 18) | CGG | 16 |
| | | | | Carcinoid | TGCATTCTCAGCCCGGACGC (SEQ ID NO: 19) | TGG | |
| | | | | Glioblastoma | TGTCTCACATGACCAGCGTC (SEQ ID NO: 20) | CGG | |
| | | | | Melanoma | | | |
| | | | | Liver | | | |
| | | | | Pancreatic | | | |
| | | | | Thyroid | | | |
| | | | | Ovarian | | | |
| NUMA1 | 164009 | NM_006185 | 1 and 3 | Breast | CTTTCAGGCAAAGCACTAAA (SEQ ID NO: 21) | GGG | 25 |
| | | | | Glioblastoma | TCTTTCAGGCAAAGCACTAA (SEQ ID NO: 22) | AGG | |
| | | | | Melanoma | CTGAAAGAGACACAGTCACA (SEQ ID NO: 23) | TGG | |
| | | | | Pancreatic | | | |
| SALL4 | 607343 | NM_020436 | 3 | Testicular | ACAAGATTGCGGTCAGCTAA (SEQ ID NO: 24) | GGG | 4 |
| | | | | Breast | CACACGCTGGACTCCTGATG (SEQ ID NO: 25) | GGG | |
| | | | | | CCGCCTGCCCAGGCACACGC (SEQ ID NO: 26) | TGG | |
| SEMA3E | 608166 | NM_012431 | 1 | Pancreatic | CAGGAGTCCAGCGTGTGCCT (SEQ ID NO: 27) | GGG | 17 |
| | | | | Liver | CTGAGCATTACCGCCTGCCC (SEQ ID NO: 28) | AGG | |
| SERTAD2 | N/A | NM_014755 | 1 | Breast | CACATCATGGAGGTGCTTGT (SEQ ID NO: 29) | TGG | 2 |
| | | | | Glioblastoma | AGAGCTGGACCACATCATGG (SEQ ID NO: 30) | AGG | |
| | | | | Pancreatic | | | |
| SPHK1 | 603730 | NM_182965 | 2 | Glioblastoma | GGTCATAAGGGCTCTTCTGG (SEQ ID NO: 31) | CGG | 8 |
| | | | | Melanoma | GCGCGGCCCAGGGGTCATAA (SEQ ID NO: 32) | GGG | |
| | | | | Liver | | | |
| TACC3 | 605303 | NM_006342 | 1 and 3 | Breast | TGGACAGCTTGTGGGAAGCG (SEQ ID NO: 33) | AGG | 6 |
| | | | | Carcinoid Lung | TCACTGCCTGGACAGCTTGT (SEQ ID NO: 34) | GGG | |
| | | | | Pancreatic | TCGCTTCCCACAAGCTGTCC (SEQ ID NO: 35) | AGG | |
| | | | | Prostate | | | |
| TIAM1 | 600687 | NM_003253 | 2 | Breast | CTCAGTGTTCAGTTTCCTGG (SEQ ID NO: 36) | AGG | 29 |
| | | | | Colorectal | TCAGTGTTCAGTTTCCTGGA (SEQ ID NO: 37) | GGG | |
| | | | | Lymphoma | CAGTGTTCAGTTTCCTGGAG (SEQ ID NO: 38) | GGG | |
| CTTN | 164765 | NM_005231 | 1, 2 and 3 | Breast | GCAGCTCCACATAGTTGGCT (SEQ ID NO: 39) | GGG | 17 |
| | | | | Colorectal | GCTCTTCCCAGCCAACTATG (SEQ ID NO: 40) | TGG | |
| | | | | Lung Ovarian | CTATGTGGAGCTGCGGCAGT (SEQ ID NO: 41) | AGG | |
| | | | | Pancreatic | TATGTGGAGCTGCGGCAGTA (SEQ ID NO: 42) | GGG | |
| | | | | Skin Thyroid | | | |
| | | | | Stomach | | | |

TABLE 6

| | Tumor | | | | UCSC | | | | UCSC | | | | UCSC | |
|---|---|---|---|---|---|---|---|---|---|---|---|---|---|---|
| | Associated | MIM | | | Transcript | | | | Transcript | | | | Transcript | Hg19 |
| GENE | Genes | Number | TANGO | PASTA | ID | Hg19 Location | TANGO | PASTA | ID | Hg19 Location | TANGO | PASTA | ID | Location |
| ABL2 | Oncogene | 164690 | 239.103 | −3.707258 | uc001gmk.3 | chr1:179085983- | 222.186 | −6.350043 | uc009wxe.3 | chr1:179079716- | NA | NA | NA | NA |
| | | | | | | 179086398 | | | | 179079939 | | | | |
| BAX | Other | 600040 | 1110.13 | −6.56381 | uc002plf.1 | chr19:49464355- | 677.648 | −6.681534 | uc0020plj.3 | chr19:49464894- | NA | NA | NA | NA |
| | | | | | | 49464519 | | | | 49465055 | | | | |
| BCL11A | Oncogene | 606557 | 699.926 | −2.465276 | uc002sab.3 | chr2:60678302- | NA | NA | NA | NA | 699.15 | −9.206331 | uc002sab.3 | chr2: |
| | | | | | | 60679709 | | | | | | | | 60678302- |
| | | | | | | | | | | | | | | 60679709 |
| BCL2 | Oncogene | 151430 | 664.489 | −1.553062 | uc002lit.1 | chr18:60790579- | NA | NA | NA | NA | NA | NA | NA | NA |
| | | | | | | 60795857 | | | | | | | | |
| BUB1 | Other | 602452 | 380.956 | −6.294105 | uc002tgd.2 | chr2:111418445- | NA | NA | NA | NA | NA | NA | NA | NA |
| | | | | | | 111419154 | | | | | | | | |
| CCL2 | Other | 158105 | 422.435 | −4.526567 | uc002hhy.3 | chr17:32583847- | NA | NA | NA | NA | NA | NA | NA | NA |
| | | | | | | 32584220 | | | | | | | | |
| CCNE1 | Oncogene | 123837 | 621.915 | −6.999868 | uc002nsn.3 | chr19:30314685- | NA | NA | NA | NA | NA | NA | NA | NA |
| | | | | | | 30315215 | | | | | | | | |
| CHRM3 | Other | 118494 | 431.187 | −6.899372 | uc001hyp.3 | chr1:240072525- | NA | NA | NA | NA | NA | NA | NA | NA |
| | | | | | | 240072717 | | | | | | | | |
| CHRM5 | Other | 118496 | 632.539 | −8.422437 | uc001zhk.1 | chr15:34356518- | NA | NA | NA | NA | NA | NA | NA | NA |
| | | | | | | 34357287 | | | | | | | | |
| CRKL | Oncogene | 602007 | 303.225 | −5.60498 | uc002ztf.2 | chr22:21304134- | 279.288 | −7.858606 | uc002ztf.2 | chr22:21304134- | 308.904 | −3.22987 | uc002ztf.2 | chr22: |
| | | | | | | 21308037 | | | | 21308037 | | | | 21304134- |
| | | | | | | | | | | | | | | 21308037 |
| CTTN | Oncogene | 164765 | 449.083 | −6.181621 | uc001opu.3 | chr11:70282515- | 1714.88 | −10.593976 | uc001opv.4 | chr11:70281269- | 546.925 | −1.59443 | uc001opu.3 | chr11: |
| | | | | | | 70282690 | | | | 70282690 | | | | 70282515- |
| | | | | | | | | | | | | | | 70282690 |
| DDX6 | Other | 600326 | 1099.42 | −6.227878 | uc001pua.2 | chr11:118618473- | NA | NA | NA | NA | NA | NA | NA | NA |
| | | | | | | 118622806 | | | | | | | | |
| EGFL6 | Other | 300239 | 1697.5 | −12.053467 | uc004cvi.3 | chrX:13651215- | NA | NA | NA | NA | 495.428 | −9.285163 | uc004cvi.3 | chrX: |
| | | | | | | 13651694 | | | | | | | | 13651215- |
| | | | | | | | | | | | | | | 13651694 |
| EPS15 | Other | 600051 | 1288.32 | −13.627571 | uc001csp.3 | chr1:51819935- | NA | NA | NA | NA | NA | NA | NA | NA |
| | | | | | | 51822371 | | | | | | | | |
| ERBB4 | Oncogene | 600543 | 613.759 | −7.258569 | uc002veg.1 | chr2:212240442- | NA | NA | NA | NA | NA | NA | NA | NA |
| | | | | | | 212248339 | | | | | | | | |
| ETV1 | Oncogene | 600541 | 937.692 | −3.075469 | uc021zzw.1 | chr7:13946055- | 1074.2 | −5.283375 | uc021zzt.1 | chr7:13930856- | NA | NA | NA | NA |
| | | | | | | 13946137 | | | | 13935490 | | | | |
| FADD | Other | 602457 | 904.1 | −7.115195 | uc001opm.2 | chr11:70052580- | NA | NA | NA | NA | NA | NA | NA | NA |
| | | | | | | 70053508 | | | | | | | | |
| FGF20 | Oncogene | 605558 | 861.158 | −7.06114 | uc003wxc.1 | chr8:16850334- | NA | NA | NA | NA | NA | NA | NA | NA |
| | | | | | | 16850580 | | | | | | | | |
| FHL2 | Other | 602633 | 492.159 | −4.682318 | uc002tct.3 | chr2:105977283- | 1149.03 | −10.829891 | uc002tct.3 | chr2:105977283- | 1317.91 | −7.489084 | uc002tde.1 | chr2: |
| | | | | | | 105977739 | | | | 105977739 | | | | 105988004- |
| | | | | | | | | | | | | | | 105989986 |
| FUS | Oncogene | 137070 | 522.526 | −7.680538 | uc010vfi.2 | chr16:31194165- | NA | NA | NA | NA | 698.693 | −4.840821 | uc010 [symbol] | chr: |
| | | | | | | 31194671 | | | | | | | | 1631194165- |
| | | | | | | | | | | | | | | 31194671 |
| HMMR | Oncogene | 600936 | 1427.08 | −15.72479 | uc003lzf.3 | chr5:162918171- | 1778.43 | −23.870699 | uc003lzf.3 | chr5:162918171- | NA | NA | NA | NA |
| | | | | | | 162918953 | | | | 162918953 | | | | |
| HRAS | Oncogene | 190020 | 351.437 | −4.164841 | uc010qvw.2 | chr11:532242- | NA | NA | NA | NA | NA | NA | NA | NA |
| | | | | | | 532755 | | | | | | | | |
| IRF2 | Oncogene | 147576 | 1792.14 | −13.227448 | uc003iwf.4 | chr4:185308876- | 1609.34 | −10.44082 | uc003iwf.4 | chr4:185308876- | NA | NA | NA | NA |
| | | | | | | 185309911 | | | | 185309911 | | | | |
| KLF4 | Oncogene | 602253 | 682.472 | −5.102033 | uc004bdf.2 | chr9:110247133- | 582.265 | −4.486817 | uc004bdf.2 | chr9:110247133- | NA | NA | NA | NA |
| | | | | | | 110248031 | | | | 110248031 | | | | |
| KRAS | Oncogene | 190070 | 372.819 | −4.225845 | uc001rgp.1 | chr12:25358180- | NA | NA | NA | NA | NA | NA | NA | NA |
| | | | | | | 25362845 | | | | | | | | |
| LGALS8 | Other | 606099 | 927.675 | −10.702852 | uc001hxw.2 | chr1:236711462- | 767.442 | −7.210276 | uc001hxw.2 | chr1:236711462- | NA | NA | NA | NA |
| | | | | | | 236716279 | | | | 236716279 | | | | |
| MAF | Oncogene | 177075 | 1281.56 | −13.878332 | uc002ffm.3 | chr16:79627745- | NA | NA | NA | NA | NA | NA | NA | NA |
| | | | | | | 79628356 | | | | | | | | |
| MLF1 | Oncogene | 601402 | 1055.4 | −14.703596 | uc003fbx.3 | chr3:158322992- | NA | NA | NA | NA | NA | NA | NA | NA |
| | | | | | | 158324249 | | | | | | | | |
| MLLT11 | Oncogene | 604684 | 964.857 | −7.940541 | uc001ewq.3 | chr1:151039974- | NA | NA | NA | NA | NA | NA | NA | NA |
| | | | | | | 151040973 | | | | | | | | |
| MLLT3 | Oncogene | 159558 | 1121.3 | −14.728143 | uc011lne.1 | chr9:20344968- | NA | NA | NA | NA | NA | NA | NA | NA |
| | | | | | | 20346440 | | | | | | | | |
| MLLT4 | Other | 159559 | 447.903 | −6.204244 | uc021zij.1 | chr6:168370616- | NA | NA | NA | NA | 751.938 | −7.985108 | uc021zij.1 | chr6: |
| | | | | | | 168372700 | | | | | | | | 168370616- |
| | | | | | | | | | | | | | | 168372700 |
| MME | Oncogene | 120520 | 558.147 | −4.926569 | uc010hvr.1 | chr3:154898249- | NA | NA | NA | NA | NA | NA | NA | NA |
| | | | | | | 154901518 | | | | | | | | |
| NUMA1 | Other | 164009 | 2134.83 | −12.017527 | uc001orj.2 | chr11:71713911- | NA | NA | NA | NA | 783.849 | −6.070138 | uc009ysy.2 | chr11: |
| | | | | | | 71714572 | | | | | | | | 71728045- |
| | | | | | | | | | | | | | | 71728597 |
| PDGFB | Oncogene | 190040 | 1433.7 | −9.856361 | uc003axe.3 | chr22:39619685- | NA | NA | NA | NA | NA | NA | NA | NA |
| | | | | | | 39621318 | | | | | | | | |
| RGS2 | Other | 600861 | 539.972 | −4.731056 | uc001gsl.3 | chr1:192780727- | NA | NA | NA | NA | NA | NA | NA | NA |
| | | | | | | 192781407 | | | | | | | | |
| RRAS2 | Other | 600098 | 446.211 | −4.701466 | uc021qec.1 | chr11:14299466- | 533.477 | −5.021064 | uc021qec.1 | chr11:14299466- | NA | NA | NA | NA |
| | | | | | | 14300882 | | | | 14300882 | | | | |
| RUNX1 | Other | 151385 | 299.247 | −7.804626 | uc010gmw.1 | chr21:36205258- | 735.542 | −2.010638 | uc002yul.1 | chr21:36169710- | NA | NA | NA | NA |
| | | | | | | 36206416 | | | | 36170351 | | | | |
| SEMA3E | Other | 608166 | 336.312 | −4.65747 | uc022agy.1 | chr7:82993222- | NA | NA | NA | NA | NA | NA | NA | NA |
| | | | | | | 82996901 | | | | | | | | |
| SERTAD2 | Oncogene | NA | 800.745 | −7.653805 | uc002sde.2 | chr2:64858755- | NA | NA | NA | NA | NA | NA | NA | NA |
| | | | | | | 64863060 | | | | | | | | |
| SKIL | Oncogene | 165340 | 828.136 | −11.169164 | uc003fgu.3 | chr3:170110206- | NA | NA | NA | NA | 788.168 | −10.28636 | uc003fgu.3 | chr3: |
| | | | | | | 170114637 | | | | | | | | 170110206- |
| | | | | | | | | | | | | | | 170114637 |
| TACC3 | Other | 605303 | 1319.32 | −11.124323 | uc003gdo.3 | chr4:1746741- | NA | NA | NA | NA | 602.241 | −6.139999 | uc010ibz.3 | chr4: |
| | | | | | | 1746905 | | | | | | | | 1739718- |
| | | | | | | | | | | | | | | 1740077 |
| TAZ | Other | 300394 | 754.465 | −5.032023 | uc004fkx.3 | chrX:153649344- | 614.296 | −3.166743 | uc004fkx.3 | chrX:153649344- | NA | NA | NA | NA |
| | | | | | | 153650063 | | | | 153650063 | | | | |
| TCL1B | Oncogene | 603769 | 319.376 | −3.676631 | uc001yfa.3 | chr14:96157659- | 322.924 | −3.115225 | uc001yfa.3 | chr14:96157659- | 801.36 | 7.459216 | uc001yfa.3 | chr14: |
| | | | | | | 96158965 | | | | 96158965 | | | | 96157659- |
| | | | | | | | | | | | | | | 96158965 |
| TEAD1 | Oncogene | 189967 | 596.256 | −13.238792 | uc021qdx.1 | chr11:12958770- | NA | NA | NA | NA | 439.372 | −14.086679 | uc021qdx.1 | chr11: |
| | | | | | | 12966284 | | | | | | | | 12958770- |
| | | | | | | | | | | | | | | 12966284 |
| TEC | Oncogene | 600583 | 687.467 | −7.550431 | uc003gxz.3 | chr4:48137800- | NA | NA | NA | NA | NA | NA | NA | NA |
| | | | | | | 48139432 | | | | | | | | |
| THRB | Oncogene | 190160 | 687.067 | −3.80782 | uc003ccx.4 | chr3:24158645- | NA | NA | NA | NA | 482.515 | −7.797255 | NA | NA |
| | | | | | | 24164374 | | | | | | | | |
| TPD52 | Oncogene | 604068 | 696.155 | −8.207528 | uc003ybr.1 | chr8:80947105- | NA | NA | NA | NA | NA | NA | NA | NA |
| | | | | | | 80950350 | | | | | | | | |
| TRIO | Oncogene | 601893 | 583.959 | −2.350896 | uc003jff.3 | chr5:14508532- | NA | NA | NA | NA | 1123.88 | −4.608103 | uc003jff.3 | chr5: |
| | | | | | | 14509458 | | | | | | | | 14508532- |
| | | | | | | | | | | | | | | 14509458 |
| WHSC1 | Oncogene | 602952 | 1638.44 | −14.749306 | uc003gdx.3 | chr4:1920396- | NA | NA | NA | NA | NA | NA | NA | NA |
| | | | | | | 1920896 | | | | | | | | |

[symbol]

indicates data missing or illegible when filed

1. A method of treating a mammalian subject to inhibit growth of neoplastic cells comprising:
administering to the subject a composition that comprises an agent selected from the group consisting of:
a. an agent that comprises an amyloidogenic peptide;
b. an agent that comprises a nucleotide sequence that encodes the amyloidogenic peptide; and
c. an agent that induces expression of an endogenous nucleotide sequence that encodes the amyloidogenic peptide;
wherein the amyloidogenic peptide is either:
(i) a Cryptic Amyloidogenic Element (“CAE”) encoded by nucleic acid in a 3′-untranslated region (“3′-UTR”) of a gene in the neoplastic cell; or
(ii) comprises an amino acid sequence at least 70% identical to the CAE and retains the property of amyloidogenic aggregation.

2-3. (canceled)

4. The method according to claim 1, wherein the agent is administered in an amount effective to kill malignant neoplastic cells in the subject.

5. The method according to claim 1, wherein the mammalian subject is human.

6. (canceled)

7. The method according to claim 1, wherein the neoplastic cells are from a cancer of neurogenic origin or a glia-derived tumor.

8. The method according to claim 7, wherein the cancer is selected from the group consisting of neuroblastomas, central neurocytomas, and retinoblastomas.

9. The method according to claim 8, wherein the amyloidogenic peptide is encoded by the 3′-UTR of an NEFH or NEFL gene.

10. (canceled)

11. The method according to claim 1, further comprising:
screening the neoplastic cells to identify one or more genes that are highly expressed therein; and
selecting, as the amyloidogenic peptide, a peptide that is encoded by a CAE that is located in the 3′-UTR of one of the highly expressed genes, wherein the selecting of the amyloidogenic peptide comprises analyzing the 3′-UTR of the genes identified as highly expressed and selecting a gene having a 3′-UTR containing an open reading frame that encodes a peptide with amyloidogenic properties.

12-15. (canceled)

16. The method according to claim 1, wherein the CAE is present in the 3′-UTR of a gene set forth in Table 1.

17. The method according to claim 1, wherein the CAE is in the 3′-UTR of gene is selected from the group consisting of APOA2, ATXN2, B2M, FGB, FUS, IAPP, LYZ, NOTCH3, PRNP, RHO, SAA1, SNCA, NEFH, NEFL, BSCL2, MAPT, and TTR.

18-20. (canceled)

21. The method according to claim 1, wherein the agent comprises a nucleotide sequence that encodes the amyloidogenic peptide.

22. (canceled)

23. The method according to claim 21, wherein the agent further comprises a promoter operatively linked to the nucleotide sequence that encodes the amyloidogenic peptide, wherein the promoter is a tissue-selective promoter that promotes expression of genes in the neoplastic cells.

24. (canceled)

25. The method according to claim 23, wherein the agent comprises a vector that comprises the nucleotide sequence that encodes the amyloidogenic peptide.

26. (canceled)

27. The method according to claim 1, wherein the amyloidogenic peptide comprises the CAE, and the agent causes expression of an endogenous nucleotide sequence that encodes the CAE,
wherein the agent causes expression by causing translation of mRNA beyond the regular stop codon of the gene; or
wherein the agent comprises an aminoglycoside antibiotic that induces translational read-through; or
wherein the agent induces expression of the amyloidogenic peptide by modifying chromosomal DNA of the neoplastic cells to create a modified gene that includes the CAE in-frame with the start codon, uninterrupted by a stop codon; or
wherein the agent comprises a CRISPR Cas9 protein and one or more guide RNA molecules to introduce a site-specific modification of the chromosomal DNA to create a modified gene that includes the CAE in-frame with the start codon, uninterrupted by a stop codon; or
wherein the agent comprises a zinc finger nuclease or a transcription activator-like effector nuclease (TALEN) to introduce a site-specific modification of the chromosomal DNA to create a modified gene that includes the CAE in-frame with the start codon, uninterrupted by a stop codon.

28-35. (canceled)

36. The method according to claim 1, wherein the tumor is in the eye or brain and the agent is administered intracranially or intravitrially.

37. An expression vector comprising a polynucleotide comprising a nucleotide sequence that encodes an amyloidogenic peptide fused in-frame with an expression control sequence to promote expression of the amyloidogenic peptide in mammalian cells,
wherein the amyloidogenic peptide is either:
(i) a Cryptic Amyloidogenic Element (“CAE”) encoded by nucleic acid in a 3′-untranslated region (“3′-UTR”) of a gene set forth in Table 1 in the neoplastic cell; or
(ii) comprises an amino acid sequence at least 80% identical to the CAE and retains the property of amyloidogenic aggregation.

38. (canceled)

39. The polynucleotide according to claim 37, wherein the CAE is in the 3′-UTR of gene is selected from the group consisting of APOA2, ATXN2, B2M, FGB, FUS, IAPP, LYZ, NOTCH3, PRNP, RHO, SAA1, SNCA, NEFH, NEFL, BSCL2, MAPT, and TTR.

40. (canceled)

41. An expression vector comprising the polynucleotide according to claim 37.

42-48. (canceled)

49. A polynucleotide comprising:
(a) a nucleotide sequence of a crRNA sequence, said crRNA sequence including:
(i) a nucleotide targeting sequence of 17-20 nucleotides that hybridizes to a 3′ end (“target site”) of a gene set forth in Table 1, and
(ii) a nucleotide sequence that hybridizes to a tracrRNA recognized by a Cas9 protein,
wherein (i) is disposed 5′ relative to (ii) and
wherein the targeting sequence targets a site inclusive or upstream of, and within 100 nucleotides of, the wild type stop codon of the gene, said site being immediately upstream of a protospacer adjacent motif (PAM) sequence for said Cas9 protein; or
(b) a nucleotide sequence encoding (a).

50-54. (canceled)

55. An expression vector comprising the polynucleotide according to claim 49, wherein the nucleotide sequence that encodes the polynucleotide is operably linked to an expression control sequence to promote expression of the polynucleotide as RNA in a human cell.

56-70. (canceled)

71. A composition comprising a polynucleotide according to claim 49, or a nanoparticle comprising the polynucleotide, and a pharmaceutically acceptable carrier.

72. (canceled)
